(19) United States
(12) Patent Application Publication
(10) Pub. No.: US 2026/0069715 A1
(43) Pub. Date: Mar. 12, 2026

(54) COMPOSITIONS AND METHODS FOR THE TREATMENT OF HUNTINGTONS DISEASE BY EDITING THE MUTANT HUNTINGTIN GENE

(71) Applicant: LifeEDIT Therapeutics, Inc., Durham, NC (US)

(72) Inventors: Philip Borden, Durham, NC (US); Logan Brown, Raleigh, NC (US); Nancy Cheng, Chapel Hill, NC (US); Alexandra Briner Crawley, Cary, NC (US); Tedd D. Elich, Durham, NC (US); Clayton Morrison, Apex, NC (US); Ariel Vitenzon, Durham, NC (US); Kathryn Woodburn, Hillsborough, NC (US)

(73) Assignee: LifeEDIT Therapeutics, Inc., Durham, NC (US)

(21) Appl. No.: 18/852,277

(22) PCT Filed: Apr. 12, 2024
(86) PCT No.: PCT/IB2024/053622

§ 371 Date: Sep. 27, 2024

Related U.S. Application Data
(60) Provisional application No. 63/495,725, filed on Apr. 12, 2023.
(60) Provisional application No. 63/497,904, filed on Apr. 24, 2023.
(60) Provisional application No. 63/518,231, filed on Aug. 8, 2023.
(60) Provisional application No. 63/593,881, filed on Oct. 27, 2023.
(60) Provisional application No. 63/555,290, filed on Feb. 19, 2024.

(51) Int. Cl. A61K 48/00; C12N 9/22; C12N 15/11; C12N 15/86; G01N 33/68

(52) U.S. Cl. CPC A61K 48/0058; C12N 9/226; C12N 15/11; C12N 15/86; G01N 33/6896; C12N 2310/20; C12N 2750/14143; C12N 2800/22; G01N 2800/2835

(57) ABSTRACT

Compositions include CRISPR RNAs, guide RNAs, and nucleic acid molecules encoding the same. Vectors and host cells comprising the nucleic acid molecules are also provided. Further provided are RNA-guided nuclease (RGN) systems for cleaving a mutHTT allele, wherein the RGN system comprises an RNA-guided nuclease and a guide RNA. The compositions find use in cleaving or modifying a mutHTT allele, and/or modifying the expression of a mutHTT allele. The compositions are additionally useful for treating Huntington's disease (HD), particularly in an allele-specific manner.

### CROSS-REFERENCE TO RELATED APPLICATIONS

This application claims priority to U.S. Provisional Application No. 63/495,725, filed Apr. 12, 2023, U.S. Provisional Application No. 63/497,904, filed Apr. 24, 2023, U.S. Provisional Application No. 63/518,231, filed Aug. 8, 2023, U.S. Provisional Application No. 63/593,881, filed Oct. 27, 2023, and U.S. Provisional Application No. 63/555,290, filed Feb. 19, 2024, each of which is incorporated by reference herein in its entirety.

### REFERENCE TO A SEQUENCE LISTING SUBMITTED ELECTRONICALLY AS AN XML FILE

The instant application contains a Sequence Listing which has been submitted in xml format via USPTO Patent Center and is hereby incorporated by reference in its entirety. Said xml copy, created on Apr. 10, 2024, is named L103438_1300WO_0257_6_SL, and is 251,299 bytes in size.

### FIELD OF THE INVENTION

The present invention relates to the field of molecular biology and gene editing.

### BACKGROUND OF THE INVENTION

Huntington's disease (HD) is an inherited neurodegenerative disorder caused by a cytosine-adenine-guanine (CAG) trinucleotide expansion in the huntingtin (HTT) gene (Huntington's Disease Collaborative Research Group, 1993, Cell 72:971-983). The resulting polyglutamine (polyQ) containing mutant HTT disrupts wild-type HTT (wtHTT) functions which results in neural stress and malfunction (Kaemmerer et al, 2019, Degenerative Neurological and Neuromuscular Disease 9:3-17). HD patients develop striatum atrophy along with cognitive impairment followed by progressive psychiatric and motor deficits (Ross et al., 2014, Nat. Rev. Neurol. 10:204-216). Murine models expressing either full length or exon 1 of human HTT containing expanded CAG repeats recapitulate HD pathophysiology (Southwell et al., 2016, Human Molecular Genetics 25(17):3654-3675; Slow et al., 2003, Human Molecular Genetics 12(13):1555-1567; Raamsdonk et al, 2007, Neurobiology of Disease 26:189-200; Southwell et al., 2017, Human Molecular Genetics 26(6):1115-1132). Reducing mutant HTT levels in HD animal models resulting in the amelioration of motor and neuropathological abnormalities, supports HTT-lowering as a therapeutic approach (Miniarikova et al., 2016, Mol Ther Nucleic Acids 5(3):e297; Caron et al., 2020, Nucleic Acids Research 48(1):36-54; Spronck et al., 2019, Mol Ther Methods Clin Dev 13:334-343; Stanek et al., 2014, Human Gene Therapy 25(5):461-474).

While RNA interference is being developed to reduce mutant huntingtin protein levels, RNAi treatment does not completely eliminate mutant huntingtin expression. Targeted genome editing provides an opportunity to introduce changes at the genomic level. Targeted genome editing or modification is rapidly becoming an important tool for basic and applied research, as it allows modification of genomes such as cutting nucleic acids, deleting nucleic acids, inserting nucleic acids, substituting nucleotides in nucleic acids, and regulating gene expression at specific locations in a genome, along with many other possible modifications. Initial efforts in genome editing involved designing nucleases, proteins that are able to edit nucleic acids, to recognize and bind specifically to a target nucleic acid sequence to be edited. However, engineering nucleases takes considerable time and experimentation to obtain ones effective for editing of a particular sequence. Genome editing systems that use RNA-guided nucleases, such as the Clustered Regularly Interspaced Short Palindromic Repeats (CRISPR)-associated (Cas) proteins of the CRISPR-Cas bacterial system, function by complexing a nuclease with a guide RNA. The hybridization of the guide RNA to a particular target sequence allows editing at a specific location in a genome. Thus, genome editing systems that use RNA-guided nucleases can be less costly and more efficient for editing of genome sequences, as nucleic acids typically can be easier to design and re-design as compared to a nuclease.

Thus, patients afflicted with diseases such as Huntington's disease that are associated with specific defects in the genome, would benefit from the development of RNA-guided nuclease systems that are able to edit the genomic defect for therapeutic purposes.

### BRIEF SUMMARY OF THE INVENTION

Compositions and methods for cleaving a mutant huntingtin (mutHTT) allele are provided. Compositions include CRISPR RNAs, guide RNAs, and nucleic acid molecules encoding the same. Vectors and host cells comprising the nucleic acid molecules are also provided. Further provided are RNA-guided nuclease (RGN) systems for cleaving a mutHTT allele, wherein the RGN system comprises an RNA-guided nuclease and a guide RNA. The compositions find use in cleaving or modifying a mutHTT allele, and/or modifying the expression of a mutHTT allele. The compositions are additionally useful for treating Huntington's disease (HD), particularly in an allele-specific manner.

Methods for cleaving a mutHTT allele in a cell comprise introducing an RGN or a nucleic acid molecule encoding the RGN, and a guide RNA or a nucleic acid molecule encoding the guide RNA, wherein the mutHTT allele comprises a single nucleotide polymorphism (SNP) allele in exon 50, wherein said the SNP allele generates a protospacer adjacent motif (PAM), and wherein the RGN is capable of recognizing the PAM and cleaving the mutHTT allele.

Methods for ameliorating or delaying the onset of one or more symptoms of HD in a subject in need thereof comprise administering to the subject an RGN system, wherein the mutHTT allele of said subject comprises a SNP allele in exon 50 that generates a protospacer adjacent motif (PAM) recognized by the RGN. The RGN then cleaves and edits the mutHTT allele, and levels of a mutHTT protein encoded by the mutHTT allele are reduced compared to a control subject or wild type HTT protein.

### BRIEF DESCRIPTION OF THE FIGURES

FIG. 1 shows the percent insertions and/or deletions (INDELs) in patient fibroblasts nucleofected with the APG07433.1 nuclease and SGN002908 or SGN002911 guide RNA, or with the APG05586 nuclease and SGN004282 guide RNA.

FIGS. 2A and 2B show the percent INDELs and percent of edited reads, respectively, in patient fibroblasts nucleofected with the APG05586 nuclease and SGN004282, SGN008949, or SGN007707 guide RNA. (FIG. 2B) No edited reads were detected for the C-allele in any of the tested patient fibroblasts using APG05586 nuclease and SGN004282 guide RNA.

FIGS. 3A-3B provide an immunofluorescent analysis of neuronal marker genes in induced pluripotent stem cell (iPSC) derived forebrain neurons. FIG. 3A (left panel) shows iPSC-derived forebrain neurons cells express the neuron markers Tuj1 (β Tubulin III, Green) and gamma-aminobutyric acid (GABA) (Red), magnification 10×; (middle panel) shows cells also express the neuron markers Tuj1 (Green) and microtubule-associated protein 2 (MAP2) (Red) which is a maturated neuron marker, magnification 10×; (right panel) shows iPSC-derived cells express Tuj1 (Green) and forebrain neuron specific marker, FoxG1 (Red), magnification 20×. Nuclei are labeled with DAPI (Blue). FIG. 3B shows FACS analysis on iPSC-derived forebrain neurons. The negative control is shown in the far left panel. The mid left to far right panels display the results of staining with the following antibody combinations: Ki67 and neurofilament heavy chain (NEFH) (mid left); GABA and MAP2 (mid right); and GFAP and Tuj1 (far right).

FIG. 4 provides an immunofluorescent analysis of neuronal marker genes cAMP regulated phosphoprotein of apparent molecular weight 32 kDa (DARPP-32), GABA, MAP2, or Ctip2 in iPSC derived medium spiny neurons.

FIG. 5 provides a graph showing the percent INDELs in induced pluripotent stem cells (iPSCs), neural progenitor cells (NPCs), forebrain neuron progenitors (FBPs), or forebrain neurons (FBNs) using the indicated guide RNA (and appropriate nuclease) as described in FIG. 1.

FIG. 6 depicts AAV5-mediated APG07433.1 intrastriatal delivery resulting in substantial levels of AAV5 vector DNA in the clinically relevant brain regions, striatum and cortex. Dose response was observed at both 4 weeks and 3 months post-administration. Each point represents individual mice with mean±SE shown. Naïve and vehicle treated animals are not depicted as they are below lower limit of quantitation (LLOQ). vg=viral genomes.

FIG. 7 shows AAV5-mediated APG07433.1 intrastriatal delivery resulting in strong APG07433.1 transgene expression 4 weeks post-administration. dPCR analysis from right striatum. Each point represents individual mice with mean±SE shown. [n=3-6 per group].

FIG. 8 demonstrates dose-dependent reduction in mutant HTT protein in the striatum at 4 weeks and 3 months post intrastriatal AAV5-JeT-APG07433.1-SGN002908 administration. Each point represents individual mice with mean±SE shown. [n=2-6 per group for 4-weeks; n=2-10 per group for the 3-month timepoints]. **P<0.01, ****P<0.0001.

FIG. 9 shows mutant HTT mRNA dose-dependent reduction when evaluated 3 months post-intrastriatal administration of AAV5-JeT-APG07433.1-SGN002908. Each point represents individual mice with mean±SE shown. [n=2-10 per group]. *P<0.05; **P<0.01 compared to concurrent naïve animals.

FIG. 10 provides results of an INDEL next generation sequencing (NGS) analysis that demonstrates editing in striata of animals treated with 6.4E10 vg and 3.6E11 vg AAV5-JeT-APG07433.1-SGN002908 4 weeks and 3 months post administration. Each point represents individual mice with mean±SE shown. [n=2-4 per group for 4-weeks; n=2-10 per group for 3-months].

FIG. 11 shows intrastriatal injection of 1.72E11 vg AAV5-hU6-SGN004282-JeT-APG05586 resulted in robust AAV5 vector levels within the striatum resulting in APG05586 nuclease editing and 30 percent reduction in mutHTT protein. Each point represents individual animals with mean±SE. Naïve and vehicle cohorts were below the limit of quantitation for vector biodistribution. *P<0.05, unpaired t-test versus concurrent naïve control cohort.

FIG. 12 shows vector genomic biodistribution within the striatum and cortex following intrastriatal delivery with subsequent APG05586 nuclease expression. Each point represents individual animals with mean±SE. Naïve cohorts were below the limit of quantitation for vector biodistribution.

FIG. 13 shows AAV5-hU6-SGN004282-JeT-APG05586 and AAV5-hU6-SGN004282-hSyn-APG05586 intrastriatal administration caused mutHTT protein reduction and confirmed editing. Each point represents individual animals with mean±SE. **P<0.01 compared to concurrent naïve animal using unpaired Students t-test.

FIG. 14 shows intrastriatal delivery of 2.84E11 vg AAV5-hU6-SGN004282-hSyn-APG05586 resulted in extensive vector genomic disposition within the striatum resulting in APG05586 nuclease expression. Each point represents individual animals with mean±SE. Naïve cohorts were below the limit of quantitation for vector biodistribution.

FIG. 15 shows AAV5-hU6-SGN004282-hSyn-APG05586 intrastriatal administration resulted in mutHTT mRNA and protein reduction with confirmed genomic editing in BACHD mice. Each point represents individual animals with mean±SE. *P<0.05, ****P<0.0001 compared to concurrent naïve animal using unpaired Students t-test.

FIGS. 16A and 16B shows AAV biodistribution and nuclease expression in BACHD mice. FIG. 16A shows AAV5-mediated APG05586 intrastriatal delivery of codon-optimized constructs in BACHD mice resulted in substantial levels of AAV5 vector DNA in clinically relevant brain regions, striatum, and cortex. Disposition was evaluated 6 weeks post administration. Each point represents individual mice with mean SE shown. Naïve and vehicle treated animals are not depicted as they are below LLOQ. FIG. 16B shows nuclease expression from AAV5-mediated APG05586 intrastiatal delivery of codon-optimized constructs in BACHD mice. Disposition was evaluated 6 weeks post administration. Each point represents individual mice with mean±SE shown. Naïve and vehicle treated animals are not depicted as they are below LLOQ.

FIG. 17 shows AAV5 cassettes expressing SGN004282 driven by hU6 (249-318 bp) promoter and mammalian codon-optimized APG05586, directed by various promoters (Jet, hSyn, CMVeb, EFS) reduced mutant huntingtin protein following intrastriatal administration in BACHD mice. Each point represents individual mice with mean±SE shown. *P<0.05, **P<0.01, ****P<0.0001, one-way ANOVA with post-hoc Dunnett's evaluation.

FIG. 18 shows confirmation of editing with NGS INDEL analysis. Genomic editing confirmed with percent INDEL events in the striata and cortex following intrastriatal administration AAV5 cassettes expressing SGN004282 driven by hU6 (249-318 bp) promoter and mammalian codon-optimized APG05586, directed by various promoters (Jet, hSyn, CMVeb, EFS) in BACHD mice. Each point represents individual mice with mean±SE depicted.

FIGS. 19A-19C provide results of introducing increasing amounts of AAV5-hU6-SGN004282-hSyn-APG05586mco-SV40pA that has a mammalian codon optimized APG05586 into BACHD mice after 6 weeks. FIG. 19A provides the vector biodistribution in the striatum and cortex with increasing viral dose.

FIG. 19B shows mutHTT protein reduction in the striatum with increasing viral dose. FIG. 19C shows the dose escalation study of FIG. 19B as percent mutHTT reduction. Additionally, comparison of the dose of 2.7e11 viral genomes (vg) on the left side of the graph in FIG. 19C and the same dose of Optimized Study #2 shows the effect of codon optimization of APG05586. Finally, administration of 7.32e10 vg of the codon optimized construct shows reproducibility of the effect on mutHTT protein levels.

FIGS. 20A-20E demonstrate the specificity of the capillary electrophoresis (CE) immunoassay of mutHTT and wild type HTT. FIGS. 20A and 20B provide the electropherogram of SDS sample blank with FIG. 20A showing the full scale and FIG. 20B the zoomed in view. FIGS. 20C and 20D provide the electropherogram of Q73HTT (mutHTT) or Q7HTT (wild type HTT), respectively, at various concentrations (0.12 ng/ml to 30 ng/ml). FIG. 20E shows the electropherograms of Q73HTT and Q7HTT from FIGS. 20C and 20D, respectively, as a Western blot.

FIGS. 21A and 21B demonstrate the specificity of the CE Immunoassay of mutHTT (Q73HTT) and wild type HTT (Q7HTT) proteins. FIG. 21A provides a graph displaying the Q7HTT linearity and FIG. 21B provides a graph displaying the Q73HTT linearity.

FIGS. 22A and 22B show CE Immunoassay results of brain samples from BACHD mice that have been treated with an AAV5 construct comprising SGN004282 and a codon-optimized APG05586 compared to an untreated animal. FIG. 22A provides the Western Blot and FIG. 22B provides the electropherogram.

FIGS. 23A and 23B show CE Immunoassay results of brain samples from BACHD mice that have been treated with CMV (Treatment 1 and Cohort A) or EFS (Treatment 2 and Cohort B) or untreated mice (naïve or Cohort G). FIG. 23A demonstrates a reduction in mutHTT in the treated mice as compared to the naïve mice and FIG. 23B provides the electropherogram from these studies.

FIGS. 24A-24F provide results in the striatum after the intrastriatal introduction of increasing amounts of AAV5-hU6-SGN004282-CMVeb-APG05586mco-SV40pA or AAV5-hU6-SGN004282-EFS-APG05586mco-bGHpA, each of which has a mammalian codon optimized APG05586, into BACHD mice after 12 weeks. The low, mid, and high doses of AAV5-hU6-SGN004282-CMVeb-APG05586mco-SV40pA were 2.05E10 vg, 7.28E10 vg, and 2.94E11 vg, respectively. The low, mid, and high doses of AAV5-hU6-SGN004282-EFS-APG05586mco-bGHpA were 2.05E10 vg, 7.28E10 vg, and 2.05E11 vg, respectively. FIGS. 24A, 24B, 24C, and 24D provide the biodistribution of the vector, guide RNA, APG05586 mRNA, and APG05586 protein, respectively, in the striatum with increasing viral dose. FIG. 24E shows percent INDEL formation in the striatum with increasing viral dose. FIG. 24F shows mutHTT protein reduction in the striatum with increasing viral dose. In each of FIGS. 24A-24F, results for AAV5-hU6-SGN004282-CMVeb-APG05586mco-SV40pA are shown on the left hand side of the graph and results for AAV5-hU6-SGN004282-EFS-APG05586mco-bGHpA are on the right hand side.

FIGS. 25A-25F provides results in the cortex after the intrastriatal introduction of increasing amounts of AAV5-hU6-SGN004282-CMVeb-APG05586mco-SV40pA or AAV5-hU6-SGN004282-EFS-APG05586mco-bGHpA, each of which has a mammalian codon optimized APG05586, into BACHD mice after 12 weeks. The low, mid, and high doses of AAV5-hU6-SGN004282-CMVeb-APG05586mco-SV40pA were 2.05E10 vg, 7.28E10 vg, and 2.94E11 vg, respectively. The low, mid, and high doses of AAV5-hU6-SGN004282-EFS-APG05586mco-bGHpA were 2.05E10 vg, 7.28E10 vg, and 2.05E11 vg, respectively. FIGS. 25A, 25B, 25C, and 25D provide the biodistribution of the vector, guide RNA, APG05586 mRNA, and APG05586 protein, respectively, in the cortex with increasing viral dose. FIG. 25E shows mutHTT protein reduction in the cortex with increasing viral dose. FIG. 25F shows percent INDEL formation in the cortex with increasing viral dose. In each of FIGS. 25A-25F, results for AAV5-hU6-SGN004282-CMVeb-APG05586mco-SV40pA are shown on the left hand side of the graph and results for AAV5-hU6-SGN004282-EFS-APG05586mco-bGHpA are on the right hand side.

FIGS. 26A-26E provide results in the striata of animals treated with SEQ ID NO:36, SEQ ID NO: 121, SEQ ID NO: 122, or SEQ ID NO: 123 6 weeks after intrastriatal administration of the test article.

FIGS. 26A-26C provide the biodistribution of the vector, APG05586mco mRNA, and guide RNA, respectively. FIG. 26D shows percent INDEL formation and FIG. 26E shows mutHTT protein reduction.

FIGS. 27A-27E show improved activity of AAV constructs with c-MYC NLSs and NLS linker proteins in generating INDELs in HEK293t cells and iPSC-derived astrocytes with AAV5 or AAV6 serotypes. FIG. 27A provides immunofluorescence images of iPSC-derived astrocytes. FIG. 27B shows flow cytometry analysis of iPSC-derived astrocytes stained with an anti-glial fibrillary acidic protein (GFAP)-488 antibody. FIGS. 27C-27E show INDEL rates following AAV6 (FIGS. 27C and 27D) or AAV5 (FIG. 27E) transduction of iPSC-derived astrocytes (FIG. 27C) or HEK293t cells (FIGS. 27D and 27E) with SEQ ID NO: 36, 121, 122, or 123.

FIG. 28 shows the biodistribution of AAV5-packaged pAAV-hU6(249 bp)-SGN004282-CMVeb-c-MYC-NLS-APG05586mco-c-MYC-NLS-SV40pA (179 bp) (SEQ ID NO: 123) following bilateral intrastriatal administration in adult cynomolgus monkey. Animals were administered 225 μl/animal (75 μl/caudate+150 μl/putamen). Low dose (N=2); High dose (N=3). The vector genome was determined by qPCR with primer probe set targeting the APG05586mco sequence.

FIG. 29 shows the expression of APG05586mco in the brain following bilateral intrastriatal administration of AAV5-packaged pAAV-hU6(249 bp)-SGN004282-CMVeb-c-MYC-NLS-APG05586mco-c-MYC-NLS-SV40pA(179 bp) (SEQ ID NO: 123) in adult cynomolgus monkey. Animals were administered 225 μl/animal (75 μl/caudate+150 μl/putamen). Low dose (N=2); High dose (N=3). The mRNA transcripts were quantified by qPCR with primer probe set targeting the APG05586mco sequence.

FIG. 30 shows the expression of SGN004282 guide RNA in the brain following bilateral intrastriatal administration of AAV5-packaged pAAV-hU6(249 bp)-SGN004282-CMVeb-c-MYC-NLS-APG05586mco-c-MYC-NLS-SV40pA(179 bp) (SEQ ID NO: 123) in adult cynomolgus monkey. Animals were administered 225 μl/animal (75 μl/caudate+150 μl/putamen). Low dose (N=2); High dose (N=3). The mRNA transcripts were quantified by qPCR with primer probe set targeting the SGN004282 sequence.

FIG. 31 shows the biodistribution of AAV5-packaged pAAV-hU6(249 bp)-SGN004282-CMVeb-c-MYC-NLS-APG05586mco-c-MYC-NLS-SV40pA(179 bp) (SEQ ID NO: 123) in peripheral tissue following bilateral intrastriatal administration in adult cynomolgus monkey. Animals were administered 225 μl/animal (75 μl/caudate+150 μl/putamen). Low dose (N=2); High dose (N=3). The sample panel was used as suggested in ICH S12 Guideline: Nonclinical Biodistribution Considerations for Gene Therapy Products. After administration of the low dose, 3 of 12 tissues have vector DNA; after administration of the high dose, 6 of 12 tissues have vector DNA. There is no evidence of vector DNA present in Testis/Ovary. *: no evidence of vector DNA present.

FIGS. 32A-32D show a scheme for immunogenicity testing for assaying samples obtained from cynomolgus monkey before and after administration of AAV5-packaged pAAV-hU6(249 bp)-SGN004282-CMVeb-c-MYC-NLS-APG05586mco-c-MYC-NLS-SV40pA(179 bp) (SEQ ID NO: 123). CSF=cerebral spinal fluid; PBMC=peripheral blood mononuclear cells; DC=dendritic cells; MHCII=major histocompatibility complex II. IAV=Influenza A virus derived peptide pool. R10=negative control, medium alone. PHA/SEB=Phytohemagglutinin—positive control for T cell stimulation—non specific TCR independent stimulation.

FIG. 33 shows the pre-existing and post-treatment (Day 29) anti-APG05586mco nuclease antibodies measure in the serum of cynomolgus monkeys. No increase in serum antibodies reactive to APG05586mco were observed in cynomolgus monkeys (N=6) after administration of AAV5-packaged pAAV-hU6(249 bp)-SGN004282-CMVeb-c-MYC-NLS-APG05586mco-c-MYC-NLS-SV40pA(179 bp) (SEQ ID NO: 123).

FIG. 34 shows the levels of anti-APG05586mco total antibodies in the cerebrospinal fluid of cynomolgus monkeys after administration of AAV5-packaged pAAV-hU6(249 bp)-SGN004282-CMVeb-c-MYC-NLS-APG05586mco-c-MYC-NLS-SV40pA(179 bp) (SEQ ID NO: 123).

FIGS. 35A-35D show dose-dependent distribution, nuclease transgene expression and mutHTT protein reduction in a clinically relevant HD murine model. Four weeks following intrastriatal administration of vehicle or AAV5-packaged pAAV-hU6(249 bp)-SGN004282-CMVeb-c-MYC-NLS-APG05586mco-c-MYC-NLS-SV40pA(179 bp) (SEQ ID NO: 123) to BACHD mice, striatal tissues were harvested, and bulk lysate tissue samples assess for AAV vector, nuclease transgene expression (mRNA and protein) and muHTT protein reduction. Each point represents mean±SE with 4 to 6 animals per dose evaluation. Vehicle treated animals were below the LLOQ for the vector and transgene assays with percent muHTT protein reduction being zero.

### DETAILED DESCRIPTION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended embodiments. Although specific terms are employed herein, they are used in a generic and descriptive sense only and not for purposes of limitation.

### I. Overview

HD is an autosomal dominant disease resulting in progressive degeneration of nervous tissue in the brain. Huntington's disease is the result of an expanded trinucleotide repeat in the HTT gene, whereby a significant increase in repeats of a three nucleotide (cytosine-adenine-guanine; CAG) motif in the gene results in a polyglutamine (polyQ) tract in the mutant HTT protein that disrupts the function of the wild-type huntingtin protein.

The presently disclosed compositions and methods take advantage of single nucleotide polymorphisms (SNPs) in the mutant huntingtin (mutHTT) allele that generate a protospacer adjacent motif that allows for the cleavage of the mutHTT allele by an RNA-guided nuclease (RGN). Without being bound by theory, editing of the mutHTT allele can create frameshift INDELs that introduce premature stop codons leading to degradation by non-sense mediated decay and thereby knocking out the full length mutHTT gene. Wild type huntingtin has been shown to support critical cellular and neural functions, thus the selective strategy of targeting only the disease-associated mutant HTT is favored and being explored in preclinical and clinical settings (O'Regan et al., 2020, Sci. Rep. 10:17269; Tabrizi et al., 2019, N Engl J Med 380:2307-2316). Thus, in some embodiments, the presently disclosed compositions and methods provide an allele-specific approach of targeting only the mutHTT allele and not the wtHTT allele. The presently disclosed allele-specific approach targets cells and patients that are heterozygous for a SNP wherein a SNP allele that is linked with the CAG expansion on the mutHTT allele generates a PAM for an RGN. The introduction of an RGN that recognizes that PAM, along with a guide RNA that targets a sequence adjacent to the PAM, into the cell or patient results in the cleavage of the mutHTT allele at or near the SNP, the introduction of an INDEL (insertion or deletion), resulting in the reduction of mutHTT mRNA and protein levels. Due to the heterozygous nature of the cell or patient at the SNP, only the mutHTT allele will be cleaved and only the mutHTT protein levels will be reduced, leaving the wtHTT allele and protein levels unchanged.

While others have shown editing of mutHTT in HD models, no one has taken a SNP-derived PAM-dependent approach within exon 50 that allows for allele-specific reduction in mutHTT levels. The present disclosure provides, for the first time, a single AAV-delivered construct containing both an RNA-guided nuclease and gRNAs that target exon 50 of the mutant allele of HTT in a SNP-derived, PAM-dependent approach, wherein the construct is delivered in vivo to both the striatum and the cortex, two regions known to be important to the pathogenesis of Huntington's Disease. Importantly, this construct demonstrates allele-specific reduction of mutant HTT mRNA and protein in vitro and in vivo. Notably, the presently disclosed approach allows for a reduction in mutHTT mRNA and protein levels of at least 40% for at least 12 weeks after treatment.

The presently disclosed compositions and methods can thus be used for the treatment of HD in subjects in need thereof by reducing HTT levels, and in some embodiments, this reduction is allele-specific wherein only mutHTT levels are reduced and the expression of wild-type HTT (wtHTT) is unchanged. In some embodiments, the presently disclosed compositions and methods can reduce mutHTT mRNA and protein levels at least 40% in at least 50% of striatal neurons.

### II. Huntingtin (HTT) Gene

Huntington's Disease (HD) is an inherited autosomal dominant disease characterized by progressive degeneration of nerve cells in the brain caused by an expansion of a CAG repeat in the first exon of the huntingtin gene on chromosome 4 (Huntington's Disease Collaborative Research Group, 1993, Cell 72:971-983). Disruption of the wild-type HTT protein by the polyglutamine-containing mutant HTT protein results in neural stress and malfunction, ultimately causing striatum atrophy, cognitive impairment, progressive psychiatric and motor deficits (Kaemmerer et al, 2019, Degenerative Neurological and Neuromuscular Disease 9:3-17; Ross et al., 2014, Nat. Rev. Neurol. 10:204-216).

The huntingtin gene is large, spanning 180 kb and consisting of 67 exons. A non-limiting example of a HTT gene is the human HTT gene set forth as NCBI Gene ID No. 3064 and a non-limiting example of a HTT protein is the human huntingtin protein set forth as NCBI Reference Sequence ID No. NP_001375421.1 and herein as SEQ ID NO: 85 (both of which are incorporated by reference herein), which comprises 21 glutamines in the polyQ tract and matches the GRCh38 reference genome.

The CAG triplet repeat region is within exon 1 of the HTT gene and individuals with more than 26 CAG repeats have a greater likelihood of passing on an expanded CAG repeat to their children. HTT genes with 27-35 CAG repeats are considered to be intermediate alleles with an approximately 0% likelihood of developing a disease phenotype, but individuals with these intermediate alleles can pass on the expanded repeats to their offspring. People with 36-39 CAG repeats have a higher likelihood of developing disease symptoms, but the alleles are considered incompletely penetrant. Huntington's disease patients have 40 or more CAG repeats and about 100% likelihood of developing disease symptoms. Those HD patients with 56 or more CAG repeats typically have an earlier onset of the disease in their childhood or teenage years, which is classified as Juvenile Huntington's disease or Juvenile Onset Huntington's disease (JHD) (Tabrizi et al., 2022, Lancet Neurol 21:632-644). Thus, in some embodiments, the cell that is modified with the presently disclosed compositions and methods have an HTT gene with at least 27 CAG repeats within the CAG repeat region in exon 1, which is referred to herein as a mutant HTT gene or allele or mutHTT gene or allele. A CAG repeat region with at least 27 CAG repeats is also referred to herein as a CAG repeat expansion. A wild-type HTT gene or allele or wtHTT gene or allele has less than 27 CAG repeats and typically 15-20 CAG repeats in exon 1. The subjects that are treated with the presently disclosed compositions and methods have an HTT gene with at least 36 CAG repeats and in some embodiments, at least 40 CAG repeats. Most HD patients are heterozygous for the expanded CAG repeat and thus carry one mutHTT allele with at least 36 or at least 40 CAG repeats, and one wtHTT allele with less than 27 CAG repeats.

According to the invention, the cell or subject further comprises a single nucleotide polymorphism (SNP) allele within the mutHTT allele, which can be either a major allele (present in the majority of the human population) or minor allele (present in a minority of the population). It should be noted that it is possible for a particular genomic location to have multiple SNP minor alleles. As used herein, a “single nucleotide polymorphism allele” or “SNP allele” refers to a single nucleotide difference between members of a population at a particular site in the genome, wherein the difference is the substitution of one nucleotide for another. The SNP present on the mutHTT allele generates or is comprised within a PAM that can be recognized by an RGN. Thus, the SNP allele that generates a PAM is linked with the CAG repeat expansion of mutHTT, or in other words, the SNP allele is present on the same copy of the HTT gene as the CAG repeat expansion (the mutHTT allele).

Non-limiting examples of SNPs that can be targeted for an allele-specific approach for treating HD are found in Tables 1 and 2 herein and include NCBI dbSNP No. rs362331. In some embodiments, the SNP allele generates a PAM having the nucleotide sequence of NNNNCC, NNRYA, NNGRR, and/or NNGG. In some embodiments, the method for cleaving a mutant huntingtin (mutHTT) allele in a cell, wherein the mutHTT allele comprises a first single nucleotide polymorphism (SNP) allele in exon 1, and wherein the first SNP allele generates a protospacer adjacent motif (PAM) selected from NNNNCC, NNRYA, NNGRR, and/or NNGG, the method comprises introducing an RNA-guided nuclease (RGN) or a nucleic acid molecule encoding the RGN, and a guide RNA or a nucleic acid molecule encoding the guide RNA, wherein the RGN is capable of recognizing the PAM and cleaving the mutHTT allele. In some of these embodiments, the RGN has at least 80%, 85%, 90%, 95%, or more sequence identity to any one of SEQ ID NOs: 7, 11, 13, and 15. The SNP allele can be within exon 1 on either side of the CAG repeat. In these embodiments, an RGN that recognizes the SNP allele on one side (5′ or 3′) of the CAG repeat can be used in combination with another nuclease that cleaves the opposite end of the CAG repeat to generate an in-frame excision of the CAG repeat region from the mutHTT allele.

In some embodiments, the SNP allele that is linked or “in phase” with the CAG repeat expansion of mutHTT is in exon 50. The rs362331 SNP results in a nucleotide difference at position 151 in exon 50 of the HTT gene, which is set forth herein as SEQ ID NO: 1 and 2. The rs362331 SNP major allele comprises a T or thymine at position 151 in exon 50 (SEQ ID NO: 1) of the HTT gene and the minor allele comprises a C or cytosine at that position (SEQ ID NO: 2). In those genomes wherein the HTT gene comprises the rs362331 SNP “T” allele (the major allele), the presence of the T at position 151 in exon 50 creates a PAM motif (NNRYA) for the RGN APG05586 (set forth as SEQ ID NO: 7) or an active variant or fragment thereof. The RGN APG05586 PAM motif is on the reverse complement of the DNA strand with the T allele, wherein the “A” of the NNRYA PAM is the complementary base of the T allele. Thus, in individuals comprising at least one rs362331 SNP “T” allele, APG05586, along with a corresponding guide RNA that is complementary to a target sequence upstream (5′) of the PAM motif, can bind to and cleave the rs362331 SNP “T” allele.

Alternatively, in those genomes wherein the HTT gene comprises the rs362331 SNP “C” allele (the minor allele), the presence of the C at position 151 in exon 50 (SEQ ID NO: 2) creates a PAM motif for the RGN APG07433.1 (set forth as SEQ ID NO: 3; PAM of NNNNCC), APG01604 (set forth as SEQ ID NO: 11; PAM of NNGRR), and LPG10145 (set forth as SEQ ID NO: 15; PAM of NNGG) or an active variant or fragment of any thereof. Thus, in individuals comprising at least one rs362331 SNP “C” allele, APG07433.1, APG01604, or LPG10145, along with a corresponding guide RNA that is complementary to a target sequence upstream (5′) to the PAM motif, can bind to and cleave the rs362331 SNP “C” allele.

Cleavage of the mutHTT allele near the site of the SNP can generate a frameshift mutation within the HTT gene, leading to early termination and reduction in levels of the mutHTT mRNA and protein as compared to the cell or subjects in the absence of the RGN and its cognate guide RNA.

In most cases, subjects comprising a mutHTT allele are heterozygous for the allele and comprise a mutHTT allele and a wild type HTT (wtHTT) allele. In some embodiments, the PAM that is generated by the SNP allele in the mutHTT is not found in the wild type HTT (wtHTT) allele and thus, the method is allele-specific as the introduced or administered RGN, along with its cognate guide RNA, only cleaves and edits the mutHTT allele and not the wtHTT allele and only levels of mutHTT mRNA and protein are reduced. An RGN polypeptide or RGN system that is not capable of cleaving a wild type HTT allele means that the RGN polypeptide or RGN system is not capable of cleaving a wild type HTT allele at all or cleaves at a negligible level, such that the level of wtHTT mRNA and/or wtHTT protein is insignificantly decreased. An insignificant decrease exists where, for example, the wtHTT can maintain support of critical cellular and neural functions and/or no symptoms of Huntington's disease is present in an in vivo setting (i.e. in a subject heterozygous for the mutHTT allele and administered the RGN polypeptide or RGN system). In some embodiments, the RGN polypeptide or RGN system cleaves at a negligible level, such that the level of wtHTT mRNA and/or wtHTT protein is decreased 5% or less, 4% or less, 3% or less, 2% or less, 1% or less, 0.9% or less, 0.8% or less, 0.7% or less, 0.6% or less, 0.5% or less, 0.4% or less, 0.3% or less, 0.2% or less, or 0.1% or less, as compared to the level of wtHTT mRNA and/or wtHTT protein in vitro or in vivo where an RGN polypeptide or RGN system of the disclosure has not been introduced.

### III. Guide RNA

The present disclosure provides guide RNAs and polynucleotides encoding the same that target an associated RNA-guided nuclease (RGN) to a target nucleotide sequence in a mutant HTT allele. The term “guide RNA” comprises a nucleotide sequence (i.e., a spacer) having sufficient complementarity with a target nucleotide sequence in the mutant HTT allele to hybridize with the target sequence and direct sequence-specific binding of an associated RGN to the target nucleotide sequence. In some embodiments, when the target nucleotide sequence is double-stranded as is the case with DNA, the target nucleotide sequence comprises a non-target strand (which comprises the PAM sequence) and the target strand, which hybridizes with the spacer of the guide RNA. In these embodiments, the guide RNA has sufficient complementarity with the target strand of a double-stranded target sequence (e.g., target DNA sequence in a mutant HTT allele) such that the guide RNA hybridizes with the target strand and directs sequence-specific binding of an associated RGN to the target sequence (e.g., target DNA sequence in a mutant HTT allele). Therefore, in some embodiments, a guide RNA includes a spacer that is identical to the sequence of the non-target strand except that uracil (U) replaces thymine (T) in the guide RNA.

An RGN's respective guide RNA is one or more RNA molecules (generally, one or two), that can bind to the RGN and guide the RGN to bind to a particular target sequence, and in those embodiments wherein the RGN has nickase or nuclease activity, also cleave the target strand and/or the non-target strand.

In general, a guide RNA comprises a CRISPR RNA (crRNA) and a trans-activating CRISPR RNA (tracrRNA), although some RGNs do not require a tracrRNA. Native guide RNAs that comprise both a crRNA and a tracrRNA generally comprise two separate RNA molecules that hybridize to each other through the repeat sequence of the crRNA and the anti-repeat sequence of the tracrRNA. In certain embodiments, the crRNA and tracrRNA are linked together by a multi-nucleotide linker (e.g., a four-nucleotide linker) to form a single guide RNA molecule, wherein the crRNA and the tracrRNA hybridize to each other through the repeat sequence of the crRNA and the anti-repeat sequence of the tracrRNA. Thus, a guide RNA encompasses a single-guide RNA (sgRNA), where the crRNA segment and the tracrRNA segment are located in the same RNA molecule or strand. A guide RNA can include non-naturally occurring guide RNAs that are not found in nature, are chemically modified, comprise crRNA and/or tracrRNA molecules that are not found in nature, and/or comprise sequences not found in the naturally occurring counterpart molecules.

The present invention provides CRISPR RNAs (crRNAs) or polynucleotides encoding CRISPR RNAs that target an associated RGN to a target sequence in a mutant HTT allele. As used herein, the term “crRNA” refers to an RNA molecule or portion thereof that includes a spacer, which is the nucleotide sequence that directly hybridizes with the target strand of a target sequence, and a CRISPR repeat that comprises a nucleotide sequence that forms a structure, either on its own or in concert with a hybridized tracrRNA, that is recognized by the RGN molecule. As used herein, the term “tracrRNA” or “transactivating crRNA” refers to an RNA molecule that comprises an anti-repeat sequence that has sufficient complementarity to hybridize to at least a portion of the CRISPR repeat of a crRNA to form a structure that is recognized by an RGN molecule. In some embodiments, additional secondary structure(s) (e.g., stem-loops) within the tracrRNA molecule is required for binding to an RGN.

A crRNA comprises a spacer and a CRISPR repeat. The “spacer” has a nucleotide sequence that directly hybridizes with the target strand of a target sequence of interest (e.g., target DNA sequence in a mutant HTT allele). The spacer is engineered to have full or partial complementarity with the target strand of a target sequence of interest (e.g., target DNA sequence in a mutant HTT allele). In some embodiments, the spacer can comprise from about 8 nucleotides to about 30 nucleotides, or more. For example, the spacer can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the spacer is 8, 9, 10, 11, 12, 13, 14, 15, 16, 17, 18, 19, 20, 21, 22, 23, 24, 25, 26, 27, 28, 29, 30, or more nucleotides in length. In some embodiments, the spacer is about 10 to about 26 nucleotides in length, or about 12 to about 30 nucleotides in length. In some embodiments, the spacer is about 30 nucleotides in length. In some embodiments, the spacer is 30 nucleotides in length. In some embodiments, the degree of complementarity between a spacer and the target strand of a target sequence (e.g., target DNA sequence in a mutant HTT allele), when optimally aligned using a suitable alignment algorithm, is between 50% and 99% or more, including but not limited to about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In embodiments, the degree of complementarity between a spacer and the target strand of a target sequence (e.g., target DNA sequence in a mutant HTT allele), when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more. In some embodiments, the spacer can be identical in sequence to the non-target strand of a target sequence. In some of those embodiments wherein the target sequence is a target DNA sequence, the spacer can be identical in sequence to the non-target strand of the target DNA sequence, with the exception of the thymines (Ts) in the non-target strand being replaced by uracils (Us) in the spacer. In embodiments, the spacer is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106(1):23-24).

In some embodiments, a spacer of the disclosure has the nucleotide sequence set forth as SEQ ID NO: 80 or that differs from SEQ ID NO: 80 by 1 or 2 nucleotides. In some embodiments, a spacer has a nucleotide sequence that differs from SEQ ID NO: 80 by 2 nucleotides. In some embodiments, a spacer has a nucleotide sequence that differs from SEQ ID NO: 80 by 1 nucleotide. In some embodiments, a spacer has the nucleotide sequence set forth as SEQ ID NO: 80. In some embodiments, a spacer of the disclosure has the nucleotide sequence set forth as SEQ ID NO: 81 or that differs from SEQ ID NO: 81 by 1 or 2 nucleotides. In some embodiments, a spacer has a nucleotide sequence that differs from SEQ ID NO: 81 by 2 nucleotides. In some embodiments, a spacer has a nucleotide sequence that differs from SEQ ID NO: 81 by 1 nucleotide. In some embodiments, a spacer has the nucleotide sequence set forth as SEQ ID NO: 81. In some embodiments, a spacer of the disclosure has the nucleotide sequence set forth as SEQ ID NO: 82 or that differs from SEQ ID NO: 82 by 1 or 2 nucleotides. In some embodiments, a spacer has a nucleotide sequence that differs from SEQ ID NO: 82 by 2 nucleotides. In some embodiments, a spacer has a nucleotide sequence that differs from SEQ ID NO: 82 by 1 nucleotide. In some embodiments, a spacer has the nucleotide sequence set forth as SEQ ID NO: 82. In some embodiments, a spacer of the disclosure has the nucleotide sequence set forth as SEQ ID NO: 83 or that differs from SEQ ID NO: 83 by 1 or 2 nucleotides. In some embodiments, a spacer has a nucleotide sequence that differs from SEQ ID NO: 83 by 2 nucleotides. In some embodiments, a spacer has a nucleotide sequence that differs from SEQ ID NO: 83 by 1 nucleotide. In some embodiments, a spacer has the nucleotide sequence set forth as SEQ ID NO: 83. In some embodiments, a spacer of the disclosure has the nucleotide sequence set forth as SEQ ID NO: 84 or that differs from SEQ ID NO: 84 by 1 or 2 nucleotides. In some embodiments, a spacer has a nucleotide sequence that differs from SEQ ID NO: 84 by 2 nucleotides. In some embodiments, a spacer has a nucleotide sequence that differs from SEQ ID NO: 84 by 1 nucleotide. In some embodiments, a spacer has the nucleotide sequence set forth as SEQ ID NO: 84.

Along with a spacer, a crRNA further comprises a CRISPR RNA (crRNA) repeat. The CRISPR RNA repeat comprises a nucleotide sequence that forms a structure, either on its own or in concert with a hybridized tracrRNA, that is recognized by the RGN molecule. In some embodiments, the CRISPR RNA repeat can comprise from about 8 nucleotides to about 30 nucleotides, or more. For example, the CRISPR repeat can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the CRISPR repeat is 8, 9, 10, 11, 12, 13, 14, 15, 16, 17, 18, 19, 20, 21, 22, 23, 24, 25, 26, 27, 28, 29, 30, or more nucleotides in length. In some embodiments, the degree of complementarity between a CRISPR repeat and its corresponding tracrRNA antirepeat, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the degree of complementarity between a CRISPR repeat and its corresponding tracrRNA antirepeat, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more.

In some embodiments, the CRISPR repeat comprises the nucleotide sequence of any one of SEQ ID NOs: 4, 8, 12, or 16, or an active variant or fragment thereof, that when comprised within a guide RNA, is capable of directing the sequence-specific binding of an associated RNA-guided nuclease provided herein to a presently disclosed target DNA sequence within a mutant HTT allele. In some embodiments, an active CRISPR repeat variant comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to a nucleotide sequence set forth as any one of SEQ ID NOs: 4, 8, 12, 16, and 106. In some embodiments, an active CRISPR repeat fragment comprises at least 5, 6, 7, 8, 9, 10, 11, 12, 13, 14, 15, 16, 17, 18, 19, 20, 21, or 22 contiguous nucleotides of a nucleotide sequence set forth as any one of SEQ ID NOs: 4, 8, 12, 16, and 106. In some embodiments, the CRISPR repeat comprises a nucleotide sequence that differs from SEQ ID NO: 4 by 1 or 2 nucleotides. In some embodiments, the CRISPR repeat comprises a nucleotide sequence that differs from SEQ ID NO: 4 by 2 nucleotides. In some embodiments, the CRISPR repeat comprises a nucleotide sequence that differs from SEQ ID NO: 4 by 1 nucleotide. In some embodiments, the CRISPR repeat comprises a nucleotide sequence set forth as SEQ ID NO: 4. In some embodiments, the CRISPR repeat comprises a nucleotide sequence that differs from SEQ ID NO: 8 by 1 or 2 nucleotides. In some embodiments, the CRISPR repeat comprises a nucleotide sequence that differs from SEQ ID NO: 8 by 2 nucleotides. In some embodiments, the CRISPR repeat comprises a nucleotide sequence that differs from SEQ ID NO: 8 by 1 nucleotide. In some embodiments, the CRISPR repeat comprises a nucleotide sequence set forth as SEQ ID NO: 8. In some embodiments, the CRISPR repeat comprises a nucleotide sequence that differs from SEQ ID NO: 12 by 1 or 2 nucleotides. In some embodiments, the CRISPR repeat comprises a nucleotide sequence that differs from SEQ ID NO: 12 by 2 nucleotides. In some embodiments, the CRISPR repeat comprises a nucleotide sequence that differs from SEQ ID NO: 12 by 1 nucleotide. In some embodiments, the CRISPR repeat comprises a nucleotide sequence set forth as SEQ ID NO: 12. In some embodiments, the CRISPR repeat comprises a nucleotide sequence that differs from SEQ ID NO: 16 by 1 or 2 nucleotides. In some embodiments, the CRISPR repeat comprises a nucleotide sequence that differs from SEQ ID NO: 16 by 2 nucleotides. In some embodiments, the CRISPR repeat comprises a nucleotide sequence that differs from SEQ ID NO: 16 by 1 nucleotide. In some embodiments, the CRISPR repeat comprises a nucleotide sequence set forth as SEQ ID NO: 16. In some embodiments, the CRISPR repeat comprises a nucleotide sequence that differs from SEQ ID NO: 106 by 1 or 2 nucleotides. In some embodiments, the CRISPR repeat comprises a nucleotide sequence that differs from SEQ ID NO: 106 by 2 nucleotides. In some embodiments, the CRISPR repeat comprises a nucleotide sequence that differs from SEQ ID NO: 106 by 1 nucleotide. In some embodiments, the CRISPR repeat comprises a nucleotide sequence set forth as SEQ ID NO: 106.

In some embodiments, the crRNA is an engineered sequence that is not naturally occurring. In some embodiments, the specific CRISPR repeat is not linked to the engineered spacer in nature and the CRISPR repeat is considered heterologous to the spacer. In some embodiments, the spacer is an engineered sequence that is not naturally occurring.

Presently disclosed guide RNAs comprise a crRNA and a trans-activating CRISPR RNA (tracrRNA), while some presently disclosed compositions and methods utilize RGN polypeptides that do not require a tracrRNA. A tracrRNA molecule comprises a nucleotide sequence comprising a region, referred to herein as the anti-repeat, that has sufficient complementarity to hybridize to a CRISPR repeat of a crRNA. In some embodiments, the tracrRNA molecule further comprises a region with secondary structure (e.g., stem-loop) or forms secondary structure upon hybridizing with its corresponding crRNA. In embodiments, the region of the tracrRNA that is fully or partially complementary to a CRISPR repeat is at the 5′ end of the molecule and the 3′ end of the tracrRNA comprises secondary structure. This region of secondary structure generally comprises several hairpin structures, including the nexus hairpin, which is found adjacent to the anti-repeat. The nexus forms the core of the interactions between the guide RNA and the RGN, and is at the intersection between the guide RNA, the RGN, and the target sequence. The nexus hairpin often has a conserved nucleotide sequence in the base of the hairpin stem, with the motif UNANNC found in many nexus hairpins in tracrRNAs. In some embodiments, guide RNAs or RGN systems of the disclosure use tracrRNAs that comprise non-canonical sequences in the base of the hairpin stem of their nexus hairpins, including UNANNG and CNANNC. In some embodiments, a guide RNA or RGN system of the disclosure uses a tracrRNA that includes, in the base of the nexus hairpin stem, the non-canonical sequence UNANNG or CNANNC. There are often terminal hairpins at the 3′ end of the tracrRNA that can vary in structure and number, but often comprise a GC-rich Rho-independent transcriptional terminator hairpin followed by a string of U's at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

In some embodiments, the anti-repeat of the tracrRNA that is fully or partially complementary to the CRISPR repeat comprises from about 8 nucleotides to about 30 nucleotides, or more. For example, the region of base pairing between the tracrRNA anti-repeat and the CRISPR repeat can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the region of base pairing between the tracrRNA anti-repeat and the CRISPR repeat is 6, 7, 8, 9, 10, 11, 12, 13, 14, 15, 16, 17, 18, 19, 20, 21, 22, 23, 24, 25, 26, 27, 28, 29, 30, or more nucleotides in length. In some embodiments, the degree of complementarity between a CRISPR repeat and its corresponding tracrRNA anti-repeat, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In some embodiments, the degree of complementarity between a CRISPR repeat and its corresponding tracrRNA anti-repeat, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more.

In some embodiments, the entire tracrRNA can comprise from about 60 nucleotides to more than about 210 nucleotides. For example, the tracrRNA can be about 60, about 65, about 70, about 75, about 80, about 85, about 90, about 95, about 100, about 105, about 110, about 115, about 120, about 125, about 130, about 135, about 140, about 150, about 160, about 170, about 180, about 190, about 200, about 210, or more nucleotides in length. In some embodiments, the tracrRNA is 60, 65, 70, 75, 80, 85, 90, 95, 100, 105, 110, 115, 120, 125, 130, 135, 140, 150, 160, 170, 180, 190, 200, 210 or more nucleotides in length. In some embodiments, the tracrRNA is about 70 to about 105 nucleotides in length, including about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, and about 105 nucleotides in length. In embodiments, the tracrRNA is 70 to 105 nucleotides in length, including 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, and 105 nucleotides in length.

In some embodiments, the tracrRNA comprises the nucleotide sequence of any one of SEQ ID NOs: 5, 9, 13, 17, 107, and 120, or an active variant or fragment thereof that when comprised within a guide RNA is capable of directing the sequence-specific binding of an associated RNA-guided nuclease provided herein to a target sequence within a mutant HTT allele. In some embodiments, an active tracrRNA sequence variant comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to any one of the nucleotide sequences set forth as SEQ ID NOs: 5, 9, 13, 17, 107, and 120. In some embodiments, an active tracrRNA sequence fragment comprises at least 5, 10, 15, 20, 25, 30, 35, 40, 45, 50, 55, 60, 65, 70, 75, 80, or more contiguous nucleotides of any one of the nucleotide sequences set forth as SEQ ID NOs: 5, 9, 13, 17, 107, and 120. In some embodiments, an active tracrRNA sequence fragment comprises at least 5, 10, 15, 20, 25, 30, 35, 40, 45, 50, 55, 60, 65, 70, 75, 80, or more contiguous nucleotides of the nucleotide sequence set forth as SEQ ID NO: 5. In certain embodiments, an active tracrRNA sequence fragment comprises at least 5, 10, 15, 20, 25, 30, 35, 40, 45, 50, 55, 60, 65, 70, 75, 80, or more contiguous nucleotides of the nucleotide sequence set forth as SEQ ID NO: 9. In certain embodiments, an active tracrRNA sequence fragment comprises at least 5, 10, 15, 20, 25, 30, 35, 40, 45, 50, 55, 60, 65, 70, 75, 80, or more contiguous nucleotides of the nucleotide sequence set forth as SEQ ID NO: 13. In certain embodiments, an active tracrRNA sequence fragment comprises at least 5, 10, 15, 20, 25, 30, 35, 40, 45, 50, 55, 60, 65, 70, 75, 80, or more contiguous nucleotides of the nucleotide sequence set forth as SEQ ID NO: 17. In certain embodiments, an active tracrRNA sequence fragment comprises at least 5, 10, 15, 20, 25, 30, 35, 40, 45, 50, 55, 60, 65, 70, 75, 80, or more contiguous nucleotides of the nucleotide sequence set forth as SEQ ID NO: 107. In certain embodiments, an active tracrRNA sequence fragment comprises at least 5, 10, 15, 20, 25, 30, 35, 40, 45, 50, 55, 60, 65, 70, 75, 80, or more contiguous nucleotides of the nucleotide sequence set forth as SEQ ID NO: 120. In some embodiments, an active tracrRNA sequence fragment comprises the nucleotide sequence set forth as SEQ ID NO: 5. In some embodiments, an active tracrRNA sequence fragment comprises the nucleotide sequence set forth as SEQ ID NO: 9. In some embodiments, an active tracrRNA sequence fragment comprises the nucleotide sequence set forth as SEQ ID NO: 13. In some embodiments, an active tracrRNA sequence fragment comprises the nucleotide sequence set forth as SEQ ID NO: 17. In some embodiments, an active tracrRNA sequence fragment comprises the nucleotide sequence set forth as SEQ ID NO: 107. In some embodiments, an active tracrRNA sequence fragment comprises the nucleotide sequence set forth as SEQ ID NO: 120.

Two polynucleotide sequences can be considered to be substantially complementary when the two sequences hybridize to each other under stringent conditions. Likewise, an RGN is considered to bind to a particular target sequence in a sequence-specific manner if the guide RNA bound to the RGN binds to a target sequence under stringent conditions. By “stringent conditions” or “stringent hybridization conditions” is intended conditions under which the two polynucleotide sequences will hybridize to each other to a detectably greater degree than to other sequences (e.g., at least 2-fold over background). Stringent conditions are sequence-dependent and will be different in different circumstances. Typically, stringent conditions will be those in which the salt concentration is less than about 1.5 M Na+ ion, typically about 0.01 to 1.0 M Na+ ion concentration (or other salts) at pH 7.0 to 8.3, and the temperature is at least about 30° C. for short sequences (e.g., 10 to 50 nucleotides) and at least about 60° C. for long sequences (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and awash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The Tm is the temperature (under defined ionic strength and pH) at which 50% of a complementary target sequence hybridizes to a perfectly matched sequence. For DNA-DNA hybrids, the Tm can be approximated from the equation of Meinkoth and Wahl (1984) Anal. Biochem. 138:267-284: Tm=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, New York).

The term “sequence specific” can also refer to the binding of a RGN polypeptide to a target sequence at a greater affinity than binding to a randomized background sequence.

The guide RNA can be a single guide RNA (sgRNA) or a dual-guide RNA (dgRNA). A single guide RNA comprises the crRNA and tracrRNA on a single molecule of RNA, whereas a dual-guide RNA system comprises a crRNA and a tracrRNA present on two distinct RNA molecules, hybridized to one another through at least a portion of the CRISPR repeat of the crRNA and at least a portion of the tracrRNA (i.e., the anti repeat), which may be fully or partially complementary to the CRISPR repeat of the crRNA. In embodiments wherein the guide RNA is a single guide RNA, the crRNA and tracrRNA are separated by a linker nucleotide sequence. A crRNA repeat and a tracrRNA linked by a nucleotide linker can be referred to as the backbone of the sgRNA. A backbone can also refer to the crRNA repeat and the tracrRNA of a dgRNA.

A backbone of a guide RNA can comprise the nucleotide sequence of any one of SEQ ID NOs: 140, 141, and 142, or an active variant or fragment thereof, that when comprised within a guide RNA is capable of directing the sequence-specific binding of an associated RNA-guided nuclease provided herein to a target sequence within a mutant HTT allele. A backbone of an sgRNA or dgRNA can be engineered to be shorter or longer than its native length and still retain function. In some embodiments, the backbone of an engineered sgRNA or dgRNA is about 2 to about 30 nucleotides shorter, as compared to the same backbone prior to the engineering. In some embodiments, the backbone of an engineered sgRNA or dgRNA is about 2 nucleotides shorter, about 4 nucleotides shorter, about 6 nucleotides shorter, about 8 nucleotides shorter, about 10 nucleotides shorter, about 12 nucleotides shorter, about 14 nucleotides shorter, about 16 nucleotides shorter, about 18 nucleotides shorter, about 20 nucleotides shorter, about 22 nucleotides shorter, about 24 nucleotides shorter, about 26 nucleotides shorter, about 28 nucleotides shorter, about 30 nucleotides shorter, or more nucleotides shorter as compared to the same backbone prior to the engineering. In some embodiments, the backbone of an engineered sgRNA or dgRNA is about 2 to about 18 nucleotides shorter, as compared to the same backbone prior to the engineering. In some embodiments, the backbone of an engineered sgRNA or dgRNA is about 2 nucleotides shorter, about 4 nucleotides shorter, about 6 nucleotides shorter, about 8 nucleotides shorter, about 10 nucleotides shorter, about 12 nucleotides shorter, about 14 nucleotides shorter, about 16 nucleotides shorter, or about 18 nucleotides shorter, as compared to the same backbone prior to the engineering. In some embodiments, the backbone of an engineered sgRNA or dgRNA is about 14 nucleotides shorter, as compared to the same backbone prior to the engineering. In some embodiments, the backbone of an engineered sgRNA or dgRNA is about 16 nucleotides shorter, as compared to the same backbone prior to the engineering. In some embodiments, the backbone of an engineered sgRNA or dgRNA is about 20 nucleotides shorter, as compared to the same backbone prior to the engineering.

In some embodiments, an active backbone variant of a guide RNA of the disclosure can comprise from about 60 nucleotides to more than about 120 nucleotides. For example, the backbone can be about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, or more nucleotides in length. In some embodiments, an active backbone variant of a guide RNA of the disclosure can comprise from about 66 nucleotides to more than about 110 nucleotides. In some embodiments, an active backbone variant of a guide RNA of the disclosure can comprise 66 nucleotides in length. In some embodiments, an active backbone variant of a guide RNA of the disclosure can comprise 70 nucleotides in length. In some embodiments, an active backbone variant of a guide RNA of the disclosure can comprise 76 nucleotides in length. In some embodiments, an active backbone variant of a guide RNA of the disclosure can comprise 90 nucleotides in length. In some embodiments, an active backbone variant of a guide RNA of the disclosure can comprise 94 nucleotides in length. In some embodiments, an active backbone variant of a guide RNA of the disclosure can comprise 110 nucleotides in length.

An active backbone fragment of a guide RNA of the present disclosure can comprise at least 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, or more contiguous nucleotides of any one of the nucleotide sequences set forth as SEQ ID NO: 140, 141, or 142. In some embodiments, an active backbone fragment of a guide RNA of the disclosure comprises at least 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, or more contiguous nucleotides of the nucleotide sequence set forth as SEQ ID NO: 140. In some embodiments, an active backbone fragment of a guide RNA of the disclosure comprises at least 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of the nucleotide sequence set forth as SEQ ID NO: 141. In some embodiments, an active backbone fragment of a guide RNA of the disclosure comprises at least 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, or more contiguous nucleotides of the nucleotide sequence set forth as SEQ ID NO: 142.

An active backbone variant of a sgRNA of the present disclosure can have at least 40%, 45%, 50%, 55%, 60%, 65%, 70%, 75%, 80%, 85%, 90%, 91%, 92%, 93%, 94%, 95%, 96%, 97%, 98%, 99% or more sequence identity to any one of SEQ ID NOs: 140-142. In some embodiments, an active backbone variant of a sgRNA of the disclosure has a nucleotide sequence having at least 80% sequence identity to any one of SEQ ID NOs: 140-142. In some embodiments, an active backbone variant of a sgRNA of the disclosure has a nucleotide sequence having at least 85% sequence identity to any one of SEQ ID NOs: 140-142. In some embodiments, an active backbone variant of a sgRNA of the disclosure has a nucleotide sequence having at least 90% sequence identity to any one of SEQ ID NOs: 140-142. In some embodiments, an active backbone variant of a sgRNA of the disclosure has a nucleotide sequence having at least 95% sequence identity to any one of SEQ ID NOs: 140-142. In some embodiments, an active backbone variant of a sgRNA of the disclosure has the nucleotide sequence set forth as any one of SEQ ID NOs: 140-142.

In general, the linker nucleotide sequence connecting a crRNA and a tracrRNA is one that does not include complementary bases in order to avoid the formation of secondary structure within or comprising nucleotides of the linker nucleotide sequence. In some embodiments, the linker nucleotide sequence between the crRNA and tracrRNA is at least 3, at least 4, at least 5, at least 6, at least 7, at least 8, at least 9, at least 10, at least 11, at least 12, or more nucleotides in length. In some embodiments, the linker nucleotide sequence of a single guide RNA is at least 4 nucleotides in length. In certain embodiments, the linker nucleotide sequence of a single guide RNA is 4 nucleotides in length. In some embodiments, the linker nucleotide sequence includes a nucleotide sequence set forth as any of AAAG, GAAA, ACUU, and CAAAGG. In certain embodiments, the linker nucleotide sequence includes a nucleotide sequence set forth as AAAG. In some embodiments, the linker nucleotide sequence includes a nucleotide sequence set forth as GAAA. In some embodiments, the linker nucleotide sequence includes a nucleotide sequence set forth as ACUU. In some embodiments, the linker nucleotide sequence includes a nucleotide sequence set forth as CAAAGG.

In some embodiments, a sgRNA has a nucleotide sequence set forth as any one of SEQ ID NOs: 6, 10, 14, 18, and 25-29.

The single guide RNA or dual-guide RNA can be synthesized chemically or via in vitro transcription. Assays for determining sequence-specific binding between an RGN and a guide RNA are known in the art and include, but are not limited to, in vitro binding assays between an expressed RGN and the guide RNA, which can be tagged with a detectable label (e.g., biotin) and used in a pull-down detection assay in which the guide RNA:RGN complex is captured via the detectable label (e.g., with streptavidin beads). A control guide RNA with an unrelated sequence or structure to the guide RNA can be used as a negative control for non-specific binding of the RGN to RNA.

In some embodiments, the guide RNA can be introduced into a target cell as an RNA molecule. The guide RNA can be transcribed in vitro or chemically synthesized. In some embodiments, a nucleic acid molecule encoding the guide RNA is introduced into a target cell. In some embodiments, the nucleic acid molecule encoding the guide RNA is operably linked to a promoter (e.g., an RNA polymerase III promoter). The promoter can be a native promoter or heterologous to the guide RNA-encoding nucleic acid molecule.

In some embodiments, the guide RNA can be introduced into a target cell as part of a ribonucleoprotein complex, as described herein, wherein the guide RNA is bound to an RGN polypeptide.

The guide RNA directs an associated RGN to a particular target nucleotide sequence of interest through hybridization of the guide RNA to the target sequence of interest. The target sequence can be bound (and in some embodiments, cleaved) by an RNA-guided nuclease in vitro or in a cell. A target sequence can comprise DNA, RNA, or a combination of both and can be single-stranded or double-stranded. In some embodiments, a target sequence can be genomic DNA (i.e., chromosomal DNA), plasmid DNA, episomal DNA, or an RNA molecule (e.g., messenger RNA, ribosomal RNA, transfer RNA, microRNA, small interfering RNA). In those embodiments wherein the target sequence is a chromosomal sequence, the chromosomal sequence can be a nuclear or mitochondrial chromosomal sequence. In the presently disclosed compositions and methods, the target sequence is within a target nucleic acid molecule that is double-stranded (e.g., a target DNA sequence). More specifically, the target sequence is within a mutant HTT allele. In some embodiments, the target sequence is unique in the target genome. In some embodiments, the target sequence comprises a target strand and a non-target strand, and the target sequence has the nucleotide sequence set forth as any one of SEQ ID NOs: 75-79, and 130.

The target sequence is adjacent to a protospacer adjacent motif (PAM) and the non-target strand of the target sequence is the strand that comprises the PAM. The PAM is immediately adjacent to the target sequence and often comprises Ns, which represent any nucleotide. In some embodiments, the PAM comprises about 1 to about 10 Ns, including about 1, about 2, about 3, about 4, about 5, about 6, about 7, about 8, about 9, or about 10 Ns. In certain embodiments, a PAM comprises 1 to 10 Ns, including 1, 2, 3, 4, 5, 6, 7, 8, 9, or 10 Ns. The PAM can be 5′ or 3′ of the target sequence on its non-target strand. In some embodiments, the PAM is 3′ of the target sequence on its non-target strand for the presently disclosed guide RNAs and RGN systems. Generally, the PAM is a consensus sequence of about 3-4 nucleotides, but in certain embodiments it can be 2, 3, 4, 5, 6, 7, 8, 9, or more nucleotides in length.

In some embodiments, a PAM sequence adjacent to a presently disclosed target sequence on its non-target strand comprises the consensus sequence set forth as any one of NNNNCC, NNRYA, NNGRR, and NNGG. In some embodiments, a PAM sequence adjacent to a target sequence on its non-target strand includes the consensus sequence set forth as NNNNCC. In some embodiments, a PAM sequence adjacent to a target sequence on its non-target strand includes the consensus sequence set forth as NNRYA. In some embodiments, a PAM sequence adjacent to a target sequence on its non-target strand includes the consensus sequence set forth as NNGRR. In some embodiments, a PAM sequence adjacent to a target sequence on its non-target strand includes the consensus sequence set forth as NNGG. In some embodiments, the PAM sequence is 3′ of the target sequence on its non-target strand.

It is well-known in the art that PAM sequence specificity for a given nuclease enzyme is affected by enzyme concentration (see, e.g., Karvelis et al. (2015) Genome Biol 16:253), which may be modified by altering the promoter used to express the RGN, or the amount of ribonucleoprotein complex delivered to the cell.

Upon recognizing its corresponding PAM sequence, the RGN can cleave one or both strands of a target sequence at a specific cleavage site. As used herein, a cleavage site is made up of the two particular nucleotides within a target sequence between which the target strand and/or the non-target strand of a target sequence is cleaved by an RGN. The cleavage site can comprise the 1^{st} and 2^{nd}, 2^{nd} and 3^{rd}, 3^{rd} and 4^{th}, 4^{th} and5^{th}, 5^{th} and 6^{th}, 7^{th} and 8^{th}, or8^{th} and9^{th} nucleotides from the PAM in either the 5′ or 3′ direction. In some embodiments, the cleavage site may be over 10, 11, 12, 13, 14, 15, 16, 17, 18, 19, or 20 nucleotides from the PAM in either the 5′ or 3′ direction. As RGNs can cleave a target sequence resulting in staggered ends, in certain embodiments, the cleavage site is defined based on the distance of the two nucleotides from the PAM on the non-target strand of the target sequence and, for the target strand, the distance of the two nucleotides from the complement of the PAM.

### IV. RNA-Guided Nucleases and Other Nucleases

In some embodiments of the methods for cleaving the mutHTT allele and treating Huntington's disease, the RGN is a Type II CRISPR-Cas polypeptide. In some embodiments, the RGN is a Type V CRISPR-Cas polypeptide. In some embodiments, the RGN is a Cas9, a CasX, a CasY, a Cpf1, a C2c1, a C2c2, a C2c3, a GeoCas9, a CjCas9, a Cas12a, a Cas12b, a Cas12g, a Cas12h, a Cas12i, a Cas13b, a Cas13c, a Cas13d, a Cas14, a Csn2, an xCas9, an SpCas9-NG, an LbCas12a, an AsCas12a, a Cas9-KKH, a circularly permuted Cas9, an Argonaute (Ago), a SmacCas9, or a Spy-macCas9 domain.

Provided herein are RNA-guided nuclease systems comprising the presently disclosed guide RNAs. The term RNA-guided nuclease (RGN) refers to a polypeptide that is directed to a particular target sequence (e.g., target DNA sequence in a mutant HTT allele) in a sequence-specific manner by binding a guide RNA molecule that hybridizes with the target strand of the target sequence (e.g., target DNA sequence in a mutant HTT allele). Active fragments or variants thereof of naturally-occurring RGNs maintain binding to a target nucleotide sequence in an RNA-guided sequence-specific manner. Cleavage of a target strand of a target sequence by an RGN can result in a single- or double-stranded break, but generally generate a double-stranded break.

The presently disclosed RGN systems comprise an RGN that binds to a target sequence disclosed herein. In some embodiments, the RGN recognizes a PAM having a consensus nucleotide sequence including NNNNCC, NNRYA, NNGRR, and NNGG 3′ of the target sequence on its non-target strand (where N is A, C, T, or G; R is G or A; Y is C or T), and active fragments or variants thereof. In some embodiments, the RGN recognizes a PAM having a consensus nucleotide sequence including a NNNNCC 3′ of the target sequence on its non-target strand (where N is A, C, T, or G), and active fragments or variants thereof. In some embodiments, the RGN recognizes a PAM having a consensus nucleotide sequence including NNRYA 3′ of the target sequence on its non-target strand (where N is A, C, T, or G; R is G or A; Y is C or T), and active fragments or variants thereof. In some embodiments, the RGN recognizes a PAM having a consensus nucleotide sequence including NNGRR 3′ of the target sequence on its non-target strand (where N is A, C, T, or G; R is G or A), and active fragments or variants thereof. In some embodiments, the RGN recognizes a PAM having a consensus nucleotide sequence including NNGG 3′ of the target sequence on its non-target strand (where N is A, C, T, or G), and active fragments or variants thereof. In some embodiments, the active fragment or variant of an RGN recognizing such PAM sequences is capable of binding and in some embodiments, cleaving or nicking a target sequence.

An RGN polypeptide of the present disclosure can comprise a linker domain 1 (L1), a linker domain 2 (L2), a wedge (WED) domain, a RuvC nuclease domain, an HNH nuclease domain, a bridge helix (BH) domain, a Rec domain, or a PAM-interacting (PI) domain. In some embodiments, the RuvC domain is the RuvCIII domain. A Rec or recognition lobe mediates nucleic acid binding through multiple Rec domains (e.g., Rec1-3) by sensing nucleic acids, regulates the HNH conformational transition, and locks the catalytic HNH domain at the cleavage site. A wedge domain is responsible for the recognition of guide RNA scaffolds. An arginine-rich bridge helix (BH) domain connects the nuclease lobe and recognition lobe.

Non-limiting examples of domains within the APG07433.1 RGN polypeptide, set forth as SEQ ID NO: 3, include: RuvC-I from amino acid residues 1-54; BH from amino acid residues 55-83; REC1 from amino acid residues 84-244; REC2 from amino acid residues 245-462; RuvC-II from amino acid residues 463-521; L1 from amino acid residues 522-552; HNH from amino acid residues 553-672; L2 from amino acid residues 673-685; RuvC-III from amino acid residues 686-833; WED from amino acid residues 834-938; and PI from amino acid residues 939-1071, all in reference to SEQ ID NO: 3.

Non-limiting examples of domains within the APG05586 RGN polypeptide, set forth as SEQ ID NO: 7, has the following domains: RuvC-I from amino acid residues 1-33; BH from amino acid residues 34-71; REC1 from amino acid residues 72-232; REC2 from amino acid residues 233-468; RuvC-II from amino acid residues 469-517; L1 from amino acid residues 518-552; HNH from amino acid residues 553-672; L2 from amino acid residues 673-687; RuvC-III from amino acid residues 688-837; WED from amino acid residues 838-998; and PI from amino acid residues 999-1150, all in reference to SEQ ID NO: 7.

Non-limiting examples of domains within the APG01604 RGN polypeptide, set forth as SEQ ID NO: 11, has the following domains: RuvC-I from amino acid residues 1-40; BH from amino acid residues 41-74; REC1 from amino acid residues 75-223; REC2 from amino acid residues 224-430; RuvC-II from amino acid residues 431-483; L1 from amino acid residues 484-516; HNH from amino acid residues 517-631; L2 from amino acid residues 632-651; RuvC-III from amino acid residues 652-775; WED from amino acid residues 776-909; and PI from amino acid residues 910-1052, all in reference to SEQ ID NO: 11.

Non-limiting examples of domains within the LPG10145 RGN polypeptide, set forth as SEQ ID NO: 15, has the following domains: RuvC-I from amino acid residues 1-42; BH from amino acid residues 43-79; REC1 from amino acid residues 80-236; REC2 from amino acid residues 237-476; RuvC-II from amino acid residues 477-524; L1 from amino acid residues 525-560; HNH from amino acid residues 561-676; L2 from amino acid residues 677-690; RuvC-III from amino acid residues 691-828; WED from amino acid residues 829-976; and PI from amino acid residues 977-1130, all in reference to SEQ ID NO: 15.

The presently disclosed RGN systems can include an RGN that comprises a PAM-interacting domain that contributes to the recognition of and binding to a PAM site. In particular embodiments, the PAM-interacting domain of the RGN has the sequence set forth as SEQ ID NO: 133 and recognizes the PAM sequence NNNNCC. For example, an RGN polypeptide having at least 80%, at least 85%, at least 90%, at least 95%, or 100% sequence identity to SEQ ID NO: 3 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 133 and recognizes the PAM sequence NNNNCC. In some embodiments, an RGN polypeptide having at least 80%, at least 85%, at least 90%, at least 95%, or 100% sequence identity to SEQ ID NO: 3 comprises a PAM-interacting domain having an amino acid sequence having at least 95% sequence identity to SEQ ID NO: 133 and recognizes the PAM sequence NNNNCC. In some embodiments, an RGN polypeptide having at least 80%, at least 85%, at least 90%, at least 95%, or 100% sequence identity to SEQ ID NO: 3 comprises a PAM-interacting domain having the sequence set forth as SEQ ID NO: 133 and recognizes the PAM sequence NNNNCC.

In some embodiments, the PAM-interacting domain of the RGN has the sequence set forth as SEQ ID NO: 134 and recognizes the PAM sequence NNRYA. For example, an RGN polypeptide having at least 80%, at least 85%, at least 90%, at least 95%, or 100% sequence identity to SEQ ID NO: 7 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 134 and recognize the PAM sequence NNRYA. In some embodiments, an RGN polypeptide having at least 80%, at least 85%, at least 90%, at least 95%, or 100% sequence identity to SEQ ID NO: 7 comprises a PAM-interacting domain having an amino acid sequence having at least 95% sequence identity to SEQ ID NO: 134 and recognizes the PAM sequence NNRYA. In some embodiments, an RGN polypeptide having at least 80%, at least 85%, at least 90%, at least 95%, or 100% sequence identity to SEQ ID NO: 7 comprises a PAM-interacting domain having the sequence set forth as SEQ ID NO: 134 and recognizes the PAM sequence NNRYA.

In some embodiments, the PAM-interacting domain of the RGN has the sequence set forth as SEQ ID NO: 135 and recognizes the PAM sequence NNGRR. For example, an RGN polypeptide having at least 80%, at least 85%, at least 90%, at least 95%, or 100% sequence identity to SEQ ID NO: 11 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 135 and recognize the PAM sequence NNGRR. In some embodiments, an RGN polypeptide having at least 80%, at least 85%, at least 90%, at least 95%, or 100% sequence identity to SEQ ID NO: 11 comprises a PAM-interacting domain having an amino acid sequence having at least 95% sequence identity to SEQ ID NO: 135 and recognizes the PAM sequence NNGRR. In some embodiments, an RGN polypeptide having at least 80%, at least 85%, at least 90%, at least 95%, or 100% sequence identity to SEQ ID NO: 11 comprises a PAM-interacting domain having the sequence set forth as SEQ ID NO: 135 and recognizes the PAM sequence NNGRR.

In some embodiments, the PAM-interacting domain of the RGN has the sequence set forth as SEQ ID NO: 136 and recognizes the PAM sequence NNGG. For example, an RGN polypeptide having at least 80%, at least 85%, at least 90%, at least 95%, or 100% sequence identity to SEQ ID NO: 15 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 136 and recognize the PAM sequence NNGG. In some embodiments, an RGN polypeptide having at least 80%, at least 85%, at least 90%, at least 95%, or 100% sequence identity to SEQ ID NO: 15 comprises a PAM-interacting domain having an amino acid sequence having at least 95% sequence identity to SEQ ID NO: 136 and recognizes the PAM sequence NNGG. In some embodiments, an RGN polypeptide having at least 80%, at least 85%, at least 90%, at least 95%, or 100% sequence identity to SEQ ID NO: 15 comprises a PAM-interacting domain having the sequence set forth as SEQ ID NO: 136 and recognizes the PAM sequence NNGG. The PAM-interacting domains of the APG07433.1 nuclease (set forth as SEQ ID NO: 3), the APG05586 nuclease (set forth as SEQ ID NO: 7), the APG01604 nuclease (set forth as SEQ ID NO: 11), and the LPG10145 nuclease (set forth as SEQ ID NO: 15) were determined by aligning the nuclease sequences to known RNA-guided nucleases with solved structures, including Staphylococcus aureus (PDB: 5CZZ-Chain-A), Neisseria menigitidis 1 (PDB: 6JDV_1|Chain), and Streptococcus thermophilus (6MOW_4|Chain) and identifying the region of a similar location in the protein alignment.

The presently disclosed RGN systems can include an RGN polypeptide that comprises at least one nuclease domain, each of which is responsible for cleaving a single strand of a nucleic acid molecule. The nuclease domain can comprise a RuvC or an HNH domain. For example, an RGN polypeptide having at least 80%, at least 85%, at least 90%, at least 95%, or 100% sequence identity to SEQ ID NO: 3 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a nuclease domain having an amino acid sequence having at least 95% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a nuclease domain having the sequence set forth as any one of SEQ ID NOs: 143, 144, 145, and 146.

An RGN polypeptide of the present disclosure having at least 80%, at least 85%, at least 90%, at least 95%, or 100% sequence identity to SEQ ID NO: 7 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a nuclease domain having an amino acid sequence having at least 95% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a nuclease domain having the sequence set forth as any one of SEQ ID NOs: 147, 148, 149, and 150.

An RGN polypeptide of the present disclosure having at least 80%, at least 85%, at least 90%, at least 95%, or 100% sequence identity to SEQ ID NO: 11 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a nuclease domain having an amino acid sequence having at least 95% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a nuclease domain having the sequence set forth as any one of SEQ ID NOs: 151, 152, 153, and 154.

An RGN polypeptide having at least 80%, at least 85%, at least 90%, at least 95%, or 100% sequence identity to SEQ ID NO: 15 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a nuclease domain having an amino acid sequence having at least 95% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a nuclease domain having the sequence set forth as any one of SEQ ID NOs: 155, 156, 157, and 158.

The presently disclosed RGN systems can include an RGN polypeptide that comprises a PAM-interacting domain that contributes to the recognition of and binding to a PAM site and further comprises at least one nuclease domain, each of which nuclease domain is responsible for cleaving a single strand of a nucleic acid molecule. For example, an RGN polypeptide having at least 80%, at least 85%, at least 90%, at least 95%, or 100% sequence identity to SEQ ID NO: 3 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 133 and recognizes the PAM sequence NNNNCC, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a PAM-interacting domain having an amino acid sequence having at least 95% sequence identity to SEQ ID NO: 133 and recognizes the PAM sequence NNNNCC, and can further comprise a nuclease domain having an amino acid sequence having at least 95% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a PAM-interacting domain having the sequence set forth as SEQ ID NO: 133 and recognizes the PAM sequence NNNNCC, and can further comprise a nuclease domain having the amino acid sequence set forth as any one of SEQ ID NOs: 143, 144, 145, and 146.

An RGN polypeptide of the present disclosure having at least 80%, at least 85%, at least 90%, at least 95%, or 100% sequence identity to SEQ ID NO: 7 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 134 and recognize the PAM sequence NNRYA, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a PAM-interacting domain having an amino acid sequence having at least 95% sequence identity to SEQ ID NO: 134 and recognizes the PAM sequence NNRYA, and can further comprise a nuclease domain having an amino acid sequence having at least 95% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a PAM-interacting domain having the sequence set forth as SEQ ID NO: 134 and recognizes the PAM sequence NNRYA, and can further comprise a nuclease domain having the amino acid sequence set forth as any one of SEQ ID NOs: 147, 148, 149, and 150.

An RGN polypeptide of the present disclosure having at least 80%, at least 85%, at least 90%, at least 95%, or 100% sequence identity to SEQ ID NO: 11 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 135 and recognize the PAM sequence NNGRR, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a PAM-interacting domain having an amino acid sequence having at least 95% sequence identity to SEQ ID NO: 135 and recognizes the PAM sequence NNGRR, and can further comprise a nuclease domain having an amino acid sequence having at least 95% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a PAM-interacting domain having the sequence set forth as SEQ ID NO: 135 and recognizes the PAM sequence NNGRR, and can further comprise a nuclease domain having the amino acid sequence set forth as any one of SEQ ID NOs: 151, 152, 153, and 154.

An RGN polypeptide of the present disclosure having at least 80%, at least 85%, at least 90%, at least 95%, or 100% sequence identity to SEQ ID NO: 15 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 136 and recognize the PAM sequence NNGG, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a PAM-interacting domain having an amino acid sequence having at least 95% sequence identity to SEQ ID NO: 136 and recognizes the PAM sequence NNGG, and can further comprise a nuclease domain having an amino acid sequence having at least 95% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a PAM-interacting domain having the sequence set forth as SEQ ID NO: 136 and recognizes the PAM sequence NNGG, and can further comprise a nuclease domain having the amino acid sequence set forth as any one of SEQ ID NOs: 155, 156, 157, and 158.

In some embodiments, an RGN, or an active variant or fragment thereof, capable of binding a target sequence adjacent to a PAM consensus sequence (i.e., capable of recognizing the PAM consensus sequence) set forth as any one of NNNNCC, NNRYA, NNGRR, and NNGG is used in the presently disclosed compositions and methods. In some embodiments, the PAM sequence is 3′ of the target sequence on its non-target strand. In some embodiments, the RGN binds to a guide RNA having a sequence set forth as any one of SEQ ID NOs: 6, 10, 14, 18, and 25-29. In some embodiments, an RGN having at least 90% sequence identity to the amino acid sequence set forth as SEQ ID NO: 3 is capable of recognizing a PAM sequence of NNNNCC, and binds to a guide RNA comprising: a CRISPR repeat set forth as SEQ ID NO: 4, or an active variant or fragment thereof; and a tracrRNA set forth as SEQ ID NO: 5, or an active variant or fragment thereof. In some embodiments, an RGN having at least 90% sequence identity to the amino acid sequence set forth as SEQ ID NO: 7 is capable of recognizing a PAM sequence of NNRYA, and binds to a guide RNA comprising: a CRISPR repeat set forth as SEQ ID NO: 8 or 106, or an active variant or fragment thereof; and a tracrRNA set forth as SEQ ID NO: 9 or 107, or an active variant or fragment thereof. In some embodiments, an RGN having at least 90% sequence identity to the amino acid sequence set forth as SEQ ID NO: 11 is capable of recognizing a PAM sequence of NNGRR, and binds to a guide RNA comprising: a CRISPR repeat set forth as SEQ ID NO: 12, or an active variant or fragment thereof; and a tracrRNA set forth as SEQ ID NO: 13 or 120, or an active variant or fragment thereof. In some embodiments, an RGN having at least 90% sequence identity to the amino acid sequence set forth as SEQ ID NO: 15 is capable of recognizing a PAM sequence of NNGG, and binds to a guide RNA comprising: a CRISPR repeat set forth as SEQ ID NO: 16, or an active variant or fragment thereof; and a tracrRNA set forth as SEQ ID NO: 17, or an active variant or fragment thereof.

Non-limiting examples of RGNs useful in the presently disclosed methods and compositions include APG07433.1, APG05586, APG01604, and LPG10145 RNA-guided nucleases, the amino acid sequences of which are set forth, respectively, as SEQ ID NOs: 3, 7, 11, and 15, and active fragments or variants thereof that retain the ability to bind to a target sequence in an RNA-guided sequence-specific manner. In some embodiments, an active variant of an RGN disclosed herein comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NO: 3. In some embodiments, an active variant of an RGN disclosed herein comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NO: 7. In some embodiments, an active variant of an RGN disclosed herein comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NO: 11. In some embodiments, an active variant of an RGN disclosed herein comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NO: 15. In some embodiments, an active fragment of the APG07433.1 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NO: 3. In some embodiments, an active fragment of the APG05586 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NO: 7. In some embodiments, an active fragment of the APG01604 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NO: 11. In some embodiments, an active fragment of the LPG10145 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NO: 15.

The presently disclosed compositions and methods can comprise an RGN capable of binding a target sequence of the disclosure or an RGN having an amino acid sequence set forth as SEQ ID NO: 3, or an active variant or fragment thereof, wherein the RGN is capable of binding a target sequence adjacent to a PAM consensus sequence set forth as NNNNCC. In some embodiments, the PAM sequence is 3′ of the target sequence on its non-target strand. In some embodiments, the RGN binds to a guide RNA having a sequence set forth as SEQ ID NO: 27 or 28. In some embodiments, the RGN binds to a guide RNA comprising a CRISPR repeat set forth as SEQ ID NO: 4, or an active variant or fragment thereof, and a tracrRNA set forth as SEQ ID NO: 5, or an active variant or fragment thereof.

The presently disclosed compositions and methods can comprise an RGN capable of binding a target sequence of the disclosure or an RGN having an amino acid sequence set forth as SEQ ID NO: 7, or an active variant or fragment thereof, wherein the RGN is capable of binding a target sequence adjacent to a PAM consensus sequence set forth as NNRYA. In some embodiments, the PAM sequence is 3′ of the target sequence on its non-target strand. In some embodiments, the RGN binds to a guide RNA having a sequence set forth as SEQ ID NO: 25 or 26. In some embodiments, the RGN binds to a guide RNA comprising a CRISPR repeat set forth as SEQ ID NO: 8 or 106, or an active variant or fragment thereof, and a tracrRNA set forth as SEQ ID NO: 9 or 107, or an active variant or fragment thereof.

The presently disclosed compositions and methods can comprise an RGN capable of binding a target sequence of the disclosure or an RGN having an amino acid sequence set forth as SEQ ID NO: 11, or an active variant or fragment thereof, wherein the RGN is capable of binding a target sequence adjacent to a PAM consensus sequence set forth as NNGRR. In some embodiments, the PAM sequence is 3′ of the target sequence on its non-target strand. In some embodiments, the RGN binds to a guide RNA having a sequence set forth as SEQ ID NO: 29. In some embodiments, the RGN binds to a guide RNA comprising a CRISPR repeat set forth as SEQ ID NO: 12, or an active variant or fragment thereof, and a tracrRNA set forth as SEQ ID NO: 13 or 120, or an active variant or fragment thereof.

The presently disclosed compositions and methods can comprise an RGN capable of binding a target sequence of the disclosure or an RGN having an amino acid sequence set forth as SEQ ID NO: 15, or an active variant or fragment thereof, wherein the RGN is capable of binding a target sequence adjacent to a PAM consensus sequence set forth as NNGG. In some embodiments, the PAM sequence is 3′ of the target sequence on its non-target strand. In some embodiments, the RGN binds to a guide RNA having a sequence set forth as SEQ ID NO: 18. In some embodiments, the RGN binds to a guide RNA comprising a CRISPR repeat set forth as SEQ ID NO: 16, or an active variant or fragment thereof, and a tracrRNA set forth as SEQ ID NO: 17, or an active variant or fragment thereof.

In some embodiments, nucleases other than RGNs are used in the presently disclosed compositions and methods. These nucleases bind to the opposite end of or within the expanded trinucleotide repeat of the HTT gene from the presently disclosed target sequences. As used herein, the term “nuclease” refers to an enzyme that catalyzes the cleavage of phosphodiester bonds between nucleotides in a nucleic acid molecule. In general, the nuclease is an endonuclease, which is capable of cleaving phosphodiester bonds between nucleotides within a nucleic acid molecule. In some embodiments, the sequence-specific nuclease is selected from the group consisting of a meganuclease, a zinc finger nuclease, a TAL-effector DNA binding domain-nuclease fusion protein (TALEN), and an RNA-guided nuclease (RGN) or variants thereof wherein the nuclease activity has been reduced or inhibited.

As used herein, the term “meganuclease” or “homing endonuclease” refers to endonucleases that bind a recognition site within double-stranded DNA that is 12 to 40 bp in length. Non-limiting examples of meganucleases are those that belong to the LAGLIDADG family that comprise the conserved amino acid motif LAGLIDADG (SEQ ID NO: 139). The term “meganuclease” can refer to a dimeric or single-chain meganuclease.

As used herein, the term “zinc finger nuclease” or “ZFN” refers to a chimeric protein comprising a zinc finger DNA-binding domain and a nuclease domain.

As used herein, the term “TAL-effector DNA binding domain-nuclease fusion protein” or “TALEN” refers to a chimeric protein comprising a TAL effector DNA-binding domain and a nuclease domain.

According to the present invention, the presently disclosed target sequences within a mutant HTT allele are bound by an RGN. The target strand of the target sequence hybridizes with the guide RNA associated with the RGN. The target strand and/or the non-target strand of the target sequence (e.g., target DNA sequence) can then be subsequently cleaved by the RGN if the polypeptide possesses nuclease activity. The terms “cleave” or “cleavage” refer to the hydrolysis of at least one phosphodiester bond within the backbone of one or both strands of a double-stranded target sequence (e.g., target DNA sequence) that can result in either single-stranded or double-stranded breaks within the target DNA sequence. The cleavage of a presently disclosed target sequence can result in staggered breaks or blunt ends.

The presently disclosed compositions and methods can utilize RGNs or other nucleases comprising at least one nuclear localization signal (NLS) to enhance transport of the RGN to the nucleus of a cell. Nuclear localization signals are known in the art and generally comprise a stretch of basic amino acids (see, e.g., Lange et al., J. Biol. Chem. (2007) 282:5101-5105). In some embodiments, the RGN comprises 2, 3, 4, 5, 6 or more nuclear localization signals. The nuclear localization signal(s) can be a heterologous NLS. Non-limiting examples of nuclear localization signals useful for the presently disclosed RGNs are the nuclear localization signals of SV40 Large T-antigen, nucleoplasmin, and c-Myc (see, e.g., Ray et al. (2015) Bioconjug Chem 26(6):1004-7). In embodiments, the RGN comprises the NLS sequence set forth as SEQ ID NO: 86, 87, or 125. The RGN or other nuclease can comprise one or more NLS sequences at its N-terminus, C-terminus, or both the N-terminus and C-terminus. For example, the RGN can comprise two NLS sequences at the N-terminal region and four NLS sequences at the C-terminal region. In some embodiments, the RGN or other nuclease comprises a SV40 NLS (such as the sequence set forth as SEQ ID NO: 86) at the N-terminus and a nucleoplasmin NLS (such as the sequence set forth as SEQ ID NO: 87) at its C-terminus. In some embodiments, the RGN or other nuclease comprises a c-Myc NLS (such as the sequence set forth as SEQ ID NO: 125) at both its N-terminus and its C-terminus. When an NLS is attached at the N-terminus, C-terminus, or both, of an RGN or other nuclease, an NLS linker protein can be present to separate the RGN or other nuclease from the NLS. In some embodiments, an NLS linker protein connects an RGN polypeptide or other nuclease to an NLS. Such an NLS linker protein can be 1, 2, 3, 4, 5, 6, 7, 8, 9, 10, 11, 12, 13, 14, 15, or more amino acids in length. In some embodiments, the NLS linker protein is at least 1, at least 2, at least 3, at least 4, at least 5, at least 6, at least 7, or at least 8 amino acids in length. In some embodiments, the NLS linker protein between or connecting an NLS and an RGN or other nuclease has the sequence set forth as SEQ ID NO: 127. In some embodiments, the RGN or other nuclease comprises a c-Myc NLS (such as the sequence set forth as SEQ ID NO: 125) at its N-terminus, separated from the nuclease protein by an NLS linker protein having the sequence set forth as SEQ ID NO: 127, and a c-Myc NLS (such as the sequence set forth as SEQ ID NO: 125) at its C-terminus, separated from the nuclease protein by an NLS linker protein having the sequence set forth as SEQ ID NO: 127. The RGN polypeptide or other nuclease can be connected to a c-Myc NLS (such as the sequence set forth as SEQ ID NO: 125) at the N-terminus and to a c-Myc NLS (such as the sequence set forth as SEQ ID NO: 125) at the C-terminus of the RGN polypeptide or other nuclease, wherein the RGN polypeptide or other nuclease is connected to each of the N-terminal c-Myc NLS and C-terminal c-Myc NLS by an NLS linker protein having the sequence set forth as SEQ ID NO: 127.

In some embodiments, the presently disclosed compositions and methods utilize RGNs or other nucleases comprising at least one cell-penetrating domain that facilitates cellular uptake of the RGN. Cell-penetrating domains are known in the art and generally comprise stretches of positively charged amino acid residues (i.e., polycationic cell-penetrating domains), alternating polar amino acid residues and non-polar amino acid residues (i.e., amphipathic cell-penetrating domains), or hydrophobic amino acid residues (i.e., hydrophobic cell-penetrating domains) (see, e.g., Milletti F. (2012) Drug Discov Today 17:850-860). A non-limiting example of a cell-penetrating domain is the trans-activating transcriptional activator (TAT) from the human immunodeficiency virus 1.

The nuclear localization signal and/or cell-penetrating domain can be located at the N-terminus, the C-terminus, and/or in an internal location of the RGN.

V. Nucleic Acid Molecules Encoding RNA-Guided Nucleases, Single Guide RNAs, CRISPR RNAs, and or tracrRNAs

The present disclosure provides nucleic acid molecules comprising or encoding the presently disclosed RGNs, crRNAs, tracrRNAs, and/or sgRNAs.

The use of the term “polynucleotide” or “nucleic acid molecule” is not intended to limit the present disclosure to polynucleotides comprising DNA. Those of ordinary skill in the art will recognize that polynucleotides can comprise ribonucleotides (RNA) and combinations of ribonucleotides and deoxyribonucleotides. Such deoxyribonucleotides and ribonucleotides include both naturally occurring molecules and synthetic analogues. These include peptide nucleic acids (PNAs), PNA-DNA chimers, locked nucleic acids (LNAs), and phosphothiorate linked sequences. The polynucleotides disclosed herein also encompass all forms of sequences including, but not limited to, single-stranded forms, double-stranded forms, DNA-RNA hybrids, triplex structures, stem-and-loop structures, and the like.

In some of those embodiments wherein the presently disclosed compositions and methods comprise a nucleic acid molecule encoding an RGN, the nucleic acid molecule is an mRNA (messenger RNA) molecule. An mRNA refers to any polynucleotide which encodes a polypeptide of interest and which is capable of being translated to produce the encoded polypeptide of interest in vitro, in vivo, in situ, or ex vivo. In some embodiments, the basic components of an mRNA molecule include at least a coding region, a 5′UTR, a 3′UTR, a 5′ cap and a poly-A tail. In some embodiments, an mRNA encoding an RGN useful in the presently disclosed methods and compositions can include one or more structural and/or chemical modifications or alterations which impart useful properties to the polynucleotide. For instance, a useful property of an mRNA includes the lack of a substantial induction of the innate immune response of a cell into which the mRNA is introduced. A “structural” feature or modification is one in which two or more linked nucleotides are inserted, deleted, duplicated, inverted or randomized in an mRNA without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. Chemical modifications to mRNA can involve inclusion of 5-methylcytosine, N1-methyl-pseudouridine, pseudouridine, 2-thiouridine, 4-thiouridine, 5-methoxyuridine, 2′Fluoroguanosine, 2′Fluorouridine, 5-bromouridine, 5-(2-carbomethoxyvinyl) uridine, 5-[3(1-E-propenylamino)]uridine, α-thiocytidine, N6-methyladenosine, 5-methylcytidine, N4-acetylcytidine, 5-formylcytidine, or combinations thereof, in an mRNA.

The nucleic acid molecules encoding RGNs can be codon optimized for expression in an organism of interest (e.g., mammal). A “codon-optimized” coding sequence is a polynucleotide coding sequence having its frequency of codon usage designed to mimic the frequency of preferred codon usage or transcription conditions of a particular host cell. Expression in the particular host cell or organism is enhanced as a result of the alteration of one or more codons at the nucleic acid level such that the translated amino acid sequence is not changed. Nucleic acid molecules can be codon optimized, either wholly or in part. Codon tables and other references providing preference information for a wide range of organisms are available in the art (see, e.g., Gaspar et al. (2012) Bioinformatics 28(20): 2683-2684; Komar et al. (1998) Biol. Chem. 379(10): 1295-1300; and Inouye et al. (2015) Protein Expr. Purif 109: 47-54). A non-limiting example of a codon-optimized coding sequence for an RGN useful in the presently disclosed compositions and methods is set forth as SEQ ID NO: 88.

Polynucleotides encoding the RGNs, crRNAs, tracrRNAs, and/or sgRNAs provided herein can be provided in expression cassettes for in vitro expression or expression in a cell, embryo, or organism of interest. The cassette will include 5′ and 3′ regulatory sequences operably linked to a polynucleotide encoding an RGN, a crRNA, a tracrRNA, and/or an sgRNA provided herein that allows for expression of the polynucleotide. The cassette may additionally contain at least one additional gene or genetic element to be co-transformed into the organism. Where additional genes or elements are included, the components are operably linked. The term “operably linked” is intended to mean a functional linkage between two or more elements. For example, an operable linkage between a promoter and a coding region of interest (e.g., region coding for an RGN, a crRNA, a tracrRNA, and/or an sgRNA) is a functional link that allows for expression of the coding region of interest. Operably linked elements may be contiguous or non-contiguous. When used to refer to the joining of two protein coding regions, by “operably linked” or “operably fused” is intended that the coding regions are in the same reading frame. For example, polypeptides that are “operably fused” can mean that the structure and/or biological activity of each individual peptide is also present in the fusion. Alternatively, the additional gene(s) or element(s) can be provided on multiple expression cassettes. For example, the nucleotide sequence encoding a presently disclosed RGN can be present on one expression cassette, whereas the nucleotide sequence encoding a crRNA, a tracrRNA, or a complete guide RNA can be on a separate expression cassette. Such an expression cassette is provided with a plurality of restriction sites and/or recombination sites for insertion of the polynucleotides to be under the transcriptional regulation of the regulatory regions. The expression cassette may additionally contain a selectable marker gene.

The expression cassette will include in the 5′-3′ direction of transcription, a transcriptional (and, in some embodiments, translational) initiation region (i.e., a promoter), an RGN-, crRNA-, tracrRNA- and/or sgRNA-encoding polynucleotide of the disclosure, and a transcriptional (and in some embodiments, translational) termination region (i.e., termination region) functional in the organism of interest. The promoters of the disclosure are capable of directing or driving expression of a coding sequence in a host cell. The regulatory regions (e.g., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention. As used herein, a chimeric gene comprises a coding sequence operably linked to a transcription initiation region that is heterologous to the coding sequence.

Convenient termination regions include ones from simian virus (SV40), human growth hormone (hGH), bovine growth hormone (BGH), and rabbit beta-globin (rbGlob). See also Proudfoot (1991) Cell 64:671-674; Munroe et al. (1990) Gene 91:151-158; Schek et al. (1992) Molecular and Cellular Biology 12(12):5386-5393; Gil and Proudfoot (1987) Cell 49(3):399-406; Goodwin and Rottman (1992) The Journal ofBiological Chemistry 267(23):16330-16334; and Lanoix and Acheson (1988) EMBO J. 7(8): 2515-2522.

Additional regulatory signals include, but are not limited to, transcriptional initiation start sites, operators, activators, enhancers, other regulatory elements, ribosomal binding sites, an initiation codon, termination signals, and the like. See, for example, Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. Generally, expression of the RGN will be under the control of an RNA polymerase II promoter and RGN-coding sequences can thus be operably linked to an RNA polymerase II promoter. Expression of the crRNA, tracrRNA, or sgRNA will generally be under the control of an RNA polymerase III promoter and coding sequences for these elements can thus be operably linked to an RNA polymerase III promoter. Non-limiting examples of RNA polymerase III promoters useful for the expression of crRNAs, tracrRNAs and sgRNAs are the mammalian U6, U3, H1, and 7SL RNA promoters and rice U6 and U3 promoters, such as the human U6 small nuclear promoter or a truncated version thereof, such as the sequences set forth as SEQ ID NO: 89 or 128, as well as the promoters disclosed in U.S. Provisional Appl. No. 63/209,660, filed Jun. 11, 2021, and International Application No. PCT/US2022/032940, filed Jun. 10, 2022, each of which is herein incorporated by reference in its entirety, including promoters set forth herein as SEQ ID NOs: 96-105.

The nucleic acids can be combined with constitutive, inducible, growth stage-specific, cell type-specific, tissue-preferred, tissue-specific, or other promoters for expression in the organism of interest.

Exemplary constitutive promoters for expression in cells of the present disclosure include: an SV40 early promoter; a mouse mammary tumor virus long terminal repeat (LTR) promoter; adenovirus major late promoter (Ad MLP); a herpes simplex virus (HSV) promoter; a cytomegalovirus (CMV) promoter such as the CMV immediate early promoter region (CMVIE); a rous sarcoma virus (RSV) promoter; a human ubiquitin C promoter (UBC); a human U6 small nuclear promoter (U6); a truncated U6 promoter; an enhanced U6 promoter; a human H1 promoter from RNA polymerase III (H1); a human elongation factor 1α promoter (EF1A); a human beta-actin promoter (ACTB); a human or mouse phosphoglycerate kinase 1 promoter (PGK); a chicken β-Actin promoter coupled with CMV early enhancer (CAGG); a yeast transcription elongation factor promoter (TEF1); an elongation factor 1α short (EFS) promoter; a JeT promoter (see, for example, U.S. Publ. No. 2002/0098547, which is incorporated by reference in its entirety); and the like. See, for example, Miyagishi et al. (2002) Nature Biotechnology 20:497-500; Xia et al. (2003) Nucleic Acids Res. 31(17):e100-e100; Pasleau et al. (1985) Gene 38:227-232; Martin-Gallardo et al. (1988) Gene 70: 51-56; Oellig and Seliger (1990) J Neurosci Res 26: 390-396; Manthorpe et al. (1993) Hum Gene Ther 4: 419-431; Yew et al. (1997) Hum Gene Ther 8: 575-584; Xu et al. (2001) Gene 272: 149-156; Nguyen et al. (2008) J Surg Res 148: 60-66; Costa et al. (2005) Nat Meth. 2:259-260; Lam and Truong (2020) ACS Synth. Biol. 9(10):2625-2631. In some embodiments, the RGN-encoding sequence is operably linked to a constitutive promoter, which can be a cytomegalovirus (CMV) promoter, a truncated CMV promoter, such as the CMVeb promoter set forth as SEQ ID NO: 90, an elongation factor 1α short (EFS) promoter set forth as SEQ ID NO: 91, or a JeT promoter set forth as SEQ ID NO: 92.

Examples of inducible promoters include: stress-regulated promoters such as Hsp70 and Hsp90 promoters (Wurm et al. (1986) Proc. Natl. Acad. Sci. USA. 83:5414-5418; Nover L. Heat Shock Response. CRC Press; Boca Raton, FL, USA: 1991); metal-regulated promoters (Mayo et al. (1982) Cell. 29:99-108; Searle et al. (1985) Mol. Cell. Biol. 5:1480-1489); hormone-responsive promoters including a glucocorticoid-responsive promoter (Hynes et al. (1981) Proc. Natl. Acad. Sci. USA. 78:2038-2042; Klock et al. (1987) Nature. 329:734-736). Chemically regulated promoters from prokaryotes that have been used include isopropyl-beta-D-thiogalactopyranoside (IPTG)-regulated promoters, lactose-regulated promoters, and tetracycline-regulated promoters (see, for example, Gossen et al. (1993) Trends Biochem Sci. 18:471-475; Gossen and Bujard (1992) Proc. Natl Acad. Sci. USA 89:5547-5551; Zhou et al. (2006) Gene Ther. 13:1382-1390). Inducible expression can be obtained using operator systems including AlcR/acetaldehyde, ArgR/L-arginine, BirA/biotinyl-AMP, CymR/cumate, EthR/2-phenylethylbutyrate, HdnoR/6-hydroxynicotine, HucR/uric acid, MphR(A)/macrolides, PIP/Streptogramins, Rex/NADH, RheA/heat, ScbR/SCB1, TraR/3-oxo-C8-HSL, and TtgR/phloretin; see, for example, U.S. Pat. No. 8,728,759B2; U.S. Pat. No. 7,745,592B2; Weber and Fussenegger (2004) Methods Mol. Biol. 267:451-466; Hartenbach et al. (2007) Nucleic Acids Res. 35:e136; Weber et al. (2009) Metab. Eng. 11:117-124; Weber et al. (2008) Proc. Natl. Acad. Sci. USA. 105:9994-9998; Malphettes et al. (2005) Nucleic Acids Res. 33:e107; Kemmer et al. (2010) Nat. Biotechnol. 28:355-360; Weber et al. (2002) Nat. Biotechnol. 20:901-907; Fussenegger et al. (2000) Nat. Biotechnol. 18:1203-1208; Weber et al. (2006) Metab. Eng. 8:273-280; Weber et al. (2003) Nucleic Acids Res. 31:e69; Weber et al. (2003) Nucleic Acids Res. 31:e71; Neddermann et al. (2003) EMBO Rep. 4:159-165; and Gitzinger et al. (2009) Proc. Natl. Acad. Sci. USA. 106:10638-10643. Inducible expression can be obtained using protein-protein interaction systems including: rapamycin-induced interaction between FKBP12 (FK506 binding protein 12) and mTOR (Rivera et al. (1996) Nat. Med. 2:1028-1032; Belshaw et al. (1996) Proc. Natl. Acad. Sci. USA. 93:4604-46077); abscisic acid (ABA)-regulated interaction between PYL1 (abscisic acid receptor) and ABI1 (protein phosphatase 2C56) (Liang et al. (2011) Sci. Signal. 4(164):rs2-rs2); and light-induced protein-protein interaction systems (Wang et al. (2012) Nat. Methods. 9:266-269; Yamada et al. (2018) Cell. Rep. 25:487-500).

Tissue-specific or tissue-preferred promoters can be utilized to target expression of an expression construct within a particular tissue. In embodiments, the tissue-specific or tissue-preferred promoters are active in mammalian tissue. Examples of tissue-specific or tissue-preferred promoters include promoters that initiate transcription preferentially in certain tissues, such as the brain. A “tissue specific” promoter is a promoter that initiates transcription only in certain tissues. Unlike constitutive expression of genes, tissue-specific expression is the result of several interacting levels of gene regulation. As such, promoters from homologous or closely related species can be preferable to use to achieve efficient and reliable expression of transgenes in particular tissues. In some embodiments, the expression comprises a tissue-preferred promoter. A “tissue preferred” promoter is a promoter that initiates transcription preferentially, but not necessarily entirely or solely in certain tissues, such as the brain.

In embodiments, the nucleic acid molecules encoding an RGN, crRNA, tracrRNA, and/or sgRNA comprise a cell type-specific promoter. A “cell type specific” promoter is a promoter that primarily drives expression in certain cell types in one or more organs. Some examples of cells in which cell type specific promoters may be primarily active include, for example, a neuron. The nucleic acid molecules can also include cell type preferred promoters. A “cell type preferred” promoter is a promoter that primarily drives expression mostly, but not necessarily entirely or solely in certain cell types in one or more organs. Some examples of cells in which cell type preferred promoters may be preferentially active include, for example, a neuron. Neurons can include neural progenitor cells, forebrain neuron progenitor cells, striatal neurons, medium spiny neurons, and cortical neurons. Cell type preferred promoters may be preferentially active in the brain in a non-neuronal cell, such as a glial cell. Glial cells can include microglia, astrocytes, and oligodendrocytes. In some embodiments, cell type preferred promoters may be preferentially active in putamen, caudate, striatum, cerebral cortex, globus pallidus, hippocampus, amygdala, thalamus, hypothalamus, subthalamic nucleus, substantia nigra, cerebellum, brainstem, or a combination thereof, of the brain.

An RGN-encoding sequence can be operably linked to a brain or neuron-specific promoter, such as the human synapsin I (Syn) promoter, the 65 kDa or 67 kDa glutamic acid decarboxylase (GAD65 or GAD67, respectively) promoter, the homeobox Dlx5/6 promoter, the preprotachykinin 1 (Tac1) promoter, the neuron-specific enolase (NSE), the dopaminergic receptor 1 (Drd1a) promoter or dopaminergic receptor 2 (DRD2) promoter, glial fibrillary acidic protein (GFAP) promoter, or the 32 kDa dopamine and cyclic AMP-regulated phosphoprotein (DARP32) promoter (see, for example, Delzor et al., 2012, Hum Gene Ther Methods 23(4):242-254, which is incorporated by reference in its entirety). A non-limiting example of a promoter that can be used to drive the expression of an RGN for use in the presently disclosed compositions and methods that is neuron-specific is the human synapsin I (Syn) promoter. The Syn promoter can have the nucleotide sequence set forth as SEQ ID NO: 93.

The nucleic acid sequences encoding the RGNs, crRNAs, tracrRNAs, and/or sgRNAs can be operably linked to a promoter sequence that is recognized by a phage RNA polymerase for example, for in vitro mRNA synthesis. In embodiments, the in vitro-transcribed RNA can be purified for use in the methods described herein. For example, the promoter sequence can be a T7, T3, or SP6 promoter sequence or a variation of a T7, T3, or SP6 promoter sequence. In embodiments, the expressed protein and/or RNAs can be purified for use in the methods of genome modification described herein.

In embodiments, the polynucleotide encoding the RGN, crRNA, tracrRNA, and/or sgRNA also can be linked to a polyadenylation (polyA) signal and/or at least one transcriptional termination sequence. In some embodiments, a coding sequence (e.g., nucleic acid molecule encoding the RGN, crRNA, tracrRNA, and/or sgRNA) is linked to a simian virus (SV40) polyA tail such as the one set forth as SEQ ID NO: 94, or a bovine growth hormone polyadenylation (bGHpolyA) tail such as the one set forth as SEQ ID NO: 95. See, for example, Proudfoot (1991) Cell 64:671-674; Munroe et al. (1990) Gene 91:151-158; Schek et al. (1992) Molecular and Cellular Biology 12(12):5386-5393; Gil and Proudfoot (1987) Cell 49(3):399-406; Goodwin and Rottman (1992) The Journal ofBiological Chemistry 267(23):16330-16334; and Lanoix and Acheson (1988) EMBO J. 7(8): 2515-2522.

Additionally, the sequence encoding the RGN also can be linked to sequence(s) encoding at least one nuclear localization signal, at least one cell-penetrating domain, and/or at least one signal peptide capable of trafficking proteins to particular subcellular locations, as described elsewhere herein.

The polynucleotide encoding the RGN, crRNA, tracrRNA, and/or sgRNA can be present in a vector or multiple vectors. A “vector” refers to a polynucleotide composition for transferring, delivering, or introducing a nucleic acid into a host cell. Suitable vectors include plasmid vectors, phagemids, cosmids, artificial/mini-chromosomes, transposons, and viral vectors (e.g., lentiviral vectors, adeno-associated viral vectors, baculoviral vector). The vector can comprise additional expression control sequences (e.g., enhancer sequences, Kozak sequences, polyadenylation sequences, transcriptional termination sequences), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001.

The vector can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT). Marker genes can include genes that allow selection for growth on a particular nutrient or substance, such as dihydrofolate reductase (DHFR; Simonsen and Levinson (1983) Proc. Natl. Acad. Sci. U.S.A. 80:2495-2499), histidinol dehydrogenase (hisD; Hartman and Mulligan (1988) Proc. Natl. Acad. Sci. U.S.A. 85:8047-8051), puromycin-N-acetyl transferase (PAC or puro; de la Luna et al. (1988) Gene 62:121-126), thymine kinase (TK; Littlefield (1964) Science 145:709-710), and xanthine-guanine phosphoribosyltransferase (XGPRT or gpt; Mulligan and Berg (1981) Proc. Natl. Acad. Sci. U.S.A. 78:2072-2076).

As indicated, expression constructs comprising nucleotide sequences encoding an RGN, a crRNA, a tracrRNA, and/or an sgRNA can be used to transform organisms of interest. Methods for transformation involve introducing a nucleotide construct into an organism of interest. By “introducing” is intended to introduce the nucleotide construct to the host cell in such a manner that the construct gains access to the interior of the host cell. The methods of the disclosure do not require a particular method for introducing a nucleotide construct to a host organism, only that the nucleotide construct gains access to the interior of at least one cell of the host organism. The host cell can be a eukaryotic or prokaryotic cell. In some embodiments, the eukaryotic host cell is a mammalian cell, an avian cell, or an insect cell. In some embodiments, the eukaryotic cell that comprises or expresses a presently disclosed crRNA, tracrRNA, sgRNA, and/or RGN or that has been modified by a presently disclosed RGN system is a human cell. In some embodiments, the eukaryotic cell that comprises or expresses a presently disclosed crRNA, tracrRNA, sgRNA, and/or RGN or that has been modified by a presently disclosed RGN system is a stem cell, including an induced pluripotent stem cell. In some embodiments, the mammalian or human cell that comprises or expresses a presently disclosed crRNA, tracrRNA, sgRNA, and/or RGN or that has been modified by a presently disclosed RGN system is a cardiomyocyte, a neuronal cell, a glial cell, or a retinal ganglia cell.

Methods for introducing nucleotide constructs into host cells are known in the art including, but not limited to, stable transformation methods, transient transformation methods, and virus-mediated methods.

The presently disclosed methods can result in a transformed organism or cell line derived from these transformed cells.

“Transgenic organisms” or “transformed organisms” or “stably transformed” organisms or cells or tissues refers to organisms that have incorporated or integrated a polynucleotide encoding an RGN, a crRNA, a tracrRNA, and/or an sgRNA of the disclosure. It is recognized that other exogenous or endogenous nucleic acid sequences or DNA fragments may also be incorporated into the host cell. Transformation of a host cell may be performed by infection, conjugation, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, and viral mediated, liposome mediated and the like. Viral-mediated introduction of a polynucleotide encoding an RGN, a crRNA, a tracrRNA, and/or an sgRNA includes retroviral, lentiviral, adenoviral, and adeno-associated viral mediated introduction and expression.

Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. “Transient transformation” is intended to mean that a polynucleotide is introduced into the host cell and does not integrate into the genome of the host cell.

In some embodiments, cells that have been transformed may be introduced into an organism. These cells could have originated from the organism, wherein the cells are transformed in an ex vivo approach. These cells can be autologous (originated and returned to the same subject), allogeneic (the donor and recipient subjects are of the same species). In general, the donor and recipient of allogeneic cells are a complete or partial HLA match.

The polynucleotides encoding the RGNs, crRNAs, tracrRNAs, and/or sgRNAs or comprising the crRNAs, tracrRNAs, and/or sgRNAs can also be used to transform any prokaryotic species, including but not limited to, archaea and bacteria (e.g., Bacillus sp., Klebsiella sp. Streptomyces sp., Rhizobium sp., Escherichia sp., Pseudomonas sp., Salmonella sp., Shigella sp., Vibrio sp., Yersinia sp., Mycoplasma sp., Agrobacterium, Lactobacillus sp.).

The polynucleotides encoding the RGNs, crRNAs, tracrRNAs, and/or sgRNAs or comprising the crRNAs, tracrRNAs, and/or sgRNAs can be used to transform any eukaryotic species, including but not limited to animals (e.g., mammals, humans, mice, rats, non-human primates, insects, fish, birds, and reptiles), fungi, amoeba, algae, and yeast.

Conventional viral and non-viral based gene transfer methods can be used to introduce nucleic acids in mammalian, insect, or avian cells or target tissues. Such methods can be used to administer nucleic acids encoding components of an RGN system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256: 808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Viral. 66:2731-2739 (1992); Johann et al., J. Viral. 66:1635-1640 (1992); Sommnerfelt et al., Viral. 176:58-59 (1990); Wilson et al., J. Viral. 63:2374-2378 (1989); Miller et al., J. Viral. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Katin, Human Gene Therapy 5:793-801 (1994); Muzyczka, 1. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Viral. 63:03822-3828 (1989). Packaging cells are typically used to form virus particles that are capable of infecting a host cell. Such cells include 293 cells, which package adenovirus, and ψJ2 cells or PA317 cells, which package retrovirus.

Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences.

The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

Non-limiting examples of AAV vectors useful in the presently disclosed compositions and methods are AAV2, AAV3, AAV5, AAV6, and AAV9 vectors (see, for example, Pupo et al., 2022, Molecular Therapy 30(12):P3515-3541, which is incorporated by reference in its entirety). In some embodiments, the vector is AAV5 or AAV6. In some embodiments, the AAV vector has the sequence set forth as any one of SEQ ID NOs: 30-39 or 121-123 and the AAV has packaged the vector sequences.

In some embodiments, a host cell is transiently or non-transiently transfected with one or more nucleic acid molecules or vectors described herein. In some embodiments, a cell is transfected as it naturally occurs in a subject. In some embodiments, a cell that is transfected is taken from a subject, such as a Huntington's disease patient. In embodiments, the cell is derived from cells taken from a subject, such as a cell line. In some embodiments, the cell line may be mammalian, insect, or avian cells. A wide variety of cell lines for tissue culture are known in the art. Examples of cell lines include, but are not limited to, C8161, CCRF-CEM, MOLT, mIMCD-3, NHDF, HeLaS3, Huh1, Huh4, Huh7, HUVEC, HASMC, HEKn, HEKa, MiaPaCell, Panel, PC-3, TF1, CTLL-2, CIR, Rat6, CVI, RPTE, AlO, T24, 182, A375, ARH-77, Calu1, SW480, SW620, SKOV3, SK-UT, CaCo2, P388D1, SEM-K2, WEHI−231, HB56, TIB55, lurkat, 145.01, LRMB, Bcl-1, BC-3, IC21, DLD2, Raw264.7, NRK, NRK-52E, MRC5, MEF, Hep G2, HeLa B, HeLa T4. COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-I cells, BEAS-2B, bEnd.3, BHK-21, BR 293, BxPC3, C3H-10T1/2, C6/36, Cal-27, CHO, CHO-7, CHO-1R, CHO-K1, CHO-K2, CHO-T, CHO Dhfr-/-, COR-L23, COR-L23/CPR, COR-L235010, CORL23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, lurkat, IY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCKII, MDCKII, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, a cell transfected with one or more nucleic acid molecules or vectors described herein is used to establish a new cell line comprising one or more vector-derived sequences. In some embodiments, a cell transiently transfected with the components of an RGN system as described herein (such as by transient transfection of one or more vectors, or transfection with RNA), and modified through the activity of an RGN system, is used to establish a new cell line comprising cells containing the modification but lacking any other exogenous sequence.

In some embodiments, one or more nucleic acid molecules or vectors described herein are used to produce a non-human transgenic animal. In some embodiments, the transgenic animal is a mammal, such as a mouse, rat, hamster, rabbit, cow, or pig.

### VI. Variants and Fragments of Polypeptides and Polynucleotides

The present disclosure provides active variants and fragments of the presently disclosed crRNA repeats, crRNAs, tracrRNAs, sgRNAs, and RGNs. An active variant or fragment of a naturally-occurring (i.e., wild-type) RGN binds to a target sequence described herein within a mutant HTT allele in an RNA-guided sequence-specific manner. In some embodiments, a target sequence described herein includes the nucleotide sequence set forth as any one of SEQ ID NOs: 75-79, and 130. In some embodiments, the disclosure provides active variants and fragments of an RGN having an amino acid sequence set forth as any one of SEQ ID NOs: 3, 7, 11, and 15, as well as active variants and fragments of naturally-occurring CRISPR repeats, including sequences set forth as any one of SEQ ID NOs: 4, 8, 12, 16, and 106, active variants and fragments of naturally-occurring tracrRNAs, such as any one of the sequences set forth as any one of SEQ ID NOs: 5, 9, 13, 17, 107, and 120, and active variants and fragments of sgRNAs, such as sequences set forth as any one of SEQ ID NOs: 25-29, and polynucleotides encoding the same. In some embodiments, sgRNAs of the disclosure include an sgRNA set forth as SEQ ID NO: 6, 10, 14, or 18, wherein the sgRNA comprises any spacer useful for targeting a target sequence in a mHTT allele and a backbone that can be bound by an RGN polypeptide of SEQ ID NO: 3, 7, 11, or 15, respectively, or an active variant or fragment thereof.

While the activity of a variant or fragment may be altered compared to the polynucleotide or polypeptide of interest, the variant and fragment should retain the functionality of the polynucleotide or polypeptide of interest. For example, a variant or fragment may have increased activity, decreased activity, different spectrum of activity or any other alteration in activity when compared to the polynucleotide or polypeptide of interest.

Fragments and variants of naturally-occurring RGN polypeptides, such as those disclosed herein, will retain sequence-specific, RNA-guided DNA-binding activity. In embodiments, fragments and variants of naturally-occurring RGN polypeptides, such as those disclosed herein, retain nuclease activity (single-stranded or double-stranded).

Fragments and variants of naturally-occurring CRISPR repeats, such as those disclosed herein, will retain the ability, when part of a guide RNA (comprising a tracrRNA), to bind to and guide an RNA-guided nuclease (complexed with the guide RNA) to a target sequence in a sequence-specific manner.

Fragments and variants of naturally-occurring tracrRNAs, such as those disclosed herein, will retain the ability, when part of a guide RNA (comprising a CRISPR RNA), to guide an RNA-guided nuclease (complexed with the guide RNA) to a target sequence in a sequence-specific manner.

Fragments and variants of sgRNAs, such as those disclosed herein, will retain the ability to guide an RNA-guided nuclease (complexed with the sgRNA) to a target sequence in a sequence-specific manner.

The term “fragment” refers to a portion of a polynucleotide or polypeptide sequence of the disclosure. “Fragments” or “biologically active portions” include polynucleotides comprising a sufficient number of contiguous nucleotides to retain the biological activity (i.e., binding to and directing an RGN in a sequence-specific manner to a target sequence when comprised within a guide RNA). “Fragments” or “biologically active portions” include polypeptides comprising a sufficient number of contiguous amino acid residues to retain the biological activity (i.e., binding to a target sequence in a sequence-specific manner when complexed with a guide RNA). Fragments of the RGN proteins include those that are shorter than the full-length sequences due to the use of an alternate downstream start site. A biologically active portion of an RGN protein can be a polypeptide that comprises, for example, 10, 25, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700 or more contiguous amino acid residues of an RGN that binds a target nucleotide sequence disclosed herein or of an RGN having the amino acid sequence set forth as any one of SEQ ID NOs: 3, 7, 11, and 15. Such biologically active portions can be prepared by recombinant techniques and evaluated for sequence-specific, RNA-guided DNA-binding activity. A biologically active fragment of a CRISPR repeat sequence can comprise at least 8 contiguous nucleotides of any one of SEQ ID NOs: 4, 8, 12, 16, and 106. A biologically active portion of a CRISPR repeat sequence can be a polynucleotide that comprises, for example, 8, 9, 10, 11, 12, 13, 14, 15, 16, 17, 18, 19, or 20 contiguous nucleotides of any one of SEQ ID NOs: 4, 8, 12, 16, and 106. A biologically active portion of a tracrRNA can be a polynucleotide that comprises, for example, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80 or more contiguous nucleotides of any one of SEQ ID NOs: 5, 9, 13, 17, 107, and 120. A biologically active portion of a sgRNA can be a polynucleotide that comprises, for example, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more contiguous nucleotides of any one of SEQ ID NOs: 6, 10, 14, 18, and 25-29.

In general, “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” or “wild type” polynucleotide or polypeptide comprises a naturally occurring nucleotide sequence or amino acid sequence, respectively. For polynucleotides, conservative variants include those sequences that, because of the degeneracy of the genetic code, encode the native amino acid sequence of the gene of interest. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the polypeptide or the polynucleotide of interest. Generally, variants of a particular polynucleotide disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide disclosed herein (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides disclosed herein is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

In certain embodiments, the presently disclosed polynucleotides encode an RNA-guided nuclease polypeptide comprising an amino acid sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to an amino acid sequence encoding an RGN that binds a target sequence disclosed herein or an amino acid sequence set forth as any one of SEQ ID NOs: 75-79, and 130.

A biologically active variant of an RGN polypeptide of the disclosure may differ by as few as about 1-15 amino acid residues, as few as about 1-10, such as about 6-10, as few as 5, as few as 4, as few as 3, as few as 2, or as few as 1 amino acid residue. In some embodiments, the polypeptides can comprise an N-terminal or a C-terminal truncation, which can comprise at least a deletion of 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700 amino acids or more from either the N or C terminus of the polypeptide.

In some embodiments, the presently disclosed polynucleotides comprise or encode a crRNA repeat comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as any one of SEQ ID NOs: 4, 8, 12, 16, and 106.

The presently disclosed polynucleotides can comprise or encode a tracrRNA comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to any one of the nucleotide sequences set forth as any one of SEQ ID NOs: 5, 9, 13, 17, 107, and 120.

The presently disclosed polynucleotides can comprise or encode an sgRNA comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to any one of the nucleotide sequences set forth as SEQ ID NOs: 6, 10, 14, 18, and 25-29.

Biologically active variants of a CRISPR repeat, crRNA, tracrRNA, or sgRNA of the disclosure may differ by as few as about 1-15 nucleotides, as few as about 1-10, such as about 6-10, as few as 5, as few as 4, as few as 3, as few as 2, or as few as 1 nucleotide. In some embodiments, the polynucleotides can comprise a 5′ or 3′ truncation, which can comprise at least a deletion of 10, 15, 20, 25, 30, 35, 40, 45, 50, 55, 60, 65, 70, 75, 80, 90, 95, 100, 105, 110 nucleotides or more from either the 5′ or 3′ end of the polynucleotide.

As used herein, a sequence that “differs from” a parental sequence by a certain number of amino acids or nucleotides can differ due to amino acid or nucleotide substitutions, amino acid or nucleotide additions, and/or amino acid or nucleotide deletions. For example, a nucleotide sequence that differs from a parental nucleotide sequence by 1 nucleotide can have a single nucleotide substitution, can be one nucleotide longer than the parental sequence, or can be one nucleotide shorter than the parental sequence.

It is recognized that modifications may be made to the RGN polypeptides, CRISPR repeats, crRNAs, tracrRNAs, and sgRNAs provided herein creating variant proteins and polynucleotides. Changes designed by man may be introduced through the application of site-directed mutagenesis techniques.

Alternatively, native, as yet-unknown, or as yet unidentified polynucleotides and/or polypeptides structurally and/or functionally-related to the sequences disclosed herein may also be identified that fall within the scope of the present disclosure. Conservative amino acid substitutions may be made in non-conserved regions that do not alter the function of the RGN proteins. Alternatively, modifications may be made that improve the activity of the RGN.

Variant polynucleotides and proteins also encompass sequences and proteins derived from a mutagenic and recombinogenic procedure such as DNA shuffling. With such a procedure, one or more different RGN proteins disclosed herein (e.g., SEQ ID NOs: 3, 7, 11, or 15) is manipulated to create a new RGN protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the RGN sequences provided herein and other known RGN genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458. A “shuffled” nucleic acid is a nucleic acid produced by a shuffling procedure such as any shuffling procedure set forth herein. Shuffled nucleic acids are produced by recombining (physically or virtually) two or more nucleic acids (or character strings), for example in an artificial, and optionally recursive, fashion. Generally, one or more screening steps are used in shuffling processes to identify nucleic acids of interest; this screening step can be performed before or after any recombination step. In some (but not all) shuffling embodiments, it is desirable to perform multiple rounds of recombination prior to selection to increase the diversity of the pool to be screened. The overall process of recombination and selection are optionally repeated recursively. Depending on context, shuffling can refer to an overall process of recombination and selection, or, alternately, can simply refer to the recombinational portions of the overall process.

As used herein, “sequence identity” or “identity” in the context of two polynucleotides or polypeptide sequences makes reference to the residues in the two sequences that are the same when aligned for maximum correspondence over a specified comparison window. It is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Protein sequences that differ by such conservative substitutions are said to have “sequence similarity” or “similarity”. Means for measuring sequence similarity are well known to those of skill in the art. Typically, this involves scoring a conservative substitution as a partial rather than a full mismatch. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. O. Dayhoff), pp. 345-352. Natl. Biomed. Res. Found., Washington, D.C. and Henikoff et al. (1992) Proc. Natl. Acad. Sci. USA 89:10915-10919. The BLOSUM62 matrix is often used as a default scoring substitution matrix in sequence alignment protocols. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al. (1997) Nucleic Acids Res. 25:3389-3402, and made available to the public at the National Center for Biotechnology Information Website (www.ncbi.nlm.nih.gov). Optimal alignments, including multiple alignments, can be prepared using, e.g., PSI-BLAST, available through www.ncbi.nlm.nih.gov and described by Altschul et al. (1997) Nucleic Acids Res. 25:3389-3402.

With respect to a nucleotide sequence or an amino acid sequence that is optimally aligned with a reference sequence, a nucleotide or amino acid residue “corresponds to” the position in the reference sequence with which the nucleotide or residue is paired in the alignment. The “position” is denoted by a number that sequentially identifies each nucleotide in the reference nucleotide sequence based on its position relative to the 5′ end or each amino acid in the reference amino acid sequence based on its position relative to the N-terminus. Owing to deletions, insertion, truncations, fusions, etc., that must be taken into account when determining an optimal alignment, in general the nucleotide position or amino acid residue number in a test sequence as determined by simply counting from the 5′ end or N-terminus will not necessarily be the same as the number of its corresponding position in the reference sequence. For example, in a case where there is a deletion in an aligned test sequence, there will be no nucleotide or amino acid that corresponds to a position in the reference sequence at the site of deletion. Where there is an insertion in an aligned reference sequence, that insertion will not correspond to any nucleotide or amino acid position in the reference sequence. In the case of truncations or fusions there can be stretches of nucleotides or amino acids in either the reference or aligned sequence that do not correspond to any nucleotide or amino acid in the corresponding sequence.

### VII. RGN Systems and Ribonucleoprotein Complexes for Binding a Target Sequence of Interest and Methods of Making the Same

The present disclosure provides a RGN system for binding a target sequence in a mutant HTT allele. As used herein, an RGN system comprises at least one RGN polypeptide or a polynucleotide comprising a nucleotide sequence encoding the RGN polypeptide and one or more guide RNAs capable of forming a complex with the RGN polypeptide (ribonucleoprotein (RNP) complex). The RGN systems comprise: a) one or more guide RNAs, or one or more polynucleotides comprising one or more nucleotide sequences encoding the one or more guide RNAs; and b) an RGN polypeptide or a polynucleotide comprising a nucleotide sequence encoding the RGN polypeptide, wherein the one or more guide RNAs are capable of forming a complex with the RGN polypeptide to direct said RGN polypeptide to bind to the target sequence in a mutant HTT allele. The guide RNA hybridizes to the target strand of a target sequence in a mutant HTT allele and also forms a complex with the RGN polypeptide, thereby directing the RGN polypeptide to bind to the target sequence. In some embodiments, the target sequence comprises a nucleotide sequence set forth as any one of SEQ ID NOs: 75-79, and 130. In some embodiments, the RGN is capable of recognizing a consensus PAM sequence set forth as NNNNCC, NNRYA, NNGRR, or NNGG. In some embodiments, the RGN comprises an amino acid sequence set forth as any one of SEQ ID NOs: 3, 7, 11, and 15, or an active variant or fragment thereof. In some embodiments, the RGN comprises an amino acid sequence having at least 80%, at least 85%, at least 90%, at least 95%, or more sequence identity to any one of SEQ ID NOs: 3, 7, 11, and 15. In some embodiments, the guide RNA comprises a CRISPR repeat comprising the nucleotide sequence set forth as any one of SEQ ID NOs: 4, 8, 12, 16, and 106, or an active variant or fragment thereof. In some embodiments, the guide RNA comprises a tracrRNA comprising any one of the nucleotide sequences set forth as any one of SEQ ID NOs: 5, 9, 13, 17, 107, and 120, or an active variant or fragment thereof. In some embodiments, the guide RNA comprises an sgRNA comprising any one of the nucleotide sequences set forth as any one of SEQ ID NOs: 6, 10, 14, 18, and 25-29, or an active variant or fragment thereof. The guide RNA of the system can be a single guide RNA or a dual-guide RNA. In some embodiments, the system comprises an RNA-guided nuclease that is heterologous to the guide RNA, wherein the RGN and guide RNA are not found complexed to one another (i.e., bound to one another) in nature.

In some embodiments, the PAM-interacting domain of the RGN polypeptide has the sequence set forth as SEQ ID NO: 133 and recognizes the PAM sequence NNNNCC. For example, an RGN polypeptide having at least 80%, at least 85%, at least 90%, at least 95%, or 100% sequence identity to SEQ ID NO: 3 comprises a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 133 and recognizes the PAM sequence NNNNCC. In some embodiments, an RGN polypeptide having at least 80%, at least 85%, at least 90%, at least 95%, or 100% sequence identity to SEQ ID NO: 3 comprises a PAM-interacting domain having an amino acid sequence having at least 95% sequence identity to SEQ ID NO: 133 and recognizes the PAM sequence NNNNCC. In some embodiments, an RGN polypeptide having at least 80%, at least 85%, at least 90%, at least 95%, or 100% sequence identity to SEQ ID NO: 3 comprises a PAM-interacting domain having the sequence set forth as SEQ ID NO: 133 and recognizes the PAM sequence NNNNCC.

In some embodiments, the PAM-interacting domain of the RGN polypeptide has the sequence set forth as SEQ ID NO: 134 and recognizes the PAM sequence NNRYA. For example, an RGN polypeptide having at least 80%, at least 85%, at least 90%, at least 95%, or 100% sequence identity to SEQ ID NO: 7 comprises a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 134 and recognizes the PAM sequence NNRYA. In some embodiments, an RGN polypeptide having at least 80%, at least 85%, at least 90%, at least 95%, or 100% sequence identity to SEQ ID NO: 7 comprises a PAM-interacting domain having an amino acid sequence having at least 95% sequence identity to SEQ ID NO: 134 and recognizes the PAM sequence NNRYA. In some embodiments, an RGN polypeptide having at least 80%, at least 85%, at least 90%, at least 95%, or 100% sequence identity to SEQ ID NO: 7 comprises a PAM-interacting domain having the sequence set forth as SEQ ID NO: 134 and recognizes the PAM sequence NNRYA.

In some embodiments, the PAM-interacting domain of the RGN polypeptide has the sequence set forth as SEQ ID NO: 135 and recognizes the PAM sequence NNGRR. For example, an RGN polypeptide having at least 80%, at least 85%, at least 90%, at least 95%, or 100% sequence identity to SEQ ID NO: 11 comprises a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 135 and recognizes the PAM sequence NNGRR. In some embodiments, an RGN polypeptide having at least 80%, at least 85%, at least 90%, at least 95%, or 100% sequence identity to SEQ ID NO: 11 comprises a PAM-interacting domain having an amino acid sequence having at least 95% sequence identity to SEQ ID NO: 135 and recognizes the PAM sequence NNGRR. In some embodiments, an RGN polypeptide having at least 80%, at least 85%, at least 90%, at least 95%, or 100% sequence identity to SEQ ID NO: 11 comprises a PAM-interacting domain having the sequence set forth as SEQ ID NO: 135 and recognizes the PAM sequence NNGRR.

In some embodiments, the PAM-interacting domain of the RGN polypeptide has the sequence set forth as SEQ ID NO: 136 and recognizes the PAM sequence NNGG. For example, an RGN polypeptide having at least 80%, at least 85%, at least 90%, at least 95%, or 100% sequence identity to SEQ ID NO: 15 comprises a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 136 and recognizes the PAM sequence NNGG. In some embodiments, an RGN polypeptide having at least 80%, at least 85%, at least 90%, at least 95%, or 100% sequence identity to SEQ ID NO: 15 comprises a PAM-interacting domain having an amino acid sequence having at least 95% sequence identity to SEQ ID NO: 136 and recognizes the PAM sequence NNGG. In some embodiments, an RGN polypeptide having at least 80%, at least 85%, at least 90%, at least 95%, or 100% sequence identity to SEQ ID NO: 15 comprises a PAM-interacting domain having the sequence set forth as SEQ ID NO: 136 and recognizes the PAM sequence NNGG.

The presently disclosed RGN systems can include an RGN polypeptide that comprises at least one nuclease domain, each of which is responsible for cleaving a single strand of a nucleic acid molecule. The nuclease domain can comprise a RuvC or an HNH domain. For example, an RGN polypeptide having at least 80%, at least 85%, at least 90%, at least 95%, or 100% sequence identity to SEQ ID NO: 3 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a nuclease domain having an amino acid sequence having at least 95% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a nuclease domain having the sequence set forth as any one of SEQ ID NOs: 143, 144, 145, and 146.

An RGN polypeptide of the present disclosure having at least 80%, at least 85%, at least 90%, at least 95%, or 100% sequence identity to SEQ ID NO: 7 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a nuclease domain having an amino acid sequence having at least 95% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a nuclease domain having the sequence set forth as any one of SEQ ID NOs: 147, 148, 149, and 150.

An RGN polypeptide of the present disclosure having at least 80%, at least 85%, at least 90%, at least 95%, or 100% sequence identity to SEQ ID NO: 11 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a nuclease domain having an amino acid sequence having at least 95% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a nuclease domain having the sequence set forth as any one of SEQ ID NOs: 151, 152, 153, and 154.

An RGN polypeptide having at least 80%, at least 85%, at least 90%, at least 95%, or 100% sequence identity to SEQ ID NO: 15 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a nuclease domain having an amino acid sequence having at least 95% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a nuclease domain having the sequence set forth as any one of SEQ ID NOs: 155, 156, 157, and 158.

The presently disclosed RGN systems can include an RGN polypeptide that comprises a PAM-interacting domain that contributes to the recognition of and binding to a PAM site and further comprises at least one nuclease domain, each of which nuclease domain is responsible for cleaving a single strand of a nucleic acid molecule. For example, an RGN polypeptide having at least 80%, at least 85%, at least 90%, at least 95%, or 100% sequence identity to SEQ ID NO: 3 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 133 and recognizes the PAM sequence NNNNCC, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a PAM-interacting domain having an amino acid sequence having at least 95% sequence identity to SEQ ID NO: 133 and recognizes the PAM sequence NNNNCC, and can further comprise a nuclease domain having an amino acid sequence having at least 95% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a PAM-interacting domain having the sequence set forth as SEQ ID NO: 133 and recognizes the PAM sequence NNNNCC, and can further comprise a nuclease domain having the amino acid sequence set forth as any one of SEQ ID NOs: 143, 144, 145, and 146.

An RGN polypeptide of the present disclosure having at least 80%, at least 85%, at least 90%, at least 95%, or 100% sequence identity to SEQ ID NO: 7 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 134 and recognize the PAM sequence NNRYA, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a PAM-interacting domain having an amino acid sequence having at least 95% sequence identity to SEQ ID NO: 134 and recognizes the PAM sequence NNRYA, and can further comprise a nuclease domain having an amino acid sequence having at least 95% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a PAM-interacting domain having the sequence set forth as SEQ ID NO: 134 and recognizes the PAM sequence NNRYA, and can further comprise a nuclease domain having the amino acid sequence set forth as any one of SEQ ID NOs: 147, 148, 149, and 150.

An RGN polypeptide of the present disclosure having at least 80%, at least 85%, at least 90%, at least 95%, or 100% sequence identity to SEQ ID NO: 11 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 135 and recognize the PAM sequence NNGRR, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a PAM-interacting domain having an amino acid sequence having at least 95% sequence identity to SEQ ID NO: 135 and recognizes the PAM sequence NNGRR, and can further comprise a nuclease domain having an amino acid sequence having at least 95% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a PAM-interacting domain having the sequence set forth as SEQ ID NO: 135 and recognizes the PAM sequence NNGRR, and can further comprise a nuclease domain having the amino acid sequence set forth as any one of SEQ ID NOs: 151, 152, 153, and 154.

An RGN polypeptide of the present disclosure having at least 80%, at least 85%, at least 90%, at least 95%, or 100% sequence identity to SEQ ID NO: 15 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 136 and recognize the PAM sequence NNGG, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a PAM-interacting domain having an amino acid sequence having at least 95% sequence identity to SEQ ID NO: 136 and recognizes the PAM sequence NNGG, and can further comprise a nuclease domain having an amino acid sequence having at least 95% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a PAM-interacting domain having the sequence set forth as SEQ ID NO: 136 and recognizes the PAM sequence NNGG, and can further comprise a nuclease domain having the amino acid sequence set forth as any one of SEQ ID NOs: 155, 156, 157, and 158.

The system for binding a target sequence of interest provided herein can be a ribonucleoprotein complex, which is at least one molecule of an RNA bound to at least one protein. The ribonucleoprotein complexes provided herein comprise at least one guide RNA as the RNA component and an RNA-guided nuclease as the protein component. Such ribonucleoprotein complexes can be purified from a cell or organism that naturally expresses an RGN polypeptide and has been engineered to express a particular guide RNA that is specific for a target sequence of interest (e.g., a target sequence in a mutant HTT allele). Alternatively, the ribonucleoprotein complex can be purified from a cell or organism that has been transformed with polynucleotides (e.g., an mRNA) that encode an RGN polypeptide and a guide RNA and cultured under conditions to allow for the expression of the RGN polypeptide and guide RNA. In some embodiments, the ribonucleoprotein complex is purified from a cell or organism that has been transformed with a polynucleotide (e.g., an mRNA) that encodes an RGN polypeptide and wherein a synthetically derived gRNA has been introduced. Thus, methods are provided for making an RGN polypeptide or an RGN ribonucleoprotein complex. Such methods comprise culturing a cell comprising a nucleotide sequence encoding an RGN polypeptide, and in some embodiments a nucleotide sequence encoding a guide RNA, under conditions in which the RGN polypeptide (and in some embodiments, the guide RNA) is expressed. The RGN polypeptide or RGN ribonucleoprotein can then be purified from a lysate of the cultured cells. In some embodiments, the nucleotide sequence encoding an RGN polypeptide includes a mRNA (messenger RNA). In some embodiments, methods for assembling an RNP complex comprise combining one or more of the presently disclosed guide RNAs and one or more of the presently disclosed RGN polypeptides under conditions suitable for formation of the RNP complex.

Methods for purifying an RGN polypeptide or RGN ribonucleoprotein complex from a lysate of a biological sample are known in the art (e.g., size exclusion and/or affinity chromatography, 2D-PAGE, HPLC, reversed-phase chromatography, immunoprecipitation). In particular methods, the RGN polypeptide is recombinantly produced and comprises a purification tag to aid in its purification, including but not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG (e.g., 3×FLAG tag), HA, nus, Softag 1, Softag 3, Strep, SBP, Glu-Glu, HSV, KT3, S, S1, T7, V5, VSV-G, 6×His, 10×His, biotin carboxyl carrier protein (BCCP), and calmodulin. Generally, the tagged RGN polypeptide or RGN ribonucleoprotein complex is purified using immobilized metal affinity chromatography. It will be appreciated that other similar methods known in the art may be used, including other forms of chromatography or for example immunoprecipitation, either alone or in combination.

An “isolated” or “purified” polypeptide, or biologically active portion thereof, is substantially or essentially free from components that normally accompany or interact with the polypeptide as found in its naturally occurring environment. Thus, an isolated or purified polypeptide is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals. Similarly, an “isolated” polynucleotide or nucleic acid molecule is removed from its naturally occurring environment. An isolated polynucleotide is substantially free of chemical precursors or other chemicals when chemically synthesized or has been removed from a genomic locus via the breaking of phosphodiester bonds. An isolated polynucleotide can be part of a vector, a composition of matter or can be contained within a cell so long as the cell is not the original environment of the polynucleotide.

Particular methods provided herein for binding and/or cleaving a target sequence of interest involve the use of an in vitro assembled RGN ribonucleoprotein complex. In vitro assembly of an RGN ribonucleoprotein complex can be performed using any method known in the art in which an RGN polypeptide is contacted with a guide RNA under conditions to allow for binding of the RGN polypeptide to the guide RNA. As used herein, “contact”, contacting”, “contacted,” refer to placing the components of a desired reaction together under conditions suitable for carrying out the desired reaction. The RGN polypeptide can be purified from a biological sample, cell lysate, or culture medium, produced via in vitro translation, or chemically synthesized. The guide RNA can be purified from a biological sample, cell lysate, or culture medium, transcribed in vitro, or chemically synthesized. The RGN polypeptide and guide RNA can be brought into contact in solution (e.g., buffered saline solution) to allow for in vitro assembly of the RGN ribonucleoprotein complex.

Some aspects of this disclosure provide kits comprising one or more elements of an RGN system described herein, including: guide RNAs (i.e. crRNAs, tracrRNAs, and/or sgRNAs), RGNs, and/or polynucleotides encoding the same; cells; and complete RGN systems, and in some embodiments another type of nuclease. In some embodiments, the kit includes suitable reagents, buffers, and/or instructions for using one or more elements of an RGN system, e.g., for in vitro or in vivo nucleic acid editing. Reagents may be provided in any suitable container, such as a vial, a bottle, or a tube. Reagents may be used in a process utilizing one or more of the elements of an RGN system. For example, restriction enzymes may be included for cloning of a polynucleotide encoding an RGN or a guide RNA into a vector. In some embodiments, the kit includes instructions regarding the design and use of suitable guide RNAs (i.e. crRNAs, tracrRNAs, and/or sgRNAs) for targeted editing of a nucleic acid sequence. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10.

A kit including one or more elements of an RGN system of the disclosure has utility in a wide variety of applications including modifying (e.g., deleting, inserting, translocating, inactivating, activating) a target polynucleotide in a multiplicity of cell types. As such, kits including one or more elements of an RGN system of the disclosure may be useful in, for example, gene therapy, drug screening, disease diagnosis, and prognosis.

In some embodiments, a kit of the disclosure includes a pharmaceutical kit including a pharmaceutical composition described herein. In some embodiments, a pharmaceutical kit may include: (a) a container containing a composition of the disclosure in lyophilized form and (b) a second container containing a pharmaceutically acceptable diluent (e.g., sterile water) for injection. The pharmaceutically acceptable diluent can be used for reconstitution or dilution of the lyophilized compound of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration.

### VII. Methods of Binding, Cleaving, and or Modifying a Target Sequence

The present disclosure provides methods for binding, cleaving, and/or modifying (i.e., editing) a target sequence in a mutant HTT allele. The methods include introducing an RGN system comprising at least one guide RNA or a polynucleotide encoding the same, and at least one RGN polypeptide or a polynucleotide encoding the same into a cell comprising the target sequence. In some embodiments, the delivery is ex vivo, and the cell comprising the target sequence can be a stem cell, a zygote, an embryonic cell, or a gamete. In some embodiments, the stem cell is an induced pluripotent stem cell (iPSC) or a mesenchymal stem cell (MSC). In some embodiments, the methods include delivering an RGN system in vivo, and the cell comprising the target sequence is in vivo. In some embodiments, the target sequence in a mutant HTT allele has a nucleotide sequence set forth as any one of SEQ ID NOs: 75-79, and 130. In some embodiments, the RGN is capable of recognizing a consensus PAM sequence set forth as any one of NNNNCC, NNRYA, NNGRR, and NNGG. In some embodiments, the RGN comprises an amino acid sequence set forth as any one of SEQ ID NOs: 3, 7, 11, and 15, or an active variant or fragment thereof. The guide RNA of the system can be a single guide RNA or a dual-guide RNA.

The RGN can comprise an amino acid sequence having at least 80%, at least 85%, at least 90%, at least 95%, or more sequence identity to SEQ ID NO: 3. In some embodiments, the guide RNA comprises a CRISPR repeat comprising the nucleotide sequence set forth as SEQ ID NO: 4, or an active variant or fragment thereof. In embodiments, the guide RNA comprises a tracrRNA comprising the nucleotide sequences set forth as SEQ ID NO: 5, or an active variant or fragment thereof. In embodiments, the guide RNA comprises an sgRNA comprising the nucleotide sequence set forth as SEQ ID NO: 27 or 28, or an active variant or fragment thereof. In some embodiments, the PAM-interacting domain of the RGN polypeptide has the sequence set forth as SEQ ID NO: 133 and recognizes the PAM sequence NNNNCC. For example, an RGN polypeptide having at least 80%, at least 85%, at least 90%, at least 95%, or 100% sequence identity to SEQ ID NO: 3 comprises a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 133 and recognizes the PAM sequence NNNNCC. In some embodiments, an RGN polypeptide having at least 80%, at least 85%, at least 90%, at least 95%, or 100% sequence identity to SEQ ID NO: 3 comprises a PAM-interacting domain having an amino acid sequence having at least 95% sequence identity to SEQ ID NO: 133 and recognizes the PAM sequence NNNNCC. In some embodiments, an RGN polypeptide having at least 80%, at least 85%, at least 90%, at least 95%, or 100% sequence identity to SEQ ID NO: 3 comprises a PAM-interacting domain having the sequence set forth as SEQ ID NO: 133 and recognizes the PAM sequence NNNNCC.

The RGN can comprise an amino acid sequence having at least 80%, at least 85%, at least 90%, at least 95%, or more sequence identity to SEQ ID NO: 7. In some embodiments, the guide RNA comprises a CRISPR repeat comprising the nucleotide sequence set forth as SEQ ID NO: 8 or 106, or an active variant or fragment thereof. In embodiments, the guide RNA comprises a tracrRNA comprising the nucleotide sequences set forth as SEQ ID NO: 9 or 107, or an active variant or fragment thereof. In embodiments, the guide RNA comprises an sgRNA comprising the nucleotide sequence set forth as SEQ ID NO: 25 or 26, or an active variant or fragment thereof. In some embodiments, the PAM-interacting domain of the RGN polypeptide has the sequence set forth as SEQ ID NO: 134 and recognizes the PAM sequence NNRYA. For example, an RGN polypeptide having at least 90%, at least 95%, or 100% sequence identity to SEQ ID NO: 7 comprises a PAM-interacting domain having an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 134 and recognizes the PAM sequence NNRYA. In some embodiments, an RGN polypeptide having at least 80%, at least 85%, at least 90%, at least 95%, or 100% sequence identity to SEQ ID NO: 7 comprises a PAM-interacting domain having an amino acid sequence having at least 95% sequence identity to SEQ ID NO: 134 and recognizes the PAM sequence NNRYA. In some embodiments, an RGN polypeptide having at least 80%, at least 85%, at least 90%, at least 95%, or 100% sequence identity to SEQ ID NO: 7 comprises a PAM-interacting domain having the sequence set forth as SEQ ID NO: 134 and recognizes the PAM sequence NNRYA.

The RGN can comprise an amino acid sequence having at least 80%, at least 85%, at least 90%, at least 95%, or more sequence identity to SEQ ID NO: 11. In some embodiments, the guide RNA comprises a CRISPR repeat comprising the nucleotide sequence set forth as SEQ ID NO: 12, or an active variant or fragment thereof. In embodiments, the guide RNA comprises a tracrRNA comprising the nucleotide sequences set forth as SEQ ID NO: 13 or 120, or an active variant or fragment thereof. In embodiments, the guide RNA comprises an sgRNA comprising the nucleotide sequence set forth as SEQ ID NO: 29, or an active variant or fragment thereof. In some embodiments, the PAM-interacting domain of the RGN polypeptide has the sequence set forth as SEQ ID NO: 135 and recognizes the PAM sequence NNGRR. For example, an RGN polypeptide having at least 80%, at least 85%, at least 90%, at least 95%, or 100% sequence identity to SEQ ID NO: 11 comprises a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 135 and recognizes the PAM sequence NNGRR. In some embodiments, an RGN polypeptide having at least 80%, at least 85%, at least 90%, at least 95%, or 100% sequence identity to SEQ ID NO: 11 comprises a PAM-interacting domain having an amino acid sequence having at least 95% sequence identity to SEQ ID NO: 135 and recognizes the PAM sequence NNGRR. In some embodiments, an RGN polypeptide having at least 80%, at least 85%, at least 90%, at least 95%, or 100% sequence identity to SEQ ID NO: 11 comprises a PAM-interacting domain having the sequence set forth as SEQ ID NO: 135 and recognizes the PAM sequence NNGRR.

The RGN can comprise an amino acid sequence having at least 80%, at least 85%, at least 90%, at least 95%, or more sequence identity to SEQ ID NO: 15. In some embodiments, the guide RNA comprises a CRISPR repeat comprising the nucleotide sequence set forth as SEQ ID NO: 16, or an active variant or fragment thereof. In embodiments, the guide RNA comprises a tracrRNA comprising the nucleotide sequences set forth as SEQ ID NO: 17, or an active variant or fragment thereof. In some embodiments, the PAM-interacting domain of the RGN polypeptide has the sequence set forth as SEQ ID NO: 136 and recognizes the PAM sequence NNGG. For example, an RGN polypeptide having at least 80%, at least 85%, at least 90%, at least 95%, or 100% sequence identity to SEQ ID NO: 15 comprises a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 136 and recognizes the PAM sequence NNGG. In some embodiments, an RGN polypeptide having at least 80%, at least 85%, at least 90%, at least 95%, or 100% sequence identity to SEQ ID NO: 15 comprises a PAM-interacting domain having an amino acid sequence having at least 95% sequence identity to SEQ ID NO: 136 and recognizes the PAM sequence NNGG. In some embodiments, an RGN polypeptide having at least 80%, at least 85%, at least 90%, at least 95%, or 100% sequence identity to SEQ ID NO: 15 comprises a PAM-interacting domain having the sequence set forth as SEQ ID NO: 136 and recognizes the PAM sequence NNGG.

The presently disclosed RGN systems can include an RGN polypeptide that comprises at least one nuclease domain, each of which is responsible for cleaving a single strand of a nucleic acid molecule. The nuclease domain can comprise a RuvC or an HNH domain. For example, an RGN polypeptide having at least 80%, at least 85%, at least 90%, at least 95%, or 100% sequence identity to SEQ ID NO: 3 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a nuclease domain having an amino acid sequence having at least 95% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a nuclease domain having the sequence set forth as any one of SEQ ID NOs: 143, 144, 145, and 146.

An RGN polypeptide of the present disclosure having at least 80%, at least 85%, at least 90%, at least 95%, or 100% sequence identity to SEQ ID NO: 7 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a nuclease domain having an amino acid sequence having at least 95% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a nuclease domain having the sequence set forth as any one of SEQ ID NOs: 147, 148, 149, and 150.

An RGN polypeptide of the present disclosure having at least 80%, at least 85%, at least 90%, at least 95%, or 100% sequence identity to SEQ ID NO: 11 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a nuclease domain having an amino acid sequence having at least 95% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a nuclease domain having the sequence set forth as any one of SEQ ID NOs: 151, 152, 153, and 154.

An RGN polypeptide having at least 80%, at least 85%, at least 90%, at least 95%, or 100% sequence identity to SEQ ID NO: 15 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a nuclease domain having an amino acid sequence having at least 95% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a nuclease domain having the sequence set forth as any one of SEQ ID NOs: 155, 156, 157, and 158.

The presently disclosed RGN systems can include an RGN polypeptide that comprises a PAM-interacting domain that contributes to the recognition of and binding to a PAM site and further comprises at least one nuclease domain, each of which nuclease domain is responsible for cleaving a single strand of a nucleic acid molecule. For example, an RGN polypeptide having at least 80%, at least 85%, at least 90%, at least 95%, or 100% sequence identity to SEQ ID NO: 3 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 133 and recognizes the PAM sequence NNNNCC, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a PAM-interacting domain having an amino acid sequence having at least 95% sequence identity to SEQ ID NO: 133 and recognizes the PAM sequence NNNNCC, and can further comprise a nuclease domain having an amino acid sequence having at least 95% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a PAM-interacting domain having the sequence set forth as SEQ ID NO: 133 and recognizes the PAM sequence NNNNCC, and can further comprise a nuclease domain having the amino acid sequence set forth as any one of SEQ ID NOs: 143, 144, 145, and 146.

An RGN polypeptide of the present disclosure having at least 80%, at least 85%, at least 90%, at least 95%, or 100% sequence identity to SEQ ID NO: 7 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 134 and recognize the PAM sequence NNRYA, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a PAM-interacting domain having an amino acid sequence having at least 95% sequence identity to SEQ ID NO: 134 and recognizes the PAM sequence NNRYA, and can further comprise a nuclease domain having an amino acid sequence having at least 95% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a PAM-interacting domain having the sequence set forth as SEQ ID NO: 134 and recognizes the PAM sequence NNRYA, and can further comprise a nuclease domain having the amino acid sequence set forth as any one of SEQ ID NOs: 147, 148, 149, and 150.

An RGN polypeptide of the present disclosure having at least 80%, at least 85%, at least 90%, at least 95%, or 100% sequence identity to SEQ ID NO: 11 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 135 and recognize the PAM sequence NNGRR, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a PAM-interacting domain having an amino acid sequence having at least 95% sequence identity to SEQ ID NO: 135 and recognizes the PAM sequence NNGRR, and can further comprise a nuclease domain having an amino acid sequence having at least 95% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a PAM-interacting domain having the sequence set forth as SEQ ID NO: 135 and recognizes the PAM sequence NNGRR, and can further comprise a nuclease domain having the amino acid sequence set forth as any one of SEQ ID NOs: 151, 152, 153, and 154.

An RGN polypeptide of the present disclosure having at least 80%, at least 85%, at least 90%, at least 95%, or 100% sequence identity to SEQ ID NO: 15 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 136 and recognize the PAM sequence NNGG, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a PAM-interacting domain having an amino acid sequence having at least 95% sequence identity to SEQ ID NO: 136 and recognizes the PAM sequence NNGG, and can further comprise a nuclease domain having an amino acid sequence having at least 95% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a PAM-interacting domain having the sequence set forth as SEQ ID NO: 136 and recognizes the PAM sequence NNGG, and can further comprise a nuclease domain having the amino acid sequence set forth as any one of SEQ ID NOs: 155, 156, 157, and 158.

In certain embodiments, the RGN and/or guide RNA is heterologous to the cell to which the RGN and/or guide RNA (or polynucleotide(s) encoding at least one of the RGN and guide RNA) are introduced.

In embodiments wherein the method comprises delivering a polynucleotide encoding a guide RNA and/or an RGN polypeptide, the cell can then be cultured under conditions in which the guide RNA and/or RGN polypeptide are expressed. In some embodiments, the method comprises contacting a target nucleic acid molecule with an RGN ribonucleoprotein complex. In some embodiments, the method comprises introducing into a cell comprising a target nucleic acid molecule an RGN ribonucleoprotein complex. The RGN ribonucleoprotein complex can be one that has been purified from a biological sample, recombinantly produced and subsequently purified, or in vitro-assembled as described herein. In embodiments wherein the RGN ribonucleoprotein complex that is contacted with the target nucleic acid molecule or cell comprising the target nucleic acid molecule, has been assembled in vitro, the method can further comprise the in vitro assembly of the complex prior to contact with the target nucleic acid molecule or cell comprising the target nucleic acid molecule.

A purified or in vitro assembled RGN ribonucleoprotein complex can be introduced into a cell using any method known in the art, including, but not limited to electroporation. Alternatively, an RGN polypeptide and/or polynucleotide encoding or comprising the guide RNA can be introduced into a cell using any method known in the art (e.g., electroporation).

Upon delivery to or contact with the target nucleic acid molecule or cell comprising the target nucleic acid molecule, the guide RNA directs the RGN to bind to the target sequence within the target nucleic acid molecule in a sequence-specific manner. In those embodiments wherein the RGN has nuclease activity, the RGN polypeptide cleaves the target sequence upon binding. The target sequence can subsequently be modified (i.e., edited) via endogenous repair mechanisms, such as non-homologous end joining (NHEJ).

Methods to measure binding of an RGN polypeptide to a target sequence are known in the art and include chromatin immunoprecipitation assays, gel mobility shift assays, DNA pull-down assays, reporter assays, microplate capture and detection assays. Likewise, methods to measure cleavage or modification of a target nucleic acid molecule comprising a target sequence are known in the art and include in vitro or in vivo cleavage assays wherein cleavage is confirmed using PCR, sequencing, or gel electrophoresis, with or without the attachment of an appropriate label (e.g., radioisotope, fluorescent substance) to the target sequence to facilitate detection of degradation products. Alternatively, the nicking triggered exponential amplification reaction (NTEXPAR) assay can be used (see, e.g., Zhang et al. (2016) Chem. Sci. 7:4951-4957). In vivo cleavage can be evaluated using the Surveyor assay (Guschin et al. (2010)Methods Mol Biol 649:247-256).

The methods can involve the use of only one RGN and only one guide RNA. Single double-strand cleavage within or near an expanded trinucleotide repeat has been shown to lead to loss of the repeat region or reduction in its length, possibly due to destabilization of the repeat tracts (Richard et al., PLoS ONE (2014), 9(4):e95611; Mittelman et al., Proc Natl Acad Sci USA (2009), 106(24):9607-12; van Agtmaal et al. Mol Ther. 2017 Jan. 4; 25(1):24-43). In some embodiments, the RGN and its associated guide RNA recognizes a PAM that has been generated by a SNP in the mutant HTT allele, such that the mutant HTT allele is cleaved by the RGN system, potentially leading to a reduction in the level of mutant HTT protein and/or mutant HTT mRNA.

The methods can involve the use of a single type of RGN complexed with more than one guide RNA. In some embodiments, the methods involve the use of two types of RGNs, each complexed with a guide RNA. The more than one guide RNA can target different regions of a mutant HTT allele. For example, a first guide RNA can target 5′ proximal to the expanded trinucleotide repeat in the HTT gene and a second guide RNA can target 3′ proximal to the expanded trinucleotide repeat to allow for excision of the expanded trinucleotide repeat.

A double-stranded break introduced by an RGN polypeptide can be repaired by a non-homologous end-joining (NHEJ) repair process. Due to the error-prone nature of NHEJ, repair of the double-stranded break can result in a mutation of the target sequence. In certain embodiments, a “mutation” in reference to a nucleic acid molecule refers to a change in the nucleotide sequence of the nucleic acid molecule, which can be a deletion, insertion, or substitution of one or more nucleotides, or a combination thereof. In some embodiments, cleavage of the mutHTT allele leads to the introduction of INDELS (insertions and/or deletions) and early termination, leading to a reduction in mutHTT mRNA and/or protein levels. In some embodiments, cleavage of the mutHTT allele leads to the introduction of premature stop codons, leading to a reduction in mutHTT protein levels.

In some embodiments, the cell to which an RGN and/or guide RNA (or polynucleotide(s) encoding at least one of the RGN and guide RNA) are introduced has a mutant HTT allele comprising at least 27 CAG repeats in exon 1 (e.g., 27, 28, 29, 30, 31, 32, 33, 34, 35, 36, or more than 36 CAG repeats). The cell can comprise a mutant HTT allele comprising at least 36 CAG repeats in exon 1 (e.g., 36, 37, 38, 39, 40, or more than 40 CAG repeats). In other embodiments, the cell comprises a mutant HTT allele comprising at least 40 CAG repeats in exon 1 (e.g., 40, 41, 42, 43, 44, 45, 46, 47, 48, 49, 50, 51, 52, 53, 54, 55, 56, or more than 56 CAG repeats). The cell can also comprise a mutant HTT allele comprising at least 56 CAG repeats in exon 1 (e.g., 56, 57, 58, 59, 60, 61, 62, 63, 64, 65, 66, 67, 68, 69, 70, or more than 70 CAG repeats).

The methods can comprise use of an RGN polypeptide or RGN system that is not capable of cleaving a wild type HTT allele. An RGN polypeptide or RGN system that is not capable of cleaving a wild type HTT allele means that the RGN polypeptide or RGN system is not capable of cleaving a wild type HTT allele at all or cleaves at a negligible level, such that the level of wtHTT mRNA and/or wtHTT protein is insignificantly decreased where, for example, the wtHTT can maintain support of critical cellular and neural functions and/or no symptoms of Huntington's disease is present in an in vivo setting (i.e. in a subject heterozygous for the mutHTT allele and administered the RGN polypeptide or RGN system). In some embodiments, the RGN polypeptide or RGN system cleaves at a negligible level, such that the level of wtHTT mRNA and/or wtHTT protein is decreased 5% or less, 4% or less, 3% or less, 2% or less, 1% or less, 0.9% or less, 0.8% or less, 0.7% or less, 0.6% or less, 0.5% or less, 0.4% or less, 0.3% or less, 0.2% or less, or 0.1% or less, as compared to the level of wtHTT mRNA and/or wtHTT protein in vitro or in vivo where an RGN polypeptide or RGN system of the disclosure has not been introduced.

### IX. Cells Comprising a Polynucleotide Genetic Modification

Provided herein are cells and organisms comprising a target sequence in a mutant HTT allele that has been modified using a process mediated by an RGN, crRNA, tracrRNA, and/or sgRNA as described herein. The cell comprising the modified target sequence in the mutant HTT allele can be a Huntington Disease patient cell, a stem cell, a zygote, an embryonic cell, or a gamete. In some embodiments, the stem cell is an induced pluripotent stem cell (iPSC) or a mesenchymal stem cell (MSC). In some embodiments, the cell is derived from an induced pluripotent stem cell (iPSC) or a mesenchymal stem cell (MSC). In some embodiments, the cell comprising the modified target sequence in the mutant HTT allele is in vitro or ex vivo. In some embodiments, the cell comprising the modified target sequence in the mutant HTT allele is in vivo. Cells that have been modified (e.g., embryonic cell, zygote, gamete) may develop into an organism under appropriate conditions. The RGN introduced into a cell to modify a target sequence in a mutant HTT allele can recognize a consensus PAM sequence including any one of NNNNCC, NNRYA, NNGRR, and NNGG. In some embodiments, the target sequence in a mutant HTT allele has a nucleotide sequence set forth as any one of SEQ ID NOs: 75-79, and 130. The guide RNA of the system can be a single guide RNA or a dual-guide RNA.

The RGN can comprise an amino acid sequence having at least 80%, at least 85%, at least 90%, at least 95% or more sequence identity to SEQ ID NO: 3. In some embodiments, the guide RNA comprises a CRISPR repeat comprising the nucleotide sequence set forth as SEQ ID NO: 4, or an active variant or fragment thereof. In embodiments, the guide RNA comprises a tracrRNA comprising the nucleotide sequences set forth as SEQ ID NO: 5, or an active variant or fragment thereof. In embodiments, the guide RNA comprises an sgRNA comprising the nucleotide sequence set forth as SEQ ID NO: 27 or 28, or an active variant or fragment thereof. In some embodiments, the PAM-interacting domain of the RGN polypeptide has the sequence set forth as SEQ ID NO: 133 and recognizes the PAM sequence NNNNCC. For example, an RGN polypeptide having at least 80%, at least 85%, at least 90%, at least 95%, or 100% sequence identity to SEQ ID NO: 3 comprises a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 133 and recognizes the PAM sequence NNNNCC. In some embodiments, an RGN polypeptide having at least 80%, at least 85%, at least 90%, at least 95%, or 100% sequence identity to SEQ ID NO: 3 comprises a PAM-interacting domain having an amino acid sequence having at least 95% sequence identity to SEQ ID NO: 133 and recognizes the PAM sequence NNNNCC. In some embodiments, an RGN polypeptide having at least 80%, at least 85%, at least 90%, at least 95%, or 100% sequence identity to SEQ ID NO: 3 comprises a PAM-interacting domain having the sequence set forth as SEQ ID NO: 133 and recognizes the PAM sequence NNNNCC.

The RGN can comprise an amino acid sequence having at least 80%, at least 85%, at least 90%, at least 95%, or more sequence identity to SEQ ID NO: 7. In some embodiments, the guide RNA comprises a CRISPR repeat comprising the nucleotide sequence set forth as SEQ ID NO: 8 or 106, or an active variant or fragment thereof. In embodiments, the guide RNA comprises a tracrRNA comprising the nucleotide sequences set forth as SEQ ID NO: 9 or 107, or an active variant or fragment thereof. In embodiments, the guide RNA comprises an sgRNA comprising the nucleotide sequence set forth as SEQ ID NO: 25 or 26, or an active variant or fragment thereof. In some embodiments, the PAM-interacting domain of the RGN polypeptide has the sequence set forth as SEQ ID NO: 134 and recognizes the PAM sequence NNRYA. For example, an RGN polypeptide having at least 80%, at least 85%, at least 90%, at least 95%, or 100% sequence identity to SEQ ID NO: 7 comprises a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 134 and recognizes the PAM sequence NNRYA. In some embodiments, an RGN polypeptide having at least 80%, at least 85%, at least 90%, at least 95%, or 100% sequence identity to SEQ ID NO: 7 comprises a PAM-interacting domain having an amino acid sequence having at least 95% sequence identity to SEQ ID NO: 134 and recognizes the PAM sequence NNRYA. In some embodiments, an RGN polypeptide having at least 80%, at least 85%, at least 90%, at least 95%, or 100% sequence identity to SEQ ID NO: 7 comprises a PAM-interacting domain having the sequence set forth as SEQ ID NO: 134 and recognizes the PAM sequence NNRYA.

The RGN can comprise an amino acid sequence having at least 80%, at least 85%, at least 90%, at least 95%, or more sequence identity to SEQ ID NO: 11. In some embodiments, the guide RNA comprises a CRISPR repeat comprising the nucleotide sequence set forth as SEQ ID NO: 12, or an active variant or fragment thereof. In embodiments, the guide RNA comprises a tracrRNA comprising the nucleotide sequences set forth as SEQ ID NO: 13 or 120, or an active variant or fragment thereof. In embodiments, the guide RNA comprises an sgRNA comprising the nucleotide sequence set forth as SEQ ID NO: 29, or an active variant or fragment thereof. In some embodiments, the PAM-interacting domain of the RGN polypeptide has the sequence set forth as SEQ ID NO: 135 and recognizes the PAM sequence NNGRR. For example, an RGN polypeptide having at least 80%, at least 85%, at least 90%, at least 95%, or 100% sequence identity to SEQ ID NO: 11 comprises a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 135 and recognizes the PAM sequence NNGRR. In some embodiments, an RGN polypeptide having at least 80%, at least 85%, at least 90%, at least 95%, or 100% sequence identity to SEQ ID NO: 11 comprises a PAM-interacting domain having an amino acid sequence having at least 95% sequence identity to SEQ ID NO: 135 and recognizes the PAM sequence NNGRR. In some embodiments, an RGN polypeptide having at least 80%, at least 85%, at least 90%, at least 95%, or 100% sequence identity to SEQ ID NO: 11 comprises a PAM-interacting domain having the sequence set forth as SEQ ID NO: 135 and recognizes the PAM sequence NNGRR.

The RGN can comprise an amino acid sequence having at least 80%, at least 85%, at least 90%, at least 95%, or more sequence identity to SEQ ID NO: 15. In some embodiments, the guide RNA comprises a CRISPR repeat comprising the nucleotide sequence set forth as SEQ ID NO: 16, or an active variant or fragment thereof. In embodiments, the guide RNA comprises a tracrRNA comprising the nucleotide sequences set forth as SEQ ID NO: 17, or an active variant or fragment thereof. In some embodiments, the PAM-interacting domain of the RGN polypeptide has the sequence set forth as SEQ ID NO: 136 and recognizes the PAM sequence NNGG. For example, an RGN polypeptide having at least 80%, at least 85%, at least 90%, at least 95%, or 100% sequence identity to SEQ ID NO: 15 comprises a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 136 and recognizes the PAM sequence NNGG. In some embodiments, an RGN polypeptide having at least 80%, at least 85%, at least 90%, at least 95%, or 100% sequence identity to SEQ ID NO: 15 comprises a PAM-interacting domain having an amino acid sequence having at least 95% sequence identity to SEQ ID NO: 136 and recognizes the PAM sequence NNGG. In some embodiments, an RGN polypeptide having at least 80%, at least 85%, at least 90%, at least 95%, or 100% sequence identity to SEQ ID NO: 15 comprises a PAM-interacting domain having the sequence set forth as SEQ ID NO: 136 and recognizes the PAM sequence NNGG.

The presently disclosed RGN systems can include an RGN polypeptide that comprises at least one nuclease domain, each of which is responsible for cleaving a single strand of a nucleic acid molecule. The nuclease domain can comprise a RuvC or an HNH domain. For example, an RGN polypeptide having at least 80%, at least 85%, at least 90%, at least 95%, or 100% sequence identity to SEQ ID NO: 3 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a nuclease domain having an amino acid sequence having at least 95% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a nuclease domain having the sequence set forth as any one of SEQ ID NOs: 143, 144, 145, and 146.

An RGN polypeptide of the present disclosure having at least 80%, at least 85%, at least 90%, at least 95%, or 100% sequence identity to SEQ ID NO: 7 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a nuclease domain having an amino acid sequence having at least 95% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a nuclease domain having the sequence set forth as any one of SEQ ID NOs: 147, 148, 149, and 150.

An RGN polypeptide of the present disclosure having at least 80%, at least 85%, at least 90%, at least 95%, or 100% sequence identity to SEQ ID NO: 11 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a nuclease domain having an amino acid sequence having at least 95% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a nuclease domain having the sequence set forth as any one of SEQ ID NOs: 151, 152, 153, and 154.

An RGN polypeptide having at least 80%, at least 85%, at least 90%, at least 95%, or 100% sequence identity to SEQ ID NO: 15 can comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a nuclease domain having an amino acid sequence having at least 95% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a nuclease domain having the sequence set forth as any one of SEQ ID NOs: 155, 156, 157, and 158.

The presently disclosed RGN systems can include an RGN polypeptide that comprises a PAM-interacting domain that contributes to the recognition of and binding to a PAM site and further comprises at least one nuclease domain, each of which nuclease domain is responsible for cleaving a single strand of a nucleic acid molecule. For example, an RGN polypeptide having at least 80%, at least 85%, at least 90%, at least 95%, or 100% sequence identity to SEQ ID NO: 3 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 133 and recognizes the PAM sequence NNNNCC, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a PAM-interacting domain having an amino acid sequence having at least 95% sequence identity to SEQ ID NO: 133 and recognizes the PAM sequence NNNNCC, and can further comprise a nuclease domain having an amino acid sequence having at least 95% sequence identity to any one of SEQ ID NOs: 143, 144, 145, and 146. In some embodiments, an RGN polypeptide having at least 80%, at least 85%, at least 90%, at least 95%, or 100% sequence identity to SEQ ID NO: 3 comprises a PAM-interacting domain having the sequence set forth as SEQ ID NO: 133 and recognizes the PAM sequence NNNNCC, and can further comprise a nuclease domain having the amino acid sequence set forth as any one of SEQ ID NOs: 143, 144, 145, and 146.

An RGN polypeptide of the present disclosure having at least 80%, at least 85%, at least 90%, at least 95%, or 100% sequence identity to SEQ ID NO: 7 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 134 and recognize the PAM sequence NNRYA, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a PAM-interacting domain having an amino acid sequence having at least 95% sequence identity to SEQ ID NO: 134 and recognizes the PAM sequence NNRYA, and can further comprise a nuclease domain having an amino acid sequence having at least 95% sequence identity to any one of SEQ ID NOs: 147, 148, 149, and 150. In some embodiments, an RGN polypeptide having at least 80%, at least 85%, at least 90%, at least 95%, or 100% sequence identity to SEQ ID NO: 7 comprises a PAM-interacting domain having the sequence set forth as SEQ ID NO: 134 and recognizes the PAM sequence NNRYA, and can further comprise a nuclease domain having the amino acid sequence set forth as any one of SEQ ID NOs: 147, 148, 149, and 150.

An RGN polypeptide of the present disclosure having at least 80%, at least 85%, at least 90%, at least 95%, or 100% sequence identity to SEQ ID NO: 11 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 135 and recognize the PAM sequence NNGRR, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a PAM-interacting domain having an amino acid sequence having at least 95% sequence identity to SEQ ID NO: 135 and recognizes the PAM sequence NNGRR, and can further comprise a nuclease domain having an amino acid sequence having at least 95% sequence identity to any one of SEQ ID NOs: 151, 152, 153, and 154. In some embodiments, an RGN polypeptide having at least 80%, at least 85%, at least 90%, at least 95%, or 100% sequence identity to SEQ ID NO: 11 comprises a PAM-interacting domain having the sequence set forth as SEQ ID NO: 135 and recognizes the PAM sequence NNGRR, and can further comprise a nuclease domain having the amino acid sequence set forth as any one of SEQ ID NOs: 151, 152, 153, and 154.

An RGN polypeptide of the present disclosure having at least 80%, at least 85%, at least 90%, at least 95%, or 100% sequence identity to SEQ ID NO: 15 can comprise a PAM-interacting domain having an amino acid sequence having at least 90%, at least 91%, at least 92%, at least 93%, at least 94%, at least 95%, at least 96%, at least 97%, at least 98%, at least 99%, or 100% sequence identity to SEQ ID NO: 136 and recognize the PAM sequence NNGG, and can further comprise a nuclease domain having an amino acid sequence having at least 90%, at least 91%, at least 92%, at least 93%, at least 94%, at least 95%, at least 96%, at least 97%, at least 98%, at least 99%, or 100% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a PAM-interacting domain having an amino acid sequence having at least 95% sequence identity to SEQ ID NO: 136 and recognizes the PAM sequence NNGG, and can further comprise a nuclease domain having an amino acid sequence having at least 95% sequence identity to any one of SEQ ID NOs: 155, 156, 157, and 158. In some embodiments, an RGN polypeptide having at least 80%, at least 85%, at least 90%, at least 95%, or 100% sequence identity to SEQ ID NO: 15 comprises a PAM-interacting domain having the sequence set forth as SEQ ID NO: 136 and recognizes the PAM sequence NNGG, and can further comprise a nuclease domain having the amino acid sequence set forth as any one of SEQ ID NOs: 155, 156, 157, and 158.

The modified cells can be eukaryotic (e.g., mammalian). In some embodiments, cells that are modified by the presently disclosed methods include stem cells (e.g., induced pluripotent stem cells, mesenchymal stem cells), neuronal cells, and glial cells. A stem cell refers to a cell that is totipotent, pluripotent, or multipotent, and is capable of differentiating into one or more different cell types. The term “totipotent” refers to an ability of a cell to differentiate into any type of cell in a differentiated organism, as well as a cell of extra-embryonic materials such as placenta. The tern “pluripotent” refers to a cell line capable of differentiating into any terminally differentiated cell type. The term “multipotent” refers to a cell line capable of differentiating into at least two terminally differentiated cell types. The term “induced pluripotent stem cell” or “iPSC” refers to a type of pluripotent stem cell, similar to an embryonic stem cell, formed by the introduction of certain embryonic genes (such as a OCT4, SOX2, and KLF4 trans genes) (see, for example, Takahashi and Yamanaka Cell 126, 663-676 (2006), herein incorporated by reference) into a somatic (e.g., adult) cell. Examples of somatic cells include, but are not limited to, bone marrow cells, epithelial cells, fibroblast cells, hematopoietic cells, hepatic cells, intestinal cells, mesenchymal cells, myeloid precursor cells, neuronal cells, glial cells, and spleen cells. Alternatively, the iPSC can be produced by reprogramming a somatic cell to enter an embryonic stem cell-like state by being forced to express factors important for maintaining the “stemness” of embryonic stem cells (ESCs). Reprogramming factors may be expressed from expression cassettes comprised in one or more vectors, such as an integrating vector, a chromosomally non-integrating RNA viral vector, or an episomal vector, such as an EBV element-based system (Yu et al. (2009) Science, 324(5928):797-801). In some embodiments, reprogramming proteins or RNA (such as mRNA or miRNA) could be introduced directly into somatic cells by protein or RNA transfection (Yakubov et al. (2010) Biochemical and biophysical research communications, 394(1):189-193).

Mesenchymal stem cells (MSCs) can give rise to connective tissue, bone, cartilage, and cells in the circulatory and lymphatic systems. MSCs are found in the mesenchyme, the part of the embryonic mesoderm that contain loosely packed, fusiform, or stellate unspecialized cells. In some embodiments, MSCs include CD34⁻ stem cells. MSCs can be isolated from various sources including bone marrow, umbilical cord blood, (mobilized) peripheral blood, and adipose tissue (Horwitz et al. Clarification of the nomenclature for MSC: the International Society for Cellular Therapy position statement. Cytotherapy (2005) 7:393-395).

A stem cell (e.g., an iPSC or MSC) comprising a target sequence in the mutant HTT allele modified by the described RGN systems can be differentiated into a neuronal or glial cell. Differentiating a cell refers to changing the default cell type (genotype and/or phenotype) to a non-default cell type (genotype and/or phenotype). For example, differentiating a stem cell (e.g., an iPSC or MSC) refers to inducing the stem cell to divide into progeny cells with characteristics that are different from the stem cell (e.g., iPSC or MSC), such as genotype (i.e. change in gene expression as determined by genetic analysis such as a micro array) and/or phenotype (i.e. change in expression of a protein). One or more of small molecules, growth factor proteins, and other growth conditions can be used to promote the transition of an unspecialized state, e.g., that of a stem cell, into a more specialized cell fate (e.g., neuronal cell). In some embodiments, differentiation of a stem cell causes the stem cell to commit to a cellular pathway leading to a somatic cell. For example, factors to differentiate a stem cell into a neuronal cell may include: Wnt activators; SMAD inhibitors (e.g., Noggin peptide, SB-431542); neuronal growth factors (e.g., brain-derived neurotrophic factor, nerve-growth factor, glial-derived neurotrophic factor); and/or introduction of polynucleotides to express a neuronal gene (e.g., neurogenin-2, NeuroD1). The differentiation may comprise culturing pluripotent stem cells and/or progeny cells thereof in an adherent or suspension culture.

Neuronal cells that can be modified by a process utilizing an RGN polypeptide, crRNA, tracrRNA, and/or guide RNA as described herein can include neural progenitor cells, forebrain neuron progenitor cells, striatal neurons, medium spiny neurons, and cortical neurons. Non-neuronal brain cells that can be modified by a process utilizing an RGN polypeptide, crRNA, tracrRNA, and/or guide RNA as described herein include a glial cell. Glial cells can include microglia, astrocytes, and oligodendrocytes. In some embodiments, cells useful in the present disclosure include a mammalian cell or human cell present in putamen, caudate, striatum, cerebral cortex, globus pallidus, hippocampus, amygdala, thalamus, hypothalamus, subthalamic nucleus, substantia nigra, cerebellum, brainstem, or a combination thereof, of the brain.

Also provided are embryonic cells, zygotes, or gametes comprising a mutant HTT allele that has been modified by a process utilizing an RGN, crRNA, tracrRNA, and/or sgRNA as described herein. The modified cells and organisms can be heterozygous or homozygous for the modified mutant HTT allele. In some embodiments, the modified cells and organisms are heterozygous for the modified mutant HTT allele.

The chromosomal modification of a cell comprising a target sequence in a mutant HTT allele with an RGN system of the disclosure can result in downregulation of expression of the mutant HTT protein and/or mutant HTT mRNA. In some embodiments, the chromosomal modification results in a reduction or elimination of mutant HTT mRNA as compared to a level of mutant HTT mRNA in a cell that has not undergone chromosomal modification with the RGN system. In some embodiments, the chromosomal modification results in reduction or elimination of mutant HTT protein as compared to a level of mutant HTT protein in a cell that has not undergone chromosomal modification with the RGN system. Mutant HTT protein levels can be measured by assays including immunoassays that use antibodies that can distinguish between wild-type and mutant HTT protein (e.g., Western blot, ELISA, single-molecule counting immunoassay, immunoprecipitation assay combined with flow cytometry, time-resolved fluorescence energy transfer (TR-FRET)), and include the JESS capillary western blot assay described herein in the examples. Mutant HTT mRNA levels can be measured by, e.g., RT-qPCR or array-based methods.

### X. Pharmaceutical Compositions

Pharmaceutical compositions comprising: the presently disclosed crRNAs and active variants and fragments thereof, or polynucleotides encoding the same; the presently disclosed tracrRNAs and active variants and fragments thereof, or polynucleotides encoding the same; the presently disclosed sgRNAs and active variants and fragments thereof, or polynucleotides encoding the same; the presently disclosed RGN polypeptides and active variants and fragments thereof, or polynucleotides encoding the same; the presently disclosed RGN systems; or the presently disclosed RNP complexes comprising an RGN polypeptide and a gRNA; or the presently disclosed vectors (e.g., viral vectors); and a pharmaceutically acceptable cater are provided.

A pharmaceutical composition is a composition that is employed to prevent, reduce in intensity, cure or otherwise treat a target condition or disease that comprises an active ingredient (i.e., RGN polypeptides, RGN-encoding polynucleotides, gRNA, gRNA-encoding polynucleotides, RGN systems, RNP complexes, or vectors) and a pharmaceutically acceptable carrier.

As used herein, a “pharmaceutically acceptable cater” refers to a material that does not cause significant irritation to an organism and does not abrogate the activity and properties of the active ingredient (i.e., RGN polypeptides, RGN-encoding polynucleotides, gRNA, gRNA-encoding polynucleotides, RGN systems, RNP complexes, or vectors). Carriers must be of sufficiently high purity and of sufficiently low toxicity to render them suitable for administration to a subject being treated. The cater can be inert, or it can possess pharmaceutical benefits. In some embodiments, a pharmaceutically acceptable cater comprises one or more compatible solid or liquid filler, diluents or encapsulating substances which are suitable for administration to a human or other vertebrate animal. In some embodiments, the pharmaceutically acceptable cater is not naturally-occurring. In some embodiments, the pharmaceutically acceptable carrier and the active ingredient are not found together in nature.

Pharmaceutical compositions used in the presently disclosed methods can be formulated with suitable carriers, excipients, and other agents that provide suitable transfer, delivery, tolerance, and the like. A multitude of appropriate formulations are known to those skilled in the art. See, e.g., Remington, The Science and Practice of Pharmacy (21st ed. 2005). Suitable formulations include, for example, powders, pastes, ointments, jellies, waxes, oils, lipids, lipid (cationic or anionic) containing vesicles (such as LIPOFECTIN vesicles), lipid nanoparticles, DNA conjugates, anhydrous absorption pastes, oil-in-water and water-in-oil emulsions, emulsions carbowax (polyethylene glycols of various molecular weights), semi-solid gels, and semi-solid mixtures containing carbowax. Pharmaceutical compositions for oral or parenteral use may be prepared into dosage forms in a unit dose suited to fit a dose of the active ingredients. Such dosage forms in a unit dose include, for example, tablets, pills, capsules, injections (ampoules), suppositories, etc.

A pharmaceutical composition comprising the active ingredient (i.e., RGN polypeptides, RGN-encoding polynucleotides, gRNA, gRNA-encoding polynucleotides, RGN systems, or RNP complexes, or vectors) can be emulsified or presented as a liposome composition, provided that the emulsification procedure does not adversely affect the active ingredient or patient.

Additional agents included in a pharmaceutical composition can include pharmaceutically acceptable salts. Pharmaceutically acceptable salts include the acid addition salts (formed with the free amino groups of the polypeptide) that are formed with inorganic acids, such as, for example, hydrochloric or phosphoric acids, or such organic acids as acetic, tartaric, mandelic and the like. Salts formed with the free carboxyl groups can also be derived from inorganic bases, such as, for example, sodium, potassium, ammonium, calcium or ferric hydroxides, and such organic bases as isopropylamine, trimethylamine, 2-ethylamino ethanol, histidine, procaine and the like.

Physiologically tolerable and pharmaceutically acceptable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, sucrose, mannose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition can depend on the nature of the disorder or condition, and can be determined by standard clinical techniques.

In some embodiments, the pharmaceutical composition comprises one or molecules having surfactant properties to allow them to interact with biological membranes, for example pluronics or poloxamers such as PLURONIC F68 (poloxamer 188, P188). In some embodiments, the pharmaceutical composition comprises between 0.001% and 0.1% poloxamer. In some embodiments, the pharmaceutical composition comprises about 0.001% poloxamer.

The presently disclosed RGN polypeptides, guide RNAs, RGN systems, polynucleotides encoding the same, RNP complexes, or vectors can be formulated with pharmaceutically acceptable excipients such as carriers, solvents, stabilizers, adjuvants, diluents, etc., depending upon the particular mode of administration and dosage form. In some embodiments, these pharmaceutical compositions are formulated to achieve a physiologically compatible pH, and range from a pH of about 3 to a pH of about 11, about pH 3 to about pH 7, depending on the formulation and route of administration. In some embodiments, the pH can be adjusted to a range from about pH 5.0 to about pH 8. In some embodiments, the compositions can comprise a therapeutically effective amount of at least one active ingredient as described herein (i.e., RGN polypeptides, RGN-encoding polynucleotides, gRNA, gRNA-encoding polynucleotides, RGN systems, RNP complexes, or vectors), together with one or more pharmaceutically acceptable excipients. In some embodiments, the compositions comprise a combination of active ingredients described herein, or include a second active ingredient useful in the treatment or prevention of bacterial growth (for example and without limitation, anti-bacterial or anti-microbial agents), or include a combination of reagents of the present disclosure.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

In some embodiments, the formulations are provided in unit-dose or multi-dose containers, for example sealed ampules and vials, and may be stored in a freeze-dried (lyophilized) condition requiring the addition of the sterile liquid carrier, for example, saline, water-for-injection, a semi-liquid foam, or gel, immediately prior to use. Extemporaneous injection solutions and suspensions may be prepared from sterile powders, granules and tablets of the kind previously described. In some embodiments, the active ingredient is dissolved in a buffered liquid solution that is frozen in a unit-dose or multi-dose container and later thawed for injection or kept/stabilized under refrigeration until use.

The therapeutic agent(s) may be contained in controlled release systems. In order to prolong the effect of a drug, it often is desirable to slow the absorption of the drug from subcutaneous, intrathecal, or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. In some embodiments, the use of a long-term sustained release implant may be particularly suitable for treatment of chronic conditions. Long-term sustained release implants are well-known to those of ordinary skill in the art.

The therapeutic agent(s), such as the presently disclosed crRNAs and active variants and fragments thereof, or polynucleotides encoding the same; the presently disclosed tracrRNAs and active variants and fragments thereof, or polynucleotides encoding the same; the presently disclosed sgRNAs and active variants and fragments thereof, or polynucleotides encoding the same; the presently disclosed RGN polypeptides and active variants and fragments thereof, or polynucleotides encoding the same; the presently disclosed RGN systems; the presently disclosed RNP complexes comprising an RGN polypeptide and a gRNA; or the presently disclosed vectors (e.g., viral vectors), can be isolated or substantially or essentially free from components that normally accompany or interact with the therapeutic agent in its naturally occurring environment, or from chemical precursors or other chemicals used to synthesize the therapeutic agent, or from culture medium when produced by recombinant techniques, or from endotoxin and/or associated pyrogenic substances. Endotoxins include toxins that are confined within the interior of a microorganism and are released only upon breakdown or death of the microorganism. Pyrogenic substances also include fever-inducing thermostable substances (glycoproteins) from the outer membrane of bacteria and other microorganisms. Both substances can cause fever, hypotension and shock if administered to humans. Due to potentially harmful effects, even small amounts of endotoxins must be removed from an intravenously administered pharmaceutical drug solution. The U.S. Food and Drug Administration (“FDA”) has set an upper limit of 5 Endotoxin Units (EU)/dose/kg body weight over a one hour period for intravenous Drug use (The United States pharmaceutical Convention, pharmaceutical form 26 (1):223 (2000)). In certain particular aspects, the endotoxin and pyrogen levels in the composition are less than about 1 EU/mg, or less than about 0.1 EU/mg, or less than about 0.01 EU/mg, or less than about 0.001 EU/mg. In some embodiments, the endotoxin and pyrogen levels in the composition are 0.0138 EU/mg or less.

The therapeutic agent or pharmaceutical composition can have a purity of at least 80%, 85%, 90%, 95%, or greater. The therapeutic agent or pharmaceutical composition can have low or undetectable levels of endotoxin or other impurities.

The pharmaceutical composition may be frozen, refrigerated or stored at room temperature. Storage conditions may be below freezing, e.g., below about-10° C., or below about −20° C., or below about −40° C., or below about −70° C. Storage conditions are generally less than room temperature, e.g., less than about 32° C., or less than about 30° C., or less than about 27° C., or less than about 25° C., or less than about 20° C., or less than about 15° C. In some embodiments, the formulation is stored at 2° C.-8° C. For example, the formulation may be isotonic with blood or have an ionic strength that mimics physiological conditions.

In some embodiments, the pharmaceutical composition is stable under storage conditions. Stability can be measured using any suitable means in the art. Generally, a stable formulation is one that exhibits less than a 5% increase in degradation products or impurities. In some embodiments, the formulation is stable under storage conditions for at least about 1 month, at least about 2 months, at least about 3 months, at least about 4 months, at least about 5 months, at least about 6 months, at least about one year, or at least about 2 years or more. In some embodiments, the formulation is stable at 25° C. for at least about 1 month, at least about 2 months, at least about 3 months, at least about 4 months, at least about 5 months, at least about 6 months, or at least about one year or more.

When used for in vivo administration, the pharmaceutical compositions of the present disclosure should be sterile. The formulations of the present disclosure may be sterilized by a variety of sterilization methods including sterile filtration, irradiation, and the like. In one aspect, the formulation is filter sterilized with a pre-sterilized 0.22 micron filter. Sterile compositions for injection may be formulated according to conventional pharmaceutical Practice as described in “Remington: The Science & Practice of Pharmacy”, 21 st edition, Lippincott Williams & Wilkins, (2005).

### XI. Methods of Treating Huntington's Disease

Methods of treating Huntington's Disease (HD) in a subject in need thereof are provided herein. The methods comprise administering to a subject in need thereof a presently disclosed crRNA or polynucleotide encoding the same, a presently disclosed tracrRNA or polynucleotide encoding the same, a presently disclosed sgRNA or polynucleotides encoding the same, a presently disclosed RGN or polynucleotide encoding the same, a presently disclosed RGN system, a presently disclosed RNP complex, or a presently disclosed vector, or a pharmaceutical composition comprising any one of these. In some embodiments, the therapeutic composition can reduce or inhibit mutant HTT gene expression, reduce or inhibit mutant HTT protein production, and/or reduce or prevent one or more symptoms of HD in a subject in need thereof, such that HD is therapeutically treated.

In some embodiments, the treatment comprises in vivo gene editing by administering a presently disclosed RGN system, polynucleotide(s) encoding the same, a presently disclosed RNP complex, or a presently disclosed vector. In some embodiments, the treatment comprises ex vivo gene editing of a zygote, an embryonic cell, or a gamete to correct the genetic error at an early stage of life. In some embodiments, a therapeutic composition comprising an RGN system, polynucleotide(s) encoding the same, an RNP complex, or a presently disclosed vector are targeted to a subject's cells in vivo. In some embodiments, the cells targeted for gene editing of a mutant HTT allele include stem cells, neurons (e.g., medium spiny neurons, cortical neurons), and glial cells (e.g., astrocytes, oligodendrocytes, and microglia).

Huntington's disease (HD) is a monogenic, fatal neurodegenerative disease characterized by progressive chorea (involuntary movements), neuropsychiatric dysfunction, and cognitive dysfunction. Symptoms typically appear between the ages of 35-44 and life expectancy subsequent to onset is 10-25 years.

HD is known to be caused by a triplet cytosine-adenine-guanine (CAG) repeat expansion at the end of exon 1 of the huntingtin (HTT) gene. The CAG repeat encodes poly-glutamine in the N-terminus of the Huntingtin (HTT) protein. Normal HTT alleles contain 15-20 CAG repeats, while alleles containing 27-35 CAG repeats are considered to be intermediate alleles with little likelihood of developing a disease phenotype. HTT alleles containing 35 or more CAG repeats can be considered potentially HD causing alleles and confer risk for developing the disease. Alleles containing 36-39 CAG repeats are considered incompletely penetrant, and those individuals harboring those alleles may or may not develop the disease (or may develop symptoms later in life) while alleles containing 40 CAG repeats or more are considered completely penetrant. A Huntington's Disease Integrated Staging System (HD-ISS) has been developed that defines stages of the disease from birth to death and takes into account clinical criteria, clinical biomarkers, and functional assessments to classify subjects with HD (Tabrizi et al. Lancet Neurol 2022; 21: 632-644).

Juvenile Onset HD (JHD) is a form of HD that affects children and teenagers. Those individuals with juvenile onset HD (<21 years of age) are often found to have 60 or more CAG repeats. JHD symptoms include changes in personality, coordination, behavior, speech, or cognitive abilities. Physical changes occur too and include rigidity, leg stiffness, clumsiness, slowness of movement, tremors or myoclonus. In contrast to adult HD, seizures and rigidity are common, and chorea is uncommon. JHD has a more rapid progression rate as compared to adult HD, and death can occur within 10 years of onset.

The mutant HTT allele is usually inherited from one parent as a dominant trait. Any child born of a HD patient has a 50% chance of developing the disease if the other parent was not afflicted with the disorder. In some cases, a parent may have an intermediate HD allele and be asymptomatic while, due to repeat expansion, the child manifests the disease. In addition, the HD allele can also display a phenomenon known as anticipation wherein increasing severity or decreasing age of onset is observed over several generations due to the unstable nature of the repeat region during spermatogenesis.

This repeat expansion results in a mutant HTT protein that can form aggregates in cells, interfere with normal cellular functions, and/or have pathologic interactions with other molecules. Ultimately, the presence of mutant HTT protein leads to striatal neurodegeneration which progresses to widespread brain atrophy.

Trinucleotide expansion in the HTT gene leads to neuronal loss in the medium spiny gamma-aminobutyric acid (GABA) projection neurons in the striatum, with neuronal loss also occurring in the neocortex. In some embodiments, medium spiny neurons (MSN) that contain enkephalin and that project to the external globus pallidum and/or MSN that contain substance P and project to the internal globus pallidum are affected. Other brain areas greatly affected in people with HD include the substantia nigra, cortical layers 3, 5, and 6, the CA1 region of the hippocampus, the angular gyrus in the parietal lobe, Purkinje cells of the cerebellum, lateral tuberal nuclei of the hypothalamus, and the centromedialparafascicular complex of the thalamus (Walker (2007) Lancet 369:218-228). Currently, there are no curative treatments for HD, but experimental approaches based on drugs, cell therapy, and gene therapy are under investigation.

RGN systems, polynucleotides encoding components of the RGN systems, RNP complexes, vectors, or compositions comprising any of these are useful for modifying a mutant HTT allele in vivo in HD patient cells. In some embodiments, modifying a mutant HTT allele includes modifying a target sequence in exon 50 of an HTT gene. For example, a APG07433.1 RGN is used with an appropriate guide RNA selected from SEQ ID NOs: 27 and 28 to modify a target sequence in exon 50 of a mutant HTT allele. As another example, a APG05586 RGN is used with an appropriate guide RNA selected from any of SEQ ID NOs: 25 and 26 to modify a target sequence in exon 50 of a mutant HTT allele. As yet another example, a APG01604 RGN is used with an appropriate guide RNA having the nucleotide sequence set forth as SEQ ID NO: 29 to modify a target sequence in exon 50 of a mutant HTT allele.

Modifying a target sequence in a mutant HTT allele includes cleaving a mutant HTT allele in vivo in HD patient cells. In some embodiments, the cleaving is in exon 50 of a mutant HTT allele. In some embodiments, after cleavage of the target sequence, non-homologous end joining (NHEJ) occurs, leading to insertions and/or deletions (indels) of nucleotides at the cleavage site and disruption of the mutant HTT coding sequence, leading to a reduction in the level of mutant HTT mRNA and/or protein. In some embodiments, cleavage of the mutHTT allele leads to the introduction of premature stop codons, leading to a reduction in mutHTT protein levels.

As used herein, the term “subject” refers to any individual for whom diagnosis, treatment or therapy is desired. In some embodiments, the subject is an animal. In some embodiments, the subject is a mammal. In some embodiments, the subject is a human being.

The methods of treating HD in the present disclosure take advantage of SNPs that occur in human genomes. SNP alleles have been identified that generate PAM sites that are recognized by RGN systems and RNP complexes of the present disclosure. When a mutant HTT allele comprises a SNP allele that generates a PAM recognized by an RGN system of the present disclosure, these RGN systems can be used to cleave the mutant HTT allele, leading to a reduction in the level of mutant HTT mRNA and/or protein. In some embodiments, a mutant HTT allele comprises the PAM comprising the SNP allele. Therefore, a subject in need thereof that would be suitable for treatment with the described compositions (e.g., an RGN or a nucleic acid molecule encoding the RGN, and a guide RNA or a nucleic acid molecule encoding the guide RNA; RGN systems; or RNP complexes) can be assayed prior to treatment to determine whether the mutant HTT allele comprises a SNP allele that generates a PAM recognized by an RGN system described herein. In some embodiments, a subject in need thereof that would be suitable for treatment with the described RGN systems further has a wild-type HTT allele that does not comprise the SNP allele that generates the PAM and is therefore heterozygous for the SNP. Having a wild-type HTT allele that does not comprise the SNP allele that generates the PAM is desirable, so that the administered composition only cleaves the mutant HTT allele comprising the SNP allele that generates a PAM and does not cleave the wild-type HTT allele. Therefore, in some embodiments, the PAM is present only on the mutant HTT allele and not on the wild-type HTT allele and the treatment is considered allele-specific. The methods can comprise use of an RGN system, polynucleotide encoding components of the RGN system, a RNP complex, vector, or a composition comprising any of these, that is not capable of cleaving a wild type HTT allele. In some embodiments, an RGN system, polynucleotide encoding components of the RGN system, a RNP complex, vector, or a composition comprising any of these, that is not capable of cleaving a wild type HTT allele is not capable of cleaving a wild type HTT allele at all or cleaves at a negligible level, such that the level of wtHTT mRNA and/or wtHTT protein is insignificantly decreased where, for example, the wtHTT can maintain support of critical cellular and neural functions and/or no symptoms of Huntington's disease is present in an in vivo setting (i.e. in a subject heterozygous for the mutHTT allele and administered the RGN system).

Determination of whether the subject has a mutant HTT allele comprising a SNP allele that generates a PAM recognized by an RGN system described herein and/or whether the SNP is heterozygous can utilize sequencing, array-based hybridization, and/or PCR-based methods (e.g., long-range PCR) performed on a biological sample obtained from the subject. In some embodiments, the biological sample can include blood, cells, and/or cerebrospinal fluid. In some embodiments, a mutant HTT allele comprises the PAM comprising the SNP allele.

A composition administered to a subject in need thereof comprises an RGN that can recognize the PAM generated by (i.e. comprising) a SNP allele (for example, in exon 50 of the mutHTT allele), which can include the NNNNCC, NNRYA, NNGRR, and NNGG PAM sequences. The SNP that generates the PAM can be within exon 50 of a mutant HTT allele. In some embodiments, the SNP is a thymine at a position corresponding to position 151 of SEQ ID NO: 1 that generates the PAM sequence NNRYA. In some embodiments, the NNRYA PAM sequence comprises the SNP that is a thymine at a position corresponding to position 151 of SEQ ID NO: 1. In some embodiments, the NNRYA PAM sequence is recognized by an RGN comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 7. In some embodiments, the NNRYA PAM sequence is recognized by an RGN comprising an amino acid sequence of SEQ ID NO: 7. In some embodiments, the NNRYA PAM sequence is recognized by an RGN that binds to a guide RNA comprising a crRNA repeat having the nucleotide sequence of SEQ ID NO: 8 or 106 or a nucleotide sequence that differs from SEQ ID NO: 8 or 106 by 1 or 2 nucleotides. In some embodiments, the NNRYA PAM sequence is recognized by an RGN that binds to a guide RNA comprising a tracrRNA having a nucleotide sequence having at least 90%, 91%, 92%, 93%, 94%, 95%, 96%, 97%, 98%, 99%, or more sequence identity to SEQ ID NO: 9 or 107. In some embodiments, the NNRYA PAM sequence is recognized by an RGN that binds to a guide RNA comprising a crRNA repeat having the nucleotide sequence of SEQ ID NO: 8 or 106 and a tracrRNA having a nucleotide sequence of SEQ ID NO: 9 or 107. In some embodiments, the NNRYA PAM sequence is recognized by an RGN that binds to a guide RNA comprising a spacer having a nucleotide sequence having complementarity with a target sequence having the nucleotide sequence of SEQ ID NO: 75 or 76. In some embodiments, the spacer has the nucleotide sequence of SEQ ID NO: 80 or 81 or a nucleotide sequence that differs from SEQ ID NO: 80 or 81 by 1 or 2 nucleotides. In some embodiments, the guide RNA is a single guide RNA. In some embodiments, the single guide RNA has the nucleotide sequence of SEQ ID NO: 25 or 26.

The SNP that generates the PAM can be within exon 50 of a mutant HTT allele. In some embodiments, the SNP is a cytosine at a position corresponding to position 151 of SEQ ID NO: 2 that generates the PAM sequence NNNNCC, NNGRR, and NNGG. In some embodiments, the PAM sequence NNNNCC, NNGRR, and NNGG comprises the SNP that is a cytosine at a position corresponding to position 151 of SEQ ID NO: 2. In some embodiments, the NNNNCC PAM sequence is recognized by an RGN comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 3. In some embodiments, the NNNNCC PAM sequence is recognized by an RGN comprising an amino acid sequence of SEQ ID NO: 3. In some embodiments, the NNNNCC PAM sequence is recognized by an RGN that binds to a guide RNA comprising a crRNA repeat having the nucleotide sequence of SEQ ID NO: 4 or a nucleotide sequence that differs from SEQ ID NO: 4 by 1 or 2 nucleotides. In some embodiments, the NNNNCC PAM sequence is recognized by an RGN that binds to a guide RNA comprising a tracrRNA having a nucleotide sequence having at least 90%, 91%, 92%, 93%, 94%, 95%, 96%, 97%, 98%, 99%, or more sequence identity to SEQ ID NO: 5. In some embodiments, the NNNNCC PAM sequence is recognized by an RGN that binds to a guide RNA comprising a crRNA repeat having the nucleotide sequence of SEQ ID NO: 4 and a tracrRNA having a nucleotide sequence of SEQ ID NO: 5. In some embodiments, the NNNNCC PAM sequence is recognized by an RGN that binds to a guide RNA comprising a spacer having a nucleotide sequence having complementarity with a target sequence having the nucleotide sequence of SEQ ID NO: 77 or 78. In some embodiments, the spacer has the nucleotide sequence of SEQ ID NO: 82 or 83 or a nucleotide sequence that differs from SEQ ID NO: 82 or 83 by 1 or 2 nucleotides. In some embodiments, the guide RNA is a single guide RNA. In some embodiments, the single guide RNA has the nucleotide sequence of SEQ ID NO: 27 or 28.

In some embodiments, the NNGRR PAM sequences is recognized by an RGN comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 11. In some embodiments, the NNGRR PAM sequence is recognized by an RGN comprising an amino acid sequence of SEQ ID NO: 11. In some embodiments, the NNGRR PAM sequence is recognized by an RGN that binds to a guide RNA comprising a crRNA repeat having the nucleotide sequence of SEQ ID NO: 12 or a nucleotide sequence that differs from SEQ ID NO: 12 by 1 or 2 nucleotides. In some embodiments, the NNGRR PAM sequence is recognized by an RGN that binds to a guide RNA comprising a tracrRNA having a nucleotide sequence having at least 90%, 91%, 92%, 93%, 94%, 95%, 96%, 97%, 98%, 99%, or more sequence identity to SEQ ID NO: 13 or 120. In some embodiments, the NNGRR PAM sequence is recognized by an RGN that binds to a guide RNA comprising a crRNA repeat having the nucleotide sequence of SEQ ID NO: 12 and a tracrRNA having a nucleotide sequence of SEQ ID NO: 13 or 120. In some embodiments, the NNGRR PAM sequence is recognized by an RGN that binds to a guide RNA comprising a spacer having a nucleotide sequence having complementarity with a target sequence having the nucleotide sequence of SEQ ID NO: 79. In some embodiments, the spacer has the nucleotide sequence of SEQ ID NO: 84 or a nucleotide sequence that differs from SEQ ID NO: 84 by 1 or 2 nucleotides. In some embodiments, the guide RNA is a single guide RNA. In some embodiments, the single guide RNA has the nucleotide sequence of SEQ ID NO: 29.

In some embodiments, the NNGG PAM sequences is recognized by an RGN comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 15. In some embodiments, the NNGG PAM sequence is recognized by an RGN comprising an amino acid sequence of SEQ ID NO: 15. In some embodiments, the NNGG PAM sequence is recognized by an RGN that binds to a guide RNA comprising a crRNA repeat having the nucleotide sequence of SEQ ID NO: 16 or a nucleotide sequence that differs from SEQ ID NO: 16 by 1 or 2 nucleotides. In some embodiments, the NNGG PAM sequence is recognized by an RGN that binds to a guide RNA comprising a tracrRNA having a nucleotide sequence having at least 90%, 91%, 92%, 93%, 94%, 95%, 96%, 97%, 98%, 99%, or more sequence identity to SEQ ID NO: 17. In some embodiments, the NNGG PAM sequence is recognized by an RGN that binds to a guide RNA comprising a crRNA repeat having the nucleotide sequence of SEQ ID NO: 16 and a tracrRNA having a nucleotide sequence of SEQ ID NO: 17. In some embodiments, the guide RNA is a single guide RNA.

As used herein, “treatment” or “treating” refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease or biomarkers of disease (e.g., neuronal cell phenotype changes), even though the disease, condition, or symptom may not have yet been manifested. In some embodiments, a subject at risk of developing HD is one containing a mutant HTT allele as defined herein. In some embodiments, the subject at risk of developing HD is defined as one having a mutant HTT allele comprising more than 35 CAG repeats. In some embodiments, the subject at risk of developing HD is defined as one having a mutant HTT allele comprising at least 40 CAG repeats. In some embodiments, the subject at risk of developing HD is defined as one having a mutant HTT allele comprising more than 56 CAG repeats. In some embodiments, treatment may be administered after one or more symptoms have developed and/or after a disease has been diagnosed. In some embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their prevention or recurrence. In some embodiments, the number of CAG repeats influences the decision of when to treat the subject. In some embodiments, subjects having a mutant HTT allele comprising more than 56 CAG repeats is treated as an adolescent or young adult before the onset of any HD symptoms or presentation of physiological biomarkers.

In some embodiments, the presently disclosed compositions and methods are used to ameliorate (i.e., reduce) or delay the onset of one or more symptoms of Huntington's disease in a subject in need thereof. A symptom may be reduced by about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% as compared to a control value or control subject (e.g., one that has not been administered the therapeutic agent). The onset of one or more symptoms of Huntington's disease may be delayed by about 6 months, 1 year, 2 years, 3 years, 4 years, 5 years, 6 years, 7 years, 8 years, 9 years, 10 years, 12 years, 15 years, 20 years, 25 years, 30 years or more as compared to a control value or control subject (e.g., one that has not been administered the therapeutic agent). In some embodiments, the presently disclosed compositions and methods are able to prevent the occurrence of one or more symptoms of Huntington's disease from developing in a subject.

In some embodiments, the presently disclosed compositions and methods are used to ameliorate (i.e., reduce) or delay the onset of one or more biomarkers of Huntington's disease in a subject in need thereof. A biomarker may be reduced by about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% as compared to a control value or control subject (e.g., one that has not been administered the therapeutic agent). The onset of one or more biomarker of Huntington's disease may be delayed by about 6 months, 1 year, 2 years, 3 years, 4 years, 5 years, 6 years, 7 years, 8 years, 9 years, 10 years, 12 years, 15 years, 20 years, 25 years, 30 years or more as compared to a control value or control subject (e.g., one that has not been administered the therapeutic agent). In some embodiments, the presently disclosed compositions and methods are able to prevent the development of one or more biomarkers of Huntington's disease from developing in a subject.

A subject at risk for developing HD or who is afflicted by HD may be identified in various ways, including cognitive assessments and/or neurological or neuropsychiatric examinations, motor tests, sensory tests, psychiatric evaluations, brain imaging, family history and/or genetic testing. A subject in need thereof may have symptoms of HD, be diagnosed with HD, and/or may be asymptomatic for HD.

The compositions of the present disclosure (e.g., comprising an RGN or a nucleic acid molecule encoding the RGN, and a guide RNA or a nucleic acid molecule encoding the guide RNA; RGN systems; RNP complexes; or vectors) may be administered to a subject who is staged using the Huntington's Disease Integrated Staging System (HD-ISS) (Tabrizi et al. Lancet Neurol 2022; 21: 632-644, the contents of which are herein incorporated by reference in their entirety). In this system, Stage 0 HD subjects have 40 CAG repeats. Stage 1 HD subjects have 40 CAG repeats and have biomarkers of pathogenesis (e.g., putamen volume and/or caudate volume). Stage 2 HD subjects have 40 CAG repeats, biomarkers of pathogenesis, and signs or symptoms (e.g., as measured by Total Motor Score (TMS) and/or Symbol Digit Modalitites Test (SDMT)). Stage 3 HD subjects have 40 CAG repeats, biomarkers of pathogenesis, signs or symptoms, and functional change (e.g., as measured by the Independence Scale and/or the Total Functional Capacity (TFC)).

The compositions of the present disclosure (e.g., comprising an RGN or a nucleic acid molecule encoding the RGN, and a guide RNA or a nucleic acid molecule encoding the guide RNA; RGN systems; RNP complexes; or vectors) may be administered to a subject who is diagnosed using the Prognostic Index for Huntington's Disease, or a derivative thereof (Long J D et al., Movement Disorders, 2017, 32(2), 256-263, the contents of which are herein incorporated by reference in their entirety). This prognostic index uses four components to predict probability of motor diagnosis: (1) total motor score (TMS) from the Unified Huntington's Disease Rating Scale (UHDRS), (2) Symbol Digit Modality Test (SDMT), (3) base-line age, and (4) CAG expansion. In some embodiments, the prognostic index for HD is calculated with the following formula: PI_{HD}=51×TMS+(−34)×SDMT+7×Age×(CAG-34), wherein larger values for PI_{HD} indicate greater risk of diagnosis or onset of symptoms. In some embodiments, the prognostic index for HD is calculated with the following normalized formula that gives standard deviation units to be interpreted in the context of 50% 10-year survival: PIN_{HD}=(PI_{HD}−883)/1044, wherein PIN_{HD}<0 indicates greater than 50% 10-year survival, and PIN_{HD}>0 suggests less than 50% 10-year survival. In some embodiments, the prognostic index may be used to identify subjects who will develop symptoms of HD within several years, but that do not yet have clinically diagnosable symptoms. Further, these asymptomatic patients may be selected for and receive treatment using the compositions of the present disclosure during the asymptomatic period.

The compositions of the present disclosure (e.g., comprising an RGN or a nucleic acid molecule encoding the RGN, and a guide RNA or a nucleic acid molecule encoding the guide RNA; RGN systems; RNP complexes; or vectors) may be administered to a subject who has undergone biomarker assessment. Potential biomarkers in blood for HD include, but are not limited to, 8-hydroxy-2-deoxyguanosine (8-OhdG) oxidative stress marker, metabolic markers (e.g., creatine kinase, branched-chain amino acids), cholesterol metabolites (e.g., 24-OH cholesterol), immune and inflammatory proteins (e.g., clusterin, complement components, interleukins 6 and 8), gene expression changes (e.g., transcriptomic markers), endocrine markers (e.g., cortisol, ghrelin and leptin), brain-derived neurotrophic factor (BDNF), and adenosine 2A receptors. Potential biomarkers for brain imaging for HD include, but are not limited to, striatal volume, putamen volume, caudate volume, subcortical white-matter volume, cortical thickness, whole brain volume, and ventricular volumes. Brain imaging can be performed using functional imaging (e.g., functional MRI), positron emission tomography (PET) (e.g., with fluorodeoxyglucose), and magnetic resonance spectroscopy (e.g., lactate). Potential biomarkers for quantitative clinical tools for HD include, but are not limited to, quantitative motor assessments, motor physiological assessments (e.g., transcranial magnetic stimulation), and quantitative eye movement measurements. Non-limiting examples of quantitative clinical biomarker assessments include tongue force variability, metronome-guided tapping, grip force, oculomotor assessments, and cognitive tests. Non-limiting examples of multicenter observational studies include PREDICT-HD and TRACK-HD. In some embodiments, the biomarker for HD is levels of wild type huntingtin (HTT) mRNA and/or protein. In some embodiments, the biomarker for HD is levels of mutant huntingtin (HTT) mRNA and/or protein. In some embodiments, the biomarker for HD is levels of neurofilament light chain (NFL) protein.

The compositions of the present disclosure (e.g., comprising an RGN or a nucleic acid molecule encoding the RGN, and a guide RNA or a nucleic acid molecule encoding the guide RNA; RGN systems; RNP complexes; or vectors) may be administered to a subject who is asymptomatic for HD. A subject may be asymptomatic but may have undergone predictive genetic testing or biomarker assessment to determine if they are at risk for HD and/or a subject may have a family member (e.g., mother, father, brother, sister, aunt, uncle, grandparent) who has been diagnosed with HD. In some embodiments, a subject who is asymptomatic for HD has a mutant HTT allele comprising 27-35 CAG repeats (e.g., 27, 28, 29, 30, 31, 32, 33, 34, and 35 CAG repeats).

The compositions of the present disclosure (e.g., comprising an RGN or a nucleic acid molecule encoding the RGN, and a guide RNA or a nucleic acid molecule encoding the guide RNA; RGN systems; RNP complexes; or vectors) may be administered to a subject who is in the early stages of HD. In the early stage a subject can have subtle changes in coordination, some chorea, changes in mood such as irritability and depression, problem solving difficulties, and/or a reduction in the ability of the subject to function in their normal day to day life.

The compositions of the present disclosure (e.g., comprising an RGN or a nucleic acid molecule encoding the RGN, and a guide RNA or a nucleic acid molecule encoding the guide RNA; RGN systems; RNP complexes; or vectors) may be administered to a subject who is in the middle stages of HD. In the middle stage a subject has an increase in the movement disorder, diminished speech, difficulty swallowing, and ordinary activities will become harder to do. At this stage a subject may have occupational and physical therapists to help maintain control of voluntary movements and the subject may have a speech language pathologist.

The compositions of the present disclosure (e.g., comprising an RGN or a nucleic acid molecule encoding the RGN, and a guide RNA or a nucleic acid molecule encoding the guide RNA; RGN systems; RNP complexes; or vectors) may be administered to a subject who is in the late stages of HD. In the late stage, a subject with HD is almost completely or completely dependent on others for care as the subject can no longer walk and is unable to speak. A subject can generally still comprehend language and is aware of family and friends but choking is a major concern.

The compositions of the present disclosure (e.g., comprising an RGN or a nucleic acid molecule encoding the RGN, and a guide RNA or a nucleic acid molecule encoding the guide RNA; RGN systems; RNP complexes; or vectors) may be administered to a subject who has juvenile HD which is the onset of HD before the age of 21 years, or is susceptible to developing juvenile HD, such as those subjects having at least 56 CAG repeats in exon 1 of the HTT gene (e.g., 56, 57, 58, 59, 60, 61, 62, 63, 64, 65, 66, 67, 68, 69, 70, 71, 72, 73, 74, 75 or more than 75 CAG repeats). In some of these embodiments, the subject is administered the presently disclosed compositions before the age of 21 years or in some embodiments, before the age of 18 years.

The compositions of the present disclosure (e.g., comprising an RGN or a nucleic acid molecule encoding the RGN, and a guide RNA or a nucleic acid molecule encoding the guide RNA; RGN systems; RNP complexes; or vectors) may be administered to a subject who has fully penetrant HD where the mutant HTT allele has greater than 40 CAG repeats in exon 1 (e.g., 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more than 90 CAG repeats). In some embodiments, a subject in need thereof that can be treated with the compositions of the disclosure has a mutant HTT allele comprising at least 36 CAG repeats in exon 1 (e.g., 36, 37, 38, 39, 40, 41, 42, 43, 44, 45, 46, 47, 48, 49, 50, or more than 50 CAG repeats).

The compositions of the present disclosure (e.g., comprising an RGN or a nucleic acid molecule encoding the RGN, and a guide RNA or a nucleic acid molecule encoding the guide RNA; RGN systems; RNP complexes; or vectors) may be administered to a subject with HD who has incomplete penetrance where the mutant HTT allele has between 36 and 39 CAG repeats (e.g., 36, 37, 38, and 39 CAG repeats).

In some embodiments, a control reference or a healthy subject has an HTT allele with no more than 15-20 CAG trinucleotide repeats. In some embodiments, a control reference or a healthy subject has an HTT allele with no more than 26 CAG trinucleotide repeats.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, and the delivery system in which it is carried.

The efficacy of a treatment can be determined by the skilled clinician. However, a treatment is considered an “effective treatment,” if any one or all of the signs or symptoms of a disease or disorder are altered in a beneficial manner (e.g., decreased by at least 10%), or other clinically accepted symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art. Treatment includes: (1) inhibiting the disease, e.g., arresting, or slowing the progression of symptoms; or (2) relieving the disease, e.g., causing regression of symptoms; and (3) preventing or reducing the likelihood of the development of symptoms.

Symptoms of HD may include features attributed to central nervous system (CNS) degeneration such as, but are not limited to, chorea (involuntary movements), dystonia, bradykinesia incoordination, irritability and depression, problem solving difficulties, reduction in the ability of a person to function in their normal day to day life, diminished speech, and difficulty swallowing, as well as features not attributed to CNS degeneration such as, but not limited to, weight loss, muscle wasting, metabolic dysfunction and endocrine disturbances. In some embodiments, symptoms of HD include behavioral difficulties and symptoms such as, but not limited to, apathy or lack of initiative, dysphoria, irritability, agitation or anxiety, poor self-care, poor judgment, inflexibility, disinhibition, depression, suicidal ideation, euphoria, aggression, delusions, compulsions, hypersexuality, hallucinations, speech deterioration, slurred speech, difficulty swallowing, weight loss, cognitive dysfunction which impairs executive functions (e.g., organizing, planning, checking or adapting alternatives, and delays in the acquisition of new motor skills), unsteady gait, and chorea. In some embodiments, the survival of the subject is prolonged by treating any of the symptoms of HD described herein.

Compositions of the disclosure (e.g., comprising an RGN or a nucleic acid molecule encoding the RGN, and a guide RNA or a nucleic acid molecule encoding the guide RNA; RGN systems; RNP complexes; or vectors) can be considered efficacious in treating HD when motor functioning, cognitive functioning, emotional/behavioral functioning, functional limitations, and/or other assessments improve as compared to a control value, or approach or equal a control value. An improvement in motor function or a reduction in motor decline of a subject with HD can be measured by a standard evaluation system such as: the Unified Huntington's Disease Ratings Scale (UHDRS; Mov Disord. 1996 March; 11(2):136-142); the NIH Toolbox-Motor Function: Endurance (2-min. walk test), Locomotion (4-meter walk test) (Gershon et al. Lancet Neurol. 2010 February; 9(2):138-139); the Timed Up and Go (Rao et al. Gait Posture. 2009 April; 29(3):433-436); and/or the 10-Meter Walking Test (Rao et al. Spectrum of gait impairments in presymptomatic and symptomatic Huntington's disease. Mov Disord. 2008 Jun. 15; 23(8):1100-1107). An improvement in cognitive function or a reduction in cognitive decline can be measured by an evaluation system such as: Symbol Digit Modalities Test (Smith A. Symbol digit modalities test: Manual. Los Angeles: Western Psychological Services; 1982); Self-Paced Tapping (Rowe et al. Neuropsychology. 2010 July; 24(4):435-442); Speeded Tapping Test (Stout et al. Neuropsychology. 2011 January; 25(1):1-14); Stroop Color Naming (Stroop JR. Studies of interference in serial verbal reactions. J Exp Psychol. 1935; 18:643-662); and/or Stroop Word Reading (Stroop JR. Studies of interference in serial verbal reactions. J Exp Psychol. 1935; 18:643-662). An improvement in emotional/behavioral function or a reduction in emotional/behavioral decline of a subject with HD can be measured by a standard evaluation system such as: Problem Behaviors Assessment—Short (Craufurd et al. Neuropsychiatry Neuropsychol Behav Neurol. 2001 October-December; 14(4):219-226); Apathy Evaluation Scale (Marin et al. Psychiatry Res. 1991 August; 38(2):143-162); Columbia Suicide Severity Scale (Posner et al. Am J Psychiatry. 2011 December; 168(12):1266-1277); Hospital Anxiety and Depression Scale (Zigmond and Snaith. Acta Psychiatr Scand. 1983 June; 67(6):361-370); and/or Irritability Scale (Chatterjee et al. J Neuropsychiatry Clin Neurosci. 2005 Summer; 17(3):378-383). Functional assessments for a subject with HD can be measured by the UHDRS Functional Assessment Checklist, the UHDRS Independence Scale, and/or the UHDRS Total Functional Capacity.

In subjects classified as having or at risk of developing HD, a mutant HTT protein is produced and is toxic. Thus, compositions of the disclosure can be considered efficacious in treating HD when a mutant HTT protein level is reduced or eliminated. The mutant HTT protein may be reduced by about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in the CNS, a region of the CNS, or a specific cell of the CNS of a subject in need thereof, as compared to a control value or a control subject (e.g., a subject that has not been administered the therapeutic agent) or wild type HTT protein. The mutant HTT mRNA may be reduced by about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in the CNS, a region of the CNS, or a specific cell of the CNS of a subject in need thereof, as compared to a control value or control subject (e.g., a subject that has not been administered the therapeutic agent) or wild type HTT mRNA. In some embodiments, the reduction in mutHTT mRNA or mutHTT protein is observed by 4 weeks, 6 weeks, 8 weeks, 10 weeks, 12 weeks, or later after administration of the therapeutic agent. The decrease in mutHTT protein can be observed in at least 30%, 40%, 50%, or more than 50% of the striatal cells in the subject to which the therapeutic agent has been administered.

In some embodiments, a composition of the disclosure (e.g., comprising an RGN or a nucleic acid molecule encoding the RGN, and a guide RNA or a nucleic acid molecule encoding the guide RNA; RGN systems; RNP complexes; or vectors), when administered to a subject in need thereof, may reduce mutant HTT protein and/or mRNA in: the striatum; the putamen; the caudate; the cortex; the motor cortex; the somatosensory cortex; the temporal cortex; the substantia nigra; the hippocampus; the parietal lobe; the angular gyrus; the midbrain; the forebrain; the cerebellum; the hypothalamus; and/or the thalamus of the CNS. In some embodiments, a composition of the disclosure (e.g., an RGN or a nucleic acid molecule encoding the RGN, and a guide RNA or a nucleic acid molecule encoding the guide RNA; RGN systems; or RNP complexes), when administered to a subject in need thereof, may reduce mutant HTT protein and/or mRNA in: neurons, neural progenitor cells, forebrain neuron progenitor cells, striatal neurons, medium spiny neurons, cortical neurons, glial cells (e.g., microglia, astrocytes, oligodendrocytes), Purkinje cells of the cerebellum, lateral tuberal nuclei of the hypothalamus, and/or the centromedialparafascicular complex of the thalamus.

In some embodiments, a level of mutant HTT protein and/or mutant HTT mRNA is increased in subjects classified as having or at risk of developing HD relative to a control reference. Therefore, a decrease in the level of or elimination of mutant HTT protein and/or mutant HTT mRNA provides an indication of a favorable treatment response, whereas an unchanged or an increasing level of mutant HTT protein and/or mutant HTT mRNA provides an indication of unfavorable or at least non-optimal treatment response. A level of mutant HTT protein and/or mutant HTT mRNA can be measured in a biological sample obtained from a subject including, but not limited to, blood, cerebrospinal fluid, and cells. Mutant HTT protein levels can be measured by assays including immunoassays that use antibodies that can distinguish between wild-type and mutant HTT protein (e.g., Western blot, ELISA, single-molecule counting immunoassay, immunoprecipitation assay combined with flow cytometry, time-resolved fluorescence energy transfer (TR-FRET)). Mutant HTT mRNA levels can be measured by, e.g., RT-qPCR or array-based methods. In some embodiments, a control reference includes a biological sample from a healthy subject or population of healthy subjects having an HTT gene comprising no more than 26 CAG trinucleotide repeats. Mutant HTT levels as compared to wild-type HTT levels can be measured using the JESS capillary western blot method as described in the examples herein.

In some embodiments, assessing prophylactic or therapeutic treatment can include measuring a baseline value of a measurable biomarker or disease parameter in a subject classified as having or at risk of developing HD before administering a dosage of a composition described herein, and comparing this with a value for the same measurable biomarker or parameter after a course of treatment.

In some embodiments, a control value (i.e., a mean and standard deviation) of the measurable biomarker or parameter is determined for a control population. In some embodiments, the individuals in the control population have not received prior treatment and do not have HD, nor are at risk of developing HD. In some embodiments, the individuals in the control population are prophylactically treated subjects who remain free of HD symptoms, or are therapeutically treated subjects who show amelioration of HD symptoms. In such cases, if the value of the measurable biomarker or clinical parameter approaches the control value, then treatment is considered efficacious. In some embodiments, the individuals in the control population have not received prior treatment and have been diagnosed with HD. In such cases, if the value of the measurable biomarker or clinical parameter approaches the control value, then treatment is considered inefficacious. In all of these cases, a significant difference relative to the control value (i.e., more than a standard deviation) is an indicator for efficacy or non-efficacy.

The term “administering” refers to the placement of an active ingredient into a subject in need thereof, by a method or route that results in at least partial localization of the introduced active ingredient at a desired site, such that a desired effect(s) is produced. In embodiments, the disclosure provides methods comprising delivering any of the RGN systems, nucleic acid molecules, ribonucleoprotein complexes, vectors, pharmaceutical compositions, and/or gRNAs described herein. In some embodiments, the disclosure further provides cells produced by such methods, and organisms (such as animals or plants) comprising or produced from such cells. In some embodiments, a RGN polypeptide and/or nucleic acid molecules as described herein in combination with (and optionally complexed with) a guide sequence is delivered to a cell in vivo.

In some embodiments, the administering comprises administering by viral delivery. Viral vectors comprising a nucleic acid encoding the nucleases, RGN polypeptides, ribonucleoprotein complexes, or vectors disclosed herein may be administered directly to patients (i.e., in vivo). Conventional viral based systems may include, without limitation, retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Lentiviral vectors are retroviral vectors that are able to transduce or infect non-dividing cells and typically produce high viral titers. In applications where transient expression is preferred, adenoviral based systems may be used. Adenoviral based vectors are capable of very high transduction efficiency in many cell types and do not require cell division.

The adeno-associated virus (AAV) is a helper-dependent DNA parvovirus belonging to the genus Dependovirus. AAV-based vectors are attractive vectors for gene therapy in large part because this virus is apparently non-pathogenic. AAV can transduce dividing and non-diving cells (including efficient transduction of neurons), has diminished proinflammatory and immune responses in humans, is not able to autonomously replicate without a helper virus, and can have long-term gene expression. Over 10 recombinant AAV serotypes (rAAV) have been engineered into vectors. In some embodiments, an AAV vector useful for the described methods include AAV5.

Delivery of the compositions in accordance with the present disclosure to cells in vivo can comprises a rate of delivery defined by [VG/hour=mL/hour*VG/mL] wherein VG is viral genomes, VG/mL is composition concentration, and mL/hour is rate of prolonged delivery. In some embodiments, delivery of compositions in accordance with the present disclosure to cells in vivo may comprise a total concentration per subject between about 1×10⁶ VG and about 1×10¹⁶ VG.

In some embodiments, the administering comprises administering by other non-viral delivery of nucleic acids. Exemplary non-viral delivery methods, without limitation, include RNP complexes, lipofection, nucleofection, microinjection, biolistics, virosomes, liposomes, immunoliposomes, polycation or lipidmucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO1991/17424; WO 1991/16024. Delivery can be to cells or target tissues in vivo.

Suitable routes of administering the pharmaceutical compositions described herein include, without limitation: intrastriatal (intra-putamen or intra-caudate), intrathecal, intraparenchymal, intracerebral, intracerebroventricular, intrathalamic, intra-cisterna magna, subpial, intravenous, and intravascular administration. In some embodiments, administering the pharmaceutical compositions is by intrastriatal injection, intrathecal administration, intraparenchymal injection, putamen fusion, caudate fusion, intracerebroventricular, intrathalamic, subpial administration, transmucosal administration, or nasal administration. In some embodiments, administration of the pharmaceutical compositions described herein into one region of the brain (e.g. the striatum) allows the pharmaceutical compositions to migrate to a nearby region (e.g., the cortex). In some embodiments, the pharmaceutical composition is administered bilaterally. In some embodiments, the pharmaceutical composition is administered unilaterally.

In some embodiments, the pharmaceutical composition described herein is administered to a subject by injection, by means of a catheter, or by means of an implant, the implant being of a porous, non-porous, or gelatinous material, including a membrane, such as a sialastic membrane, or a fiber. In some embodiments, the pharmaceutical composition described herein is administered to a subject by means of a drug delivery system, such as a catheter system for direct delivery of therapeutics to the brain. In some embodiments, the drug delivery system is a Neuroinfuse™ drug delivery system (Renishaw Neuro Solutions Limited). In some embodiments, the drug delivery system performs multiple injections at the same time, such as 2, 3, 4, 5, 6, 7, or 8 injections. In some embodiments, the pharmaceutical composition described herein is administered to one hemisphere of the brain. In some embodiments, the pharmaceutical composition described herein is administered to both hemispheres of the brain. In some embodiments, the pharmaceutical composition is formulated for delivery to a subject, e.g., for gene editing.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

In some embodiments, the pharmaceutical composition can be contained within a lipid particle or vesicle, such as a liposome or microcrystal, which is also suitable for parenteral administration.

The desired composition dosage may be delivered using multiple administrations (e.g., two, three, four, five, six, seven, eight, nine, ten, eleven, twelve, thirteen, fourteen, or more administrations). When multiple administrations are employed, split dosing regimens may be used. A “split dose” is the division of single unit dose or total daily dose into two or more doses, e.g., two or more administrations of the single unit dose. A “single unit dose” is a dose of any therapeutic composition (e.g., comprising an RGN or a nucleic acid molecule encoding the RGN, and a guide RNA or a nucleic acid molecule encoding the guide RNA; RGN systems; or RNP complexes) administered in one dose/at one time/single route/single point of contact, i.e., single administration event. A “total daily dose” is an amount given or prescribed in a 24 hour period. It may be administered as a single unit dose.

Although the descriptions of pharmaceutical compositions provided herein are principally directed to pharmaceutical compositions which are suitable for administration to humans, it will be understood by the skilled artisan that such compositions are generally suitable for administration to animals or organisms of all sorts.

### XII. Capillary Electrophoresis Immunoassay

Levels of mutHTT and wtHTT protein can be detected and quantified using a capillary electrophoresis immunoassay, such as the assay described in Example 9 herein. Thus, provided herein are methods for detecting mutant huntingtin (mutHTT) protein and wild type HTT (wtHTT) protein in a sample, wherein the method comprises: a) applying a sample (e.g., biological sample comprising proteins) that has been denatured (e.g., heat denatured, denatured with sodium dodecyl sulfate, or both) to a capillary comprising a sieving medium (e.g., a hydrophilic polymer matrix) that can comprise SDS; b) applying a voltage differential to the capillary to separate proteins within the sample by molecular weight via electrophoresis; c) immobilizing the separated proteins within the capillary; d) applying to the capillary a first antibody or fragment thereof capable of binding to both mutHTT and wtHTT; e) applying to the capillary a second antibody or fragment thereof capable of binding to the first antibody, wherein the second antibody comprises a detectable label (e.g., chemiluminescent label or fluorescent label); and f) detecting the detectable label. The method can further comprise quantitating the amounts of mutHTT and wtHTT proteins within the sample by comparing to a standard curve (i.e., increasing known amounts of a mutHTT or wtHTT standard). In some embodiments, the anti-HTT antibody does not detect the HTT protein within the polyQ region. The anti-HTT antibody can detect the amino terminus of the HTT protein, such as for example, amino acid residues 7-13. The immobilizing step can be through the use of capillaries that comprise one or more reactive moieties capable of immobilizing the separate proteins and the immobilizing includes activating the reactive moieties. The activating step can comprise photoactivation, chemical activation or thermal activation of the activated reactive moieties. Non-limiting examples of such assays are described in U.S. Pat. Nos. 7,935,489; 7,846,676; 9,304,133; 7,935,308; 7,935,479; 9,400,277; 8,940,232; 8,021,611; 8,945,361; 9,766,206; 11,143,650; 11,237,164; 11,535,900; U.S. patent application Ser. Nos. 15/707,600; 16/423,787; 17/589,350; each of which is incorporated by reference in its entirety.

The article “a” and “an” are used herein to refer to one or more than one (i.e., to at least one) of the grammatical object of the article. By way of example, “a polypeptide” means one or more polypeptides.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

Non-limiting embodiments include:

1. An RNA-guided nuclease (RGN) system comprising:
a) a guide RNA comprising a spacer and a backbone, wherein said spacer has the nucleotide sequence of SEQ ID NO: 80 or 81 or a nucleotide sequence that differs from SEQ ID NO: 80 or 81 by 1 or 2 nucleotides, or a nucleic acid molecule encoding the guide RNA; and
b) an RGN polypeptide having an amino acid sequence having at least 90% sequence identity to SEQ ID NO: 7 or a nucleic acid molecule encoding the RGN polypeptide.

2. The RGN system of embodiment 1, wherein said guide RNA comprises a spacer having a nucleotide sequence that differs from SEQ ID NO: 80 or 81 by 2 nucleotides.

3. The RGN system of embodiment 1, wherein said guide RNA comprises a spacer having a nucleotide sequence that differs from SEQ ID NO: 80 or 81 by 1 nucleotide.

4. The RGN system of embodiment 1, wherein said guide RNA comprises a spacer having the nucleotide sequence of SEQ ID NO: 80 or 81.

5. The RGN system of any one of embodiments 1-4, wherein said guide RNA binds to a target sequence in a mutant huntingtin (mutHTT) allele.

6. The RGN system of embodiment 5, wherein said RGN system is capable of binding and cleaving a target sequence in said mutHTT allele, and wherein the guide RNA is capable of forming a complex with the RGN polypeptide and directing the complex to the target sequence for binding and cleaving.

7. The RGN system of embodiment 5 or 6, wherein said target sequence has the nucleotide sequence of SEQ ID NO: 75 or 76.

8. The RGN system of any one of embodiments 1-7, wherein said RGN system is capable of recognizing a protospacer adjacent motif (PAM) having the sequence of NNRYA created by a single nucleotide polymorphism (SNP) in exon 50 of the HTT gene.

9. The RGN system of any one of embodiments 1-8, wherein said RGN polypeptide comprises a PAM-interacting domain that binds a protospacer adjacent motif (PAM) having the sequence of NNRYA created by a single nucleotide polymorphism (SNP) in exon 50 of the HTT gene.

10. The RGN system of embodiment 9, wherein said PAM-interacting domain comprises an amino acid sequence having at least 95% sequence identity to SEQ ID NO: 134.

11. The RGN system of embodiment 9 or 10, wherein said PAM-interacting domain comprises the amino acid sequence set forth as SEQ ID NO: 134.

12. The RGN system of any one of embodiments 1-11, wherein said RGN polypeptide comprises at least one nuclease domain comprising an amino acid sequence having at least 95% sequence identity to any one of SEQ ID NOs: 147, 148, 149, and 150.

13. The RGN system of any one of embodiments 1-12, wherein said RGN system is not capable of cleaving a wild type HTT allele.

14. The RGN system of any one of embodiments 1-13, wherein said RGN polypeptide has an amino acid sequence having at least 95% sequence identity to SEQ ID NO: 7.

15. The RGN system of any one of embodiments 1-14, wherein said RGN polypeptide has the amino acid sequence of SEQ ID NO: 7.

16. The RGN system of any one of embodiments 1-15, wherein said RGN polypeptide further comprises at least one nuclear localization signal.

17. The RGN system of embodiment 16, wherein said at least one nuclear localization signal comprises an SV40 nuclear localization signal.

18. The RGN system of embodiment 17, wherein said SV40 nuclear localization signal has the sequence set forth as SEQ ID NO: 86.

19. The RGN system of embodiment 16, wherein said at least one nuclear localization signal comprises a c-Myc nuclear localization signal.

20. The RGN system of embodiment 19, wherein said c-Myc nuclear localization signal has the sequence set forth as SEQ ID NO: 125.

21. The RGN system of any one of embodiments 16-20, wherein a NLS linker protein connects said RGN polypeptide and said at least one nuclear localization signal.

22. The RGN system of embodiment 21, wherein said NLS linker protein has the sequence set forth as SEQ ID NO: 127.

23. The RGN system of any one of embodiments 1-22, wherein the backbone of the guide RNA is 66 to 90 nucleotides in length.

24. The RGN system of any one of embodiments 1-23, wherein the backbone of the guide RNA comprises a nucleotide sequence having at least 80% sequence identity to SEQ ID NO: 140 or 141.

25. The RGN system of any one of embodiments 1-24, wherein said nucleic acid molecule encoding said RGN polypeptide is operably linked to an RNA polymerase II promoter.

26. The RGN system of embodiment 25, wherein said RNA polymerase II promoter is a constitutive promoter.

27. The RGN system of embodiment 26, wherein said constitutive promoter is selected from the group consisting of: a cytomegalovirus (CMV) promoter, a truncated CMV promoter, an elongation factor 1α short (EFS) promoter, and a JeT promoter.

28. The RGN system of embodiment 27, wherein said constitutive promoter is a JeT promoter.

29. The RGN system of embodiment 28, wherein said JeT promoter has the nucleotide sequence set forth as SEQ ID NO: 92.

30. The RGN system of embodiment 25, wherein said RNA polymerase II promoter is a tissue-specific promoter.

31. The RGN system of embodiment 30, wherein said tissue-specific promoter is a brain or neuron specific promoter.

32. The RGN system of embodiment 31, wherein said brain or neuron specific promoter is selected from the group consisting of: a human synapsin I (Syn) promoter, a 67 kDa glutamic acid decarboxylase (GAD67) promoter, a 65 kDa glutamic acid decarboxylase (GAD65) promoter, a homeobox Dlx5/6 promoter, a preprotachykinin 1 (Tac1) promoter, a neuron-specific enolase (NSE) promoter, a dopaminergic receptor 1 (Drd1a) promoter, a dopaminergic receptor 2 (DRD2) promoter, and glial fibrillary acidic protein (GFAP) promoter.

33. The RGN system of embodiment 32, wherein said neuron specific promoter is a Syn promoter.

34. The RGN system of embodiment 33, wherein said Syn promoter has the nucleotide sequence set forth as SEQ ID NO: 93.

35. The RGN system of any one of embodiments 1-34, wherein said nucleic acid molecule encoding said RGN polypeptide comprises a polyadenylation (polyA) tail.

36. The RGN system of embodiment 35, wherein said polyA tail is a SV40 polyA tail or a bovine growth hormone (bGH) polyA tail.

37. The RGN system of embodiment 36, wherein said SV40 polyA tail has the nucleotide sequence set forth as SEQ ID NO: 94.

38. The RGN system of embodiment 36, wherein said bGH polyA tail has the sequence set forth as SEQ ID NO: 95.

39. The RGN system of any one of embodiments 1-38, wherein said guide RNA comprises a crRNA repeat having the nucleotide sequence of SEQ ID NO: 8 or 106 or a nucleotide sequence that differs from SEQ ID NO: 8 or 106 by 1 or 2 nucleotides and a tracrRNA having a nucleotide sequence having at least 90% sequence identity to SEQ ID NO: 9 or 107.

40. The RGN system of any one of embodiments 1-38, wherein said guide RNA comprises a crRNA repeat having a nucleotide sequence that differs from SEQ ID NO: 8 or 106 by 1 nucleotide and a tracrRNA having a nucleotide sequence having at least 95% sequence identity to SEQ ID NO: 9 or 107.

41. The RGN system of any one of embodiments 1-38, wherein said guide RNA comprises a crRNA repeat having the nucleotide sequence of SEQ ID NO: 8 or 106 and a tracrRNA having the nucleotide sequence of SEQ ID NO: 9 or 107.

42. The RGN system of any one of embodiments 1-41, wherein said guide RNA is a single guide RNA.

43. The RGN system of embodiment 42, wherein said single guide RNA has the nucleotide sequence of SEQ ID NO: 25 or 26.

44. A ribonucleoprotein (RNP) complex comprising the RGN polypeptide and the guide RNA of said RGN system of any one of embodiments 1-43.

45. A nucleic acid molecule comprising or encoding a guide RNA that comprises a spacer having the nucleotide sequence of SEQ ID NO: 80 or 81 or a nucleotide sequence that differs from SEQ ID NO: 80 or 81 by 1 or 2 nucleotides.

46. The nucleic acid molecule of embodiment 45, wherein said guide RNA comprises a spacer having a nucleotide sequence that differs from SEQ ID NO: 80 or 81 by 2 nucleotides.

47. The nucleic acid molecule of embodiment 45, wherein said guide RNA comprises a spacer having a nucleotide sequence that differs from SEQ ID NO: 80 or 81 by 1 nucleotide.

48. The nucleic acid molecule of embodiment 45, wherein said guide RNA comprises a spacer having the nucleotide sequence of SEQ ID NO: 80 or 81.

49. The nucleic acid molecule of any one of embodiments 45-48, wherein said guide RNA binds to a target sequence in a mutant huntingtin (mutHTT) allele.

50. The nucleic acid molecule of embodiment 49, wherein said target sequence has the nucleotide sequence of SEQ ID NO: 75 or 76.

51. The nucleic acid molecule of any one of embodiments 45-50, wherein said guide RNA binds to an RNA-guided nuclease (RGN) polypeptide having an amino acid sequence having at least 90% sequence identity to SEQ ID NO: 7.

52. A nucleic acid molecule comprising or encoding a guide RNA that binds to a target sequence in a mutant huntingtin (mutHTT) allele, wherein said target sequence has the nucleotide sequence of SEQ ID NO: 75 or 76 and binds to an RNA-guided nuclease (RGN) polypeptide having an amino acid sequence having at least 90% sequence identity to SEQ ID NO: 7.

53. The nucleic acid molecule of embodiment 52, wherein said guide RNA comprises a spacer having the nucleotide sequence of SEQ ID NO: 80 or 81 or a nucleotide sequence that differs from SEQ ID NO: 80 or 81 by 1 or 2 nucleotides.

54. The nucleic acid molecule of embodiment 53, wherein said guide RNA comprises a spacer having a nucleotide sequence that differs from SEQ ID NO: 80 or 81 by 2 nucleotides.

55. The nucleic acid molecule of embodiment 53, wherein said guide RNA comprises a spacer having a nucleotide sequence that differs from SEQ ID NO: 80 or 81 by 1 nucleotide.

56. The nucleic acid molecule of embodiment 53, wherein said guide RNA comprises a spacer having the nucleotide sequence of SEQ ID NO: 80 or 81.

57. The nucleic acid molecule of any one of embodiments 51-56, wherein said RGN polypeptide has an amino acid sequence having at least 95% sequence identity to SEQ ID NO: 7.

58. The nucleic acid molecule of any one of embodiments 51-57, wherein said RGN polypeptide has the amino acid sequence of SEQ ID NO: 7.

59. The nucleic acid molecule of any one of embodiments 45-58, wherein said guide RNA comprises a crRNA repeat having the nucleotide sequence of SEQ ID NO: 8 or 106 or a nucleotide sequence that differs from SEQ ID NO: 8 or 106 by 1 or 2 nucleotides and a tracrRNA having a nucleotide sequence having at least 90% sequence identity to SEQ ID NO: 9 or 107.

60. The nucleic acid molecule of any one of embodiments 45-58, wherein said guide RNA comprises a crRNA repeat having a nucleotide sequence that differs from SEQ ID NO: 8 or 106 by 1 nucleotide and a tracrRNA having a nucleotide sequence having at least 95% sequence identity to SEQ ID NO: 9 or 107.

61. The nucleic acid molecule of any one of embodiments 45-58, wherein said guide RNA comprises a crRNA repeat having the nucleotide sequence of SEQ ID NO: 8 or 106 and a tracrRNA having the nucleotide sequence of SEQ ID NO: 9 or 107.

62. The nucleic acid molecule of any one of embodiments 45-61, wherein said guide RNA is a single guide RNA.

63. The nucleic acid molecule of embodiment 62, wherein said single guide RNA has the nucleotide sequence of SEQ ID NO: 25 or 26.

64. The nucleic acid molecule of any one of embodiments 45-63, wherein said nucleic acid molecule encoding said guide RNA is operably linked to an RNA polymerase III promoter.

65. The nucleic acid molecule of embodiment 64, wherein said RNA polymerase III promoter is a U6 promoter.

66. The nucleic acid molecule of embodiment 65, wherein said U6 promoter is a truncated U6 promoter.

67. The nucleic acid molecule of embodiment 66, wherein said truncated U6 promoter has the nucleotide sequence set forth as SEQ ID NO: 89 or 128.

68. A vector comprising the nucleic acid molecule of any one of embodiments 45-50, wherein the nucleic acid molecule encodes the guide RNA.

69. A vector comprising the nucleic acid molecule of any one of embodiments 51-67, wherein the nucleic acid molecule encodes the guide RNA.

70. The vector of embodiment 68 or 69, wherein said vector is a viral vector.

71. The vector of embodiment 70, wherein said viral vector is a lentiviral vector, a baculoviral vector, or an adeno-associated viral (AAV) vector.

72. The vector of embodiment 71, wherein said viral vector is an AAV vector and comprises AAV inverted terminal repeats.

73. The vector of embodiment 72, wherein said AAV inverted terminal repeats are AAV2, AAV5 or AAV6 inverted terminal repeats.

74. The vector of any one of embodiments 69-73, wherein the vector further comprises a nucleic acid molecule encoding said RGN polypeptide.

75. The vector of embodiment 74, wherein the vector further comprises an RNA polymerase II promoter operably linked to the nucleic acid molecule encoding the RGN polypeptide.

76. The vector of embodiment 75, wherein said RNA polymerase II promoter is a constitutive promoter.

77. The vector of embodiment 76, wherein said constitutive promoter is selected from the group consisting of: a cytomegalovirus (CMV) promoter, a truncated CMV promoter, an elongation factor 1a short (EFS) promoter, and a JeT promoter.

78. The vector of embodiment 77, wherein said constitutive promoter is a JeT promoter.

79. The vector of embodiment 78, wherein said JeT promoter has the nucleotide sequence set forth as SEQ ID NO: 92.

80. The vector of embodiment 75, wherein said RNA polymerase II promoter is a tissue-specific promoter.

81. The vector of embodiment 780, wherein said tissue-specific promoter is a brain or neuron specific promoter.

82. The vector of embodiment 81, wherein said brain or neuron specific promoter is selected from the group consisting of: a human synapsin I (Syn) promoter, a 67 kDa glutamic acid decarboxylase (GAD67) promoter, a 65 kDa glutamic acid decarboxylase (GAD65) promoter, a homeobox Dlx5/6 promoter, a preprotachykinin 1 (Tac1) promoter, a neuron-specific enolase (NSE) promoter, a dopaminergic receptor 1 (Drd1a) promoter, a dopaminergic receptor 2 (DRD2) promoter, and a glial fibrillary acidic protein (GFAP) promoter.

83. The vector of embodiment 82, wherein said neuron specific promoter is a Syn promoter.

84. The vector of embodiment 83, wherein said Syn promoter has the nucleotide sequence set forth as SEQ ID NO: 93.

85. The vector of any one of embodiments 74-74, wherein said nucleic acid molecule encoding said RGN polypeptide comprises a polyadenylation (polyA) tail.

86. The vector of embodiment 85, wherein said polyA tail is a SV40 polyA tail or a bovine growth hormone (bGH) polyA tail.

87. The vector of embodiment 86, wherein said SV40 polyA tail has the nucleotide sequence set forth as SEQ ID NO: 94.

88. The vector of embodiment 86, wherein said bGH polyA tail has the sequence set forth as SEQ ID NO: 95.

89. The vector of embodiment 74 or 75, wherein said vector comprises: a truncated U6 promoter operably linked to said nucleic acid molecule encoding said guide RNA; a CMVeb promoter operably linked to said nucleic acid molecule encoding said RGN polypeptide; a c-Myc NLS at the N-terminus and C-terminus of said RGN polypeptide; an NLS linker protein connecting said c-Myc NLS and said RGN polypeptide; and an SV40 polyA tail.

90. The vector of embodiment 89, wherein said truncated U6 promoter has the sequence set forth as SEQ ID NO: 128, said CMVeb promoter has the sequence set forth as SEQ ID NO: 90, said c-Myc NLS has the sequence set forth as SEQ ID NO: 125, said NLS linker protein has the sequence set forth as SEQ ID NO: 127, and said SV40 polyA tail has the sequence set forth as SEQ ID NO: 94.

91. The vector of embodiment 89 or 90, wherein said sgRNA has the sequence set forth as SEQ ID NO: 26 and said nucleic acid molecule encoding said RGN polypeptide has the sequence set forth as SEQ ID NO: 88.

92. The vector of any one of embodiments 89-91, wherein said vector comprises the sequence set forth as SEQ ID NO: 123.

93. The vector of any one of embodiments 74-92, wherein said RGN polypeptide is operably linked to at least one nuclear localization signal.

94. The vector of embodiment 93, wherein said at least one nuclear localization signal comprises an SV40 nuclear localization signal.

95. The vector of embodiment 94, wherein said SV40 nuclear localization signal has the sequence set forth as SEQ ID NO: 86.

96. The vector of embodiment 93, wherein said at least one nuclear localization signal comprises a c-Myc nuclear localization signal.

97. The vector of embodiment 96, wherein said c-Myc nuclear localization signal has the sequence set forth as SEQ ID NO: 125.

98. The vector of any one of embodiments 93-97, wherein a NLS linker protein connects said RGN polypeptide and said at least one nuclear localization signal.

99. The vector of embodiment 98, wherein said NLS linker protein has the sequence set forth as SEQ ID NO: 127.

100. The vector of any one of embodiments 74-99, wherein said vector has the sequence set forth as any one of SEQ ID NOs: 32-39 or 121-123.

101. An RNA-guided nuclease (RGN) system comprising:
a) a guide RNA comprising having the nucleotide sequence of SEQ ID NO: 82 or 83 or a nucleotide sequence that differs from SEQ ID NO: 82 or 83 by 1 or 2 nucleotides, or a nucleic acid molecule encoding the guide RNA; and
b) an RGN polypeptide having an amino acid sequence having at least 90% sequence identity to SEQ ID NO: 3 or a nucleic acid molecule encoding the RGN polypeptide.

102. The RGN system of embodiment 101, wherein said guide RNA comprises a spacer having a nucleotide sequence that differs from SEQ ID NO: 82 or 83 by 2 nucleotides.

103. The RGN system of embodiment 101, wherein said guide RNA comprises a spacer having a nucleotide sequence that differs from SEQ ID NO: 82 or 83 by 1 nucleotide.

104. The RGN system of embodiment 101, wherein said guide RNA comprises a spacer having the nucleotide sequence of SEQ ID NO: 82 or 83.

105. The RGN system of any one of embodiments 101-104, wherein said guide RNA binds to a target sequence in a mutant huntingtin (mutHTT) allele.

106. The RGN system of embodiment 105, wherein said system is capable of binding and cleaving a target sequence in said mutHTT allele, and wherein the guide RNA is capable of forming a complex with the RGN polypeptide and directing the complex to the target sequence for binding and cleaving.

107. The RGN system of embodiment 105 or 106, wherein said target sequence has the nucleotide sequence of SEQ ID NO: 77 or 78.

108. The RGN system of any one of embodiments 101-107, wherein said RGN system is capable of recognizing a protospacer adjacent motif (PAM) having the sequence of NNNNCC created by a single nucleotide polymorphism (SNP) in exon 50 of the HTT gene.

109. The RGN system of any one of embodiments 101-108, wherein said RGN comprises a PAM-interacting domain that binds a protospacer adjacent motif (PAM) having the sequence of NNNNCC created by a single nucleotide polymorphism (SNP) in exon 50 of the HTT gene.

110. The RGN system of embodiment 109, wherein said PAM-interacting domain comprises an amino acid sequence having at least 95% sequence identity to SEQ ID NO: 133.

111. The RGN system of embodiment 109 or 110, wherein said PAM-interacting domain comprises the amino acid sequence set forth as SEQ ID NO: 133.

112. The RGN system of any one of embodiments 101-111, wherein said RGN polypeptide comprises at least one nuclease domain comprising an amino acid sequence having at least 95% sequence identity to any one of SEQ ID NOs: 143, 144, 145, and 146.

113. The RGN system of any one of embodiments 101-112, wherein said RGN system is not capable of cleaving a wild type HTT allele.

114. The RGN system of any one of embodiments 101-113, wherein said RGN polypeptide has an amino acid sequence having at least 95% sequence identity to SEQ ID NO: 3.

115. The RGN system of any one of embodiments 101-114, wherein said RGN polypeptide has the amino acid sequence of SEQ ID NO: 3.

116. The RGN system of any one of embodiments 101-115, wherein said RGN polypeptide comprises at least one nuclear localization signal.

117. The RGN system of embodiment 116, wherein said at least one nuclear localization signal comprises an SV40 nuclear localization signal.

118. The RGN system of embodiment 117, wherein said SV40 nuclear localization signal has the sequence set forth as SEQ ID NO: 86.

119. The RGN system of embodiment 116, wherein said at least one nuclear localization signal comprises a c-Myc nuclear localization signal.

120. The RGN system of embodiment 119, wherein said c-Myc nuclear localization signal has the sequence set forth as SEQ ID NO: 125.

121. The RGN system of any one of embodiments 116-120, wherein a NLS linker protein connects said RGN polypeptide and said at least one nuclear localization signal.

122. The RGN system of embodiment 121, wherein said NLS linker protein has the sequence set forth as SEQ ID NO: 127.

123. The RGN system of any one of embodiments 101-122, wherein the backbone of the guide RNA is 94 to 110 nucleotides in length.

124. The RGN system of any one of embodiments 101-123, wherein the backbone of the guide RNA comprises a nucleotide sequence having at least 80% sequence identity to SEQ ID NO: 142.

125. The RGN system of any one of embodiments 101-124, wherein said nucleic acid molecule encoding said RGN polypeptide is operably linked to an RNA polymerase II promoter.

126. The RGN system of embodiment 125, wherein said RNA polymerase II promoter is a constitutive promoter.

127. The RGN system of embodiment 126, wherein said constitutive promoter is selected from the group consisting of: a cytomegalovirus (CMV) promoter, a truncated CMV promoter, an elongation factor 1α short (EFS) promoter, and a JeT promoter.

128. The RGN system of embodiment 127, wherein said constitutive promoter is a JeT promoter.

129. The RGN system of embodiment 128, wherein said JeT promoter has the nucleotide sequence set forth as SEQ ID NO: 92.

130. The RGN system of embodiment 125, wherein said RNA polymerase II promoter is a tissue-specific promoter.

131. The RGN system of embodiment 130, wherein said tissue-specific promoter is a brain or neuron specific promoter.

132. The RGN system of embodiment 131, wherein said brain or neuron specific promoter is selected from the group consisting of: a human synapsin I (Syn) promoter, a 67 kDa glutamic acid decarboxylase (GAD67) promoter, a 65 kDa glutamic acid decarboxylase (GAD65) promoter, a homeobox Dlx5/6 promoter, a preprotachykinin 1 (Tac1) promoter, a neuron-specific enolase (NSE) promoter, a dopaminergic receptor 1 (Drd1a) promoter, a dopaminergic receptor 2 (DRD2) promoter, and glial fibrillary acidic protein (GFAP) promoter.

133. The RGN system of embodiment 132, wherein said neuron specific promoter is a Syn promoter.

134. The RGN system of embodiment 133, wherein said Syn promoter has the nucleotide sequence set forth as SEQ ID NO: 93.

135. The RGN system of any one of embodiments 101-134, wherein said nucleic acid molecule encoding said RGN polypeptide comprises a polyadenylation (polyA) tail.

136. The RGN system of embodiment 135, wherein said polyA tail is a SV40 polyA tail or a bovine growth hormone (bGH) polyA tail.

137. The RGN system of embodiment 136, wherein said SV40 polyA tail has the nucleotide sequence set forth as SEQ ID NO: 94.

138. The RGN system of embodiment 136, wherein said bGH polyA tail has the sequence set forth as SEQ ID NO: 95.

139. A ribonucleoprotein (RNP) complex comprising the RGN polypeptide and the guide RNA of said RGN system of any one of embodiments 101-138.

140. A nucleic acid molecule comprising or encoding a guide RNA that comprises a spacer having the nucleotide sequence of SEQ ID NO: 82 or 83 or a nucleotide sequence that differs from SEQ ID NO: 82 or 83 by 1 or 2 nucleotides.

141. The nucleic acid molecule of embodiment 140, wherein said guide RNA comprises a spacer having a nucleotide sequence that differs from SEQ ID NO: 82 or 83 by 2 nucleotides.

142. The nucleic acid molecule of embodiment 140, wherein said guide RNA comprises a spacer having a nucleotide sequence that differs from SEQ ID NO: 82 or 83 by 1 nucleotide.

143. The nucleic acid molecule of embodiment 140, wherein said guide RNA comprises a spacer having the nucleotide sequence of SEQ ID NO: 82 or 83.

144. The nucleic acid molecule of any one of embodiments 140-143, wherein said guide RNA binds to a target sequence in a mutant huntingtin (mutHTT) allele.

145. The nucleic acid molecule of embodiment 144, wherein said target sequence has the nucleotide sequence of SEQ ID NO: 77 or 78.

146. The nucleic acid molecule of any one of embodiments 140-145, wherein said guide RNA binds to an RNA-guided nuclease (RGN) polypeptide having an amino acid sequence having at least 90% sequence identity to SEQ ID NO: 3.

147. A nucleic acid molecule comprising or encoding a guide RNA that binds to a target sequence in a mutant huntingtin (mutHTT) allele, wherein said target sequence has the nucleotide sequence of SEQ ID NO: 77 or 78 and binds to an RNA-guided nuclease (RGN) polypeptide having an amino acid sequence having at least 90% sequence identity to SEQ ID NO: 3.

148. The nucleic acid molecule of embodiment 147, wherein said guide RNA comprises a spacer having the nucleotide sequence of SEQ ID NO: 82 or 83 or a nucleotide sequence that differs from SEQ ID NO: 82 or 83 by 1 or 2 nucleotides.

149. The nucleic acid molecule of embodiment 148, wherein said guide RNA comprises a spacer having a nucleotide sequence that differs from SEQ ID NO: 82 or 83 by 2 nucleotides.

150. The nucleic acid molecule of embodiment 148, wherein said guide RNA comprises a spacer having a nucleotide sequence that differs from SEQ ID NO: 82 or 83 by 1 nucleotide.

151. The nucleic acid molecule of embodiment 148, wherein said guide RNA comprises a spacer having the nucleotide sequence of SEQ ID NO: 82 or 83.

152. The nucleic acid molecule of any one of embodiments 146-151, wherein said RGN polypeptide has an amino acid sequence having at least 95% sequence identity to SEQ ID NO: 3.

153. The nucleic acid molecule of any one of embodiments 146-152, wherein said RGN polypeptide has the amino acid sequence of SEQ ID NO: 3.

154. The nucleic acid molecule of any one of embodiments 140-153, wherein said guide RNA comprises a crRNA repeat having the nucleotide sequence of SEQ ID NO: 4 or a nucleotide sequence that differs from SEQ ID NO: 4 by 1 or 2 nucleotides and a tracrRNA having a nucleotide sequence having at least 90% sequence identity to SEQ ID NO: 5.

155. The nucleic acid molecule of any one of embodiments 140-153, wherein said guide RNA comprises a crRNA repeat having a nucleotide sequence that differs from SEQ ID NO: 4 by 1 nucleotide and a tracrRNA having a nucleotide sequence having at least 95% sequence identity to SEQ ID NO: 5.

156. The nucleic acid molecule of any one of embodiments 140-153, wherein said guide RNA comprises a crRNA repeat having the nucleotide sequence of SEQ ID NO: 4 and a tracrRNA having the nucleotide sequence of SEQ ID NO: 5.

157. The nucleic acid molecule of any one of embodiments 140-156, wherein said guide RNA is a single guide RNA.

158. The nucleic acid molecule of embodiment 157, wherein said single guide RNA has the nucleotide sequence of SEQ ID NO: 27 or 28.

159. The nucleic acid molecule of any one of embodiments 140-158, wherein said nucleic acid molecule encoding said guide RNA is operably linked to an RNA polymerase III promoter.

160. The nucleic acid molecule of embodiment 159, wherein said RNA polymerase III promoter is a U6 promoter.

161. The nucleic acid molecule of embodiment 160, wherein said U6 promoter is a truncated U6 promoter.

162. The nucleic acid molecule of embodiment 161, wherein said truncated U6 promoter has the nucleotide sequence set forth as SEQ ID NO: 89 or 128.

163. A vector comprising the nucleic acid molecule of any one of embodiments 140-145, wherein the nucleic acid molecule encodes the guide RNA.

164. A vector comprising the nucleic acid molecule of any one of embodiments 146-162, wherein the nucleic acid molecule encodes the guide RNA.

165. The vector of embodiment 163 or 164, wherein said vector is a viral vector.

166. The vector of embodiment 165, wherein said viral vector is a lentiviral vector, a baculoviral vector, or an adeno-associated viral (AAV) vector.

167. The vector of embodiment 166, wherein said viral vector is an AAV vector and comprises AAV inverted terminal repeats.

168. The vector of embodiment 167, wherein said AAV inverted terminal repeats are AAV2, AAV5 or AAV6 inverted terminal repeats.

169. The vector of any one of embodiments 164-168, wherein the vector further comprises a nucleic acid molecule encoding said RGN polypeptide.

170. The vector of embodiment 169, wherein the vector further comprises an RNA polymerase II promoter operably linked to the nucleic acid molecule encoding the RGN polypeptide.

171. The vector of embodiment 170, wherein said RNA polymerase II promoter is a constitutive promoter.

172. The vector of embodiment 171, wherein said constitutive promoter is selected from the group consisting of: a cytomegalovirus (CMV) promoter, a truncated CMV promoter, an elongation factor 1α short (EFS) promoter, and a JeT promoter.

173. The vector of embodiment 172, wherein said constitutive promoter is a JeT promoter.

174. The vector of embodiment 173, wherein said JeT promoter has the nucleotide sequence set forth as SEQ ID NO: 92.

175. The vector of embodiment 170, wherein said RNA polymerase II promoter is a tissue-specific promoter.

176. The vector of embodiment 175, wherein said tissue-specific promoter is a brain or neuron specific promoter.

177. The vector of embodiment 176, wherein said brain or neuron specific promoter is selected from the group consisting of: a human synapsin I (Syn) promoter, a 67 kDa glutamic acid decarboxylase (GAD67) promoter, a 65 kDa glutamic acid decarboxylase (GAD65) promoter, a homeobox Dlx5/6 promoter, a preprotachykinin 1 (Tac1) promoter, a neuron-specific enolase (NSE) promoter, a dopaminergic receptor 1 (Drd1a) promoter, a dopaminergic receptor 2 (DRD2) promoter, and a glial fibrillary acidic protein (GFAP) promoter.

178. The vector of embodiment 177, wherein said neuron specific promoter is a Syn promoter.

179. The vector of embodiment 178, wherein said Syn promoter has the nucleotide sequence set forth as SEQ ID NO: 93.

180. The vector of any one of embodiments 169-179, wherein said nucleic acid molecule encoding said RGN polypeptide comprises a polyadenylation (polyA) tail.

181. The vector of embodiment 180, wherein said polyA tail is a SV40 polyA tail or a bovine growth hormone (bGH) polyA tail.

182. The vector of embodiment 181, wherein said SV40 polyA tail has the nucleotide sequence set forth as SEQ ID NO: 94.

183. The vector of embodiment 181, wherein said bGH polyA tail has the sequence set forth as SEQ ID NO: 95.

184. The vector of any one of embodiments 169-183, wherein said RGN polypeptide is operably linked to at least one nuclear localization signal.

185. The vector of embodiment 184, wherein said at least one nuclear localization signal comprises an SV40 nuclear localization signal.

186. The vector of embodiment 185, wherein said SV40 nuclear localization signal has the sequence set forth as SEQ ID NO: 86.

187. The vector of embodiment 184, wherein said at least one nuclear localization signal comprises a c-Myc nuclear localization signal.

188. The vector of embodiment 187, wherein said c-Myc nuclear localization signal has the sequence set forth as SEQ ID NO: 125.

189. The vector of any one of embodiments 184-188, wherein a NLS linker protein connects said RGN polypeptide and said at least one nuclear localization signal.

190. The vector of embodiment 189, wherein said NLS linker protein has the sequence set forth as SEQ ID NO: 127.

191. A cell comprising the nucleic acid molecule of any one of embodiments 45-67 and 140-162 or the vector of any one of embodiments 68-100 and 163-190.

192. A pharmaceutical composition comprising the nucleic acid molecule of any one of embodiments 45-67 and 140-162, the vector of any one of embodiments 68-100 and 163-190, the RGN system of any one of embodiments 1-43 and 101-138, or the RNP complex of embodiment 44 or 139.

193. The pharmaceutical composition of embodiment 192 having a purity of at least 95%.

194. The pharmaceutical composition of embodiment 192 or 193 having undetectable levels of endotoxin or other impurities.

195. The pharmaceutical composition of any one of embodiments 192-194, further comprising poloxamer 188.

196. The pharmaceutical composition of any of embodiments 192-195 that is in solution.

197. The pharmaceutical composition of any of embodiments 192-195 that is lyophilized or freeze-dried.

198. A vector comprising the RGN system of any one of embodiments 1-43.

199. A vector comprising the RGN system of any one of embodiments 101-138.

200. The vector of embodiment 198 or 199, wherein said vector comprises adeno-associated vector (AAV) inverted terminal repeats.

201. The vector of embodiment 200, wherein said AAV inverted terminal repeats are AAV2, AAV5, or AAV6 inverted terminal repeats.

202. The vector of embodiment 201, wherein said AAV inverted terminal repeats are AAV5 inverted terminal repeats.

203. Use of the nucleic acid molecule of any one of embodiments 45-67 and 140-162, the vector of any one of embodiments 68-100, 163-190, and 198-202, the RGN system of any one of embodiments 1-43 and 101-138, or the RNP complex of embodiment 44 or 139 for reducing the level of mutHTT mRNA and/or mutHTT protein in a cell.

204. Use of the nucleic acid molecule of any one of embodiments 45-67 and 140-162, the vector of any one of embodiments 68-100, 163-190, and 198-202, the RGN system of any one of embodiments 1-43 and 101-138, or the RNP complex of embodiment 44 or 139 for treating Huntington's disease.

205. A method for cleaving a mutant huntingtin (mutHTT) allele in a cell, wherein said mutHTT allele comprises a first single nucleotide polymorphism (SNP) allele in exon 50, wherein a protospacer adjacent motif (PAM) having the nucleotide sequence of NNRYA comprises said first SNP allele, wherein said method comprises introducing into the cell:
a) an RNA-guided nuclease (RGN) polypeptide having at least 90% sequence identity to SEQ ID NO: 7 or a nucleic acid molecule encoding said RGN polypeptide, and
   i) the nucleic acid molecule comprising or encoding a guide RNA of any one of embodiments 45-67; or
   ii) the vector of any one of embodiments 68-73;
b) the vector of any one of embodiments 74-100, 198, and 200-202;
c) the RGN system of any one of embodiments 1-43; or
d) the RNP complex of embodiment 44.

206. A method for cleaving a mutant huntingtin (mutHTT) allele in a cell, wherein said mutHTT allele comprises a first single nucleotide polymorphism (SNP) allele in exon 50, wherein a protospacer adjacent motif (PAM) having the nucleotide sequence of NNNNCC comprises said first SNP allele, wherein said method comprises introducing into the cell:
a) an RNA-guided nuclease (RGN) polypeptide having at least 90% sequence identity to SEQ ID NO: 3 or a nucleic acid molecule encoding said RGN polypeptide, and
   i) the nucleic acid molecule comprising or encoding a guide RNA of any one of embodiments 140-162; or
   ii) the vector of any one of embodiments 163-168;
b) the vector of any one of embodiments 169-190, and 199-202;
c) the RGN system of any one of embodiments 101-138; or
d) the RNP complex of embodiment 139.

207. The method of embodiment 205 or 206, wherein said RGN polypeptide is capable of recognizing said PAM and cleaving said mutHTT allele

208. The method of any one of embodiments 205-207, wherein said mutHTT allele has at least 36 CAG repeats in exon 1.

209. The method of any one of embodiments 205-207, wherein said mutHTT allele has at least 40 CAG repeats in exon 1.

210. The method of any one of embodiments 205-209, wherein said cell has been assayed to determine whether said mutHTT allele comprises said first SNP allele prior to the introduction of said RGN polypeptide or said nucleic acid molecule encoding said RGN polypeptide and said guide RNA or said nucleic acid molecule encoding said guide RNA, said vector, said RGN system, or said RNP complex.

211. The method of any one of embodiments 205-210, wherein said cell comprises a wild-type HTT (wtHTT) allele comprising a second SNP allele where said PAM is not present, and said cell is thereby heterozygous for the SNP.

212. The method of embodiment 211, wherein said cell has been assayed to determine whether said cell is heterozygous for the SNP prior to the introduction of said RGN polypeptide or said nucleic acid molecule encoding said RGN polypeptide and said guide RNA or said nucleic acid molecule encoding said guide RNA, said vector, said RGN system, or said RNP complex.

213. The method of any one of embodiments 205-212, wherein said mutHTT allele is edited, thereby creating a genetically modified cell comprising said edited mutHTT allele.

214. The method of embodiment 213, wherein said editing comprises introducing an insertion and/or deletion (INDEL) at or near said SNP.

215. The method of embodiment 213, wherein said editing comprises introducing a premature stop codon at or near said SNP.

216. The method of any one of embodiments 213-215, wherein said genetically modified cell is a genetically modified stem cell.

217. The method of embodiment 216, wherein said genetically modified stem cell is a genetically modified induced pluripotent stem cell (iPSC) or a genetically modified mesenchymal stem cell (MSC).

218. The method of embodiment 217, wherein said method further comprises differentiating the genetically modified iPSC or MSC into a neuronal cell.

219. The method of any one of embodiments 213-218, wherein a level of mutHTT mRNA is reduced in said genetically modified cell as compared to a level of HTT mRNA in a non-genetically modified cell or to a level of wild type HTT mRNA.

220. The method of embodiment 219, wherein the level of mutHTT mRNA is reduced by at least 40% as compared to the level of HTT mRNA in a non-genetically modified cell or to the level of wild type HTT mRNA.

221. The method of any one of embodiments 213-220, wherein a level of mutHTT protein encoded by said mutHTT allele is reduced in said genetically modified cell as compared to a level of HTT protein in a non-genetically modified cell or to a level of wild type HTT protein.

222. The method of embodiment 221, wherein the level of mutHTT protein is reduced by at least 40% as compared to the level of HTT protein in a non-genetically modified cell or to the level of wild type HTT protein.

223. The method of any one of embodiments 213-222, further comprising selecting said genetically modified cell.

224. A genetically modified cell produced by the method of embodiment 223.

225. The method of any one of embodiments 205-223, wherein said introducing comprises administering a composition comprising said RGN polypeptide or said nucleic acid molecule encoding said RGN polypeptide and said guide RNA or said nucleic acid molecule encoding said guide RNA, said vector, said RGN system, or said RNP complex, to a subject comprising said cell; or administering said genetically modified cell of any one of embodiments 213-223.

226. The method of embodiment 225, wherein the cell is a eukaryotic cell.

227. The method of embodiment 226, wherein the eukaryotic cell is a mammalian cell.

228. The method of embodiment 227, wherein the mammalian cell is a human cell.

229. The method of embodiment 227 or 228, wherein the mammalian cell or human cell is a stem cell.

230. The method of embodiment 227 or 228, wherein the mammalian cell or human cell is a forebrain neuron, a striatal neuron, a medium spiny neuron, a cortical neuron, or a glial cell.

231. The method of embodiment 227 or 228, wherein the mammalian cell or human cell is present in putamen, caudate, striatum, cerebral cortex, globus pallidus, hippocampus, amygdala, thalamus, hypothalamus, subthalamic nucleus, substantia nigra, cerebellum, brainstem, or a combination thereof.

232. A method for ameliorating or delaying the onset of one or more symptoms of Huntington's disease (HD) in a subject in need thereof, wherein said subject comprises a mutant huntingtin (mutHTT) allele comprising:
a) at least 36 CAG repeats in exon 1; and
b) a first single nucleotide polymorphism (SNP) allele in exon 50, wherein a protospacer adjacent
motif (PAM) having the nucleotide sequence of NNRYA comprises said first SNP allele;
   wherein said method comprises administering to said subject:
a) an RNA-guided nuclease (RGN) polypeptide having at least 90% sequence identity to SEQ ID NO: 7 or a nucleic acid molecule encoding said RGN polypeptide, and
   i) the nucleic acid molecule comprising or encoding a guide RNA of any one of embodiments 45-67; or
   ii) the vector of any one of embodiments 68-73;
b) the vector of any one of embodiments 74-100, 198, and 200-202;
c) the RGN system of any one of embodiments 1-43; or
d) the RNP complex of embodiment 44;
   and wherein the level of a mutHTT protein encoded by said mutHTT allele is reduced as compared to a level of HTT protein of a control subject or a level of wild type HTT protein.

233. A method for ameliorating or delaying the onset of one or more symptoms of Huntington's disease (HD) in a subject in need thereof, wherein said subject comprises a mutant huntingtin (mutHTT) allele comprising:
a) at least 36 CAG repeats in exon 1; and
b) a first single nucleotide polymorphism (SNP) allele in exon 50, wherein a protospacer adjacent motif (PAM) having the nucleotide sequence of NNNNCC comprises said first SNP allele;
   wherein said method comprises administering to said subject:
a) an RNA-guided nuclease (RGN) polypeptide having at least 90% sequence identity to SEQ ID NO: 3 or a nucleic acid molecule encoding said RGN polypeptide, and
   i) the nucleic acid molecule comprising or encoding a guide RNA of any one of embodiments 140-162; or
   ii) the vector of any one of embodiments 163-168;
b) the vector of any one of embodiments 169-190, and 199-202;
c) the RGN system of any one of embodiments 101-138; or
d) the RNP complex of embodiment 139;
   and wherein the level of a mutHTT protein encoded by said mutHTT allele is reduced as compared to a level of HTT protein of a control subject or a level of wild type HTT protein.

234. The method of embodiment 232 or 233, wherein said RGN recognizes said PAM and cleaves and edits said mutHTT allele

235. The method of any one of embodiments 232-234, wherein said administering comprises intrastriatal, intraparenchymal, intrathecal, intracerebral, intracerebroventricular, intrathalamic, or intra-cisterna magna injection.

236. The method of any one of embodiments 232-235, wherein said subject has been assayed to determine whether said mutHTT allele comprises said first SNP allele comprising said PAM prior to the administration of said RGN polypeptide or said nucleic acid molecule encoding said RGN polypeptide and said guide RNA or said nucleic acid molecule encoding said guide RNA, said vector, said RGN system, or said RNP complex.

237. The method of any one of embodiments 232-236, wherein the subject comprises a wild-type HTT (wtHTT) allele comprising a second SNP allele where said PAM is not present, and said subject is thereby heterozygous for the SNP.

238. The method of embodiment 237, wherein said subject has been assayed to determine whether said subject is heterozygous for the SNP prior to the administration of said RGN polypeptide or said nucleic acid molecule encoding said RGN polypeptide and said guide RNA or said nucleic acid molecule encoding said guide RNA, said vector, said RGN system, or said RNP complex.

239. The method of any one of embodiments 232-238, wherein said PAM is present only on the mutHTT allele and not the wild-type HTT allele.

240. A method of ameliorating or delaying the onset of one or more symptoms of Huntington's disease (HD) in a subject in need thereof, wherein said subject comprises a mutant huntingtin (mutHTT) allele comprising:
a) at least 36 CAG repeats in exon 1; and
b) a first single nucleotide polymorphism (SNP) allele in exon 50, wherein said SNP allele comprises a thymine at a position corresponding to position 151 of SEQ ID NO: 1;
   wherein said method comprises administering by intrastriatal injection into said subject an AAV5 vector comprising:
a) a first nucleic acid molecule encoding an RNA-guided nuclease (RGN) polypeptide having the amino acid sequence of SEQ ID NO: 7; and
b) a second nucleic acid molecule encoding a guide RNA having the nucleotide sequence of SEQ ID NO: 25 or 26;
   and wherein 4-weeks post-administration of said AAV5 vector, said subject has a decrease in a level of mutHTT protein encoded by said mutHTT allele as compared to a level of HTT protein of a control subject or a level of wild type HTT protein.

241. A method of ameliorating or delaying the onset of one or more symptoms of Huntington's disease (HD) in a subject in need thereof, wherein said subject comprises a mutant huntingtin (mutHTT) allele comprising:
a) at least 36 CAG repeats in exon 1; and
b) a first single nucleotide polymorphism (SNP) allele in exon 50, wherein said first SNP allele comprises a cytosine at a position corresponding to position 151 of SEQ ID NO: 2;
   wherein said method comprises administering by intrastriatal injection into said subject an AAV5 vector comprising:
a) a first nucleic acid molecule encoding an RNA-guided nuclease (RGN) polypeptide having the amino acid sequence of SEQ ID NO: 3; and
b) a second nucleic acid molecule encoding a guide RNA having the nucleotide sequence of SEQ ID NO: 27 or 28;
   and wherein 4 weeks post-administration of said AAV5 vector, said subject has a decrease in a level of mutHTT protein encoded by said mutHTT allele as compared to a level of HTT protein of a control subject or a level of wild type HTT protein.

242. A method of ameliorating or delaying the onset of one or more symptoms of Huntington's disease (HD) in a subject in need thereof, said method comprises:
a) selecting a subject comprising a mutant huntingtin (mutHTT) allele comprising:
   i) at least 36 CAG repeats in exon 1;
   ii) a first single nucleotide polymorphism (SNP) allele in exon 50, wherein said SNP allele comprises a thymine at a position corresponding to position 151 of SEQ ID NO: 1; and
b) administering by intrastriatal injection into said subject an AAV5 vector comprising:
   i) a first nucleic acid molecule encoding an RNA-guided nuclease (RGN) polypeptide having the amino acid sequence of SEQ ID NO: 7; and
   ii) a second nucleic acid molecule encoding a guide RNA having the nucleotide sequence of SEQ ID NO: 25 or 26;
      and wherein 4 weeks post-administration of said AAV5 vector, said subject has a decrease in a level of mutHTT protein encoded by said mutHTT allele as compared to a level of HTT protein of a control subject or a level of wild type HTT protein.

243. A method of ameliorating or delaying the onset of one or more symptoms of Huntington's disease (HD) in a subject in need thereof, wherein said method comprises:
a) selecting a subject comprising a mutant huntingtin (mutHTT) allele comprising:
   i) at least 36 CAG repeats in exon 1; and
   ii) a first single nucleotide polymorphism (SNP) allele in exon 50, wherein said SNP allele comprises a cytosine at a position corresponding to position 151 of SEQ ID NO: 2; and
b) administering by intrastriatal injection into said subject an AAV5 vector comprising:
   i) a first nucleic acid molecule encoding an RNA-guided nuclease (RGN) polypeptide having the amino acid sequence of SEQ ID NO: 3; and
   ii) a second nucleic acid molecule encoding a guide RNA having the nucleotide sequence of SEQ ID NO: 27 or 28;
      and wherein 4 weeks post-administration of said AAV5 vector, said subject has a decrease in a level of mutHTT protein encoded by said mutHTT allele as compared to a level of HTT protein of a control subject or a level of wild type HTT protein.

244. The method of any one of embodiments 240-243, wherein said RGN polypeptide recognizes and cleaves and edits said mutHTT allele

245. The method of any one of embodiments 240-244, wherein said editing comprises introducing an INDEL at or near said SNP.

246. The method of any one of embodiments 240-244, wherein said editing comprises introducing a premature stop codon at or near said SNP.

247. The method of any one of embodiments 240-246, wherein said mutHTT allele has at least 40 CAG repeats in exon 1.

248. The method of any one of embodiments 240-246, wherein said mutHTT allele has at least 56 CAG repeats in exon 1 and wherein said subject is younger than 18 years of age.

249. The method of any one of embodiments 240-248, wherein said administering occurs prior to onset of symptoms of Huntington's disease.

250. The method of any one of embodiments 240-249, wherein said method comprises preventing the onset of one or more symptoms of Huntington's disease.

251. The method of any one of embodiments 240-250, wherein said subject has at least one symptom of Huntington's disease.

252. The method of any of embodiments 240-251, wherein a decrease in a level of mutant HTT mRNA of at least 40% is observed as compared to a level of HTT mRNA of a control subject or a level of wild type HTT mRNA.

253. The method of any one of embodiments 240-252, wherein a decrease in the level of mutHTT protein is observed by 12 weeks after administration of said vector.

254. The method of embodiment 253, wherein at least a 40% decrease in the level of mutHTT protein is observed as compared to a level of HTT protein of a control subject or a level of wild type HTT protein.

255. The method of any of embodiments 240-254, wherein a decrease in a level of mutHTT mRNA is observed in at least 50% of the striatal cells in said subject.

256. The method of any of embodiments 240-255, wherein a decrease in the level of mutHTT protein is observed in at least 50% of the striatal cells in said subject.

257. The method of any of embodiments 240-256, wherein only the mutant HTT allele is edited.

258. A method for cleaving a mutant huntingtin (mutHTT) allele in a cell, wherein said mutHTT allele comprises a first single nucleotide polymorphism (SNP) allele in exon 50, wherein a protospacer adjacent motif (PAM) comprises said first SNP allele, wherein said method comprises introducing into the cell: (i) an RNA-guided nuclease (RGN) polypeptide or a nucleic acid molecule encoding said RGN polypeptide, and (ii) a guide RNA or a nucleic acid molecule encoding said guide RNA.

259. The method of embodiment 258, wherein said RGN polypeptide is capable of recognizing said PAM and cleaving said mutHTT allele.

260. The method of embodiment 258 or 259, wherein said mutHTT allele has at least 36 CAG repeats in exon 1.

261. The method of embodiment 258 or 259, wherein said mutHTT allele has at least 40 CAG repeats in exon 1.

262. The method of any one of embodiments 258-261, wherein said cell has been assayed to determine whether said mutHTT allele comprises said first SNP allele prior to the introduction of (i) said RGN polypeptide or said nucleic acid molecule encoding said RGN polypeptide, and (ii) said guide RNA or said nucleic acid molecule encoding said guide RNA.

263. The method of any one of embodiments 258-262, wherein said cell comprises a wild-type HTT (wtHTT) allele comprising a second SNP allele where said PAM is not present, and said cell is thereby heterozygous for the SNP.

264. The method of embodiment 263, wherein said cell has been assayed to determine whether said cell is heterozygous for the SNP prior to the introduction of (i) said RGN polypeptide or said nucleic acid molecule encoding said RGN polypeptide, and (ii) said guide RNA or said nucleic acid molecule encoding said guide RNA.

265. The method of any one of embodiments 258-264, wherein said mutHTT allele is edited, thereby creating a genetically modified cell comprising said edited mutHTT allele.

266. The method of embodiment 265, wherein said editing comprises introducing an insertion and/or deletion (INDEL) at or near said SNP.

267. The method of embodiment 265, wherein said editing comprises introducing a premature stop codon at or near said SNP.

268. The method of any one of embodiments 265-267, wherein said genetically modified cell is a genetically modified stem cell.

269. The method of embodiment 268, wherein said genetically modified stem cell is a genetically modified induced pluripotent stem cell (iPSC) or a genetically modified mesenchymal stem cell (MSC).

270. The method of embodiment 269, wherein said method further comprises differentiating the genetically modified iPSC or MSC into a neuronal cell.

271. The method of any one of embodiments 265-270, wherein a level of mutHTT mRNA is reduced in said genetically modified cell as compared to a level of HTT mRNA in a non-genetically modified cell or to a level of wild type HTT mRNA.

272. The method of embodiment 271, wherein the level of mutHTT mRNA is reduced by at least 40% compared to the level of HTT mRNA in a non-genetically modified cell or to the level of wild type HTT mRNA.

273. The method of any one of embodiments 265-272, wherein a level of mutHTT protein encoded by said mutHTT allele is reduced in said genetically modified cell as compared to a level of HTT protein in a non-genetically modified cell or to a level of wild type HTT protein.

274. The method of embodiment 273, wherein the level of mutHTT protein encoded by said mutHTT allele is reduced by at least 40% as compared to the level of HTT protein in a non-genetically modified cell or to the level of wild type HTT protein.

275. The method of any one of embodiments 265-274, further comprising selecting said genetically modified cell.

276. A genetically modified cell produced by the method of embodiment 275.

277. The method of any one of embodiments 258-274, wherein said introducing comprises administering a composition comprising (i) said RGN polypeptide or said nucleic acid molecule encoding said RGN polypeptide, and (ii) said guide RNA or said nucleic acid molecule encoding said guide RNA to a subject comprising said cell; or administering said genetically modified cell of any one of embodiments 265-276.

278. The method of embodiment 277, wherein the cell is a eukaryotic cell.

279. The method of embodiment 278, wherein the eukaryotic cell is a mammalian cell.

280. The method of embodiment 279, wherein the mammalian cell is a human cell.

281. The method of embodiment 279 or 280, wherein the mammalian cell or human cell is a stem cell.

282. The method of embodiment 279 or 280, wherein the mammalian cell or human cell is a forebrain neuron, a striatal neuron, a medium spiny neuron, a cortical neuron, or a glial cell.

283. The method of embodiment 279 or 280, wherein the mammalian cell or human cell is present in putamen, caudate, striatum, cerebral cortex, globus pallidus, hippocampus, amygdala, thalamus, hypothalamus, subthalamic nucleus, substantia nigra, cerebellum, brainstem, or a combination thereof.

284. A method for ameliorating or delaying the onset of one or more symptoms of Huntington's disease (HD) in a subject in need thereof, wherein said subject comprises a mutant huntingtin (mutHTT) allele comprising:
a) at least 36 CAG repeats in exon 1; and
b) a first single nucleotide polymorphism (SNP) allele in exon 50, wherein a protospacer adjacent motif (PAM) comprises said first SNP allele;
   wherein said method comprises administering to said subject (i) an RNA-guided nuclease (RGN) polypeptide or a nucleic acid molecule encoding said RGN polypeptide, and (ii) a guide RNA or a nucleic acid molecule encoding said guide RNA, and a level of a mutHTT protein encoded by said mutHTT allele is reduced as compared to a level of HTT protein in a control subject or a level of wild type HTT protein.

285. The method of embodiment 284, wherein said RGN polypeptide recognizes said PAM and cleaves and edits said mutHTT allele

286. The method of embodiment 284 or 285, wherein said mutHTT allele has at least 40 CAG repeats in exon 1.

287. The method of any one of embodiments 284-286, wherein said mutHTT allele has at least 56 CAG repeats in exon 1 and wherein said subject is younger than 18 years of age.

288. The method of any one of embodiments 284-287, wherein said administering occurs prior to onset of symptoms of Huntington's disease.

289. The method of any one of embodiments 284-288, wherein said method comprises preventing the onset of one or more symptoms of Huntington's disease.

290. The method of any one of embodiments 284-289, wherein said subject has at least one symptom of Huntington's disease.

291. The method of any one of embodiment 284-290, wherein said administering comprises intrastriatal, intraparenchymal, intrathecal, intracerebral, intracerebroventricular, intrathalamic, or intra-cisterna magna injection.

292. The method of any one of embodiments 284-291, wherein said subject has been assayed to determine whether said mutHTT allele comprising said PAM comprises said first SNP allele prior to the administration of (i) said RGN polypeptide or said nucleic acid molecule encoding said RGN polypeptide, and (ii) said guide RNA or said nucleic acid molecule encoding said guide RNA.

293. The method of any one of embodiments 284-292, wherein the subject comprises a wild-type HTT (wtHTT) allele comprising a second SNP allele where said PAM is not present, and said subject is thereby heterozygous for the SNP.

294. The method of embodiment 293, wherein said subject has been assayed to determine whether said subject is heterozygous for the SNP prior to the administration of (i) said RGN polypeptide or said nucleic acid molecule encoding said RGN polypeptide, and (ii) said guide RNA or said nucleic acid molecule encoding said guide RNA.

295. The method of any one of embodiments 284-293, wherein a level of mutHTT mRNA is reduced by at least 40% as compared to a level of HTT mRNA in a control subject or a level of wild type HTT mRNA.

296. The method of any one of embodiments 284-295, wherein a level of mutHTT protein is reduced by at least 40% as compared to a level of HTT protein in a control subject or a level of wild type HTT protein.

297. The method of any one of embodiments 284-296, wherein a decrease in a level of mutHTT protein is observed by 12 weeks after administration.

298. The method of any of embodiments 284-297, wherein a decrease in a level of mutHTT protein is observed in at least 50% of the striatal cells in said subject.

299. The method of any one of embodiments 258-298, wherein said PAM has a nucleotide sequence selected from the group consisting of: NNNNCC, NNRYA, NNGRR, and NNGG.

300. The method of embodiment 299, wherein the PAM sequence NNRYA comprises said first SNP allele, and wherein said first SNP allele is a thymine at a position corresponding to position 151 of SEQ ID NO: 1.

301. The method of embodiment 300, wherein said RGN polypeptide comprises an amino acid sequence having at least 90% sequence identity to SEQ ID NO: 7.

302. The method of embodiment 300 or 301, wherein said RGN polypeptide comprises an amino acid sequence having at least 95% sequence identity to SEQ ID NO: 7.

303. The method of any one of embodiments 300-302, wherein said RGN polypeptide comprises the amino acid sequence of SEQ ID NO: 7.

304. The method of any one of embodiments 300-303, wherein said guide RNA comprises a crRNA repeat having the nucleotide sequence of SEQ ID NO: 8 or 106 or a nucleotide sequence that differs from SEQ ID NO: 8 or 106 by 1 or 2 nucleotides and a tracrRNA having a nucleotide sequence having at least 90% sequence identity to SEQ ID NO: 9 or 107.

305. The method of any one of embodiments 300-303, wherein said guide RNA comprises a crRNA repeat having a nucleotide sequence that differs from SEQ ID NO: 8 or 106 by 1 nucleotide and a tracrRNA having a nucleotide sequence having at least 95% sequence identity to SEQ ID NO: 9 or 107.

306. The method of any one of embodiments 300-303, wherein said guide RNA comprises a crRNA repeat having the nucleotide sequence of SEQ ID NO: 8 or 106 and a tracrRNA having the nucleotide sequence of SEQ ID NO: 9 or 107.

307. The method of any one of embodiments 300-306, wherein said guide RNA comprises a spacer comprising a nucleotide sequence having complementarity with a target sequence having the nucleotide sequence of SEQ ID NO: 75 or 76.

308. The method of embodiment 307, wherein said spacer has the nucleotide sequence of SEQ ID NO: 80 or 81 or a nucleotide sequence that differs from SEQ ID NO: 80 or 81 by 1 or 2 nucleotides.

309. The method of embodiment 308, wherein said spacer has a nucleotide sequence that differs from SEQ ID NO: 80 or 81 by 2 nucleotides.

310. The method of embodiment 308, wherein said spacer has a nucleotide sequence that differs from SEQ ID NO: 80 or 81 by 1 nucleotide.

311. The method of embodiment 308, wherein said spacer has the nucleotide sequence of SEQ ID NO: 80 or 81.

312. The method of any one of embodiments 300-311, wherein said guide RNA is a single guide RNA.

313. The method of embodiment 312, wherein said single guide RNA has the nucleotide sequence of SEQ ID NO: 25 or 26.

314. The method of any one of embodiments 258-275 and 284-313, wherein said method comprises introducing a vector comprising said nucleic acid molecule encoding said RGN polypeptide, and said nucleic acid molecule encoding said guide RNA, and wherein said vector comprises: a truncated U6 promoter regulating the expression of a sgRNA; a CMVeb promoter regulating the expression of an RGN polypeptide; a c-Myc NLS at the N-terminus and C-terminus of said RGN polypeptide; an NLS linker protein connecting said c-Myc NLS to said RGN polypeptide; and an SV40 polyA tail.

315. The method of embodiment 314, wherein said truncated U6 promoter has the sequence set forth as SEQ ID NO: 128, said CMVeb promoter has the sequence set forth as SEQ ID NO: 90, said c-Myc NLS has the sequence set forth as SEQ ID NO: 125, said NLS linker protein has the sequence set forth as SEQ ID NO: 127, and said SV40 polyA tail has the sequence set forth as SEQ ID NO: 94.

316. The method of embodiment 314 or 315, wherein said sgRNA has the sequence set forth as SEQ ID NO: 26 and said nucleic acid molecule encoding said RGN polypeptide has the sequence set forth as SEQ ID NO: 88.

317. The method of any one of embodiments 314-316, wherein said vector comprises the sequence set forth as SEQ ID NO: 123.

318. The method of any one of embodiments 284-313, wherein said method comprises administering to said subject a vector comprising said nucleic acid molecule encoding said RGN polypeptide, and said nucleic acid molecule encoding said guide RNA, and wherein said vector comprises: a truncated U6 promoter regulating the expression of a sgRNA; a CMVeb promoter regulating the expression of an RGN polypeptide; a c-Myc NLS at the N-terminus and C-terminus of said RGN polypeptide; an NLS linker protein connecting said c-Myc NLS to said RGN polypeptide; and an SV40 polyA tail.

319. The method of embodiment 318, wherein said truncated U6 promoter has the sequence set forth as SEQ ID NO: 128, said CMVeb promoter has the sequence set forth as SEQ ID NO: 90, said c-Myc NLS has the sequence set forth as SEQ ID NO: 125, said NLS linker protein has the sequence set forth as SEQ ID NO: 127, and said SV40 polyA tail has the sequence set forth as SEQ ID NO: 94.

320. The method of embodiment 318 or 319, wherein said sgRNA has the sequence set forth as SEQ ID NO: 26 and said nucleic acid molecule encoding said RGN has the sequence set forth as SEQ ID NO: 88.

321. The method of any one of embodiments 318-320, wherein said vector comprises the sequence set forth as SEQ ID NO: 123.

322. The method of any one of embodiments 258-299, wherein the PAM sequence selected from the group consisting of: NNNNCC, NNGRR, and NNGG comprises said first SNP allele, and wherein said first SNP allele is a cytosine at a position corresponding to position 151 of SEQ ID NO: 1.

323. The method of embodiment 322, wherein said RGN polypeptide comprises an amino acid sequence having at least 90% sequence identity to any one of SEQ ID NOs: 3, 11, and 15.

324. The method of embodiment 322 or 323, wherein said RGN polypeptide comprises an amino acid sequence having at least 95% sequence identity to any one of SEQ ID NOs: 3, 11, and 15.

325. The method of any one of embodiments 322-324, wherein said RGN polypeptide comprises the amino acid sequence of any one of SEQ ID NOs: 3, 11, and 15.

326. The method of embodiment 322, wherein said RGN polypeptide and said guide RNA are selected from the group consisting of:
a) an RGN polypeptide comprising an amino acid sequence having at least 90% sequence identity to SEQ ID NO: 3 and a guide RNA comprising a crRNA repeat having the nucleotide sequence of SEQ ID NO: 4 or a nucleotide sequence that differs from SEQ ID NO: 4 by 1 or 2 nucleotides, and a tracrRNA having a nucleotide sequence having at least 90% sequence identity to SEQ ID NO: 5;
b) an RGN polypeptide comprising an amino acid sequence having at least 90% sequence identity to SEQ ID NO: 11 and a guide RNA comprising a crRNA repeat having the nucleotide sequence of SEQ ID NO: 12 or a nucleotide sequence that differs from SEQ ID NO: 12 by 1 or 2 nucleotides, and a tracrRNA having a nucleotide sequence having at least 90% sequence identity to SEQ ID NO: 13 or 120; and
c) an RGN polypeptide comprising an amino acid sequence having at least 90% sequence identity to SEQ ID NO: 15 and a guide RNA comprising a crRNA repeat having the nucleotide sequence of SEQ ID NO: 16 or a nucleotide sequence that differs from SEQ ID NO: 16 by 1 or 2 nucleotides, and a tracrRNA having a nucleotide sequence having at least 90% sequence identity to SEQ ID NO: 17.

327. The method of embodiment 322, wherein said RGN polypeptide and said guide RNA are selected from the group consisting of:
a) an RGN comprising an amino acid sequence having at least 95% sequence identity to SEQ ID NO: 3 and a guide RNA comprising a crRNA repeat having a nucleotide sequence that differs from SEQ ID NO: 4 by 1 nucleotide, and a tracrRNA having a nucleotide sequence having at least 95% sequence identity to SEQ ID NO: 5;
b) an RGN comprising an amino acid sequence having at least 95% sequence identity to SEQ ID NO: 11 and a guide RNA comprising a crRNA repeat having a nucleotide sequence that differs from SEQ ID NO: 12 by 1 nucleotide, and a tracrRNA having a nucleotide sequence having at least 95% sequence identity to SEQ ID NO: 13 or 120; and
c) an RGN comprising an amino acid sequence having at least 95% sequence identity to SEQ ID NO: 15 and a guide RNA comprising a crRNA repeat having a nucleotide sequence that differs from SEQ ID NO: 16 by 1 nucleotide, and a tracrRNA having a nucleotide sequence having at least 95% sequence identity to SEQ ID NO: 17.

328. The method of embodiment 322, wherein said RGN polypeptide and said guide RNA is selected from the group consisting of:
a) an RGN polypeptide comprising the amino acid sequence of SEQ ID NO: 3 and a guide RNA comprising a crRNA repeat having the nucleotide sequence of SEQ ID NO: 4 and a tracrRNA having the nucleotide sequence of SEQ ID NO: 5;
b) an RGN polypeptide comprising the amino acid sequence of SEQ ID NO: 11 and a guide RNA comprising a crRNA repeat having the nucleotide sequence of SEQ ID NO: 12 and a tracrRNA having the nucleotide sequence of SEQ ID NO: 13 or 120; and
c) an RGN polypeptide comprising the amino acid sequence of SEQ ID NO: 15 and a guide RNA comprising a crRNA repeat having the nucleotide sequence of SEQ ID NO: 16 and a tracrRNA having the nucleotide sequence of SEQ ID NO: 17.

329. The method of any one of embodiments 326-328, wherein said RGN polypeptide and said guide RNA are the RGN polypeptide and guide RNA of any one of embodiments 326(a), 327(a), or 328(a), and wherein said guide RNA comprises a spacer having a nucleotide sequence having complementarity with a target sequence of SEQ ID NO: 77 or 78.

330. The method of any one of embodiments 326-328, wherein said RGN polypeptide and said guide RNA are the RGN polypeptide and guide RNA of any one of embodiments 326(a), 327(a), or 328(a), and wherein said guide RNA comprises a spacer having the nucleotide sequence of SEQ ID NO: 82 or 83 or a nucleotide sequence that differs from SEQ ID NO: 82 or 83 by 1 or 2 nucleotides.

331. The method of any one of embodiments 326-328, wherein said RGN polypeptide and said guide RNA are the RGN polypeptide and guide RNA of any one of embodiments 326(a), 327(a), or 328(a), and wherein said guide RNA comprises a spacer having a nucleotide sequence that differs from SEQ ID NO: 82 or 83 by 2 nucleotides.

332. The method of any one of embodiments 326-328, wherein said RGN polypeptide and said guide RNA are the RGN polypeptide and guide RNA of any one of embodiments 326(a), 327(a), or 328(a), and wherein said guide RNA comprises a spacer having a nucleotide sequence that differs from SEQ ID NO: 82 or 83 by 1 nucleotide.

333. The method of any one of embodiments 326-328, wherein said RGN polypeptide and said guide RNA are the RGN polypeptide and guide RNA of any one of embodiments 326(a), 327(a), or 328(a), and wherein said guide RNA comprises a spacer having the nucleotide sequence of SEQ ID NO: 82 or 83.

334. The method of any one of embodiments 322-333, wherein said guide RNA is a single guide RNA.

335. The method of embodiment 334, wherein said RGN polypeptide and said guide RNA are the RGN polypeptide and guide RNA of any one of embodiments 316(a), 317(a), or 318(a), and wherein said single guide RNA has the nucleotide sequence of SEQ ID NO: 27 or 28.

336. The method of any one of embodiments 258-335, wherein said PAM is present only on the mutHTT allele and not the wild-type HTT allele.

337. The method of any one of embodiments 258-336, wherein said nucleic acid molecule encoding said RGN polypeptide is an mRNA.

338. The method of any one of embodiments 258-336, wherein said nucleic acid molecule encoding said RGN polypeptide and said nucleic acid molecule encoding said guide RNA are in a viral vector.

339. The method of embodiment 338, wherein said viral vector is a lentiviral vector, a baculoviral vector, or an adeno-associated viral (AAV) vector.

340. The method of embodiment 339, wherein said AAV vector is AAV5.

341. The method of any one of embodiments 258-340, wherein said nucleic acid molecule encoding said guide RNA is operably linked to an RNA polymerase III promoter.

342. The method of embodiment 341, wherein said RNA polymerase III promoter is a U6 promoter.

343. The method of embodiment 342, wherein said U6 promoter is a truncated U6 promoter.

344. The method of embodiment 343, wherein said truncated U6 promoter has the nucleotide sequence set forth as SEQ ID NO: 89 or 128.

345. The method of any one of embodiments 258-344, wherein said nucleic acid molecule encoding said RGN polypeptide is operably linked to an RNA polymerase II promoter.

346. The method of embodiment 345, wherein said RNA polymerase II promoter is a constitutive promoter.

347. The method of embodiment 346, wherein said constitutive promoter is selected from the group consisting of: a cytomegalovirus (CMV) promoter, a truncated CMV promoter, an elongation factor 1α short (EFS) promoter, and a JeT promoter.

348. The method of embodiment 347, wherein said constitutive promoter is a JeT promoter.

349. The method of embodiment 348, wherein said JeT promoter has the nucleotide sequence set forth as SEQ ID NO: 92.

350. The method of embodiment 345, wherein said RNA polymerase II promoter is a tissue-specific promoter.

351. The method of embodiment 350, wherein said tissue-specific promoter is a brain or neuron specific promoter.

352. The method of embodiment 351, wherein said brain or neuron specific promoter is selected from the group consisting of: a human synapsin I (Syn) promoter, a 67 kDa glutamic acid decarboxylase (GAD67) promoter, a 65 kDa glutamic acid decarboxylase (GAD65) promoter, a homeobox Dlx5/6 promoter, a preprotachykinin 1 (Tac1) promoter, a neuron-specific enolase (NSE) promoter, a dopaminergic receptor 1 (Drd1a) promoter, a dopaminergic receptor 2 (DRD2) promoter, and glial fibrillary acidic protein (GFAP) promoter.

353. The method of embodiment 352, wherein said neuron specific promoter is a Syn promoter.

354. The method of embodiment 353, wherein said Syn promoter has the nucleotide sequence set forth as SEQ ID NO: 93.

355. The method of any one of embodiments 258-354, wherein said nucleic acid molecule encoding said RGN comprises a polyadenylation (polyA) tail.

356. The method of embodiment 355, wherein said polyA tail is a SV40 polyA tail or a bovine growth hormone (bGH) polyA tail.

357. The method of embodiment 356, wherein said SV40 polyA tail has the nucleotide sequence set forth as SEQ ID NO: 94.

358. The method of embodiment 356, wherein said bGH polyA tail has the sequence set forth as SEQ ID NO: 95.

359. The method of any one of embodiments 258-358, wherein said RGN is operably linked to at least one nuclear localization signal.

360. The method of embodiment 359, wherein said at least one nuclear localization signal comprises an SV40 nuclear localization signal.

361. The method of embodiment 360, wherein said SV40 nuclear localization signal has the sequence set forth as SEQ ID NO: 86.

362. The method of embodiment 359, wherein said at least one nuclear localization signal comprises a c-Myc nuclear localization signal.

363. The method of embodiment 362, wherein said c-Myc nuclear localization signal has the sequence set forth as SEQ ID NO: 125.

364. The method of any one of embodiments 359-363, wherein a NLS linker protein connects said RGN polypeptide and said at least one nuclear localization signal.

365. The method of embodiment 364, wherein said NLS linker protein has the sequence set forth as SEQ ID NO: 127.

366. A method for detecting mutant huntingtin (mutHTT) protein and wild type HTT (wtHTT) protein in a sample, said method comprising:
a) applying a sample that has been denatured to a capillary comprising a sieving medium;
b) applying a voltage differential to said capillary to separate proteins within said sample by molecular weight via electrophoresis;
c) immobilizing said separated proteins within said capillary;
d) applying to said capillary a first antibody or fragment thereof capable of binding to both mutHTT and wtHTT;
e) applying to said capillary a second antibody or fragment thereof capable of binding to said first antibody, wherein said second antibody comprises a detectable label; and
f) detecting said detectable label.

367. The method of embodiment 356, wherein said sieving medium is a hydrophilic polymer matrix.

368. The method of embodiment 366 or 367, wherein said sample is a biological sample.

369. The method any one of embodiments 366-368, wherein said detectable label is a chemiluminescent label or a fluorescent label.

370. The method of any one of embodiments 366-369, wherein said mutHTT and wtHTT protein in said sample are quantitated by comparison to a standard curve.

371. The method of any one of embodiments 366-370, wherein said method is capable of resolving the mutHTT protein from the wtHTT.

372. A method of ameliorating or delaying the onset of one or more symptoms of Huntington's disease (HD) in a subject in need thereof, wherein said method comprises delivering to said subject an adeno-associated viral (AAV) 5 vector comprising:
a) a guide RNA having a crRNA of SEQ ID NO: 8 or 106 and a tracrRNA of SEQ ID NO: 9 or 107; or
b) a single guide RNA having the nucleotide sequence of SEQ ID NO: 25 or 26.

373. The method of embodiment 372, wherein the subject comprises a mutant huntingtin (mutHTT) allele comprising:
a) at least 36 CAG repeats in exon 1; and
b) a first single nucleotide polymorphism (SNP) allele in exon 50, wherein said SNP allele comprises a thymine at a position corresponding to position 151 of SEQ ID NO: 1.

374. The method of embodiment 372 or 373, wherein said method comprises administering the AAV5 vector by intrastriatal injection.

375. The method of any of embodiments 372-374, wherein said subject has a decrease in a level of mutHTT protein encoded by said mutHTT allele as compared to a leve of HTT protein of a control subject or a level of wild type HTT protein.

376. The method of embodiment 375, wherein at least a 40% decrease in the level of mutHTT protein is observed as compared to a level of HTT protein of a control subject or a level of wild type HTT protein.

377. The method of embodiment 375 or 376, wherein a decrease in the level of mutHTT protein is observed by 4 weeks, 6 weeks, 8 weeks, 10 weeks, or 12 weeks after administration of said vector.

378. The method of any of embodiments 375-377, wherein a decrease in the level of mutHTT protein is observed in at least 50% of the striatal cells in said subject.

379. The method of any one of embodiments 372-378, wherein a level of mutHTT mRNA is reduced by at least 40% as compared to a level of HTT mRNA in a control subject or a level of wild type HTT mRNA.

380. The method of any one of embodiments 372-379, wherein said mutHTT allele has at least 40 CAG repeats in exon 1.

381. The method of any one of embodiments 372-379, wherein said mutHTT allele has at least 56 CAG repeats in exon 1 and wherein said subject is younger than 18 years of age.

382. The method of any one of embodiments 372-381, wherein said administering occurs prior to onset of symptoms of Huntington's disease.

383. The method of any one of embodiments 372-382, wherein said method comprises preventing the onset of one or more symptoms of Huntington's disease.

384. The method of any one of embodiments 372-383, wherein said subject has at least one symptom of Huntington's disease.

385. The method of any of embodiments 372-384, wherein only the mutant HTT allele is edited.

The following examples are offered by way of illustration and not by way of limitation.

### EXAMPLES

### Example 1. Testing of Guide RNAs and Associated RNA-Guided Nucleases (RGNs) in Patient Fibroblasts and Target Shortening

### Identification of Single Nucleotide Polymorphisms (SNPs) for Allele-Specific Targeting of Mutant HTT

Using the Mar. 29, 2022 version of NCBI dbSNP, all single nucleotide polymorphisms (SNPs) in the human huntingtin (HTT) gene with a minor allele frequency (MAF) between 0.05 and 1.0 were analyzed for their ability to be used for allele-specific targeting with RNA guided nucleases. Only SNPs in the coding regions of the HTT gene were considered. The location of the SNP in the “Region” column in Tables 1 and 2 has been underlined. The “pos” column in Table 1 lists the location of the SNP in chromosome 4 of the reference human genome hg38 build (NCBI Assembly No. GRCh38.p14).

TABLE 1
SNPs in HTT with MAF >0.05 in coding regions

| | | | Variant | | | |
|---|---|---|---|---|---|---|
| SNP ID | Position | Variation | type | Function | Frequency | Region |
| 362272 | 3233253 | G > A, T | snv | missense_variant | A:0.210264:1053:1000Genomes | GCGTC |
| 362273 | 3225692 | A > G, T | snv | synonymous_variant | G:0.246406:1234:1000Genomes | CTACA |
| 362331 | 3214108 | T > C | snv | missense_variant | C:0.441294:2210:1000 Genomes | TCTAC |
| 362336 | 3212105 | G > A | snv | synonymous_variant | A:0.239617:1200:1000Genomes | GAGCC |
| 363099 | 3160329 | C > A, T | snv | synonymous_variant | T:0.213858:1071:1000 Genomes | CTCCG |
| 363125 | 3187820 | C > A | snv | missense_variant | A:0.189097:947:1000 Genomes | TACTT |
| 1065745 | 3121347 | C > T | snv | synonymous_variant | T:0.072284:362:1000 Genomes | ACCGC |
| 2276881 | 3229934 | G > A | snv | synonymous_variant | A:0.144569:724:1000 Genomes | CTGAT |

A bioinformatic analysis was performed to determine if the major or minor allele could be used to selectively target one allele in a heterozygous individual. The following RGNS and PAMs were considered: APG07433.1 with an NNNTNCC PAM, APG05586 with an NNRYA PAM, APG01604 with an NNGRR PAM, and LPG10145 with an NNGG PAM. The allele frequency in a Huntington patient database (GemHD) was also considered.

TABLE 2
HTT SNPs with allele-specific targeting potential

| | | | Major | Minor | MAF in 1000 | MAF in |
|---|---|---|---|---|---|---|
| SNP ID | Variation | Region | Allele | Allele | Genomes | Gem HD |
| 362272 | G > A, T | GCGT | — | APG01604 | A: 0.210264 | A: 0.173 |
| | | C | | [A] | | |
| 362331 | T > C | TCTA | APG05586 | APG07433.1, | C: 0. 441294 | C: 0.256 |
| | | C | [T] | APG01604, | | |
| | | | | LPG10145 | | |
| | | | | [C] | | |
| 363099 | C > A, T | CTCC | APG07433.1, | APG01604, | T: 0.213858; | T: 0.172 |
| | | G | LPG10145 | APG05586 | A: ? | |
| | | | [C] | [A] | | |
| 363125 | C > A | TACTT | APG05586 | — | A: 0.189097 | A: 0.078 |
| | | | [C] | | | |
| 1065745 | C > T | ACCG | APG07433.1, | — | T: 0.072284 | T: 0.036 |
| | | C | LPG10145, | | | |
| | | | APG05586 | | | |
| | | | [C] | | | |

### Phasing of HD Patient Fibroblast Cell Lines

Huntington's Disease is caused by an expanded CAG trinucleotide repeat in the huntingtin gene (HTT). HD is an autosomal dominant disease, but because the wild-type allele encoding the normal HTT protein is required for normal neurological function, there is currently unmet need for safe therapeutic strategies that selectively target and inactivate the mutant HTT allele and associated mutant HTT protein. The HTT exon 50 SNP rs362331 results in a PAM site which can be used for allele selective targeting based on the presence of a cytosine (using APG07433.1) or thymine (using APG05586) nucleotide. Crucially, while the presence of the SNP generates the opportunity for allele selective editing, the CAG repeat expansion can exist on either allele, thus, to selectively target the mutant allele and associated protein, the CAG repeat expansion must be located on (in-phase with) the targeted allele. For example, to selectively target the mutant allele with APG05586, the CAG repeat expansion must be present on the ‘T’ allele. Accordingly, to selectively target the mutant allele with APG07433.1, the CAG repeat expansion must be present on the “C” allele. Several cell lines were tested to determine the number of CAG repeats and identify if the major or minor allele for rs362331 were in phase with the CAG repeat expansion.

### Initial Genotyping for SNPs

Genomic DNA was extracted from 1e6 cells and 20 ng-80 ng of gDNA was used for subsequent assays. Polymerase chain reaction (PCR) was run using SNP specific primers (Table 4) at 2 M with Platinum SuperFI II taq polymerase (ThermoFisher) according to manufacturer's instructions.

Cycling conditions are shown in Table 3.

TABLE 3
Cycling conditions for genotyping.

| | | | |
|---|---|---|---|
| Cycle step | Temperature | Time | Cycles |

| | | | | | |
|---|---|---|---|---|---|
| Initial denaturation | 98° | C. | 30 | seconds | 1 |
| Denaturation | 98° | C. | 10 | seconds | 35 |
| Annealing | 60° | C. | 10 | seconds | |
| Extension | 72° | C. | 15 | seconds | |
| Final extension | 72° | C. | 5 | minutes | 1 |

| | | | |
|---|---|---|---|
| End | 4° | C. | Hold |

Amplicons were visualized via LONZA flash gel run at 225V for 5 mins. PCR products were then subsequently purified and concentrated and sequenced along with the corresponding FWD and REV primers at 10 μM. SNPs were determined by visualization of the chromatogram obtained from Sanger sequencing results.

TABLE 4
SNP Specific Primer Sequences for PCR

| | FWD primer | REV primer |
|---|---|---|
| SNP ID | (SEQ ID NO: 19) | (SEQ ID NO: 20) |
| 362331 | AAACGAAGGTACACGAGTGG | GCAAACAGGCAGCACAAAAT |

TABLE 5
Phasing of CAG repeats and rs362331.

| Cell name | Genotype/phasing for rs362331 | CAG repeats |
|---|---|---|
| GM08399 WT | C/C | N/A |
| HEK293 | T/T | N/A |
| GM04869 HD | T/T | 49/17 |
| GM04281 jHD | T/T | 78/17* |
| GM09197 jHD | T > C | 180/18* |
| GM02173 HD | T > C | 45/19 |
| ND29970 HD | C/C | 41/19 |
| ND31551 HD | C > T | 39/18 |
| ND30259 HD | C > T | 40/23 |
| ND30260 HD | C > T | 40/19 |
| GM04723 | C/T | 17/69 |
| GM21756 | C/T | 17/69 |

*results from Chao, M. et al., 2017, European Journal of Human Genetics doi.org/10.1038/ejhg.2017.125

To determine the phasing of the CAG expansion, a modified method and primer sequences were used from Liu et. al., 2008, Nat. Meth. 5(11):951-953. To amplify the CAG repeats, a 50 μL long-range PCR was performed via LA-taq DNA polymerase using GC buffer I.

For this first reaction, primers (10 uM) with KasI restriction sites were as follows:

Forward Exon 1-
(SEQ ID NO: 21)
5′- AGCTGATGGGCGCCTTCGAGTCCCTCAAGTC-3′,
Reverse Exon 50-
(SEQ ID NO: 22)
5′-TGCACTGCAGGCGCCTCCAGGATGAAGTGCACAC -3′.

Thermocycling conditions are shown in Table 6.

TABLE 6
Thermocycling conditions for amplifying CAG repeats.

| | | | |
|---|---|---|---|
| Cycle step | Temperature | Time | Cycles |

| | | | | | |
|---|---|---|---|---|---|
| Initial denaturation | 98° | C. | 3 | mins | 1 |
| Denaturation | 98° | C. | 30 | seconds | 32 |
| Annealing | 55° | C. | 30 | seconds | |
| Extension | 72° | C. | 2 | mins | |
| Final extension | 72° | C. | 5 | minutes | 1 |

| | | | |
|---|---|---|---|
| End | 4° | C. | Hold |

PCR products were analyzed by electrophoresis on an 1% (w/v) agarose Tris-Acetate EDTA (TAE) gel containing 1× gel green using a 1 kb plus ladder (New England Biolabs). The correctly sized PCR bands were excised, purified, and subsequently used for the inverse PCR.

The purified-PCR products were digested with 10 U of KasI in rCutSmart Buffer (New England Biolabs) in a 20 μL total reaction at 37° C. for 1h. Digested DNA was purified, concentrated, and eluted in a volume of 10 μL.

The product was then circularized in a 20 μL reaction and incubated at room temperature for 15 mins. Ligated DNA was then cleaned and concentrated and eluted in a volume of 10p L.

Products were then digested in a reaction volume of 50 μL with 5U of Exonuclease V with ATP in NEBuffer 4. The reaction was incubated in a thermocycler under the following conditions: 37° C. for 30 mins, 70° C. for 30 mins to deactivate, then held at 12° C. Digested DNA was then concentrated and cleaned as stated above and then directly used for the inverse PCR.

The inverse PCR was ran using SuperFi II platinum taq (ThermoFisher) with the following primers at 2 μM:

Inverse Forward Exon 50-
(SEQ ID NO: 23)
5′- CAGATCCCGCTGAGTCTGGATCTCC -3′
Inverse Reverse Exon 1-
(SEQ ID NO: 24)
5′- CGGCTGAGGCAGCAGCGGCT -3′

Cycling conditions are listed in Table 6.

Bands were analyzed using electrophoresis as stated above with the selected bands purified and concentrated. Extracted DNA was then submitted for sanger sequencing. Phasing was determined by the number of CAG repeats and visualization of the chromatogram at the rs362331 SNP.

Occasionally, the correct phasing of a cell line was unable to be identified due to the inability to differentiate alleles with smaller CAG repeats. To overcome this, the final inverse PCR product was cloned. A total of 10 colonies per cell line were picked and grown up in 5 mL of LB with Kanamycin in a shaking incubator set at 37° C. overnight. Subsequently, the cells were processed and purified DNA was then submitted to sanger sequencing using the standard M13 reverse and forward primers. Phasing was determined from the Sanger sequencing results as stated above.

Screening Guide RNAs to Selectively Target the C or T Allele of rs362331

Fibroblasts were maintained at 37° C., 95% humidity, and 5% CO₂ in FB media composed of: 1×MEM, 15% FBS, 1× Non-essential amino acids, 1×GlutaMax and 1% Pen/strep (ThermoFisher). Once the cells reached an 80-90% confluency, fibroblasts were typsinized with trypLE (ThermoFisher) at 37° C. for 5 mins or until cells detached from the surface as observed by the microscope. Once 80-90% of cells detached, TrypLE was inactivated with 3× volume of FB media. Cells were centrifuged at 90×g for 10 mins at room temperature and rinsed with pre-warmed 1× dulbecco's phosphate-buffered saline (DPBS, ThermoFisher). Cells were centrifuged again at 90×g for 10 mins, then split at a 1 to 3 ratio. All cell lines were subjected to sterility and mycoplasma testing via IDEXX Bioanalytics with 1 million cells.

Guides were synthesized with phosphorothioated 2′-O-methyl on the first and last three RNA bases to prevent degradation. Lyophilized guide RNAs were resuspended at 2 μg/uL in sterile TE buffer in a sterile environment.

Fibroblasts were trypsinized as stated above and a total cell count was determined by a 1 to 1 ratio of cells to Trypan Blue 0.04% and an automated cell counter. Guide RNA (4 μg) and nuclease-expressing mRNA (2 μg) were nucleofected in a total of 200,000 live cells per reaction with 20 μL of P2 buffer (LONZA). Guides were screened in various genotypes at the specific rs362331 SNP, either homozygous for the C, heterozyous C/T, or homozygous for T. Cells were then incubated at room temperature for 10mins then resuspended with 80 μL of prewarmed FB media, then transferred to a 24-well plate containing 1 mL of FB media.

Twenty-four hours after plating, media was exchanged. Four days post nucleofection, cells were harvested for gDNA, protein, and mRNA for downstream analysis of INDELs, capillary western, or RTq-PCR/ddPCR respectively (see Table 8).

mRNA quantification through RT-qPCR digital PCR (dPCR)

Total RNA from cell pellets were extracted and measured via nanodrop and 1 μg of RNA was treated with DNase I (Invitrogen). Complementary DNA (cDNA) was converted using 1 U of SuperScript IV (Invitrogen) with 100-500 ng of RNA using Oligo d(T)20 primers and ribonuclease inhibitor following manufacturer's instructions. cDNA was subsequently treated with RNase H (Invitrogen) to ensure a pure yield of cDNA. Taqman probes spanning exons 64-65 and 10-11 were used to detect HTT mRNA (ThermoFisher). Taqman probes targeting GAPDH was used as housekeeping gene (Thermofisher). 10 ng of cDNA was used per 10 μL reaction with 1× Taqman Fast Advanced Mastermix (ThermoFisher) and run on QuantStudio 3 Real-Time PCR. Each sample was run in technical duplicates.

TABLE 7
Thermocycling conditions for amplifying HTT mRNA

| | | |
|---|---|---|
| Temperature | Time | Cycles |

| | | | |
|---|---|---|---|
| 50 C. hold | 2 | min | 1 |
| 95 C. hold | 2 | min | 1 |
| 95 C. | 1 | sec | 40 |
| 60 C. | 20 | sec | |

Quantification of fold change was determined by a fold change to GAPDH, then to nuclease only (see Tables 16 and 17).

### Protein Quantification JESS

To obtain total protein, cell pellets were lysed via water bath sonication for 10s and centrifuged at 1000 RPM for 5 mins. The supernatant was collected and total protein concentration was determined. Protein was diluted to a 0.5 mg/mL concentration with 0.1× sample buffer (ProteinSimple). Antibodies were diluted in the provided antibody diluent. Antibodies against total HTT protein (MAB2166, Millipore, and EPR5526, Abcam) were used at a 1:50 dilution with the appropriate secondary. Antibody detecting polyQ disease proteins (clone 3B5H10; Miller, J et. al., 2011, Nat. Chem. Bio. 7(12):925-934) was used at a 1:30 dilution with the corresponding secondary. Anti-Vinculin (VCL; Abnova) was used as a loading control at a 1:50 dilution. Samples and plate were prepared following manufacturer's instructions. Due to the nature of the 180 polyQ repeats in cell line GM09197, we were able to separate the mutant HTT protein from the wildtype HTT protein. Areas of the mutant and/or wildtype HTT protein peaks were normalized to VCL then nuclease alone (see Table 14).

### Indel Methods

Post-transfection of guide and nuclease, the total genomic DNA was then analyzed to determine the rate of editing for each RGN for each genomic target. First, oligonucleotides were produced to be used for PCR amplification and subsequent analysis of the amplified genomic target site. Large genomic regions encompassing each target gene were first amplified using PCR #1 primers, using a program of: 98° C., 1 min; 30 cycles of [98° C., 10 sec; 62° C., 15 sec; 72° C., 5 min]; 72° C., 5 min; 12° C., forever. One microliter of this PCR reaction was then further amplified using primers specific for each guide (PCR #2 primers), using a program of: 98° C., 1 min; 35 cycles of [98° C., 10 sec; 67° C., 15 sec; 72° C., 30 sec]; 72° C., 5 min; 12° C., forever. Primers for PCR #2 include Nextera Read 1 and Read 2 Transposase Adapter overhang sequences for Illumina sequencing. Following the second PCR amplification, DNA was cleaned and eluted in water. 200-500 ng of purified PCR #2 product was combined with 2 μL of 10×NEB Buffer 2 and water in a 20 μL reaction and annealed to form heteroduplex DNA using a program of: 95° C., 5 min; 95-85° C., cooled at a rate of 2° C./sec; 85-25° C., cooled at a rate of 0.1° C./sec.; 12° C., forever. Following annealing 5 μL of DNA was removed as a no enzyme control, and 1 μL of T7 Endonuclease I (NEB) was added and the reaction incubated at 37° C. for 1 hr. After incubation 5× FlashGel loading dye (Lonza) was added and 5 μL of each reaction and controls were analyzed by a 2.2% agarose FlashGel (Lonza) using gel electrophoresis.

Additionally, products from PCR #2 containing Illumina overhang sequences underwent library preparation following the Illumina 16S Metagenomic Sequencing Library protocol. Deep sequencing was performed on an Illumina Mi-Seq platform by a service provider (MOGene). Typically, 200,000 of 250 bp paired-end reads (2×100,000 reads) are generated per amplicon. The reads were analyzed using CRISPResso (Pinello, et al. 2016 Nature Biotech, 34:695-697) to calculate the rates of editing. The editing rate is reported as the number of reads with indels (insertion and deletion) divided by total reads.

### Recombinant AAV Production Methods

For AAV expression cassette generation, plasmids were synthesized using the cloning vector (pUC57) as a plasmid backbone for insertion of transgenes and AAV2 inverted terminal repeats (ITRs). The full nucleotide composition of all AAV plasmids were verified using Sanger sequencing analysis following synthesis and amplification. Recombinant AAV vectors carrying RGN and concordant guide transgenes were generated internally. To generate AAV vectors, the triple-transfection plasmid protocol of production was used as described previously (Grieger & Choi, & Samulski. 2006, Nature Protocols 1(3):1412-1428). Briefly, expression plasmid carrying transgene sequences were supplemented with a helper plasmid and a packaging plasmid via de novo plasmid synthesis which contains a chimera of AAV2 and AAV5 rep genes along with the AAV5 capsid gene.

The plasmids were transfected into 293T cells (ATCC® CRL-3216™), and replicated viral vectors were collected and purified by cesium chloride (CsCl) gradient. The purified recombinant vectors were subjected to diafiltration, followed by assessment of: 1) physical titer by digital droplet PCR and ELISA; and 2) quantification of ratios of viral proteins 1, 2, and 3 by antibody based capillary electrophoresis.

### Delivery Methods and Summary of Results

APG05586 guide RNAs of different backbone length resulted in similar indel formation rates across patient fibroblasts (Table 8). SGN004282 had a 76 bp backbone and SGN004281 had a 90 bp backbone.

TABLE 8
Comparison of INDEL formation by APG05586 guide RNAs with differing backbone length.

| % INDELS by cell | | | | | | | | | |
|---|---|---|---|---|---|---|---|---|---|
| line Technical | GM08339 | | | | | | | | |
| replicates | rs362331 C/C | | | GM09197 rs362331 C/T | | | GM04281 rs362231 T/T | | |
| SGN004281 (SEQ ID | 0.152859 | 0 | 0 | 34.95471 | 33.88744 | 31.27415 | 60.6322 | ND | 79.07996 |
| NO: 25) (APG05586) | | | | | | | | | |
| SGN004282 (SEQ ID | 0 | 0 | 0 | 42.82928 | 37.32604 | 25.01887 | 72.525 | 57.27451 | 76.66433 |
| NO: 26) (APG05586) | | | | | | | | | |

ND = not determined

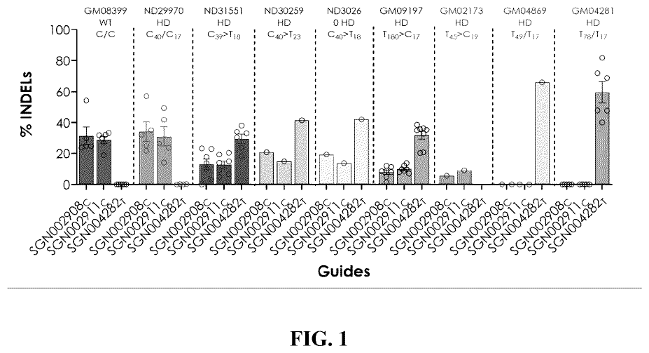

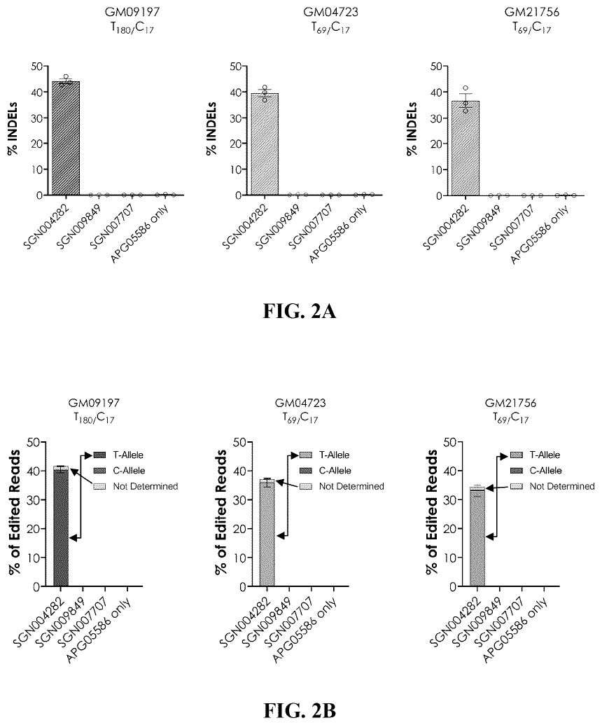

As shown in Tables 9 and 10 and FIGS. 1,2A, and 2B, the 22 nt-spacer guide (SGN002911) targeting the C-allele of rs362331 showed on average an INDEL % of 29.87 and 11.52 in homozygous and heterozygous C lines respectively. Similarly, the 25-nt spacer guide (SGN002908) exhibited an average of INDEL % of 32.53 and 11.21 in homozygous and heterozygous C lines respectively. Both guides SGN002911 and SGN002908 produced a minimal amount of INDELs in homozygous T cell lines: 0.04% and 0.0%, respectively. The 25 nt-spacer guide with a 76 nt backbone targeting the T-allele of rs362331, SGN004282, showed 60.52% average INDEL across homozygous T lines, 32.06% across heterozygous T lines, and 0.08% in homozygous C lines. These INDEL results suggest that all the guides targeting either the C- or T-allele in patient fibroblasts are effective at creating insertions and deletions at the targeted gene.

TABLE 9
INDEL formation by APG07433.1 guide RNAs and an APG05586 guide RNA.

| | | SGN002908C | SGN002911C | SGN004282T |
|---|---|---|---|---|
| % INDELs by guide | | (SEQ ID NO: 27) | (SEQ ID NO: 28) | (SEQ ID NO: 26) |
| and cell line | | (APG07433.1) | (APG07433.1) | (APG05586) |
| GM08399 WT | C/C | ND | 30.20188368 | 0 |
| | | 24.56576068 | 18.72809214 | 0 |
| | | 29.95 | 33.36 | 0.06 |
| | | 23.58 | 32.28 | 0.06 |
| | | 24.62 | 26.83 | 0 |
| | | 54.36292 | 32.00078 | 0 |
| GM09197 HD | T180 > C17 | 12.09531516 | 13.78108024 | 38.65907927 |
| | | ND | ND | 35.05806 |
| | | 4.808136313 | 7.909362375 | 35.38040816 |
| | | 11.27069413 | 9.781442503 | 36.49997708 |
| | | 8.46 | 12.23 | 32.91 |
| | | 8.92 | 9.89 | 20.62 |
| | | 7.89 | 8.62 | 20.17 |
| | | 1.229501 | 6.936684 | 35.75073 |
| ND29970 HD | C40/C18 | 24.96181643 | 23.49842999 | 0 |
| | | 21.60095608 | 14.0119663 | ND |
| | | 32.32235998 | 49.32306876 | 0 |
| | | 35.24134097 | 26.4669579 | 0.325895924 |
| | | 57.07 | 41.82 | 0.32 |
| ND31551 HD | C39 > T18 | 22.79153601 | 20.38164513 | 33.13823592 |
| | | 3.140967031 | 4.891017651 | ND |
| | | 9.795329871 | 9.200204464 | 30.60775518 |
| | | 17.71760535 | 13.92857208 | 34.16458583 |
| | | 23.11 | 19.18 | 38.19 |
| | | 13.63 | 14.87 | 23.51 |
| | | 0 | 6.41925 | 18.18505 |
| GM02173 | T45 > C19 | 5.642846538 | 9.10715733 | ND |
| GM04869 | T49/T17 | 0.00 | 0 | ND |
| | | 0.00 | 0.1 | 66.04274373 |
| GM04281 | T78/T78 | ND | ND | 68.82128 |
| | | 0 | 0.088271735 | 81.66562368 |
| | | 0 | 0.09 | 47.58 |
| | | 0 | 0 | 39.96 |
| | | 0 | 0 | 47.7 |
| | | 0 | 0 | 71.84655 |
| ND30259 | C40 > T23 | 20.76 | 14.91 | 41.44 |
| ND30260 | C40 > T18 | 19.28 | 13.73 | 42.06 |

ND = not determined

TABLE 10
INDEL formation by APG05586 guide RNA.

| | | SGN009849 | SGN007707 | SGN004282T |
|---|---|---|---|---|
| | | (SEQ ID | (SEQ ID | (SEQ ID |
| % INDELs by guide | | NO: 129) | NO: 131) | NO: 26) |
| and cell line | | (APG05586) | (APG05586) | (APG05586) |
| GM09197 | C17/T180 | 0 | 0 | 43.57 |
| | | 0.08 | 0 | 46.03 |
| | | 0 | 0 | 42.74 |
| GM04723 | C17/T69 | 0 | 0 | 41.87 |
| | | 0.2 | 0 | 39.84 |
| | | 0.1 | 0 | 36.87 |
| GM21756 | C17/T69 | 0 | 0.03 | 32.76 |
| | | 0.11 | 0 | 41.6 |
| | | 0.11 | 0 | 35.72 |

Initial screening of guide SGN004333 for APG01604 targeting the C-allele at rs362331 generated very little IN4DELs, suggesting an inefficient guide and nuclease combination for on-target editing (Table 11).

TABLE 11
INDEL formation by APG01604 guide RNA

| % INDELS by cell | | | | | | | | | |
|---|---|---|---|---|---|---|---|---|---|
| line Technical | | | | GM09197 | | | GM04281 | | |
| replicates | GM08339 rs362331 C/C | | | rs362331 C/T | | | rs362231 T/T | | |
| SGN004333 (SEQ | 0.174584 | 0 | 0.305613 | 0 | 0 | 0 | 0 | 0 | 0 |
| ID NO: 29) | | | | | | | | | |
| APG01604 only | 0.078486 | 0.041195 | ND | 0 | 0 | ND | 0 | ND | ND |

ND = not determined

HD patient fibroblasts were transduced with an AAV6 (Table 12) or AAV2 (Table 13) generated from the construct set forth as SEQ ID NO: 32 at various MOIs for seven days. The construct comprises pAAV-hU6(318 bp)-SGN004282-JeT-APG05586-SV4 pA(179 bp). While these constructs contain E. coli codons, it is surprising that neither AAV2- nor AAV6-pAAV-hU6(318 bp)-SGN004282-JeT-APG05586-SV40pA(179 bp) were able to produce meaningful IN4DELs in patient fibroblasts.

TABLE 12
INDEL formation by AAV6- pAAV-hU6(318 bp)-
SGN004282-JeT-APG05586-SV40pA(179 bp).

| | Desired MOI(AAV6) | |
|---|---|---|
| Cell line | APG05586 + SGN004282 | INDELs |
| GM09197 | 1500000 | 1.63 |
| GM09197 | 1000000 | 0.62 |
| GM09197 | 300000 | 0 |
| GM09197 | 100000 | 0 |
| GM09197 | 30000 | 0 |
| GM09197 | Vehicle | 0 |
| GM09197 | Naive | 0 |
| GM04281 | 1500000 | 2.19 |
| GM04281 | 1000000 | 1.31 |
| GM04281 | 300000 | 1.38 |
| GM04281 | 100000 | 0 |
| GM04281 | 30000 | 0 |
| GM04281 | Vehicle | 0 |
| GM04281 | Naive | 0 |

TABLE 13
INDEL formation by AAV2- pAAV-hU6(318 bp)-
SGN004282-JeT-APG05586-SV40pA(179 bp)

| | | Percent |
|---|---|---|
| Experimental Condition | MOI | Indels |
| Naive | — | 0 |
| AAV2 (APG05586 + SGN004282) | 30000 | 0 |
| AAV2 (APG05586 + SGN004282) | 100000 | 0 |
| AAV2 (APG05586 + SGN004282) | 300000 | 0 |
| AAV2 (APG05586 + SGN004282) | 1000000 | 0 |
| AAV2 (APG05586 + SGN004282) | 1500000 | 0 |
| Naive | — | 0 |

As shown in Table 14, AAV6 vectors carrying constructs containing a mammalian codon-optimized APG05586 sequence are capable of transducing, expressing, and editing rs362331 SNP target in HD patient fibroblast line GM04281. The IN4DEL rates show a dose-dependent response.

TABLE 14
INDEL formation rates in HD patient fibroblasts
transduced with AAV6 comprising APG05586.

| AAV test article in GM04281 | | |
|---|---|---|
| (T/T) | MOI | INDELS |
| pAAV-hU6-SGN004282-Jet- | 100000 | 0.09 |
| APG05586op-SV40pA(179 bp) | 250000 | 3.76 |
| (PR_073) | 500000 | 6.91 |
| (SEQ ID NO: 34) | 1000000 | 4.29 |
| pAAV-hU6-SGN004282-CMVeb- | 100000 | 1.6 |
| APG05586op-SV40pA (PR_073) | 250000 | 6.45 |
| (SEQ ID NO: 36) | | |
| pAAV-hU6-SGN004282-EFS- | 100000 | 0 |
| APG05586op-SV40pA (PR_073) | 250000 | 1.14 |
| (SEQ ID NO: 37) | 500000 | 1.12 |
| | 1000000 | 0.86 |
| pAAV-hU6(241 bp)-SGN004282- | 100000 | 0 |
| CMVeb-APG05586op- | 250000 | 1.06 |
| bGHpA(225 bp)(PR_073) | 500000 | 4.65 |
| (SEQ ID NO: 38) | 1000000 | 12.07 |
| pAAV-hU6(241 bp)-SGN004282-EFS- | 100000 | 0.18 |
| APG05586op-bGHpA(225 bp) | 250000 | 0.42 |
| (PR_073)(SEQ ID NO: 39) | 500000 | 1.73 |

Homozygous C or T and heterozygous fibroblast cell lines were edited with a C-guide (SGN002908 and SGN002911) or T-guide (SGN004282). Due to the nature of polyQs in GM09197, the difference between WT and mutant HTT protein was able to be resolved. Chao, M J, et. al., 2017, European Journal of Human Genetics 25:1202-1209 showed that mutant HTT protein was linked to the T-allele and WT to the C-allele in GM09197. As shown in Tables 15 and 16, when testing allele-specific guides, the C-guides, SGN002908 and SGN002911 were able to reduce WT HTT protein by ˜0.40% and ˜0.30% o, respectively, without affecting the mutant HTT protein. On the other hand, SGN004282 which targets the T-allele, reduced the mutant HTT protein by ˜0.56% o without affecting the WT HTT protein (Tables 15 and 16). Thus, the constructs tested herein demonstrate selectivity at the protein level in addition to showing allele selective IN4DELs in the HTT gene.

TABLE 15
Mutant HTT protein in patient fibroblasts normalized
to vinculin, then corresponding RGN.

GM09197 HD
T₁₈₀/C₁₇

| | | | |
|---|---|---|---|
| SGN002908_{C} | 1.255103 | ND | ND |
| SGN002911_{C} | 1.121519 | ND | ND |
| APG07433.1 | 1 | ND | ND |
| only | | | |
| SGN004282_{T} | 0.632687 | 0.253607 | 0.453947 |
| APG05586 only | 1 | 1 | 1 |

ND = not determined

TABLE 16
Wild type HTT protein in patient fibroblasts normalized to vinculin, then corresponding RGN.

| | GM08399 WT C/C | | | GM09197 HD T₁₈₀ > C₁₇ | | GM04281 T₇₈/T₁₇ |
|---|---|---|---|---|---|---|
| SGN002908_{C} | 0.74094 | 0.851727 | ND | 0.776972 | 0.438663 | 1.208498 |
| SGN002911_{C} | 0.79706 | 1.206674 | ND | 0.871639 | 0.567172 | 1.247716 |
| APG07433.1 only | 1 | 1 | ND | 1 | 1 | 1 |
| SGN004282_{T} | 1.030307 | 1.022099 | 1.08 | 0.983985 | 0.914516 | 0.754316 |
| APG05586 only | 1 | 1 | 1 | 1 | 1 | 1 |

ND = not determined

As shown in Tables 17 and 18, mRNA from edited patient fibroblasts exhibited a decrease in HTT mRNA at both the exon 64-65 and exon 10-11 junction. The C-guides SGN002908 and SGN002911 decreased HTT mRNA in a homozygous C fibroblast cell line by ˜30% and 20%, respectively. The T-guide SGN004282 decreased HTT mRNA in a homozygous T fibroblast cell line by ˜50%, and in heterozygous C/T fibroblast cell line by ˜30%. Despite successfully reducing HTT mRNA in a homozygous C fibroblast line, the C-guides were unable to reduce HTT mRNA in a heterozygous cell line. This may be due to the less efficient activity of the C-guides relative to the T-guides, and the fact that in a heterozygous cell line there is only one applicable genomic target.

TABLE 17
HTT mRNA levels at exon 64-65 with C-guides or T-guides.

| mRNA Fold change | | | | | | |
|---|---|---|---|---|---|---|
| Exon 64-65 | GM08399 WT C/C | | GM09197 HD T₁₈₀ > C₁₇ | | GM04281 T₇₈/T₁₇ | |
| SGN002908_{C} | 0.7 | ND | 1.05 | ND | 1.05 | ND |
| SGN002911_{C} | 0.76 | ND | 1.03 | ND | 0.95 | ND |
| SGN004282_{T} | 1.05 | 0.97 | 0.79 | 0.71 | 0.6 | 0.37 |

ND = not determined

TABLE 18
HTT mRNA levels at exon 10-11 with C-guides or T-guides.

| mRNA fold change | | | | | | |
|---|---|---|---|---|---|---|
| Exon 10-11 | GM08399 WT C/C | | GM09197 HD T₁₈₀ > C₁₇ | | GM04281 T₇₈/T₁₇ | |
| SGN002908_{C} | 0.67 | ND | 1.25 | ND | 1.1 | ND |
| SGN002911_{C} | 0.83 | ND | 1.13 | ND | 0.91 | ND |
| SGN004282_{T} | 1.22 | 0.87 | 0.69 | 0.62 | 0.66 | 0.43 |

ND = not determined

### Example 2. Off-Target Amplicon Sequencing

A bioinformatic off-target search was performed to identify potential off-target sites with homology of interest. As shown below in Table 19, off-targets were identified that contain a perfect PAM match that can be recognized by the RGN, and fewer than 5 mismatches that can include nucleotide substitutions or bulges. Amplicon sequencing was used to determine if there is off-target editing at bioinformatically identified sites (Table 21).

### Methods

### Bioinformatic Nomination

Potential off-target sites for the APG05586/SGN004282 composition were identified bioinformatically. Permissive blastn parameters were used to identify all possible regions of homology to the target sequence in the reference human genome (hg38). Once short regions of homology were identified, a sensitive Needleman-Wunsch secondary alignment with a custom scoring matrix was performed to identify the best alignment of the target to the off-target site. The custom scoring matrix is optimized to allow bulges in the DNA and RNA and correctly scores the nucleotides in the PAM region. Potential targets were selected for further evaluation if they had predicted off-targets with five or fewer total mismatches in the target sequence and a matching PAM sequence (NNRYA).

### Amplicon Sequencing

Total genomic DNA was harvested from Fibroblast cell lines edited with 2 μg APG05586-expressing mRNA and 4 μg SGN004282, iPSC derived Forebrain Neuron Progenitors edited with 2 μg APG05586 and 2 μg SGN004282, or unedited control cells. The total genomic DNA was then analyzed at all bioinformatically predicted off-target sites to determine if off-target editing has occurred. The off-target protocol design yields 84% power to repeatably detect small insertions and deletions with allele frequency greater than 0.1%. Deficiencies in read count marginally reduce the power. Estimated power is 84% at 100,000 reads (the target), 82% for 90,000 reads, 81% for 80,000 reads, and 79% for 70,000 reads. These estimates are based on repeatability of detection above the technology limited sensitivity, assuming an indel error rate of 0.00005 per read and ignoring indel length variation. Under these assumptions, a binomial model is used to estimate the repeatability by depth. Estimated power is based on the proportion of times we would accurately detect any indel under the stated conditions. The limit of detection was determined by the limit of the technology used, examination of historical negative controls, and high repeatability at and above this level of detection.

Oligonucleotides were produced to be used for Polymerase Chain Reaction (PCR) amplification and subsequent analysis of the amplified genomic target sites. Oligonucleotide sequences used are listed in Table 19. All oligonucleotides contain a Nextera adapter sequence added to the 5′ end as listed in Table 20.

The initial PCR reaction was performed in a 25 μl reaction as follows: 12.5 μl 2× Platinum SuperFi II PCR Master Mix, 1.25 μl pooled target specific forward and reverse primers, 1 μl genomic DNA, and 10.25 μl water. Thermocycling conditions were as follows: 98° C. for 1 min, [98° C. for 10 s, 60° C. for 10 s, 72° C. for 10 s]×35, 72° C. for 5 min, final hold at 4° C. The amplicons were visualized on an agarose gel, clean and concentrated and a second round of PCR was performed to add Illumina sequencing indices. The second amplification products were cleaned up and run on a TapeStation to quantify and check the purity of the samples to ensure only a single predominant product is present. The samples were then pooled, and deep sequencing was performed targeting 200,000 of250 b paired-end reads (2×100,000 reads) per amplicon.

The reads were analyzed using CRISPResso with custom parameters and an additional custom analysis was used to assess the specific substitution rate at the nucleotide of interest. Sequencing samples underwent a rigorous quality control analysis that includes minimum read count and alignment thresholds. Samples that had low alignment rates (less than 95% o of reads mapping to expected amplicon), high number of censored reads (more than 2500 of reads have multiple indels and substitutions indicating poor sequencing quality), or low read counts (less than 70,000 reads that map to the expected amplicon) failed quality control and were further evaluated by analyzing the reads aligned to the target sequence to determine pass/fail. Only sequencing samples that passed the quality control metrics were included in this analysis.

TABLE 19
Off-target sequences analyzed for the APG05586/SGN004282 composition.

| Off- | Off- | | | | | Left | Right |
|---|---|---|---|---|---|---|---|
| Target | Target | Accession | | Genome | | Primer | Primer |
| Number | Sequence | Number | Chromosome | Coordinates | PAM | Sequence | Sequence |
| 4282-1 | CCCAGC | NC_000011.10 | chr11 | 2899135- | GAGCA | TGATGC | TTCAGG |
| | CTCCAG | | | 2899171 | | AAGCTA | AAGCTG |
| | GATGCA | | | | | CCACCA | AGCCAC |
| | GTGTTC | | | | | GG (SEQ | TG (SEQ |
| | A (SEQ | | | | | ID NO: | ID NO: |
| | ID NO: | | | | | 41) | 42) |
| | 40) | | | | | | |
| 4282-2 | TCTGTC | NC_000009.12 | chr9 | 76309628- | CTGCA | CATGGC | TCACTC |
| | ATCCAG | | | 76309664 | | AAGTCC | TCACAC |
| | GTTGAA | | | | | CCGTCT | CCAAG |
| | GTGCAC | | | | | CT (SEQ | ATTCCA |
| | A (SEQ | | | | | ID NO: | (SEQ ID |
| | ID NO: | | | | | 44) | NO: 45) |
| | 43) | | | | | | |
| 4282-3 | CCCGTC | NC_000014.9 | chr14 | 60989675- | CAGCA | GTCCTA | GTCCCA |
| | CCCCAG | | | 60989711 | | GCTACT | GGGGT |
| | GCTGAA | | | | | GGGGC | ACACCA |
| | GTGCAA | | | | | AGC | ATGT |
| | (SEQ ID | | | | | (SEQ ID | (SEQ ID |
| | NO: 46) | | | | | NO: 47) | NO: 48) |
| 4282-4 | TCTGTC | NC_000022.11 | chr22 | 44422357- | TGGCA | GGACA | ATGTAG |
| | CTCCAG | | | 44422393 | | GGAGCT | CTGGGC |
| | GCTGAA | | | | | GGTCGT | ATGGTG |
| | GTGCAA | | | | | TGA | GT (SEQ |
| | (SEQ ID | | | | | (SEQ ID | ID NO: |
| | NO: 49) | | | | | NO: 50) | 51) |
| 4282-5 | TCTGTC | NC_000006.12 | chr6 | 106894656- | TGGTA | TTGCTT | AGCTAG |
| | CTCCAG | | | 106894692 | | ACCTTC | GCGTGG |
| | GCTGAA | | | | | CCCACT | TGGC |
| | GTGCAA | | | | | GG (SEQ | (SEQ ID |
| | (SEQ ID | | | | | ID NO: | NO: 54) |
| | NO: 52) | | | | | 53) | |
| 4282-6 | TCCGTC | NC_000007.14 | chr7 | 105725932- | TGGCA | CCACGC | ATGGA |
| | GTCCAG | | | 105725968 | | ATGGCC | AATTAG |
| | GATGG | | | | | TCCATT | GGCCG |
| | AGTGCA | | | | | TC (SEQ | GGCG |
| | A (SEQ | | | | | ID NO: | (SEQ ID |
| | ID NO: | | | | | 56) | NO: 57) |
| | 55) | | | | | | |
| 4282-7 | ACAGG | NC_000018.10 | chr18 | 8484887- | AGACA | CCGAA | CTTCTA |
| | CCCCCG | | | 8484923 | | ATCAGG | TGGTGG |
| | GTATGA | | | | | AAGGC | CACCGG |
| | AGTGCA | | | | | CCTT | TT (SEQ |
| | GCA | | | | | (SEQ ID | ID NO: |
| | (SEQ ID | | | | | NO: 59) | 60) |
| | NO: 58) | | | | | | |
| 4282-8 | TCTGTC | NC_000001.11 | chr1 | 119663552- | TGGCA | CGTGCT | AGTCCC |
| | CTCCAG | | | 119663588 | | CTGTTC | AGCTAC |
| | GATGA | | | | | CATCCC | TCAGGA |
| | AGTGCA | | | | | TT (SEQ | GG (SEQ |
| | A (SEQ | | | | | ID NO: | ID NO: |
| | ID NO: | | | | | 62) | 63) |
| | 61) | | | | | | |
| 4282-9 | ACTCTG | NC_000019.10 | chr19 | 45032187- | TGGCA | TAGCTG | CACTGT |
| | CCTCCA | | | 45032223 | | GGCGTG | GCTCCC |
| | GGCTGA | | | | | GTTGCA | AACTTG |
| | AGTGCA | | | | | (SEQ ID | CC (SEQ |
| | G (SEQ | | | | | NO: 65) | ID NO: |
| | ID NO: | | | | | | 66) |
| | 64) | | | | | | |

TABLE 20
Nextera adapter sequences

| Nextera | |
|---|---|
| Adapter | Nextera Adapter Sequence (5′→3′) |
| Left | TCGTCGGCAGCGTCAGATGTGTATAAGAGACAG |
| Primer | (SEQ ID NO: 137) |
| Sequence | |
| Right | GTCTCGTGGGCTCGGAGATGTGTATAAGAGACAG |
| Primer | (SEQ ID NO: 138) |
| Sequence | |

TABLE 21
Amplicon sequencing results

| Off-Target | | | Control | Reads in | Reads | Percent |
|---|---|---|---|---|---|---|
| Number | Sample ID | Cell Line | Type | Input | Aligned | Indels |
| 4282-1 | INDEL26898 | GM09197 | | 148015 | 147437 | 0 |
| | INDEL26899 | GM09197 | Negative | 129711 | 129283 | 0 |
| | INDEL26900 | GM04281 | | 143681 | 142906 | 0 |
| | INDEL26901 | GM04281 | Negative | 146826 | 146502 | 0 |
| | INDEL26902 | GM09197 | | 140103 | 139594 | 0 |
| | INDEL26903 | GM09197 | Negative | 140109 | 139815 | 0 |
| | INDEL26904 | GM04869 | | 147845 | 147464 | 0 |
| | INDEL26905 | GM04869 | Negative | 119861 | 119502 | 0 |
| | INDEL26906 | GM09197 | | 145106 | 144524 | 0 |
| | INDEL26907 | GM09197 | Negative | 189590 | 189139 | 0 |
| | INDEL26908 | GM04281 | | 128387 | 127706 | 0 |
| | INDEL26909 | GM04281 | Negative | 134186 | 133535 | 0 |
| | INDEL26981 | iPSC | | 210245 | 209195 | 0 |
| 4282-2 | INDEL26886 | GM09197 | | 196428 | 187816 | 0 |
| | INDEL26887 | GM09197 | Negative | 142998 | 137094 | 0 |
| | INDEL26888 | GM04281 | | 198226 | 190150 | 0 |
| | INDEL26889 | GM04281 | Negative | 185094 | 178763 | 0 |
| | INDEL26890 | GM09197 | | 193427 | 186952 | 0 |
| | INDEL26891 | GM09197 | Negative | 129229 | 124580 | 0 |
| | INDEL26892 | GM04869 | | 190550 | 183428 | 0 |
| | INDEL26893 | GM04869 | Negative | 174384 | 167649 | 0 |
| | INDEL26894 | GM09197 | | 179302 | 172839 | 0 |
| | INDEL26895 | GM09197 | Negative | 206270 | 198140 | 0 |
| | INDEL26896 | GM04281 | | 184895 | 177454 | 0 |
| | INDEL26897 | GM04281 | Negative | 172208 | 165805 | 0 |
| | INDEL26982 | iPSC | | 508352 | 491043 | 0 |
| | INDEL26990 | iPSC | | 115854 | 112249 | 0 |
| 4282-3 | INDEL26874 | GM09197 | | 122917 | 122714 | 0 |
| | INDEL26875 | GM09197 | Negative | 115522 | 115343 | 0 |
| | INDEL26876 | GM04281 | | 173714 | 173464 | 0 |
| | INDEL26877 | GM04281 | Negative | 159303 | 159098 | 0 |
| | INDEL26878 | GM09197 | | 181585 | 181290 | 0 |
| | INDEL26879 | GM09197 | Negative | 106935 | 106785 | 0 |
| | INDEL26880 | GM04869 | | 172677 | 172393 | 0 |
| | INDEL26881 | GM04869 | Negative | 78406 | 78260 | 0 |
| | INDEL26882 | GM09197 | | 126997 | 126809 | 0 |
| | INDEL26883 | GM09197 | Negative | 172340 | 172088 | 0 |
| | INDEL26884 | GM04281 | | 187392 | 187098 | 0.062887285 |
| | INDEL26885 | GM04281 | Negative | 141204 | 140977 | 0 |
| | INDEL26983 | iPSC | | 294532 | 293382 | 0.062800415 |
| 4282-4 | INDEL26862 | GM09197 | | 226144 | 218649 | 0 |
| | INDEL26863 | GM09197 | Negative | 171061 | 168447 | 0 |
| | INDEL26864 | GM04281 | | 210927 | 207057 | 0 |
| | INDEL26865 | GM04281 | Negative | 211018 | 208518 | 0 |
| | INDEL26866 | GM09197 | | 247511 | 243943 | 0 |
| | INDEL26867 | GM09197 | Negative | 174800 | 173463 | 0 |
| | INDEL26868 | GM04869 | | 250837 | 248577 | 0 |
| | INDEL26869 | GM04869 | Negative | 201316 | 198276 | 0.084624436 |
| | INDEL26870 | GM09197 | | 194329 | 191560 | 0 |
| | INDEL26871 | GM09197 | Negative | 185000 | 181411 | 0 |
| | INDEL26872 | GM04281 | | 179243 | 173937 | 0 |
| | INDEL26873 | GM04281 | Negative | 211849 | 199197 | 0 |
| | INDEL26984 | iPSC | | 330830 | 323722 | 0.044591100 |
| | INDEL26992 | iPSC | Negative | 209118 | 204127 | 0 |
| 4282-5 | INDEL31695 | GM09197 | | 169310 | 164162 | 0 |
| | INDEL31696 | GM09197 | Negative | 165817 | 163748 | 0 |
| | INDEL31697 | GM04281 | | 185853 | 181183 | 0 |
| | INDEL31698 | GM04281 | Negative | 181855 | 179176 | 0 |
| | INDEL31699 | GM09197 | | 183950 | 182152 | 0 |
| | INDEL31700 | GM09197 | Negative | 206282 | 203963 | 0 |
| | INDEL31701 | GM04869 | | 168507 | 166239 | 0 |
| | INDEL31702 | GM04869 | Negative | 132938 | 130486 | 0 |
| | INDEL31711 | GM09197 | | 192244 | 190281 | 0.100678112 |
| | INDEL31712 | GM09197 | Negative | 149942 | 148652 | 0 |
| | INDEL31713 | GM04281 | | 152374 | 150511 | 0 |
| | INDEL31714 | GM04281 | Negative | 200780 | 197279 | 0 |
| | INDEL31719 | iPSC | | 166527 | 161089 | 0 |
| | INDEL31720 | iPSC | Negative | 175137 | 171678 | 0 |
| 4282-6 | INDEL37117 | GM09197 | | 347581 | 346951 | 0.217316 |
| | INDEL37118 | GM09197 | Negative | 285079 | 283939 | 0 |
| | INDEL37119 | GM04281 | | 371337 | 370362 | 0.148185 |
| | INDEL37120 | GM04281 | Negative | 315784 | 315136 | 0.565103 |
| | INDEL37121 | GM09197 | | 311749 | 310908 | 0.107854 |
| | INDEL37122 | GM09197 | Negative | 300631 | 299271 | 0.74272 |
| | INDEL37123 | GM04869 | | 322399 | 321599 | 0.579547 |
| | INDEL37124 | GM04869 | Negative | 321849 | 321322 | 0 |
| | INDEL37125 | GM09197 | | 339365 | 338219 | 0.967144 |
| | INDEL37126 | GM09197 | Negative | 330372 | 329674 | 0.042466 |
| | INDEL37127 | GM04281 | | 284955 | 280987 | 1.5269 |
| | INDEL37128 | GM04281 | Negative | 256911 | 254782 | 0.97614 |
| | INDEL39149 | iPSC | | | | |
| | INDEL31722 | iPSC | Negative | 106581 | 106184 | 0.229446 |
| 4282-7 | INDEL26826 | GM09197 | | 214424 | 214121 | 0.058858152 |
| | INDEL26827 | GM09197 | Negative | 152936 | 152698 | 0 |
| | INDEL26828 | GM04281 | | 220077 | 219766 | 0.047168503 |
| | INDEL26829 | GM04281 | Negative | 198914 | 198656 | 0 |
| | INDEL26830 | GM09197 | | 266657 | 266311 | 0.097557643 |
| | INDEL26831 | GM09197 | Negative | 152803 | 152592 | 0 |
| | INDEL26832 | GM04869 | | 232183 | 231860 | 0.100801613 |
| | INDEL26833 | GM04869 | Negative | 165649 | 165406 | 0.099152309 |
| | INDEL26834 | GM09197 | | 249982 | 249652 | 0.049107143 |
| | INDEL26835 | GM09197 | Negative | 262198 | 261860 | 0.094412995 |
| | INDEL26836 | GM04281 | | 189205 | 188887 | 0 |
| | INDEL26837 | GM04281 | Negative | 192608 | 192310 | 0 |
| | INDEL26985 | iPSC | | 274463 | 273426 | 0.093491230 |
| | INDEL26993 | iPSC | Negative | 412860 | 411445 | 0.093816946 |

| | |
|---|---|
| 4282-8 | Primer sets evaluated failed to provide satisfactory sequencing |
| 4282-9 | Primer sets evaluated failed to provide satisfactory sequencing |

### Results

No bona fide off-target sites were identified with APG05586-SGN004282 with a limit of detection of 0.1%.

Two off-target sites, 4282-8 and 4282-9, remain difficult to amplify and sequence.

Off target 4282-8 is in an intronic region of LOC124904390. This non-coding RNA has been shown to be expressed in the brain but has no known function. This region (+−500 bp from the offtarget start site) is known to contain deletions and substitutions. It has a total of four mismatches, with no mismatches in the seed region. Off target editing at this location is not likely to cause cellular changes.

Off target 4282-9 is in an intronic region of RELB proto-oncogene, NF-kB subunit (RELB). It has broad expression in many tissues, with highest expression in bone marrow and appendix. This region (+/−500 bp from the off-target start site) is known to contain deletions and substitutions. It has a total of five mismatches, with one mismatch in the seed region. As this is an intronic region, off-target editing at this location is not likely to cause cellular changes. However, as this falls within a proto-oncogene, it might be beneficial to try another method to assess off target at this site (rhamp-seq or target capture).

### Example 3. Editing in iPSC-Derived Forebrain Neurons and Medium Spiny Neurons

The objective of this study was to determine if APG07433.1, along with SGN002908 (C guide), SGN002911 (C guide), and/or APG05586, along with SGN004281 (T guide), and SGN004282 (T guide), is able to edit HTT in iPSC-derived forebrain neurons and medium spiny neurons.

### Methods

iPSC-Derived Forebrain Neuron Differentiation

iPSCs were differentiated to forebrain neuron using manufacturer's manual for STEMdiff™ SMADi Neural Induction kit, followed by STEMDiff™ Forebrain Neuron Differentiation Kit and STEMDiff™ Forebrain Neuron Maturation Kit (StemCell Technologies).

### Medium Spiny Neuron Differentiation

iPSCs were differentiated to medium spiny neurons using a protocol modified slightly from a previously published method. (Grigor'eva et al. Cytotechnology 72, 649-663 (2020); DOI: 10.1007/s10616-020-00406-7).

Characterization of iPSC Derived Forebrain Neurons

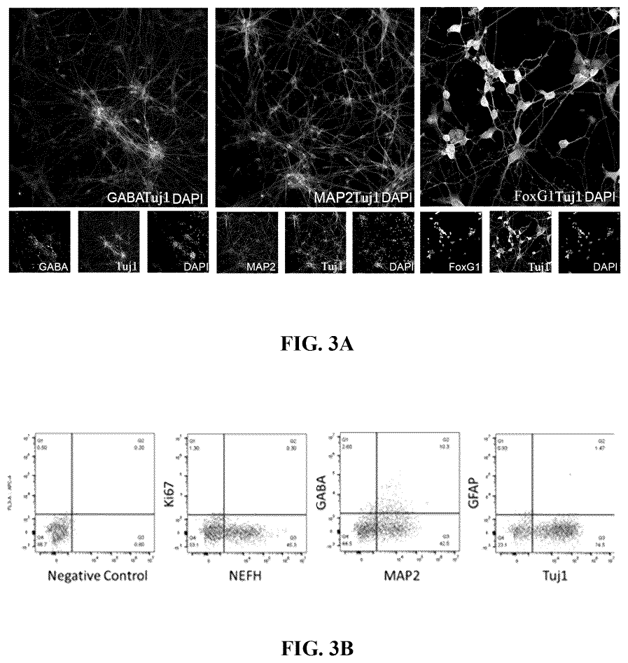

To confirm neuron differentiation, iPSC-derived forebrain neurons were cultured on chamber slides or 24 well plates for immunofluorescent analysis of neuronal marker genes. Briefly, cells were fixed by 4% Paraformaldehyde and permeabilized using 0.1% Triton x-100 at RT for 10 mins. The cells were blocked with 5% normal goat serum and stained with primary antibodies anti-GABA, anti-MAP2 or anti-β-III-tubulin (Tuj1) overnight at 4° C. Anti-Mouse IgG Alexa 488, and Alexa 555, and anti-Rabbit IgG Alexa 488 and Alexa 555 were used for secondary staining and photographed by confocal microscope. See results in FIG. 3A which demonstrate the iPSC-derived neuron type cells contain a highly pure population of Tuj1 (green) with partly positive for GABA and MAP2 (red) and highly pure positive for FoxG1 (red), indicating a forebrain-type identity. Antibody based staining demonstrates that iPSC derived cells have been successfully differentiated into mature, GABA-expressing neurons.

FACS analysis was performed on the iPSC-derived forebrain neurons. Briefly, a single cell suspension was obtained by disassociating iPSC-derived forebrain neurons. The resulting single cell suspension was then used for staining with primary antibodies using the manufacturer's intracellular staining protocol. 2E+5 cells were stained with the following primary antibodies: Ki67, NEFH, MAP2, GABA, GFAP, Tuj1. Secondary antibodies, including Anti-Mouse IgG Alexa Fluor™ 488 and Anti-Rabbit IgG Alexa Fluor™ 647, were also used. For FACS analysis, Beckman Cytoflex was used. The results from FACS analysis are shown in FIG. 3B. Ki67 is a marker of cell proliferation, whereas NEFH (neurofilimants) is an intermediated filament protein found specifically in neurons, and has a crucial role in mature axons. GABA is a neurotransmitter in the central nervous system, and MAP2 (microtubule associated protein 2) is involved in microtubule assembly, an essential step in neurogenesis. Tuji1 is a beta III-tubulin isoform that is predominantly present in cells of neuronal origin, and is one of the earliest markers of neuronal differentiation. GFAP is heavily and specifically expressed in astrocytes and certain astroglia of the central nervous system. The FACS results show that the iPSC-derived forebrain neurons are negative for Ki67, indicating that the cells are in a postmitotic state. Only 2.4% of the cells are positive for GFAP, which is a specific marker for astrocytes. 74.5% of the cells are Tuj1+/GFP−, and 45.3% are positive for NEFH.

Characterization of iPSC Derived Medium Spiny Neurons (MSNs)

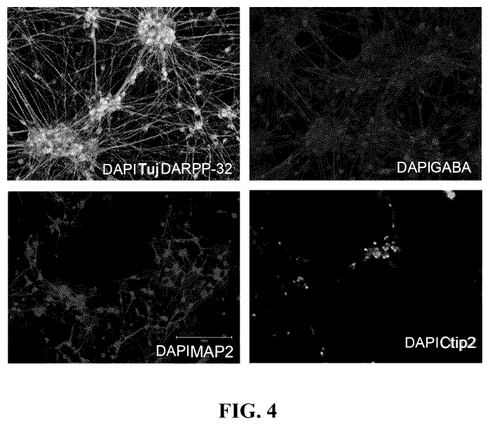

To confirm MSN differentiation, iPSC-derived medium spiny neurons were cultured on 24 well plates for immunofluorescent analysis of neuronal marker genes. Briefly, cells were fixed by 4% paraformaldehyde and permeabilized using 0.1% Triton x-100 at RT for 10 mins. The cells were blocked with 5% normal goat serum and stained with primary antibodies, Anti-DARPP32, anti-GABA, anti-MAP2 or anti-β-III-tubulin and Anti-Ctip2 overnight at 4° C. Anti-Mouse IgG Alexa 488, and Alexa 555, and anti-Rabbit IgG Alexa 488 and Alexa 555 were used for secondary staining. Photographed by EVOS 5000 microscope. See results in FIG. 4. Antibody based staining demonstrates that iPSC derived cells have been successfully differentiated into medium spiny neurons.

### Nucleofection Conditions

A combination of 2 μg APG05586 mRNA and 2 μg of either SGN004281 or SGN004282 was employed to edit the iPSC-derived cells. Alternatively, a combination of 2 μg of APG07433.1 mRNA and 2 μg of either SGN002908 or SGN002911 was employed. For nucleofection of iPSCs, program CB-150 and P3 buffer were used, while program EM-110 and p3 buffer were used for other iPSC-derived cells.

### Sample Collection

Cell samples of induced pluripotent stem cells (iPSCs) and neural progenitor cells (NPCs) were collected at day 4 post nucleofection. Cell samples of forebrain neuron progenitors (FBPs) and forebrain neurons (FBNs) were collected at day 6 post nucleofection.

### Analysis of Indel Formation

Genomic DNA was isolated from 5e+5 cells via an isolation kit according to the manufacturer's instructions. Concentrations were determined and 20 ng-40 ng of gDNA was used for subsequent assays. Polymerase chain reaction (PCR) was run using HTT-4 primers at 2 μM with Platinum SuperFI II taq polymerase (ThermoFisher) according to the manufacturer's instructions.

The cycling conditions are shown in Table 22.

TABLE 22
Cycling conditions for HTT PCR.

| | | | |
|---|---|---|---|
| Cycle step | Temperature | Time | Cycles |

| | | | | | |
|---|---|---|---|---|---|
| Initial denaturation | 98° | C. | 30 | seconds | 1 |
| Denaturation | 98° | C. | 10 | seconds | 35 |
| Annealing | 60° | C. | 10 | seconds | |
| Extension | 72° | C. | 15 | seconds | |
| Final extension | 72° | C. | 5 | minutes | 1 |

| | | | |
|---|---|---|---|
| End | 4° | C. | Hold |

Amplicons were visualized via flash gel run at 225V for 4 mins. PCR products were then subsequently purified and concentrated and sequenced.

### Results

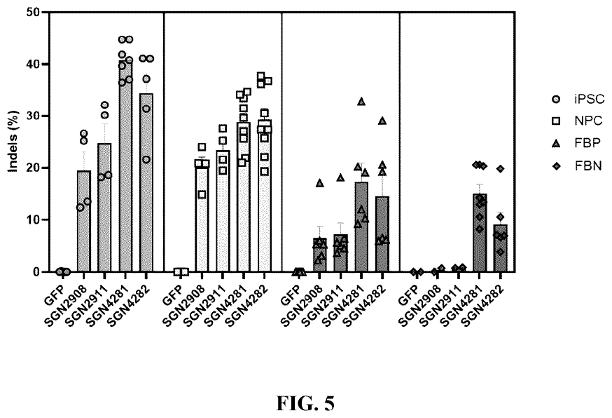

The percent of indels is provided in Table 23 and FIG. 5.

TABLE 23
Percent of indels in nucleofected induced pluripotent stem cells (iPSCs), neural progenitor
cells (NPCs), forebrain neuron progenitors (FBPs), and forebrain neurons (FBNs)

| GFP | SGN2908 | SGN2911 | SGN4281 | SGN4282 | GFP | SGN2908 | SGN2911 | SGN4281 | SGN4282 |
|---|---|---|---|---|---|---|---|---|---|
| iPSCs | | | | | NPCs | | | | |
| 0.15 | 26.6 | 30.12 | 40.78 | 41.09 | 0 | 20.81 | 21.54 | 33.46 | 36.14 |
| 0 | | | 41.87 | 37.18 | 0 | | | 31.5 | 36.74 |
| 0 | 25.2 | 32.13 | 44.83 | 41.05 | 0 | 24 | 27.54 | 29.59 | 30.5 |
| 0 | 13.5 | 18.59 | 37.05 | 31.4 | | | | 21.87 | 19.24 |
| 0 | 12.35 | 18.15 | 36.47 | 21.57 | 0 | | | 20.96 | 22.06 |
| | | | 39.67 | | 0 | 20.76 | 25.22 | 25.65 | 27.36 |
| | | | 44.8 | | | 14.8 | 19.41 | 34.13 | 27.3 |
| | | | | | | | | 27 | 25.7 |
| FBPs | | | | | FBNs | | | | |
| 0 | 17.06 | 18.1 | 32.85 | 29.04 | 0 | 0.69 | 0.75 | 20.55 | 6.6 |
| 0 | 5.3 | 5 | 9.2 | 6.5 | 0 | 0 | 0.85 | 20.52 | 6.91 |
| 0 | 5.96 | 3.6 | 11.96 | 5.89 | 0 | | | 14.24 | 10.5 |
| 0 | 5.26 | 4.48 | 10.27 | 6.16 | 0 | | | 13.37 | 3.82 |
| 0 | 3.08 | 6.39 | 19.07 | 19.21 | | | | 20.3 | 7.08 |
| 0 | 2.2 | 5.61 | 20.2 | 20.55 | | | | 8.2 | 19.8 |
| | | | | | | | | 12.9 | |

The results from this study demonstrate the efficacy of APG07433.1 and APG05586 nucleases in generating indels in different types of cells derived from induced pluripotent stem cells (iPSCs). These findings reveal that the use of APG07433.1 nuclease with SGN002908 or SGN002911 resulted in 20% o and 25% indels in iPSCs and NPCs, while forebrain neuron progenitors exhibited 8.4% o and 7.8% o indels, respectively. Interestingly, no indels were observed in forebrain neurons. In contrast, the use of APG05586 nuclease with SGN004281 or SGN004282 resulted in a higher frequency of indels, with 41% and 35% in iPSCs, 29% in NPCs, and 16% and 12% in forebrain neuron progenitors. Forebrain neurons showed a lower frequency of indels with 16% and 9% for the same nuclease systems.

Furthermore, this study also assessed the impact of combining the nucleases with GFP mRNA or GFP alone, and found that no indels were observed in any of the nucleofected cells. These results indicate that the combination of APG07433.1 or APG05586 nucleases with guide RNA systems is a better gene editing approach, with the latter showing higher efficacy in inducing indels, particularly in iPSC derived cells, including forebrain neurons.

In conclusion, these studies highlight the importance of choosing appropriate nucleases and guide RNA systems for efficient and specific gene editing in iPSCs and their derivatives. The results of these studies demonstrate that the APG05586 nuclease, in combination with guide RNA, exhibits a remarkable efficiency in gene editing for Huntington's disease.

### Example 4. AAV Delivery to YAC128 Mouse

The previous in vitro studies described above used AAV2 or AAV6 which efficiently transduce in vitro cells, but for neurons in vivo, AAV5-delivered constructs are preferred. These studies evaluated the in vivo dose response and biodistribution of AAV5-delivered APG07433.1 nuclease and single guide RNA (SGN) following intrastriatal stereotaxic delivery of pAAV-hU6(318 bp)-SGN002908-JeT-APG07433.1-SV40pA (SEQ ID NO: 30) packaged in AAV5. The RGN-SGN is specifically designed to delete rs362331 SNP in exon 50 of the specific mutant allele to suppress function of the human mutant huntingtin (mutHTT) gene which encodes the human mutHTT protein. Reduced mutHTT protein levels have been shown preclinically to halt or slow disease progression.

### Methods

### Animals

Treatment of the animals was in adherence to the regulations outlined in the USDA Animal Welfare Act (9 CFR, Parts 1, 2, and 3) and the conditions specified in The Guide for Care and Use of Laboratory Animals (ILAR publication, 1996, National Academy Press). The protocol was approved by the vivarium's Institutional Animal Care and Use Committee (IACUC) prior to initiation of the study.

The FVB-Tg(YAC128)53Hay/J (YAC128) transgenic mouse expresses multiple copies of full-length human mutant HTT (humutHTT) with 100 and 126 glutamine repeats, endogenous mouse HTT (musHTT), and contains the target SNP allele of the proposed therapeutic test article (Slow et al., 2003, Human Molecular Genetics 12(13):1555-1567; Southwell et al., 2013, Human Molecular Genetics 22(1):18-34). This model has been extensively studied and recapitulates HD associated neuropathological phenotypes (Slow et al., 2003; Raamsdonk et al., 2007, Neurobiology of Disease 26:189-200).

The animals used in this study were obtained from Jackson Laboratories, Bar Harbor, ME. The animals were purpose-bred and were experimentally naïve at the outset of the study.

### Surgical Procedure

For each surgery, the animal was administered Meloxicam (2 mg/kg, Lot no: 1375-90B Covetrus, Portland, ME, USA) 30 min prior to surgery and every 24 hours for 72 hours post-surgery via subcutaneous injection. Prior to initiation of surgery, the animal is anesthetized with 5% isoflurane in an induction chamber and then transferred to the NeuroStar stereotactic frame (Robot Stereotaxic Serial #SD733, NeuroStar, Tubingen, Germany). Upon placement in the stereotactic frame, the head of the animal is secured in place by ear bars and bite-bar. Anesthesia is maintained throughout the surgery with 2% isoflurane. The animal is placed in prone position on a heating pad at 37° C. throughout the surgical procedure. Topical ophthalmic ointment is applied to both eyes to prevent ocular dryness/injury during the surgery. The scalp is disinfected with a povidone-iodine solution, followed by 70% ethanol, before initial incision is made in scalp to reveal the skull landmarks bregma and lambda. The exposed skull is disinfected with 70% alcohol. The relative position of the injection sites to the robot are determined by the built-in software of the Neurostar robot based on the relative locations of the bregma and lambda. Head tilt is calibrated based on the relative skull position 2 mm to the left and 2 mm to the right of the sagittal suture. For each surgery, the animal is injected at 4 sites: Sites 1/3: Anterior-Posterior (AP)+1.0 mm, Medial-Lateral (ML)±1.6 mm, Dorsal-Ventral (DV)+3.8 mm; and sites 2/4: AP+0.0 mm, ML±2.3 mm, DV+3.7 mm, relative to the Bregma. All four coordinate positions are defined by the stereotactic software and used to guide drilling and injection position. For each injection site, a burr hole is made in the skull with an automated drill with specialized drill bit (Fine Science Tools, Burrs for Micro Drill, 1.4 mm, catalogue no. NC9727231) guided by the software-defined AP-ML coordinates. The injection is carried out using a 10 μl Hamilton syringe (Neuros Syringe, Model 1701, RN, 33 gauge, Point Style 3). The syringe needle is inserted and lowered to the defined coordinates through the burr hole. Total administration volumes were 2 μl/site delivered at 0.2 μl/min injection rate (yielding a total 4 μl/hemisphere and 8 μl/brain). The needle is left in place at the injection site for 5 minutes following completion of infusion and then gradually retracted. The scalp is sealed by tissue adhesive after all 4 sites are injected. The animal is placed in a clean cage on top of a heating pad to maintain physiological body temperature while recovering from anesthesia. The animal is then placed back in its home cage for in-life observation.

All animals were checked for general health, morbidity, and mortality twice daily performed by vivarium husbandry staff. All animals were weighed pre-treatment, 2-3 times weekly after intrastriatal (IS) injection and prior to termination. In addition, clinical observations were performed daily the first three days after injection and then 2-3 times weekly afterwards coinciding with bodyweight measurements.

Euthanasia occurred via isoflurane overdose after blood sample collection. Animals were transcardially perfused with 10 ml PBS. The whole brains were dissected. The cortex, striatum, and cerebellum were micro-dissected. Tissues were either flash frozen on dry ice or submerged in 10× volume of tissue mass of RNAlater immediately after dissection. Samples were kept at <−60° C. prior to being evaluated.

### Administration of AAV

pAAV-hU6(318 bp)-SGN002908-JeT-APG07433.1-SV40pA (SEQ ID NO: 30) packaged in AAV5 was delivered to the striatum of YAC128 mice in three escalating doses of 1.3E10, 6.4E10, and 3.6E11 vg, and differing volumes (4-8 μL), with resultant biodistribution and pharmacodynamic assessment at 4 weeks and 3 months. Data are compared with concurrent vehicle-treated and naïve controls.

Each animal in treatment groups received bilateral stereotaxic injection in the striatum. Animals were injected at 4 sites with 1-2 μl/site at 0.2 μl/min (total 2-4 μl/hemisphere). A stereotaxic robot was used to perform the injection. Injection coordinates for the four separate sites were: Sites 1/3—Anterior-Posterior (AP)+1.0 mm, Medial-Lateral (ML)±1.6 mm, Dorsal-Ventral (DV) 3.8 mm; and sites 2/4—AP+0.0 mm, ML±2.3 mm, DV 3.7 mm, relative to the Bregma.

### Sample Preparation

At 4 weeks or 3 months post administration, euthanasia occurred via isoflurane overdose. Animals were transcardially perfused with 10 ml PBS. The whole brain was dissected and brain tissues micro-dissected. Tissues were either flash frozen on dry ice or submerged in 10× volume of tissue mass of RNAlater immediately after dissection. Samples were kept in a −80° C. freezer prior to being evaluated.

RNA, DNA, or protein lysate was extracted from tissues of interest. The expression of human mutHTT and RGN nuclease levels were evaluated through qPCR with mRNA or Jess capillary western blot with the protein lysate. AAV5 genome distribution was evaluated with dPCR. Genome editing efficacy was evaluated with NGS.

Data was expressed as mean±SE. Comparisons of parameters was performed using a one-way analysis of variance followed by a post hoc Dunnet's test. Statistical significance is taken as P<0.05.

### Sample Analysis—AAV Vector Genomes (Biodistribution)

Genomic DNA was isolated using the Maxwell RSC 48 instrument (Promega, Madison, WI). Absolute quantification of vector genome copy number was performed based on amplicons targeting APG07433.1 using the following primers and probe: F 5′-CATTACAGGCGCTGCTCATA-3′ (SEQ ID NO: 67), R 5′-TTGCCCTTCTTGTCAATTCC-3′ (SEQ ID NO: 68), probe: TCCCTTCCGCATGATGGTCTGCT (SEQ ID NO: 69). Mouse eukaryotic translation elongation factor 2 (Eef2) expression was used as loading reference.

Sample Analysis—APG07433.1 mRNA

mRNA was isolated and reverse transcription was carried out. Quantification of APG07433.1 expression was elucidated based on amplicons targeting APG07433.1 using the following primers and probe: F 5′-CATTACAGGCGCTGCTCATA-3′ (SEQ ID NO: 72), R 5′-TTGCCCTTCTTGTCAATTCC-3′ (SEQ ID NO: 73), probe: TCCCTTCCGCATGATGGTCTGCT (SEQ ID NO: 74). Mouse eukaryotic translation elongation factor 2 (Eef2) expression was used as loading reference.

Sample Analysis—Human mutHTT INDELs

Genomic DNA was isolated and the mutHTT INDEL rate was evaluated based on amplicon sequencing of targeted region with the following PCR with primers: Left 5′-TCGTCGGCAGCGTCAGATGTGTATAAGAGACAGAAACGAAGGTACACGAGTGG-3′ (SEQ ID NO: 70), and Right 5′-GTCTCGTGGGCTCGGAGATGTGTATAAGAGACAGGCAAACAGGCAGCACAAAAT-3′ (SEQ ID NO: 71). Purified amplicons were then sequenced.

### Sample Analysis—HTT Protein

Protein lysate was prepared with sonication and sample buffer. HTT protein levels were detected using Jess capillary western blot with mouse anti-HTT antibody. Mouse anti-vinculin antibody was used as loading control.

### Results

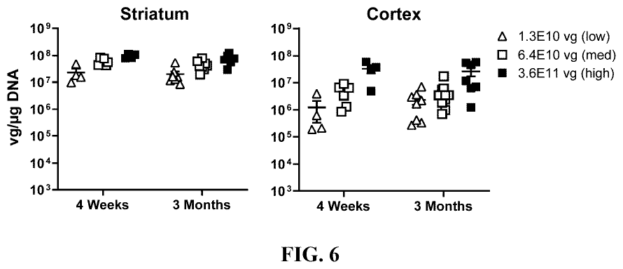

Intrastriatal injections of AAV5-JeT-APG07433.1-SGN002908 were well tolerated. Dose-dependent transduction of the striatum was evident at both 4 weeks and 3-months post administration (FIG. 6). AAV5 vector disposition was dose proportional with 2.27×10⁷, 5.88×10⁷, and 9.51×10⁷ vg/μg evident at 1.3E10, 6.4E10 and 3.6E11 vg, respectively. Vector levels in the striatum were comparable at both assessed time points of 4 weeks and 3 months. Substantial levels of vector DNA were also present in the cortex indicating retrograde transport from the administration site within the striatum. Dose-dependent AAV5 cortex levels of 1.24×10⁶, 4.65×10⁶, 3.29×10⁷ vg/μg were observed at 1.3E10, 6.4E10 and 3.6E11 vg, respectively corresponding to 94.5, 92.1, and 65.4 percent lower levels compared to striatal levels.

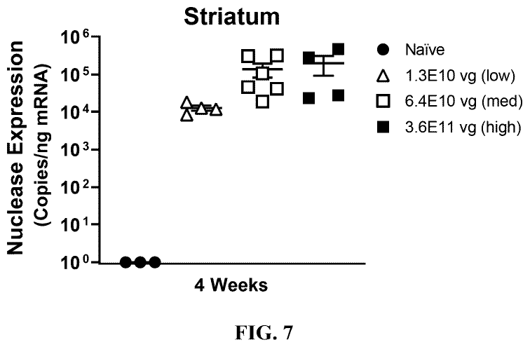

A dose-dependent increase in APG07433.1 transgene expression was observed with 1.27×10⁴, 1.39×10⁵, and 1.99×10⁵ copies/ng RNA present within the striatum at 1.3E10, 6.4E10 and 3.6E11 vg, respectively (FIG. 7).

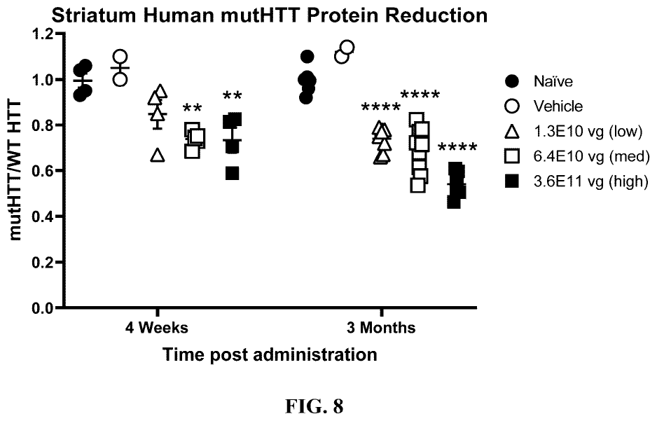

Administration of AAV5-JeT-APG07433.1-SGN002908 led to 15.3, 26.1 and 26.5% reductions in human mutHTT following administration of 1.3E10, 6.4E10 and 3.6E11 vg, respectively when evaluated at 4-weeks with reductions 26.0, 30.5 and 45.9% at 3 months (FIG. 8).

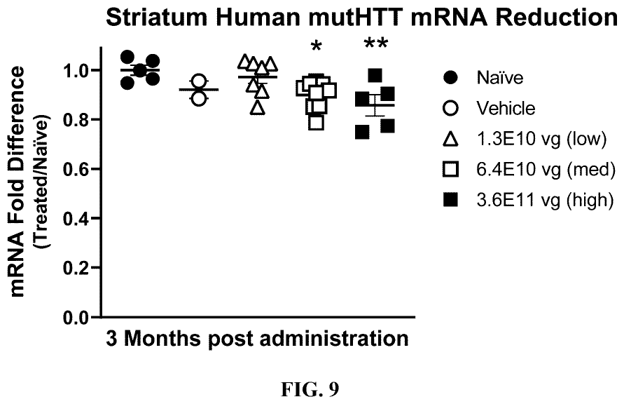

Dose dependent mutant HTT mRNA reduction was observed at 3-months following AAV5-JeT-APG07433.1-SGN002908 administration. Intrastriatal administration of 1.3E10, 6.4E10 and 3.6E11 vg led to reductions of 2.7, 10.1 and 14.2% respectively, compared to naïve animals (FIG. 9).

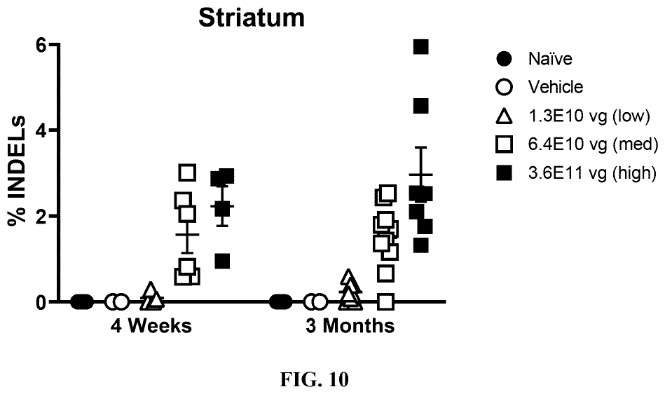

Editing was evaluated using Next Generation Sequencing (NGS): Genomic DNA was analyzed with NGS using amplicon sequencing directed to the site of interest. Intrastriatal administration of AAV5-JeT-APG07433.1-SGN002908 led to dose dependent increases in INDEL formation in the striatum when evaluated at both 4-weeks and 3-months. Administration of 1.3E10, 6.4E10 and 3.6E11 vg generated 0.09, 1.57, 2.27% INDELs at 4-weeks and 0.23, 1.50, and 2.96% at 3-months, respectively (FIG. 10).

### Example 5. AAV Delivery to BACHD Mouse

### Methods

### Animals

Treatment of the animals was in adherence to the regulations outlined in the USDA Animal Welfare Act (9 CFR, Parts 1, 2, and 3) and the conditions specified in The Guide for Care and Use of Laboratory Animals (ILAR publication, 1996, National Academy Press). The protocol was approved by the vivarium's Institutional Animal Care and Use Committee (IACUC) prior to initiation of the study.

The FVB/N-Tg(HTT*97Q)IXwy/J (BACHD) transgenic mouse expresses multiple copies of full-length human mutHTT with 97 glutamine repeats, endogenous mouse HTT, and contains the target SNP allele of the proposed therapeutic test article (Slow et al., 2003, Human Molecular Genetics 12(13):1555-1567; Southwell et al., 2013, Human Molecular Genetics 22(1):18-34). This model has been extensively studied and recapitulates HD associated neuropathological phenotypes (Gray et al., 2008, The Journal of Neuroscience 28(24):6182-6195, Pouladi et al., 2012, Hum Mol Genet 21(10):2219-2232). In preclinical studies, the BACHD murine HD model has been used to demonstrate in vivo selective reduction of mutHTT level with antisense oligonucleotides targeting SNPs (Carroll et al., 2011, Molecular Therapy 19(12):8-2185). The animals used in this study were obtained from Jackson Laboratories, Bar Harbor, ME. The animals were purpose-bred and were experimentally naïve at the outset of the study.

### Surgical Procedure

For each surgery, the animal was administered Meloxicam (2 mg/kg, Lot no: 1375-90B Covetrus, Portland, ME, USA) 30 min prior to surgery and every 24 hours for 72 hours post-surgery via subcutaneous injection. Prior to initiation of surgery, the animal is anesthetized with 5% isoflurane in an induction chamber and then transferred to the NeuroStar stereotactic frame (Robot Stereotaxic Serial #SD733, NeuroStar, Tubingen, Germany). Upon placement in the stereotactic frame, the head of the animal is secured in place by ear bars and bite-bar. Anesthesia is maintained throughout the surgery with 2% isoflurane. The animal is placed in prone position on a heating pad at 37° C. throughout the surgical procedure. Topical ophthalmic ointment is applied to both eyes to prevent ocular dryness/injury during the surgery. The scalp is disinfected with a povidone-iodine solution, followed by 70% ethanol, before initial incision is made in scalp to reveal the skull landmarks bregma and lambda. The exposed skull is disinfected with 70% alcohol. The relative position of the injection sites to the robot are determined by the built-in software of the Neurostar robot based on the relative locations of the bregma and lambda. Head tilt is calibrated based on the relative skull position 2 mm to the left and 2 mm to the right of the sagittal suture. For each surgery, the animal is injected at 4 sites: Sites 1/3: Anterior-Posterior (AP)+1.0 mm, Medial-Lateral (ML)±1.6 mm, Dorsal-Ventral (DV)+3.8 mm; and sites 2/4: AP+0.0 mm, ML±2.3 mm, DV+3.7 mm, relative to the Bregma. All four coordinate positions are defined by the stereotactic software and used to guide drilling and injection position. For each injection site, a burr hole is made in the skull with an automated drill with specialized drill bit (Fine Science Tools, Burrs for Micro Drill, 1.4 mm, NC9727231) guided by the software-defined AP-ML coordinates. The injection is carried out using a 10 μl Hamilton syringe (Neuros Syringe, Model 1701, RN, 33 gauge, Point Style 3). The syringe needle is inserted and lowered to the defined coordinates through the burr hole. Total administration volumes were 2 μl/site delivered at 0.2 μl/min injection rate (yielding a total 4 μl/hemisphere and 8 μl/brain). The needle is left in place at the injection site for 5 minutes following completion of infusion and then gradually retracted. The scalp is sealed by tissue adhesive after all 4 sites are injected. The animal is placed in a clean cage on top of a heating pad to maintain physiological body temperature while recovering from anesthesia. The animal is then placed back in its home cage for in-life observation.

All animals were checked for general health, morbidity, and mortality twice daily performed by vivarium husbandry staff. All animals were weighed pre-treatment, 2-3 times weekly after IS injection and prior to termination. In addition, clinical observations were performed daily for the first three days after injection and then 2-3 times weekly afterwards coinciding with bodyweight measurements.

Euthanasia occurred via isoflurane overdose after blood sample collection. Animals were transcardially perfused with 10 ml PBS. The whole brains were dissected. The cortex, striatum, and cerebellum were micro-dissected. Tissues were either flash frozen on dry ice or submerged in 10× volume of tissue mass of RNAlater immediately after dissection. Samples were kept in <−60° C. prior to being evaluated.

### Analysis of Indel Formation

Genomic DNA was isolated and absolute quantification of vector genome copy number was determined based on amplicons targeting APG07433.1 using the following primers and probe: F 5′-CATTACAGGCGCTGCTCATA-3′ (SEQ ID NO: 67), R 5′-TTGCCCTTCTTGTCAATTCC-3′ (SEQ ID NO: 68), probe: TCCCTTCCGCATGATGGTCTGCT (SEQ ID NO: 69). Mouse eukaryotic translation elongation factor 2 (Eef2) expression was used as loading reference. INDEL rate was evaluated based on amplicon sequencing of targeted region with following PCR with primers: Left 5′-TCGTCGGCAGCGTCAGATGTGTATAAGAGACAGAAACGAAGGTACACGAGTGG-3′ (SEQ ID NO: 70), and Right 5′-GTCTCGTGGGCTCGGAGATGTGTATAAGAGACAGGCAAACAGGCAGCACAAAAT-3′ (SEQ ID NO: 71). Purified amplicons were sequenced and mRNA was isolated. Reverse transcription was carried out with SuperScript™ IV reverse transcriptase (Cat #18090010, Invitrogen, Waltham, MA). Quantification of APG07433.1 expression was performed based on amplicons targeting APG07433.1 using the following primers and probe: F 5′-CATTACAGGCGCTGCTCATA-3′ (SEQ ID NO: 72), R 5′-TTGCCCTTCTTGTCAATTCC-3′ (SEQ ID NO: 73), probe: TCCCTTCCGCATGATGGTCTGCT (SEQ ID NO: 74). Mouse eukaryotic translation elongation factor 2 (Eef2) expression was used as loading reference.

Protein lysate was prepared with sonication and sample buffer. HTT protein levels were detected using Jess capillary western blot with mouse anti-HTT antibody. Mouse anti-vinculin antibody was used as loading control.

### Example 5.1 Evaluation of AAV5-hU6-SGN4282-JeT-APG05586 to Reduce Mutant Huntingtin Twelve Weeks Following Intrastriatal Administration in BACHD Mice

The objective of these studies was to evaluate the activity of intrastriatally AAV5-delivered APG05586 nuclease and single guide RNA to delete the rs362331 SNP in exon 50 of the specific mutant allele and suppress the mutant huntingtin (mutHTT) gene which encodes mutHTT protein. Reduced mutHTT levels have been shown preclinically to halt or slow disease progression. The activity of pAAV-hU6(318 bp)-SGN004282-Jet-APG005586-SV40pA (SEQ ID NO: 32) packaged in AAV5 was evaluated 4 weeks following intrastriatal administration of 1.72E11 vg in the clinically relevant BACHD murine model and compared with concurrent naïve and vehicle treated animals.

AAV5-hU6(318 bp)-SGN004282-JeT-APG05586-SV40pA was administered at 1.72E11 vg per animal (n=6) as defined by intrastriatal administration of 2 μL of 2.15E13 vg/mL per site with 2 sites per hemisphere yielding a total volume of 8 μL per mouse. Concurrent naïve (n=5) and vehicle treated animals (n=4) were utilized as controls. Brain tissues were collected at 12 weeks post injection for bioanalytical evaluation of striatal AAV5 distribution, APG05586 mRNA expression levels, human mutHTT protein levels and editing efficiency.

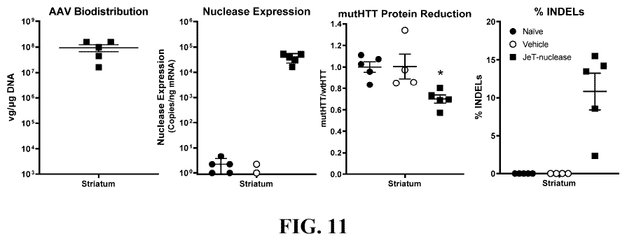

Intrastriatal administration of AAV5-hU6(318 bp)-SGN004282-JeT-APG05586-SV40pA at a dose of 1.72E11 vg was well tolerated and resulted in striatal biodistribution of 9.02×10⁷ vg/μg generating APG05586 mRNA nuclease transgene expression with 3.86×10⁴ copies/ng resulting in a statistically significant 30% decrease in mutant HTT protein compared to concurrently examined naïve animals (P<0.05) (FIG. 11). Editing was confirmed with 9.02% INDELs evident in the AAV5-hU6(318 bp)-SGN004282-JeT-APG05586 treated animals compared to 0% for both the naïve and vehicle treated animals.

### Example 5.2 Evaluation of AAV5-hU6-SGN4282-JeT-APG05586 and AAV5-hU6-SGN4282-hSyn-APG05586 to Reduce Mutant Huntingtin Protein Four Weeks Following Intrastriatal Administration in BACHD Mice

The objective of these studies was to evaluate the activity of AAV5-delivered APG05586 nuclease and single guide RNA to delete the rs362331 SNP in exon 50 of mutant huntingtin (mutHTT) protein following intrastriatal administration. Reduced mutHTT levels have been shown preclinically to halt or slow disease progression. The activity of pAAV-hU6(318 bp)-SGN004282-Jet-APG05586-SV40pA (SEQ ID NO: 32) and pAAV-hU6(318 bp)-SGN004282-hSyn-APG05586 (SEQ ID NO: 33) packaged in AAV5 was evaluated following intrastriatal administration of 1.72E11 vg and 2.84E11 respectively, in the clinically relevant BACHD murine model and compared to concurrent naïve controls.

AAV5-hU6-SGN004282-JeT-APG05586 (n=5) and AAV5-hU6-SGN004282-hSyn-APG05586 (n=6) were intrastriatally administered to BACHD mice at 1.72E11 vg and 2.84E11 vg per brain. Administration volumes consisted of 2 μL injections of 2.15E13 vg/mL for AAV5-hU6-SGN004282-Jet-APG05586 and 3.55E13 vg/mL for AAV5-hU6-SGN004282-hSyn-APG05586 with 2 sites per hemisphere yielding a total volume of 8 μL per mouse as the dose being administrated. Concurrent naïve (n=5) animals were utilized as controls. Brain tissues were collected at 4 weeks post injection for bioanalytical evaluation of striatal AAV5 distribution, APG05586 mRNA expression levels, human mutHTT protein levels and editing activity.

Intrastriatal administration of AAV5-hU6-SGN004282-JeT-APG05586 and AAV5-hU6-SGN004282-hSyn-APG05586 at doses of 1.72E11 vg and 2.84E11 vg per brain were well tolerated (FIG. 12). Extensive vector levels were present within the striatum with appreciable levels in the cortex indicating retrograde transport from the intrastriatal administration site. Striatal AAV5-hU6-SGN004282-JeT-APG05586 and AAV5-hU6-SGN004282-hSyn-APG05586 vector levels were 5.44×10⁷ and 8.41×10⁷ vg/μg, respectively with 2.85×10⁶ and 1.42×10⁷ present within the cortex corresponding to 94.8 and 83.1% percent lower levels compared to striatal levels. APG05586 nuclease transgene expression was evident with 1.38×10⁴ and 1.9×10⁴ copies/ng mRNA present within the striatum for AAV5-hU6-SGN004282-JeT-APG05586 and AAV5-hU6-SGN004282-hSyn-APG05586, respectively and 1.27×10³ and 3.77×10³ copies/ng mRNA in the cortex.

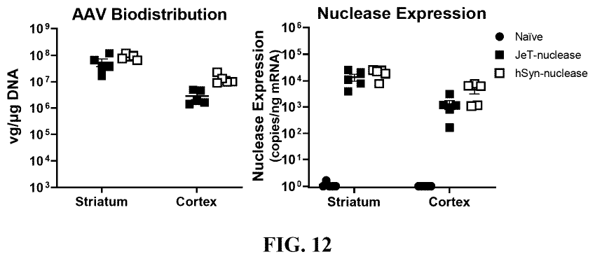

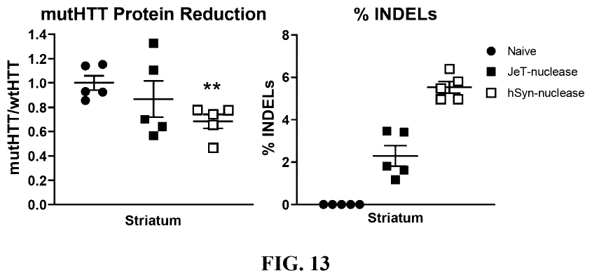

AAV5-hU6-SGN004282-JeT-APG05586 and AAV5-hU6-SGN004282-hSyn-APG05586 administration resulted in 13.3 and 31.7% reduction in mutHTT protein compared to a concurrently run naïve control cohort reaching statistical significance with the AAV5-hU6-SGN004282-hSyn-APG05586 treated animals, P<0.01 (FIG. 13). Editing was confirmed with >2% and >5% INDELs evident with AAV5-hU6-SGN004282-JeT-APG05586 and AAV5-hU6-SGN004282-hSyn-APG05586, respectively compared to 0% for concurrent naïve controls.

### Example 5.3 Evaluation of AAV5-hU6-SGN004282-hSyn-APG05586 to Reduce Mutant Huntingtin Protein Six Weeks Following Intrastriatal Administration in BACHD Mice

The objective of these studies was to evaluate the activity of AAV5-delivered APG05586 nuclease and single guide RNA to delete the rs362331 SNP in exon 50 of mutant huntingtin (mutHTT) allele following intrastriatal administration. Reduced mutHTT levels have been shown preclinically to halt or slow disease progression.

The activity of pAAV-hU6(318 bp)-SGN004282-hSyn-APG05586-SV40pA (SEQ ID NO: 33) packaged in AAV5 was evaluated 6 weeks following intrastriatal administration of 2.84E11 vg (2×2 μL per hemisphere using 3.55E13 vg/mL) in the clinically relevant BACHD murine model, n=4. Brain tissues were collected from treated animals with striatal AAV5 distribution, APG05586 mRNA expression, mutHTT mRNA and protein and editing activity levels evaluated. Concurrent vehicle treated animals (n=4) were utilized as controls.

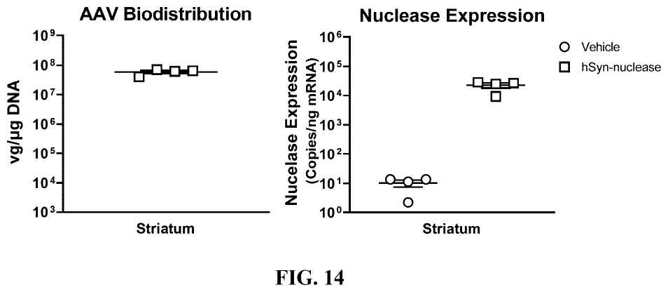

Intrastriatal administration of AAV5-hU6-SGN004282-hSyn-APG05586 at a dose of 2.84E11 vg was well tolerated resulting in striatal biodistribution of 5.97×10⁷ vg/μg and APG05586 nuclease expression of 2.25×10⁴ copies/ng mRNA (FIG. 14).

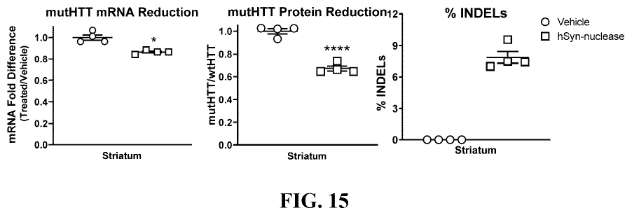

AAV5-hU6-SGN004282-hSyn-APG05586 administration generated statistically significant decreases in both mutHTT mRNA and protein reductions with 13.5% and 32.8% respectively, compared to vehicle treated concurrent controls (P<0.05 and P<0.0001) (FIG. 15). Editing was confirmed with next-generation sequencing analysis demonstrating 7.88% INDELs evident in the AAV5-hU6-SGN004282-hSyn-APG05586 treated animals compared to 0% for the concurrent vehicle treated control animals.

### Example 6. Optimization of AAV Cassette

The objective of these studies was to evaluate the activity of AAV5 cassettes expressing SGN004282 driven by the hU6 (249 bp) or hU6 (318 bp) promoter (set forth as SEQ ID NO: 128 and 89, respectively) and mammalian codon optimized APG05586 (set forth as SEQ ID NO: 88), directed by various promoters (Jet, hSyn, CMVeb, EFS; set forth as SEQ ID NOs: 92, 93, 90, and 91, respectively), in reducing mutant huntingtin following intra-striatal administration in BACHD mice.

Reduced mutHTT levels have been shown preclinically to halt or slow disease progression (Harper et al., 2005, Proc Natl Acad Sci USA 102:5820-5825; DiFiglia et al., 2007, Proc Natl Acad Sci USA 104:17204-17209; Southwell et al., 2018, Sci. Transl. Med. 10(461):10, eaar3959). As demonstrated herein, APG05586 nuclease and its concordant single guide RNA (SGN004282) reduce mutant huntingtin (mutHTT) protein in patient-derived fibroblasts by targeting the rs362331 SNP in exon 50 of the huntingtin gene. Six AAV expression cassettes containing various promoters and polyadenylation signals were engineered to optimize the expression of codon optimized APG05586 and SGN004282. In this study, all six cassettes packaged in AAV5 were delivered through intra-striatal injection in the clinically relevant BACHD murine model.

The activity of vehicle (0 vg), pAAV-hU6(318 bp)-SGN004282-JeT-APG05586mco-SV40pA (SEQ ID NO: 34) (1.26E11 vg), pAAV-hU6(318 bp)-SGN004282-hSyn-APG05586mco-SV40pA (SEQ ID NO: 35) (7.32E10 vg), pAAV-hU6(318 bp)-SGN004282-CMVeb-APG05586mco-SV40pA (SEQ ID NO: 36) (7.28E10 vg), pAAV-hU6(318 bp)-SGN004282-EFS-APG05586mco-SV40pA (SEQ ID NO: 37) (1.04E11 vg), pAAV-hU6(249 bp)-SGN004282-CMVeb-APG05586mco-bGHpA (SEQ ID NO: 38) (1.48E11 vg), and pAAV-hU6(249 bp)-SGN004282-EFS-APG05586mco-bGHpA (SEQ ID NO: 39) (1.29E11 vg) packaged in AAV5 were evaluated 6 weeks following intrastriatal administration of the Test Article (2×2 μL per hemisphere totaling 8 μL animal) in the clinically relevant BACHD murine model, n=4-5 per group. Brain tissues were collected from treated animals with striatal AAV5 distribution, mutHTT mRNA and protein and editing activity levels evaluated.

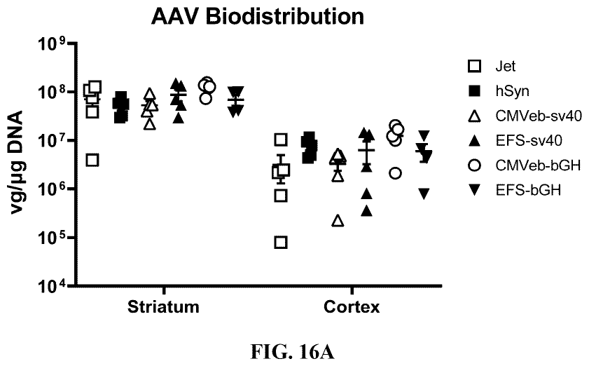

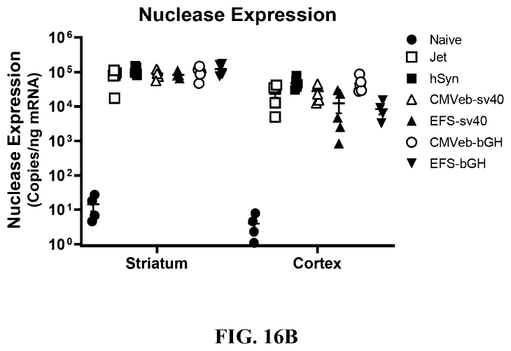

Intrastriatal injections of all vector constructs were well tolerated. AAV5 vector disposition in the striata of the animals treated with AAV5-JeT-APG05586cmo, AAV5-hSyn-APG05586cmo, AAV5-CMVeb-sv40, AAV5-EFS-sv40, AAV5-CMVeb-bGH, and AAV5-EFS-bGH were 7.07×10⁷, 5.13×10⁷, 5.29×10⁷, 8.73×10⁷, 1.22×10⁸, and 6.93×10⁷ respectively with corresponding cortex levels of 3.15×10⁶, 7.61×10⁶, 3.32×10⁶, 6.33×10⁶, 1.23×10⁷, 6.01×10⁶ vg/μg relating to 95.5, 86.2, 93.7, 92.7, 89.9, and 91.3 percent lower levels compared to striatal levels and implying the likelihood of retrograde transport (FIG. 16A). Concurrently run vehicle control animals were below the limit of quantification.

Nuclease expression for AAV5-JeT-APG05586cmo, AAV5-hSyn-APG05586cmo, AAV5-CMVeb-sv40, AAV5-EFS-sv40, AAV5-CMVeb-bGH, and AAV5-EFS-bGH were 7.73×10⁴, 1.19×10⁵, 9.02×10⁴′ 8.38×10⁴, 9.99×10⁴ and 1.24×10⁵ copies/ng mRNA respectively with corresponding cortex levels of 2.51×10⁴, 4.89×10⁴, 2.79×10⁴, 1.22×10⁴, 4.74×10⁴, and 8.37×10³ copies/ng respectively (FIG. 16B).

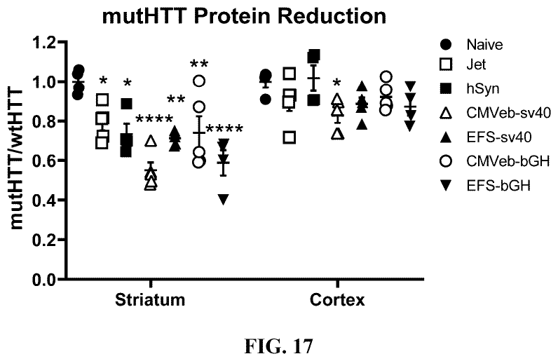

Substantial human mutant HTT protein reduction was observed with all vector constructs in the striatum when evaluated 6 weeks post intrastriatal administration (FIG. 17). Reductions in human mutHTT of 21.0, 26.5, 44.9, 28.6, 25.8, and 41.1% occurred with AAV5-JeT-APG05586cmo, AAV5-hSyn-APG05586mco, AAV5-CMVeb-APG05586mco-sv40, AAV5-EFS-APG05586mco-sv40, AAV5-CMVeb-APG05586mco-bGH, and AAV5-EFS-APG05586mco-bGH, respectively. Mutant HTT protein reductions were also observed in the cortex with all constructs except AAV5-hSyn-APG05586mco. Percent protein reductions of 9.7, 17.1, 11.3, 7.8, and 12.7% occurred for AAV5-JeT-APG05586cmo, AAV5-CMVeb-APG05586mco-sv40, AAV5-EFS-APG05586mco-sv40, AAV5-CMVeb-APG05586mco-bGH, and AAV5-EFS-APG05586mco-bGH. AAV5-CMVeb-APG05586mco-sv40 was the only construct to exhibit a statistically significant reduction in human mutHTT in the cortex (P<0.05).

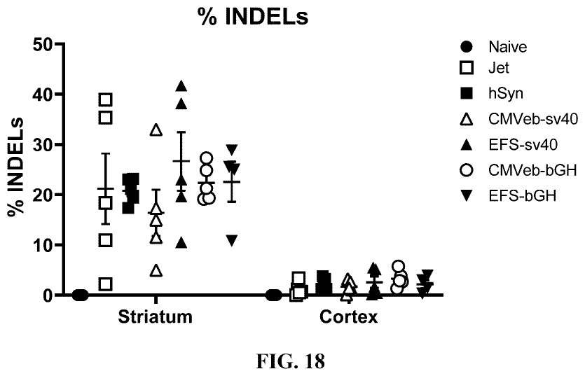

Editing was confirmed using NGS with INDELs observed with all vector constructs within the striatum: 21.1, 20.8, 16.4, 26.6, 22.3, and 22.5% for AAV5-JeT-APG05586mco, AAV5-hSyn-APG05586mco, AAV5-CMVeb-APG05586mco-sv40, AAV5-EFS-APG05586mco-sv40, AAV5-CMVeb-APG05586mco-bGH, and AAV5-EFS-APG05586mco-bGH, respectively, with corresponding lower levels of 1.15, 2.23, 1.69, 2.57, 3.27, and 2.17% detected in the cortex (FIG. 18). No INDELs were detected in the striata or cortices of naïve animals.

### Example 7: Effect of Low, Medium, and High Doses of AAV5-hU6-SGN004282-hSyn-APG05586Mco-SV40pA in Murine Model

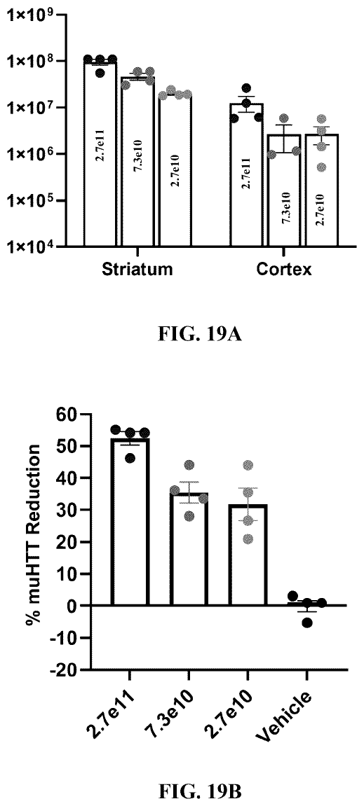

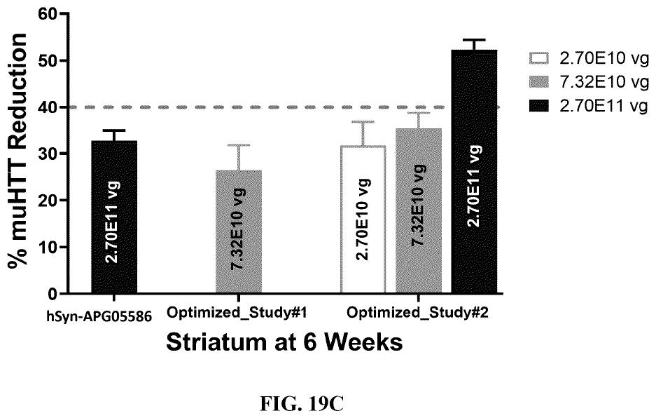

The activity of vehicle (0 vg) and three different doses of pAAV-hU6(318 bp)-SGN004282-hSyn-APG05586mco-SV40pA (SEQ ID NO: 35) (2.7E10 vg, 7.3E10 vg, and 2.7E11 vg) packaged in AAV5 were evaluated 6 weeks following intrastriatal administration of the test Article (2×2 μL per hemisphere totaling 8 μL animal) in the clinically relevant BACHD murine model, n=4-5 per group. Brain tissues were collected from treated animals with striatal AAV5 distribution, mutHTT mRNA and protein and editing activity levels evaluated. Results are shown in FIGS. 19A-19C. A clinically relevant reduction of mutHTT protein, defined as a 40% reduction, was observed in BACHD striatum after 6 weeks with AAV5-hU6-SGN004282-hSyn-APG05586mco-SV40pA at a dose of 2.7e11 vg/animal.

### Example 8: Treatment of Huntington's Disease in Humans

Huntington's Disease is a fatal genetic disorder characterized by progressive motor dysfunction, psychiatric disorders and cognitive decline. Huntington's Disease patients are screened for the number of CAG repeats in the HTT gene and the identification of the rs362331 SNP in exon 50 in phase with heterozygosity. In addition, a volumetric MRI may be used to image the brain of the patient and visualize the putamen and/or caudate volume. After administration of the construct, a functional MRI may be used to assess the prevention of or delay of a further reduction in volume of brain structures, such as putamen and/or caudate volume. Other biomarkers of pathogenesis that may be assessed include levels of wild type HTT mRNA and/or protein, levels of mutant HTT mRNA and/or protein, or levels of neurofilament light chain (NFL) protein. Levels of NFL protein are known to increase in cerebrospinal fluid (CSF) proportionally to the degree of neuronal damage, and a decrease in these levels could be used to assess beneficial effects of treatment. Finally, patients are screened for physical symptoms and functional change.

For this first-in-human study, being mindful of the ethical and technical aspects of delivery and benefit:risk in this patient population, Huntington's Disease patients classified as HD-ISS “Stage 2”” (with CAP100 enrichment) to early-stage 3 with Total Functional capacity (TFC) of 11+ is proposed (e.g. those having ≥40 CAG repeats, biomarkers of pathogenesis, signs or symptoms, and functional change, for example, as measured by the Independence Scale and/or the Total Functional Capacity (TFC)) to be selected for treatment. Patients are anesthetized and an AAV-delivered construct comprising nucleotide sequences encoding SEQ ID NO: 7 and SEQ ID NOs: 25 or 26 are administered to the striatum. Suggested outcome measures, including a totality of evidence, could entail volumetric brain imaging, PET striatum imaging, CSF mHTT, CSF and plasma neurofilament for safety and activity, cognitive assessments (e.g, symbol digit modality test, Stroop word test, and verbal fluence), motor assessments (e.g, total motor score), functional assessments (e.g., total functional capacity test), and digital measures. Beginning 4 weeks post-administration, biomarkers of pathogenesis may be assessed to determine if gene editing at the mutant HTT allele has occurred.

### Example 9. Capillary Electrophoresis Immunoassay for the Detection and Quantification of Mutant HTT and Wild Type HTT Protein

Capillary electrophoresis (CE) allows for the separation of proteins based on molecular size. Briefly, under the influence of an electric field, proteins migrate through a hydrophilic polymer which has a sieving effect with larger proteins migrating more slowly.

Samples are heat denatured in the presence of sodium dodecyl sulfate (SDS). These treated proteins are separated through an SDS-containing polymer matrix in a capillary under a voltage differential. Following separation of the proteins according to their molecular weights, they are covalently linked to the capillary. The proteins are then treated with primary antibodies reactive to the protein of interest, followed by secondary antibody conjugated with HRP. Finally, HRP substrate is applied, and a luminescent signal is quantified.

Most biological samples to be analyzed by this technique will contain the HTT wild type protein as well as mutant forms. Simultaneous quantification of these proteins is therefore a key requirement. The excellent resolution of capillary electrophoresis and the powerful peak integration software applied to the CE Western program allows simultaneous integration of both the wild-type and mutant HTT peaks in the same sample using one HTT-specific antibody.

The method established a range of Calibration Standard concentrations of Q73 HTT and Q7 HTT from 30.0 ng/ml to 0.12 ng/ml by serial dilutions of 30.0 ng/ml of Q73 HTT Calibration Stock Solution (CH0326, Coriell) or Q7 HTT Calibration Stock Solution (CH03222, Coriell) in 0.1× Jess sample buffer.

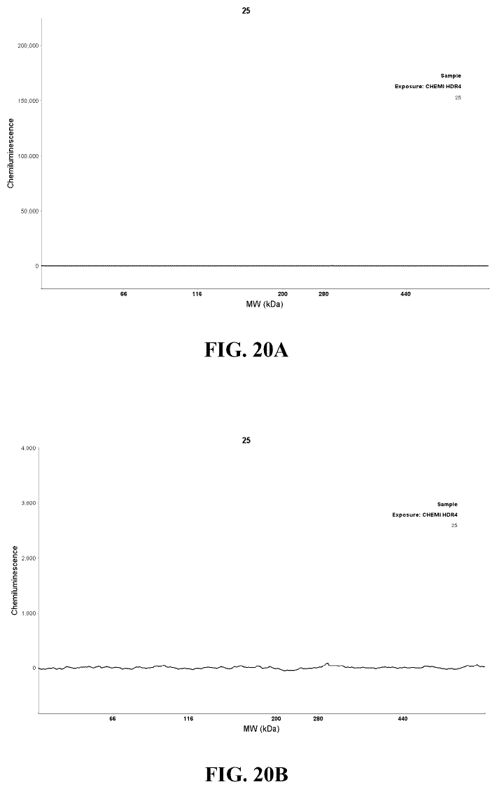

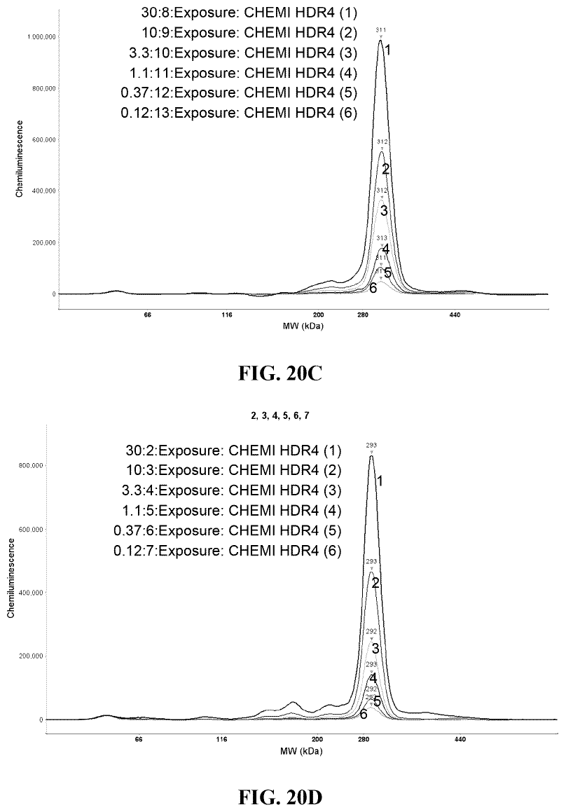

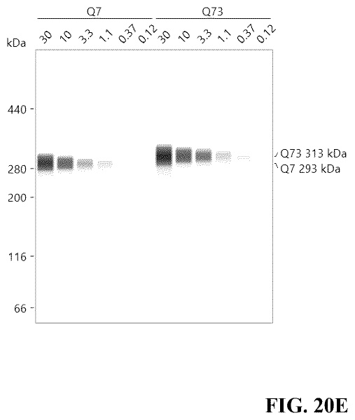

The specificity of the assay was determined by analyzing 0.1× Jess sample buffer without added Calibration Standards. The blank did not have interfering peaks in the molecular weight region of interest in the electropherograms of Q73HTT and Q7HTT, demonstrating acceptable specificity (see FIGS. 20A-20E).

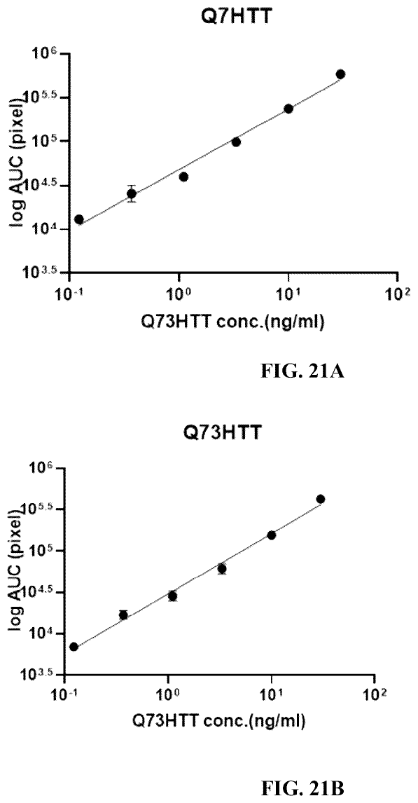

To verify linearity of the assay, size Calibration Standards ranging from 30.0-0.12 ng/mL for Q73HTT and Q7HTT were tested (see FIGS. 21A and 21B and Tables 24 and 25). These results meet the typical acceptance criterion of R²≥0.95.

The precision of the CE-Western method was evaluated using % RSDs of the average apparent molecular weight and area under the curve (AUC) for Q73HTT (Table 26) and Q7HTT (Table 27). For Q73HTT, the % RSDs of the apparent molecular weight was <0.20% while the % RSDs for AUC <8%. For Q7HTT, the % RSDs for the apparent molecular weight was <0.25% while the % RSDs for AUC was <10%. Both the apparent molecular weights and AUCs are within the generally accepted criteria of <10%.

To further evaluate the method, % recovery was calculated from the AUCs of the linearity samples. Accuracy was demonstrated by comparing the concentration of the Calibration Standard to the calculated amount based on interpolation of the calibration line. Recovery of mutHTT ranged from 87% to 113%; wtHTT ranged from 81% to 126% (Tables 28 and 29). The recovery for both HTT species is within the typical assay acceptance of 70-130%.

In conclusion, the method was able to measure wtHTT and mutHTT with specificity, linearity, precision, and accuracy. Linearity can be observed in a wide range (˜50 μg/ml to 10 ng/ml). Accuracy which was determined by a comparison of theoretical to calculated concentrations yielded a recovery of 87% to 113%. Precision, which was evaluated by comparison of the % RSD for the average MW and average peak size in pixels was 9-20% and 1.30-6.88% respectively, and the back-calculated concentration recoveries were generally greater than 90%. This method is sensitive with a LLOQ of 50 μg/ml with a S/N of 17.

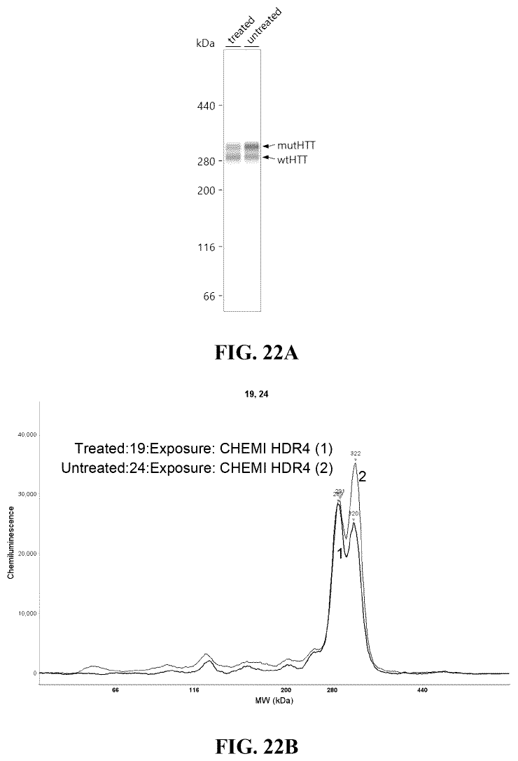

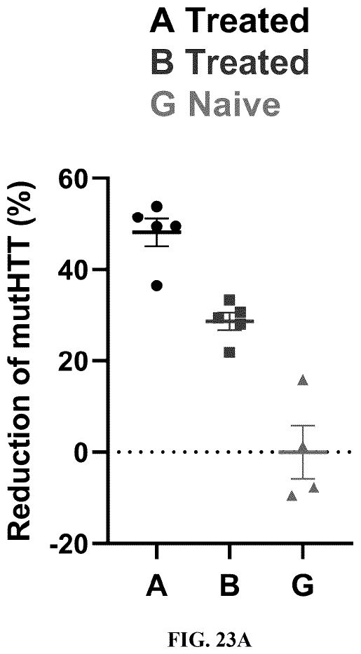

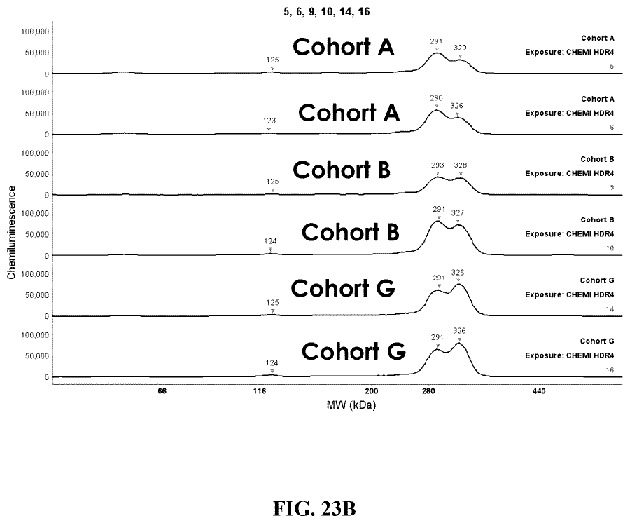

Overall, these results demonstrate this method has successfully been developed and can be implemented for sample analysis and is suitable for qualification. As a test sample for this method, BACHD brain samples from mice treated with an AAV5 construct comprising SGN004282 under control of a hU6 promoter and a codon-optimized APG05586 under control of a CMV promoter (Cohort A and Treatment 1) or an EFS promoter (Cohort B and Treatment 2) and naïve samples were used to assess the ratio of mutHTT to wtHTT. In this mouse model, mutHTT from the human contains five copies of 98 polyQ while the endogenous mouse wtHTT has 2 copies of 15 polyQ. The protein ratio of mutHTT/wtHTT is about 1.25. FIGS. 22A and 22B indicate that mutHTT and wtHTT are separated based on their molecular weights and their AUC ratio of mutHTT/wtHTT is shown in Table 30. FIGS. 23A and 23B show a reduction in mutHTT levels in BACHD mice treated with both constructs.

TABLE 24
Summary of Q73HTT linearity

| | | Nominal | | Back- |
|---|---|---|---|---|
| | | HCP | | Calculated |
| Linearity | | Concen- | | Concen- |
| Preparation | Sample | tration | RLU | tration |
| # | Description | (ng/mL) | at Pixels | (ng/mL) |
| Prep 1 | 30 ng/mL | 30.0 | 442,506 | 35.4 |
| | Standard 1 | | | |
| | 10 ng/mL | 10.0 | 161,938 | 9.21 |
| | Standard 1 | | | |
| | 3.3 ng/mL | 3.33 | 68,008 | 2.88 |
| | Standard 1 | | | |
| | 1.1 ng/mL | 1.11 | 31,354 | 1.02 |
| | Standard 1 | | | |
| | 0.37 ng/mL | 0.37 | 15,471 | 0.396 |
| | Standard 1 | | | |
| | 0.12 ng/mL | 0.12 | 6,802 | 0.131 |
| | Standard 1 | | | |
| Prep 2 | 30 ng/mL | 30.0 | 412,811 | 32.3 |
| | Standard 2 | | | |
| | 10 ng/mL | 10.0 | 149,353 | 8.26 |
| | Standard 2 | | | |
| | 3.3 ng/mL | 3.33 | 55,940 | 2.22 |
| | Standard 2 | | | |
| | 1.1 ng/mL | 1.11 | 25,856 | 0.787 |
| | Standard 2 | | | |
| | 0.37 ng/mL | 0.37 | 18,439 | 0.500 |
| | Standard 2 | | | |
| | 0.12 ng/mL | 0.12 | 7,098 | 0.139 |
| | Standard 2 | | | |
| | Intercept | 4.48 | | |
| | Slope | 0.73 | | |
| | R² | 0.99 | | |

TABLE 25
Summary of Q7HTT linearity

| | | Nominal | | Back- |
|---|---|---|---|---|
| | | HCP | | Calculated |
| Linearity | | Concen- | | Concen- |
| Preparation | Sample | tration | RLU | tration |
| # | Description | (ng/mL) | at Pixels | (ng/mL) |
| Prep 1 | 30 ng/mL Standard 1 | 30.0 | 442,506 | 35.4 |
| | 10 ng/mL Standard 1 | 10.0 | 161,938 | 9.21 |
| | 3.3 ng/mL Standard 1 | 3.33 | 68,008 | 2.88 |
| | 1.1 ng/mL Standard 1 | 1.11 | 31,354 | 1.02 |
| | 0.37 ng/mL Standard 1 | 0.37 | 15,471 | 0.396 |
| | 0.12 ng/mL Standard 1 | 0.12 | 6,802 | 0.131 |
| Prep 2 | 30 ng/mL Standard 2 | 30.0 | 412,811 | 32.3 |
| | 10 ng/mL Standard 2 | 10.0 | 149,353 | 8.26 |
| | 3.3 ng/mL Standard 2 | 3.33 | 55,940 | 2.22 |
| | 1.1 ng/mL Standard 2 | 1.11 | 25,856 | 0.787 |
| | 0.37 ng/mL Standard 2 | 0.37 | 18,439 | 0.500 |
| | 0.12 ng/mL Standard 2 | 0.12 | 7,098 | 0.139 |
| | Intercept | 4.48 | | |
| | Slope | 0.71 | | |
| | R² | 0.98 | | |

TABLE 26
Summary of Q73HTT precision.

| | | | Mean | STD | % RSD | | Mean | | |
|---|---|---|---|---|---|---|---|---|---|
| Nominal | | Apparent | Apparent | Dev of | of | Back- | Back- | | |
| Q73 | | Molecular | Molecular | Apparent | Apparent | Calculated | Calculated | STD | % RSD |
| Concentration | | Weight | Weight | Molecular | Molecular | concentration | Concentration | Dev of | of |
| (ng/ml) | Prep # | (kDa) | (kDa) | Weight | Weight | (ng/ml) | (ng/ml) | Concentration | Concentration |
| 30 | 1 | 311 | 308 | 0.71 | 0.00% | 35.4 | 33.86 | 2.23 | 6.6% |
| | 2 | 311 | | | | 32.3 | | | |
| 10 | 1 | 312 | 308 | 0.00 | 0.00% | 9.21 | 8.74 | 0.669 | 7.7% |
| | 2 | 312 | | | | 8.26 | | | |
| 3.3 | 1 | 313 | 306 | 0.71 | 0.20% | 2.88 | 3.05 | 0.238 | 7.8% |
| | 2 | 312 | | | | 3.22 | | | |
| 1.1 | 1 | 311 | 296 | 2.12 | 0.00% | 1.02 | 1.00 | 0.023 | 2.3% |
| | 2 | 311 | | | | 0.987 | | | |
| 0.37 | 1 | 311 | 289 | 2.12 | 0.00% | 0.396 | 0.40 | 0.003 | 0.8% |
| | 2 | 311 | | | | 0.400 | | | |
| 0.12 | 1 | 311 | 287 | 0.00 | 0.00% | 0.131 | 0.14 | 0.005 | 4.0% |
| | 2 | 311 | | | | 0.139 | | | |

TABLE 27
Summary of Q7HTT precision.

| | | | Mean | STD | | | | | |
|---|---|---|---|---|---|---|---|---|---|
| | | Apparent | Apparent | Dev of | % RSD of | Back- | Mean Back- | | |
| Nominal Q7 | | Molecular | Molecular | Apparent | Apparent | Calculated | Calculated | | |
| Concentration | | Weight | Weight | Molecular | Molecular | Concentration | Concentration | STD Dev of | % RSD of |
| (ng/ml) | Prep # | (kDa) | (kDa) | Weight | Weight | (ng/ml) | (ng/ml) | Concentration | Concentration |
| 30 | 1 | 293 | 287 | 0.00 | 0.00% | 37.340 | 36.25 | 1.54 | 4.2% |
| | 2 | 293 | | | | 35.166 | | | |
| 10 | 1 | 293 | 289 | 0.707 | 0.25% | 10.377 | 10.13 | 0.35 | 3.5% |
| | 2 | 292 | | | | 9.878 | | | |
| 3.3 | 1 | 293 | 286 | 1.414 | 0.00% | 3.375 | 3.30 | 0.10 | 3.1% |
| | 2 | 293 | | | | 3.229 | | | |
| 1.1 | 1 | 292 | 284 | 0.000 | 0.00% | 0.947 | 0.89 | 0.09 | 9.8% |
| | 2 | 292 | | | | 0.824 | | | |
| 0.37 | 1 | 292 | 285 | 2.121 | 0.25% | 0.316 | 0.33 | 0.03 | 7.8% |
| | 2 | 293 | | | | 0.353 | | | |
| 0.12 | 1 | 292 | 286 | 0.707 | 0.00% | 0.147 | 0.15 | 0.01 | 4.2% |
| | 2 | 292 | | | | 0.156 | | | |

TABLE 28
Summary of Q73HTT accuracy

| | | Back- | Mean Back- | | | |
|---|---|---|---|---|---|---|
| Nominal Q73 | | Calculated | Calculated | | | |
| Concentration | Preparation | Concentration | Concentration | STD Dev of | % | Mean % |
| (ng/ml) | # | (ng/ml) | (ng/ml) | Concentration | Recovery | Recovery |
| 30 | 1 | 35.4 | 33.86 | 2.23 | 118.1% | 112.9% |
| | 2 | 32.3 | | | 107.6% | |
| 10 | 1 | 9.21 | 8.74 | 0.669 | 92.1% | 87.4% |
| | 2 | 8.26 | | | 82.6% | |
| 3.3 | 1 | 2.88 | 3.05 | 0.238 | 87.2% | 92.3% |
| | 2 | 3.22 | | | 97.4% | |
| 1.1 | 1 | 1.02 | 1.00 | 0.023 | 92.7% | 91.2% |
| | 2 | 0.987 | | | 89.8% | |
| 0.37 | 1 | 0.396 | 0.40 | 0.003 | 106.9% | 107.5% |
| | 2 | 0.400 | | | 108.1% | |
| 0.12 | 1 | 0.131 | 0.14 | 0.005 | 109.6% | 112.8% |
| | 2 | 0.139 | | | 116.0% | |

TABLE 29
Summary of Q7HTT accuracy

| | | Back- | Mean Back- | | | |
|---|---|---|---|---|---|---|
| Nominal Q7 | | Calculated | Calculated | | | |
| Concentration | Preparation | Concentration | Concentration | STD Dev of | % | Mean % |
| (ng/ml) | # | (ng/ml) | (ng/ml) | Concentration | Recovery | Recovery |
| 30 | 1 | 37.340 | 36.25 | 1.54 | 124.5% | 120.8% |
| | 2 | 35.166 | | | 117.2% | |
| 10 | 1 | 10.377 | 10.13 | 0.35 | 103.8% | 101.3% |
| | 2 | 9.878 | | | 98.8% | |
| 3.3 | 1 | 3.375 | 3.30 | 0.10 | 102.3% | 100.1% |
| | 2 | 3.229 | | | 97.9% | |
| 1.1 | 1 | 0.947 | 0.89 | 0.09 | 86.1% | 80.5% |
| | 2 | 0.824 | | | 74.9% | |
| 0.37 | 1 | 0.316 | 0.33 | 0.03 | 85.4% | 90.4% |
| | 2 | 0.353 | | | 95.3% | |
| 0.12 | 1 | 0.147 | 0.15 | 0.01 | 122.5% | 126.3% |
| | 2 | 0.156 | | | 130.0% | |

TABLE 30
Area under the curve for BACHD brain sample.

| Sample | Cap | MW (kDa) | Height | Area | Width | S/N | mutHTT/wtHTT |
|---|---|---|---|---|---|---|---|
| Treated | 19 | 289 | 26908.3 | 455516.9 | 15.9 | 889.3 | |
| Treated | 19 | 320 | 23509 | 387122.5 | 15.5 | 776.9 | 0.850 |
| Untreated | 24 | 291 | 26866.6 | 452046.8 | 16.6 | 1343.1 | |
| Untreated | 24 | 322 | 32899.8 | 557894 | 13.8 | 1644.8 | 1.23 |

### Example 10: Effect of Low, Medium, and High Doses of AAV5-hU6-SGN004282-CMVeb-APG05586Mco-SV40pA and AAV5-hU6-SGN004282-EFS-APG05586Mco-bGHpA in Murine Model

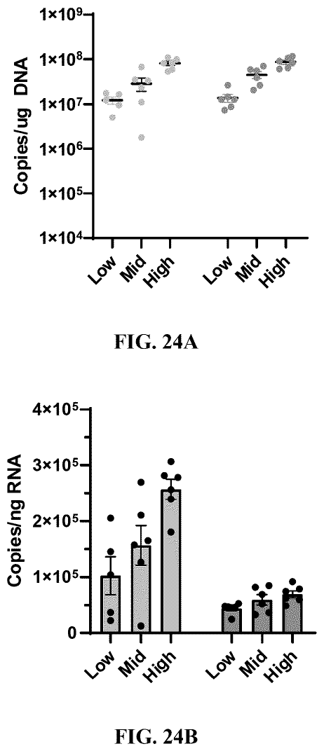

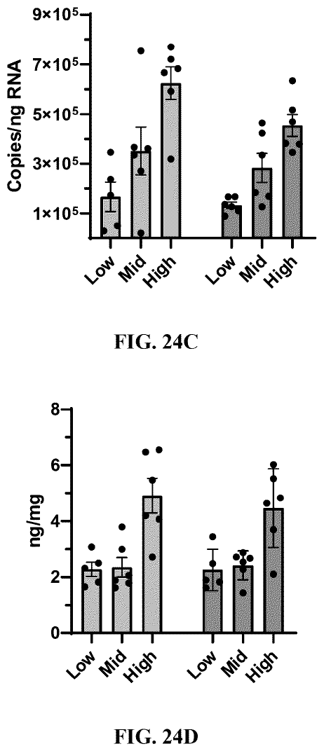

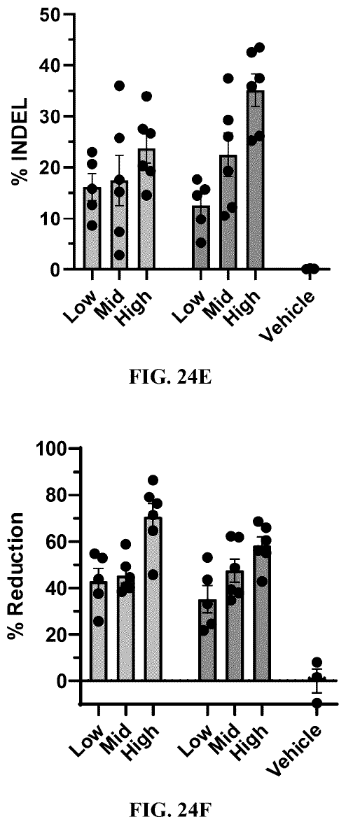

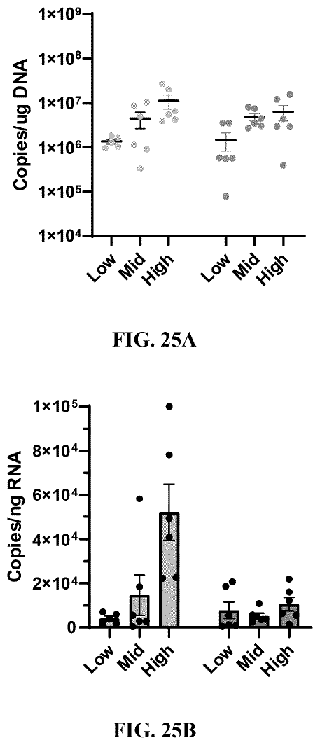

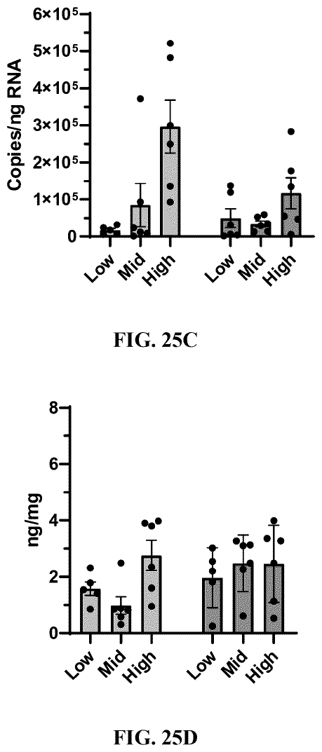

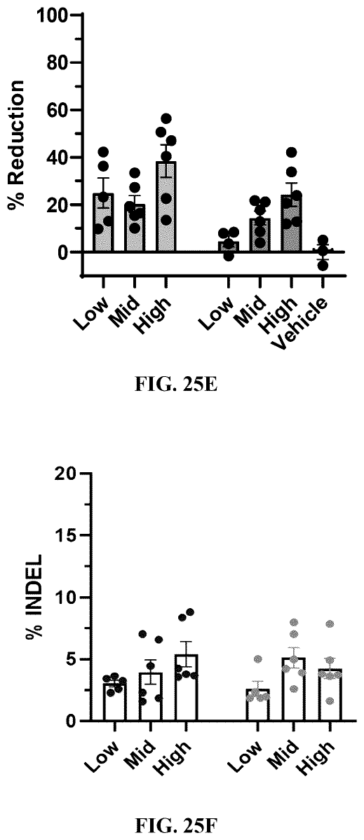

The activity of vehicle (0 vg), three different doses of pAAV-hU6(318 bp)-SGN004282-CMVeb-APG05586mco-SV40pA (SEQ ID NO: 36) (2.05E10 vg, 7.28E10 vg, and 2.94E11 vg), and three different doses of pAAV-hU6(249 bp)-SGN004282-EFS-APG05586mco-bGHpA (SEQ ID NO: 39) (2.05E10 vg, 7.28E10 vg, and 2.05E11 vg) packaged in AAV5 were evaluated 12 weeks following intrastriatal administration of the test Article (2×2 μL per hemisphere totaling 8 μL animal) in the clinically relevant BACHD murine model, n=5-6 per group. Brain tissues were collected from treated animals with striatal and cortical AAV5 distribution, mutHTT mRNA and protein and editing activity levels evaluated. Striatal results are shown in FIGS. 24A-24F and cortical results in FIGS. 25A-25F. A clinically relevant reduction of mutHTT protein was observed in BACHD striatum after 12 weeks with both constructs at both the mid and high doses (7.28E10 vg and 2.94E11 vg of AAV5-hU6-SGN004282-CMVeb-APG05586mco-SV40pA and 7.28E10 vg and 2.05E11 bg of AAV5-hU6-SGN004282-EFS-APG05586mco-bGHpA), which correlated with an observed dose-dependent increase in INDELs.

### Example 11: Optimization of AAV Cassettes in the BACHD Murine Model

The objective of these studies was to evaluate the activity of three novel AAV cassette designs, expressing SGN004282 driven by the hU6 (249 bp) or hU6 (318 bp) promoter (set forth as SEQ ID NOs: 128 and 89, respectively) and mammalian codon optimized APG05586 (APG05586mco; set forth as SEQ ID NO: 88) directed by the CMVeb (225 bp) promoter (set forth as SEQ ID NO: 90), in editing the genomic target HTT exon50 rs362331 and in reducing mutant huntingtin protein following intra-striatal administration in BACHD mice.

Reduced mutHTT levels have been shown preclinically to halt or slow disease progression (Harper et al., 2005, Proc Natl Acad Sci USA 102:5820-5825; DiFiglia et al., 2007, Proc Natl Acad Sci USA 104:17204-17209; Southwell et al., 2018, Sci. Transl. Med. 10(461):10, eaar3959). As demonstrated herein, APG05586mco nuclease and its concordant single guide RNA (SGN004282) reduce mutant huntingtin (mutHTT) protein in patient-derived fibroblasts by targeting the rs362331 SNP in exon 50 of the Huntingtin gene. Three AAV expression cassettes were engineered to optimize the activity of APG05586mco and SGN004282 by reducing the total AAV cassette size, utilizing different promoters to drive expression of SGN004282, utilizing different nuclear localization signals (NLSs) to improve translocation of APG00586mco, or a combination thereof. The parental construct (set forth as SEQ ID NO: 36) includes an SV40 NLS, set forth as SEQ ID NO: 86, at the N-terminus of APG05586mco, and a nucleoplasmin (NP) NLS, set forth as SEQ ID NO: 87, at the C-terminus of APG05586mco. In contrast, one of the new cassettes, set forth as SEQ ID NO: 123, utilizes a c-MYC NLS, set forth as SEQ ID NO: 125, at both the N- and C-termini of APG05586mco, wherein the NLS and nuclease sequences are separated by a NLS linker (set forth as SEQ ID NO: 127). The parental construct (set forth as SEQ ID NO: 36) comprises an hU6(318 bp) promoter sequence (set forth as SEQ ID NO: 89) that regulates the expression of the SGN004282 guide RNA, whereas two of the new cassettes, set forth as SEQ ID NOs: 122 and 123, utilize a shorter hU6(249 bp) promoter sequence, which is set forth as SEQ ID NO: 128. In this study, all three new cassettes (and the parental construct) were packaged into an AAV5 viral vector and were delivered through intra-striatal injection in the clinically relevant BACHD murine model.

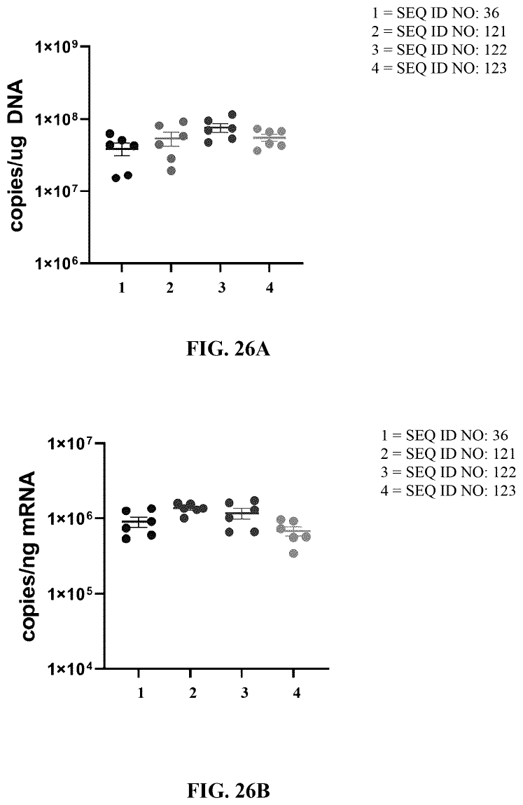

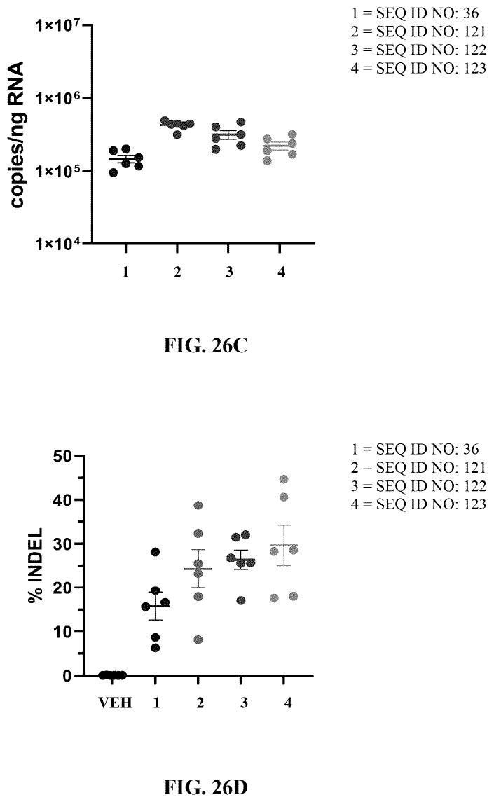

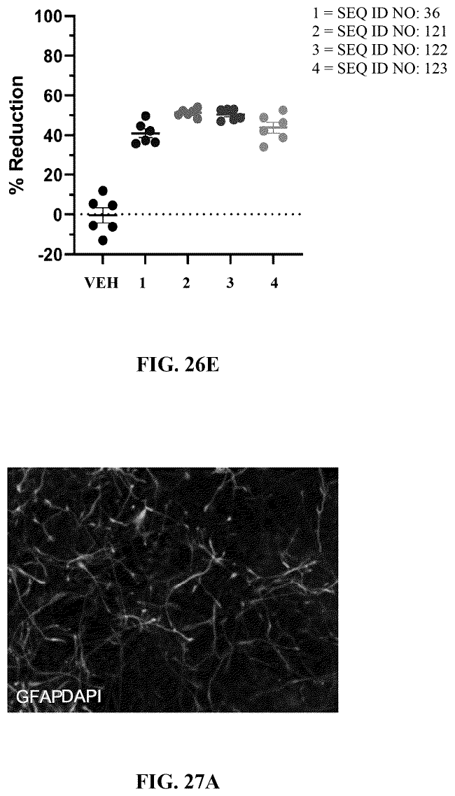

The activity of vehicle (0 vg), pAAV-hU6(318 bp)-SGN004282-CMVeb-SV40-NLS-APG05586mco-NP-NPS-SV40pA(179 bp) (SEQ ID NO: 36) (7.28E10 vg) packaged in AAV5, pAAV-hU6(318 bp)-SGN004282-CMVeb-SV40-NLS-APG05586mco-NP-NLS-SV40pA(179 bp)-MINIMIZED in which sequences between elements were removed (SEQ ID NO: 121) (7.17E10 vg) packaged in AAV5, pAAV-hU6(249 bp)-SGN004282-CMVeb-SV40-NLS-APG05586mco-NP-NLS-SV40pA(179 bp) (SEQ ID NO: 122) (7.28E10 vg) packaged in AAV5, or pAAV-hU6(249 bp)-SGN004282-CMVeb-c-MYC-NLS-APG05586mco-c-MYC-NLS-SV40pA(179 bp) (SEQ ID NO: 123) (7.28E10 vg) packaged in AAV5, were evaluated 6 weeks following intrastriatal administration of the test article (2×2 μL per hemisphere totaling 8 μL animal) in the clinically relevant BACHD murine model, n=6 per group. Striatal brain tissue was collected from treated animals for evaluation of AAV5 vector genomes, APG05586mco mRNA expression, SGN004282 RNA expression, editing of the target HTT gene, and mutant HTT protein levels. Intrastriatal injections of AAV vectors was well tolerated. Results from striatal tissue are shown in FIGS. 26A-26E.

AAV5 vector disposition in the striata of the animals treated with AAV5-packaged pAAV-hU6(318 bp)-SGN004282-CMVeb-SV40-NLS-APG05586mco-NP-NPS-SV40pA(179 bp) (SEQ ID NO:36), AAV5-packaged pAAV-hU6(318 bp)-SGN004282-CMVeb-SV40-NLS-APG05586mco-NP-NLS-SV40pA(179 bp)-MINIMIZED (SEQ ID NO: 121), AAV5-packaged pAAV-hU6(249 bp)-SGN004282-CMVeb-SV40-NLS-APG05586mco-NP-NLS-SV40pA(179 bp) (SEQ ID NO: 122), or AAV5-packaged pAAV-hU6(249 bp)-SGN004282-CMVeb-c-MYC-NLS-APG05586mco-c-MYC-NLS-SV40pA(179 bp) (SEQ ID NO: 123) were 3.86E07, 5.38E07, 7.58E07, and 5.52E07 copies/μL DNA, respectively (FIG. 26A). Concurrently run vehicle control animals were below the limit of quantification.

APG05586mco nuclease expression in the striata of the animals treated with AAV5-packaged pAAV-hU6(318 bp)-SGN004282-CMVeb-SV40-NLS-APG05586mco-NP-NPS-SV40pA(179 bp) (SEQ ID NO:36), AAV5-packaged pAAV-hU6(318 bp)-SGN004282-CMVeb-SV40-NLS-APG05586mco-NP-NLS-SV40pA(179 bp)-MINIMIZED (SEQ ID NO: 121), AAV5-packaged pAAV-hU6(249 bp)-SGN004282-CMVeb-SV40-NLS-APG05586mco-NP-NLS-SV40pA(179 bp) (SEQ ID NO: 122), or AAV5-packaged pAAV-hU6(249 bp)-SGN004282-CMVeb-c-MYC-NLS-APG05586mco-c-MYC-NLS-SV40pA(179 bp) (SEQ ID NO: 123) were 9.01E05, 1.36E06, 1.16E06, and 6.79E05 copies/ng mRNA, respectively (FIG. 26B). Concurrently run vehicle control animals were below the limit of quantification.

SGN004282 guide RNA expression in the striata of the animals treated with AAV5-packaged pAAV-hU6(318 bp)-SGN004282-CMVeb-SV40-NLS-APG05586mco-NP-NPS-SV40pA(179 bp) (SEQ ID NO:36), AAV5-packaged pAAV-hU6(318 bp)-SGN004282-CMVeb-SV40-NLS-APG05586mco-NP-NLS-SV40pA(179 bp)-MINIMIZED (SEQ ID NO: 121), AAV5-packaged pAAV-hU6(249 bp)-SGN004282-CMVeb-SV40-NLS-APG05586mco-NP-NLS-SV40pA(179 bp) (SEQ ID NO: 122), or AAV5-packaged pAAV-hU6(249 bp)-SGN004282-CMVeb-c-MYC-NLS-APG05586mco-c-MYC-NLS-SV40pA(179 bp) (SEQ ID NO: 123) were 1.47E05, 4.25E05, 3.14E05, and 2.21E05 copies/ng RNA, respectively (FIG. 26C). Concurrently run vehicle control animals were below the limit of quantification.

Editing of the target genomic locus was confirmed using next generation sequencing (NGS) with insertions-deletions (INDELs) observed with all vector constructs within the striatum. INDELs in the striata of the animals treated with AAV5-packaged pAAV-hU6(318 bp)-SGN004282-CMVeb-SV40-NLS-APG05586mco-NP-NPS-SV40pA(179 bp) (SEQ ID NO:36), AAV5-packaged pAAV-hU6(318 bp)-SGN004282-CMVeb-SV40-NLS-APG05586mco-NP-NLS-SV40pA(179 bp)-MINIMIZED (SEQ ID NO: 121), AAV5-packaged pAAV-hU6(249 bp)-SGN004282-CMVeb-SV40-NLS-APG05586mco-NP-NLS-SV40pA(179 bp) (SEQ ID NO: 122), or AAV5-packaged pAAV-hU6(249 bp)-SGN004282-CMVeb-c-MYC-NLS-APG05586mco-c-MYC-NLS-SV40pA(179 bp) (SEQ ID NO: 123) were 15.8, 24.3, 26.4, and 29.6%, respectively (FIG. 26D). Concurrently run vehicle control animals had INDEL rates <0.01% in striata samples.

A clinically relevant reduction of mutHTT protein was observed in BACHD striata after 6 weeks with all AAV construct designs. Reductions in human mutHTT in the striata of animals treated with AAV5-packaged pAAV-hU6(318 bp)-SGN004282-CMVeb-SV40-NLS-APG05586mco-NP-NPS-SV40pA(179 bp) (SEQ ID NO:36), AAV5-packaged pAAV-hU6(318 bp)-SGN004282-CMVeb-SV40-NLS-APG05586mco-NP-NLS-SV40pA(179 bp)-MINIMIZED (SEQ ID NO: 121), AAV5-packaged pAAV-hU6(249 bp)-SGN004282-CMVeb-SV40-NLS-APG05586mco-NP-NLS-SV40pA(179 bp) (SEQ ID NO: 122), or AAV5-packaged pAAV-hU6(249 bp)-SGN004282-CMVeb-c-MYC-NLS-APG05586mco-c-MYC-NLS-SV40pA(179 bp) (SEQ ID NO: 123) were 41.0, 51.2, 50.3, and 43.8%, respectively (FIG. 26E). Concurrently run vehicle control animals showed no reduction in mutHTT protein levels.

### Example 12: Optimization of AAV Cassettes and Validation with In Vitro Models

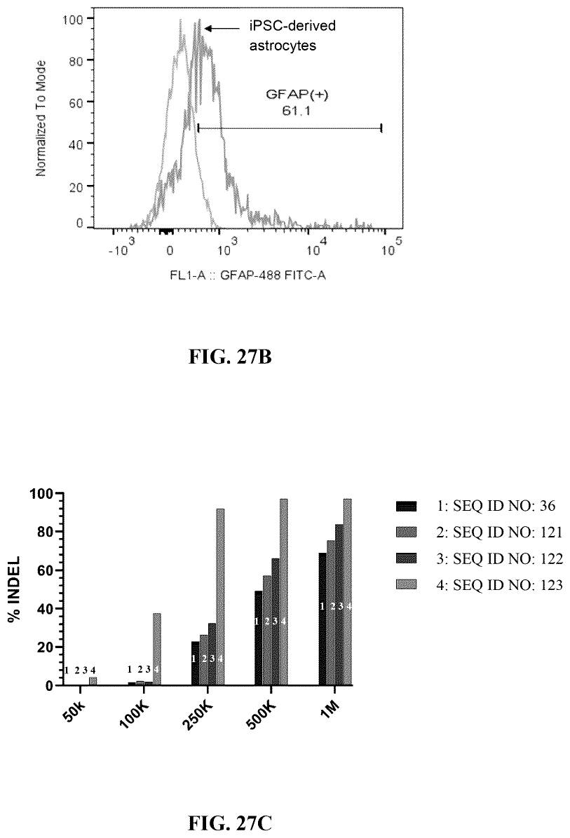

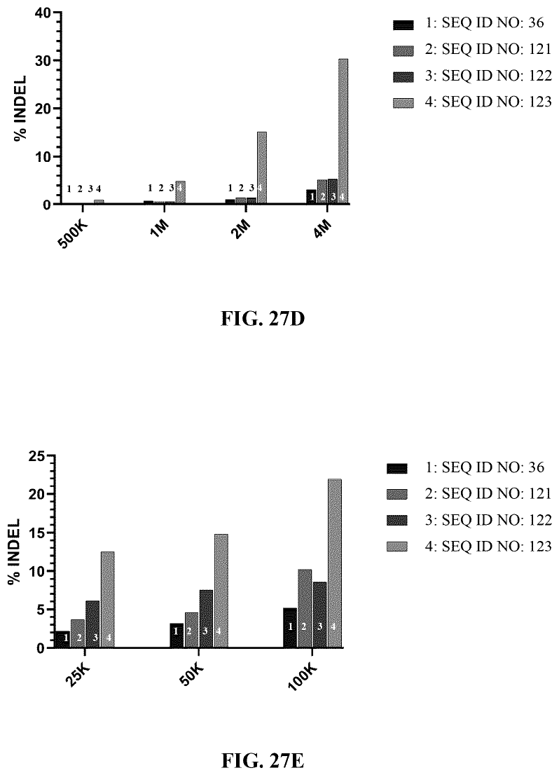

The objective of these studies was to evaluate the activity of three novel AAV cassette designs, expressing SGN004282 driven by the hU6 (249 bp) or hU6 (318 bp) promoter (set forth as SEQ ID NOs: 128 and 89, respectively) and mammalian codon optimized APG05586 (APG05586mco) directed by the CMVeb (225 bp) promoter, in editing the genomic target HTT exon50 rs362331 in either HEK293t cells or HD patient-derived fibroblasts which were reprogrammed into induced pluripotent stem cells (iPSCs) and subsequently differentiated into astrocytes. Three AAV expression cassettes have been engineered to optimize the activity of APG05586mco and SGN004282 by reducing the total AAV cassette size, utilizing different promoters to drive expression of SGN004282, different nuclear localization signals (NLS) to improve translocation of APG00586mco, or a combination thereof. In this study, all three new cassettes (and the parental construct set forth as SEQ ID NO: 36) were packaged into either an AAV5 or AAV6 viral vector and delivered at a range of multiplicities of infection (MOI). Genomic DNA was subsequently harvested from cells and subjected to NGS to quantify the rate of editing at the genomic target, HTT exon50 rs362331, as measured by INDELs. Confirmation of astrocyte differentiation by immunocytochemistry and flow cytometry are shown in FIGS. 27A and 27B, respectively. INDEL rates from in vitro models are provided in Tables 31-33 and FIGS. 27C-27E.

The results from these studies demonstrate the improved activity of the novel AAV constructs (SEQ ID NO: 121, 122, or 123) over the previous design (SEQ ID: NO 36) in generating INDELs in different types of cells and by delivery through different AAV serotypes. These findings reveal that the incorporation of c-MYC NLSs (SEQ ID NO: 125) and linker proteins (SEQ ID NO: 127) dramatically improved INDEL rates between 1.5×-30× fold depending on the cell type, serotype, and dose level tested.

### Methods

iPSC-Derived Astrocyte Differentiation

iPSCs were differentiated to astrocyte using manufacturer's manual for STEMdiff™ SMADi Neural Induction Kit, followed by STEMdiff™ Astrocyte Differentiation Kit and STEMdiff™ Astrocyte Maturation Kit (StemCell Technologies).

Characterization of iPSC Derived Astrocytes

To confirm astrocyte differentiation, iPSC-derived astrocytes were harvested for immunofluorescence and flow cytometry analysis of astrocyte markers. For immunofluorescence assay, cells were cultured on chamber slide, fixed by 4% paraformaldehyde and permeabilized with 0.1% Triton x-100 at RT for 20 mins. The cells were blocked with 56 normal goat serum and stained with anti-GFAP-488 overnight at 4° C. Nuclei were counterstained with DAPI. Images were captured by EVOS 5000 microscope, (FIG. 27A). For flow cytometry assay, iPSC-derived astrocytes were dissociated with TrypLE, then fixed by 1.6% o paraformaldehyde. Fixed cells were permeabilized and stained with anti-GFAP-488 antibody. Data was collected with CytoFlex and analyzed with FlowJo. The flow cytometry result showed that 6100 of the population is GFAP(+) astrocytes (FIG. 27B).

### AAV Transduction Conditions for 1PSC-Derived Astrocytes

iPSC-derived astrocytes were dissociated and seeded at a density of 1e5 vc/cm² in STEMdiff™ Astrocyte Maturation Media. 24 hrs after replating, AAV particles were added onto astrocyte culture at MOI of 25K, 50K and 100K, respectively. After 16 hrs of incubation, cells were replenished with fresh Astrocyte Maturation Media. Media was changed every other day.

INDEL Analysis of iPSC-Derived Astrocytes

Cell samples of astrocytes (ACs) are collected 7 days post AAV transduction. Genomic DNA was isolated by QuickExtract reagent using ACell (Highres Biosolutions), PCR was performed by Inheco ODTC, and PCR cleanups were done by an Agilent Bravo. PCR products were submitted for NGS sequencing.

TABLE 31
INDEL Rates Following AAV6 Transduction in HEK293t Cells

| | SEQ ID NO: | SEQ ID NO: | SEQ ID NO: | SEQ ID NO: |
|---|---|---|---|---|
| MOI | 36 | 121 | 122 | 123 |
| 50,000 | 0.13 | 0.28 | 0.37 | 4.24 |
| 100,000 | 1.77 | 2.45 | 2.06 | 37.71 |
| 250,000 | 22.8 | 26.38 | 32.51 | 92 |
| 500,000 | 49.23 | 57.14 | 66.19 | 97.22 |
| 1,000,000 | 68.96 | 75.49 | 83.79 | 97.18 |

TABLE 32
INDEL Rates Following AAV5 Transduction in HEK293t Cells

| | SEQ ID NO: | SEQ ID NO: | SEQ ID NO: | SEQ ID NO: |
|---|---|---|---|---|
| MOI | 36 | 121 | 122 | 123 |
| 500,000 | 0 | 0.24 | 0.22 | 1.04 |
| 1,000,000 | 0.87 | 0.69 | 0.73 | 4.93 |
| 2,000,000 | 1.18 | 1.51 | 1.56 | 15.17 |
| 4,000,000 | 3.15 | 5.18 | 5.34 | 30.31 |

TABLE 33
INDEL Rates Following AAV6 Transduction
in iPSC-Derived Astrocytes

| | SEQ ID NO: | SEQ ID NO: | SEQ ID NO: | SEQ ID NO: |
|---|---|---|---|---|
| MOI | 36 | 121 | 122 | 123 |
| 25,000 | 2.2 | 3.7 | 6.1 | 12.5 |
| 50,000 | 3.2 | 4.6 | 7.5 | 14.8 |
| 100,000 | 5.2 | 10.2 | 8.6 | 21.9 |

### Example 13: NHP Dose Sighting Study: Biodistribution and RNA Expression

The objective of this study was to evaluate the one-month tolerability and biodistribution of the Test Article (AAV5-packaged pAAV-hU6(249 bp)-SGN004282-CMVeb-c-MYC-NLS-APG05586mco-c-MYC-NLS-SV40pA(179 bp) (SEQ ID NO: 123)) following bilateral intrastriatal administration in adult cynomolgus monkeys.

Six cynomolgus monkeys were dosed into the caudate and putamen (bilateral) using the Renishaw Neuroinfuse™-intraparenchymal acute drug delivery system and convection-enhanced delivery (CED) with real-time MRI visualization. In-life observations and measurements included clinical observations, body weight, clinical pathology evaluations, and follow-up MRI. Samples were collected for biodistribution and immunology assessment (ELISpot). On Study Day 29±3, the animals were euthanized, and selected tissues were harvested for safety and biodistribution, and transgene expression evaluation.

The Test Article doses were chosen based on multiples of activity exposure from the BACHD murine studies. The low dose, 1.59E12 vg/NHP, was determined by the pharmacological active dose, which is defined by the dose required to generate a 40% reduction in mutant huntingtin protein. Previous studies showed a 43.2% reduction in mutHTT to wtHTT protein ratio was generated in the mouse striatum 12-weeks following intrastriatal administration of 2.05E10 vg AAV5.SGN4282.CMV.APG05586mco/animal.

Utilizing allometric striatal volume scaling, BACHD mice (25.0 mm³) and cynomolgus monkeys (1940 mm³), the 2.05E10 vg dose for BACHD mice equates to a 1.59E12 vg dose for cynomolgus monkeys. The administration placement and volumes (75 μL caudate and 150 μL putamen per hemisphere) were chosen to achieve maximal exposure in the striatum. See Table 34.

TABLE 34
NHP Administration Schedule

| Group | N | Titer (vg/mL) | Dose (vg/brain) |
|---|---|---|---|
| vehicle | 1 | 0 | 0 |
| Low dose | 2 | 5.0E12 | 2.2E12 |
| High dose | 3 | 2.5E13 | 1.1E13 |

qPCR was used to detect the AAV vector genome to assess biodistribution and RNA expression. The expression of APG05586mco was assayed by Jess Western CE, as described in Example 9.

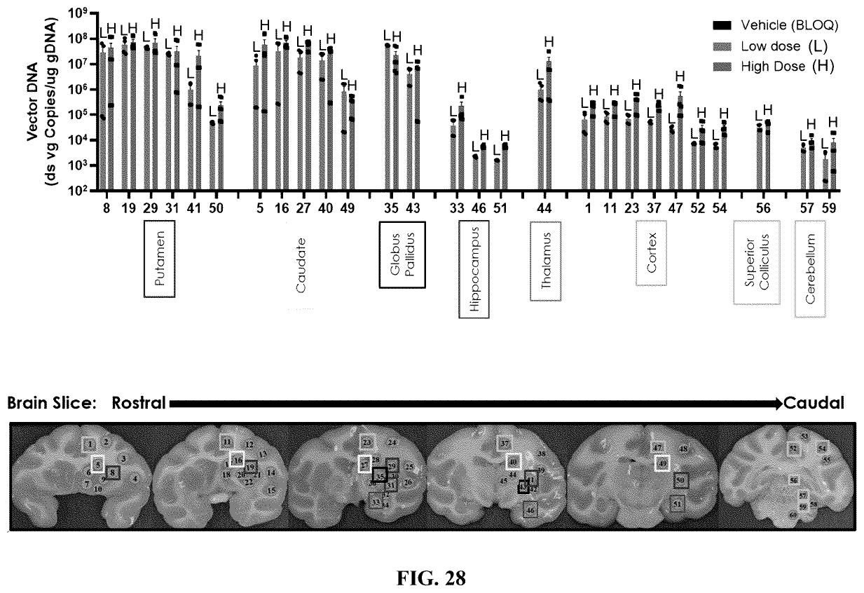

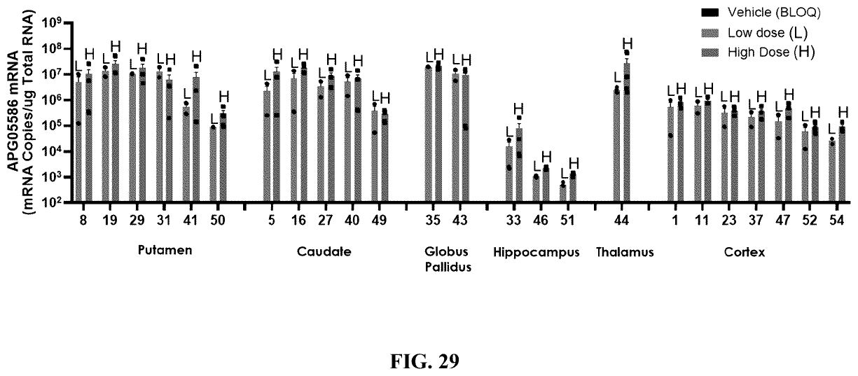

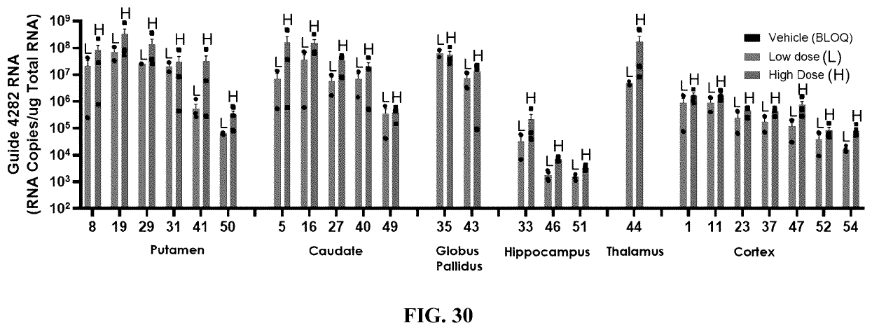

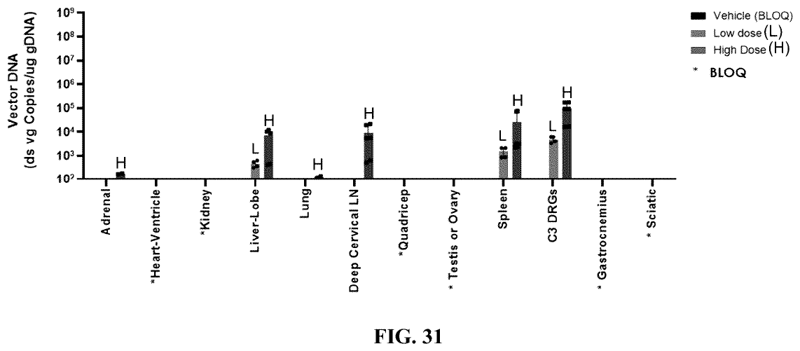

FIG. 28 shows the biodistribution of the Test Article following bilateral intrastriatal administration in adult cynomolgus monkey. FIG. 29 shows the expression of APG05586mco and FIG. 30 shows the expression of SGN004282 guide RNA in the brain following bilateral intrastriatal administration in adult cynomolgus monkey. FIG. 31 shows the biodistribution of the Test Article in peripheral tissue following bilateral intrastriatal administration in adult cynomolgus monkey.

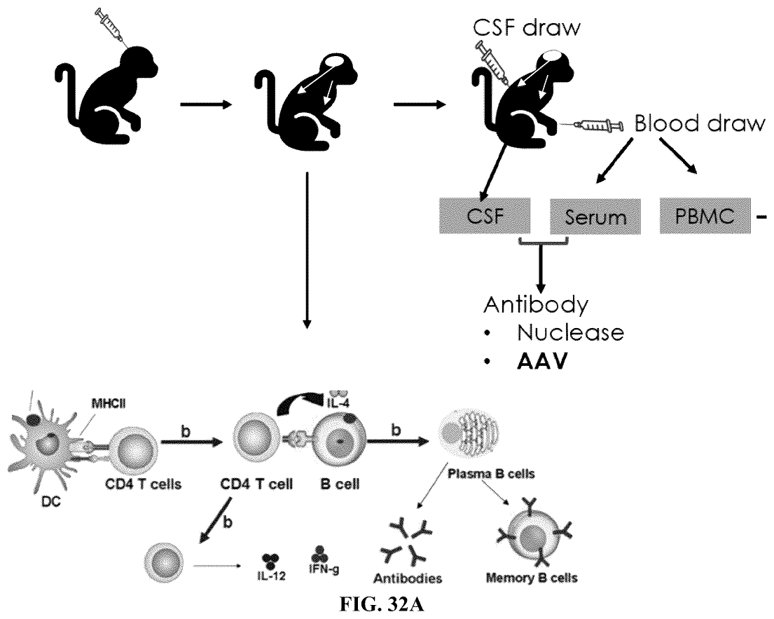

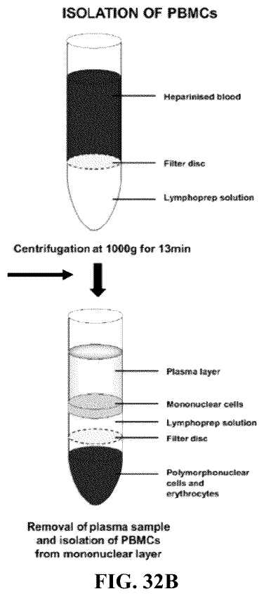

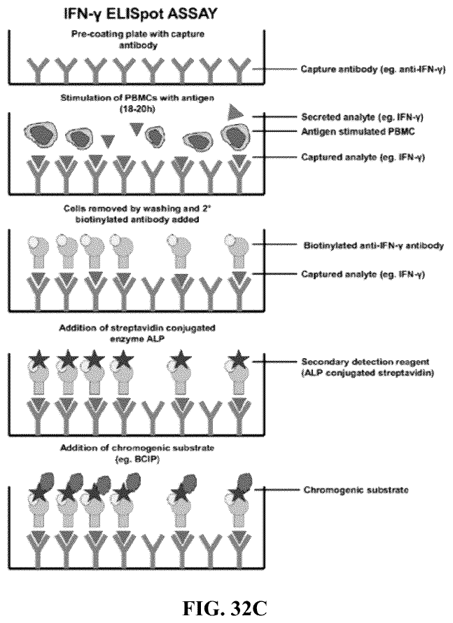

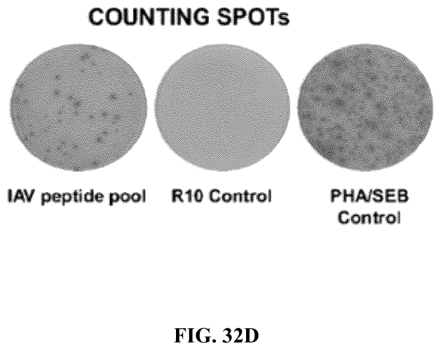

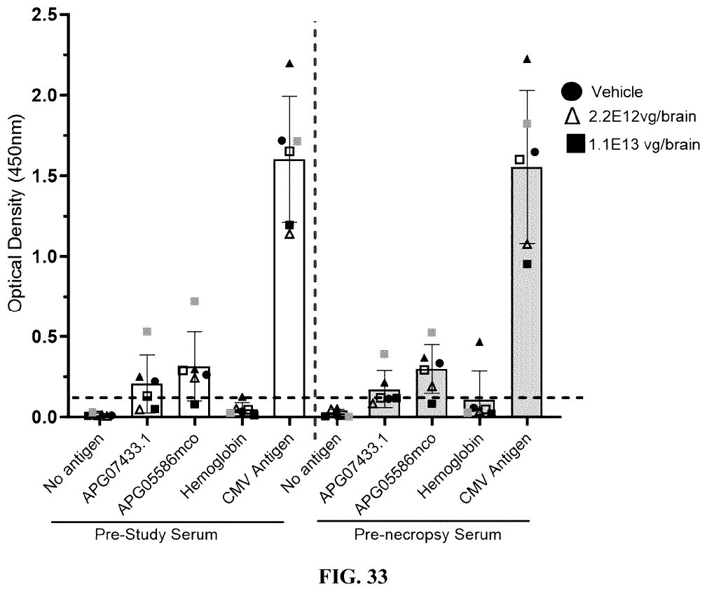

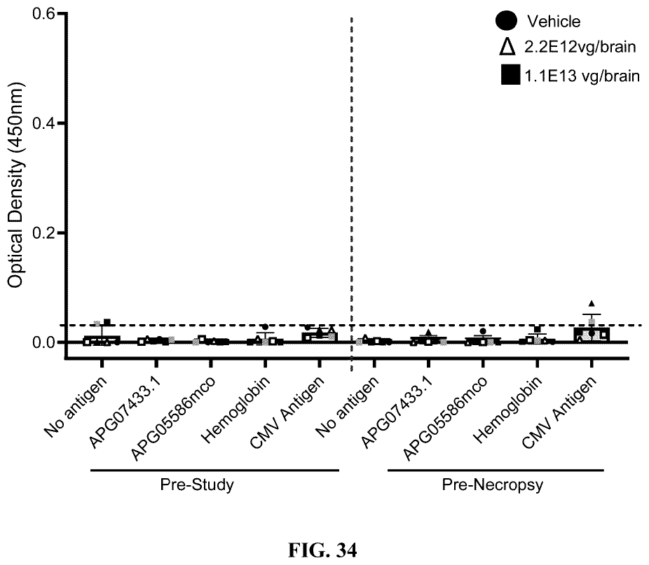

FIGS. 32A-32C show a scheme for immunogenicity testing that was performed on samples obtained from the non-human primates before and after administration of the Test Article. As shown in FIG. 33, no increase in serum antibodies reactive to APG05586mco was observed after administration in the non-human primates. FIG. 34 shows negligible levels of anti-APG05586mco antibodies were observed in the CSF of cynomolgus monkeys before and after administration of the Test Article.

Detailed clinical observations, including body weight and food consumption monitoring, ophthalmic examination, and functional observational battery revealed no untoward outcomes. Bilateral intrastriatal administration of the Test Article resulted in high vector copy number, nuclease and guide expression across brain regions that are critically vulnerable in Huntington's Disease. Minimal AAV vector was observed systemically, with nothing detected in the gonads. No change in immune response to the nuclease was observed.

### Example 14: Dose-Dependent AAV Distribution, Nuclease Transgene Expression, and mutHTT Protein Reduction in a Clinically Relevant Huntington's Disease Murine Model

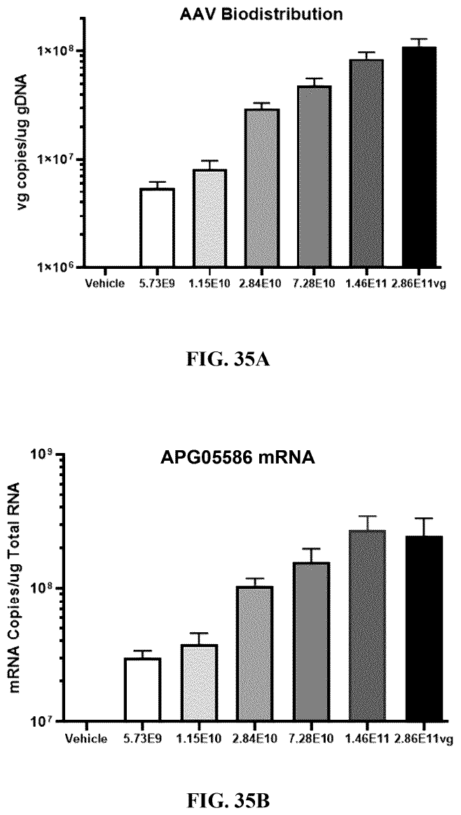

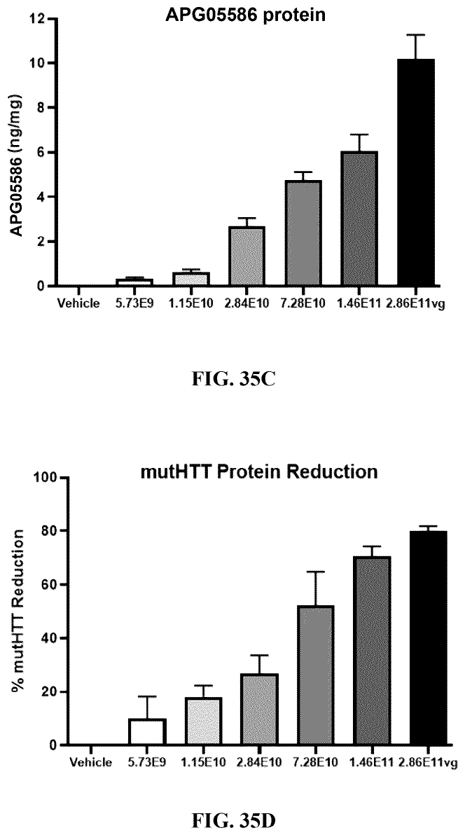

Four weeks following intrastriatal administration of vehicle or AAV5-packaged pAAV-hU6(249 bp)-SGN004282-CMVeb-c-MYC-NLS-APG05586mco-c-MYC-NLS-SV40pA(179 bp) (SEQ ID NO: 123) to BACHD mice, striatal tissues were harvested, and bulk lysate tissue samples assess for AAV vector, nuclease transgene expression (mRNA and protein) and muHTT protein reduction. Each point represents mean±SE with 4 to 6 animals per dose evaluated. Vehicle treated animals were below the LLOQ for the vector and transgene assays with percent muHTT protein reduction being zero. Test article animals were administered 8 μl/brain across 4 injection sites. Results are shown in FIGS. 35A-35D. These data show a dose-dependent AAV copy number, transgene expression, and clinically relevant (˜40%) reduction in mutHTT protein.

TABLE 35
Sequences of the application.

| SEQ | | |
|---|---|---|
| ID | | |
| NO | Description | Sequence |
| 1 | HTT Exon 50 “T” | GCCCTGTCCTGGCATTTGATCCATGAGCAGATCCCGCTGAGTCTGGATCTCCAGGCAGGGCTGGACT |
| | allele | GCTGCTGCCTGGCCCTGCAGCTGCCTGGCCTCTGGAGCGTGGTCTCCTCCACAGAGTTTGTGACCCAC |
| | | GCCTGCTCCCTCATCTACTGTGTGCACTTCATCCTGGAGGCCG |
| 2 | HTT Exon 50 “C” | GCCCTGTCCTGGCATTTGATCCATGAGCAGATCCCGCTGAGTCTGGATCTCCAGGCAGGGCTGGACT |
| | allele | GCTGCTGCCTGGCCCTGCAGCTGCCTGGCCTCTGGAGCGTGGTCTCCTCCACAGAGTTTGTGACCCAC |
| | | GCCTGCTCCCTCATCCACTGTGTGCACTTCATCCTGGAGGCCG |
| 3 | APG07433.1 | MRELDYRIGLDIGTNSIGWGVIELSWNKDRERYEKVRIVDQGVRMFDRAEMPKTGASLAEPRRIARSSRR |
| | nuclease | RLNRKSQRKKNIRNLLVQHGVITQEELDSLYPLSKKSMDIWGIRLDGLDRLLNHFEWARLLIHLAQRRGF |
| | | KSNRKSELKDTETGKVLSSIQLNEKRLSLYRTVGEMWMKDPDFSKYDRKRNSPNEYVFSVSRAELEKEIV |
| | | TLFAAQRRFQSPYASKDLQETYLQIWTHQLPFASGNAILNKVGYCSLLKGKERRIPKATYTFQYFSALDQ |
| | | VNRTRLGPDFQPFTKEQREIILNNMFQRTDYYKKKTIPEVTYYDIRKWLELDETIQFKGLNYDPNEELKKI |
| | | EKKPFINLKAFYEINKVVANYSERTNETFSTLDYDGIGYALTVYKTDKDIRSYLKSSHNLPKRCYDDQLIE |
| | | ELLSLSYTKFGHLSLKAINHVLSIMQKGNTYKEAVDQLGYDTSGLKKEKRSKFLPPISDEITNPIVKRALTQ |
| | | ARKVVNAIIRRHGSPHSVHIELARELSKNHDERTKIVSAQDENYKKNKGAISILSEHGILNPTGYDIVRYKL |
| | | WKEQGERCAYSLKEIPADTFFNELKKERNGAPILEVDHILPYSQSFIDSYHNKVLVYSDENRKKGNRIPYT |
| | | YFLETNKDWEAFERYVRSNKFFSKKKREYLLKRAYLPRESELIKERHLNDTRYASTFLKNFIEQNLQFKE |
| | | AEDNPRKRRVQTVNGVITAHFRKRWGLEKDRQETYLHHAMDAIIVACTDHHMVTRVTEYYQIKESNKS |
| | | VKKPYFPMPWEGFRDELLSHLASQPIAKKISEELKAGYQSLDYIFVSRMPKRSITGAAHKQTIMRKGGIDK |
| | | KGKTIIIERLHLKDIKFDENGDFKMVGKEQDMATYEAIKQRYLEHGKNSKKAFETPLYKPSKKGTGNLIK |
| | | RVKVEGQAKSFVREVNGGVAQNGDLVRVDLFEKDDKYYMVPIYVPDTVCSELPKKVVASSKGYEQWL |
| | | TLDNSFTFKFSLYPYDLVRLVKGDEDRFLYFGTLDIDSDRLNFKDVNKPSKKNEYRYSLKTIEDLEKYEV |
| | | GVLGDLRLVRKETRRNFH |
| 4 | APG07433.1 crRNA | GUCAUAGUUCCAUUAAAGCCA |
| | repeat | |
| 5 | APG07433.1 | UGGCUUUGAUGUUUCUAUGAUAAGGGUUUCGACCCGUGGCGUCGGGGAUCGCCUGCCCAUUGAA |
| | tracrRNA | AUGGGCUUCUCCCCAUUUAUU |
| 6 | APG07433.1 generic | NNNNNNNNNNNNNNNNNNNNNNNNNGUCAUAGUUCCAUUAAAGCCAAAAGUGGCUUUGAUGUUU |
| | sgRNA | CUAUGAUAAGGGUUUCGACCCGUGGCGUCGGGGAUCGCCUGCCCAUUGAAAUGGGCUUCUCCCCA |
| | | UUUAUU |
| 7 | APG05586 nuclease | MYSIGLDLGISSVGWSVIDEETGKIVDLGVRLFSAKNSEKNLERRTSRGARRLIRRKTNRLKDAKKLLEAI |
| | | GFYEDKALKNVCPYQLRVKGLTEGLTKGELYKVVLHIVKKRGISYLDEDDAEAAKESQDYKEQVRKNA |
| | | QLLTKYTPGQIQLQRLKENNRVKTGINGQGHYQLNVFKVSAYADELATILKTQQALYPNELTDDWIALF |
| | | VQPGIAENAGLIYRKRPYYHGPGNEANNSPYGRWSDFQKTGQPAANIFDKLIGKDFQGELRASGLSLSAQ |
| | | QYNLLNDLTNLKIDGEVSLSPEQKEFILTELMTKEFARFGVNDIAKLLGVKKEQLSGWRLDKKGKPEIHT |
| | | LKGYRNWRKIFAEAGIDLATLPTETIDCLAKVLTLNTEREGVENTLAFELPELAEPVKSLVLDHYKELSQS |
| | | ISTQAWHRFSLKTLHLLIPELIKSTSEQNTLLEQFQLKAGVRKRYSDYKKLPTKEVLAEIYNPTVNKTVSQ |
| | | AFKVMDALLEKYGKDQIHYITVEMPRDDNEEEERKRIKELQTKNSQRKNDSQQYFLQKSGWSQEKFQAT |
| | | IHKNRRFLAKLLYYFEQDGVCAYTGNPISPELLVSDSTEIDHIIPISISLDDSINNKVLVLSHANQVKGQQTP |
| | | YDARMAGAFNKINGKFSNWDEYQKWVESRPFSRKKVNNLLETRNIFDSEQVQKFLSRNLNDTRYASRLV |
| | | LNTLQSFFENQDTIVRVVNGSFTHTLRKKWGADLDKTRETHHHHAVDATLCAVTPFVKVSRYHYAVNE |
| | | ETGEKFMREIDVETGEILDEIPYREYKKAKHYERKTYQVKWSNFREQLKPITIHPKIKFSHQVDRKANRKL |
| | | SDATIYSVREKTEVKTLKSGKEKITTDEYTIGKIKDIYTVDGWEAFKKKQDKLLMKEFDEKTYELLVTIAA |
| | | TTPDFQEVEEKNGKVKRVKRSPFAVYCEENGIPAIRKYAKKNNGPVIRSLKYYDGKLNKHINITKDEKGR |
| | | PVEQTKNGRKVTLQSLKPYRYDIYQDLETKAYYTVQLYYSDLRFVEGEYGITEKEYMKKVAEQTKGQV |
| | | VRFCFSLQKNDGLEIEWKDSQRYDVRFYNFQSANSINFKGLEQEMIPAENQFKQKPYNNGAINLNIAKYG |
| | | KEGKKLRKFNTDILGKKHHLSYEKEPKNIIK |
| 8 | APG05586 crRNA | GUUAUUGUACUCUCAAUAA |
| | repeat | |
| 9 | APG05586 tracrRNA | UUAUUGAGAAUCUACAAUAAUAAGGCAUCUUGCCGAAUUUACCGCCCUACAUAUGUAGGGCGGU |
| | | UUU |
| 10 | APG05586 generic | NNNNNNNNNNNNNNNNNNNNNNNNNNNNNGUUAUUGUACUCUCAAUAAAAAGUUAUUGAGAAUC |
| | sgRNA | UACAAUAAUAAGGCAUCUUGCCGAAUUUACCGCCCUACAUAUGUAGGGCGGUUUU |
| 11 | APG01604 nuclease | MVTKYILGLDIGITSVGYGIINYEDKTIIDAGVRLFPEANVENNEGRRSKRGARRLKRRRIHRLDRIKQLLS |
| | | EYNLVDLDNIPQSPSPYEIRVKGLREELSKDELVIALLHIAKRRGIHNVEAVDETQDEGNELSTKEQLAKN |
| | | NNLLKDKYVCELLLERLKDGKVRGEKNRFKTTDIIKEVKQLLETQKEAHQLDDDFINRYIDLIETRREYFE |
| | | GPGKGSPFGWGGDLKKWYETLMGHCTYFPNELRSVKYSYSADLFNALNDLNNLVIQREGNSKLEYHEK |
| | | YHIIENVFKQKKKPTLKQIANEIGVSPDDIKGFRINKSGKETFTEFKLYHDLKKVLIDQSILENVQLLDQIAE |
| | | ILTIYQDKESIINELNQLSEIINEQDKESISNLSGYNGTHRLSLKCINLVIEELWHTSRNQMEIFTYLNIKPKKI |
| | | DLAKTNKIPKNMIDEFILSPVVKRTFGQAINVINKVIEKYGVPEDIIIELARESNSKDKQKFINSLQKKNETT |
| | | RKRINEIIGQYGNQNAKRLVEKIRLHDEQEGKCLYSLESIPLEDLINNPQYYEVDHIIPRSVSFDNSYQNKV |
| | | LVKQTENSKKSNRTPYQYFNSGETKLSYNQFKQHVLNLSKSKDRISKKKKEYLLEERDINKYEVQKEFIN |
| | | RNLVDTRYATRELTNYLKAYFSANDMDVKVKTINGSFTDYLRKVWKFKKERNHGYKHHAEDALIIANA |
| | | DFLFKENKKLKKANAILEQPSLDNGKSDATVENDNEYVETFSIPKQVNDIKEFRDFKFSHRVDKKPNRQLI |
| | | NDTLYSTRKIENHTFIVSPITNIYSKDNDELKKKFNKNPEKFLMYQHDPKTFEKLEVIMKQYANEKNPLAK |
| | | YHEETGEYLTKYSKKNNGPIVKTIKVLGDKVGKHLDVTHKYKYSNSKIVKKTINPYRFDVYLTDKGYKFI |
| | | TISYLDVLKKDNYYYILKEKYEELKIKKSISDTDQFIGSFYYNDLIKINDQIFKVVGVNNDLLNRIELDLLDI |
| | | SYKEYCKINNIKTNRIIKGITKKITNIEKFSTDVLGNLYKAHSNHPQLIFKQRD |
| 12 | APG01604 crRNA | GUUUUAGUACUCUGUAA |
| | repeat | |
| 13 | APG01604 tracrRNA | UUACAGAAUCUACUAAAACAAGACUAUAUAUCGUGUUUAUCCCAUCAAUUUAUUGAUGGGAUUU |
| 14 | APG01604 generic | NNNNNNNNNNNNNNNNNNNNNNNNNNNNNGUUUUAGUACUCUGUAAAAAGUUACAGAAUCUACU |
| | sgRNA | AAAACAAGACUAUAUAUCGUGUUUAUCCCAUCAAUUUAUUGAUGGGAUUU |
| 15 | LPG10145 nuclease | MSKVAKNMTRVNLGFDIGIASVGWSVLDNQTGKILETGVSIFPSGSASRNEERRSFRQSRRLIRRKKARIC |
| | | DMDHLLKKNGFPFPGNTGANPYEIRVKGLTEKLSREELAIALHHLVKRRGISYDLKDVEDESAGGTNYQE |
| | | SIAVNQRLLKKETPAEIQLARLTECGKVRGQVKSLGEDNTATTLLNVFPNAAYQEEMVKLLKKQQEFYG |
| | | EIDDPFMETAIGILSRKREYFIGPGSEKSRTDYGIYRTDGTTLKNLFEILVGKDKIFPDQYRAAGNSYTAQL |
| | | YNLLNDLNNLEVDATEDGKLTTAHKEQIIEELTTTTGNVNMLKLIAKVAGTSPAGIKKYRVDREGKPEFH |
| | | SLAIYRRLRKKLGEAGFEINEWPPEFFDDYGPIVTLNTESGELRKWLAEEGSRKYDFLTEPVIEAILANKSA |
| | | FDSVGKNKWHRFSLKTMQLLIPELLHTFKEQMTILAEMGLLHENKKDYGDQNKVDVKYLTENLYNPVV |
| | | RKSVKQAMDIFNALFEKYANIDYVVIEMPRDDAEDELEQKKQFQKFQLKNEKEKDASLKEFQELAGVSD |
| | | LQLEAQLRKRKKLRQKIRMWYQQRGKCPYSGKTIAAVDLFHQDNQFEIDHIIPLSVSFDDGQNNKVLCYS |
| | | EMNQEKGKQTPYAFMSRGGGQGFSALQAYVKSNNRLENAKKRNLLFTEDINDLEVRKRFIARNLVDTR |
| | | YASRIVLNELQQFVRSKELDTRVTVIRGKLTSKLRDRWRLNKSRETHHHHAVDAAVIAVSPMLKMWEK |
| | | NAEIIPLKVDENTVDLKSGEIITDQEYAAQMYELPYARFLEQMPELHKKIKFHHQVDKKMNRKVSDATLY |
| | | STRKAKVGTDKKEQEYVIGKIKDIYQFDQYKKFKKLYDGDKSKFLMQRLDPQTFAKLEKIMEDYPAKID |
| | | ATQPNGTIKLVDISPFELYRREHGPVTKYAKKNNGPAIKSLKFYDSIVGSSVKITPKNAKGKEVILKSLKP |
| | | WRTDVYYNHEKEQYEIMGIKYADLKFKGDNYGITKARYQEIKEEEGVSEESEFLFSLYRGDRIQVSNGED |
| | | KIDLLFLSRSNPAKKGYVELKPIDRNQLNGKEVVSVYGAASGGRLKKQFVKKNHTLHKVNTDILGNPFYI |
| | | KKESDQPKNILDL |
| 16 | LPG10145 crRNA | GUUAUUGUACUCUCAAGGAAUCAG |
| | repeat | |
| 17 | LPG10145 tracrRNA | CUGAUUCCUUGAGAAUCUACAAUAAUAAGGCUUUAUGCCGAAAUUCCCACUCUUAACGGAGUGG |
| | | GUUUU |
| 18 | LPG10145 generic | NNNNNNNNNNNNNNNNNNNNNNNGUUAUUGUACUCUCAAGGAAUCAGaaagCUGAUUCCUUGAGA |
| | sgRNA | AUCUACAAUAAUAAGGCUUUAUGCCGAAAUUCCCACUCUUAACGGAGUGGGUUUU |
| 19 | rs362331 forward | AAACGAAGGTACACGAGTGG |
| | primer | |
| 20 | rs362331 reverse | GCAAACAGGCAGCACAAAAT |
| | primer | |
| 21 | HTT forward exon 1 | AGCTGATGGGCGCCTTCGAGTCCCTCAAGTC |
| | primer | |
| 22 | HTT reverse exon 50 | TGCACTGCAGGCGCCTCCAGGATGAAGTGCACAC |
| | primer | |
| 23 | HTT inverse forward | CAGATCCCGCTGAGTCTGGATCTCC |
| | exon 50 primer | |
| 24 | HTT inverse reverse | CGGCTGAGGCAGCAGCGGCT |
| | exon 1 primer | |
| 25 | SGN004281 | ACCGGCCUCCAGGAUGAAGUGCACAGUUAUUGUACUCUCAAUAAAAAGUUAUUGAGAAUCUACA |
| | | AUAAUAAGGCAUCUUGCCGAAUUUACCGCCCUACAUAUGUAGGGCGGUUUU |
| 26 | SGN004282 | ACCGGCCUCCAGGAUGAAGUGCACAGUUAUUGUACUCAAAGGAAUCUACAAUAAUAAGGCAUCU |
| | | UGCCGAAUUUACCGCCCUACAUAUGUAGGGCGGUUUU |
| 27 | SGN002908 | GAGUUUGUGACCCACGCCUGCUCCCGUCAUAGUUCCAUUAAAGCCAAAAGUGGCUUUGAUGUUUC |
| | | UAUGAUAAGGGUUUCGACCCGUGGCGUCGGGGAUCGCCUGCCCAUUGAAAUGGGCUUCUCCCCAU |
| | | UUAUU |
| 28 | SGN002911 | UUUGUGACCCACGCCUGCUCCCGUCAUAGUUCCAUUAAAGCCAAAAGUGGCUUUGAUGUUUCUAU |
| | | GAUAAGGGUUUCGACCCGUGGCGUCGGGGAUCGCCUGCCCAUUGAAAUGGGCUUCUCCCCAUUUA |
| | | UU |
| 29 | SGN004333 | CGGCCUCCAGGAUGAAGUGCACACAGUUUUAGUACUCUGUAAaaagUUACAGAAUCUACUAAAACA |
| | | AGGCAAAAUGCCGUGUUUAUCUCGUCAACUUGUUGGCGAGAUUU |
| 30 | pAAV-hU6(318 bp)- | GTGCACGATATCGGGTCCCCAATTGCTGCGCGCTCGCTCGCTCACTGAGGCCGCCCGGGCAAAGCCC |
| | SGN002908-JeT- | GGGCGTCGGGCGACCTTTGGTCGCCCGGCCTCAGTGAGCGAGCGAGCGCGCAGAGAGGGAGTGGCC |
| | APG07433.1- | AACTCCATCACTAGGGGTTCCTCGTCGACAATGCACGCGTTCGAAGTACTCATGTACAAAAAAGCAG |
| | SV40pA(179 bp) | GCTTTAAAGGAACCAATTCAGTCGACTGGATCCGGTACCAAGGTCGGGCAGGAAGAGGGCCTATTTC |
| | | CCATGATTCCTTCATATTTGCATATACGATACAAGGCTGTTAGAGAGATAATTAGAATTAATTTGACT |
| | | GTAAACACAAAGATATTAGTACAAAATACGTGACGTAGAAAGTAATAATTTCTTGGGTAGTTTGCAG |
| | | TTTTAAAATTATGTTTTAAAATGGACTATCATATGCTTACCGTAACTTGAAAGTATTTCGATTTCTTGG |
| | | CTTTATATATCTTGTGGAAAGGACGAAACACCGAGTTTGTGACCCACGCCTGCTCCCGTCATAGTTCC |
| | | ATTAAAGCCAAAAGTGGCTTTGATGTTTCTATGATAAGGGTTTCGACCCGTGGCGTCGGGGATCGCC |
| | | TGCCCATTGAAATGGGCTTCTCCCCATTTATTTTTTTGAGCTCGAATTCGGGCGGAGTTAGGGCGGAG |
| | | CCAATCAGCGTGCGCCGTTCCGAAAGTTGCCTTTTATGGCTGGGCGGAGAATGGGCGGTGAACGCCG |
| | | ATGATTATATAAGGACGCGCCGGGTGTGGCACAGCTAGTTCCGTCGCAGCCGGGATTTGGGTCGCGG |
| | | TTCTTGTTTGTGGATCCCTGTGATCGTCACTTGACAGCGGCCGCGCCACCATGGCCCCTAAGAAGAA |
| | | AAGAAAGGTGATGAGAGAGCTGGACTACAGAATTGGCCTGGACATCGGCACCAACAGCATCGGATG |
| | | GGGCGTGATCGAGCTGTCCTGGAACAAAGACCGGGAGAGATACGAGAAGGTCAGAATCGTGGATCA |
| | | AGGCGTGAGAATGTTCGACAGAGCCGAGATGCCCAAGACAGGCGCCAGCTTAGCTGAACCCAGAAG |
| | | AATCGCCAGATCCAGCAGACGGAGACTGAATCGCAAGTCCCAGAGAAAGAAAAACATCCGGAACCT |
| | | GCTGGTGCAACACGGCGTGATCACACAGGAGGAACTGGATAGCCTGTACCCCCTGAGCAAAAAGAG |
| | | CATGGACATCTGGGGCATTCGGCTCGACGGCCTGGACAGACTCCTCAATCATTTCGAGTGGGCCAGA |
| | | CTGCTGATCCACCTGGCTCAGAGACGGGGCTTTAAGTCCAACAGAAAGAGTGAACTGAAAGATACA |
| | | GAGACAGGCAAGGTGCTGAGCAGCATCCAACTGAACGAGAAACGGCTGAGCTTGTATAGAACCGTG |
| | | GGCGAGATGTGGATGAAGGACCCCGACTTCTCTAAATACGATAGGAAGAGAAATAGCCCCAACGAA |
| | | TACGTGTTCAGCGTGTCTAGAGCCGAGCTGGAAAAGGAAATCGTGACCCTGTTCGCCGCCCAGCGGA |
| | | GATTCCAGAGCCCTTACGCCAGCAAAGATCTGCAGGAGACATATCTGCAGATCTGGACCCACCAACT |
| | | GCCTTTCGCCAGCGGCAATGCCATCCTGAACAAGGTCGGATACTGCTCCCTGTTGAAAGGCAAAGAA |
| | | AGAAGGATTCCCAAGGCTACATACACCTTCCAATACTTCTCTGCTCTGGACCAGGTGAATCGGACCA |
| | | GACTGGGACCTGATTTCCAGCCCTTCACCAAGGAGCAACGGGAAATTATCTTGAACAACATGTTCCA |
| | | GAGGACAGATTACTACAAGAAGAAAACCATCCCCGAGGTGACCTACTATGACATACGGAAGTGGCT |
| | | GGAATTGGACGAGACAATTCAGTTCAAGGGCCTGAACTACGACCCTAACGAGGAACTGAAGAAGAT |
| | | CGAGAAGAAGCCTTTTATCAATCTGAAGGCCTTCTACGAGATCAACAAGGTGGTGGCCAACTACAGC |
| | | GAAAGAACCAACGAGACCTTCTCCACCCTGGACTACGACGGCATCGGCTACGCCCTGACCGTGTACA |
| | | AAACCGACAAGGATATCCGCAGCTACCTGAAGAGCAGTCACAACCTACCTAAGAGATGCTACGACG |
| | | ACCAACTGATCGAGGAACTGCTGAGCCTGAGCTACACAAAGTTCGGCCACCTGTCCCTGAAAGCCAT |
| | | CAACCACGTGCTGTCTATCATGCAGAAGGGCAATACCTACAAGGAAGCCGTGGACCAACTGGGCTA |
| | | CGACACCAGCGGCCTTAAGAAGGAGAAGAGGTCCAAGTTCCTGCCACCTATTTCTGATGAAATCACG |
| | | AATCCAATCGTGAAAAGGGCCCTGACCCAGGCCAGAAAAGTGGTGAACGCCATAATTAGAAGACAC |
| | | GGATCTCCTCACTCCGTGCACATCGAGCTGGCCAGAGAGCTGAGCAAGAACCACGACGAGCGGACA |
| | | AAGATCGTCAGCGCCCAGGATGAAAACTACAAGAAAAACAAGGGCGCTATCAGCATCCTGTCTGAG |
| | | CACGGCATCCTGAACCCTACAGGCTACGACATCGTGAGATACAAACTGTGGAAGGAGCAGGGCGAA |
| | | CGGTGCGCCTACAGCCTGAAGGAAATCCCTGCCGATACATTTTTCAACGAGCTGAAGAAGGAACGCA |
| | | ACGGCGCCCCTATCCTTGAAGTGGACCACATCCTGCCCTACAGCCAGTCCTTCATCGACTCCTACCAC |
| | | AACAAGGTCCTGGTGTACAGCGACGAAAACCGGAAAAAGGGCAACAGAATCCCTTATACCTACTTC |
| | | CTGGAAACCAACAAGGATTGGGAGGCCTTTGAGCGGTACGTGCGGAGCAACAAATTCTTCTCCAAG |
| | | AAAAAGCGAGAGTACCTTCTGAAGCGGGCTTATCTGCCTAGAGAATCTGAGCTGATCAAAGAACGC |
| | | CACCTGAACGACACCAGATACGCCTCTACCTTCCTGAAGAACTTCATCGAGCAGAACCTGCAGTTCA |
| | | AGGAAGCCGAGGACAACCCCAGAAAAAGACGGGTGCAAACCGTGAACGGCGTTATCACCGCCCACT |
| | | TCAGAAAGCGGTGGGGCCTGGAGAAGGACCGGCAGGAGACATACCTCCATCACGCTATGGACGCCA |
| | | TCATCGTGGCTTGTACAGACCACCACATGGTCACCAGAGTGACCGAGTACTATCAGATCAAGGAAAG |
| | | CAACAAGAGCGTGAAGAAGCCCTATTTTCCTATGCCTTGGGAAGGCTTCCGGGACGAGCTGCTGAGC |
| | | CACTTGGCTTCTCAGCCTATCGCCAAGAAAATCAGCGAGGAACTGAAGGCCGGCTACCAGAGCCTGG |
| | | ACTACATCTTCGTGTCCAGAATGCCTAAGAGAAGCATTACAGGCGCTGCTCATAAGCAGACCATCAT |
| | | GCGGAAGGGAGGAATTGACAAGAAGGGCAAAACAATCATCATCGAACGGCTGCACCTGAAGGATAT |
| | | CAAGTTCGACGAGAACGGAGATTTCAAGATGGTGGGCAAGGAACAGGACATGGCCACATACGAAGC |
| | | TATTAAACAGAGATACCTGGAGCACGGCAAGAATAGCAAGAAGGCCTTCGAGACCCCTCTGTACAA |
| | | GCCCAGCAAAAAGGGCACAGGTAACCTGATCAAGCGGGTGAAGGTGGAAGGACAGGCCAAGAGCT |
| | | TTGTGAGGGAAGTGAACGGCGGAGTGGCCCAAAATGGCGATCTGGTTAGAGTTGATTTGTTTGAGAA |
| | | GGATGATAAGTACTACATGGTCCCCATCTACGTGCCAGACACCGTGTGTAGCGAGCTGCCCAAAAAG |
| | | GTGGTCGCCAGCTCTAAGGGCTATGAGCAGTGGCTGACACTGGATAACAGCTTCACCTTTAAGTTCA |
| | | GCCTGTACCCTTATGATCTGGTGCGGCTGGTCAAGGGAGATGAGGATCGGTTCCTGTACTTTGGCAC |
| | | CCTGGACATCGACAGCGACAGACTTAACTTCAAGGACGTGAACAAGCCAAGCAAGAAGAACGAGTA |
| | | CCGGTACAGCTTGAAAACCATCGAGGACTTGGAGAAGTACGAGGTGGGCGTGCTGGGCGATCTAAG |
| | | ACTGGTCCGGAAGGAAACTCGAAGAAACTTCCACAGCGGCGGAAGCAAAAGACCTGCCGCTACAAA |
| | | GAAGGCCGGCCAGGCCAAGAAAAAGAAGTAAGCTAGCTTGACTGACTGAGATACAGCGTACCTTCA |
| | | GCTCACAGACATGATAAGATACATTGATGAGTTTGGACAAACCACAACTAGAATGCAGTGAAAAAA |
| | | ATGCTTTATTTGTGAAATTTGTGATGCTATTGCTTTATTTGTAACCATTATAAGCTGCAATAAACAAG |
| | | TTAACAACAACAATTGCATTCATTTTATGTTTCAGGTTCAGGGGGAGGTGTGGGAGGTTTTTTAAAGC |
| | | AAGTAAAAGCTTGGTAGGAACCCCTAGTGATGGAGTTGGCCACTCCCTCTCTGCGCGCTCGCTCGCT |
| | | CACTGAGGCCGGGCGACCAAAGGTCGCCCGACGCCCGGGCTTTGCCCGGGCGGCCTCAGTGAGCGA |
| | | GCGAGCGCGCAGAGGCATTTAATTAAGCAAGCTGTCATAGCTGTTTCCTGTGTGAAATTGTTATCCG |
| | | CTCACAATTCCACACAACATACGAGCCGGAAGCATAAAGTGTAAAGCCTGGGGTGCCTAATGAGTG |
| | | AGCTAACTCACATTAATTGCGTTGCGCTCACTGCCCGCTTTCCAGTCGGGAAACCTGTCGTGCCAGCT |
| | | GCATTAATGAATCGGCCAACGCGCGGGGAGAGGCGGTTTGCGTATTGGGCGCTCTTCCGCTTCCTCG |
| | | CTCACTGACTCGCTGCGCTCGGTCGTTCGGCTGCGGCGAGCGGTATCAGCTCACTCAAAGGCGGTAA |
| | | TACGGTTATCCACAGAATCAGGGGATAACGCAGGAAAGAACATGTGAGCAAAAGGCCAGCAAAAG |
| | | GCCAGGAACCGTAAAAAGGCCGCGTTGCTGGCGTTTTTCCATAGGCTCCGCCCCCCTGACGAGCATC |
| | | ACAAAAATCGACGCTCAAGTCAGAGGTGGCGAAACCCGACAGGACTATAAAGATACCAGGCGTTTC |
| | | CCCCTGGAAGCTCCCTCGTGCGCTCTCCTGTTCCGACCCTGCCGCTTACCGGATACCTGTCCGCCTTT |
| | | CTCCCTTCGGGAAGCGTGGCGCTTTCTCATAGCTCACGCTGTAGGTATCTCAGTTCGGTGTAGGTCGT |
| | | TCGCTCCAAGCTGGGCTGTGTGCACGAACCCCCCGTTCAGCCCGACCGCTGCGCCTTATCCGGTAACT |
| | | ATCGTCTTGAGTCCAACCCGGTAAGACACGACTTATCGCCACTGGCAGCAGCCACTGGTAACAGGAT |
| | | TAGCAGAGCGAGGTATGTAGGCGGTGCTACAGAGTTCTTGAAGTGGTGGCCTAACTACGGCTACACT |
| | | AGAAGAACAGTATTTGGTATCTGCGCTCTGCTGAAGCCAGTTACCTTCGGAAAAAGAGTTGGTAGCT |
| | | CTTGATCCGGCAAACAAACCACCGCTGGTAGCGGTGGTTTTTTTGTTTGCAAGCAGCAGATTACGCG |
| | | CAGAAAAAAAGGATCTCAAGAAGATCCTTTGATCTTTTCTACGGGGTCTGACGCTCAGTGGAACGAA |
| | | AACTCACGTTAAGGGATTTTGGTCATGAGATTATCAAAAAGGATCTTCACCTAGATCCTTTTAAATTA |
| | | AAAATGAAGTTTTAAATCAAGCCCAATCTGAATAATGTTACAACCAATTAACCAATTCTGATTAGAA |
| | | AAACTCATCGAGCATCAAATGAAACTGCAATTTATTCATATCAGGATTATCAATACCATATTTTTGAA |
| | | AAAGCCGTTTCTGTAATGAAGGAGAAAACTCACCGAGGCAGTTCCATAGGATGGCAAGATCCTGGT |
| | | ATCGGTCTGCGATTCCGACTCGTCCAACATCAATACAACCTATTAATTTCCCCTCGTCAAAAATAAGG |
| | | TTATCAAGTGAGAAATCACCATGAGTGACGACTGAATCCGGTGAGAATGGCAAAAGTTTATGCATTT |
| | | CTTTCCAGACTTGTTCAACAGGCCAGCCATTACGCTCGTCATCAAAATCACTCGCATCAACCAAACC |
| | | GTTATTCATTCGTGATTGCGCCTGAGCGAGACGAAATACGCGATCGCTGTTAAAAGGACAATTACAA |
| | | ACAGGAATCGAATGCAACCGGCGCAGGAACACTGCCAGCGCATCAACAATATTTTCACCTGAATCA |
| | | GGATATTCTTCTAATACCTGGAATGCTGTTTTTCCGGGGATCGCAGTGGTGAGTAACCATGCATCATC |
| | | AGGAGTACGGATAAAATGCTTGATGGTCGGAAGAGGCATAAATTCCGTCAGCCAGTTTAGTCTGACC |
| | | ATCTCATCTGTAACATCATTGGCAACGCTACCTTTGCCATGTTTCAGAAACAACTCTGGCGCATCGGG |
| | | CTTCCCATACAAGCGATAGATTGTCGCACCTGATTGCCCGACATTATCGCGAGCCCATTTATACCCAT |
| | | ATAAATCAGCATCCATGTTGGAATTTAATCGCGGCCTCGACGTTTCCCGTTGAATATGGCTCATAACA |
| | | CCCCTTGTATTACTGTTTATGTAAGCAGACAGTTTTATTGTTCATGATGATATATTTTTATCTTGTGCA |
| | | ATGTAACATCAGAGATTTTGAGACACGGGCCAGAGCTGCATCGCGCGTTTCGGTGATGACGGTGAAA |
| | | ACCTCTGACACATGCAGCTCCCGGAGACGGTCACAGCTTGTCTGTAAGCGGATGCCGGGAGCAGACA |
| | | AGCCCGTCAGGGCGCGTCAGCGGGTGTTGGCGGGTGTCGGGGCTGGCTTAACTATGCGGCATCAGAG |
| | | CAGATTGTACTGAGAGTGCACGATATCGGGTCC |
| 31 | pAAV-hU6(318 bp)- | GTGCACGATATCGGGTCCCCAATTGCTGCGCGCTCGCTCGCTCACTGAGGCCGCCCGGGCAAAGCCC |
| | SGN002911-JeT- | GGGCGTCGGGCGACCTTTGGTCGCCCGGCCTCAGTGAGCGAGCGAGCGCGCAGAGAGGGAGTGGCC |
| | APG07433.1- | AACTCCATCACTAGGGGTTCCTCGTCGACAATGCACGCGTTCGAAGTACTCATGTACAAAAAAGCAG |
| | SV40pA(179 bp) | GCTTTAAAGGAACCAATTCAGTCGACTGGATCCGGTACCAAGGTCGGGCAGGAAGAGGGCCTATTTC |
| | | CCATGATTCCTTCATATTTGCATATACGATACAAGGCTGTTAGAGAGATAATTAGAATTAATTTGACT |
| | | GTAAACACAAAGATATTAGTACAAAATACGTGACGTAGAAAGTAATAATTTCTTGGGTAGTTTGCAG |
| | | TTTTAAAATTATGTTTTAAAATGGACTATCATATGCTTACCGTAACTTGAAAGTATTTCGATTTCTTGG |
| | | CTTTATATATCTTGTGGAAAGGACGAAACACCGTTTGTGACCCACGCCTGCTCCCGTCATAGTTCCAT |
| | | TAAAGCCAAAAGTGGCTTTGATGTTTCTATGATAAGGGTTTCGACCCGTGGCGTCGGGGATCGCCTG |
| | | CCCATTGAAATGGGCTTCTCCCCATTTATTTTTTTGAGCTCGAATTCGGGCGGAGTTAGGGCGGAGCC |
| | | AATCAGCGTGCGCCGTTCCGAAAGTTGCCTTTTATGGCTGGGCGGAGAATGGGCGGTGAACGCCGAT |
| | | GATTATATAAGGACGCGCCGGGTGTGGCACAGCTAGTTCCGTCGCAGCCGGGATTTGGGTCGCGGTT |
| | | CTTGTTTGTGGATCCCTGTGATCGTCACTTGACAGCGGCCGCGCCACCATGGCCCCTAAGAAGAAAA |
| | | GAAAGGTGATGAGAGAGCTGGACTACAGAATTGGCCTGGACATCGGCACCAACAGCATCGGATGGG |
| | | GCGTGATCGAGCTGTCCTGGAACAAAGACCGGGAGAGATACGAGAAGGTCAGAATCGTGGATCAAG |
| | | GCGTGAGAATGTTCGACAGAGCCGAGATGCCCAAGACAGGCGCCAGCTTAGCTGAACCCAGAAGAA |
| | | TCGCCAGATCCAGCAGACGGAGACTGAATCGCAAGTCCCAGAGAAAGAAAAACATCCGGAACCTGC |
| | | TGGTGCAACACGGCGTGATCACACAGGAGGAACTGGATAGCCTGTACCCCCTGAGCAAAAAGAGCA |
| | | TGGACATCTGGGGCATTCGGCTCGACGGCCTGGACAGACTCCTCAATCATTTCGAGTGGGCCAGACT |
| | | GCTGATCCACCTGGCTCAGAGACGGGGCTTTAAGTCCAACAGAAAGAGTGAACTGAAAGATACAGA |
| | | GACAGGCAAGGTGCTGAGCAGCATCCAACTGAACGAGAAACGGCTGAGCTTGTATAGAACCGTGGG |
| | | CGAGATGTGGATGAAGGACCCCGACTTCTCTAAATACGATAGGAAGAGAAATAGCCCCAACGAATA |
| | | CGTGTTCAGCGTGTCTAGAGCCGAGCTGGAAAAGGAAATCGTGACCCTGTTCGCCGCCCAGCGGAGA |
| | | TTCCAGAGCCCTTACGCCAGCAAAGATCTGCAGGAGACATATCTGCAGATCTGGACCCACCAACTGC |
| | | CTTTCGCCAGCGGCAATGCCATCCTGAACAAGGTCGGATACTGCTCCCTGTTGAAAGGCAAAGAAAG |
| | | AAGGATTCCCAAGGCTACATACACCTTCCAATACTTCTCTGCTCTGGACCAGGTGAATCGGACCAGA |
| | | CTGGGACCTGATTTCCAGCCCTTCACCAAGGAGCAACGGGAAATTATCTTGAACAACATGTTCCAGA |
| | | GGACAGATTACTACAAGAAGAAAACCATCCCCGAGGTGACCTACTATGACATACGGAAGTGGCTGG |
| | | AATTGGACGAGACAATTCAGTTCAAGGGCCTGAACTACGACCCTAACGAGGAACTGAAGAAGATCG |
| | | AGAAGAAGCCTTTTATCAATCTGAAGGCCTTCTACGAGATCAACAAGGTGGTGGCCAACTACAGCGA |
| | | AAGAACCAACGAGACCTTCTCCACCCTGGACTACGACGGCATCGGCTACGCCCTGACCGTGTACAAA |
| | | ACCGACAAGGATATCCGCAGCTACCTGAAGAGCAGTCACAACCTACCTAAGAGATGCTACGACGAC |
| | | CAACTGATCGAGGAACTGCTGAGCCTGAGCTACACAAAGTTCGGCCACCTGTCCCTGAAAGCCATCA |
| | | ACCACGTGCTGTCTATCATGCAGAAGGGCAATACCTACAAGGAAGCCGTGGACCAACTGGGCTACG |
| | | ACACCAGCGGCCTTAAGAAGGAGAAGAGGTCCAAGTTCCTGCCACCTATTTCTGATGAAATCACGAA |
| | | TCCAATCGTGAAAAGGGCCCTGACCCAGGCCAGAAAAGTGGTGAACGCCATAATTAGAAGACACGG |
| | | ATCTCCTCACTCCGTGCACATCGAGCTGGCCAGAGAGCTGAGCAAGAACCACGACGAGCGGACAAA |
| | | GATCGTCAGCGCCCAGGATGAAAACTACAAGAAAAACAAGGGCGCTATCAGCATCCTGTCTGAGCA |
| | | CGGCATCCTGAACCCTACAGGCTACGACATCGTGAGATACAAACTGTGGAAGGAGCAGGGCGAACG |
| | | GTGCGCCTACAGCCTGAAGGAAATCCCTGCCGATACATTTTTCAACGAGCTGAAGAAGGAACGCAAC |
| | | GGCGCCCCTATCCTTGAAGTGGACCACATCCTGCCCTACAGCCAGTCCTTCATCGACTCCTACCACAA |
| | | CAAGGTCCTGGTGTACAGCGACGAAAACCGGAAAAAGGGCAACAGAATCCCTTATACCTACTTCCTG |
| | | GAAACCAACAAGGATTGGGAGGCCTTTGAGCGGTACGTGCGGAGCAACAAATTCTTCTCCAAGAAA |
| | | AAGCGAGAGTACCTTCTGAAGCGGGCTTATCTGCCTAGAGAATCTGAGCTGATCAAAGAACGCCACC |
| | | TGAACGACACCAGATACGCCTCTACCTTCCTGAAGAACTTCATCGAGCAGAACCTGCAGTTCAAGGA |
| | | AGCCGAGGACAACCCCAGAAAAAGACGGGTGCAAACCGTGAACGGCGTTATCACCGCCCACTTCAG |
| | | AAAGCGGTGGGGCCTGGAGAAGGACCGGCAGGAGACATACCTCCATCACGCTATGGACGCCATCAT |
| | | CGTGGCTTGTACAGACCACCACATGGTCACCAGAGTGACCGAGTACTATCAGATCAAGGAAAGCAA |
| | | CAAGAGCGTGAAGAAGCCCTATTTTCCTATGCCTTGGGAAGGCTTCCGGGACGAGCTGCTGAGCCAC |
| | | TTGGCTTCTCAGCCTATCGCCAAGAAAATCAGCGAGGAACTGAAGGCCGGCTACCAGAGCCTGGACT |
| | | ACATCTTCGTGTCCAGAATGCCTAAGAGAAGCATTACAGGCGCTGCTCATAAGCAGACCATCATGCG |
| | | GAAGGGAGGAATTGACAAGAAGGGCAAAACAATCATCATCGAACGGCTGCACCTGAAGGATATCAA |
| | | GTTCGACGAGAACGGAGATTTCAAGATGGTGGGCAAGGAACAGGACATGGCCACATACGAAGCTAT |
| | | TAAACAGAGATACCTGGAGCACGGCAAGAATAGCAAGAAGGCCTTCGAGACCCCTCTGTACAAGCC |
| | | CAGCAAAAAGGGCACAGGTAACCTGATCAAGCGGGTGAAGGTGGAAGGACAGGCCAAGAGCTTTGT |
| | | GAGGGAAGTGAACGGCGGAGTGGCCCAAAATGGCGATCTGGTTAGAGTTGATTTGTTTGAGAAGGA |
| | | TGATAAGTACTACATGGTCCCCATCTACGTGCCAGACACCGTGTGTAGCGAGCTGCCCAAAAAGGTG |
| | | GTCGCCAGCTCTAAGGGCTATGAGCAGTGGCTGACACTGGATAACAGCTTCACCTTTAAGTTCAGCC |
| | | TGTACCCTTATGATCTGGTGCGGCTGGTCAAGGGAGATGAGGATCGGTTCCTGTACTTTGGCACCCTG |
| | | GACATCGACAGCGACAGACTTAACTTCAAGGACGTGAACAAGCCAAGCAAGAAGAACGAGTACCGG |
| | | TACAGCTTGAAAACCATCGAGGACTTGGAGAAGTACGAGGTGGGCGTGCTGGGCGATCTAAGACTG |
| | | GTCCGGAAGGAAACTCGAAGAAACTTCCACAGCGGCGGAAGCAAAAGACCTGCCGCTACAAAGAAG |
| | | GCCGGCCAGGCCAAGAAAAAGAAGTAAGCTAGCTTGACTGACTGAGATACAGCGTACCTTCAGCTC |
| | | ACAGACATGATAAGATACATTGATGAGTTTGGACAAACCACAACTAGAATGCAGTGAAAAAAATGC |
| | | TTTATTTGTGAAATTTGTGATGCTATTGCTTTATTTGTAACCATTATAAGCTGCAATAAACAAGTTAA |
| | | CAACAACAATTGCATTCATTTTATGTTTCAGGTTCAGGGGGAGGTGTGGGAGGTTTTTTAAAGCAAG |
| | | TAAAAGCTTGGTAGGAACCCCTAGTGATGGAGTTGGCCACTCCCTCTCTGCGCGCTCGCTCGCTCACT |
| | | GAGGCCGGGCGACCAAAGGTCGCCCGACGCCCGGGCTTTGCCCGGGCGGCCTCAGTGAGCGAGCGA |
| | | GCGCGCAGAGGCATTTAATTAAGCAAGCTGTCATAGCTGTTTCCTGTGTGAAATTGTTATCCGCTCAC |
| | | AATTCCACACAACATACGAGCCGGAAGCATAAAGTGTAAAGCCTGGGGTGCCTAATGAGTGAGCTA |
| | | ACTCACATTAATTGCGTTGCGCTCACTGCCCGCTTTCCAGTCGGGAAACCTGTCGTGCCAGCTGCATT |
| | | AATGAATCGGCCAACGCGCGGGGAGAGGCGGTTTGCGTATTGGGCGCTCTTCCGCTTCCTCGCTCAC |
| | | TGACTCGCTGCGCTCGGTCGTTCGGCTGCGGCGAGCGGTATCAGCTCACTCAAAGGCGGTAATACGG |
| | | TTATCCACAGAATCAGGGGATAACGCAGGAAAGAACATGTGAGCAAAAGGCCAGCAAAAGGCCAG |
| | | GAACCGTAAAAAGGCCGCGTTGCTGGCGTTTTTCCATAGGCTCCGCCCCCCTGACGAGCATCACAAA |
| | | AATCGACGCTCAAGTCAGAGGTGGCGAAACCCGACAGGACTATAAAGATACCAGGCGTTTCCCCCT |
| | | GGAAGCTCCCTCGTGCGCTCTCCTGTTCCGACCCTGCCGCTTACCGGATACCTGTCCGCCTTTCTCCCT |
| | | TCGGGAAGCGTGGCGCTTTCTCATAGCTCACGCTGTAGGTATCTCAGTTCGGTGTAGGTCGTTCGCTC |
| | | CAAGCTGGGCTGTGTGCACGAACCCCCCGTTCAGCCCGACCGCTGCGCCTTATCCGGTAACTATCGT |
| | | CTTGAGTCCAACCCGGTAAGACACGACTTATCGCCACTGGCAGCAGCCACTGGTAACAGGATTAGCA |
| | | GAGCGAGGTATGTAGGCGGTGCTACAGAGTTCTTGAAGTGGTGGCCTAACTACGGCTACACTAGAAG |
| | | AACAGTATTTGGTATCTGCGCTCTGCTGAAGCCAGTTACCTTCGGAAAAAGAGTTGGTAGCTCTTGAT |
| | | CCGGCAAACAAACCACCGCTGGTAGCGGTGGTTTTTTTGTTTGCAAGCAGCAGATTACGCGCAGAAA |
| | | AAAAGGATCTCAAGAAGATCCTTTGATCTTTTCTACGGGGTCTGACGCTCAGTGGAACGAAAACTCA |
| | | CGTTAAGGGATTTTGGTCATGAGATTATCAAAAAGGATCTTCACCTAGATCCTTTTAAATTAAAAATG |
| | | AAGTTTTAAATCAAGCCCAATCTGAATAATGTTACAACCAATTAACCAATTCTGATTAGAAAAACTC |
| | | ATCGAGCATCAAATGAAACTGCAATTTATTCATATCAGGATTATCAATACCATATTTTTGAAAAAGC |
| | | CGTTTCTGTAATGAAGGAGAAAACTCACCGAGGCAGTTCCATAGGATGGCAAGATCCTGGTATCGGT |
| | | CTGCGATTCCGACTCGTCCAACATCAATACAACCTATTAATTTCCCCTCGTCAAAAATAAGGTTATCA |
| | | AGTGAGAAATCACCATGAGTGACGACTGAATCCGGTGAGAATGGCAAAAGTTTATGCATTTCTTTCC |
| | | AGACTTGTTCAACAGGCCAGCCATTACGCTCGTCATCAAAATCACTCGCATCAACCAAACCGTTATT |
| | | CATTCGTGATTGCGCCTGAGCGAGACGAAATACGCGATCGCTGTTAAAAGGACAATTACAAACAGG |
| | | AATCGAATGCAACCGGCGCAGGAACACTGCCAGCGCATCAACAATATTTTCACCTGAATCAGGATAT |
| | | TCTTCTAATACCTGGAATGCTGTTTTTCCGGGGATCGCAGTGGTGAGTAACCATGCATCATCAGGAGT |
| | | ACGGATAAAATGCTTGATGGTCGGAAGAGGCATAAATTCCGTCAGCCAGTTTAGTCTGACCATCTCA |
| | | TCTGTAACATCATTGGCAACGCTACCTTTGCCATGTTTCAGAAACAACTCTGGCGCATCGGGCTTCCC |
| | | ATACAAGCGATAGATTGTCGCACCTGATTGCCCGACATTATCGCGAGCCCATTTATACCCATATAAA |
| | | TCAGCATCCATGTTGGAATTTAATCGCGGCCTCGACGTTTCCCGTTGAATATGGCTCATAACACCCCT |
| | | TGTATTACTGTTTATGTAAGCAGACAGTTTTATTGTTCATGATGATATATTTTTATCTTGTGCAATGTA |
| | | ACATCAGAGATTTTGAGACACGGGCCAGAGCTGCATCGCGCGTTTCGGTGATGACGGTGAAAACCTC |
| | | TGACACATGCAGCTCCCGGAGACGGTCACAGCTTGTCTGTAAGCGGATGCCGGGAGCAGACAAGCC |
| | | CGTCAGGGCGCGTCAGCGGGTGTTGGCGGGTGTCGGGGCTGGCTTAACTATGCGGCATCAGAGCAG |
| | | ATTGTACTGAGAGTGCACGATATCGGGTCC |
| 32 | pAAV-hU6(318 bp)- | GTGCACGATATCGGGTCCCCAATTGCTGCGCGCTCGCTCGCTCACTGAGGCCGCCCGGGCAAAGCCC |
| | SGN004282-JeT- | GGGCGTCGGGCGACCTTTGGTCGCCCGGCCTCAGTGAGCGAGCGAGCGCGCAGAGAGGGAGTGGCC |
| | APG05586- | AACTCCATCACTAGGGGTTCCTCGTCGACAATGCACGCGTTCGAAGTACTCATGTACAAAAAAGCAG |
| | SV40pA(179 bp) | GCTTTAAAGGAACCAATTCAGTCGACTGGATCCGGTACCAAGGTCGGGCAGGAAGAGGGCCTATTTC |
| | | CCATGATTCCTTCATATTTGCATATACGATACAAGGCTGTTAGAGAGATAATTAGAATTAATTTGACT |
| | | GTAAACACAAAGATATTAGTACAAAATACGTGACGTAGAAAGTAATAATTTCTTGGGTAGTTTGCAG |
| | | TTTTAAAATTATGTTTTAAAATGGACTATCATATGCTTACCGTAACTTGAAAGTATTTCGATTTCTTGG |
| | | CTTTATATATCTTGTGGAAAGGACGAAACACCGACCGGCCTCCAGGATGAAGTGCACAGTTATTGTA |
| | | CTCAAAGGAATCTACAATAATAAGGCATCTTGCCGAATTTACCGCCCTACATATGTAGGGCGGTTTTT |
| | | TTTTGTCTAGACAAGTTGGAGCTCGAATTCGGGCGGAGTTAGGGCGGAGCCAATCAGCGTGCGCCGT |
| | | TCCGAAAGTTGCCTTTTATGGCTGGGCGGAGAATGGGCGGTGAACGCCGATGATTATATAAGGACGC |
| | | GCCGGGTGTGGCACAGCTAGTTCCGTCGCAGCCGGGATTTGGGTCGCGGTTCTTGTTTGTGGATCCCT |
| | | GTGATCGTCACTTGACAGCGGCCGCGCCACCATGGCCCCTAAGAAGAAAAGAAAGGTGATGTACAG |
| | | TATAGGGTTAGATTTAGGGATAAGTAGTGTTGGTTGGTCAGTTATTGATGAAGAAACAGGAAAAATT |
| | | GTGGACTTAGGTGTTCGCTTATTTAGTGCAAAAAATAGTGAGAAAAATTTAGAACGACGAACTAGTC |
| | | GTGGTGCTCGTCGCTTAATACGACGGAAAACAAATCGCTTAAAAGATGCGAAGAAATTACTTGAAGC |
| | | GATTGGCTTTTATGAAGACAAGGCATTAAAAAATGTTTGTCCTTATCAATTAAGAGTAAAAGGTTTG |
| | | ACTGAGGGCTTAACCAAGGGAGAGCTTTACAAAGTTGTGCTTCATATTGTCAAAAAACGTGGGATTA |
| | | GTTATTTGGATGAGGATGATGCTGAAGCCGCAAAAGAAAGCCAAGATTACAAAGAACAAGTCAGAA |
| | | AAAATGCTCAATTGTTAACAAAGTATACGCCTGGTCAAATTCAATTGCAGCGACTGAAAGAAAACAA |
| | | TCGAGTGAAAACAGGGATTAATGGTCAAGGCCATTATCAGTTAAATGTCTTTAAAGTTTCTGCTTATG |
| | | CTGATGAATTGGCTACCATCCTGAAAACTCAACAGGCCTTGTATCCAAATGAATTAACAGATGATTG |
| | | GATTGCGTTATTTGTGCAACCAGGTATTGCAGAGAATGCCGGGTTAATTTATCGCAAACGCCCTTATT |
| | | ATCATGGACCAGGAAATGAAGCTAATAATAGCCCTTATGGTCGTTGGTCTGATTTCCAAAAAACAGG |
| | | ACAACCGGCAGCAAATATTTTTGATAAATTGATTGGCAAAGACTTTCAAGGTGAACTACGAGCAAGC |
| | | GGCTTAAGTTTATCTGCACAACAATATAATTTACTGAATGATTTAACCAATTTAAAAATTGATGGGG |
| | | AAGTTTCACTTTCTCCTGAACAAAAGGAATTCATTTTAACAGAACTAATGACGAAAGAATTTGCTCG |
| | | TTTTGGTGTCAACGATATCGCTAAATTATTAGGTGTAAAAAAAGAGCAACTCAGCGGCTGGCGATTG |
| | | GATAAAAAAGGTAAACCAGAAATTCATACATTAAAAGGCTATCGCAACTGGCGGAAAATTTTTGCG |
| | | GAAGCAGGGATCGATTTGGCTACTTTACCAACAGAAACGATTGACTGTTTAGCGAAAGTGCTAACAT |
| | | TGAATACCGAACGCGAAGGGGTTGAAAACACATTAGCTTTTGAACTTCCTGAGTTAGCAGAACCGGT |
| | | GAAGTCACTGGTTTTGGATCATTACAAAGAATTAAGTCAAAGTATTAGCACGCAAGCATGGCACCGT |
| | | TTTTCTCTAAAAACGTTACACCTATTAATTCCAGAACTAATCAAATCCACATCGGAACAAAATACGCT |
| | | CTTAGAACAGTTTCAGTTAAAAGCAGGTGTCAGAAAACGCTACAGTGATTATAAAAAGTTACCAACC |
| | | AAAGAGGTTCTAGCAGAAATTTATAATCCTACAGTGAACAAAACAGTCAGTCAAGCCTTCAAAGTTA |
| | | TGGATGCTTTGTTGGAAAAATATGGCAAAGACCAAATCCATTATATTACCGTCGAAATGCCACGTGA |
| | | TGATAACGAAGAAGAAGAACGAAAACGGATAAAAGAGCTACAAACAAAGAATAGTCAACGAAAAA |
| | | ATGACAGTCAACAGTACTTTCTACAAAAGTCAGGTTGGAGTCAAGAAAAATTTCAAGCAACCATTCA |
| | | CAAAAATCGTCGTTTTTTAGCCAAATTGTTATACTACTTTGAACAAGACGGGGTATGTGCCTATACGG |
| | | GAAATCCTATTTCACCTGAATTATTAGTCAGTGATTCAACAGAAATTGACCATATTATTCCAATTTCG |
| | | ATAAGTCTAGATGATTCTATTAATAATAAAGTCTTGGTTTTAAGTCACGCGAATCAAGTCAAAGGAC |
| | | AACAAACACCTTATGATGCTAGGATGGCTGGCGCATTTAACAAAATTAATGGGAAATTTAGCAACTG |
| | | GGATGAGTATCAAAAGTGGGTAGAAAGTCGTCCGTTCAGTCGTAAAAAAGTAAATAATTTATTGGAA |
| | | ACACGGAATATTTTTGATAGTGAACAAGTTCAAAAGTTTCTGTCACGAAATCTTAACGATACGCGTT |
| | | ACGCTAGTCGTTTGGTTTTAAATACGTTGCAAAGCTTTTTTGAAAATCAAGACACAATTGTTCGAGTA |
| | | GTAAATGGTAGTTTCACTCATACCTTACGTAAAAAATGGGGTGCTGACTTAGATAAAACTCGTGAAA |
| | | CCCATCATCATCATGCCGTTGATGCGACACTTTGTGCAGTGACACCATTTGTGAAAGTCAGTCGTTAT |
| | | CACTATGCGGTCAACGAAGAAACCGGTGAAAAGTTCATGCGTGAAATTGATGTTGAAACTGGTGAA |
| | | ATTCTCGATGAAATTCCGTATAGAGAATACAAAAAAGCAAAACATTATGAACGAAAAACGTACCAA |
| | | GTCAAATGGTCAAATTTTCGGGAACAATTAAAACCAATAACGATTCATCCAAAAATTAAATTTTCTC |
| | | ATCAAGTGGATCGCAAAGCTAATCGCAAACTCAGTGATGCAACCATTTACTCTGTTCGTGAAAAAAC |
| | | AGAAGTCAAAACACTAAAAAGTGGTAAAGAAAAAATAACGACAGATGAATATACCATCGGAAAAA |
| | | TTAAAGATATTTACACGGTAGATGGTTGGGAAGCATTTAAGAAAAAACAAGACAAACTATTAATGA |
| | | AAGAGTTTGATGAAAAAACGTACGAATTATTAGTGACCATTGCAGCAACAACGCCAGATTTTCAAGA |
| | | AGTGGAAGAAAAAAATGGCAAGGTGAAACGTGTGAAGCGCTCACCATTTGCTGTTTACTGCGAAGA |
| | | AAATGGGATTCCAGCGATTCGAAAATATGCGAAAAAAAATAACGGTCCAGTTATCCGTAGCTTAAA |
| | | ATATTACGATGGCAAATTAAACAAACATATCAATATTACCAAAGACGAAAAAGGTCGACCAGTTGA |
| | | ACAAACCAAAAATGGTCGCAAAGTTACTTTACAAAGTCTAAAACCCTATCGTTACGATATTTATCAA |
| | | GATTTAGAAACGAAGGCTTACTATACAGTGCAACTATATTATTCTGACTTGCGTTTTGTGGAAGGTGA |
| | | GTATGGAATTACAGAAAAAGAATACATGAAGAAAGTCGCTGAGCAGACAAAGGGACAAGTCGTACG |
| | | TTTCTGCTTCTCTTTACAAAAAAATGATGGTTTGGAAATCGAATGGAAAGATAGCCAACGCTATGAT |
| | | GTGCGTTTTTATAATTTCCAAAGTGCTAATAGCATTAACTTTAAAGGGTTAGAACAAGAAATGATAC |
| | | CAGCAGAAAATCAATTTAAGCAAAAACCTTATAATAATGGAGCCATCAATTTAAATATTGCTAAATA |
| | | TGGCAAAGAAGGCAAAAAGTTACGAAAATTCAATACAGATATTTTAGGAAAGAAACATCATCTATC |
| | | TTATGAAAAAGAACCAAAGAATATTATTAAAAGCGGCGGAAGCAAAAGACCTGCCGCTACAAAGAA |
| | | GGCCGGCCAGGCCAAGAAAAAGAAGTAAGCTAGCTTGACTGACTGAGATACAGCGTACCTTCAGCT |
| | | CACAGACATGATAAGATACATTGATGAGTTTGGACAAACCACAACTAGAATGCAGTGAAAAAAATG |
| | | CTTTATTTGTGAAATTTGTGATGCTATTGCTTTATTTGTAACCATTATAAGCTGCAATAAACAAGTTA |
| | | ACAACAACAATTGCATTCATTTTATGTTTCAGGTTCAGGGGGAGGTGTGGGAGGTTTTTTAAAGCAA |
| | | GTAAAAGCTTGGTAGGAACCCCTAGTGATGGAGTTGGCCACTCCCTCTCTGCGCGCTCGCTCGCTCA |
| | | CTGAGGCCGGGCGACCAAAGGTCGCCCGACGCCCGGGCTTTGCCCGGGCGGCCTCAGTGAGCGAGC |
| | | GAGCGCGCAGAGGCATTTAATTAAGCAAGCTGTCATAGCTGTTTCCTGTGTGAAATTGTTATCCGCTC |
| | | ACAATTCCACACAACATACGAGCCGGAAGCATAAAGTGTAAAGCCTGGGGTGCCTAATGAGTGAGC |
| | | TAACTCACATTAATTGCGTTGCGCTCACTGCCCGCTTTCCAGTCGGGAAACCTGTCGTGCCAGCTGCA |
| | | TTAATGAATCGGCCAACGCGCGGGGAGAGGCGGTTTGCGTATTGGGCGCTCTTCCGCTTCCTCGCTC |
| | | ACTGACTCGCTGCGCTCGGTCGTTCGGCTGCGGCGAGCGGTATCAGCTCACTCAAAGGCGGTAATAC |
| | | GGTTATCCACAGAATCAGGGGATAACGCAGGAAAGAACATGTGAGCAAAAGGCCAGCAAAAGGCC |
| | | AGGAACCGTAAAAAGGCCGCGTTGCTGGCGTTTTTCCATAGGCTCCGCCCCCCTGACGAGCATCACA |
| | | AAAATCGACGCTCAAGTCAGAGGTGGCGAAACCCGACAGGACTATAAAGATACCAGGCGTTTCCCC |
| | | CTGGAAGCTCCCTCGTGCGCTCTCCTGTTCCGACCCTGCCGCTTACCGGATACCTGTCCGCCTTTCTCC |
| | | CTTCGGGAAGCGTGGCGCTTTCTCATAGCTCACGCTGTAGGTATCTCAGTTCGGTGTAGGTCGTTCGC |
| | | TCCAAGCTGGGCTGTGTGCACGAACCCCCCGTTCAGCCCGACCGCTGCGCCTTATCCGGTAACTATC |
| | | GTCTTGAGTCCAACCCGGTAAGACACGACTTATCGCCACTGGCAGCAGCCACTGGTAACAGGATTAG |
| | | CAGAGCGAGGTATGTAGGCGGTGCTACAGAGTTCTTGAAGTGGTGGCCTAACTACGGCTACACTAGA |
| | | AGAACAGTATTTGGTATCTGCGCTCTGCTGAAGCCAGTTACCTTCGGAAAAAGAGTTGGTAGCTCTT |
| | | GATCCGGCAAACAAACCACCGCTGGTAGCGGTGGTTTTTTTGTTTGCAAGCAGCAGATTACGCGCAG |
| | | AAAAAAAGGATCTCAAGAAGATCCTTTGATCTTTTCTACGGGGTCTGACGCTCAGTGGAACGAAAAC |
| | | TCACGTTAAGGGATTTTGGTCATGAGATTATCAAAAAGGATCTTCACCTAGATCCTTTTAAATTAAAA |
| | | ATGAAGTTTTAAATCAAGCCCAATCTGAATAATGTTACAACCAATTAACCAATTCTGATTAGAAAAA |
| | | CTCATCGAGCATCAAATGAAACTGCAATTTATTCATATCAGGATTATCAATACCATATTTTTGAAAAA |
| | | GCCGTTTCTGTAATGAAGGAGAAAACTCACCGAGGCAGTTCCATAGGATGGCAAGATCCTGGTATCG |
| | | GTCTGCGATTCCGACTCGTCCAACATCAATACAACCTATTAATTTCCCCTCGTCAAAAATAAGGTTAT |
| | | CAAGTGAGAAATCACCATGAGTGACGACTGAATCCGGTGAGAATGGCAAAAGTTTATGCATTTCTTT |
| | | CCAGACTTGTTCAACAGGCCAGCCATTACGCTCGTCATCAAAATCACTCGCATCAACCAAACCGTTA |
| | | TTCATTCGTGATTGCGCCTGAGCGAGACGAAATACGCGATCGCTGTTAAAAGGACAATTACAAACAG |
| | | GAATCGAATGCAACCGGCGCAGGAACACTGCCAGCGCATCAACAATATTTTCACCTGAATCAGGATA |
| | | TTCTTCTAATACCTGGAATGCTGTTTTTCCGGGGATCGCAGTGGTGAGTAACCATGCATCATCAGGAG |
| | | TACGGATAAAATGCTTGATGGTCGGAAGAGGCATAAATTCCGTCAGCCAGTTTAGTCTGACCATCTC |
| | | ATCTGTAACATCATTGGCAACGCTACCTTTGCCATGTTTCAGAAACAACTCTGGCGCATCGGGCTTCC |
| | | CATACAAGCGATAGATTGTCGCACCTGATTGCCCGACATTATCGCGAGCCCATTTATACCCATATAA |
| | | ATCAGCATCCATGTTGGAATTTAATCGCGGCCTCGACGTTTCCCGTTGAATATGGCTCATAACACCCC |
| | | TTGTATTACTGTTTATGTAAGCAGACAGTTTTATTGTTCATGATGATATATTTTTATCTTGTGCAATGT |
| | | AACATCAGAGATTTTGAGACACGGGCCAGAGCTGCATCGCGCGTTTCGGTGATGACGGTGAAAACCT |
| | | CTGACACATGCAGCTCCCGGAGACGGTCACAGCTTGTCTGTAAGCGGATGCCGGGAGCAGACAAGC |
| | | CCGTCAGGGCGCGTCAGCGGGTGTTGGCGGGTGTCGGGGCTGGCTTAACTATGCGGCATCAGAGCAG |
| | | ATTGTACTGAGAGTGCACGATATCGGGTCC |
| 33 | pAAV-hU6(318 bp)- | GTGCACGATATCGGGTCCCCAATTGCTGCGCGCTCGCTCGCTCACTGAGGCCGCCCGGGCAAAGCCC |
| | SGN004282-hSyn- | GGGCGTCGGGCGACCTTTGGTCGCCCGGCCTCAGTGAGCGAGCGAGCGCGCAGAGAGGGAGTGGCC |
| | APG05586- | AACTCCATCACTAGGGGTTCCTCGTCGACAATGCACGCGTTCGAAGTACTCATGTACAAAAAAGCAG |
| | SV40pA(179 bp) | GCTTTAAAGGAACCAATTCAGTCGACTGGATCCGGTACCAAGGTCGGGCAGGAAGAGGGCCTATTTC |
| | | CCATGATTCCTTCATATTTGCATATACGATACAAGGCTGTTAGAGAGATAATTAGAATTAATTTGACT |
| | | GTAAACACAAAGATATTAGTACAAAATACGTGACGTAGAAAGTAATAATTTCTTGGGTAGTTTGCAG |
| | | TTTTAAAATTATGTTTTAAAATGGACTATCATATGCTTACCGTAACTTGAAAGTATTTCGATTTCTTGG |
| | | CTTTATATATCTTGTGGAAAGGACGAAACACCGACCGGCCTCCAGGATGAAGTGCACAGTTATTGTA |
| | | CTCAAAGGAATCTACAATAATAAGGCATCTTGCCGAATTTACCGCCCTACATATGTAGGGCGGTTTTT |
| | | TTTTGTCTAGACAAGTTGGAGCTCAGTGCAAGTGGGTTTTAGGACCAGGATGAGGCGGGGTGGGGGT |
| | | GCCTACCTGACGACCGACCCCGACCCACTGGACAAGCACCCAACCCCCATTCCCCAAATTGCGCATC |
| | | CCCTATCAGAGAGGGGGAGGGGAAACAGGATGCGGCGAGGCGCGTGCGCACTGCCAGCTTCAGCAC |
| | | CGCGGACAGTGCCTTCGCCCCCGCCTGGCGGCGCGCGCCACCGCCGCCTCAGCACTGAAGGCGCGCT |
| | | GACGTCACTCGCCGGTCCCCCGCAAACTCCCCTTCCCGGCCACCTTGGTCGCGTCCGCGCCGCCGCCG |
| | | GCCCAGCCGGACCGCACCACGCGAGGCGCGAGATAGGGGGGCACGGGCGCGACCATCTGCGCTGCG |
| | | GCGCCGGCGACTCAGCGCTGCCTCAGTCTGCGGTGGGCAGCGGAGGAGTCGTGTCGTGCCTGAGAGC |
| | | GCAGTCGAGAAGGTACCGGATCCCCGGGTACCGGTCGCCACCATGGCCCCTAAGAAGAAAAGAAAG |
| | | GTGATGTACAGTATAGGGTTAGATTTAGGGATAAGTAGTGTTGGTTGGTCAGTTATTGATGAAGAAA |
| | | CAGGAAAAATTGTGGACTTAGGTGTTCGCTTATTTAGTGCAAAAAATAGTGAGAAAAATTTAGAACG |
| | | ACGAACTAGTCGTGGTGCTCGTCGCTTAATACGACGGAAAACAAATCGCTTAAAAGATGCGAAGAA |
| | | ATTACTTGAAGCGATTGGCTTTTATGAAGACAAGGCATTAAAAAATGTTTGTCCTTATCAATTAAGA |
| | | GTAAAAGGTTTGACTGAGGGCTTAACCAAGGGAGAGCTTTACAAAGTTGTGCTTCATATTGTCAAAA |
| | | AACGTGGGATTAGTTATTTGGATGAGGATGATGCTGAAGCCGCAAAAGAAAGCCAAGATTACAAAG |
| | | AACAAGTCAGAAAAAATGCTCAATTGTTAACAAAGTATACGCCTGGTCAAATTCAATTGCAGCGACT |
| | | GAAAGAAAACAATCGAGTGAAAACAGGGATTAATGGTCAAGGCCATTATCAGTTAAATGTCTTTAA |
| | | AGTTTCTGCTTATGCTGATGAATTGGCTACCATCCTGAAAACTCAACAGGCCTTGTATCCAAATGAAT |
| | | TAACAGATGATTGGATTGCGTTATTTGTGCAACCAGGTATTGCAGAGAATGCCGGGTTAATTTATCG |
| | | CAAACGCCCTTATTATCATGGACCAGGAAATGAAGCTAATAATAGCCCTTATGGTCGTTGGTCTGAT |
| | | TTCCAAAAAACAGGACAACCGGCAGCAAATATTTTTGATAAATTGATTGGCAAAGACTTTCAAGGTG |
| | | AACTACGAGCAAGCGGCTTAAGTTTATCTGCACAACAATATAATTTACTGAATGATTTAACCAATTT |
| | | AAAAATTGATGGGGAAGTTTCACTTTCTCCTGAACAAAAGGAATTCATTTTAACAGAACTAATGACG |
| | | AAAGAATTTGCTCGTTTTGGTGTCAACGATATCGCTAAATTATTAGGTGTAAAAAAAGAGCAACTCA |
| | | GCGGCTGGCGATTGGATAAAAAAGGTAAACCAGAAATTCATACATTAAAAGGCTATCGCAACTGGC |
| | | GGAAAATTTTTGCGGAAGCAGGGATCGATTTGGCTACTTTACCAACAGAAACGATTGACTGTTTAGC |
| | | GAAAGTGCTAACATTGAATACCGAACGCGAAGGGGTTGAAAACACATTAGCTTTTGAACTTCCTGAG |
| | | TTAGCAGAACCGGTGAAGTCACTGGTTTTGGATCATTACAAAGAATTAAGTCAAAGTATTAGCACGC |
| | | AAGCATGGCACCGTTTTTCTCTAAAAACGTTACACCTATTAATTCCAGAACTAATCAAATCCACATCG |
| | | GAACAAAATACGCTCTTAGAACAGTTTCAGTTAAAAGCAGGTGTCAGAAAACGCTACAGTGATTATA |
| | | AAAAGTTACCAACCAAAGAGGTTCTAGCAGAAATTTATAATCCTACAGTGAACAAAACAGTCAGTC |
| | | AAGCCTTCAAAGTTATGGATGCTTTGTTGGAAAAATATGGCAAAGACCAAATCCATTATATTACCGT |
| | | CGAAATGCCACGTGATGATAACGAAGAAGAAGAACGAAAACGGATAAAAGAGCTACAAACAAAGA |
| | | ATAGTCAACGAAAAAATGACAGTCAACAGTACTTTCTACAAAAGTCAGGTTGGAGTCAAGAAAAAT |
| | | TTCAAGCAACCATTCACAAAAATCGTCGTTTTTTAGCCAAATTGTTATACTACTTTGAACAAGACGGG |
| | | GTATGTGCCTATACGGGAAATCCTATTTCACCTGAATTATTAGTCAGTGATTCAACAGAAATTGACCA |
| | | TATTATTCCAATTTCGATAAGTCTAGATGATTCTATTAATAATAAAGTCTTGGTTTTAAGTCACGCGA |
| | | ATCAAGTCAAAGGACAACAAACACCTTATGATGCTAGGATGGCTGGCGCATTTAACAAAATTAATGG |
| | | GAAATTTAGCAACTGGGATGAGTATCAAAAGTGGGTAGAAAGTCGTCCGTTCAGTCGTAAAAAAGT |
| | | AAATAATTTATTGGAAACACGGAATATTTTTGATAGTGAACAAGTTCAAAAGTTTCTGTCACGAAAT |
| | | CTTAACGATACGCGTTACGCTAGTCGTTTGGTTTTAAATACGTTGCAAAGCTTTTTTGAAAATCAAGA |
| | | CACAATTGTTCGAGTAGTAAATGGTAGTTTCACTCATACCTTACGTAAAAAATGGGGTGCTGACTTA |
| | | GATAAAACTCGTGAAACCCATCATCATCATGCCGTTGATGCGACACTTTGTGCAGTGACACCATTTGT |
| | | GAAAGTCAGTCGTTATCACTATGCGGTCAACGAAGAAACCGGTGAAAAGTTCATGCGTGAAATTGAT |
| | | GTTGAAACTGGTGAAATTCTCGATGAAATTCCGTATAGAGAATACAAAAAAGCAAAACATTATGAA |
| | | CGAAAAACGTACCAAGTCAAATGGTCAAATTTTCGGGAACAATTAAAACCAATAACGATTCATCCAA |
| | | AAATTAAATTTTCTCATCAAGTGGATCGCAAAGCTAATCGCAAACTCAGTGATGCAACCATTTACTCT |
| | | GTTCGTGAAAAAACAGAAGTCAAAACACTAAAAAGTGGTAAAGAAAAAATAACGACAGATGAATAT |
| | | ACCATCGGAAAAATTAAAGATATTTACACGGTAGATGGTTGGGAAGCATTTAAGAAAAAACAAGAC |
| | | AAACTATTAATGAAAGAGTTTGATGAAAAAACGTACGAATTATTAGTGACCATTGCAGCAACAACGC |
| | | CAGATTTTCAAGAAGTGGAAGAAAAAAATGGCAAGGTGAAACGTGTGAAGCGCTCACCATTTGCTG |
| | | TTTACTGCGAAGAAAATGGGATTCCAGCGATTCGAAAATATGCGAAAAAAAATAACGGTCCAGTTAT |
| | | CCGTAGCTTAAAATATTACGATGGCAAATTAAACAAACATATCAATATTACCAAAGACGAAAAAGG |
| | | TCGACCAGTTGAACAAACCAAAAATGGTCGCAAAGTTACTTTACAAAGTCTAAAACCCTATCGTTAC |
| | | GATATTTATCAAGATTTAGAAACGAAGGCTTACTATACAGTGCAACTATATTATTCTGACTTGCGTTT |
| | | TGTGGAAGGTGAGTATGGAATTACAGAAAAAGAATACATGAAGAAAGTCGCTGAGCAGACAAAGG |
| | | GACAAGTCGTACGTTTCTGCTTCTCTTTACAAAAAAATGATGGTTTGGAAATCGAATGGAAAGATAG |
| | | CCAACGCTATGATGTGCGTTTTTATAATTTCCAAAGTGCTAATAGCATTAACTTTAAAGGGTTAGAAC |
| | | AAGAAATGATACCAGCAGAAAATCAATTTAAGCAAAAACCTTATAATAATGGAGCCATCAATTTAA |
| | | ATATTGCTAAATATGGCAAAGAAGGCAAAAAGTTACGAAAATTCAATACAGATATTTTAGGAAAGA |
| | | AACATCATCTATCTTATGAAAAAGAACCAAAGAATATTATTAAAAGCGGCGGAAGCAAAAGACCTG |
| | | CCGCTACAAAGAAGGCCGGCCAGGCCAAGAAAAAGAAGTAAGCTAGCTTGACTGACTGAGATACAG |
| | | CGTACCTTCAGCTCACAGACATGATAAGATACATTGATGAGTTTGGACAAACCACAACTAGAATGCA |
| | | GTGAAAAAAATGCTTTATTTGTGAAATTTGTGATGCTATTGCTTTATTTGTAACCATTATAAGCTGCA |
| | | ATAAACAAGTTAACAACAACAATTGCATTCATTTTATGTTTCAGGTTCAGGGGGAGGTGTGGGAGGT |
| | | TTTTTAAAGCAAGTAAAAGCTTGGTAGGAACCCCTAGTGATGGAGTTGGCCACTCCCTCTCTGCGCG |
| | | CTCGCTCGCTCACTGAGGCCGGGCGACCAAAGGTCGCCCGACGCCCGGGCTTTGCCCGGGCGGCCTC |
| | | AGTGAGCGAGCGAGCGCGCAGAGGCATTTAATTAAGCAAGCTGTCATAGCTGTTTCCTGTGTGAAAT |
| | | TGTTATCCGCTCACAATTCCACACAACATACGAGCCGGAAGCATAAAGTGTAAAGCCTGGGGTGCCT |
| | | AATGAGTGAGCTAACTCACATTAATTGCGTTGCGCTCACTGCCCGCTTTCCAGTCGGGAAACCTGTCG |
| | | TGCCAGCTGCATTAATGAATCGGCCAACGCGCGGGGAGAGGCGGTTTGCGTATTGGGCGCTCTTCCG |
| | | CTTCCTCGCTCACTGACTCGCTGCGCTCGGTCGTTCGGCTGCGGCGAGCGGTATCAGCTCACTCAAAG |
| | | GCGGTAATACGGTTATCCACAGAATCAGGGGATAACGCAGGAAAGAACATGTGAGCAAAAGGCCAG |
| | | CAAAAGGCCAGGAACCGTAAAAAGGCCGCGTTGCTGGCGTTTTTCCATAGGCTCCGCCCCCCTGACG |
| | | AGCATCACAAAAATCGACGCTCAAGTCAGAGGTGGCGAAACCCGACAGGACTATAAAGATACCAGG |
| | | CGTTTCCCCCTGGAAGCTCCCTCGTGCGCTCTCCTGTTCCGACCCTGCCGCTTACCGGATACCTGTCC |
| | | GCCTTTCTCCCTTCGGGAAGCGTGGCGCTTTCTCATAGCTCACGCTGTAGGTATCTCAGTTCGGTGTA |
| | | GGTCGTTCGCTCCAAGCTGGGCTGTGTGCACGAACCCCCCGTTCAGCCCGACCGCTGCGCCTTATCCG |
| | | GTAACTATCGTCTTGAGTCCAACCCGGTAAGACACGACTTATCGCCACTGGCAGCAGCCACTGGTAA |
| | | CAGGATTAGCAGAGCGAGGTATGTAGGCGGTGCTACAGAGTTCTTGAAGTGGTGGCCTAACTACGGC |
| | | TACACTAGAAGAACAGTATTTGGTATCTGCGCTCTGCTGAAGCCAGTTACCTTCGGAAAAAGAGTTG |
| | | GTAGCTCTTGATCCGGCAAACAAACCACCGCTGGTAGCGGTGGTTTTTTTGTTTGCAAGCAGCAGATT |
| | | ACGCGCAGAAAAAAAGGATCTCAAGAAGATCCTTTGATCTTTTCTACGGGGTCTGACGCTCAGTGGA |
| | | ACGAAAACTCACGTTAAGGGATTTTGGTCATGAGATTATCAAAAAGGATCTTCACCTAGATCCTTTT |
| | | AAATTAAAAATGAAGTTTTAAATCAAGCCCAATCTGAATAATGTTACAACCAATTAACCAATTCTGA |
| | | TTAGAAAAACTCATCGAGCATCAAATGAAACTGCAATTTATTCATATCAGGATTATCAATACCATAT |
| | | TTTTGAAAAAGCCGTTTCTGTAATGAAGGAGAAAACTCACCGAGGCAGTTCCATAGGATGGCAAGAT |
| | | CCTGGTATCGGTCTGCGATTCCGACTCGTCCAACATCAATACAACCTATTAATTTCCCCTCGTCAAAA |
| | | ATAAGGTTATCAAGTGAGAAATCACCATGAGTGACGACTGAATCCGGTGAGAATGGCAAAAGTTTA |
| | | TGCATTTCTTTCCAGACTTGTTCAACAGGCCAGCCATTACGCTCGTCATCAAAATCACTCGCATCAAC |
| | | CAAACCGTTATTCATTCGTGATTGCGCCTGAGCGAGACGAAATACGCGATCGCTGTTAAAAGGACAA |
| | | TTACAAACAGGAATCGAATGCAACCGGCGCAGGAACACTGCCAGCGCATCAACAATATTTTCACCTG |
| | | AATCAGGATATTCTTCTAATACCTGGAATGCTGTTTTTCCGGGGATCGCAGTGGTGAGTAACCATGCA |
| | | TCATCAGGAGTACGGATAAAATGCTTGATGGTCGGAAGAGGCATAAATTCCGTCAGCCAGTTTAGTC |
| | | TGACCATCTCATCTGTAACATCATTGGCAACGCTACCTTTGCCATGTTTCAGAAACAACTCTGGCGCA |
| | | TCGGGCTTCCCATACAAGCGATAGATTGTCGCACCTGATTGCCCGACATTATCGCGAGCCCATTTATA |
| | | CCCATATAAATCAGCATCCATGTTGGAATTTAATCGCGGCCTCGACGTTTCCCGTTGAATATGGCTCA |
| | | TAACACCCCTTGTATTACTGTTTATGTAAGCAGACAGTTTTATTGTTCATGATGATATATTTTTATCTT |
| | | GTGCAATGTAACATCAGAGATTTTGAGACACGGGCCAGAGCTGCATCGCGCGTTTCGGTGATGACGG |
| | | TGAAAACCTCTGACACATGCAGCTCCCGGAGACGGTCACAGCTTGTCTGTAAGCGGATGCCGGGAGC |
| | | AGACAAGCCCGTCAGGGCGCGTCAGCGGGTGTTGGCGGGTGTCGGGGCTGGCTTAACTATGCGGCAT |
| | | CAGAGCAGATTGTACTGAGAGTGCACGATATCGGGTCC |
| 34 | pAAV-hU6(318 bp)- | GTGCACGATATCGGGTCCCCAATTGCTGCGCGCTCGCTCGCTCACTGAGGCCGCCCGGGCAAAGCCC |
| | SGN004282-Jet- | GGGCGTCGGGCGACCTTTGGTCGCCCGGCCTCAGTGAGCGAGCGAGCGCGCAGAGAGGGAGTGGCC |
| | APG05586mco- | AACTCCATCACTAGGGGTTCCTCGTCGACAATGCACGCGTTCGAAGTACTCATGTACAAAAAAGCAG |
| | SV40pA(179 bp) | GCTTTAAAGGAACCAATTCAGTCGACTGGATCCGGTACCAAGGTCGGGCAGGAAGAGGGCCTATTTC |
| | | CCATGATTCCTTCATATTTGCATATACGATACAAGGCTGTTAGAGAGATAATTAGAATTAATTTGACT |
| | | GTAAACACAAAGATATTAGTACAAAATACGTGACGTAGAAAGTAATAATTTCTTGGGTAGTTTGCAG |
| | | TTTTAAAATTATGTTTTAAAATGGACTATCATATGCTTACCGTAACTTGAAAGTATTTCGATTTCTTGG |
| | | CTTTATATATCTTGTGGAAAGGACGAAACACCGACCGGCCTCCAGGATGAAGTGCACAGTTATTGTA |
| | | CTCAAAGGAATCTACAATAATAAGGCATCTTGCCGAATTTACCGCCCTACATATGTAGGGCGGTTTTT |
| | | TTTTGTCTAGACAAGTTGGAGCTCGAATTCGGGCGGAGTTAGGGCGGAGCCAATCAGCGTGCGCCGT |
| | | TCCGAAAGTTGCCTTTTATGGCTGGGCGGAGAATGGGCGGTGAACGCCGATGATTATATAAGGACGC |
| | | GCCGGGTGTGGCACAGCTAGTTCCGTCGCAGCCGGGATTTGGGTCGCGGTTCTTGTTTGTGGATCCCT |
| | | GTGATCGTCACTTGACAGCGGCCGCGCCACCATGGCCCCTAAGAAGAAAAGAAAGGTGATGTACTCT |
| | | ATCGGCCTCGATCTGGGCATCTCTAGCGTCGGCTGGTCGGTGATCGACGAAGAGACAGGCAAAATCG |
| | | TTGACCTGGGCGTGCGGCTGTTTTCTGCTAAGAACAGCGAGAAGAATCTGGAGAGAAGAACCAGCA |
| | | GAGGCGCAAGAAGACTGATCAGAAGAAAGACGAACCGACTAAAAGACGCCAAGAAATTACTGGAG |
| | | GCCATCGGCTTCTACGAAGATAAGGCCCTTAAAAATGTCTGCCCTTACCAACTGAGAGTGAAGGGCC |
| | | TGACCGAGGGCCTGACAAAGGGCGAACTGTACAAGGTCGTGCTGCACATCGTGAAGAAACGGGGCA |
| | | TCAGCTACCTGGACGAGGACGACGCCGAAGCTGCCAAAGAATCTCAGGACTACAAGGAGCAGGTAC |
| | | GCAAAAATGCCCAACTGCTGACAAAGTACACCCCTGGACAGATCCAACTGCAGAGACTGAAGGAGA |
| | | ACAACAGAGTGAAGACAGGAATCAACGGCCAGGGCCACTACCAACTGAACGTGTTCAAGGTGAGCG |
| | | CTTACGCTGACGAGCTGGCCACCATCCTGAAGACCCAGCAAGCCCTGTATCCTAACGAACTGACCGA |
| | | CGACTGGATCGCCCTGTTCGTGCAACCTGGAATTGCCGAGAATGCCGGCCTTATCTACAGAAAGAGG |
| | | CCTTACTACCACGGCCCTGGCAACGAGGCCAACAACAGCCCTTACGGAAGATGGTCCGACTTCCAGA |
| | | AAACAGGCCAGCCAGCCGCTAACATCTTCGACAAGCTAATCGGAAAGGATTTCCAGGGCGAGCTGC |
| | | GGGCCAGCGGCCTGAGCCTGTCTGCCCAGCAATACAACCTGCTGAACGACCTGACCAATCTGAAAAT |
| | | TGACGGAGAGGTGTCCCTGTCCCCCGAGCAGAAGGAATTCATCCTGACCGAGCTGATGACCAAGGA |
| | | ATTCGCCAGATTCGGCGTCAACGACATTGCCAAGCTGCTGGGCGTGAAAAAAGAACAGTTGTCTGGC |
| | | TGGCGGCTGGACAAGAAGGGCAAGCCCGAAATCCACACCCTGAAGGGCTACAGAAACTGGAGAAA |
| | | GATCTTTGCCGAGGCCGGAATCGACCTGGCCACACTGCCAACAGAGACCATTGACTGCCTGGCCAAG |
| | | GTTCTGACCCTGAACACCGAGCGGGAAGGAGTGGAAAACACCCTGGCCTTTGAGCTGCCTGAACTGG |
| | | CTGAGCCTGTGAAGAGCCTGGTGTTGGACCATTACAAGGAACTGAGCCAAAGCATCTCAACACAGG |
| | | CTTGGCACAGATTCAGCCTGAAGACACTGCACCTGCTGATCCCCGAGCTGATCAAGAGCACCAGCGA |
| | | GCAGAACACCCTGCTGGAACAGTTCCAACTGAAGGCCGGCGTGCGCAAGCGGTACTCCGACTACAA |
| | | GAAGCTGCCTACCAAGGAGGTGCTGGCCGAGATCTACAATCCTACTGTGAACAAAACCGTCAGCCA |
| | | GGCCTTCAAGGTGATGGACGCTCTGCTGGAGAAGTACGGCAAGGACCAGATCCACTACATCACCGTG |
| | | GAAATGCCTAGAGATGATAACGAGGAGGAAGAGCGTAAAAGAATTAAGGAACTGCAAACGAAGAA |
| | | TTCTCAGAGAAAGAACGATAGCCAGCAATACTTCCTGCAGAAATCCGGCTGGAGCCAGGAGAAGTT |
| | | CCAGGCCACAATCCACAAGAATAGAAGATTCCTGGCCAAGCTGCTGTACTATTTTGAGCAGGACGGC |
| | | GTGTGTGCCTACACCGGGAACCCCATCAGCCCTGAGCTTCTGGTGTCCGACAGCACCGAGATCGACC |
| | | ACATCATCCCTATCAGCATCAGCCTGGATGACAGCATCAACAACAAAGTGCTGGTGCTGTCTCACGC |
| | | CAACCAGGTGAAGGGCCAACAGACCCCTTACGATGCCAGAATGGCCGGCGCCTTCAACAAGATCAA |
| | | TGGAAAGTTCAGCAACTGGGACGAGTATCAAAAGTGGGTGGAAAGCAGACCTTTCAGCCGGAAAAA |
| | | GGTGAACAACCTCCTGGAGACCAGAAACATTTTCGACAGCGAGCAGGTGCAAAAGTTTCTGAGCAG |
| | | AAACCTGAATGATACAAGATACGCCAGCAGACTGGTCCTGAATACCCTGCAGAGCTTTTTCGAGAAC |
| | | CAGGATACCATTGTGCGGGTGGTCAACGGCAGCTTCACCCATACCCTGAGAAAGAAGTGGGGCGCC |
| | | GATCTGGACAAAACCCGGGAGACACACCACCACCACGCCGTGGACGCCACACTGTGCGCGGTGACA |
| | | CCCTTCGTGAAGGTCTCTAGATACCACTACGCCGTGAACGAGGAGACCGGCGAGAAATTCATGAGA |
| | | GAAATCGATGTGGAGACCGGCGAAATCCTGGACGAAATTCCATACAGAGAGTACAAGAAGGCCAAG |
| | | CACTACGAGAGAAAAACCTACCAGGTGAAGTGGTCCAACTTCCGCGAGCAGCTTAAGCCCATCACA |
| | | ATCCATCCTAAGATCAAGTTCAGCCACCAGGTTGACAGAAAAGCCAACAGAAAGCTGAGCGACGCT |
| | | ACCATCTACTCCGTTAGAGAGAAGACAGAGGTGAAAACACTGAAGAGCGGCAAGGAGAAGATCACG |
| | | ACCGACGAATACACCATCGGCAAGATCAAGGACATCTATACCGTAGACGGCTGGGAAGCCTTTAAA |
| | | AAGAAGCAGGACAAACTGCTCATGAAGGAATTCGATGAAAAGACCTACGAGCTGCTGGTGACCATC |
| | | GCCGCCACCACCCCTGATTTTCAGGAGGTGGAAGAAAAGAACGGCAAAGTGAAGAGAGTGAAGCGG |
| | | AGCCCTTTCGCCGTGTACTGTGAGGAAAACGGCATACCCGCCATCCGGAAATACGCCAAGAAGAAC |
| | | AACGGCCCCGTGATCCGGAGCCTGAAGTACTACGACGGCAAGCTGAATAAGCACATCAACATCACC |
| | | AAGGATGAGAAGGGCAGGCCCGTGGAGCAGACAAAGAACGGCCGGAAAGTGACTCTGCAGAGCCT |
| | | GAAACCCTACCGGTATGATATCTACCAGGACCTCGAAACCAAGGCCTATTATACAGTGCAACTGTAC |
| | | TACAGCGACCTGCGCTTTGTGGAGGGCGAGTACGGAATCACAGAAAAAGAGTACATGAAGAAGGTG |
| | | GCCGAACAGACCAAAGGACAGGTGGTGCGGTTCTGCTTCAGCCTGCAGAAGAACGACGGCCTCGAA |
| | | ATCGAGTGGAAGGATTCTCAACGGTACGACGTGAGATTCTACAATTTCCAATCTGCTAACAGCATCA |
| | | ACTTCAAGGGCTTAGAACAGGAGATGATCCCTGCTGAAAACCAGTTCAAGCAGAAGCCTTACAACA |
| | | ATGGCGCCATCAACCTGAACATCGCCAAGTATGGCAAGGAGGGCAAGAAACTGAGAAAGTTCAACA |
| | | CCGATATCCTGGGTAAAAAGCACCACCTGTCTTATGAGAAAGAGCCGAAGAACATCATCAAGAGCG |
| | | GCGGAAGCAAAAGACCTGCCGCTACAAAGAAGGCCGGCCAGGCCAAGAAAAAGAAGTAAGCTAGC |
| | | TTGACTGACTGAGATACAGCGTACCTTCAGCTCACAGACATGATAAGATACATTGATGAGTTTGGAC |
| | | AAACCACAACTAGAATGCAGTGAAAAAAATGCTTTATTTGTGAAATTTGTGATGCTATTGCTTTATTT |
| | | GTAACCATTATAAGCTGCAATAAACAAGTTAACAACAACAATTGCATTCATTTTATGTTTCAGGTTCA |
| | | GGGGGAGGTGTGGGAGGTTTTTTAAAGCAAGTAAAAGCTTGGTAGGAACCCCTAGTGATGGAGTTG |
| | | GCCACTCCCTCTCTGCGCGCTCGCTCGCTCACTGAGGCCGGGCGACCAAAGGTCGCCCGACGCCCGG |
| | | GCTTTGCCCGGGCGGCCTCAGTGAGCGAGCGAGCGCGCAGAGGCATTTAATTAAGCAAGCTGTCATA |
| | | GCTGTTTCCTGTGTGAAATTGTTATCCGCTCACAATTCCACACAACATACGAGCCGGAAGCATAAAG |
| | | TGTAAAGCCTGGGGTGCCTAATGAGTGAGCTAACTCACATTAATTGCGTTGCGCTCACTGCCCGCTTT |
| | | CCAGTCGGGAAACCTGTCGTGCCAGCTGCATTAATGAATCGGCCAACGCGCGGGGAGAGGCGGTTT |
| | | GCGTATTGGGCGCTCTTCCGCTTCCTCGCTCACTGACTCGCTGCGCTCGGTCGTTCGGCTGCGGCGAG |
| | | CGGTATCAGCTCACTCAAAGGCGGTAATACGGTTATCCACAGAATCAGGGGATAACGCAGGAAAGA |
| | | ACATGTGAGCAAAAGGCCAGCAAAAGGCCAGGAACCGTAAAAAGGCCGCGTTGCTGGCGTTTTTCC |
| | | ATAGGCTCCGCCCCCCTGACGAGCATCACAAAAATCGACGCTCAAGTCAGAGGTGGCGAAACCCGA |
| | | CAGGACTATAAAGATACCAGGCGTTTCCCCCTGGAAGCTCCCTCGTGCGCTCTCCTGTTCCGACCCTG |
| | | CCGCTTACCGGATACCTGTCCGCCTTTCTCCCTTCGGGAAGCGTGGCGCTTTCTCATAGCTCACGCTG |
| | | TAGGTATCTCAGTTCGGTGTAGGTCGTTCGCTCCAAGCTGGGCTGTGTGCACGAACCCCCCGTTCAGC |
| | | CCGACCGCTGCGCCTTATCCGGTAACTATCGTCTTGAGTCCAACCCGGTAAGACACGACTTATCGCC |
| | | ACTGGCAGCAGCCACTGGTAACAGGATTAGCAGAGCGAGGTATGTAGGCGGTGCTACAGAGTTCTT |
| | | GAAGTGGTGGCCTAACTACGGCTACACTAGAAGAACAGTATTTGGTATCTGCGCTCTGCTGAAGCCA |
| | | GTTACCTTCGGAAAAAGAGTTGGTAGCTCTTGATCCGGCAAACAAACCACCGCTGGTAGCGGTGGTT |
| | | TTTTTGTTTGCAAGCAGCAGATTACGCGCAGAAAAAAAGGATCTCAAGAAGATCCTTTGATCTTTTCT |
| | | ACGGGGTCTGACGCTCAGTGGAACGAAAACTCACGTTAAGGGATTTTGGTCATGAGATTATCAAAAA |
| | | GGATCTTCACCTAGATCCTTTTAAATTAAAAATGAAGTTTTAAATCAAGCCCAATCTGAATAATGTTA |
| | | CAACCAATTAACCAATTCTGATTAGAAAAACTCATCGAGCATCAAATGAAACTGCAATTTATTCATA |
| | | TCAGGATTATCAATACCATATTTTTGAAAAAGCCGTTTCTGTAATGAAGGAGAAAACTCACCGAGGC |
| | | AGTTCCATAGGATGGCAAGATCCTGGTATCGGTCTGCGATTCCGACTCGTCCAACATCAATACAACC |
| | | TATTAATTTCCCCTCGTCAAAAATAAGGTTATCAAGTGAGAAATCACCATGAGTGACGACTGAATCC |
| | | GGTGAGAATGGCAAAAGTTTATGCATTTCTTTCCAGACTTGTTCAACAGGCCAGCCATTACGCTCGTC |
| | | ATCAAAATCACTCGCATCAACCAAACCGTTATTCATTCGTGATTGCGCCTGAGCGAGACGAAATACG |
| | | CGATCGCTGTTAAAAGGACAATTACAAACAGGAATCGAATGCAACCGGCGCAGGAACACTGCCAGC |
| | | GCATCAACAATATTTTCACCTGAATCAGGATATTCTTCTAATACCTGGAATGCTGTTTTTCCGGGGAT |
| | | CGCAGTGGTGAGTAACCATGCATCATCAGGAGTACGGATAAAATGCTTGATGGTCGGAAGAGGCAT |
| | | AAATTCCGTCAGCCAGTTTAGTCTGACCATCTCATCTGTAACATCATTGGCAACGCTACCTTTGCCAT |
| | | GTTTCAGAAACAACTCTGGCGCATCGGGCTTCCCATACAAGCGATAGATTGTCGCACCTGATTGCCC |
| | | GACATTATCGCGAGCCCATTTATACCCATATAAATCAGCATCCATGTTGGAATTTAATCGCGGCCTCG |
| | | ACGTTTCCCGTTGAATATGGCTCATAACACCCCTTGTATTACTGTTTATGTAAGCAGACAGTTTTATT |
| | | GTTCATGATGATATATTTTTATCTTGTGCAATGTAACATCAGAGATTTTGAGACACGGGCCAGAGCTG |
| | | CATCGCGCGTTTCGGTGATGACGGTGAAAACCTCTGACACATGCAGCTCCCGGAGACGGTCACAGCT |
| | | TGTCTGTAAGCGGATGCCGGGAGCAGACAAGCCCGTCAGGGCGCGTCAGCGGGTGTTGGCGGGTGT |
| | | CGGGGCTGGCTTAACTATGCGGCATCAGAGCAGATTGTACTGAGAGTGCACGATATCGGGTCC |
| 35 | pAAV-hU6(318 bp)- | GTGCACGATATCGGGTCCCCAATTGCTGCGCGCTCGCTCGCTCACTGAGGCCGCCCGGGCAAAGCCC |
| | SGN004282-hSyn- | GGGCGTCGGGCGACCTTTGGTCGCCCGGCCTCAGTGAGCGAGCGAGCGCGCAGAGAGGGAGTGGCC |
| | APG05586mco- | AACTCCATCACTAGGGGTTCCTCGTCGACAATGCACGCGTTCGAAGTACTCATGTACAAAAAAGCAG |
| | SV40pA(179 bp) | GCTTTAAAGGAACCAATTCAGTCGACTGGATCCGGTACCAAGGTCGGGCAGGAAGAGGGCCTATTTC |
| | | CCATGATTCCTTCATATTTGCATATACGATACAAGGCTGTTAGAGAGATAATTAGAATTAATTTGACT |
| | | GTAAACACAAAGATATTAGTACAAAATACGTGACGTAGAAAGTAATAATTTCTTGGGTAGTTTGCAG |
| | | TTTTAAAATTATGTTTTAAAATGGACTATCATATGCTTACCGTAACTTGAAAGTATTTCGATTTCTTGG |
| | | CTTTATATATCTTGTGGAAAGGACGAAACACCGACCGGCCTCCAGGATGAAGTGCACAGTTATTGTA |
| | | CTCAAAGGAATCTACAATAATAAGGCATCTTGCCGAATTTACCGCCCTACATATGTAGGGCGGTTTTT |
| | | TTTTGTCTAGACAAGTTGGAGCTCAGTGCAAGTGGGTTTTAGGACCAGGATGAGGCGGGGTGGGGGT |
| | | GCCTACCTGACGACCGACCCCGACCCACTGGACAAGCACCCAACCCCCATTCCCCAAATTGCGCATC |
| | | CCCTATCAGAGAGGGGGAGGGGAAACAGGATGCGGCGAGGCGCGTGCGCACTGCCAGCTTCAGCAC |
| | | CGCGGACAGTGCCTTCGCCCCCGCCTGGCGGCGCGCGCCACCGCCGCCTCAGCACTGAAGGCGCGCT |
| | | GACGTCACTCGCCGGTCCCCCGCAAACTCCCCTTCCCGGCCACCTTGGTCGCGTCCGCGCCGCCGCCG |
| | | GCCCAGCCGGACCGCACCACGCGAGGCGCGAGATAGGGGGGCACGGGCGCGACCATCTGCGCTGCG |
| | | GCGCCGGCGACTCAGCGCTGCCTCAGTCTGCGGTGGGCAGCGGAGGAGTCGTGTCGTGCCTGAGAGC |
| | | GCAGTCGAGAAGGTACCGGATCCCCGGGTACCGGTCGCCACCATGGCCCCTAAGAAGAAAAGAAAG |
| | | GTGATGTACTCTATCGGCCTCGATCTGGGCATCTCTAGCGTCGGCTGGTCGGTGATCGACGAAGAGA |
| | | CAGGCAAAATCGTTGACCTGGGCGTGCGGCTGTTTTCTGCTAAGAACAGCGAGAAGAATCTGGAGA |
| | | GAAGAACCAGCAGAGGCGCAAGAAGACTGATCAGAAGAAAGACGAACCGACTAAAAGACGCCAAG |
| | | AAATTACTGGAGGCCATCGGCTTCTACGAAGATAAGGCCCTTAAAAATGTCTGCCCTTACCAACTGA |
| | | GAGTGAAGGGCCTGACCGAGGGCCTGACAAAGGGCGAACTGTACAAGGTCGTGCTGCACATCGTGA |
| | | AGAAACGGGGCATCAGCTACCTGGACGAGGACGACGCCGAAGCTGCCAAAGAATCTCAGGACTACA |
| | | AGGAGCAGGTACGCAAAAATGCCCAACTGCTGACAAAGTACACCCCTGGACAGATCCAACTGCAGA |
| | | GACTGAAGGAGAACAACAGAGTGAAGACAGGAATCAACGGCCAGGGCCACTACCAACTGAACGTGT |
| | | TCAAGGTGAGCGCTTACGCTGACGAGCTGGCCACCATCCTGAAGACCCAGCAAGCCCTGTATCCTAA |
| | | CGAACTGACCGACGACTGGATCGCCCTGTTCGTGCAACCTGGAATTGCCGAGAATGCCGGCCTTATC |
| | | TACAGAAAGAGGCCTTACTACCACGGCCCTGGCAACGAGGCCAACAACAGCCCTTACGGAAGATGG |
| | | TCCGACTTCCAGAAAACAGGCCAGCCAGCCGCTAACATCTTCGACAAGCTAATCGGAAAGGATTTCC |
| | | AGGGCGAGCTGCGGGCCAGCGGCCTGAGCCTGTCTGCCCAGCAATACAACCTGCTGAACGACCTGA |
| | | CCAATCTGAAAATTGACGGAGAGGTGTCCCTGTCCCCCGAGCAGAAGGAATTCATCCTGACCGAGCT |
| | | GATGACCAAGGAATTCGCCAGATTCGGCGTCAACGACATTGCCAAGCTGCTGGGCGTGAAAAAAGA |
| | | ACAGTTGTCTGGCTGGCGGCTGGACAAGAAGGGCAAGCCCGAAATCCACACCCTGAAGGGCTACAG |
| | | AAACTGGAGAAAGATCTTTGCCGAGGCCGGAATCGACCTGGCCACACTGCCAACAGAGACCATTGA |
| | | CTGCCTGGCCAAGGTTCTGACCCTGAACACCGAGCGGGAAGGAGTGGAAAACACCCTGGCCTTTGA |
| | | GCTGCCTGAACTGGCTGAGCCTGTGAAGAGCCTGGTGTTGGACCATTACAAGGAACTGAGCCAAAGC |
| | | ATCTCAACACAGGCTTGGCACAGATTCAGCCTGAAGACACTGCACCTGCTGATCCCCGAGCTGATCA |
| | | AGAGCACCAGCGAGCAGAACACCCTGCTGGAACAGTTCCAACTGAAGGCCGGCGTGCGCAAGCGGT |
| | | ACTCCGACTACAAGAAGCTGCCTACCAAGGAGGTGCTGGCCGAGATCTACAATCCTACTGTGAACAA |
| | | AACCGTCAGCCAGGCCTTCAAGGTGATGGACGCTCTGCTGGAGAAGTACGGCAAGGACCAGATCCA |
| | | CTACATCACCGTGGAAATGCCTAGAGATGATAACGAGGAGGAAGAGCGTAAAAGAATTAAGGAACT |
| | | GCAAACGAAGAATTCTCAGAGAAAGAACGATAGCCAGCAATACTTCCTGCAGAAATCCGGCTGGAG |
| | | CCAGGAGAAGTTCCAGGCCACAATCCACAAGAATAGAAGATTCCTGGCCAAGCTGCTGTACTATTTT |
| | | GAGCAGGACGGCGTGTGTGCCTACACCGGGAACCCCATCAGCCCTGAGCTTCTGGTGTCCGACAGCA |
| | | CCGAGATCGACCACATCATCCCTATCAGCATCAGCCTGGATGACAGCATCAACAACAAAGTGCTGGT |
| | | GCTGTCTCACGCCAACCAGGTGAAGGGCCAACAGACCCCTTACGATGCCAGAATGGCCGGCGCCTTC |
| | | AACAAGATCAATGGAAAGTTCAGCAACTGGGACGAGTATCAAAAGTGGGTGGAAAGCAGACCTTTC |
| | | AGCCGGAAAAAGGTGAACAACCTCCTGGAGACCAGAAACATTTTCGACAGCGAGCAGGTGCAAAAG |
| | | TTTCTGAGCAGAAACCTGAATGATACAAGATACGCCAGCAGACTGGTCCTGAATACCCTGCAGAGCT |
| | | TTTTCGAGAACCAGGATACCATTGTGCGGGTGGTCAACGGCAGCTTCACCCATACCCTGAGAAAGAA |
| | | GTGGGGCGCCGATCTGGACAAAACCCGGGAGACACACCACCACCACGCCGTGGACGCCACACTGTG |
| | | CGCGGTGACACCCTTCGTGAAGGTCTCTAGATACCACTACGCCGTGAACGAGGAGACCGGCGAGAA |
| | | ATTCATGAGAGAAATCGATGTGGAGACCGGCGAAATCCTGGACGAAATTCCATACAGAGAGTACAA |
| | | GAAGGCCAAGCACTACGAGAGAAAAACCTACCAGGTGAAGTGGTCCAACTTCCGCGAGCAGCTTAA |
| | | GCCCATCACAATCCATCCTAAGATCAAGTTCAGCCACCAGGTTGACAGAAAAGCCAACAGAAAGCT |
| | | GAGCGACGCTACCATCTACTCCGTTAGAGAGAAGACAGAGGTGAAAACACTGAAGAGCGGCAAGGA |
| | | GAAGATCACGACCGACGAATACACCATCGGCAAGATCAAGGACATCTATACCGTAGACGGCTGGGA |
| | | AGCCTTTAAAAAGAAGCAGGACAAACTGCTCATGAAGGAATTCGATGAAAAGACCTACGAGCTGCT |
| | | GGTGACCATCGCCGCCACCACCCCTGATTTTCAGGAGGTGGAAGAAAAGAACGGCAAAGTGAAGAG |
| | | AGTGAAGCGGAGCCCTTTCGCCGTGTACTGTGAGGAAAACGGCATACCCGCCATCCGGAAATACGCC |
| | | AAGAAGAACAACGGCCCCGTGATCCGGAGCCTGAAGTACTACGACGGCAAGCTGAATAAGCACATC |
| | | AACATCACCAAGGATGAGAAGGGCAGGCCCGTGGAGCAGACAAAGAACGGCCGGAAAGTGACTCT |
| | | GCAGAGCCTGAAACCCTACCGGTATGATATCTACCAGGACCTCGAAACCAAGGCCTATTATACAGTG |
| | | CAACTGTACTACAGCGACCTGCGCTTTGTGGAGGGCGAGTACGGAATCACAGAAAAAGAGTACATG |
| | | AAGAAGGTGGCCGAACAGACCAAAGGACAGGTGGTGCGGTTCTGCTTCAGCCTGCAGAAGAACGAC |
| | | GGCCTCGAAATCGAGTGGAAGGATTCTCAACGGTACGACGTGAGATTCTACAATTTCCAATCTGCTA |
| | | ACAGCATCAACTTCAAGGGCTTAGAACAGGAGATGATCCCTGCTGAAAACCAGTTCAAGCAGAAGC |
| | | CTTACAACAATGGCGCCATCAACCTGAACATCGCCAAGTATGGCAAGGAGGGCAAGAAACTGAGAA |
| | | AGTTCAACACCGATATCCTGGGTAAAAAGCACCACCTGTCTTATGAGAAAGAGCCGAAGAACATCAT |
| | | CAAGAGCGGCGGAAGCAAAAGACCTGCCGCTACAAAGAAGGCCGGCCAGGCCAAGAAAAAGAAGT |
| | | AAGCTAGCTTGACTGACTGAGATACAGCGTACCTTCAGCTCACAGACATGATAAGATACATTGATGA |
| | | GTTTGGACAAACCACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCATTCATTTTATGTT |
| | | TCAGGTTCAGGGGGAGGTGTGGGAGGTTTTTTAAAGCAAGTAAAAGCTTGGTAGGAACCCCTAGTGA |
| | | TGGAGTTGGCCACTCCCTCTCTGCGCGCTCGCTCGCTCACTGAGGCCGGGCGACCAAAGGTCGCCCG |
| | | ACGCCCGGGCTTTGCCCGGGCGGCCTCAGTGAGCGAGCGAGCGCGCAGAGGCATTTAATTAAGCAA |
| | | GCTGTCATAGCTGTTTCCTGTGTGAAATTGTTATCCGCTCACAATTCCACACAACATACGAGCCGGAA |
| | | GCATAAAGTGTAAAGCCTGGGGTGCCTAATGAGTGAGCTAACTCACATTAATTGCGTTGCGCTCACT |
| | | GCCCGCTTTCCAGTCGGGAAACCTGTCGTGCCAGCTGCATTAATGAATCGGCCAACGCGCGGGGAGA |
| | | GGCGGTTTGCGTATTGGGCGCTCTTCCGCTTCCTCGCTCACTGACTCGCTGCGCTCGGTCGTTCGGCT |
| | | GCGGCGAGCGGTATCAGCTCACTCAAAGGCGGTAATACGGTTATCCACAGAATCAGGGGATAACGC |
| | | AGGAAAGAACATGTGAGCAAAAGGCCAGCAAAAGGCCAGGAACCGTAAAAAGGCCGCGTTGCTGG |
| | | CGTTTTTCCATAGGCTCCGCCCCCCTGACGAGCATCACAAAAATCGACGCTCAAGTCAGAGGTGGCG |
| | | AAACCCGACAGGACTATAAAGATACCAGGCGTTTCCCCCTGGAAGCTCCCTCGTGCGCTCTCCTGTT |
| | | CCGACCCTGCCGCTTACCGGATACCTGTCCGCCTTTCTCCCTTCGGGAAGCGTGGCGCTTTCTCATAG |
| | | CTCACGCTGTAGGTATCTCAGTTCGGTGTAGGTCGTTCGCTCCAAGCTGGGCTGTGTGCACGAACCCC |
| | | CCGTTCAGCCCGACCGCTGCGCCTTATCCGGTAACTATCGTCTTGAGTCCAACCCGGTAAGACACGA |
| | | CTTATCGCCACTGGCAGCAGCCACTGGTAACAGGATTAGCAGAGCGAGGTATGTAGGCGGTGCTACA |
| | | GAGTTCTTGAAGTGGTGGCCTAACTACGGCTACACTAGAAGAACAGTATTTGGTATCTGCGCTCTGC |
| | | TGAAGCCAGTTACCTTCGGAAAAAGAGTTGGTAGCTCTTGATCCGGCAAACAAACCACCGCTGGTAG |
| | | CGGTGGTTTTTTTGTTTGCAAGCAGCAGATTACGCGCAGAAAAAAAGGATCTCAAGAAGATCCTTTG |
| | | ATCTTTTCTACGGGGTCTGACGCTCAGTGGAACGAAAACTCACGTTAAGGGATTTTGGTCATGAGAT |
| | | TATCAAAAAGGATCTTCACCTAGATCCTTTTAAATTAAAAATGAAGTTTTAAATCAAGCCCAATCTG |
| | | AATAATGTTACAACCAATTAACCAATTCTGATTAGAAAAACTCATCGAGCATCAAATGAAACTGCAA |
| | | TTTATTCATATCAGGATTATCAATACCATATTTTTGAAAAAGCCGTTTCTGTAATGAAGGAGAAAACT |
| | | CACCGAGGCAGTTCCATAGGATGGCAAGATCCTGGTATCGGTCTGCGATTCCGACTCGTCCAACATC |
| | | AATACAACCTATTAATTTCCCCTCGTCAAAAATAAGGTTATCAAGTGAGAAATCACCATGAGTGACG |
| | | ACTGAATCCGGTGAGAATGGCAAAAGTTTATGCATTTCTTTCCAGACTTGTTCAACAGGCCAGCCATT |
| | | ACGCTCGTCATCAAAATCACTCGCATCAACCAAACCGTTATTCATTCGTGATTGCGCCTGAGCGAGA |
| | | CGAAATACGCGATCGCTGTTAAAAGGACAATTACAAACAGGAATCGAATGCAACCGGCGCAGGAAC |
| | | ACTGCCAGCGCATCAACAATATTTTCACCTGAATCAGGATATTCTTCTAATACCTGGAATGCTGTTTT |
| | | TCCGGGGATCGCAGTGGTGAGTAACCATGCATCATCAGGAGTACGGATAAAATGCTTGATGGTCGGA |
| | | AGAGGCATAAATTCCGTCAGCCAGTTTAGTCTGACCATCTCATCTGTAACATCATTGGCAACGCTACC |
| | | TTTGCCATGTTTCAGAAACAACTCTGGCGCATCGGGCTTCCCATACAAGCGATAGATTGTCGCACCTG |
| | | ATTGCCCGACATTATCGCGAGCCCATTTATACCCATATAAATCAGCATCCATGTTGGAATTTAATCGC |
| | | GGCCTCGACGTTTCCCGTTGAATATGGCTCATAACACCCCTTGTATTACTGTTTATGTAAGCAGACAG |
| | | TTTTATTGTTCATGATGATATATTTTTATCTTGTGCAATGTAACATCAGAGATTTTGAGACACGGGCC |
| | | AGAGCTGCATCGCGCGTTTCGGTGATGACGGTGAAAACCTCTGACACATGCAGCTCCCGGAGACGGT |
| | | CACAGCTTGTCTGTAAGCGGATGCCGGGAGCAGACAAGCCCGTCAGGGCGCGTCAGCGGGTGTTGG |
| | | CGGGTGTCGGGGCTGGCTTAACTATGCGGCATCAGAGCAGATTGTACTGAGAGTGCACGATATCGGG |
| | | TCC |
| 36 | pAAV-hU6(318 bp)- | GTGCACGATATCGGGTCCCCAATTGCTGCGCGCTCGCTCGCTCACTGAGGCCGCCCGGGCAAAGCCC |
| | SGN004282- | GGGCGTCGGGCGACCTTTGGTCGCCCGGCCTCAGTGAGCGAGCGAGCGCGCAGAGAGGGAGTGGCC |
| | CMVeb- | AACTCCATCACTAGGGGTTCCTCGTCGACAATGCACGCGTTCGAAGTACTCATGTACAAAAAAGCAG |
| | APG05586mco- | GCTTTAAAGGAACCAATTCAGTCGACTGGATCCGGTACCAAGGTCGGGCAGGAAGAGGGCCTATTTC |
| | SV40pA(179 bp) | CCATGATTCCTTCATATTTGCATATACGATACAAGGCTGTTAGAGAGATAATTAGAATTAATTTGACT |
| | | GTAAACACAAAGATATTAGTACAAAATACGTGACGTAGAAAGTAATAATTTCTTGGGTAGTTTGCAG |
| | | TTTTAAAATTATGTTTTAAAATGGACTATCATATGCTTACCGTAACTTGAAAGTATTTCGATTTCTTGG |
| | | CTTTATATATCTTGTGGAAAGGACGAAACACCGACCGGCCTCCAGGATGAAGTGCACAGTTATTGTA |
| | | CTCAAAGGAATCTACAATAATAAGGCATCTTGCCGAATTTACCGCCCTACATATGTAGGGCGGTTTTT |
| | | TTTTGTCTAGACAAGTTGGAGCTCGTGATGCGGTTTTGGCAGTACATCAATGGGCGTGGATAGCGGT |
| | | TTGACTCACGGGGATTTCCAAGTCTCCACCCCATTGACGTCAATGGGAGTTTGTTTTGGCACCAAAAT |
| | | CAACGGGACTTTCCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTCTTTGTCGAGCCACCA |
| | | TGGCCCCTAAGAAGAAAAGAAAGGTGATGTACTCTATCGGCCTCGATCTGGGCATCTCTAGCGTCGG |
| | | CTGGTCGGTGATCGACGAAGAGACAGGCAAAATCGTTGACCTGGGCGTGCGGCTGTTTTCTGCTAAG |
| | | AACAGCGAGAAGAATCTGGAGAGAAGAACCAGCAGAGGCGCAAGAAGACTGATCAGAAGAAAGAC |
| | | GAACCGACTAAAAGACGCCAAGAAATTACTGGAGGCCATCGGCTTCTACGAAGATAAGGCCCTTAA |
| | | AAATGTCTGCCCTTACCAACTGAGAGTGAAGGGCCTGACCGAGGGCCTGACAAAGGGCGAACTGTA |
| | | CAAGGTCGTGCTGCACATCGTGAAGAAACGGGGCATCAGCTACCTGGACGAGGACGACGCCGAAGC |
| | | TGCCAAAGAATCTCAGGACTACAAGGAGCAGGTACGCAAAAATGCCCAACTGCTGACAAAGTACAC |
| | | CCCTGGACAGATCCAACTGCAGAGACTGAAGGAGAACAACAGAGTGAAGACAGGAATCAACGGCC |
| | | AGGGCCACTACCAACTGAACGTGTTCAAGGTGAGCGCTTACGCTGACGAGCTGGCCACCATCCTGAA |
| | | GACCCAGCAAGCCCTGTATCCTAACGAACTGACCGACGACTGGATCGCCCTGTTCGTGCAACCTGGA |
| | | ATTGCCGAGAATGCCGGCCTTATCTACAGAAAGAGGCCTTACTACCACGGCCCTGGCAACGAGGCCA |
| | | ACAACAGCCCTTACGGAAGATGGTCCGACTTCCAGAAAACAGGCCAGCCAGCCGCTAACATCTTCGA |
| | | CAAGCTAATCGGAAAGGATTTCCAGGGCGAGCTGCGGGCCAGCGGCCTGAGCCTGTCTGCCCAGCA |
| | | ATACAACCTGCTGAACGACCTGACCAATCTGAAAATTGACGGAGAGGTGTCCCTGTCCCCCGAGCAG |
| | | AAGGAATTCATCCTGACCGAGCTGATGACCAAGGAATTCGCCAGATTCGGCGTCAACGACATTGCCA |
| | | AGCTGCTGGGCGTGAAAAAAGAACAGTIGTCTGGCTGGCGGCTGGACAAGAAGGGCAAGCCCGAAA |
| | | TCCACACCCTGAAGGGCTACAGAAACTGGAGAAAGATCTTTGCCGAGGCCGGAATCGACCTGGCCA |
| | | CACTGCCAACAGAGACCATTGACTGCCTGGCCAAGGTTCTGACCCTGAACACCGAGCGGGAAGGAG |
| | | TGGAAAACACCCTGGCCTTTGAGCTGCCTGAACTGGCTGAGCCTGTGAAGAGCCTGGTGTTGGACCA |
| | | TTACAAGGAACTGAGCCAAAGCATCTCAACACAGGCTTGGCACAGATTCAGCCTGAAGACACTGCA |
| | | CCTGCTGATCCCCGAGCTGATCAAGAGCACCAGCGAGCAGAACACCCTGCTGGAACAGTTCCAACTG |
| | | AAGGCCGGCGTGCGCAAGCGGTACTCCGACTACAAGAAGCTGCCTACCAAGGAGGTGCTGGCCGAG |
| | | ATCTACAATCCTACTGTGAACAAAACCGTCAGCCAGGCCTTCAAGGTGATGGACGCTCTGCTGGAGA |
| | | AGTACGGCAAGGACCAGATCCACTACATCACCGTGGAAATGCCTAGAGATGATAACGAGGAGGAAG |
| | | AGCGTAAAAGAATTAAGGAACTGCAAACGAAGAATTCTCAGAGAAAGAACGATAGCCAGCAATACT |
| | | TCCTGCAGAAATCCGGCTGGAGCCAGGAGAAGTTCCAGGCCACAATCCACAAGAATAGAAGATTCC |
| | | TGGCCAAGCTGCTGTACTATTTTGAGCAGGACGGCGTGTGTGCCTACACCGGGAACCCCATCAGCCC |
| | | TGAGCTTCTGGTGTCCGACAGCACCGAGATCGACCACATCATCCCTATCAGCATCAGCCTGGATGAC |
| | | AGCATCAACAACAAAGTGCTGGTGCTGTCTCACGCCAACCAGGTGAAGGGCCAACAGACCCCTTAC |
| | | GATGCCAGAATGGCCGGCGCCTTCAACAAGATCAATGGAAAGTTCAGCAACTGGGACGAGTATCAA |
| | | AAGTGGGTGGAAAGCAGACCTTTCAGCCGGAAAAAGGTGAACAACCTCCTGGAGACCAGAAACATT |
| | | TTCGACAGCGAGCAGGTGCAAAAGTTTCTGAGCAGAAACCTGAATGATACAAGATACGCCAGCAGA |
| | | CTGGTCCTGAATACCCTGCAGAGCTTTTTCGAGAACCAGGATACCATTGTGCGGGTGGTCAACGGCA |
| | | GCTTCACCCATACCCTGAGAAAGAAGTGGGGCGCCGATCTGGACAAAACCCGGGAGACACACCACC |
| | | ACCACGCCGTGGACGCCACACTGTGCGCGGTGACACCCTTCGTGAAGGTCTCTAGATACCACTACGC |
| | | CGTGAACGAGGAGACCGGCGAGAAATTCATGAGAGAAATCGATGTGGAGACCGGCGAAATCCTGGA |
| | | CGAAATTCCATACAGAGAGTACAAGAAGGCCAAGCACTACGAGAGAAAAACCTACCAGGTGAAGTG |
| | | GTCCAACTTCCGCGAGCAGCTTAAGCCCATCACAATCCATCCTAAGATCAAGTTCAGCCACCAGGTT |
| | | GACAGAAAAGCCAACAGAAAGCTGAGCGACGCTACCATCTACTCCGTTAGAGAGAAGACAGAGGTG |
| | | AAAACACTGAAGAGCGGCAAGGAGAAGATCACGACCGACGAATACACCATCGGCAAGATCAAGGA |
| | | CATCTATACCGTAGACGGCTGGGAAGCCTTTAAAAAGAAGCAGGACAAACTGCTCATGAAGGAATT |
| | | CGATGAAAAGACCTACGAGCTGCTGGTGACCATCGCCGCCACCACCCCTGATTTTCAGGAGGTGGAA |
| | | GAAAAGAACGGCAAAGTGAAGAGAGTGAAGCGGAGCCCTTTCGCCGTGTACTGTGAGGAAAACGGC |
| | | ATACCCGCCATCCGGAAATACGCCAAGAAGAACAACGGCCCCGTGATCCGGAGCCTGAAGTACTAC |
| | | GACGGCAAGCTGAATAAGCACATCAACATCACCAAGGATGAGAAGGGCAGGCCCGTGGAGCAGAC |
| | | AAAGAACGGCCGGAAAGTGACTCTGCAGAGCCTGAAACCCTACCGGTATGATATCTACCAGGACCT |
| | | CGAAACCAAGGCCTATTATACAGTGCAACTGTACTACAGCGACCTGCGCTTTGTGGAGGGCGAGTAC |
| | | GGAATCACAGAAAAAGAGTACATGAAGAAGGTGGCCGAACAGACCAAAGGACAGGTGGTGCGGTT |
| | | CTGCTTCAGCCTGCAGAAGAACGACGGCCTCGAAATCGAGTGGAAGGATTCTCAACGGTACGACGT |
| | | GAGATTCTACAATTTCCAATCTGCTAACAGCATCAACTTCAAGGGCTTAGAACAGGAGATGATCCCT |
| | | GCTGAAAACCAGTTCAAGCAGAAGCCTTACAACAATGGCGCCATCAACCTGAACATCGCCAAGTAT |
| | | GGCAAGGAGGGCAAGAAACTGAGAAAGTTCAACACCGATATCCTGGGTAAAAAGCACCACCTGTCT |
| | | TATGAGAAAGAGCCGAAGAACATCATCAAGAGCGGCGGAAGCAAAAGACCTGCCGCTACAAAGAA |
| | | GGCCGGCCAGGCCAAGAAAAAGAAGTAAGCTAGCTTGACTGACTGAGATACAGCGTACCTTCAGCT |
| | | CACAGACATGATAAGATACATTGATGAGTTTGGACAAACCACAACTAGAATGCAGTGAAAAAAATG |
| | | CTTTATTTGTGAAATTTGTGATGCTATTGCTTTATTTGTAACCATTATAAGCTGCAATAAACAAGTTA |
| | | ACAACAACAATTGCATTCATTTTATGTTTCAGGTTCAGGGGGAGGTGTGGGAGGTTTTTTAAAGCAA |
| | | GTAAAAGCTTGGTAGGAACCCCTAGTGATGGAGTTGGCCACTCCCTCTCTGCGCGCTCGCTCGCTCA |
| | | CTGAGGCCGGGCGACCAAAGGTCGCCCGACGCCCGGGCTTTGCCCGGGCGGCCTCAGTGAGCGAGC |
| | | GAGCGCGCAGAGGCATTTAATTAAGCAAGCTGTCATAGCTGTTTCCTGTGTGAAATTGTTATCCGCTC |
| | | ACAATTCCACACAACATACGAGCCGGAAGCATAAAGTGTAAAGCCTGGGGTGCCTAATGAGTGAGC |
| | | TAACTCACATTAATTGCGTTGCGCTCACTGCCCGCTTTCCAGTCGGGAAACCTGTCGTGCCAGCTGCA |
| | | TTAATGAATCGGCCAACGCGCGGGGAGAGGCGGTTTGCGTATTGGGCGCTCTTCCGCTTCCTCGCTC |
| | | ACTGACTCGCTGCGCTCGGTCGTTCGGCTGCGGCGAGCGGTATCAGCTCACTCAAAGGCGGTAATAC |
| | | GGTTATCCACAGAATCAGGGGATAACGCAGGAAAGAACATGTGAGCAAAAGGCCAGCAAAAGGCC |
| | | AGGAACCGTAAAAAGGCCGCGTTGCTGGCGTTTTTCCATAGGCTCCGCCCCCCTGACGAGCATCACA |
| | | AAAATCGACGCTCAAGTCAGAGGTGGCGAAACCCGACAGGACTATAAAGATACCAGGCGTTTCCCC |
| | | CTGGAAGCTCCCTCGTGCGCTCTCCTGTTCCGACCCTGCCGCTTACCGGATACCTGTCCGCCTTTCTCC |
| | | CTTCGGGAAGCGTGGCGCTTTCTCATAGCTCACGCTGTAGGTATCTCAGTTCGGTGTAGGTCGTTCGC |
| | | TCCAAGCTGGGCTGTGTGCACGAACCCCCCGTTCAGCCCGACCGCTGCGCCTTATCCGGTAACTATC |
| | | GTCTTGAGTCCAACCCGGTAAGACACGACTTATCGCCACTGGCAGCAGCCACTGGTAACAGGATTAG |
| | | CAGAGCGAGGTATGTAGGCGGTGCTACAGAGTTCTTGAAGTGGTGGCCTAACTACGGCTACACTAGA |
| | | AGAACAGTATTTGGTATCTGCGCTCTGCTGAAGCCAGTTACCTTCGGAAAAAGAGTTGGTAGCTCTT |
| | | GATCCGGCAAACAAACCACCGCTGGTAGCGGTGGTTTTTTTGTTTGCAAGCAGCAGATTACGCGCAG |
| | | AAAAAAAGGATCTCAAGAAGATCCTTTGATCTTTTCTACGGGGTCTGACGCTCAGTGGAACGAAAAC |
| | | TCACGTTAAGGGATTTTGGTCATGAGATTATCAAAAAGGATCTTCACCTAGATCCTTTTAAATTAAAA |
| | | ATGAAGTTTTAAATCAAGCCCAATCTGAATAATGTTACAACCAATTAACCAATTCTGATTAGAAAAA |
| | | CTCATCGAGCATCAAATGAAACTGCAATTTATTCATATCAGGATTATCAATACCATATTTTTGAAAAA |
| | | GCCGTTTCTGTAATGAAGGAGAAAACTCACCGAGGCAGTTCCATAGGATGGCAAGATCCTGGTATCG |
| | | GTCTGCGATTCCGACTCGTCCAACATCAATACAACCTATTAATTTCCCCTCGTCAAAAATAAGGTTAT |
| | | CAAGTGAGAAATCACCATGAGTGACGACTGAATCCGGTGAGAATGGCAAAAGTTTATGCATTTCTTT |
| | | CCAGACTTGTTCAACAGGCCAGCCATTACGCTCGTCATCAAAATCACTCGCATCAACCAAACCGTTA |
| | | TTCATTCGTGATTGCGCCTGAGCGAGACGAAATACGCGATCGCTGTTAAAAGGACAATTACAAACAG |
| | | GAATCGAATGCAACCGGCGCAGGAACACTGCCAGCGCATCAACAATATTTTCACCTGAATCAGGATA |
| | | TTCTTCTAATACCTGGAATGCTGTTTTTCCGGGGATCGCAGTGGTGAGTAACCATGCATCATCAGGAG |
| | | TACGGATAAAATGCTTGATGGTCGGAAGAGGCATAAATTCCGTCAGCCAGTTTAGTCTGACCATCTC |
| | | ATCTGTAACATCATTGGCAACGCTACCTTTGCCATGTTTCAGAAACAACTCTGGCGCATCGGGCTTCC |
| | | CATACAAGCGATAGATTGTCGCACCTGATTGCCCGACATTATCGCGAGCCCATTTATACCCATATAA |
| | | ATCAGCATCCATGTTGGAATTTAATCGCGGCCTCGACGTTTCCCGTTGAATATGGCTCATAACACCCC |
| | | TTGTATTACTGTTTATGTAAGCAGACAGTTTTATTGTTCATGATGATATATTTTTATCTTGTGCAATGT |
| | | AACATCAGAGATTTTGAGACACGGGCCAGAGCTGCATCGCGCGTTTCGGTGATGACGGTGAAAACCT |
| | | CTGACACATGCAGCTCCCGGAGACGGTCACAGCTTGTCTGTAAGCGGATGCCGGGAGCAGACAAGC |
| | | CCGTCAGGGCGCGTCAGCGGGTGTTGGCGGGTGTCGGGGCTGGCTTAACTATGCGGCATCAGAGCAG |
| | | ATTGTACTGAGAGTGCACGATATCGGGTCC |
| 37 | pAAV-hU6(318 bp)- | GTGCACGATATCGGGTCCCCAATTGCTGCGCGCTCGCTCGCTCACTGAGGCCGCCCGGGCAAAGCCC |
| | SGN004282-EFS- | GGGCGTCGGGCGACCTTTGGTCGCCCGGCCTCAGTGAGCGAGCGAGCGCGCAGAGAGGGAGTGGCC |
| | APG05586mco- | AACTCCATCACTAGGGGTTCCTCGTCGACAATGCACGCGTTCGAAGTACTCATGTACAAAAAAGCAG |
| | SV40pA(179 bp) | GCTTTAAAGGAACCAATTCAGTCGACTGGATCCGGTACCAAGGTCGGGCAGGAAGAGGGCCTATTTC |
| | | CCATGATTCCTTCATATTTGCATATACGATACAAGGCTGTTAGAGAGATAATTAGAATTAATTTGACT |
| | | GTAAACACAAAGATATTAGTACAAAATACGTGACGTAGAAAGTAATAATTTCTTGGGTAGTTTGCAG |
| | | TTTTAAAATTATGTTTTAAAATGGACTATCATATGCTTACCGTAACTTGAAAGTATTTCGATTTCTTGG |
| | | CTTTATATATCTTGTGGAAAGGACGAAACACCGACCGGCCTCCAGGATGAAGTGCACAGTTATTGTA |
| | | CTCAAAGGAATCTACAATAATAAGGCATCTTGCCGAATTTACCGCCCTACATATGTAGGGCGGTTTTT |
| | | TTTTGTCTAGACAAGTTGGAGCTCGAATTCGCTAGCTAGGTCTTGAAAGGAGTGGGAATTGGCTCCG |
| | | GTGCCCGTCAGTGGGCAGAGCGCACATCGCCCACAGTCCCCGAGAAGTTGGGGGGAGGGGTCGGCA |
| | | ATTGATCCGGTGCCTAGAGAAGGTGGCGCGGGGTAAACTGGGAAAGTGATGTCGTGTACTGGCTCCG |
| | | CCTTTTTCCCGAGGGTGGGGGAGAACCGTATATAAGTGCAGTAGTCGCCGTGAACGTTCTTTTTCGCA |
| | | ACGGGTTTGCCGCCAGAACACAGGACCGGTGCCACCGCCACCATGGCCCCTAAGAAGAAAAGAAAG |
| | | GTGATGTACTCTATCGGCCTCGATCTGGGCATCTCTAGCGTCGGCTGGTCGGTGATCGACGAAGAGA |
| | | CAGGCAAAATCGTTGACCTGGGCGTGCGGCTGTTTTCTGCTAAGAACAGCGAGAAGAATCTGGAGA |
| | | GAAGAACCAGCAGAGGCGCAAGAAGACTGATCAGAAGAAAGACGAACCGACTAAAAGACGCCAAG |
| | | AAATTACTGGAGGCCATCGGCTTCTACGAAGATAAGGCCCTTAAAAATGTCTGCCCTTACCAACTGA |
| | | GAGTGAAGGGCCTGACCGAGGGCCTGACAAAGGGCGAACTGTACAAGGTCGTGCTGCACATCGTGA |
| | | AGAAACGGGGCATCAGCTACCTGGACGAGGACGACGCCGAAGCTGCCAAAGAATCTCAGGACTACA |
| | | AGGAGCAGGTACGCAAAAATGCCCAACTGCTGACAAAGTACACCCCTGGACAGATCCAACTGCAGA |
| | | GACTGAAGGAGAACAACAGAGTGAAGACAGGAATCAACGGCCAGGGCCACTACCAACTGAACGTGT |
| | | TCAAGGTGAGCGCTTACGCTGACGAGCTGGCCACCATCCTGAAGACCCAGCAAGCCCTGTATCCTAA |
| | | CGAACTGACCGACGACTGGATCGCCCTGTTCGTGCAACCTGGAATTGCCGAGAATGCCGGCCTTATC |
| | | TACAGAAAGAGGCCTTACTACCACGGCCCTGGCAACGAGGCCAACAACAGCCCTTACGGAAGATGG |
| | | TCCGACTTCCAGAAAACAGGCCAGCCAGCCGCTAACATCTTCGACAAGCTAATCGGAAAGGATTTCC |
| | | AGGGCGAGCTGCGGGCCAGCGGCCTGAGCCTGTCTGCCCAGCAATACAACCTGCTGAACGACCTGA |
| | | CCAATCTGAAAATTGACGGAGAGGTGTCCCTGTCCCCCGAGCAGAAGGAATTCATCCTGACCGAGCT |
| | | GATGACCAAGGAATTCGCCAGATTCGGCGTCAACGACATTGCCAAGCTGCTGGGCGTGAAAAAAGA |
| | | ACAGTTGTCTGGCTGGCGGCTGGACAAGAAGGGCAAGCCCGAAATCCACACCCTGAAGGGCTACAG |
| | | AAACTGGAGAAAGATCTTTGCCGAGGCCGGAATCGACCTGGCCACACTGCCAACAGAGACCATTGA |
| | | CTGCCTGGCCAAGGTTCTGACCCTGAACACCGAGCGGGAAGGAGTGGAAAACACCCTGGCCTTTGA |
| | | GCTGCCTGAACTGGCTGAGCCTGTGAAGAGCCTGGTGTTGGACCATTACAAGGAACTGAGCCAAAGC |
| | | ATCTCAACACAGGCTTGGCACAGATTCAGCCTGAAGACACTGCACCTGCTGATCCCCGAGCTGATCA |
| | | AGAGCACCAGCGAGCAGAACACCCTGCTGGAACAGTTCCAACTGAAGGCCGGCGTGCGCAAGCGGT |
| | | ACTCCGACTACAAGAAGCTGCCTACCAAGGAGGTGCTGGCCGAGATCTACAATCCTACTGTGAACAA |
| | | AACCGTCAGCCAGGCCTTCAAGGTGATGGACGCTCTGCTGGAGAAGTACGGCAAGGACCAGATCCA |
| | | CTACATCACCGTGGAAATGCCTAGAGATGATAACGAGGAGGAAGAGCGTAAAAGAATTAAGGAACT |
| | | GCAAACGAAGAATTCTCAGAGAAAGAACGATAGCCAGCAATACTTCCTGCAGAAATCCGGCTGGAG |
| | | CCAGGAGAAGTTCCAGGCCACAATCCACAAGAATAGAAGATTCCTGGCCAAGCTGCTGTACTATTTT |
| | | GAGCAGGACGGCGTGTGTGCCTACACCGGGAACCCCATCAGCCCTGAGCTTCTGGTGTCCGACAGCA |
| | | CCGAGATCGACCACATCATCCCTATCAGCATCAGCCTGGATGACAGCATCAACAACAAAGTGCTGGT |
| | | GCTGTCTCACGCCAACCAGGTGAAGGGCCAACAGACCCCTTACGATGCCAGAATGGCCGGCGCCTTC |
| | | AACAAGATCAATGGAAAGTTCAGCAACTGGGACGAGTATCAAAAGTGGGTGGAAAGCAGACCTTTC |
| | | AGCCGGAAAAAGGTGAACAACCTCCTGGAGACCAGAAACATTTTCGACAGCGAGCAGGTGCAAAAG |
| | | TTTCTGAGCAGAAACCTGAATGATACAAGATACGCCAGCAGACTGGTCCTGAATACCCTGCAGAGCT |
| | | TTTTCGAGAACCAGGATACCATTGTGCGGGTGGTCAACGGCAGCTTCACCCATACCCTGAGAAAGAA |
| | | GTGGGGCGCCGATCTGGACAAAACCCGGGAGACACACCACCACCACGCCGTGGACGCCACACTGTG |
| | | CGCGGTGACACCCTTCGTGAAGGTCTCTAGATACCACTACGCCGTGAACGAGGAGACCGGCGAGAA |
| | | ATTCATGAGAGAAATCGATGTGGAGACCGGCGAAATCCTGGACGAAATTCCATACAGAGAGTACAA |
| | | GAAGGCCAAGCACTACGAGAGAAAAACCTACCAGGTGAAGTGGTCCAACTTCCGCGAGCAGCTTAA |
| | | GCCCATCACAATCCATCCTAAGATCAAGTTCAGCCACCAGGTTGACAGAAAAGCCAACAGAAAGCT |
| | | GAGCGACGCTACCATCTACTCCGTTAGAGAGAAGACAGAGGTGAAAACACTGAAGAGCGGCAAGGA |
| | | GAAGATCACGACCGACGAATACACCATCGGCAAGATCAAGGACATCTATACCGTAGACGGCTGGGA |
| | | AGCCTTTAAAAAGAAGCAGGACAAACTGCTCATGAAGGAATTCGATGAAAAGACCTACGAGCTGCT |
| | | GGTGACCATCGCCGCCACCACCCCTGATTTTCAGGAGGTGGAAGAAAAGAACGGCAAAGTGAAGAG |
| | | AGTGAAGCGGAGCCCTTTCGCCGTGTACTGTGAGGAAAACGGCATACCCGCCATCCGGAAATACGCC |
| | | AAGAAGAACAACGGCCCCGTGATCCGGAGCCTGAAGTACTACGACGGCAAGCTGAATAAGCACATC |
| | | AACATCACCAAGGATGAGAAGGGCAGGCCCGTGGAGCAGACAAAGAACGGCCGGAAAGTGACTCT |
| | | GCAGAGCCTGAAACCCTACCGGTATGATATCTACCAGGACCTCGAAACCAAGGCCTATTATACAGTG |
| | | CAACTGTACTACAGCGACCTGCGCTTTGTGGAGGGCGAGTACGGAATCACAGAAAAAGAGTACATG |
| | | AAGAAGGTGGCCGAACAGACCAAAGGACAGGTGGTGCGGTTCTGCTTCAGCCTGCAGAAGAACGAC |
| | | GGCCTCGAAATCGAGTGGAAGGATTCTCAACGGTACGACGTGAGATTCTACAATTTCCAATCTGCTA |
| | | ACAGCATCAACTTCAAGGGCTTAGAACAGGAGATGATCCCTGCTGAAAACCAGTTCAAGCAGAAGC |
| | | CTTACAACAATGGCGCCATCAACCTGAACATCGCCAAGTATGGCAAGGAGGGCAAGAAACTGAGAA |
| | | AGTTCAACACCGATATCCTGGGTAAAAAGCACCACCTGTCTTATGAGAAAGAGCCGAAGAACATCAT |
| | | CAAGAGCGGCGGAAGCAAAAGACCTGCCGCTACAAAGAAGGCCGGCCAGGCCAAGAAAAAGAAGT |
| | | AAGCTAGCTTGACTGACTGAGATACAGCGTACCTTCAGCTCACAGACATGATAAGATACATTGATGA |
| | | GTTTGGACAAACCACAACTAGAATGCAGTGAAAAAAATGCTTTATTTGTGAAATTTGTGATGCTATT |
| | | GCTTTATTTGTAACCATTATAAGCTGCAATAAACAAGTTAACAACAACAATTGCATTCATTTTATGTT |
| | | TCAGGTTCAGGGGGAGGTGTGGGAGGTTTTTTAAAGCAAGTAAAAGCTTGGTAGGAACCCCTAGTGA |
| | | TGGAGTTGGCCACTCCCTCTCTGCGCGCTCGCTCGCTCACTGAGGCCGGGCGACCAAAGGTCGCCCG |
| | | ACGCCCGGGCTTTGCCCGGGCGGCCTCAGTGAGCGAGCGAGCGCGCAGAGGCATTTAATTAAGCAA |
| | | GCTGTCATAGCTGTTTCCTGTGTGAAATTGTTATCCGCTCACAATTCCACACAACATACGAGCCGGAA |
| | | GCATAAAGTGTAAAGCCTGGGGTGCCTAATGAGTGAGCTAACTCACATTAATTGCGTTGCGCTCACT |
| | | GCCCGCTTTCCAGTCGGGAAACCTGTCGTGCCAGCTGCATTAATGAATCGGCCAACGCGCGGGGAGA |
| | | GGCGGTTTGCGTATTGGGCGCTCTTCCGCTTCCTCGCTCACTGACTCGCTGCGCTCGGTCGTTCGGCT |
| | | GCGGCGAGCGGTATCAGCTCACTCAAAGGCGGTAATACGGTTATCCACAGAATCAGGGGATAACGC |
| | | AGGAAAGAACATGTGAGCAAAAGGCCAGCAAAAGGCCAGGAACCGTAAAAAGGCCGCGTTGCTGG |
| | | CGTTTTTCCATAGGCTCCGCCCCCCTGACGAGCATCACAAAAATCGACGCTCAAGTCAGAGGTGGCG |
| | | AAACCCGACAGGACTATAAAGATACCAGGCGTTTCCCCCTGGAAGCTCCCTCGTGCGCTCTCCTGTT |
| | | CCGACCCTGCCGCTTACCGGATACCTGTCCGCCTTTCTCCCTTCGGGAAGCGTGGCGCTTTCTCATAG |
| | | CTCACGCTGTAGGTATCTCAGTTCGGTGTAGGTCGTTCGCTCCAAGCTGGGCTGTGTGCACGAACCCC |
| | | CCGTTCAGCCCGACCGCTGCGCCTTATCCGGTAACTATCGTCTTGAGTCCAACCCGGTAAGACACGA |
| | | CTTATCGCCACTGGCAGCAGCCACTGGTAACAGGATTAGCAGAGCGAGGTATGTAGGCGGTGCTACA |
| | | GAGTTCTTGAAGTGGTGGCCTAACTACGGCTACACTAGAAGAACAGTATTTGGTATCTGCGCTCTGC |
| | | TGAAGCCAGTTACCTTCGGAAAAAGAGTTGGTAGCTCTTGATCCGGCAAACAAACCACCGCTGGTAG |
| | | CGGTGGTTTTTTTGTTTGCAAGCAGCAGATTACGCGCAGAAAAAAAGGATCTCAAGAAGATCCTTTG |
| | | ATCTTTTCTACGGGGTCTGACGCTCAGTGGAACGAAAACTCACGTTAAGGGATTTTGGTCATGAGAT |
| | | TATCAAAAAGGATCTTCACCTAGATCCTTTTAAATTAAAAATGAAGTTTTAAATCAAGCCCAATCTG |
| | | AATAATGTTACAACCAATTAACCAATTCTGATTAGAAAAACTCATCGAGCATCAAATGAAACTGCAA |
| | | TTTATTCATATCAGGATTATCAATACCATATTTTTGAAAAAGCCGTTTCTGTAATGAAGGAGAAAACT |
| | | CACCGAGGCAGTTCCATAGGATGGCAAGATCCTGGTATCGGTCTGCGATTCCGACTCGTCCAACATC |
| | | AATACAACCTATTAATTTCCCCTCGTCAAAAATAAGGTTATCAAGTGAGAAATCACCATGAGTGACG |
| | | ACTGAATCCGGTGAGAATGGCAAAAGTTTATGCATTTCTTTCCAGACTTGTTCAACAGGCCAGCCATT |
| | | ACGCTCGTCATCAAAATCACTCGCATCAACCAAACCGTTATTCATTCGTGATTGCGCCTGAGCGAGA |
| | | CGAAATACGCGATCGCTGTTAAAAGGACAATTACAAACAGGAATCGAATGCAACCGGCGCAGGAAC |
| | | ACTGCCAGCGCATCAACAATATTTTCACCTGAATCAGGATATTCTTCTAATACCTGGAATGCTGTTTT |
| | | TCCGGGGATCGCAGTGGTGAGTAACCATGCATCATCAGGAGTACGGATAAAATGCTTGATGGTCGGA |
| | | AGAGGCATAAATTCCGTCAGCCAGTTTAGTCTGACCATCTCATCTGTAACATCATTGGCAACGCTACC |
| | | TTTGCCATGTTTCAGAAACAACTCTGGCGCATCGGGCTTCCCATACAAGCGATAGATTGTCGCACCTG |
| | | ATTGCCCGACATTATCGCGAGCCCATTTATACCCATATAAATCAGCATCCATGTTGGAATTTAATCGC |
| | | GGCCTCGACGTTTCCCGTTGAATATGGCTCATAACACCCCTTGTATTACTGTTTATGTAAGCAGACAG |
| | | TTTTATTGTTCATGATGATATATTTTTATCTTGTGCAATGTAACATCAGAGATTTTGAGACACGGGCC |
| | | AGAGCTGCATCGCGCGTTTCGGTGATGACGGTGAAAACCTCTGACACATGCAGCTCCCGGAGACGGT |
| | | CACAGCTTGTCTGTAAGCGGATGCCGGGAGCAGACAAGCCCGTCAGGGCGCGTCAGCGGGTGTTGG |
| | | CGGGTGTCGGGGCTGGCTTAACTATGCGGCATCAGAGCAGATTGTACTGAGAGTGCACGATATCGGG |
| | | TCC |
| 38 | pAAV-hU6(249 bp)- | GTGCACGATATCGGGTCCCCAATTGCTGCGCGCTCGCTCGCTCACTGAGGCCGCCCGGGCAAAGCCC |
| | SGN004282- | GGGCGTCGGGCGACCTTTGGTCGCCCGGCCTCAGTGAGCGAGCGAGCGCGCAGAGAGGGAGTGGCC |
| | CMVeb- | AACTCCATCACTAGGGGTTCCTCGTCGACAATGCACGCGTTCGAAGTACTCAGAGGGCCTATTTCCC |
| | APG05586mco- | ATGATTCCTTCATATTTGCATATACGATACAAGGCTGTTAGAGAGATAATTGGAATTAATTTGACTGT |
| | bGHpA(225 bp) | AAACACAAAGATATTAGTACAAAATACGTGACGTAGAAAGTAATAATTTCTTGGGTAGTTTGCAGTT |
| | | TTAAAATTATGTTTTAAAATGGACTATCATATGCTTACCGTAACTTGAAAGTATTTCGATTTCTTGGC |
| | | TTTATATATCTTGTGGAAAGGACGAAACACCGACCGGCCTCCAGGATGAAGTGCACAGTTATTGTAC |
| | | TCAAAGGAATCTACAATAATAAGGCATCTTGCCGAATTTACCGCCCTACATATGTAGGGCGGTTTTTT |
| | | TTTGTCTAGACAAGTTGGAGCTCGTGATGCGGTTTTGGCAGTACATCAATGGGCGTGGATAGCGGTT |
| | | TGACTCACGGGGATTTCCAAGTCTCCACCCCATTGACGTCAATGGGAGTTTGTTTTGGCACCAAAATC |
| | | AACGGGACTTTCCAAAATGTCGTAACAACTCCGCCCCATTGACGCAAATGGGCGGTAGGCGTGTACG |
| | | GTGGGAGGTCTATATAAGCAGAGCTCGTTTAGTGAACCGTCAGATCGCCTCTTTGTCGAGCCACCAT |
| | | GGCCCCTAAGAAGAAAAGAAAGGTGATGTACTCTATCGGCCTCGATCTGGGCATCTCTAGCGTCGGC |
| | | TGGTCGGTGATCGACGAAGAGACAGGCAAAATCGTTGACCTGGGCGTGCGGCTGTTTTCTGCTAAGA |
| | | ACAGCGAGAAGAATCTGGAGAGAAGAACCAGCAGAGGCGCAAGAAGACTGATCAGAAGAAAGACG |
| | | AACCGACTAAAAGACGCCAAGAAATTACTGGAGGCCATCGGCTTCTACGAAGATAAGGCCCTTAAA |
| | | AATGTCTGCCCTTACCAACTGAGAGTGAAGGGCCTGACCGAGGGCCTGACAAAGGGCGAACTGTAC |
| | | AAGGTCGTGCTGCACATCGTGAAGAAACGGGGCATCAGCTACCTGGACGAGGACGACGCCGAAGCT |
| | | GCCAAAGAATCTCAGGACTACAAGGAGCAGGTACGCAAAAATGCCCAACTGCTGACAAAGTACACC |
| | | CCTGGACAGATCCAACTGCAGAGACTGAAGGAGAACAACAGAGTGAAGACAGGAATCAACGGCCA |
| | | GGGCCACTACCAACTGAACGTGTTCAAGGTGAGCGCTTACGCTGACGAGCTGGCCACCATCCTGAAG |
| | | ACCCAGCAAGCCCTGTATCCTAACGAACTGACCGACGACTGGATCGCCCTGTTCGTGCAACCTGGAA |
| | | TTGCCGAGAATGCCGGCCTTATCTACAGAAAGAGGCCTTACTACCACGGCCCTGGCAACGAGGCCAA |
| | | CAACAGCCCTTACGGAAGATGGTCCGACTTCCAGAAAACAGGCCAGCCAGCCGCTAACATCTTCGAC |
| | | AAGCTAATCGGAAAGGATTTCCAGGGCGAGCTGCGGGCCAGCGGCCTGAGCCTGTCTGCCCAGCAA |
| | | TACAACCTGCTGAACGACCTGACCAATCTGAAAATTGACGGAGAGGTGTCCCTGTCCCCCGAGCAGA |
| | | AGGAATTCATCCTGACCGAGCTGATGACCAAGGAATTCGCCAGATTCGGCGTCAACGACATTGCCAA |
| | | GCTGCTGGGCGTGAAAAAAGAACAGTTGTCTGGCTGGCGGCTGGACAAGAAGGGCAAGCCCGAAAT |
| | | CCACACCCTGAAGGGCTACAGAAACTGGAGAAAGATCTTTGCCGAGGCCGGAATCGACCTGGCCAC |
| | | ACTGCCAACAGAGACCATTGACTGCCTGGCCAAGGTTCTGACCCTGAACACCGAGCGGGAAGGAGT |
| | | GGAAAACACCCTGGCCTTTGAGCTGCCTGAACTGGCTGAGCCTGTGAAGAGCCTGGTGTTGGACCAT |
| | | TACAAGGAACTGAGCCAAAGCATCTCAACACAGGCTTGGCACAGATTCAGCCTGAAGACACTGCAC |
| | | CTGCTGATCCCCGAGCTGATCAAGAGCACCAGCGAGCAGAACACCCTGCTGGAACAGTTCCAACTGA |
| | | AGGCCGGCGTGCGCAAGCGGTACTCCGACTACAAGAAGCTGCCTACCAAGGAGGTGCTGGCCGAGA |
| | | TCTACAATCCTACTGTGAACAAAACCGTCAGCCAGGCCTTCAAGGTGATGGACGCTCTGCTGGAGAA |
| | | GTACGGCAAGGACCAGATCCACTACATCACCGTGGAAATGCCTAGAGATGATAACGAGGAGGAAGA |
| | | GCGTAAAAGAATTAAGGAACTGCAAACGAAGAATTCTCAGAGAAAGAACGATAGCCAGCAATACTT |
| | | CCTGCAGAAATCCGGCTGGAGCCAGGAGAAGTTCCAGGCCACAATCCACAAGAATAGAAGATTCCT |
| | | GGCCAAGCTGCTGTACTATTTTGAGCAGGACGGCGTGTGTGCCTACACCGGGAACCCCATCAGCCCT |
| | | GAGCTTCTGGTGTCCGACAGCACCGAGATCGACCACATCATCCCTATCAGCATCAGCCTGGATGACA |
| | | GCATCAACAACAAAGTGCTGGTGCTGTCTCACGCCAACCAGGTGAAGGGCCAACAGACCCCTTACG |
| | | ATGCCAGAATGGCCGGCGCCTTCAACAAGATCAATGGAAAGTTCAGCAACTGGGACGAGTATCAAA |
| | | AGTGGGTGGAAAGCAGACCTTTCAGCCGGAAAAAGGTGAACAACCTCCTGGAGACCAGAAACATTT |
| | | TCGACAGCGAGCAGGTGCAAAAGTTTCTGAGCAGAAACCTGAATGATACAAGATACGCCAGCAGAC |
| | | TGGTCCTGAATACCCTGCAGAGCTTTTTCGAGAACCAGGATACCATTGTGCGGGTGGTCAACGGCAG |
| | | CTTCACCCATACCCTGAGAAAGAAGTGGGGCGCCGATCTGGACAAAACCCGGGAGACACACCACCA |
| | | CCACGCCGTGGACGCCACACTGTGCGCGGTGACACCCTTCGTGAAGGTCTCTAGATACCACTACGCC |
| | | GTGAACGAGGAGACCGGCGAGAAATTCATGAGAGAAATCGATGTGGAGACCGGCGAAATCCTGGAC |
| | | GAAATTCCATACAGAGAGTACAAGAAGGCCAAGCACTACGAGAGAAAAACCTACCAGGTGAAGTGG |
| | | TCCAACTTCCGCGAGCAGCTTAAGCCCATCACAATCCATCCTAAGATCAAGTTCAGCCACCAGGTTG |
| | | ACAGAAAAGCCAACAGAAAGCTGAGCGACGCTACCATCTACTCCGTTAGAGAGAAGACAGAGGTGA |
| | | AAACACTGAAGAGCGGCAAGGAGAAGATCACGACCGACGAATACACCATCGGCAAGATCAAGGAC |
| | | ATCTATACCGTAGACGGCTGGGAAGCCTTTAAAAAGAAGCAGGACAAACTGCTCATGAAGGAATTC |
| | | GATGAAAAGACCTACGAGCTGCTGGTGACCATCGCCGCCACCACCCCTGATTTTCAGGAGGTGGAAG |
| | | AAAAGAACGGCAAAGTGAAGAGAGTGAAGCGGAGCCCTTTCGCCGTGTACTGTGAGGAAAACGGCA |
| | | TACCCGCCATCCGGAAATACGCCAAGAAGAACAACGGCCCCGTGATCCGGAGCCTGAAGTACTACG |
| | | ACGGCAAGCTGAATAAGCACATCAACATCACCAAGGATGAGAAGGGCAGGCCCGTGGAGCAGACA |
| | | AAGAACGGCCGGAAAGTGACTCTGCAGAGCCTGAAACCCTACCGGTATGATATCTACCAGGACCTC |
| | | GAAACCAAGGCCTATTATACAGTGCAACTGTACTACAGCGACCTGCGCTTTGTGGAGGGCGAGTACG |
| | | GAATCACAGAAAAAGAGTACATGAAGAAGGTGGCCGAACAGACCAAAGGACAGGTGGTGCGGTTCT |
| | | GCTTCAGCCTGCAGAAGAACGACGGCCTCGAAATCGAGTGGAAGGATTCTCAACGGTACGACGTGA |
| | | GATTCTACAATTTCCAATCTGCTAACAGCATCAACTTCAAGGGCTTAGAACAGGAGATGATCCCTGC |
| | | TGAAAACCAGTTCAAGCAGAAGCCTTACAACAATGGCGCCATCAACCTGAACATCGCCAAGTATGG |
| | | CAAGGAGGGCAAGAAACTGAGAAAGTTCAACACCGATATCCTGGGTAAAAAGCACCACCTGTCTTA |
| | | TGAGAAAGAGCCGAAGAACATCATCAAGAGCGGCGGAAGCAAAAGACCTGCCGCTACAAAGAAGG |
| | | CCGGCCAGGCCAAGAAAAAGAAGTAAGCTAGCTTGACTGACTGAGATACAGCGTACCTTCAGCTCA |
| | | CAGACATGACTGTGCCTTCTAGTTGCCAGCCATCTGTTGTTTGCCCCTCCCCCGTGCCTTCCTTGACCC |
| | | TGGAAGGTGCCACTCCCACTGTCCTTTCCTAATAAAATGAGGAAATTGCATCGCATTGTCTGAGTAG |
| | | GTGTCATTCTATTCTGGGGGGTGGGGTGGGGCAGGACAGCAAGGGGGAGGATTGGGAAGACAATAG |
| | | CAGGCATGCTGGGGATGCGGTGGGCTCTATGGTTTTAAAGCAAGTAAAAGCTTGGTAGGAACCCCTA |
| | | GTGATGGAGTTGGCCACTCCCTCTCTGCGCGCTCGCTCGCTCACTGAGGCCGGGCGACCAAAGGTCG |
| | | CCCGACGCCCGGGCTTTGCCCGGGCGGCCTCAGTGAGCGAGCGAGCGCGCAGAGGCATTTAATTAA |
| | | GCAAGCTGTCATAGCTGTTTCCTGTGTGAAATTGTTATCCGCTCACAATTCCACACAACATACGAGCC |
| | | GGAAGCATAAAGTGTAAAGCCTGGGGTGCCTAATGAGTGAGCTAACTCACATTAATTGCGTTGCGCT |
| | | CACTGCCCGCTTTCCAGTCGGGAAACCTGTCGTGCCAGCTGCATTAATGAATCGGCCAACGCGCGGG |
| | | GAGAGGCGGTTTGCGTATTGGGCGCTCTTCCGCTTCCTCGCTCACTGACTCGCTGCGCTCGGTCGTTC |
| | | GGCTGCGGCGAGCGGTATCAGCTCACTCAAAGGCGGTAATACGGTTATCCACAGAATCAGGGGATA |
| | | ACGCAGGAAAGAACATGTGAGCAAAAGGCCAGCAAAAGGCCAGGAACCGTAAAAAGGCCGCGTTG |
| | | CTGGCGTTTTTCCATAGGCTCCGCCCCCCTGACGAGCATCACAAAAATCGACGCTCAAGTCAGAGGT |
| | | GGCGAAACCCGACAGGACTATAAAGATACCAGGCGTTTCCCCCTGGAAGCTCCCTCGTGCGCTCTCC |
| | | TGTTCCGACCCTGCCGCTTACCGGATACCTGTCCGCCTTTCTCCCTTCGGGAAGCGTGGCGCTTTCTC |
| | | ATAGCTCACGCTGTAGGTATCTCAGTTCGGTGTAGGTCGTTCGCTCCAAGCTGGGCTGTGTGCACGA |
| | | ACCCCCCGTTCAGCCCGACCGCTGCGCCTTATCCGGTAACTATCGTCTTGAGTCCAACCCGGTAAGAC |
| | | ACGACTTATCGCCACTGGCAGCAGCCACTGGTAACAGGATTAGCAGAGCGAGGTATGTAGGCGGTG |
| | | CTACAGAGTTCTTGAAGTGGTGGCCTAACTACGGCTACACTAGAAGAACAGTATTTGGTATCTGCGC |
| | | TCTGCTGAAGCCAGTTACCTTCGGAAAAAGAGTTGGTAGCTCTTGATCCGGCAAACAAACCACCGCT |
| | | GGTAGCGGTGGTTTTTTTGTTTGCAAGCAGCAGATTACGCGCAGAAAAAAAGGATCTCAAGAAGATC |
| | | CTTTGATCTTTTCTACGGGGTCTGACGCTCAGTGGAACGAAAACTCACGTTAAGGGATTTTGGTCATG |
| | | AGATTATCAAAAAGGATCTTCACCTAGATCCTTTTAAATTAAAAATGAAGTTTTAAATCAAGCCCAA |
| | | TCTGAATAATGTTACAACCAATTAACCAATTCTGATTAGAAAAACTCATCGAGCATCAAATGAAACT |
| | | GCAATTTATTCATATCAGGATTATCAATACCATATTTTTGAAAAAGCCGTTTCTGTAATGAAGGAGAA |
| | | AACTCACCGAGGCAGTTCCATAGGATGGCAAGATCCTGGTATCGGTCTGCGATTCCGACTCGTCCAA |
| | | CATCAATACAACCTATTAATTTCCCCTCGTCAAAAATAAGGTTATCAAGTGAGAAATCACCATGAGT |
| | | GACGACTGAATCCGGTGAGAATGGCAAAAGTTTATGCATTTCTTTCCAGACTTGTTCAACAGGCCAG |
| | | CCATTACGCTCGTCATCAAAATCACTCGCATCAACCAAACCGTTATTCATTCGTGATTGCGCCTGAGC |
| | | GAGACGAAATACGCGATCGCTGTTAAAAGGACAATTACAAACAGGAATCGAATGCAACCGGCGCAG |
| | | GAACACTGCCAGCGCATCAACAATATTTTCACCTGAATCAGGATATTCTTCTAATACCTGGAATGCTG |
| | | TTTTTCCGGGGATCGCAGTGGTGAGTAACCATGCATCATCAGGAGTACGGATAAAATGCTTGATGGT |
| | | CGGAAGAGGCATAAATTCCGTCAGCCAGTTTAGTCTGACCATCTCATCTGTAACATCATTGGCAACG |
| | | CTACCTTTGCCATGTTTCAGAAACAACTCTGGCGCATCGGGCTTCCCATACAAGCGATAGATTGTCGC |
| | | ACCTGATTGCCCGACATTATCGCGAGCCCATTTATACCCATATAAATCAGCATCCATGTTGGAATTTA |
| | | ATCGCGGCCTCGACGTTTCCCGTTGAATATGGCTCATAACACCCCTTGTATTACTGTTTATGTAAGCA |
| | | GACAGTTTTATTGTTCATGATGATATATTTTTATCTTGTGCAATGTAACATCAGAGATTTTGAGACAC |
| | | GGGCCAGAGCTGCATCGCGCGTTTCGGTGATGACGGTGAAAACCTCTGACACATGCAGCTCCCGGAG |
| | | ACGGTCACAGCTTGTCTGTAAGCGGATGCCGGGAGCAGACAAGCCCGTCAGGGCGCGTCAGCGGGT |
| | | GTTGGCGGGTGTCGGGGCTGGCTTAACTATGCGGCATCAGAGCAGATTGTACTGAGAGTGCACGATA |
| | | TCGGGTCC |
| 39 | pAAV-hU6(249 bp)- | GTGCACGATATCGGGTCCCCAATTGCTGCGCGCTCGCTCGCTCACTGAGGCCGCCCGGGCAAAGCCC |
| | SGN004282-EFS- | GGGCGTCGGGCGACCTTTGGTCGCCCGGCCTCAGTGAGCGAGCGAGCGCGCAGAGAGGGAGTGGCC |
| | APG05586mco- | AACTCCATCACTAGGGGTTCCTCGTCGACAATGCACGCGTTCGAAGTACTCAGAGGGCCTATTTCCC |
| | bGHpA(225 bp) | ATGATTCCTTCATATTTGCATATACGATACAAGGCTGTTAGAGAGATAATTGGAATTAATTTGACTGT |
| | | AAACACAAAGATATTAGTACAAAATACGTGACGTAGAAAGTAATAATTTCTTGGGTAGTTTGCAGTT |
| | | TTAAAATTATGTTTTAAAATGGACTATCATATGCTTACCGTAACTTGAAAGTATTTCGATTTCTTGGC |
| | | TTTATATATCTTGTGGAAAGGACGAAACACCGACCGGCCTCCAGGATGAAGTGCACAGTTATTGTAC |
| | | TCAAAGGAATCTACAATAATAAGGCATCTTGCCGAATTTACCGCCCTACATATGTAGGGCGGTTTTTT |
| | | TTTGTCTAGACAAGTTGGAGCTCGAATTCGCTAGCTAGGTCTTGAAAGGAGTGGGAATTGGCTCCGG |
| | | TGCCCGTCAGTGGGCAGAGCGCACATCGCCCACAGTCCCCGAGAAGTTGGGGGGAGGGGTCGGCAA |
| | | TTGATCCGGTGCCTAGAGAAGGTGGCGCGGGGTAAACTGGGAAAGTGATGTCGTGTACTGGCTCCGC |
| | | CTTTTTCCCGAGGGTGGGGGAGAACCGTATATAAGTGCAGTAGTCGCCGTGAACGTTCTTTTTCGCA |
| | | ACGGGTTTGCCGCCAGAACACAGGACCGGTGCCACCGCCACCATGGCCCCTAAGAAGAAAAGAAAG |
| | | GTGATGTACTCTATCGGCCTCGATCTGGGCATCTCTAGCGTCGGCTGGTCGGTGATCGACGAAGAGA |
| | | CAGGCAAAATCGTTGACCTGGGCGTGCGGCTGTTTTCTGCTAAGAACAGCGAGAAGAATCTGGAGA |
| | | GAAGAACCAGCAGAGGCGCAAGAAGACTGATCAGAAGAAAGACGAACCGACTAAAAGACGCCAAG |
| | | AAATTACTGGAGGCCATCGGCTTCTACGAAGATAAGGCCCTTAAAAATGTCTGCCCTTACCAACTGA |
| | | GAGTGAAGGGCCTGACCGAGGGCCTGACAAAGGGCGAACTGTACAAGGTCGTGCTGCACATCGTGA |
| | | AGAAACGGGGCATCAGCTACCTGGACGAGGACGACGCCGAAGCTGCCAAAGAATCTCAGGACTACA |
| | | AGGAGCAGGTACGCAAAAATGCCCAACTGCTGACAAAGTACACCCCTGGACAGATCCAACTGCAGA |
| | | GACTGAAGGAGAACAACAGAGTGAAGACAGGAATCAACGGCCAGGGCCACTACCAACTGAACGTGT |
| | | TCAAGGTGAGCGCTTACGCTGACGAGCTGGCCACCATCCTGAAGACCCAGCAAGCCCTGTATCCTAA |
| | | CGAACTGACCGACGACTGGATCGCCCTGTTCGTGCAACCTGGAATTGCCGAGAATGCCGGCCTTATC |
| | | TACAGAAAGAGGCCTTACTACCACGGCCCTGGCAACGAGGCCAACAACAGCCCTTACGGAAGATGG |
| | | TCCGACTTCCAGAAAACAGGCCAGCCAGCCGCTAACATCTTCGACAAGCTAATCGGAAAGGATTTCC |
| | | AGGGCGAGCTGCGGGCCAGCGGCCTGAGCCTGTCTGCCCAGCAATACAACCTGCTGAACGACCTGA |
| | | CCAATCTGAAAATTGACGGAGAGGTGTCCCTGTCCCCCGAGCAGAAGGAATTCATCCTGACCGAGCT |
| | | GATGACCAAGGAATTCGCCAGATTCGGCGTCAACGACATTGCCAAGCTGCTGGGCGTGAAAAAAGA |
| | | ACAGTTGTCTGGCTGGCGGCTGGACAAGAAGGGCAAGCCCGAAATCCACACCCTGAAGGGCTACAG |
| | | AAACTGGAGAAAGATCTTTGCCGAGGCCGGAATCGACCTGGCCACACTGCCAACAGAGACCATTGA |
| | | CTGCCTGGCCAAGGTTCTGACCCTGAACACCGAGCGGGAAGGAGTGGAAAACACCCTGGCCTTTGA |
| | | GCTGCCTGAACTGGCTGAGCCTGTGAAGAGCCTGGTGTTGGACCATTACAAGGAACTGAGCCAAAGC |
| | | ATCTCAACACAGGCTTGGCACAGATTCAGCCTGAAGACACTGCACCTGCTGATCCCCGAGCTGATCA |
| | | AGAGCACCAGCGAGCAGAACACCCTGCTGGAACAGTTCCAACTGAAGGCCGGCGTGCGCAAGCGGT |
| | | ACTCCGACTACAAGAAGCTGCCTACCAAGGAGGTGCTGGCCGAGATCTACAATCCTACTGTGAACAA |
| | | AACCGTCAGCCAGGCCTTCAAGGTGATGGACGCTCTGCTGGAGAAGTACGGCAAGGACCAGATCCA |
| | | CTACATCACCGTGGAAATGCCTAGAGATGATAACGAGGAGGAAGAGCGTAAAAGAATTAAGGAACT |
| | | GCAAACGAAGAATTCTCAGAGAAAGAACGATAGCCAGCAATACTTCCTGCAGAAATCCGGCTGGAG |
| | | CCAGGAGAAGTTCCAGGCCACAATCCACAAGAATAGAAGATTCCTGGCCAAGCTGCTGTACTATTTT |
| | | GAGCAGGACGGCGTGTGTGCCTACACCGGGAACCCCATCAGCCCTGAGCTTCTGGTGTCCGACAGCA |
| | | CCGAGATCGACCACATCATCCCTATCAGCATCAGCCTGGATGACAGCATCAACAACAAAGTGCTGGT |
| | | GCTGTCTCACGCCAACCAGGTGAAGGGCCAACAGACCCCTTACGATGCCAGAATGGCCGGCGCCTTC |
| | | AACAAGATCAATGGAAAGTTCAGCAACTGGGACGAGTATCAAAAGTGGGTGGAAAGCAGACCTTTC |
| | | AGCCGGAAAAAGGTGAACAACCTCCTGGAGACCAGAAACATTTTCGACAGCGAGCAGGTGCAAAAG |
| | | TTTCTGAGCAGAAACCTGAATGATACAAGATACGCCAGCAGACTGGTCCTGAATACCCTGCAGAGCT |
| | | TTTTCGAGAACCAGGATACCATTGTGCGGGTGGTCAACGGCAGCTTCACCCATACCCTGAGAAAGAA |
| | | GTGGGGCGCCGATCTGGACAAAACCCGGGAGACACACCACCACCACGCCGTGGACGCCACACTGTG |
| | | CGCGGTGACACCCTTCGTGAAGGTCTCTAGATACCACTACGCCGTGAACGAGGAGACCGGCGAGAA |
| | | ATTCATGAGAGAAATCGATGTGGAGACCGGCGAAATCCTGGACGAAATTCCATACAGAGAGTACAA |
| | | GAAGGCCAAGCACTACGAGAGAAAAACCTACCAGGTGAAGTGGTCCAACTTCCGCGAGCAGCTTAA |
| | | GCCCATCACAATCCATCCTAAGATCAAGTTCAGCCACCAGGTTGACAGAAAAGCCAACAGAAAGCT |
| | | GAGCGACGCTACCATCTACTCCGTTAGAGAGAAGACAGAGGTGAAAACACTGAAGAGCGGCAAGGA |
| | | GAAGATCACGACCGACGAATACACCATCGGCAAGATCAAGGACATCTATACCGTAGACGGCTGGGA |
| | | AGCCTTTAAAAAGAAGCAGGACAAACTGCTCATGAAGGAATTCGATGAAAAGACCTACGAGCTGCT |
| | | GGTGACCATCGCCGCCACCACCCCTGATTTTCAGGAGGTGGAAGAAAAGAACGGCAAAGTGAAGAG |
| | | AGTGAAGCGGAGCCCTTTCGCCGTGTACTGTGAGGAAAACGGCATACCCGCCATCCGGAAATACGCC |
| | | AAGAAGAACAACGGCCCCGTGATCCGGAGCCTGAAGTACTACGACGGCAAGCTGAATAAGCACATC |
| | | AACATCACCAAGGATGAGAAGGGCAGGCCCGTGGAGCAGACAAAGAACGGCCGGAAAGTGACTCT |
| | | GCAGAGCCTGAAACCCTACCGGTATGATATCTACCAGGACCTCGAAACCAAGGCCTATTATACAGTG |
| | | CAACTGTACTACAGCGACCTGCGCTTTGTGGAGGGCGAGTACGGAATCACAGAAAAAGAGTACATG |
| | | AAGAAGGTGGCCGAACAGACCAAAGGACAGGTGGTGCGGTTCTGCTTCAGCCTGCAGAAGAACGAC |
| | | GGCCTCGAAATCGAGTGGAAGGATTCTCAACGGTACGACGTGAGATTCTACAATTTCCAATCTGCTA |
| | | ACAGCATCAACTTCAAGGGCTTAGAACAGGAGATGATCCCTGCTGAAAACCAGTTCAAGCAGAAGC |
| | | CTTACAACAATGGCGCCATCAACCTGAACATCGCCAAGTATGGCAAGGAGGGCAAGAAACTGAGAA |
| | | AGTTCAACACCGATATCCTGGGTAAAAAGCACCACCTGTCTTATGAGAAAGAGCCGAAGAACATCAT |
| | | CAAGAGCGGCGGAAGCAAAAGACCTGCCGCTACAAAGAAGGCCGGCCAGGCCAAGAAAAAGAAGT |
| | | AAGCTAGCTTGACTGACTGAGATACAGCGTACCTTCAGCTCACAGACATGACTGTGCCTTCTAGTTG |
| | | CCAGCCATCTGTTGTTTGCCCCTCCCCCGTGCCTTCCTTGACCCTGGAAGGTGCCACTCCCACTGTCCT |
| | | TTCCTAATAAAATGAGGAAATTGCATCGCATTGTCTGAGTAGGTGTCATTCTATTCTGGGGGGTGGG |
| | | GTGGGGCAGGACAGCAAGGGGGAGGATTGGGAAGACAATAGCAGGCATGCTGGGGATGCGGTGGG |
| | | CTCTATGGTTTTAAAGCAAGTAAAAGCTTGGTAGGAACCCCTAGTGATGGAGTTGGCCACTCCCTCT |
| | | CTGCGCGCTCGCTCGCTCACTGAGGCCGGGCGACCAAAGGTCGCCCGACGCCCGGGCTTTGCCCGGG |
| | | CGGCCTCAGTGAGCGAGCGAGCGCGCAGAGGCATTTAATTAAGCAAGCTGTCATAGCTGTTTCCTGT |
| | | GTGAAATTGTTATCCGCTCACAATTCCACACAACATACGAGCCGGAAGCATAAAGTGTAAAGCCTGG |
| | | GGTGCCTAATGAGTGAGCTAACTCACATTAATTGCGTTGCGCTCACTGCCCGCTTTCCAGTCGGGAA |
| | | ACCTGTCGTGCCAGCTGCATTAATGAATCGGCCAACGCGCGGGGAGAGGCGGTTTGCGTATTGGGCG |
| | | CTCTTCCGCTTCCTCGCTCACTGACTCGCTGCGCTCGGTCGTTCGGCTGCGGCGAGCGGTATCAGCTC |
| | | ACTCAAAGGCGGTAATACGGTTATCCACAGAATCAGGGGATAACGCAGGAAAGAACATGTGAGCAA |
| | | AAGGCCAGCAAAAGGCCAGGAACCGTAAAAAGGCCGCGTTGCTGGCGTTTTTCCATAGGCTCCGCCC |
| | | CCCTGACGAGCATCACAAAAATCGACGCTCAAGTCAGAGGTGGCGAAACCCGACAGGACTATAAAG |
| | | ATACCAGGCGTTTCCCCCTGGAAGCTCCCTCGTGCGCTCTCCTGTTCCGACCCTGCCGCTTACCGGAT |
| | | ACCTGTCCGCCTTTCTCCCTTCGGGAAGCGTGGCGCTTTCTCATAGCTCACGCTGTAGGTATCTCAGT |
| | | TCGGTGTAGGTCGTTCGCTCCAAGCTGGGCTGTGTGCACGAACCCCCCGTTCAGCCCGACCGCTGCG |
| | | CCTTATCCGGTAACTATCGTCTTGAGTCCAACCCGGTAAGACACGACTTATCGCCACTGGCAGCAGC |
| | | CACTGGTAACAGGATTAGCAGAGCGAGGTATGTAGGCGGTGCTACAGAGTTCTTGAAGTGGTGGCCT |
| | | AACTACGGCTACACTAGAAGAACAGTATTTGGTATCTGCGCTCTGCTGAAGCCAGTTACCTTCGGAA |
| | | AAAGAGTTGGTAGCTCTTGATCCGGCAAACAAACCACCGCTGGTAGCGGTGGTTTTTTTGTTTGCAA |
| | | GCAGCAGATTACGCGCAGAAAAAAAGGATCTCAAGAAGATCCTTTGATCTTTTCTACGGGGTCTGAC |
| | | GCTCAGTGGAACGAAAACTCACGTTAAGGGATTTTGGTCATGAGATTATCAAAAAGGATCTTCACCT |
| | | AGATCCTTTTAAATTAAAAATGAAGTTTTAAATCAAGCCCAATCTGAATAATGTTACAACCAATTAA |
| | | CCAATTCTGATTAGAAAAACTCATCGAGCATCAAATGAAACTGCAATTTATTCATATCAGGATTATC |
| | | AATACCATATTTTTGAAAAAGCCGTTTCTGTAATGAAGGAGAAAACTCACCGAGGCAGTTCCATAGG |
| | | ATGGCAAGATCCTGGTATCGGTCTGCGATTCCGACTCGTCCAACATCAATACAACCTATTAATTTCCC |
| | | CTCGTCAAAAATAAGGTTATCAAGTGAGAAATCACCATGAGTGACGACTGAATCCGGTGAGAATGG |
| | | CAAAAGTTTATGCATTTCTTTCCAGACTTGTTCAACAGGCCAGCCATTACGCTCGTCATCAAAATCAC |
| | | TCGCATCAACCAAACCGTTATTCATTCGTGATTGCGCCTGAGCGAGACGAAATACGCGATCGCTGTT |
| | | AAAAGGACAATTACAAACAGGAATCGAATGCAACCGGCGCAGGAACACTGCCAGCGCATCAACAAT |
| | | ATTTTCACCTGAATCAGGATATTCTTCTAATACCTGGAATGCTGTTTTTCCGGGGATCGCAGTGGTGA |
| | | GTAACCATGCATCATCAGGAGTACGGATAAAATGCTTGATGGTCGGAAGAGGCATAAATTCCGTCAG |
| | | CCAGTTTAGTCTGACCATCTCATCTGTAACATCATTGGCAACGCTACCTTTGCCATGTTTCAGAAACA |
| | | ACTCTGGCGCATCGGGCTTCCCATACAAGCGATAGATTGTCGCACCTGATTGCCCGACATTATCGCG |
| | | AGCCCATTTATACCCATATAAATCAGCATCCATGTTGGAATTTAATCGCGGCCTCGACGTTTCCCGTT |
| | | GAATATGGCTCATAACACCCCTTGTATTACTGTTTATGTAAGCAGACAGTTTTATTGTTCATGATGAT |
| | | ATATTTTTATCTTGTGCAATGTAACATCAGAGATTTTGAGACACGGGCCAGAGCTGCATCGCGCGTTT |
| | | CGGTGATGACGGTGAAAACCTCTGACACATGCAGCTCCCGGAGACGGTCACAGCTTGTCTGTAAGCG |
| | | GATGCCGGGAGCAGACAAGCCCGTCAGGGCGCGTCAGCGGGTGTTGGCGGGTGTCGGGGCTGGCTT |
| | | AACTATGCGGCATCAGAGCAGATTGTACTGAGAGTGCACGATATCGGGTCC |
| 40 | Off-target sequence | CCCAGCCTCCAGGATGCAGTGTTCA |
| | 4282-1 | |
| 41 | Off-target sequence | TGATGCAAGCTACCACCAGG |
| | 4282-1 left primer | |
| 42 | Off-target sequence | TTCAGGAAGCTGAGCCACTG |
| | 4282-1 right primer | |
| 43 | Off-target sequence | TCTGTCATCCAGGTTGAAGTGCACA |
| | 4282-2 | |
| 44 | Off-target sequence | CATGGCAAGTCCCCGTCTCT |
| | 4282-2 left primer | |
| 45 | Off-target sequence | TCACTCTCACACCCAAGATTCCA |
| | 4282-2 right primer | |
| 46 | Off-target sequence | CCCGTCCCCCAGGCTGAAGTGCAA |
| | 4282-3 | |
| 47 | Off-target sequence | GTCCTAGCTACTGGGGCAGC |
| | 4282-3 left primer | |
| 48 | Off-target sequence | GTCCCAGGGGTACACCAATGT |
| | 4282-3 right primer | |
| 49 | Off-target sequence | TCTGTCCTCCAGGCTGAAGTGCAA |
| | 4282-4 | |
| 50 | Off-target sequence | GGACAGGAGCTGGTCGTTGA |
| | 4282-4 left primer | |
| 51 | Off-target sequence | ATGTAGCTGGGCATGGTGGT |
| | 4282-4 right primer | |
| 52 | Off-target sequence | TCTGTCCTCCAGGCTGAAGTGCAA |
| | 4282-5 | |
| 53 | Off-target sequence | TTGCTTACCTTCCCCACTGG |
| | 4282-5 left primer | |
| 54 | Off-target sequence | AGCTAGGCGTGGTGGC |
| | 4282-5 right primer | |
| 55 | Off-target sequence | TCCGTCGTCCAGGATGGAGTGCAA |
| | 4282-6 | |
| 56 | Off-target sequence | CCACGCATGGCCTCCATTTC |
| | 4282-6 left primer | |
| 57 | Off-target sequence | ATGGAAATTAGGGCCGGGCG |
| | 4282-6 right primer | |
| 58 | Off-target sequence | ACAGGCCCCCGGTATGAAGTGCAGCA |
| | 4282-7 | |
| 59 | Off-target sequence | CCGAAATCAGGAAGGCCCTT |
| | 4282-7 left primer | |
| 60 | Off-target sequence | CTTCTATGGTGGCACCGGTT |
| | 4282-7 right primer | |
| 61 | Off-target sequence | TCTGTCCTCCAGGATGAAGTGCAA |
| | 4282-8 | |
| 62 | Off-target sequence | CGTGCTCTGTTCCATCCCTT |
| | 4282-8 left primer | |
| 63 | Off-target sequence | AGTCCCAGCTACTCAGGAGG |
| | 4282-8 right primer | |
| 64 | Off-target sequence | ACTCTGCCTCCAGGCTGAAGTGCAG |
| | 4282-9 | |
| 65 | Off-target sequence | TAGCTGGGCGTGGTTGCA |
| | 4282-9 left primer | |
| 66 | Off-target sequence | CACTGTGCTCCCAACTTGCC |
| | 4282-9 right primer | |
| 67 | APG07433.1 | CATTACAGGCGCTGCTCATA |
| | genomic DNA | |
| | forward primer | |
| 68 | APG07433.1 | TTGCCCTTCTTGTCAATTCC |
| | genomic DNA | |
| | reverse primer | |
| 69 | APG07433.1 | TCCCTTCCGCATGATGGTCTGCT |
| | genomic DNA probe | |
| 70 | mutHTT INDEL left | TCGTCGGCAGCGTCAGATGTGTATAAGAGACAGAAACGAAGGTACACGAGTGG |
| | primer | |
| 71 | mutHTT INDEL | GTCTCGTGGGCTCGGAGATGTGTATAAGAGACAGGCAAACAGGCAGCACAAAAT |
| | right primer | |
| 72 | APG07433.1 mRNA | CATTACAGGCGCTGCTCATA |
| | forward primer | |
| 73 | APG07433.1 mRNA | TTGCCCTTCTTGTCAATTCC |
| | reverse primer | |
| 74 | APG07433.1 probe | TCCCTTCCGCATGATGGTCTGCT |
| 75 | SGN004281 target | ACCGGCCTCCAGGATGAAGTGCACA |
| | sequence | |
| 76 | SGN004282 target | ACCGGCCTCCAGGATGAAGTGCACA |
| | sequence | |
| 77 | SGN002908 target | GAGTTTGTGACCCACGCCTGCTCCC |
| | sequence | |
| 78 | SGN002911 target | TTTGTGACCCACGCCTGCTCCC |
| | sequence | |
| 79 | SGN004333 target | CGGCCTCCAGGATGAAGTGCACACA |
| | sequence | |
| 80 | SGN004281 spacer | ACCGGCCUCCAGGAUGAAGUGCACA |
| | sequence | |
| 81 | SGN004282 spacer | ACCGGCCUCCAGGAUGAAGUGCACA |
| | sequence | |
| 82 | SGN002908 spacer | GAGUUUGUGACCCACGCCUGCUCCC |
| | sequence | |
| 83 | SGN002911 spacer | UUUGUGACCCACGCCUGCUCCC |
| | sequence | |
| 84 | SGN004333 spacer | CGGCCUCCAGGAUGAAGUGCACACA |
| | sequence | |
| 85 | Homo sapiens HTT | MATLEKLMKAFESLKSFQQQQQQQQQQQQQQQQQQQQQPPPPPPPPPPPQLPQPPPQAQP |
| | protein | LLPQPQPPPPPPPPPPGPAVAEEPLHRPKKELSATKKDRVNHCLTICENIVAQSVRNSPE |
| | (NP_001375421.1) | FQKLLGIAMELFLLCSDDAESDVRMVADECLNKVIKALMDSNLPRLQLELYKEIKKNGAP |
| | | RSLRAALWRFAELAHLVRPQKCRPYLVNLLPCLTRTSKRPEESVQETLAAAVPKIMASFG |
| | | NFANDNEIKVLLKAFIANLKSSSPTIRRTAAGSAVSICQHSRRTQYFYSWLLNVLLGLLV |
| | | PVEDEHSTLLILGVLLTLRYLVPLLQQQVKDTSLKGSFGVTRKEMEVSPSAEQLVQVYEL |
| | | TLHHTQHQDHNVVTGALELLQQLFRTPPPELLQTLTAVGGIGQLTAAKEESGGRSRSGSI |
| | | VELIAGGGSSCSPVLSRKQKGKVLLGEEEALEDDSESRSDVSSSALTASVKDEISGELAA |
| | | SSGVSTPGSAGHDIITEQPRSQHTLQADSVDLASCDLTSSATDGDEEDILSHSSSQVSAV |
| | | PSDPAMDLNDGTQASSPISDSSQTTTEGPDSAVTPSDSSEIVLDGTDNQYLGLQIGQPQD |
| | | EDEEATGILPDEASEAFRNSSMALQQAHLLKNMSHCRQPSDSSVDKFVLRDEATEPGDQE |
| | | NKPCRIKGDIGQSTDDDSAPLVHCVRLLSASFLLTGGKNVLVPDRDVRVSVKALALSCVG |
| | | AAVALHPESFFSKLYKVPLDTTEYPEEQYVSDILNYIDHGDPQVRGATAILCGTLICSIL |
| | | SRSRFHVGDWMGTIRTLTGNTFSLADCIPLLRKTLKDESSVTCKLACTAVRNCVMSLCSS |
| | | SYSELGLQLIIDVLTLRNSSYWLVRTELLETLAEIDFRLVSFLEAKAENLHRGAHHYTGL |
| | | LKLQERVLNNVVIHLLGDEDPRVRHVAAASLIRLVPKLFYKCDQGQADPVVAVARDQSSV |
| | | YLKLLMHETQPPSHFSVSTITRIYRGYNLLPSITDVTMENNLSRVIAAVSHELITSTTRA |
| | | LTFGCCEALCLLSTAFPVCIWSLGWHCGVPPLSASDESRKSCTVGMATMILTLLSSAWFP |
| | | LDLSAHQDALILAGNLLAASAPKSLRSSWASEEEANPAATKQEEVWPALGDRALVPMVEQ |
| | | LFSHLLKVINICAHVLDDVAPGPAIKAALPSLTNPPSLSPIRRKGKEKEPGEQASVPLSP |
| | | KKGSEASAASRQSDTSGPVTTSKSSSLGSFYHLPSYLKLHDVLKATHANYKVTLDLQNST |
| | | EKFGGFLRSALDVLSQILELATLQDIGKCVEEILGYLKSCFSREPMMATVCVQQLLKTLF |
| | | GTNLASQFDGLSSNPSKSQGRAQRLGSSSVRPGLYHYCFMAPYTHFTQALADASLRNMVQ |
| | | AEQENDTSGWFDVLQKVSTQLKTNLTSVTKNRADKNAIHNHIRLFEPLVIKALKQYTTTT |
| | | CVQLQKQVLDLLAQLVQLRVNYCLLDSDQVFIGFVLKQFEYIEVGQFRESEAIIPNIFFF |
| | | LVLLSYERYHSKQIIGIPKIIQLCDGIMASGRKAVTHAIPALQPIVHDLFVLRGTNKADA |
| | | GKELETQKEVVVSMLLRLIQYHQVLEMFILVLQQCHKENEDKWKRLSRQIADIILPMLAK |
| | | QQMHIDSHEALGVLNTLFEILAPSSLRPVDMLLRSMFVTPNTMASVSTVQLWISGILAIL |
| | | RVLISQSTEDIVLSRIQELSFSPYLISCTVINRLRDGDSTSTLEEHSEGKQIKNLPEETF |
| | | SRFLLQLVGILLEDIVTKQLKVEMSEQQHTFYCQELGTLLMCLIHIFKSGMFRRITAAAT |
| | | RLFRSDGCGGSFYTLDSLNLRARSMITTHPALVLLWCQILLLVNHTDYRWWAEVQQTPKR |
| | | HSLSSTKLLSPQMSGEEEDSDLAAKLGMCNREIVRRGALILFCDYVCQNLHDSEHLTWLI |
| | | VNHIQDLISLSHEPPVQDFISAVHRNSAASGLFIQAIQSRCENLSTPTMLKKTLQCLEGI |
| | | HLSQSGAVLTLYVDRLLCTPFRVLARMVDILACRRVEMLLAANLQSSMAQLPMEELNRIQ |
| | | EYLQSSGLAQRHQRLYSLLDRFRLSTMQDSLSPSPPVSSHPLDGDGHVSLETVSPDKDWY |
| | | VHLVKSQCWTRSDSALLEGAELVNRIPAEDMNAFMMNSEFNLSLLAPCLSLGMSEISGGQ |
| | | KSALFEAAREVTLARVSGTVQQLPAVHHVFQPELPAEPAAYWSKLNDLFGDAALYQSLPT |
| | | LARALAQYLVVVSKLPSHLHLPPEKEKDIVKFVVATLEALSWHLIHEQIPLSLDLQAGLD |
| | | CCCLALQLPGLWSVVSSTEFVTHACSLIYCVHFILEAVAVQPGEQLLSPERRTNTPKAIS |
| | | EEEEEVDPNTQNPKYITAACEMVAEMVESLQSVLALGHKRNSGVPAFLTPLLRNIIISLA |
| | | RLPLVNSYTRVPPLVWKLGWSPKPGGDFGTAFPEIPVEFLQEKEVFKEFIYRINTLGWTS |
| | | RTQFEETWATLLGVLVTQPLVMEQEESPPEEDTERTQINVLAVQAITSLVLSAMTVPVAG |
| | | NPAVSCLEQQPRNKPLKALDTRFGRKLSIIRGIVEQEIQAMVSKRENIATHHLYQAWDPV |
| | | PSLSPATTGALISHEKLLLQINPERELGSMSYKLGQVSIHSVWLGNSITPLREEEWDEEE |
| | | EEEADAPAPSSPPTSPVNSRKHRAGVDIHSCSQFLLELYSRWILPSSSARRTPAILISEV |
| | | VRSLLVVSDLFTERNQFELMYVTLTELRRVHPSEDEILAQYLVPATCKAAAVLGMDKAVA |
| | | EPVSRLLESTLRSSHLPSRVGALHGVLYVLECDLLDDTAKQLIPVISDYLLSNLKGIAHC |
| | | VNIHSQQHVLVMCATAFYLIENYPLDVGPEFSASIIQMCGVMLSGSEESTPSIIYHCALR |
| | | GLERLLLSEQLSRLDAESLVKLSVDRVNVHSPHRAMAALGLMLTCMYTGKEKVSPGRTSD |
| | | PNPAAPDSESVIVAMERVSVLFDRIRKGFPCEARVVARILPQFLDDFFPPQDIMNKVIGE |
| | | FLSNQQPYPQFMATVVYKVFQTLHSTGQSSMVRDWVMLSLSNFTQRAPVAMATWSLSCFF |
| | | VSASTSPWVAAILPHVISRMGKLEQVDVNLFCLVATDFYRHQIEEELDRRAFQSVLEVVA |
| | | APGSPYHRLLTCLRNVHKVTTC |
| 86 | SV40 nuclear | PKKKRKV |
| | localization signal | |
| 87 | nucleoplasmin | KRPAATKKAGQAKKKK |
| | nuclear localization | |
| | signal | |
| 88 | Mammalian codon- | ATGTACTCTATCGGCCTCGATCTGGGCATCTCTAGCGTCGGCTGGTCGGTGATCGACGAAGAGACAG |
| | optimized APG05586 | GCAAAATCGTTGACCTGGGCGTGCGGCTGTTTTCTGCTAAGAACAGCGAGAAGAATCTGGAGAGAA |
| | | GAACCAGCAGAGGCGCAAGAAGACTGATCAGAAGAAAGACGAACCGACTAAAAGACGCCAAGAAA |
| | | TTACTGGAGGCCATCGGCTTCTACGAAGATAAGGCCCTTAAAAATGTCTGCCCTTACCAACTGAGAG |
| | | TGAAGGGCCTGACCGAGGGCCTGACAAAGGGCGAACTGTACAAGGTCGTGCTGCACATCGTGAAGA |
| | | AACGGGGCATCAGCTACCTGGACGAGGACGACGCCGAAGCTGCCAAAGAATCTCAGGACTACAAGG |
| | | AGCAGGTACGCAAAAATGCCCAACTGCTGACAAAGTACACCCCTGGACAGATCCAACTGCAGAGAC |
| | | TGAAGGAGAACAACAGAGTGAAGACAGGAATCAACGGCCAGGGCCACTACCAACTGAACGTGTTCA |
| | | AGGTGAGCGCTTACGCTGACGAGCTGGCCACCATCCTGAAGACCCAGCAAGCCCTGTATCCTAACGA |
| | | ACTGACCGACGACTGGATCGCCCTGTTCGTGCAACCTGGAATTGCCGAGAATGCCGGCCTTATCTAC |
| | | AGAAAGAGGCCTTACTACCACGGCCCTGGCAACGAGGCCAACAACAGCCCTTACGGAAGATGGTCC |
| | | GACTTCCAGAAAACAGGCCAGCCAGCCGCTAACATCTTCGACAAGCTAATCGGAAAGGATTTCCAG |
| | | GGCGAGCTGCGGGCCAGCGGCCTGAGCCTGTCTGCCCAGCAATACAACCTGCTGAACGACCTGACCA |
| | | ATCTGAAAATTGACGGAGAGGTGTCCCTGTCCCCCGAGCAGAAGGAATTCATCCTGACCGAGCTGAT |
| | | GACCAAGGAATTCGCCAGATTCGGCGTCAACGACATTGCCAAGCTGCTGGGCGTGAAAAAAGAACA |
| | | GTTGTCTGGCTGGCGGCTGGACAAGAAGGGCAAGCCCGAAATCCACACCCTGAAGGGCTACAGAAA |
| | | CTGGAGAAAGATCTTTGCCGAGGCCGGAATCGACCTGGCCACACTGCCAACAGAGACCATTGACTGC |
| | | CTGGCCAAGGTTCTGACCCTGAACACCGAGCGGGAAGGAGTGGAAAACACCCTGGCCTTTGAGCTG |
| | | CCTGAACTGGCTGAGCCTGTGAAGAGCCTGGTGTTGGACCATTACAAGGAACTGAGCCAAAGCATCT |
| | | CAACACAGGCTTGGCACAGATTCAGCCTGAAGACACTGCACCTGCTGATCCCCGAGCTGATCAAGAG |
| | | CACCAGCGAGCAGAACACCCTGCTGGAACAGTTCCAACTGAAGGCCGGCGTGCGCAAGCGGTACTC |
| | | CGACTACAAGAAGCTGCCTACCAAGGAGGTGCTGGCCGAGATCTACAATCCTACTGTGAACAAAAC |
| | | CGTCAGCCAGGCCTTCAAGGTGATGGACGCTCTGCTGGAGAAGTACGGCAAGGACCAGATCCACTA |
| | | CATCACCGTGGAAATGCCTAGAGATGATAACGAGGAGGAAGAGCGTAAAAGAATTAAGGAACTGCA |
| | | AACGAAGAATTCTCAGAGAAAGAACGATAGCCAGCAATACTTCCTGCAGAAATCCGGCTGGAGCCA |
| | | GGAGAAGTTCCAGGCCACAATCCACAAGAATAGAAGATTCCTGGCCAAGCTGCTGTACTATTTTGAG |
| | | CAGGACGGCGTGTGTGCCTACACCGGGAACCCCATCAGCCCTGAGCTTCTGGTGTCCGACAGCACCG |
| | | AGATCGACCACATCATCCCTATCAGCATCAGCCTGGATGACAGCATCAACAACAAAGTGCTGGTGCT |
| | | GTCTCACGCCAACCAGGTGAAGGGCCAACAGACCCCTTACGATGCCAGAATGGCCGGCGCCTTCAAC |
| | | AAGATCAATGGAAAGTTCAGCAACTGGGACGAGTATCAAAAGTGGGTGGAAAGCAGACCTTTCAGC |
| | | CGGAAAAAGGTGAACAACCTCCTGGAGACCAGAAACATTTTCGACAGCGAGCAGGTGCAAAAGTTT |
| | | CTGAGCAGAAACCTGAATGATACAAGATACGCCAGCAGACTGGTCCTGAATACCCTGCAGAGCTTTT |
| | | TCGAGAACCAGGATACCATTGTGCGGGTGGTCAACGGCAGCTTCACCCATACCCTGAGAAAGAAGT |
| | | GGGGCGCCGATCTGGACAAAACCCGGGAGACACACCACCACCACGCCGTGGACGCCACACTGTGCG |
| | | CGGTGACACCCTTCGTGAAGGTCTCTAGATACCACTACGCCGTGAACGAGGAGACCGGCGAGAAATT |
| | | CATGAGAGAAATCGATGTGGAGACCGGCGAAATCCTGGACGAAATTCCATACAGAGAGTACAAGAA |
| | | GGCCAAGCACTACGAGAGAAAAACCTACCAGGTGAAGTGGTCCAACTTCCGCGAGCAGCTTAAGCC |
| | | CATCACAATCCATCCTAAGATCAAGTTCAGCCACCAGGTTGACAGAAAAGCCAACAGAAAGCTGAG |
| | | CGACGCTACCATCTACTCCGTTAGAGAGAAGACAGAGGTGAAAACACTGAAGAGCGGCAAGGAGAA |
| | | GATCACGACCGACGAATACACCATCGGCAAGATCAAGGACATCTATACCGTAGACGGCTGGGAAGC |
| | | CTTTAAAAAGAAGCAGGACAAACTGCTCATGAAGGAATTCGATGAAAAGACCTACGAGCTGCTGGT |
| | | GACCATCGCCGCCACCACCCCTGATTTTCAGGAGGTGGAAGAAAAGAACGGCAAAGTGAAGAGAGT |
| | | GAAGCGGAGCCCTTTCGCCGTGTACTGTGAGGAAAACGGCATACCCGCCATCCGGAAATACGCCAA |
| | | GAAGAACAACGGCCCCGTGATCCGGAGCCTGAAGTACTACGACGGCAAGCTGAATAAGCACATCAA |
| | | CATCACCAAGGATGAGAAGGGCAGGCCCGTGGAGCAGACAAAGAACGGCCGGAAAGTGACTCTGC |
| | | AGAGCCTGAAACCCTACCGGTATGATATCTACCAGGACCTCGAAACCAAGGCCTATTATACAGTGCA |
| | | ACTGTACTACAGCGACCTGCGCTTTGTGGAGGGCGAGTACGGAATCACAGAAAAAGAGTACATGAA |
| | | GAAGGTGGCCGAACAGACCAAAGGACAGGTGGTGCGGTTCTGCTTCAGCCTGCAGAAGAACGACGG |
| | | CCTCGAAATCGAGTGGAAGGATTCTCAACGGTACGACGTGAGATTCTACAATTTCCAATCTGCTAAC |
| | | AGCATCAACTTCAAGGGCTTAGAACAGGAGATGATCCCTGCTGAAAACCAGTTCAAGCAGAAGCCTT |
| | | ACAACAATGGCGCCATCAACCTGAACATCGCCAAGTATGGCAAGGAGGGCAAGAAACTGAGAAAGT |
| | | TCAACACCGATATCCTGGGTAAAAAGCACCACCTGTCTTATGAGAAAGAGCCGAAGAACATCATCA |
| | | AG |
| 89 | truncated U6 | TGTACAAAAAAGCAGGCTTTAAAGGAACCAATTCAGTCGACTGGATCCGGTACCAAGGTCGGGCAG |
| | promoter | GAAGAGGGCCTATTTCCCATGATTCCTTCATATTTGCATATACGATACAAGGCTGTTAGAGAGATAA |
| | (hU6(318 bp) | TTAGAATTAATTTGACTGTAAACACAAAGATATTAGTACAAAATACGTGACGTAGAAAGTAATAATT |
| | promoter) | TCTTGGGTAGTTTGCAGTTTTAAAATTATGTTTTAAAATGGACTATCATATGCTTACCGTAACTTGAA |
| | | AGTATTTCGATTTCTTGGCTTTATATATCTTGTGGAAAGGACGAAACACC |
| 90 | CMVeb promoter | GTGATGCGGTTTTGGCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGACTTTCCAAAATGTCGT |
| | | AACAACTCCGCCCCATTGACGCAAATGGGCGGTAGGCGTGTACGGTGGGAGGTCTATATAAGCAGA |
| | | GCTCGTTTAGTGAACCGTCAGATC |
| 91 | EFS promoter | GAATTCGCTAGCTAGGTCTTGAAAGGAGTGGGAATTGGCTCCGGTGCCCGTCAGTGGGCAGAGCGCA |
| | | CATCGCCCACAGTCCCCGAGAAGTTGGGGGGAGGGGTCGGCAATTGATCCGGTGCCTAGAGAAGGT |
| | | GGCGCGGGGTAAACTGGGAAAGTGATGTCGTGTACTGGCTCCGCCTTTTTCCCGAGGGTGGGGGAGA |
| | | ACCGTATATAAGTGCAGTAGTCGCCGTGAACGTTCTTTTTCGCAACGGGTTTGCCGCCAGAACACAG |
| | | GACCGGTGCCACC |
| 92 | JeT promoter | GAATTCGGGCGGAGTTAGGGCGGAGCCAATCAGCGTGCGCCGTTCCGAAAGTTGCCTTTTATGGCTG |
| | | GGCGGAGAATGGGCGGTGAACGCCGATGATTATATAAGGACGCGCCGGGTGTGGCACAGCTAGTTC |
| | | CGTCGCAGCCGGGATTTGGGTCGCGGTTCTTGTTTGTGGATCCCTGTGATCGTCACTTGACA |
| 93 | Synapsin promoter | AGTGCAAGTGGGTTTTAGGACCAGGATGAGGCGGGGTGGGGGTGCCTACCTGACGACCGACCCCGA |
| | | CCCACTGGACAAGCACCCAACCCCCATTCCCCAAATTGCGCATCCCCTATCAGAGAGGGGGAGGGG |
| | | AAACAGGATGCGGCGAGGCGCGTGCGCACTGCCAGCTTCAGCACCGCGGACAGTGCCTTCGCCCCC |
| | | GCCTGGCGGCGCGCGCCACCGCCGCCTCAGCACTGAAGGCGCGCTGACGTCACTCGCCGGTCCCCCG |
| | | CAAACTCCCCTTCCCGGCCACCTTGGTCGCGTCCGCGCCGCCGCCGGCCCAGCCGGACCGCACCACG |
| | | CGAGGCGCGAGATAGGGGGGCACGGGCGCGACCATCTGCGCTGCGGCGCCGGCGACTCAGCGCTGC |
| | | CTCAGTCTGCGGTGGGCAGCGGAGGAGTCGTGTCGTGCCTGAGAGCGCAG |
| 94 | SV40 polyA | TAAGATACATTGATGAGTTTGGACAAACCACAACTAGAATGCAGTGAAAAAAATGCTTTATTTGTGA |
| | | AATTTGTGATGCTATTGCTTTATTTGTAACCATTATAAGCTGCAATAAACAAGTTAACAACAACAATT |
| | | GCATTCATTTTATGTTTCAGGTTCAGGGGGAGGTGTGGGAGGTT |
| 95 | bGH polyA | CTGTGCCTTCTAGTTGCCAGCCATCTGTTGTTTGCCCCTCCCCCGTGCCTTCCTTGACCCTGGAAGGTG |
| | | CCACTCCCACTGTCCTTTCCTAATAAAATGAGGAAATTGCATCGCATTGTCTGAGTAGGTGTCATTCT |
| | | ATTCTGGGGGGTGGGGTGGGGCAGGACAGCAAGGGGGAGGATTGGGAAGACAATAGCAGGCATGC |
| | | TGGGGATGCGGTGGGCTCTATGG |
| 96 | NC_041760 RNA pol | AGCTCCACGGCAAGAGAATTCAAAGCCGCGGGCCTGGGTTCCACGCGGGGCCCCTTACCCAAGGTG |
| | III promoter | CCCCGGGCGCTCATTTGCATGTCCCACCCAACAGGTAAACCTGACAGGTCAGTCGCGGCCGGGTACG |
| | | GCCTGGCGGTCAGAGCACCAAACGTACGAGCCTTGTGATGCGGTTCCATTGCATGAAATTCTCCTAA |
| | | AGGCCCCAAGATGAACGGGAAAGCGCGCGGTTCGCTCACCGTAACTAAAACAGGTGAGAGACTCCC |
| | | GTGCCTTATAAGGCCTGTGGACGGAGGCAGTTGC |
| 97 | NW_0048481 RNA | TGTTCTCCGAACAGTACTTGTAATATACAGGTTCCCCGATCCACCACCGTCTGCGGGTGGCGGTACA |
| | pol III promoter | GCCTCTCCCAGTGTGCTTTGCGCTCATTTGCATAGAGCACGGCACCGAACGCAGCCACTGTCCCCCGC |
| | | GTCCGCTGTCCCCCGTGCAGGCCAAACCCGGGCGCGGCCCCGCCGACGGTGGGTACAGAGAGTGGT |
| | | CGGCTCGGCACCGGCTGCTGCGCGGCACCGGCACTCACCCTGTCCTTATGGAGCCCTAACTCCATGG |
| | | CTATAAATATCTAAGGGGAGGAAGGGTAGATC |
| 98 | LR738627 RNA pol | AGCGCTCCGCGGAGACTTCTGGGACGCGGCGGCTCCGACTCCGCCCCGCTTCCGGCTTATTTGCATA |
| | III promoter | CAGCAATTCCTAGCAGGCCCTGTGCTGAATTTAGTCGGAAAACTACCATGTTCAGTCGAAAAAGCAA |
| | | ATACTTTTGTCAGATATGGCCAAAAACTTCACTTGACTTAGCCGTGTTTCATGTAAAGCATTAAAAGG |
| | | ATGGAGTGATTGTTCAAATTTCATAAGAAGAATTCACCTTCAGTTTAAGGTGGTTCGCTTTCTGCACT |
| | | TCAAATACCGCGGTGGACAGACCCTGTTTC |
| 99 | NC_044556 RNA pol | GCCTGAGTCGCGCCGCCGCCTCCCAAAGACTTCTGGGAAGGCGGTGCGGCTCAGGCTCCGCCCCGCT |
| | III promoter | TCCGGGGATATTTGCATACGAGCATTCCCAGTAATTCCCAGCAGCCACTGTAGCTATATTTGGTAGA |
| | | ATAACGAGCACTTTCTGAACTCCAGTTAATAACTGCGTTAGTTGCGTTACACATTGGACTAAAACAA |
| | | ATAGAAGTTGAATCTCTAGAGCAGTGGAGATAAGTCGCCGTATGTGTACAGAAATTGCTTCCGGGGG |
| | | CTATAAATAGCTGGTAGTGGGGCTAGAACGTC |
| 100 | XM 0308455 RNA | CGCCCGCGCAGCGCAGCCGCTTCCCAGAGACTTCTGGGACGGCAGCGGCTGCGGCTCCGCCCCTCTT |
| | pol III promoter | CCAGTATAATTTGCATGCGACCATGGATTCCCAGCAGCCACCTGAGTCATATTTGGTGGAACAAAAA |
| | | CCACTTTCTCAATTTCAGTGAATGACCCCATTTGGTTAAGGTATTGTTGCACAAATATCATAAAAGAA |
| | | GGGACGAATGGCTGAACCGGTTTTTAATGGAGTTCGCCCTTAGCGTAAAAGAGTTTATTCTATGCCCT |
| | | CTAAATAGTTCTGGGATCAACCGTACTACT |
| 101 | Mini NC_041760 | CCCCTTACCCAAGGTGCCCCGGGCGCTCATTTGCATGTCCCACCGCTCACCGTAACTAAAACAGGTG |
| | RNA pol III promoter | AGAGACTCCCGTGCCTTATAAGGCCTGTGGACGGAGGCAGTTGC |
| 102 | Mini NW_0048481 | ACAGCCTCTCCCAGTGTGCTTTGCGCTCATTTGCATAGAGCACGACTCACCCTGTCCTTATGGAGCCC |
| | RNA pol III promoter | TAACTCCATGGCTATAAATATCTAAGGGGAGGAAGGGTAGATC |
| 103 | Mini LR738627 | GGCTCCGACTCCGCCCCGCTTCCGGCTTATTTGCATACAGCAATATTCACCTTCAGTTTAAGGTGGTT |
| | RNA pol III promoter | CGCTTTCTGCACTTCAAATACCGCGGTGGACAGACCCTGTTTC |
| 104 | Mini NC_044556 | GCTCAGGCTCCGCCCCGCTTCCGGGGATATTTGCATACGAGCATAGTCGCCGTATGTGTACAGAAAT |
| | RNA pol III promoter | TGCTTCCGGGGGCTATAAATAGCTGGTAGTGGGGCTAGAACGTC |
| 105 | Mini XM_0308455 | GCTGCGGCTCCGCCCCTCTTCCAGTATAATTTGCATGCGACCATGTTCGCCCTTAGCGTAAAAGAGTT |
| | RNA pol III promoter | TATTCTATGCCCTCTAAATAGTTCTGGGATCAACCGTACTACT |
| 106 | APG05586 truncated | GUUAUUGUACUC |
| | crRNA repeat | |
| 107 | APG05586 truncated | GAAUCUACAAUAAUAAGGCAUCUUGCCGAAUUUACCGCCCUACAUAUGUAGGGCGGUUUU |
| | tracrRNA | |
| 108 | APG05586 genomic | GGCTACCATCCTGAAAACTC |
| | DNA forward primer | |
| 109 | APG05586 genomic | GGTCCATGATAATAAGGGCG |
| | DNA reverse primer | |
| 110 | APG05586 genomic | TGCGTTATTTGTGCAACCAGGT |
| | DNA probe | |
| 111 | APG05586 mRNA | GGCTACCATCCTGAAAACTC |
| | forward primer | |
| 112 | APG05586 mRNA | GGTCCATGATAATAAGGGCG |
| | reverse primer | |
| 113 | APG05586 probe | TGCGTTATTTGTGCAACCAGGT |
| 114 | APG05586mco | GCCGAGATCTACAATCCTAC |
| | genomic DNA forward | |
| | primer | |
| 115 | APG05586mco | GTGATGTAGTGGATCTGGTC |
| | genomic DNA reverse | |
| | primer | |
| 116 | APG05586mco | CCTTCAAGGTGATGGACGCT |
| | genomic DNA probe | |
| 117 | APG05586mco mRNA | GCCGAGATCTACAATCCTAC |
| | forward primer | |
| 118 | APG05586mco mRNA | GTGATGTAGTGGATCTGGTC |
| | reverse primer | |
| 119 | APG05586mco probe | CCTTCAAGGTGATGGACGCT |
| 120 | APG01604 tracrRNA | UUACAGAAUCUACUAAAACAAGACUAUAUGUCGUGUUUAUCCCAUCAAUUUAUUGAUGGGAUUU |
| | (85 backbone) | |
| 121 | pAAV-hU6(318 bp)- | GTGCACGATATCGGGTCCCCAATTGCTGCGCGCTCGCTCGCTCACTGAGGCCGCCCGGGCAAAGCCC |
| | SGN004282- | GGGCGTCGGGCGACCTTTGGTCGCCCGGCCTCAGTGAGCGAGCGAGCGCGCAGAGAGGGAGTGGCC |
| | CMVeb-SV40-NLS- | AACTCCATCACTAGGGGTTCCTCGTCGTGTACAAAAAAGCAGGCTTTAAAGGAACCAATTCAGTCGA |
| | APG05586mco-NP- | CTGGATCCGGTACCAAGGTCGGGCAGGAAGAGGGCCTATTTCCCATGATTCCTTCATATTTGCATAT |
| | NLS- | ACGATACAAGGCTGTTAGAGAGATAATTAGAATTAATTTGACTGTAAACACAAAGATATTAGTACAA |
| | SV40pA(179 bp)- | AATACGTGACGTAGAAAGTAATAATTTCTTGGGTAGTTTGCAGTTTTAAAATTATGTTTTAAAATGGA |
| | MINIMIZED | CTATCATATGCTTACCGTAACTTGAAAGTATTTCGATTTCTTGGCTTTATATATCTTGTGGAAAGGAC |
| | | GAAACACCGACCGGCCTCCAGGATGAAGTGCACAGTTATTGTACTCAAAGGAATCTACAATAATAA |
| | | GGCATCTTGCCGAATTTACCGCCCTACATATGTAGGGCGGTTTTTTTTTGTGATATCCTCGTGATGCG |
| | | GTTTTGGCAGTACATCAATGGGCGTGGATAGCGGTTTGACTCACGGGGATTTCCAAGTCTCCACCCC |
| | | ATTGACGTCAATGGGAGTTTGTTTTGGCACCAAAATCAACGGGACTTTCCAAAATGTCGTAACAACT |
| | | CCGCCCCATTGACGCAAATGGGCGGTAGGCGTGTACGGTGGGAGGTCTATATAAGCAGAGCTCGTTT |
| | | AGTGAACCGTCAGATCGCCTCTTTGTCGAGCCACCATGGCCCCTAAGAAGAAAAGAAAGGTGATGTA |
| | | CTCTATCGGCCTCGATCTGGGCATCTCTAGCGTCGGCTGGTCGGTGATCGACGAAGAGACAGGCAAA |
| | | ATCGTTGACCTGGGCGTGCGGCTGTTTTCTGCTAAGAACAGCGAGAAGAATCTGGAGAGAAGAACC |
| | | AGCAGAGGCGCAAGAAGACTGATCAGAAGAAAGACGAACCGACTAAAAGACGCCAAGAAATTACT |
| | | GGAGGCCATCGGCTTCTACGAAGATAAGGCCCTTAAAAATGTCTGCCCTTACCAACTGAGAGTGAAG |
| | | GGCCTGACCGAGGGCCTGACAAAGGGCGAACTGTACAAGGTCGTGCTGCACATCGTGAAGAAACGG |
| | | GGCATCAGCTACCTGGACGAGGACGACGCCGAAGCTGCCAAAGAATCTCAGGACTACAAGGAGCAG |
| | | GTACGCAAAAATGCCCAACTGCTGACAAAGTACACCCCTGGACAGATCCAACTGCAGAGACTGAAG |
| | | GAGAACAACAGAGTGAAGACAGGAATCAACGGCCAGGGCCACTACCAACTGAACGTGTTCAAGGTG |
| | | AGCGCTTACGCTGACGAGCTGGCCACCATCCTGAAGACCCAGCAAGCCCTGTATCCTAACGAACTGA |
| | | CCGACGACTGGATCGCCCTGTTCGTGCAACCTGGAATTGCCGAGAATGCCGGCCTTATCTACAGAAA |
| | | GAGGCCTTACTACCACGGCCCTGGCAACGAGGCCAACAACAGCCCTTACGGAAGATGGTCCGACTTC |
| | | CAGAAAACAGGCCAGCCAGCCGCTAACATCTTCGACAAGCTAATCGGAAAGGATTTCCAGGGCGAG |
| | | CTGCGGGCCAGCGGCCTGAGCCTGTCTGCCCAGCAATACAACCTGCTGAACGACCTGACCAATCTGA |
| | | AAATTGACGGAGAGGTGTCCCTGTCCCCCGAGCAGAAGGAATTCATCCTGACCGAGCTGATGACCAA |
| | | GGAATTCGCCAGATTCGGCGTCAACGACATTGCCAAGCTGCTGGGCGTGAAAAAAGAACAGTTGTCT |
| | | GGCTGGCGGCTGGACAAGAAGGGCAAGCCCGAAATCCACACCCTGAAGGGCTACAGAAACTGGAGA |
| | | AAGATCTTTGCCGAGGCCGGAATCGACCTGGCCACACTGCCAACAGAGACCATTGACTGCCTGGCCA |
| | | AGGTTCTGACCCTGAACACCGAGCGGGAAGGAGTGGAAAACACCCTGGCCTTTGAGCTGCCTGAACT |
| | | GGCTGAGCCTGTGAAGAGCCTGGTGTTGGACCATTACAAGGAACTGAGCCAAAGCATCTCAACACA |
| | | GGCTTGGCACAGATTCAGCCTGAAGACACTGCACCTGCTGATCCCCGAGCTGATCAAGAGCACCAGC |
| | | GAGCAGAACACCCTGCTGGAACAGTTCCAACTGAAGGCCGGCGTGCGCAAGCGGTACTCCGACTAC |
| | | AAGAAGCTGCCTACCAAGGAGGTGCTGGCCGAGATCTACAATCCTACTGTGAACAAAACCGTCAGC |
| | | CAGGCCTTCAAGGTGATGGACGCTCTGCTGGAGAAGTACGGCAAGGACCAGATCCACTACATCACC |
| | | GTGGAAATGCCTAGAGATGATAACGAGGAGGAAGAGCGTAAAAGAATTAAGGAACTGCAAACGAA |
| | | GAATTCTCAGAGAAAGAACGATAGCCAGCAATACTTCCTGCAGAAATCCGGCTGGAGCCAGGAGAA |
| | | GTTCCAGGCCACAATCCACAAGAATAGAAGATTCCTGGCCAAGCTGCTGTACTATTTTGAGCAGGAC |
| | | GGCGTGTGTGCCTACACCGGGAACCCCATCAGCCCTGAGCTTCTGGTGTCCGACAGCACCGAGATCG |
| | | ACCACATCATCCCTATCAGCATCAGCCTGGATGACAGCATCAACAACAAAGTGCTGGTGCTGTCTCA |
| | | CGCCAACCAGGTGAAGGGCCAACAGACCCCTTACGATGCCAGAATGGCCGGCGCCTTCAACAAGAT |
| | | CAATGGAAAGTTCAGCAACTGGGACGAGTATCAAAAGTGGGTGGAAAGCAGACCTTTCAGCCGGAA |
| | | AAAGGTGAACAACCTCCTGGAGACCAGAAACATTTTCGACAGCGAGCAGGTGCAAAAGTTTCTGAG |
| | | CAGAAACCTGAATGATACAAGATACGCCAGCAGACTGGTCCTGAATACCCTGCAGAGCTTTTTCGAG |
| | | AACCAGGATACCATTGTGCGGGTGGTCAACGGCAGCTTCACCCATACCCTGAGAAAGAAGTGGGGC |
| | | GCCGATCTGGACAAAACCCGGGAGACACACCACCACCACGCCGTGGACGCCACACTGTGCGCGGTG |
| | | ACACCCTTCGTGAAGGTCTCTAGATACCACTACGCCGTGAACGAGGAGACCGGCGAGAAATTCATGA |
| | | GAGAAATCGATGTGGAGACCGGCGAAATCCTGGACGAAATTCCATACAGAGAGTACAAGAAGGCCA |
| | | AGCACTACGAGAGAAAAACCTACCAGGTGAAGTGGTCCAACTTCCGCGAGCAGCTTAAGCCCATCA |
| | | CAATCCATCCTAAGATCAAGTTCAGCCACCAGGTTGACAGAAAAGCCAACAGAAAGCTGAGCGACG |
| | | CTACCATCTACTCCGTTAGAGAGAAGACAGAGGTGAAAACACTGAAGAGCGGCAAGGAGAAGATCA |
| | | CGACCGACGAATACACCATCGGCAAGATCAAGGACATCTATACCGTAGACGGCTGGGAAGCCTTTA |
| | | AAAAGAAGCAGGACAAACTGCTCATGAAGGAATTCGATGAAAAGACCTACGAGCTGCTGGTGACCA |
| | | TCGCCGCCACCACCCCTGATTTTCAGGAGGTGGAAGAAAAGAACGGCAAAGTGAAGAGAGTGAAGC |
| | | GGAGCCCTTTCGCCGTGTACTGTGAGGAAAACGGCATACCCGCCATCCGGAAATACGCCAAGAAGA |
| | | ACAACGGCCCCGTGATCCGGAGCCTGAAGTACTACGACGGCAAGCTGAATAAGCACATCAACATCA |
| | | CCAAGGATGAGAAGGGCAGGCCCGTGGAGCAGACAAAGAACGGCCGGAAAGTGACTCTGCAGAGC |
| | | CTGAAACCCTACCGGTATGATATCTACCAGGACCTCGAAACCAAGGCCTATTATACAGTGCAACTGT |
| | | ACTACAGCGACCTGCGCTTTGTGGAGGGCGAGTACGGAATCACAGAAAAAGAGTACATGAAGAAGG |
| | | TGGCCGAACAGACCAAAGGACAGGTGGTGCGGTTCTGCTTCAGCCTGCAGAAGAACGACGGCCTCG |
| | | AAATCGAGTGGAAGGATTCTCAACGGTACGACGTGAGATTCTACAATTTCCAATCTGCTAACAGCAT |
| | | CAACTTCAAGGGCTTAGAACAGGAGATGATCCCTGCTGAAAACCAGTTCAAGCAGAAGCCTTACAA |
| | | CAATGGCGCCATCAACCTGAACATCGCCAAGTATGGCAAGGAGGGCAAGAAACTGAGAAAGTTCAA |
| | | CACCGATATCCTGGGTAAAAAGCACCACCTGTCTTATGAGAAAGAGCCGAAGAACATCATCAAGAG |
| | | CGGCGGAAGCAAAAGACCTGCCGCTACAAAGAAGGCCGGCCAGGCCAAGAAAAAGAAGTAATGAT |
| | | AAGATACATTGATGAGTTTGGACAAACCACAACTAGAATGCAGTGAAAAAAATGCTTTATTTGTGAA |
| | | ATTTGTGATGCTATTGCTTTATTTGTAACCATTATAAGCTGCAATAAACAAGTTAACAACAACAATTG |
| | | CATTCATTTTATGTTTCAGGTTCAGGGGGAGGTGTGGGAGGTTTTTTAAAGGAACCCCTAGTGATGG |
| | | AGTTGGCCACTCCCTCTCTGCGCGCTCGCTCGCTCACTGAGGCCGGGCGACCAAAGGTCGCCCGACG |
| | | CCCGGGCTTTGCCCGGGCGGCCTCAGTGAGCGAGCGAGCGCGCAGAGGCATTTAATTAAGCAAGCT |
| | | GTCATAGCTGTTTCCTGTGTGAAATTGTTATCCGCTCACAATTCCACACAACATACGAGCCGGAAGC |
| | | ATAAAGTGTAAAGCCTGGGGTGCCTAATGAGTGAGCTAACTCACATTAATTGCGTTGCGCTCACTGC |
| | | CCGCTTTCCAGTCGGGAAACCTGTCGTGCCAGCTGCATTAATGAATCGGCCAACGCGCGGGGAGAGG |
| | | CGGTTTGCGTATTGGGCGCTCTTCCGCTTCCTCGCTCACTGACTCGCTGCGCTCGGTCGTTCGGCTGC |
| | | GGCGAGCGGTATCAGCTCACTCAAAGGCGGTAATACGGTTATCCACAGAATCAGGGGATAACGCAG |
| | | GAAAGAACATGTGAGCAAAAGGCCAGCAAAAGGCCAGGAACCGTAAAAAGGCCGCGTTGCTGGCG |
| | | TTTTTCCATAGGCTCCGCCCCCCTGACGAGCATCACAAAAATCGACGCTCAAGTCAGAGGTGGCGAA |
| | | ACCCGACAGGACTATAAAGATACCAGGCGTTTCCCCCTGGAAGCTCCCTCGTGCGCTCTCCTGTTCCG |
| | | ACCCTGCCGCTTACCGGATACCTGTCCGCCTTTCTCCCTTCGGGAAGCGTGGCGCTTTCTCATAGCTC |
| | | ACGCTGTAGGTATCTCAGTTCGGTGTAGGTCGTTCGCTCCAAGCTGGGCTGTGTGCACGAACCCCCC |
| | | GTTCAGCCCGACCGCTGCGCCTTATCCGGTAACTATCGTCTTGAGTCCAACCCGGTAAGACACGACTT |
| | | ATCGCCACTGGCAGCAGCCACTGGTAACAGGATTAGCAGAGCGAGGTATGTAGGCGGTGCTACAGA |
| | | GTTCTTGAAGTGGTGGCCTAACTACGGCTACACTAGAAGAACAGTATTTGGTATCTGCGCTCTGCTG |
| | | AAGCCAGTTACCTTCGGAAAAAGAGTTGGTAGCTCTTGATCCGGCAAACAAACCACCGCTGGTAGCG |
| | | GTGGTTTTTTTGTTTGCAAGCAGCAGATTACGCGCAGAAAAAAAGGATCTCAAGAAGATCCTTTGAT |
| | | CTTTTCTACGGGGTCTGACGCTCAGTGGAACGAAAACTCACGTTAAGGGATTTTGGTCATGAGATTA |
| | | TCAAAAAGGATCTTCACCTAGATCCTTTTAAATTAAAAATGAAGTTTTAAATCAAGCCCAATCTGAA |
| | | TAATGTTACAACCAATTAACCAATTCTGATTAGAAAAACTCATCGAGCATCAAATGAAACTGCAATT |
| | | TATTCATATCAGGATTATCAATACCATATTTTTGAAAAAGCCGTTTCTGTAATGAAGGAGAAAACTC |
| | | ACCGAGGCAGTTCCATAGGATGGCAAGATCCTGGTATCGGTCTGCGATTCCGACTCGTCCAACATCA |
| | | ATACAACCTATTAATTTCCCCTCGTCAAAAATAAGGTTATCAAGTGAGAAATCACCATGAGTGACGA |
| | | CTGAATCCGGTGAGAATGGCAAAAGTTTATGCATTTCTTTCCAGACTTGTTCAACAGGCCAGCCATTA |
| | | CGCTCGTCATCAAAATCACTCGCATCAACCAAACCGTTATTCATTCGTGATTGCGCCTGAGCGAGAC |
| | | GAAATACGCGATCGCTGTTAAAAGGACAATTACAAACAGGAATCGAATGCAACCGGCGCAGGAACA |
| | | CTGCCAGCGCATCAACAATATTTTCACCTGAATCAGGATATTCTTCTAATACCTGGAATGCTGTTTTT |
| | | CCGGGGATCGCAGTGGTGAGTAACCATGCATCATCAGGAGTACGGATAAAATGCTTGATGGTCGGA |
| | | AGAGGCATAAATTCCGTCAGCCAGTTTAGTCTGACCATCTCATCTGTAACATCATTGGCAACGCTACC |
| | | TTTGCCATGTTTCAGAAACAACTCTGGCGCATCGGGCTTCCCATACAAGCGATAGATTGTCGCACCTG |
| | | ATTGCCCGACATTATCGCGAGCCCATTTATACCCATATAAATCAGCATCCATGTTGGAATTTAATCGC |
| | | GGCCTCGACGTTTCCCGTTGAATATGGCTCATAACACCCCTTGTATTACTGTTTATGTAAGCAGACAG |
| | | TTTTATTGTTCATGATGATATATTTTTATCTTGTGCAATGTAACATCAGAGATTTTGAGACACGGGCC |
| | | AGAGCTGCATCGCGCGTTTCGGTGATGACGGTGAAAACCTCTGACACATGCAGCTCCCGGAGACGGT |
| | | CACAGCTTGTCTGTAAGCGGATGCCGGGAGCAGACAAGCCCGTCAGGGCGCGTCAGCGGGTGTTGG |
| | | CGGGTGTCGGGGCTGGCTTAACTATGCGGCATCAGAGCAGATTGTACTGAGAGTGCACGATATCGGG |
| | | TCC |
| 122 | pAAV-hU6(249 bp)- | GTGCACGATATCGGGTCCCCAATTGCTGCGCGCTCGCTCGCTCACTGAGGCCGCCCGGGCAAAGCCC |
| | SGN004282- | GGGCGTCGGGCGACCTTTGGTCGCCCGGCCTCAGTGAGCGAGCGAGCGCGCAGAGAGGGAGTGGCC |
| | CMVeb-SV40-NLS- | AACTCCATCACTAGGGGTTCCTCGTCGGAGGGCCTATTTCCCATGATTCCTTCATATTTGCATATACG |
| | APG05586mco-NP- | ATACAAGGCTGTTAGAGAGATAATTGGAATTAATTTGACTGTAAACACAAAGATATTAGTACAAAAT |
| | NLS- | ACGTGACGTAGAAAGTAATAATTTCTTGGGTAGTTTGCAGTTTTAAAATTATGTTTTAAAATGGACTA |
| | SV40pA(179 bp) | TCATATGCTTACCGTAACTTGAAAGTATTTCGATTTCTTGGCTTTATATATCTTGTGGAAAGGACGAA |
| | | ACACCGACCGGCCTCCAGGATGAAGTGCACAGTTATTGTACTCAAAGGAATCTACAATAATAAGGCA |
| | | TCTTGCCGAATTTACCGCCCTACATATGTAGGGCGGTTTTTTTTTGTGATATCCTCGTGATGCGGTTTT |
| | | GGCAGTACATCAATGGGCGTGGATAGCGGTTTGACTCACGGGGATTTCCAAGTCTCCACCCCATTGA |
| | | CGTCAATGGGAGTTTGTTTTGGCACCAAAATCAACGGGACTTTCCAAAATGTCGTAACAACTCCGCC |
| | | CCATTGACGCAAATGGGCGGTAGGCGTGTACGGTGGGAGGTCTATATAAGCAGAGCTCGTTTAGTGA |
| | | ACCGTCAGATCGCCTCTTTGTCGAGCCACCATGGCCCCTAAGAAGAAAAGAAAGGTGATGTACTCTA |
| | | TCGGCCTCGATCTGGGCATCTCTAGCGTCGGCTGGTCGGTGATCGACGAAGAGACAGGCAAAATCGT |
| | | TGACCTGGGCGTGCGGCTGTTTTCTGCTAAGAACAGCGAGAAGAATCTGGAGAGAAGAACCAGCAG |
| | | AGGCGCAAGAAGACTGATCAGAAGAAAGACGAACCGACTAAAAGACGCCAAGAAATTACTGGAGG |
| | | CCATCGGCTTCTACGAAGATAAGGCCCTTAAAAATGTCTGCCCTTACCAACTGAGAGTGAAGGGCCT |
| | | GACCGAGGGCCTGACAAAGGGCGAACTGTACAAGGTCGTGCTGCACATCGTGAAGAAACGGGGCAT |
| | | CAGCTACCTGGACGAGGACGACGCCGAAGCTGCCAAAGAATCTCAGGACTACAAGGAGCAGGTACG |
| | | CAAAAATGCCCAACTGCTGACAAAGTACACCCCTGGACAGATCCAACTGCAGAGACTGAAGGAGAA |
| | | CAACAGAGTGAAGACAGGAATCAACGGCCAGGGCCACTACCAACTGAACGTGTTCAAGGTGAGCGC |
| | | TTACGCTGACGAGCTGGCCACCATCCTGAAGACCCAGCAAGCCCTGTATCCTAACGAACTGACCGAC |
| | | GACTGGATCGCCCTGTTCGTGCAACCTGGAATTGCCGAGAATGCCGGCCTTATCTACAGAAAGAGGC |
| | | CTTACTACCACGGCCCTGGCAACGAGGCCAACAACAGCCCTTACGGAAGATGGTCCGACTTCCAGAA |
| | | AACAGGCCAGCCAGCCGCTAACATCTTCGACAAGCTAATCGGAAAGGATTTCCAGGGCGAGCTGCG |
| | | GGCCAGCGGCCTGAGCCTGTCTGCCCAGCAATACAACCTGCTGAACGACCTGACCAATCTGAAAATT |
| | | GACGGAGAGGTGTCCCTGTCCCCCGAGCAGAAGGAATTCATCCTGACCGAGCTGATGACCAAGGAA |
| | | TTCGCCAGATTCGGCGTCAACGACATTGCCAAGCTGCTGGGCGTGAAAAAAGAACAGTTGTCTGGCT |
| | | GGCGGCTGGACAAGAAGGGCAAGCCCGAAATCCACACCCTGAAGGGCTACAGAAACTGGAGAAAG |
| | | ATCTTTGCCGAGGCCGGAATCGACCTGGCCACACTGCCAACAGAGACCATTGACTGCCTGGCCAAGG |
| | | TTCTGACCCTGAACACCGAGCGGGAAGGAGTGGAAAACACCCTGGCCTTTGAGCTGCCTGAACTGGC |
| | | TGAGCCTGTGAAGAGCCTGGTGTTGGACCATTACAAGGAACTGAGCCAAAGCATCTCAACACAGGCT |
| | | TGGCACAGATTCAGCCTGAAGACACTGCACCTGCTGATCCCCGAGCTGATCAAGAGCACCAGCGAGC |
| | | AGAACACCCTGCTGGAACAGTTCCAACTGAAGGCCGGCGTGCGCAAGCGGTACTCCGACTACAAGA |
| | | AGCTGCCTACCAAGGAGGTGCTGGCCGAGATCTACAATCCTACTGTGAACAAAACCGTCAGCCAGGC |
| | | CTTCAAGGTGATGGACGCTCTGCTGGAGAAGTACGGCAAGGACCAGATCCACTACATCACCGTGGA |
| | | AATGCCTAGAGATGATAACGAGGAGGAAGAGCGTAAAAGAATTAAGGAACTGCAAACGAAGAATT |
| | | CTCAGAGAAAGAACGATAGCCAGCAATACTTCCTGCAGAAATCCGGCTGGAGCCAGGAGAAGTTCC |
| | | AGGCCACAATCCACAAGAATAGAAGATTCCTGGCCAAGCTGCTGTACTATTTTGAGCAGGACGGCGT |
| | | GTGTGCCTACACCGGGAACCCCATCAGCCCTGAGCTTCTGGTGTCCGACAGCACCGAGATCGACCAC |
| | | ATCATCCCTATCAGCATCAGCCTGGATGACAGCATCAACAACAAAGTGCTGGTGCTGTCTCACGCCA |
| | | ACCAGGTGAAGGGCCAACAGACCCCTTACGATGCCAGAATGGCCGGCGCCTTCAACAAGATCAATG |
| | | GAAAGTTCAGCAACTGGGACGAGTATCAAAAGTGGGTGGAAAGCAGACCTTTCAGCCGGAAAAAGG |
| | | TGAACAACCTCCTGGAGACCAGAAACATTTTCGACAGCGAGCAGGTGCAAAAGTTTCTGAGCAGAA |
| | | ACCTGAATGATACAAGATACGCCAGCAGACTGGTCCTGAATACCCTGCAGAGCTTTTTCGAGAACCA |
| | | GGATACCATTGTGCGGGTGGTCAACGGCAGCTTCACCCATACCCTGAGAAAGAAGTGGGGCGCCGA |
| | | TCTGGACAAAACCCGGGAGACACACCACCACCACGCCGTGGACGCCACACTGTGCGCGGTGACACC |
| | | CTTCGTGAAGGTCTCTAGATACCACTACGCCGTGAACGAGGAGACCGGCGAGAAATTCATGAGAGA |
| | | AATCGATGTGGAGACCGGCGAAATCCTGGACGAAATTCCATACAGAGAGTACAAGAAGGCCAAGCA |
| | | CTACGAGAGAAAAACCTACCAGGTGAAGTGGTCCAACTTCCGCGAGCAGCTTAAGCCCATCACAATC |
| | | CATCCTAAGATCAAGTTCAGCCACCAGGTTGACAGAAAAGCCAACAGAAAGCTGAGCGACGCTACC |
| | | ATCTACTCCGTTAGAGAGAAGACAGAGGTGAAAACACTGAAGAGCGGCAAGGAGAAGATCACGACC |
| | | GACGAATACACCATCGGCAAGATCAAGGACATCTATACCGTAGACGGCTGGGAAGCCTTTAAAAAG |
| | | AAGCAGGACAAACTGCTCATGAAGGAATTCGATGAAAAGACCTACGAGCTGCTGGTGACCATCGCC |
| | | GCCACCACCCCTGATTTTCAGGAGGTGGAAGAAAAGAACGGCAAAGTGAAGAGAGTGAAGCGGAGC |
| | | CCTTTCGCCGTGTACTGTGAGGAAAACGGCATACCCGCCATCCGGAAATACGCCAAGAAGAACAAC |
| | | GGCCCCGTGATCCGGAGCCTGAAGTACTACGACGGCAAGCTGAATAAGCACATCAACATCACCAAG |
| | | GATGAGAAGGGCAGGCCCGTGGAGCAGACAAAGAACGGCCGGAAAGTGACTCTGCAGAGCCTGAA |
| | | ACCCTACCGGTATGATATCTACCAGGACCTCGAAACCAAGGCCTATTATACAGTGCAACTGTACTAC |
| | | AGCGACCTGCGCTTTGTGGAGGGCGAGTACGGAATCACAGAAAAAGAGTACATGAAGAAGGTGGCC |
| | | GAACAGACCAAAGGACAGGTGGTGCGGTTCTGCTTCAGCCTGCAGAAGAACGACGGCCTCGAAATC |
| | | GAGTGGAAGGATTCTCAACGGTACGACGTGAGATTCTACAATTTCCAATCTGCTAACAGCATCAACT |
| | | TCAAGGGCTTAGAACAGGAGATGATCCCTGCTGAAAACCAGTTCAAGCAGAAGCCTTACAACAATG |
| | | GCGCCATCAACCTGAACATCGCCAAGTATGGCAAGGAGGGCAAGAAACTGAGAAAGTTCAACACCG |
| | | ATATCCTGGGTAAAAAGCACCACCTGTCTTATGAGAAAGAGCCGAAGAACATCATCAAGAGCGGCG |
| | | GAAGCAAAAGACCTGCCGCTACAAAGAAGGCCGGCCAGGCCAAGAAAAAGAAGTAATGATAAGAT |
| | | ACATTGATGAGTTTGGACAAACCACAACTAGAATGCAGTGAAAAAAATGCTTTATTTGTGAAATTTG |
| | | TGATGCTATTGCTTTATTTGTAACCATTATAAGCTGCAATAAACAAGTTAACAACAACAATTGCATTC |
| | | ATTTTATGTTTCAGGTTCAGGGGGAGGTGTGGGAGGTTTTTTAAAGGAACCCCTAGTGATGGAGTTG |
| | | GCCACTCCCTCTCTGCGCGCTCGCTCGCTCACTGAGGCCGGGCGACCAAAGGTCGCCCGACGCCCGG |
| | | GCTTTGCCCGGGCGGCCTCAGTGAGCGAGCGAGCGCGCAGAGGCATTTAATTAAGCAAGCTGTCATA |
| | | GCTGTTTCCTGTGTGAAATTGTTATCCGCTCACAATTCCACACAACATACGAGCCGGAAGCATAAAG |
| | | TGTAAAGCCTGGGGTGCCTAATGAGTGAGCTAACTCACATTAATTGCGTTGCGCTCACTGCCCGCTTT |
| | | CCAGTCGGGAAACCTGTCGTGCCAGCTGCATTAATGAATCGGCCAACGCGCGGGGAGAGGCGGTTT |
| | | GCGTATTGGGCGCTCTTCCGCTTCCTCGCTCACTGACTCGCTGCGCTCGGTCGTTCGGCTGCGGCGAG |
| | | CGGTATCAGCTCACTCAAAGGCGGTAATACGGTTATCCACAGAATCAGGGGATAACGCAGGAAAGA |
| | | ACATGTGAGCAAAAGGCCAGCAAAAGGCCAGGAACCGTAAAAAGGCCGCGTTGCTGGCGTTTTTCC |
| | | ATAGGCTCCGCCCCCCTGACGAGCATCACAAAAATCGACGCTCAAGTCAGAGGTGGCGAAACCCGA |
| | | CAGGACTATAAAGATACCAGGCGTTTCCCCCTGGAAGCTCCCTCGTGCGCTCTCCTGTTCCGACCCTG |
| | | CCGCTTACCGGATACCTGTCCGCCTTTCTCCCTTCGGGAAGCGTGGCGCTTTCTCATAGCTCACGCTG |
| | | TAGGTATCTCAGTTCGGTGTAGGTCGTTCGCTCCAAGCTGGGCTGTGTGCACGAACCCCCCGTTCAGC |
| | | CCGACCGCTGCGCCTTATCCGGTAACTATCGTCTTGAGTCCAACCCGGTAAGACACGACTTATCGCC |
| | | ACTGGCAGCAGCCACTGGTAACAGGATTAGCAGAGCGAGGTATGTAGGCGGTGCTACAGAGTTCTT |
| | | GAAGTGGTGGCCTAACTACGGCTACACTAGAAGAACAGTATTTGGTATCTGCGCTCTGCTGAAGCCA |
| | | GTTACCTTCGGAAAAAGAGTTGGTAGCTCTTGATCCGGCAAACAAACCACCGCTGGTAGCGGTGGTT |
| | | TTTTTGTTTGCAAGCAGCAGATTACGCGCAGAAAAAAAGGATCTCAAGAAGATCCTTTGATCTTTTCT |
| | | ACGGGGTCTGACGCTCAGTGGAACGAAAACTCACGTTAAGGGATTTTGGTCATGAGATTATCAAAAA |
| | | GGATCTTCACCTAGATCCTTTTAAATTAAAAATGAAGTTTTAAATCAAGCCCAATCTGAATAATGTTA |
| | | CAACCAATTAACCAATTCTGATTAGAAAAACTCATCGAGCATCAAATGAAACTGCAATTTATTCATA |
| | | TCAGGATTATCAATACCATATTTTTGAAAAAGCCGTTTCTGTAATGAAGGAGAAAACTCACCGAGGC |
| | | AGTTCCATAGGATGGCAAGATCCTGGTATCGGTCTGCGATTCCGACTCGTCCAACATCAATACAACC |
| | | TATTAATTTCCCCTCGTCAAAAATAAGGTTATCAAGTGAGAAATCACCATGAGTGACGACTGAATCC |
| | | GGTGAGAATGGCAAAAGTTTATGCATTTCTTTCCAGACTTGTTCAACAGGCCAGCCATTACGCTCGTC |
| | | ATCAAAATCACTCGCATCAACCAAACCGTTATTCATTCGTGATTGCGCCTGAGCGAGACGAAATACG |
| | | CGATCGCTGTTAAAAGGACAATTACAAACAGGAATCGAATGCAACCGGCGCAGGAACACTGCCAGC |
| | | GCATCAACAATATTTTCACCTGAATCAGGATATTCTTCTAATACCTGGAATGCTGTTTTTCCGGGGAT |
| | | CGCAGTGGTGAGTAACCATGCATCATCAGGAGTACGGATAAAATGCTTGATGGTCGGAAGAGGCAT |
| | | AAATTCCGTCAGCCAGTTTAGTCTGACCATCTCATCTGTAACATCATTGGCAACGCTACCTTTGCCAT |
| | | GTTTCAGAAACAACTCTGGCGCATCGGGCTTCCCATACAAGCGATAGATTGTCGCACCTGATTGCCC |
| | | GACATTATCGCGAGCCCATTTATACCCATATAAATCAGCATCCATGTTGGAATTTAATCGCGGCCTCG |
| | | ACGTTTCCCGTTGAATATGGCTCATAACACCCCTTGTATTACTGTTTATGTAAGCAGACAGTTTTATT |
| | | GTTCATGATGATATATTTTTATCTTGTGCAATGTAACATCAGAGATTTTGAGACACGGGCCAGAGCTG |
| | | CATCGCGCGTTTCGGTGATGACGGTGAAAACCTCTGACACATGCAGCTCCCGGAGACGGTCACAGCT |
| | | TGTCTGTAAGCGGATGCCGGGAGCAGACAAGCCCGTCAGGGCGCGTCAGCGGGTGTTGGCGGGTGT |
| | | CGGGGCTGGCTTAACTATGCGGCATCAGAGCAGATTGTACTGAGAGTGCACGATATCGGGTCC |
| 123 | pAAV-hU6(249 bp)- | GTGCACGATATCGGGTCCCCAATTGCTGCGCGCTCGCTCGCTCACTGAGGCCGCCCGGGCAAAGCCC |
| | SGN004282- | GGGCGTCGGGCGACCTTTGGTCGCCCGGCCTCAGTGAGCGAGCGAGCGCGCAGAGAGGGAGTGGCC |
| | CMVeb-c-MYC- | AACTCCATCACTAGGGGTTCCTCGTCGGAGGGCCTATTTCCCATGATTCCTTCATATTTGCATATACG |
| | NLS-APG05586mco- | ATACAAGGCTGTTAGAGAGATAATTGGAATTAATTTGACTGTAAACACAAAGATATTAGTACAAAAT |
| | c-MYC-NLS- | ACGTGACGTAGAAAGTAATAATTTCTTGGGTAGTTTGCAGTTTTAAAATTATGTTTTAAAATGGACTA |
| | SV40pA(179 bp) | TCATATGCTTACCGTAACTTGAAAGTATTTCGATTTCTTGGCTTTATATATCTTGTGGAAAGGACGAA |
| | | ACACCGACCGGCCTCCAGGATGAAGTGCACAGTTATTGTACTCAAAGGAATCTACAATAATAAGGCA |
| | | TCTTGCCGAATTTACCGCCCTACATATGTAGGGCGGTTTTTTTTTGTGATATCCTCGTGATGCGGTTTT |
| | | GGCAGTACATCAATGGGCGTGGATAGCGGTTTGACTCACGGGGATTTCCAAGTCTCCACCCCATTGA |
| | | CGTCAATGGGAGTTTGTTTTGGCACCAAAATCAACGGGACTTTCCAAAATGTCGTAACAACTCCGCC |
| | | CCATTGACGCAAATGGGCGGTAGGCGTGTACGGTGGGAGGTCTATATAAGCAGAGCTCGTTTAGTGA |
| | | ACCGTCAGATCGCCTCTTTGTCGAGCCACCATGGCCCCTGCTGCCAAGCGCGTGAAGCTGGACGGCA |
| | | GCTCTGGCGGAAGTTCCGGAATGTACTCTATCGGCCTCGATCTGGGCATCTCTAGCGTCGGCTGGTCG |
| | | GTGATCGACGAAGAGACAGGCAAAATCGTTGACCTGGGCGTGCGGCTGTTTTCTGCTAAGAACAGCG |
| | | AGAAGAATCTGGAGAGAAGAACCAGCAGAGGCGCAAGAAGACTGATCAGAAGAAAGACGAACCGA |
| | | CTAAAAGACGCCAAGAAATTACTGGAGGCCATCGGCTTCTACGAAGATAAGGCCCTTAAAAATGTCT |
| | | GCCCTTACCAACTGAGAGTGAAGGGCCTGACCGAGGGCCTGACAAAGGGCGAACTGTACAAGGTCG |
| | | TGCTGCACATCGTGAAGAAACGGGGCATCAGCTACCTGGACGAGGACGACGCCGAAGCTGCCAAAG |
| | | AATCTCAGGACTACAAGGAGCAGGTACGCAAAAATGCCCAACTGCTGACAAAGTACACCCCTGGAC |
| | | AGATCCAACTGCAGAGACTGAAGGAGAACAACAGAGTGAAGACAGGAATCAACGGCCAGGGCCAC |
| | | TACCAACTGAACGTGTTCAAGGTGAGCGCTTACGCTGACGAGCTGGCCACCATCCTGAAGACCCAGC |
| | | AAGCCCTGTATCCTAACGAACTGACCGACGACTGGATCGCCCTGTTCGTGCAACCTGGAATTGCCGA |
| | | GAATGCCGGCCTTATCTACAGAAAGAGGCCTTACTACCACGGCCCTGGCAACGAGGCCAACAACAG |
| | | CCCTTACGGAAGATGGTCCGACTTCCAGAAAACAGGCCAGCCAGCCGCTAACATCTTCGACAAGCTA |
| | | ATCGGAAAGGATTTCCAGGGCGAGCTGCGGGCCAGCGGCCTGAGCCTGTCTGCCCAGCAATACAAC |
| | | CTGCTGAACGACCTGACCAATCTGAAAATTGACGGAGAGGTGTCCCTGTCCCCCGAGCAGAAGGAAT |
| | | TCATCCTGACCGAGCTGATGACCAAGGAATTCGCCAGATTCGGCGTCAACGACATTGCCAAGCTGCT |
| | | GGGCGTGAAAAAAGAACAGTTGTCTGGCTGGCGGCTGGACAAGAAGGGCAAGCCCGAAATCCACAC |
| | | CCTGAAGGGCTACAGAAACTGGAGAAAGATCTTTGCCGAGGCCGGAATCGACCTGGCCACACTGCC |
| | | AACAGAGACCATTGACTGCCTGGCCAAGGTTCTGACCCTGAACACCGAGCGGGAAGGAGTGGAAAA |
| | | CACCCTGGCCTTTGAGCTGCCTGAACTGGCTGAGCCTGTGAAGAGCCTGGTGTTGGACCATTACAAG |
| | | GAACTGAGCCAAAGCATCTCAACACAGGCTTGGCACAGATTCAGCCTGAAGACACTGCACCTGCTGA |
| | | TCCCCGAGCTGATCAAGAGCACCAGCGAGCAGAACACCCTGCTGGAACAGTTCCAACTGAAGGCCG |
| | | GCGTGCGCAAGCGGTACTCCGACTACAAGAAGCTGCCTACCAAGGAGGTGCTGGCCGAGATCTACA |
| | | ATCCTACTGTGAACAAAACCGTCAGCCAGGCCTTCAAGGTGATGGACGCTCTGCTGGAGAAGTACGG |
| | | CAAGGACCAGATCCACTACATCACCGTGGAAATGCCTAGAGATGATAACGAGGAGGAAGAGCGTAA |
| | | AAGAATTAAGGAACTGCAAACGAAGAATTCTCAGAGAAAGAACGATAGCCAGCAATACTTCCTGCA |
| | | GAAATCCGGCTGGAGCCAGGAGAAGTTCCAGGCCACAATCCACAAGAATAGAAGATTCCTGGCCAA |
| | | GCTGCTGTACTATTTTGAGCAGGACGGCGTGTGTGCCTACACCGGGAACCCCATCAGCCCTGAGCTT |
| | | CTGGTGTCCGACAGCACCGAGATCGACCACATCATCCCTATCAGCATCAGCCTGGATGACAGCATCA |
| | | ACAACAAAGTGCTGGTGCTGTCTCACGCCAACCAGGTGAAGGGCCAACAGACCCCTTACGATGCCA |
| | | GAATGGCCGGCGCCTTCAACAAGATCAATGGAAAGTTCAGCAACTGGGACGAGTATCAAAAGTGGG |
| | | TGGAAAGCAGACCTTTCAGCCGGAAAAAGGTGAACAACCTCCTGGAGACCAGAAACATTTTCGACA |
| | | GCGAGCAGGTGCAAAAGTTTCTGAGCAGAAACCTGAATGATACAAGATACGCCAGCAGACTGGTCC |
| | | TGAATACCCTGCAGAGCTTTTTCGAGAACCAGGATACCATTGTGCGGGTGGTCAACGGCAGCTTCAC |
| | | CCATACCCTGAGAAAGAAGTGGGGCGCCGATCTGGACAAAACCCGGGAGACACACCACCACCACGC |
| | | CGTGGACGCCACACTGTGCGCGGTGACACCCTTCGTGAAGGTCTCTAGATACCACTACGCCGTGAAC |
| | | GAGGAGACCGGCGAGAAATTCATGAGAGAAATCGATGTGGAGACCGGCGAAATCCTGGACGAAATT |
| | | CCATACAGAGAGTACAAGAAGGCCAAGCACTACGAGAGAAAAACCTACCAGGTGAAGTGGTCCAAC |
| | | TTCCGCGAGCAGCTTAAGCCCATCACAATCCATCCTAAGATCAAGTTCAGCCACCAGGTTGACAGAA |
| | | AAGCCAACAGAAAGCTGAGCGACGCTACCATCTACTCCGTTAGAGAGAAGACAGAGGTGAAAACAC |
| | | TGAAGAGCGGCAAGGAGAAGATCACGACCGACGAATACACCATCGGCAAGATCAAGGACATCTATA |
| | | CCGTAGACGGCTGGGAAGCCTTTAAAAAGAAGCAGGACAAACTGCTCATGAAGGAATTCGATGAAA |
| | | AGACCTACGAGCTGCTGGTGACCATCGCCGCCACCACCCCTGATTTTCAGGAGGTGGAAGAAAAGA |
| | | ACGGCAAAGTGAAGAGAGTGAAGCGGAGCCCTTTCGCCGTGTACTGTGAGGAAAACGGCATACCCG |
| | | CCATCCGGAAATACGCCAAGAAGAACAACGGCCCCGTGATCCGGAGCCTGAAGTACTACGACGGCA |
| | | AGCTGAATAAGCACATCAACATCACCAAGGATGAGAAGGGCAGGCCCGTGGAGCAGACAAAGAAC |
| | | GGCCGGAAAGTGACTCTGCAGAGCCTGAAACCCTACCGGTATGATATCTACCAGGACCTCGAAACCA |
| | | AGGCCTATTATACAGTGCAACTGTACTACAGCGACCTGCGCTTTGTGGAGGGCGAGTACGGAATCAC |
| | | AGAAAAAGAGTACATGAAGAAGGTGGCCGAACAGACCAAAGGACAGGTGGTGCGGTTCTGCTTCAG |
| | | CCTGCAGAAGAACGACGGCCTCGAAATCGAGTGGAAGGATTCTCAACGGTACGACGTGAGATTCTA |
| | | CAATTTCCAATCTGCTAACAGCATCAACTTCAAGGGCTTAGAACAGGAGATGATCCCTGCTGAAAAC |
| | | CAGTTCAAGCAGAAGCCTTACAACAATGGCGCCATCAACCTGAACATCGCCAAGTATGGCAAGGAG |
| | | GGCAAGAAACTGAGAAAGTTCAACACCGATATCCTGGGTAAAAAGCACCACCTGTCTTATGAGAAA |
| | | GAGCCGAAGAACATCATCAAGGGCAGCTCTGGCGGAAGTTCCGGACCTGCTGCCAAGCGCGTGAAG |
| | | CTGGACTAATGATAAGATACATTGATGAGTTTGGACAAACCACAACTAGAATGCAGTGAAAAAAAT |
| | | GCTTTATTTGTGAAATTTGTGATGCTATTGCTTTATTTGTAACCATTATAAGCTGCAATAAACAAGTT |
| | | AACAACAACAATTGCATTCATTTTATGTTTCAGGTTCAGGGGGAGGTGTGGGAGGTTTTTTAAAGGA |
| | | ACCCCTAGTGATGGAGTTGGCCACTCCCTCTCTGCGCGCTCGCTCGCTCACTGAGGCCGGGCGACCA |
| | | AAGGTCGCCCGACGCCCGGGCTTTGCCCGGGCGGCCTCAGTGAGCGAGCGAGCGCGCAGAGGCATT |
| | | TAATTAAGCAAGCTGTCATAGCTGTTTCCTGTGTGAAATTGTTATCCGCTCACAATTCCACACAACAT |
| | | ACGAGCCGGAAGCATAAAGTGTAAAGCCTGGGGTGCCTAATGAGTGAGCTAACTCACATTAATTGC |
| | | GTTGCGCTCACTGCCCGCTTTCCAGTCGGGAAACCTGTCGTGCCAGCTGCATTAATGAATCGGCCAA |
| | | CGCGCGGGGAGAGGCGGTTTGCGTATTGGGCGCTCTTCCGCTTCCTCGCTCACTGACTCGCTGCGCTC |
| | | GGTCGTTCGGCTGCGGCGAGCGGTATCAGCTCACTCAAAGGCGGTAATACGGTTATCCACAGAATCA |
| | | GGGGATAACGCAGGAAAGAACATGTGAGCAAAAGGCCAGCAAAAGGCCAGGAACCGTAAAAAGGC |
| | | CGCGTTGCTGGCGTTTTTCCATAGGCTCCGCCCCCCTGACGAGCATCACAAAAATCGACGCTCAAGT |
| | | CAGAGGTGGCGAAACCCGACAGGACTATAAAGATACCAGGCGTTTCCCCCTGGAAGCTCCCTCGTGC |
| | | GCTCTCCTGTTCCGACCCTGCCGCTTACCGGATACCTGTCCGCCTTTCTCCCTTCGGGAAGCGTGGCG |
| | | CTTTCTCATAGCTCACGCTGTAGGTATCTCAGTTCGGTGTAGGTCGTTCGCTCCAAGCTGGGCTGTGT |
| | | GCACGAACCCCCCGTTCAGCCCGACCGCTGCGCCTTATCCGGTAACTATCGTCTTGAGTCCAACCCG |
| | | GTAAGACACGACTTATCGCCACTGGCAGCAGCCACTGGTAACAGGATTAGCAGAGCGAGGTATGTA |
| | | GGCGGTGCTACAGAGTTCTTGAAGTGGTGGCCTAACTACGGCTACACTAGAAGAACAGTATTTGGTA |
| | | TCTGCGCTCTGCTGAAGCCAGTTACCTTCGGAAAAAGAGTTGGTAGCTCTTGATCCGGCAAACAAAC |
| | | CACCGCTGGTAGCGGTGGTTTTTTTGTTTGCAAGCAGCAGATTACGCGCAGAAAAAAAGGATCTCAA |
| | | GAAGATCCTTTGATCTTTTCTACGGGGTCTGACGCTCAGTGGAACGAAAACTCACGTTAAGGGATTTT |
| | | GGTCATGAGATTATCAAAAAGGATCTTCACCTAGATCCTTTTAAATTAAAAATGAAGTTTTAAATCA |
| | | AGCCCAATCTGAATAATGTTACAACCAATTAACCAATTCTGATTAGAAAAACTCATCGAGCATCAAA |
| | | TGAAACTGCAATTTATTCATATCAGGATTATCAATACCATATTTTTGAAAAAGCCGTTTCTGTAATGA |
| | | AGGAGAAAACTCACCGAGGCAGTTCCATAGGATGGCAAGATCCTGGTATCGGTCTGCGATTCCGACT |
| | | CGTCCAACATCAATACAACCTATTAATTTCCCCTCGTCAAAAATAAGGTTATCAAGTGAGAAATCAC |
| | | CATGAGTGACGACTGAATCCGGTGAGAATGGCAAAAGTTTATGCATTTCTTTCCAGACTTGTTCAAC |
| | | AGGCCAGCCATTACGCTCGTCATCAAAATCACTCGCATCAACCAAACCGTTATTCATTCGTGATTGCG |
| | | CCTGAGCGAGACGAAATACGCGATCGCTGTTAAAAGGACAATTACAAACAGGAATCGAATGCAACC |
| | | GGCGCAGGAACACTGCCAGCGCATCAACAATATTTTCACCTGAATCAGGATATTCTTCTAATACCTG |
| | | GAATGCTGTTTTTCCGGGGATCGCAGTGGTGAGTAACCATGCATCATCAGGAGTACGGATAAAATGC |
| | | TTGATGGTCGGAAGAGGCATAAATTCCGTCAGCCAGTTTAGTCTGACCATCTCATCTGTAACATCATT |
| | | GGCAACGCTACCTTTGCCATGTTTCAGAAACAACTCTGGCGCATCGGGCTTCCCATACAAGCGATAG |
| | | ATTGTCGCACCTGATTGCCCGACATTATCGCGAGCCCATTTATACCCATATAAATCAGCATCCATGTT |
| | | GGAATTTAATCGCGGCCTCGACGTTTCCCGTTGAATATGGCTCATAACACCCCTTGTATTACTGTTTA |
| | | TGTAAGCAGACAGTTTTATTGTTCATGATGATATATTTTTATCTTGTGCAATGTAACATCAGAGATTT |
| | | TGAGACACGGGCCAGAGCTGCATCGCGCGTTTCGGTGATGACGGTGAAAACCTCTGACACATGCAGC |
| | | TCCCGGAGACGGTCACAGCTTGTCTGTAAGCGGATGCCGGGAGCAGACAAGCCCGTCAGGGCGCGT |
| | | CAGCGGGTGTTGGCGGGTGTCGGGGCTGGCTTAACTATGCGGCATCAGAGCAGATTGTACTGAGAGT |
| | | GCACGATATCGGGTCC |
| 124 | c-MYC NLS | CCTGCTGCCAAGCGCGTGAAGCTGGAC |
| 125 | c-MYC NLS | PAAKRVKLD |
| 126 | NLS linker | GGCAGCTCTGGCGGAAGTTCCGGA |
| 127 | NLS linker | GSSGGSSG |
| 128 | truncated | GAGGGCCTATTTCCCATGATTCCTTCATATTTGCATATACGATACAAGGCTGTTAGAGAGATAATTGG |
| | hU6(249 bp) promoter | AATTAATTTGACTGTAAACACAAAGATATTAGTACAAAATACGTGACGTAGAAAGTAATAATTTCTT |
| | | GGGTAGTTTGCAGTTTTAAAATTATGTTTTAAAATGGACTATCATATGCTTACCGTAACTTGAAAGTA |
| | | TTTCGATTTCTTGGCTTTATATATCTTGTGGAAAGGACGAAACACC |
| 129 | SGN009849 | GAGUUUGUGACCCACGCCUGCUCCCGUUAUUGUACUCAAAGGAAUCUACAAUAAUAAGGCAUCUU |
| | | GCCGAAUUUACCGCCCUACAUAUGUAGGGCGGUUUU |
| 130 | SGN009849 target | GAGTTTGTGACCCACGCCTGCTCCC |
| | sequence | |
| 131 | SGN07707 | AGGAGGCAGCAGAGAACUCCCAGAAGUUAUUGUACUCAAAGGAAUCUACAAUAAUAAGGCAUCU |
| | | UGCCGAAUUUACCGCCCUACAUAUGUAGGGCGGUUUU |
| 132 | SGN07707 spacer | AGGAGGCAGCAGAGAACTCCCAGAA |
| | sequence | |
| 133 | APG07433.1 PAM- | GDLVRVDLFEKDDKYYMVPIYVPDTVCSELPKKVVASSKGYEQWLTLDNSFTFKFSLYPYDLVRLVKGD |
| | interacting domain | EDRFLYFGTLDIDSDRLNFKDVNKPSKKNEYRYSLKTIEDLEKYEVGVLGDLRLVRKETRRNFH |
| 134 | APG05586 PAM- | PYRYDIYQDLETKAYYTVQLYYSDLRFVEGEYGITEKEYMKKVAEQTKGQVVRFCFSLQKNDGLEIEWK |
| | interacting domain | DSQRYDVRFYNFQSANSINFKGLEQEMIPAENQFKQKPYNNGAINLNIAKYGKEGKKLRKFNTDILGKKH |
| | | HLSYEKEPKNIIK |
| 135 | APG01604 PAM- | PYRFDVYLTDKGYKFITISYLDVLKKDNYYYILKEKYEELKIKKSISDTDQFIGSFYYNDLIKINDQIFKVV |
| | interacting domain | GVNNDLLNRIELDLLDISYKEYCKINNIKTNRIIKGITKKITNIEKFSTDVLGNLYKAHSNHPQLIFKQRD |
| 136 | LPG10145 PAM- | PWRTDVYYNHEKEQYEIMGIKYADLKFKGDNYGITKARYQEIKEEEGVSEESEFLFSLYRGDRIQVSNGE |
| | interacting domain | DKIDLLFLSRSNPAKKGYVELKPIDRNQLNGKEVVSVYGAASGGRLKKQFVKKNHTLHKVNTDILGNPF |
| | | YIKKESDQPKNILDL |
| 137 | Nextera adapter left | TCGTCGGCAGCGTCAGATGTGTATAAGAGACAG |
| | primer sequence | |
| 138 | Nextera adapter right | GTCTCGTGGGCTCGGAGATGTGTATAAGAGACAG |
| | primer sequence | |
| 139 | conserved amino acid | LAGLIDADG |
| | motif | |
| 140 | APG05586 90 nt | GUUAUUGUACUCUCAAUAAAAAGUUAUUGAGAAUCUACAAUAAUAAGGCAUCUUGCCGAAUUUA |
| | backbone | CCGCCCUACAUAUGUAGGGCGGUUUU |
| 141 | APG05586 76 nt | GUUAUUGUACUCAAAGGAAUCUACAAUAAUAAGGCAUCUUGCCGAAUUUACCGCCCUACAUAUG |
| | backbone | UAGGGCGGUUUU |
| 142 | APG07433.1 110 nt | GUCAUAGUUCCAUUAAAGCCAAAAGUGGCUUUGAUGUUUCUAUGAUAAGGGUUUCGACCCGUGG |
| | backbone | CGUCGGGGAUCGCCUGCCCAUUGAAAUGGGCUUCUCCCCAUUUAUU |
| 143 | APG07433.1 RuvC-I | MRELDYRIGLDIGTNSIGWGVIELSWNKDRERYEKVRIVDQGVRMFDRAEMPKT |
| | domain | |
| 144 | APG07433.1 RuvC- | DTSGLKKEKRSKFLPPISDEITNPIVKRALTQARKVVNAIIRRHGSPHSVHIELARELS |
| | II domain | |
| 145 | APG07433.1 HNH | ILNPTGYDIVRYKLWKEQGERCAYSLKEIPADTFFNELKKERNGAPILEVDHILPYSQSFIDSYHNKVLVYS |
| | domain | DENRKKGNRIPYTYFLETNKDWEAFERYVRSNKFFSKKKREYLLKRAY |
| 146 | APG07433.1 RuvC- | NDTRYASTFLKNFIEQNLQFKEAEDNPRKRRVQTVNGVITAHFRKRWGLEKDRQETYLHHAMDAIIVAC |
| | III domain | TDHHMVTRVTEYYQIKESNKSVKKPYFPMPWEGFRDELLSHLASQPIAKKISEELKAGYQSLDYIFVSRM |
| | | PKRSITGAA |
| 147 | APG05586 RuvC-I | MYSIGLDLGISSVGWSVIDEETGKIVDLGVRLF |
| | domain | |
| 148 | APG05586 RuvC-II | KLPTKEVLAEIYNPTVNKTVSQAFKVMDALLEKYGKDQIHYITVEMPRD |
| | domain | |
| 149 | APG05586 HNH | QEKFQATIHKNRRFLAKLLYYFEQDGVCAYTGNPISPELLVSDSTEIDHIIPISISLDDSINNKVLVLSHANQ |
| | domain | VKGQQTPYDARMAGAFNKINGKFSNWDEYQKWVESRPFSRKKVNNLL |
| 150 | APG05586 RuvC-III | SRNLNDTRYASRLVLNTLQSFFENQDTIVRVVNGSFTHTLRKKWGADLDKTRETHHHHAVDATLCAVTP |
| | domain | FVKVSRYHYAVNEETGEKFMREIDVETGEILDEIPYREYKKAKHYERKTYQVKWSNFREQLKPITIHPKIK |
| | | FSHQVDRKAN |
| 151 | APG01604 RuvC-I | MVTKYILGLDIGITSVGYGIINYEDKTIIDAGVRLFPEAN |
| | domain | |
| 152 | APG01604 RuvC-II | DLAKTNKIPKNMIDEFILSPVVKRTFGQAINVINKVIEKYGVPEDIIIELARE |
| | domain | |
| 153 | APG01604 HNH | QNAKRLVEKIRLHDEQEGKCLYSLESIPLEDLINNPQYYEVDHIIPRSVSFDNSYQNKVLVKQTENSKKSN |
| | domain | RTPYQYFNSGETKLSYNQFKQHVLNLSKSKDRISKKKKEYLLEE |
| 154 | APG01604 RuvC-III | RYATRELTNYLKAYFSANDMDVKVKTINGSFTDYLRKVWKFKKERNHGYKHHAEDALIIANADFLFKE |
| | domain | NKKLKKANAILEQPSLDNGKSDATVENDNEYVETFSIPKQVNDIKEFRDFKFSHRV |
| 155 | LPG10145 RuvC-I | MSKVAKNMTRVNLGFDIGIASVGWSVLDNQTGKILETGVSIF |
| | domain | |
| 156 | LPG10145 RuvC-II | KVDVKYLTENLYNPVVRKSVKQAMDIFNALFEKYANIDYVVIEMPRDD |
| | domain | |
| 157 | LPG10145 HNH | DLQLEAQLRKRKKLRQKIRMWYQQRGKCPYSGKTIAAVDLFHQDNQFEIDHIIPLSVSFDDGQNNKVLC |
| | domain | YSEMNQEKGKQTPYAFMSRGGGQGFSALQAYVKSNNRLENAKKRNLL |
| 158 | LPG10145 RuvC-III | IARNLVDTRYASRIVLNELQQFVRSKELDTRVTVIRGKLTSKLRDRWRLNKSRETHHHHAVDAAVIAVSP |
| | domain | MLKMWEKNAEIIPLKVDENTVDLKSGEIITDQEYAAQMYELPYARFLEQMPELHKKIKFHHQVDKKMN |

That which is claimed:

1. An RNA-guided nuclease (RGN) system comprising:
a) a guide RNA comprising a spacer and a backbone, wherein said spacer has the nucleotide sequence of SEQ ID NO: 80 or 81 or a nucleotide sequence that differs from SEQ ID NO: 80 or 81 by 1 or 2 nucleotides, or a nucleic acid molecule encoding the guide RNA; and
b) an RGN polypeptide having an amino acid sequence having at least 90% sequence identity to SEQ ID NO: 7 or a nucleic acid molecule encoding the RGN polypeptide.

2. The RGN system of claim 1, wherein said guide RNA comprises a spacer having a nucleotide sequence that differs from SEQ ID NO: 80 or 81 by 1 nucleotide.

3. The RGN system of claim 1, wherein said guide RNA comprises a spacer having the nucleotide sequence of SEQ ID NO: 80 or 81.

4. The RGN system of any one of claims 1-3, wherein said guide RNA binds to a target sequence in a mutant huntingtin (mutHTT) allele.

5. The RGN system of claim 4, wherein said RGN system is capable of binding and cleaving a target sequence in said mutHTT allele, and wherein the guide RNA is capable of forming a complex with the RGN polypeptide and directing the complex to the target sequence for binding and cleaving.

6. The RGN system of claim 4 or 5, wherein said target sequence has the nucleotide sequence of SEQ ID NO: 75 or 76.

7. The RGN system of any one of claims 1-6, wherein said RGN system is capable of recognizing a protospacer adjacent motif (PAM) having the sequence of NNRYA created by a single nucleotide polymorphism (SNP) in exon 50 of the HTT gene.

8. The RGN system of any one of claims 1-7, wherein said RGN polypeptide comprises a PAM-interacting domain that binds a protospacer adjacent motif (PAM) having the sequence of NNRYA created by a single nucleotide polymorphism (SNP) in exon 50 of the HTT gene.

9. The RGN system of claim 8, wherein said PAM-interacting domain comprises an amino acid sequence having at least 95% sequence identity to SEQ ID NO: 134.

10. The RGN system of claim 8 or 9, wherein said PAM-interacting domain comprises the amino acid sequence set forth as SEQ ID NO: 134.

11. The RGN system of any one of claims 1-10, wherein said RGN polypeptide comprises at least one nuclease domain comprising an amino acid sequence having at least 95% sequence identity to any one of SEQ ID NOs: 147, 148, 149, and 150.

12. The RGN system of any one of claims 1-11, wherein said RGN system is not capable of cleaving a wild type HTT allele.

13. The RGN system of any one of claims 1-12, wherein said RGN polypeptide has an amino acid sequence having at least 95% sequence identity to SEQ ID NO: 7.

14. The RGN system of any one of claims 1-13, wherein said RGN polypeptide has the amino acid sequence of SEQ ID NO: 7.

15. The RGN system of any one of claims 1-14, wherein said RGN polypeptide further comprises at least one nuclear localization signal.

16. The RGN system of claim 15, wherein said at least one nuclear localization signal comprises an SV40 nuclear localization signal.

17. The RGN system of claim 16, wherein said SV40 nuclear localization signal has the sequence set forth as SEQ ID NO: 86.

18. The RGN system of claim 15, wherein said at least one nuclear localization signal comprises a c-Myc nuclear localization signal.

19. The RGN system of claim 18, wherein said c-Myc nuclear localization signal has the sequence set forth as SEQ ID NO: 125.

20. The RGN system of any one of claims 15-19, wherein a NLS linker protein connects said RGN polypeptide and said at least one nuclear localization signal.

21. The RGN system of claim 20, wherein said NLS linker protein has the sequence set forth as SEQ ID NO: 127.

22. The RGN system of any one of claims 1-21, wherein the backbone of the guide RNA is 66 to 90 nucleotides in length.

23. The RGN system of any one of claims 1-22, wherein the backbone of the guide RNA comprises a nucleotide sequence having at least 80% sequence identity to SEQ ID NO: 140 or 141.

24. The RGN system of any one of claims 1-23, wherein said guide RNA comprises a crRNA repeat having the nucleotide sequence of SEQ ID NO: 8 or 106 or a nucleotide sequence that differs from SEQ ID NO: 8 or 106 by 1 or 2 nucleotides and a tracrRNA having a nucleotide sequence having at least 90% sequence identity to SEQ ID NO: 9 or 107.

25. The RGN system of any one of claims 1-23, wherein said guide RNA comprises a crRNA repeat having a nucleotide sequence that differs from SEQ ID NO: 8 or 106 by 1 nucleotide and a tracrRNA having a nucleotide sequence having at least 95% sequence identity to SEQ ID NO: 9 or 107.

26. The RGN system of any one of claims 1-23, wherein said guide RNA comprises a crRNA repeat having the nucleotide sequence of SEQ ID NO: 8 or 106 and a tracrRNA having the nucleotide sequence of SEQ ID NO: 9 or 107.

27. The RGN system of any one of claims 1-26, wherein said guide RNA is a single guide RNA.

28. The RGN system of claim 27, wherein said single guide RNA has the nucleotide sequence of SEQ ID NO: 25 or 26.

29. A ribonucleoprotein (RNP) complex comprising the RGN polypeptide and the guide RNA of said RGN system of any one of claims 1-28.

30. A nucleic acid molecule comprising or encoding a guide RNA that comprises a spacer having the nucleotide sequence of SEQ ID NO: 80 or 81 or a nucleotide sequence that differs from SEQ ID NO: 80 or 81 by 1 or 2 nucleotides.

31. The nucleic acid molecule of claim 30, wherein said guide RNA comprises a spacer having a nucleotide sequence that differs from SEQ ID NO: 80 or 81 by 1 nucleotide.

32. The nucleic acid molecule of claim 30, wherein said guide RNA comprises a spacer having the nucleotide sequence of SEQ ID NO: 80 or 81.

33. The nucleic acid molecule of any one of claims 30-32, wherein said guide RNA binds to a target sequence in a mutant huntingtin (mutHTT) allele.

34. The nucleic acid molecule of claim 33, wherein said target sequence has the nucleotide sequence of SEQ ID NO: 75 or 76.

35. The nucleic acid molecule of any one of claims 30-34, wherein said guide RNA binds to an RNA-guided nuclease (RGN) polypeptide having an amino acid sequence having at least 90% sequence identity to SEQ ID NO: 7.

36. A nucleic acid molecule comprising or encoding a guide RNA that binds to a target sequence in a mutant huntingtin (mutHTT) allele, wherein said target sequence has the nucleotide sequence of SEQ ID NO: 75 or 76 and binds to an RNA-guided nuclease (RGN) polypeptide having an amino acid sequence having at least 90% sequence identity to SEQ ID NO: 7.

37. The nucleic acid molecule of claim 36, wherein said guide RNA comprises a spacer having the nucleotide sequence of SEQ ID NO: 80 or 81 or a nucleotide sequence that differs from SEQ ID NO: 80 or 81 by 1 or 2 nucleotides.

38. The nucleic acid molecule of claim 37, wherein said guide RNA comprises a spacer having a nucleotide sequence that differs from SEQ ID NO: 80 or 81 by 1 nucleotide.

39. The nucleic acid molecule of claim 37, wherein said guide RNA comprises a spacer having the nucleotide sequence of SEQ ID NO: 80 or 81.

40. The nucleic acid molecule of any one of claims 35-39, wherein said RGN polypeptide has an amino acid sequence having at least 95% sequence identity to SEQ ID NO: 7.

41. The nucleic acid molecule of any one of claims 35-40, wherein said RGN polypeptide has the amino acid sequence of SEQ ID NO: 7.

42. The nucleic acid molecule of any one of claims 30-41, wherein said guide RNA comprises a crRNA repeat having the nucleotide sequence of SEQ ID NO: 8 or 106 or a nucleotide sequence that differs from SEQ ID NO: 8 or 106 by 1 or 2 nucleotides and a tracrRNA having a nucleotide sequence having at least 90% sequence identity to SEQ ID NO: 9 or 107.

43. The nucleic acid molecule of any one of claims 30-41, wherein said guide RNA comprises a crRNA repeat having a nucleotide sequence that differs from SEQ ID NO: 8 or 106 by 1 nucleotide and a tracrRNA having a nucleotide sequence having at least 95% sequence identity to SEQ ID NO: 9 or 107.

44. The nucleic acid molecule of any one of claims 30-41, wherein said guide RNA comprises a crRNA repeat having the nucleotide sequence of SEQ ID NO: 8 or 106 and a tracrRNA having the nucleotide sequence of SEQ ID NO: 9 or 107.

45. The nucleic acid molecule of any one of claims 30-44, wherein said guide RNA is a single guide RNA.

46. The nucleic acid molecule of claim 45, wherein said single guide RNA has the nucleotide sequence of SEQ ID NO: 25 or 26.

47. The nucleic acid molecule of any one of claims 30-46, wherein said nucleic acid molecule encoding said guide RNA is operably linked to an RNA polymerase III promoter.

48. The nucleic acid molecule of claim 47, wherein said RNA polymerase III promoter is a U6 promoter.

49. The nucleic acid molecule of claim 48, wherein said U6 promoter is a truncated U6 promoter.

50. The nucleic acid molecule of claim 49, wherein said truncated U6 promoter has the nucleotide sequence set forth as SEQ ID NO: 89 or 128.

51. A vector comprising the nucleic acid molecule of any one of claims 30-35, wherein the nucleic acid molecule encodes the guide RNA.

52. A vector comprising the nucleic acid molecule of any one of claims 36-50, wherein the nucleic acid molecule encodes the guide RNA.

53. The vector of claim 51 or 52, wherein said vector is a viral vector.

54. The vector of claim 53, wherein said viral vector is a lentiviral vector, a baculoviral vector, or an adeno-associated viral (AAV) vector.

55. The vector of claim 54, wherein said viral vector is an AAV vector and comprises AAV inverted terminal repeats.

56. The vector of claim 55, wherein said AAV inverted terminal repeats are AAV2, AAV5 or AAV6 inverted terminal repeats.

57. The vector of any one of claims 52-56, wherein the vector further comprises a nucleic acid molecule encoding said RGN polypeptide.

58. The vector of claim 57, wherein the vector further comprises an RNA polymerase II promoter operably linked to the nucleic acid molecule encoding the RGN polypeptide.

59. The vector of claim 58, wherein said RNA polymerase II promoter is a constitutive promoter.

60. The vector of claim 59, wherein said constitutive promoter is selected from the group consisting of: a cytomegalovirus (CMV) promoter, a truncated CMV promoter, an elongation factor 1α short (EFS) promoter, and a JeT promoter.

61. The vector of claim 60, wherein said constitutive promoter is a JeT promoter.

62. The vector of claim 61, wherein said JeT promoter has the nucleotide sequence set forth as SEQ ID NO: 92.

63. The vector of claim 58, wherein said RNA polymerase II promoter is a tissue-specific promoter.

64. The vector of claim 63, wherein said tissue-specific promoter is a brain or neuron specific promoter.

65. The vector of claim 64, wherein said brain or neuron specific promoter is selected from the group consisting of: a human synapsin I (Syn) promoter, a 67 kDa glutamic acid decarboxylase (GAD67) promoter, a 65 kDa glutamic acid decarboxylase (GAD65) promoter, a homeobox Dlx5/6 promoter, a preprotachykinin 1 (Tacd) promoter, a neuron-specific enolase (NSE) promoter, a dopaminergic receptor 1 (Drd1a) promoter, a dopaminergic receptor 2 (DRD2) promoter, and a glial fibrillary acidic protein (GFAP) promoter.

66. The vector of claim 65, wherein said neuron specific promoter is a Syn promoter.

67. The vector of claim 66, wherein said Syn promoter has the nucleotide sequence set forth as SEQ ID NO: 93.

68. The vector of any one of claims 57-67, wherein said nucleic acid molecule encoding said RGN polypeptide comprises a polyadenylation (polyA) tail.

69. The vector of claim 68, wherein said polyA tail is a SV40 polyA tail or a bovine growth hormone (bGH) polyA tail.

70. The vector of claim 69, wherein said SV40 polyA tail has the nucleotide sequence set forth as SEQ ID NO: 94.

71. The vector of claim 69, wherein said bGH polyA tail has the sequence set forth as SEQ ID NO: 95.

72. The vector of claim 57 or 58, wherein said vector comprises: a truncated U6 promoter operably linked to said nucleic acid molecule encoding said guide RNA; a CMVeb promoter operably linked to said nucleic acid molecule encoding said RGN polypeptide; a c-Myc NLS at the N-terminus and C-terminus of said RGN polypeptide; an NLS linker protein connecting said c-Myc NLS and said RGN polypeptide; and an SV40 polyA tail.

73. The vector of claim 72, wherein said truncated U6 promoter has the sequence set forth as SEQ ID NO: 128, said CMVeb promoter has the sequence set forth as SEQ ID NO: 90, said c-Myc NLS has the sequence set forth as SEQ ID NO: 125, said NLS linker protein has the sequence set forth as SEQ ID NO: 127, and said SV40 polyA tail has the sequence set forth as SEQ ID NO: 94.

74. The vector of claim 72 or 73, wherein said sgRNA has the sequence set forth as SEQ ID NO: 26 and said nucleic acid molecule encoding said RGN polypeptide has the sequence set forth as SEQ ID NO: 88.

75. The vector of any one of claims 72-74, wherein said vector comprises the sequence set forth as SEQ ID NO: 123.

76. The vector of any one of claims 57-75, wherein said RGN polypeptide is operably linked to at least one nuclear localization signal.

77. The vector of claim 76, wherein said at least one nuclear localization signal comprises an SV40 nuclear localization signal.

78. The vector of claim 77, wherein said SV40 nuclear localization signal has the sequence set forth as SEQ ID NO: 86.

79. The vector of claim 76, wherein said at least one nuclear localization signal comprises a c-Myc nuclear localization signal.

80. The vector of claim 79, wherein said c-Myc nuclear localization signal has the sequence set forth as SEQ ID NO: 125.

81. The vector of any one of claims 76-80, wherein a NLS linker protein connects said RGN polypeptide and said at least one nuclear localization signal.

82. The vector of claim 81, wherein said NLS linker protein has the sequence set forth as SEQ ID NO: 127.

83. The vector of any one of claims 57-82, wherein said vector has the sequence set forth as any one of SEQ ID NOs: 32-39 or 121-123.

84. An RNA-guided nuclease (RGN) system comprising:
a) a guide RNA comprising having the nucleotide sequence of SEQ ID NO: 82 or 83 or a nucleotide sequence that differs from SEQ ID NO: 82 or 83 by 1 or 2 nucleotides, or a nucleic acid molecule encoding the guide RNA; and
b) an RGN polypeptide having an amino acid sequence having at least 90% sequence identity to SEQ ID NO: 3 or a nucleic acid molecule encoding the RGN polypeptide.

85. The RGN system of claim 84, wherein said guide RNA comprises a spacer having a nucleotide sequence that differs from SEQ ID NO: 82 or 83 by 1 nucleotide.

86. The RGN system of claim 84, wherein said guide RNA comprises a spacer having the nucleotide sequence of SEQ ID NO: 82 or 83.

87. The RGN system of any one of claims 84-86, wherein said guide RNA binds to a target sequence in a mutant huntingtin (mutHTT) allele.

88. The RGN system of claim 87, wherein said system is capable of binding and cleaving a target sequence in said mutHTT allele, and wherein the guide RNA is capable of forming a complex with the RGN polypeptide and directing the complex to the target sequence for binding and cleaving.

89. The RGN system of claim 87 or 88, wherein said target sequence has the nucleotide sequence of SEQ ID NO: 77 or 78.

90. The RGN system of any one of claims 84-89, wherein said RGN system is capable of recognizing a protospacer adjacent motif (PAM) having the sequence of NNNNCC created by a single nucleotide polymorphism (SNP) in exon 50 of the HTT gene.

91. The RGN system of any one of claims 84-90, wherein said RGN comprises a PAM-interacting domain that binds a protospacer adjacent motif (PAM) having the sequence of NNNNCC created by a single nucleotide polymorphism (SNP) in exon 50 of the HTT gene.

92. The RGN system of claim 91, wherein said PAM-interacting domain comprises an amino acid sequence having at least 95% sequence identity to SEQ ID NO: 133.

93. The RGN system of claim 91 or 92, wherein said PAM-interacting domain comprises the amino acid sequence set forth as SEQ ID NO: 133.

94. The RGN system of any one of claims 84-93, wherein said RGN polypeptide comprises at least one nuclease domain comprising an amino acid sequence having at least 95% sequence identity to any one of SEQ ID NOs: 143, 144, 145, and 146.

95. The RGN system of any one of claims 84-94, wherein said RGN system is not capable of cleaving a wild type HTT allele.

96. The RGN system of any one of claims 84-95, wherein said RGN polypeptide has an amino acid sequence having at least 95% sequence identity to SEQ ID NO: 3.

97. The RGN system of any one of claims 84-96, wherein said RGN polypeptide has the amino acid sequence of SEQ ID NO: 3.

98. The RGN system of any one of claims 84-97, wherein said RGN polypeptide comprises at least one nuclear localization signal.

99. The RGN system of claim 98, wherein said at least one nuclear localization signal comprises an SV40 nuclear localization signal.

100. The RGN system of claim 99, wherein said SV40 nuclear localization signal has the sequence set forth as SEQ ID NO: 86.

101. The RGN system of claim 98, wherein said at least one nuclear localization signal comprises a c-Myc nuclear localization signal.

102. The RGN system of claim 101, wherein said c-Myc nuclear localization signal has the sequence set forth as SEQ ID NO: 125.

103. The RGN system of any one of claims 98-102, wherein a NLS linker protein connects said RGN polypeptide and said at least one nuclear localization signal.

104. The RGN system of claim 103, wherein said NLS linker protein has the sequence set forth as SEQ ID NO: 127.

105. The RGN system of any one of claims 84-104, wherein the backbone of the guide RNA is 94 to 110 nucleotides in length.

106. The RGN system of any one of claims 84-105, wherein the backbone of the guide RNA comprises a nucleotide sequence having at least 80% sequence identity to SEQ ID NO: 142.

107. A ribonucleoprotein (RNP) complex comprising the RGN polypeptide and the guide RNA of said RGN system of any one of claims 84-106.

108. A nucleic acid molecule comprising or encoding a guide RNA that comprises a spacer having the nucleotide sequence of SEQ ID NO: 82 or 83 or a nucleotide sequence that differs from SEQ ID NO: 82 or 83 by 1 or 2 nucleotides.

109. The nucleic acid molecule of claim 108, wherein said guide RNA comprises a spacer having a nucleotide sequence that differs from SEQ ID NO: 82 or 83 by 1 nucleotide.

110. The nucleic acid molecule of claim 108, wherein said guide RNA comprises a spacer having the nucleotide sequence of SEQ ID NO: 82 or 83.

111. The nucleic acid molecule of any one of claims 108-110, wherein said guide RNA binds to a target sequence in a mutant huntingtin (mutHTT) allele.

112. The nucleic acid molecule of claim 111, wherein said target sequence has the nucleotide sequence of SEQ ID NO: 77 or 78.

113. The nucleic acid molecule of any one of claims 108-112, wherein said guide RNA binds to an RNA-guided nuclease (RGN) polypeptide having an amino acid sequence having at least 90% sequence identity to SEQ ID NO: 3.

114. A nucleic acid molecule comprising or encoding a guide RNA that binds to a target sequence in a mutant huntingtin (mutHTT) allele, wherein said target sequence has the nucleotide sequence of SEQ ID NO: 77 or 78 and binds to an RNA-guided nuclease (RGN) polypeptide having an amino acid sequence having at least 90% sequence identity to SEQ ID NO: 3.

115. The nucleic acid molecule of claim 114, wherein said guide RNA comprises a spacer having the nucleotide sequence of SEQ ID NO: 82 or 83 or a nucleotide sequence that differs from SEQ ID NO: 82 or 83 by 1 or 2 nucleotides.

116. The nucleic acid molecule of claim 115, wherein said guide RNA comprises a spacer having a nucleotide sequence that differs from SEQ ID NO: 82 or 83 by 1 nucleotide.

117. The nucleic acid molecule of claim 115, wherein said guide RNA comprises a spacer having the nucleotide sequence of SEQ ID NO: 82 or 83.

118. The nucleic acid molecule of any one of claims 113-117, wherein said RGN polypeptide has an amino acid sequence having at least 95% sequence identity to SEQ ID NO: 3.

119. The nucleic acid molecule of any one of claims 113-118, wherein said RGN polypeptide has the amino acid sequence of SEQ ID NO: 3.

120. The nucleic acid molecule of any one of claims 108-119, wherein said guide RNA comprises a crRNA repeat having the nucleotide sequence of SEQ ID NO: 4 or a nucleotide sequence that differs from SEQ ID NO: 4 by 1 or 2 nucleotides and a tracrRNA having a nucleotide sequence having at least 90% sequence identity to SEQ ID NO: 5.

121. The nucleic acid molecule of any one of claims 108-119, wherein said guide RNA comprises a crRNA repeat having a nucleotide sequence that differs from SEQ ID NO: 4 by 1 nucleotide and a tracrRNA having a nucleotide sequence having at least 95% sequence identity to SEQ ID NO: 5.

122. The nucleic acid molecule of any one of claims 108-119, wherein said guide RNA comprises a crRNA repeat having the nucleotide sequence of SEQ ID NO: 4 and a tracrRNA having the nucleotide sequence of SEQ ID NO: 5.

123. The nucleic acid molecule of any one of claims 108-122, wherein said guide RNA is a single guide RNA.

124. The nucleic acid molecule of claim 123, wherein said single guide RNA has the nucleotide sequence of SEQ ID NO: 27 or 28.

125. The nucleic acid molecule of any one of claims 108-124, wherein said nucleic acid molecule encoding said guide RNA is operably linked to an RNA polymerase III promoter.

126. The nucleic acid molecule of claim 125, wherein said RNA polymerase III promoter is a U6 promoter.

127. The nucleic acid molecule of claim 126, wherein said U6 promoter is a truncated U6 promoter.

128. The nucleic acid molecule of claim 127, wherein said truncated U6 promoter has the nucleotide sequence set forth as SEQ ID NO: 89 or 128.

129. A vector comprising the nucleic acid molecule of any one of claims 108-112, wherein the nucleic acid molecule encodes the guide RNA.

130. A vector comprising the nucleic acid molecule of any one of claims 113-128, wherein the nucleic acid molecule encodes the guide RNA.

131. The vector of claim 129 or 130, wherein said vector is a viral vector.

132. The vector of claim 131, wherein said viral vector is a lentiviral vector, a baculoviral vector, or an adeno-associated viral (AAV) vector.

133. The vector of claim 132, wherein said viral vector is an AAV vector and comprises AAV inverted terminal repeats.

134. The vector of claim 133, wherein said AAV inverted terminal repeats are AAV2, AAV5 or AAV6 inverted terminal repeats.

135. The vector of any one of claims 130-134, wherein the vector further comprises a nucleic acid molecule encoding said RGN polypeptide.

136. The vector of claim 135, wherein the vector further comprises an RNA polymerase II promoter operably linked to the nucleic acid molecule encoding the RGN polypeptide.

137. The vector of claim 136, wherein said RNA polymerase II promoter is a constitutive promoter.

138. The vector of claim 137, wherein said constitutive promoter is selected from the group consisting of: a cytomegalovirus (CMV) promoter, a truncated CMV promoter, an elongation factor 1α short (EFS) promoter, and a JeT promoter.

139. The vector of claim 138, wherein said constitutive promoter is a JeT promoter.

140. The vector of claim 139, wherein said JeT promoter has the nucleotide sequence set forth as SEQ ID NO: 92.

141. The vector of claim 136, wherein said RNA polymerase II promoter is a tissue-specific promoter.

142. The vector of claim 141, wherein said tissue-specific promoter is a brain or neuron specific promoter.

143. The vector of claim 142, wherein said brain or neuron specific promoter is selected from the group consisting of: a human synapsin I (Syn) promoter, a 67 kDa glutamic acid decarboxylase (GAD67) promoter, a 65 kDa glutamic acid decarboxylase (GAD65) promoter, a homeobox Dlx5/6 promoter, a preprotachykinin 1 (Tacd) promoter, a neuron-specific enolase (NSE) promoter, a dopaminergic receptor 1 (Drd1a) promoter, a dopaminergic receptor 2 (DRD2) promoter, and a glial fibrillary acidic protein (GFAP) promoter.

144. The vector of claim 143, wherein said neuron specific promoter is a Syn promoter.

145. The vector of claim 144, wherein said Syn promoter has the nucleotide sequence set forth as SEQ ID NO: 93.

146. The vector of any one of claims 135-145, wherein said nucleic acid molecule encoding said RGN polypeptide comprises a polyadenylation (polyA) tail.

147. The vector of claim 146, wherein said polyA tail is a SV40 polyA tail or a bovine growth hormone (bGH) polyA tail.

148. The vector of claim 147, wherein said SV40 polyA tail has the nucleotide sequence set forth as SEQ ID NO: 94.

149. The vector of claim 147, wherein said bGH polyA tail has the sequence set forth as SEQ ID NO: 95.

150. The vector of any one of claims 135-149, wherein said RGN polypeptide is operably linked to at least one nuclear localization signal.

151. The vector of claim 150, wherein said at least one nuclear localization signal comprises an SV40 nuclear localization signal.

152. The vector of claim 151, wherein said SV40 nuclear localization signal has the sequence set forth as SEQ ID NO: 86.

153. The vector of claim 150, wherein said at least one nuclear localization signal comprises a c-Myc nuclear localization signal.

154. The vector of claim 153, wherein said c-Myc nuclear localization signal has the sequence set forth as SEQ ID NO: 125.

155. The vector of any one of claims 150-154, wherein a NLS linker protein connects said RGN polypeptide and said at least one nuclear localization signal.

156. The vector of claim 155, wherein said NLS linker protein has the sequence set forth as SEQ ID NO: 127.

157. A cell comprising the nucleic acid molecule of any one of claims 30-50 and 108-128 or the vector of any one of claims 51-83 and 129-156.

158. A pharmaceutical composition comprising the nucleic acid molecule of any one of claims 30-50 and 108-128, the vector of any one of claims 51-83 and 129-156, the RGN system of any one of claims 1-28 and 84-106, or the RNP complex of claim 29 or 107.

159. The pharmaceutical composition of claim 158 having a purity of at least 95%.

160. The pharmaceutical composition of claim 158 or 159 having undetectable levels of endotoxin or other impurities.

161. The pharmaceutical composition of any one of claims 158-160, further comprising poloxamer 188.

162. The pharmaceutical composition of any of claims 158-161 that is in solution.

163. The pharmaceutical composition of any of claims 158-161 that is lyophilized or freeze-dried.

164. A vector comprising the RGN system of any one of claims 1-28.

165. A vector comprising the RGN system of any one of claims 84-106.

166. The vector of claim 164 or 165, wherein said vector comprises adeno-associated vector (AAV) inverted terminal repeats.

167. The vector of claim 166, wherein said AAV inverted terminal repeats are AAV2, AAV5, or AAV6 inverted terminal repeats.

168. The vector of claim 167, wherein said AAV inverted terminal repeats are AAV5 inverted terminal repeats.

169. Use of the nucleic acid molecule of any one of claims 30-50 and 108-128, the vector of any one of claims 51-83, 129-156, and 164-168, the RGN system of any one of claims 1-28 and 84-106, or the RNP complex of claim 29 or 107 for reducing a level of mutHTT mRNA and/or mutHTT protein in a cell.

170. Use of the nucleic acid molecule of any one of claims 30-50 and 108-128, the vector of any one of claims 51-83, 129-156, and 164-168, the RGN system of any one of claims 1-28 and 84-106, or the RNP complex of claim 29 or 107 for treating Huntington's disease.

171. A method for cleaving a mutant huntingtin (mutHTT) allele in a cell, wherein said mutHTT allele comprises a first single nucleotide polymorphism (SNP) allele in exon 50, wherein a protospacer adjacent motif (PAM) having the nucleotide sequence of NNRYA comprises said first SNP allele, wherein said method comprises introducing into the cell:
a) an RNA-guided nuclease (RGN) polypeptide having at least 90% sequence identity to SEQ ID NO: 7 or a nucleic acid molecule encoding said RGN polypeptide, and
i) the nucleic acid molecule comprising or encoding a guide RNA of any one of claims 30-50; or
ii) the vector of any one of claims 51-56;
b) the vector of any one of claims 57-83, 164, and 166-168;
c) the RGN system of any one of claims 1-28; or
d) the RNP complex of claim 29.

172. A method for cleaving a mutant huntingtin (mutHTT) allele in a cell, wherein said mutHTT allele comprises a first single nucleotide polymorphism (SNP) allele in exon 50, wherein a protospacer adjacent motif (PAM) having the nucleotide sequence of NNNNCC comprises said first SNP allele, wherein said method comprises introducing into the cell:
a) an RNA-guided nuclease (RGN) polypeptide having at least 90% sequence identity to SEQ ID NO: 3 or a nucleic acid molecule encoding said RGN polypeptide, and
i) the nucleic acid molecule comprising or encoding a guide RNA of any one of claims 108-128; or
ii) the vector of any one of claims 129-134;
b) the vector of any one of claims 135-156, and 165-168;
c) the RGN system of any one of claims 84-106; or
d) the RNP complex of claim 107.

173. The method of claim 171 or 172, wherein said RGN polypeptide is capable of recognizing said PAM and cleaving said mutHTT allele.

174. The method of any one of claims 171-173, wherein said mutHTT allele has at least 36 CAG repeats in exon 1.

175. The method of claim any one of claims 171-173, wherein said mutHTT allele has at least 40 CAG repeats in exon 1.

176. The method of any one of claims 171-175, wherein said cell has been assayed to determine whether said mutHTT allele comprises said first SNP allele prior to the introduction of said RGN polypeptide or said nucleic acid molecule encoding said RGN polypeptide and said guide RNA or said nucleic acid molecule encoding said guide RNA, said vector, said RGN system, or said RNP complex.

177. The method of any one of claims 171-176, wherein said cell comprises a wild-type HTT (wtHTT) allele comprising a second SNP allele where said PAM is not present, and said cell is thereby heterozygous for the SNP.

178. The method of claim 177, wherein said cell has been assayed to determine whether said cell is heterozygous for the SNP prior to the introduction of said RGN polypeptide or said nucleic acid molecule encoding said RGN polypeptide and said guide RNA or said nucleic acid molecule encoding said guide RNA, said vector, said RGN system, or said RNP complex.

179. The method of any one of claims 171-178, wherein said mutHTT allele is edited, thereby creating a genetically modified cell comprising said edited mutHTT allele.

180. The method of claim 179, wherein said editing comprises introducing an insertion and/or deletion (INDEL) at or near said SNP.

181. The method of claim 179, wherein said editing comprises introducing a premature stop codon at or near said SNP.

182. The method of any one of claims 179-181, wherein said genetically modified cell is a genetically modified stem cell.

183. The method of claim 182, wherein said genetically modified stem cell is a genetically modified induced pluripotent stem cell (iPSC) or a genetically modified mesenchymal stem cell (MSC).

184. The method of claim 183, wherein said method further comprises differentiating the genetically modified iPSC or MSC into a neuronal cell.

185. The method of any one of claims 179-184, wherein a level of mutHTT mRNA is reduced by at least 40% as compared to a level of HTT mRNA in a non-genetically modified cell or to a level of wild type HTT mRNA.

186. The method of any one of claims 179-185, wherein a level of mutHTT protein is reduced by at least 40% as compared to a level of HTT protein in a non-genetically modified cell or to a level of wild type HTT protein.

187. The method of any one of claims 179-186, further comprising selecting said genetically modified cell.

188. A genetically modified cell produced by the method of claim 187.

189. The method of any one of claims 171-187, wherein said introducing comprises administering a composition comprising said RGN polypeptide or said nucleic acid molecule encoding said RGN polypeptide and said guide RNA or said nucleic acid molecule encoding said guide RNA, said vector, said RGN system, or said RNP complex, to a subject comprising said cell.

190. The method of claim 189, wherein the cell is a eukaryotic cell.

191. The method of claim 190, wherein the eukaryotic cell is a mammalian cell.

192. The method of claim 191, wherein the mammalian cell is a human cell.

193. The method of claim 191 or 192, wherein the mammalian cell or human cell is a stem cell.

194. The method of claim 191 or 192, wherein the mammalian cell or human cell is a forebrain neuron, a striatal neuron, a medium spiny neuron, a cortical neuron, or a glial cell.

195. The method of claim 191 or 192, wherein the mammalian cell or human cell is present in putamen, caudate, striatum, cerebral cortex, globus pallidus, hippocampus, amygdala, thalamus, hypothalamus, subthalamic nucleus, substantia nigra, cerebellum, brainstem, or a combination thereof.

196. A method for ameliorating or delaying the onset of one or more symptoms of Huntington's disease (HD) in a subject in need thereof, wherein said subject comprises a mutant huntingtin (mutHTT) allele comprising:
a) at least 36 CAG repeats in exon 1; and
b) a first single nucleotide polymorphism (SNP) allele in exon 50, wherein a protospacer adjacent motif (PAM) having the nucleotide sequence of NNRYA comprises said first SNP allele;
wherein said method comprises administering to said subject:
a) an RNA-guided nuclease (RGN) polypeptide having at least 90% sequence identity to SEQ ID NO: 7 or a nucleic acid molecule encoding said RGN polypeptide, and
i) the nucleic acid molecule comprising or encoding a guide RNA of any one of claims 30-50; or
ii) the vector of any one of claims 51-56;
b) the vector of any one of claims 57-83, 164, and 166-168;
c) the RGN system of any one of claims 1-28; or
d) the RNP complex of claim 29;
and wherein the level of a mutHTT protein encoded by said mutHTT allele is reduced as compared to a level of HTT protein of a control subject or a level of wild type HTT protein.

197. A method for ameliorating or delaying the onset of one or more symptoms of Huntington's disease (HD) in a subject in need thereof, wherein said subject comprises a mutant huntingtin (mutHTT) allele comprising:
a) at least 36 CAG repeats in exon 1; and
b) a first single nucleotide polymorphism (SNP) allele in exon 50, wherein a protospacer adjacent motif (PAM) having the nucleotide sequence of NNNNCC comprises said first SNP allele;
wherein said method comprises administering to said subject:
a) an RNA-guided nuclease (RGN) polypeptide having at least 90% sequence identity to SEQ ID NO: 3 or a nucleic acid molecule encoding said RGN polypeptide, and
i) the nucleic acid molecule comprising or encoding a guide RNA of any one of claims 108-128; or
ii) the vector of any one of claims 129-134;
b) the vector of any one of claims 135-156, and 165-168;
c) the RGN system of any one of claims 84-106; or
d) the RNP complex of claim 107;
and wherein the level of a mutHTT protein encoded by said mutHTT allele is reduced as compared to a level of HTT protein of a control subject or a level of wild type HTT protein.

198. The method of claim 196 or 197, wherein said RGN polypeptide recognizes said PAM and cleaves and edits said mutHTT allele.

199. The method of any one of claims 196-198, wherein said administering comprises intrastriatal, intraparenchymal, intrathecal, intracerebral, intracerebroventricular, intrathalamic, or intra-cisterna magna injection.

200. The method of any one of claims 196-199, wherein said subject has been assayed to determine whether said mutHTT allele comprises said first SNP allele comprising said PAM prior to the administration of said RGN polypeptide or said nucleic acid molecule encoding said RGN polypeptide and said guide RNA or said nucleic acid molecule encoding said guide RNA, said vector, said RGN system, or said RNP complex.

201. The method of any one of claims 196-200, wherein the subject comprises a wild-type HTT (wtHTT) allele comprising a second SNP allele where said PAM is not present, and said subject is thereby heterozygous for the SNP.

202. The method of claim 201, wherein said subject has been assayed to determine whether said subject is heterozygous for the SNP prior to the administration of said RGN polypeptide or said nucleic acid molecule encoding said RGN polypeptide and said guide RNA or said nucleic acid molecule encoding said guide RNA, said vector, said RGN system, or said RNP complex.

203. The method of any one of claims 196-202, wherein said PAM is present only on the mutHTT allele and not the wild-type HTT allele.

204. A method of ameliorating or delaying the onset of one or more symptoms of Huntington's disease (HD) in a subject in need thereof, wherein said subject comprises a mutant huntingtin (mutHTT) allele comprising:
a) at least 36 CAG repeats in exon 1; and
b) a first single nucleotide polymorphism (SNP) allele in exon 50, wherein said SNP allele comprises a thymine at a position corresponding to position 151 of SEQ ID NO: 1;
wherein said method comprises administering by intrastriatal injection into said subject an AAV5 vector comprising:
a) a first nucleic acid molecule encoding an RNA-guided nuclease (RGN) polypeptide having the amino acid sequence of SEQ ID NO: 7; and
b) a second nucleic acid molecule encoding a guide RNA having the nucleotide sequence of SEQ ID NO: 25 or 26; and
wherein 4-weeks post-administration of said AAV5 vector, said subject has a decrease in a level of mutHTT protein encoded by said mutHTT allele as compared to a level of HTT protein of a control subject or a level of wild type HTT protein.

205. A method of ameliorating or delaying the onset of one or more symptoms of Huntington's disease (HD) in a subject in need thereof, wherein said subject comprises a mutant huntingtin (mutHTT) allele comprising:
a) at least 36 CAG repeats in exon 1; and
b) a first single nucleotide polymorphism (SNP) allele in exon 50, wherein said first SNP allele comprises a cytosine at a position corresponding to position 151 of SEQ ID NO: 2;
wherein said method comprises administering by intrastriatal injection into said subject an AAV5 vector comprising:
a) a first nucleic acid molecule encoding an RNA-guided nuclease (RGN) polypeptide having the amino acid sequence of SEQ ID NO: 3; and
b) a second nucleic acid molecule encoding a guide RNA having the nucleotide sequence of SEQ ID NO: 27 or 28; and
wherein 4 weeks post-administration of said AAV5 vector, said subject has a decrease in a level of mutHTT protein encoded by said mutHTT allele as compared to a level of HTT protein of a control subject or a level of wild type HTT protein.

206. A method of ameliorating or delaying the onset of one or more symptoms of Huntington's disease (HD) in a subject in need thereof, said method comprises:
a) selecting a subject comprising a mutant huntingtin (mutHTT) allele comprising:
i) at least 36 CAG repeats in exon 1;
ii) a first single nucleotide polymorphism (SNP) allele in exon 50, wherein said SNP allele comprises a thymine at a position corresponding to position 151 of SEQ ID NO: 1; and
b) administering by intrastriatal injection into said subject an AAV5 vector comprising:
i) a first nucleic acid molecule encoding an RNA-guided nuclease (RGN) polypeptide having the amino acid sequence of SEQ ID NO: 7; and
ii) a second nucleic acid molecule encoding a guide RNA having the nucleotide sequence of SEQ ID NO: 25 or 26; and
wherein 4 weeks post-administration of said AAV5 vector, said subject has a decrease in a level of mutHTT protein encoded by said mutHTT allele as compared to a level of HTT protein of a control subject or a level of wild type HTT protein.

207. A method of ameliorating or delaying the onset of one or more symptoms of Huntington's disease (HD) in a subject in need thereof, wherein said method comprises:
a) selecting a subject comprising a mutant huntingtin (mutHTT) allele comprising:
i) at least 36 CAG repeats in exon 1; and
ii) a first single nucleotide polymorphism (SNP) allele in exon 50, wherein said SNP allele comprises a cytosine at a position corresponding to position 151 of SEQ ID NO: 2; and
b) administering by intrastriatal injection into said subject an AAV5 vector comprising:
i) a first nucleic acid molecule encoding an RNA-guided nuclease (RGN) polypeptide having the amino acid sequence of SEQ ID NO: 3; and
ii) a second nucleic acid molecule encoding a guide RNA having the nucleotide sequence of SEQ ID NO: 27 or 28; and
wherein 4 weeks post-administration of said AAV5 vector, said subject has a decrease in a level of mutHTT protein encoded by said mutHTT allele as compared to a level of HTT protein of a control subject or a level of wild type HTT protein.

208. The method of any one of claims 204-207, wherein said RGN polypeptide recognizes and cleaves and edits said mutHTT allele.

209. The method of any one of claims 204-208, wherein said editing comprises introducing an INDEL at or near said SNP.

210. The method of any one of claims 204-208, wherein said editing comprises introducing a premature stop codon at or near said SNP.

211. The method of any one of claims 204-210, wherein said mutHTT allele has at least 40 CAG repeats in exon 1.

212. The method of any one of claims 204-210, wherein said mutHTT allele has at least 56 CAG repeats in exon 1 and wherein said subject is younger than 18 years of age.

213. The method of any one of claims 204-212, wherein said administering occurs prior to onset of symptoms of Huntington's disease.

214. The method of any one of claims 204-213, wherein said method comprises preventing the onset of one or more symptoms of Huntington's disease.

215. The method of any one of claims 204-214, wherein said subject has at least one symptom of Huntington's disease.

216. The method of any of claims 204-215, wherein a decrease in a level of mutant HTT mRNA of at least 40% is observed as compared to a level of HTT mRNA of a control subject or a level of wild type HTT mRNA.

217. The method of any one of claims 204-216, wherein a decrease in the level of mutHTT protein is observed by 12 weeks after administration of said vector.

218. The method of claim 217, wherein at least a 40% decrease in the level of mutHTT protein is observed as compared to a level of HTT protein of a control subject or a level of wild type HTT protein.

219. The method of any of claims 204-218, wherein a decrease in a level of mutHTT mRNA is observed in at least 50% of the striatal cells in said subject.

220. The method of any of claims 204-219, wherein a decrease in the level of mutHTT protein is observed in at least 50% of the striatal cells in said subject.

221. The method of any of claims 204-220, wherein only the mutant HTT allele is edited.

222. A method for cleaving a mutant huntingtin (mutHTT) allele in a cell, wherein said mutHTT allele comprises a first single nucleotide polymorphism (SNP) allele in exon 50, wherein a protospacer adjacent motif (PAM) comprises said first SNP allele, wherein said method comprises introducing into the cell: (i) an RNA-guided nuclease (RGN) polypeptide or a nucleic acid molecule encoding said RGN polypeptide, and (ii) a guide RNA or a nucleic acid molecule encoding said guide RNA.

223. The method of claim 222, wherein said RGN polypeptide is capable of recognizing said PAM and cleaving said mutHTT allele.

224. The method of claim 223, wherein said mutHTT allele has at least 36 CAG repeats in exon 1.

225. The method of claim 223 or 224, wherein said cell has been assayed to determine whether said mutHTT allele comprises said first SNP allele prior to the introduction of (i) said RGN polypeptide or said nucleic acid molecule encoding said RGN polypeptide, and (ii) said guide RNA or said nucleic acid molecule encoding said guide RNA.

226. The method of any one of claims 223-225, wherein said cell comprises a wild-type HTT (wtHTT) allele comprising a second SNP allele where said PAM is not present, and said cell is thereby heterozygous for the SNP.

227. The method of claim 226, wherein said cell has been assayed to determine whether said cell is heterozygous for the SNP prior to the introduction of (i) said RGN polypeptide or said nucleic acid molecule encoding said RGN polypeptide, and (ii) said guide RNA or said nucleic acid molecule encoding said guide RNA.

228. The method of any one of claims 222-227, wherein said mutHTT allele is edited, thereby creating a genetically modified cell comprising said edited mutHTT allele.

229. The method of claim 228, wherein said editing comprises introducing an insertion and/or deletion (INDEL) at or near said SNP.

230. The method of claim 228, wherein said editing comprises introducing a premature codon at or near said SNP.

231. The method of any one of claims 228-230, wherein said genetically modified cell is a genetically modified stem cell.

232. The method of claim 231, wherein said genetically modified stem cell is a genetically modified induced pluripotent stem cell (iPSC) or a genetically modified mesenchymal stem cell (MSC).

233. The method of claim 232, wherein said method further comprises differentiating the genetically modified iPSC or MSC into a neuronal cell.

234. The method of any one of claims 228-233, wherein a level of mutHTT mRNA is reduced in said genetically modified cell as compared to a level of HTT mRNA in a non-genetically modified cell or to a level of wild type HTT mRNA.

235. The method of any one of claims 228-234, wherein a level of mutHTT protein encoded by said mutHTT allele is reduced in said genetically modified cell as compared to the level of HTT protein in a non-genetically modified cell or to the level of wild type HTT protein.

236. The method of any one of claims 228-235, further comprising selecting said genetically modified cell.

237. A genetically modified cell produced by the method of claim 236.

238. The method of any one of claims 222-227, wherein said introducing comprises administering a composition comprising (i) said RGN polypeptide or said nucleic acid molecule encoding said RGN polypeptide, and (ii) said guide RNA or said nucleic acid molecule encoding said guide RNA to a subject comprising said cell.

239. The method of claim 238, wherein the cell is a eukaryotic cell.

240. The method of claim 239, wherein the eukaryotic cell is a mammalian cell.

241. The method of claim 240, wherein the mammalian cell is a human cell.

242. The method of claim 240 or 241, wherein the mammalian cell or human cell is a stem cell.

243. The method of claim 240 or 241, wherein the mammalian cell or human cell is a forebrain neuron, a striatal neuron, a medium spiny neuron, a cortical neuron, or a glial cell.

244. The method of claim 240 or 241, wherein the mammalian cell or human cell is present in putamen, caudate, striatum, cerebral cortex, globus pallidus, hippocampus, amygdala, thalamus, hypothalamus, subthalamic nucleus, substantia nigra, cerebellum, brainstem, or a combination thereof.

245. A method for ameliorating or delaying the onset of one or more symptoms of Huntington's disease (HD) in a subject in need thereof, wherein said subject comprises a mutant huntingtin (mutHTT) allele comprising:
a) at least 36 CAG repeats in exon 1; and
b) a first single nucleotide polymorphism (SNP) allele in exon 50, wherein a protospacer adjacent motif (PAM) comprises said first SNP allele;
wherein said method comprises administering to said subject (i) an RNA-guided nuclease (RGN) polypeptide or a nucleic acid molecule encoding said RGN polypeptide, and (ii) a guide RNA or a nucleic acid molecule encoding said guide RNA, and wherein a level of a mutHTT protein encoded by said mutHTT allele is reduced as compared to a level of HTT protein in a control subject or a level of wild type HTT protein.

246. The method of claim 245, wherein said RGN polypeptide recognizes said PAM and cleaves and edits said mutHTT allele.

247. The method of claim 245, wherein said mutHTT allele has at least 40 CAG repeats in exon 1.

248. The method of claim 245, wherein said mutHTT allele has at least 56 CAG repeats in exon 1 and wherein said subject is younger than 18 years of age.

249. The method of any one of claims 245-248, wherein said administering occurs prior to onset of symptoms of Huntington's disease.

250. The method of any one of claims 245-249, wherein said method comprises preventing the onset of one or more symptoms of Huntington's disease.

251. The method of any one of claims 245-250, wherein said subject has at least one symptom of Huntington's disease.

252. The method of any one of claim 245-251, wherein said administering comprises intrastriatal, intraparenchymal, intrathecal, intracerebral, intracerebroventricular, intrathalamic, or intra-cisterna magna injection.

253. The method of any one of claims 245-252, wherein said subject has been assayed to determine whether said mutHTT allele comprising said PAM comprises said first SNP allele prior to the administration of (i) said RGN polypeptide or said nucleic acid molecule encoding said RGN polypeptide, and (ii) said guide RNA or said nucleic acid molecule encoding said guide RNA.

254. The method of any one of claims 245-253, wherein the subject comprises a wild-type HTT (wtHTT) allele comprising a second SNP allele where said PAM is not present, and said subject is thereby heterozygous for the SNP.

255. The method of claim 254, wherein said subject has been assayed to determine whether said subject is heterozygous for the SNP prior to the administration of (i) said RGN polypeptide or said nucleic acid molecule encoding said RGN polypeptide, and (ii) said guide RNA or said nucleic acid molecule encoding said guide RNA.

256. The method of any one of claims 245-255, wherein a level of mutHTT mRNA is reduced by at least 40% as compared to a level of HTT mRNA in a control subject or a level of wild type HTT mRNA.

257. The method of any one of claims 245-256, wherein a level of mutHTT protein is reduced by at least 40% as compared to a level of HTT protein in a control subject or a level of wild type HTT protein.

258. The method of any one of claims 245-257, wherein a decrease in a level of mutHTT protein is observed by 12 weeks after administration.

259. The method of any of claims 245-258, wherein a decrease in a level of mutHTT protein is observed in at least 50% of the striatal cells in said subject.

260. The method of any one of claims 222-259, wherein said PAM has a nucleotide sequence selected from the group consisting of: NNNNCC, NNRYA, NNGRR, and NNGG.

261. The method of claim 260, wherein the PAM sequence NNRYA comprises said first SNP allele, and wherein said first SNP allele is a thymine at a position corresponding to position 151 of SEQ ID NO: 1.

262. The method of claim 261, wherein said RGN polypeptide comprises an amino acid sequence having at least 90% sequence identity to SEQ ID NO: 7.

263. The method of claim 261 or 262, wherein said RGN polypeptide comprises the amino acid sequence of SEQ ID NO: 7.

264. The method of claim 261 or 262, wherein said guide RNA comprises a crRNA repeat having the nucleotide sequence of SEQ ID NO: 8 or 106 or a nucleotide sequence that differs from SEQ ID NO: 8 or 106 by 1 or 2 nucleotides and a tracrRNA having a nucleotide sequence having at least 90% sequence identity to SEQ ID NO: 9 or 107.

265. The method of claim 263, wherein said guide RNA comprises a crRNA repeat having the nucleotide sequence of SEQ ID NO: 8 or 106 and a tracrRNA having the nucleotide sequence of SEQ ID NO: 9 or 107.

266. The method of any one of claims 262-265, wherein said guide RNA comprises a spacer comprising a nucleotide sequence having complementarity with a target sequence having the nucleotide sequence of SEQ ID NO: 75 or 76.

267. The method of claim 266, wherein said spacer has the nucleotide sequence of SEQ ID NO: 80 or 81 or a nucleotide sequence that differs from SEQ ID NO: 80 or 81 by 1 or 2 nucleotides.

268. The method of claim 267, wherein said spacer has the nucleotide sequence of SEQ ID NO: 80 or 81.

269. The method of any one of claims 261-268, wherein said guide RNA is a single guide RNA.

270. The method of claim 269, wherein said single guide RNA has the nucleotide sequence of SEQ ID NO: 25 or 26.

271. The method of any one of claims 222-236 and 238-270, wherein said method comprises introducing a vector comprising said nucleic acid molecule encoding said RGN polypeptide, and said nucleic acid molecule encoding said guide RNA, and wherein said vector comprises: a truncated U6 promoter regulating the expression of a sgRNA; a CMVeb promoter regulating the expression of an RGN polypeptide; a c-Myc NLS at the N-terminus and C-terminus of said RGN polypeptide; an NLS linker protein connecting said c-Myc NLS to said RGN polypeptide; and an SV40 polyA tail.

272. The method of claim 271, wherein said truncated U6 promoter has the sequence set forth as SEQ ID NO: 128, said CMVeb promoter has the sequence set forth as SEQ ID NO: 90, said c-Myc NLS has the sequence set forth as SEQ ID NO: 125, said NLS linker protein has the sequence set forth as SEQ ID NO: 127, and said SV40 polyA tail has the sequence set forth as SEQ ID NO: 94.

273. The method of claim 271 or 272, wherein said sgRNA has the sequence set forth as SEQ ID NO: 26 and said nucleic acid molecule encoding said RGN polypeptide has the sequence set forth as SEQ ID NO: 88.

274. The method of any one of claims 271-273, wherein said vector comprises the sequence set forth as SEQ ID NO: 123.

275. The method of any one of claims 238-270, wherein said method comprises administering to said subject a vector comprising said nucleic acid molecule encoding said RGN polypeptide, and said nucleic acid molecule encoding said guide RNA, and wherein said vector comprises: a truncated U6 promoter regulating the expression of a sgRNA; a CMVeb promoter regulating the expression of an RGN polypeptide; a c-Myc NLS at the N-terminus and C-terminus of said RGN polypeptide; an NLS linker protein connecting said c-Myc NLS to said RGN polypeptide; and an SV40 polyA tail.

276. The method of claim 275, wherein said truncated U6 promoter has the sequence set forth as SEQ ID NO: 128, said CMVeb promoter has the sequence set forth as SEQ ID NO: 90, said c-Myc NLS has the sequence set forth as SEQ ID NO: 125, said NLS linker protein has the sequence set forth as SEQ ID NO: 127, and said SV40 polyA tail has the sequence set forth as SEQ ID NO: 94.

277. The method of claim 275 or 276, wherein said sgRNA has the sequence set forth as SEQ ID NO: 26 and said nucleic acid molecule encoding said RGN has the sequence set forth as SEQ ID NO: 88.

278. The method of any one of claims 275-277, wherein said vector comprises the sequence set forth as SEQ ID NO: 123.

279. The method of any one of claims 222-260, wherein the PAM sequence selected from the group consisting of: NNNNCC, NNGRR, and NNGG comprises said first SNP allele, and wherein said first SNP allele is a cytosine at a position corresponding to position 151 of SEQ ID NO: 1.

280. The method of claim 279, wherein said RGN polypeptide comprises an amino acid sequence having at least 90% sequence identity to any one of SEQ ID NOs: 3, 11, and 15.

281. The method of claim 279 or 280, wherein said RGN polypeptide comprises the amino acid sequence of any one of SEQ ID NOs: 3, 11, and 15.

282. The method of claim 279, wherein said RGN polypeptide and said guide RNA are selected from the group consisting of:
a) an RGN polypeptide comprising an amino acid sequence having at least 90% sequence identity to SEQ ID NO: 3 and a guide RNA comprising a crRNA repeat having the nucleotide sequence of SEQ ID NO: 4 or a nucleotide sequence that differs from SEQ ID NO: 4 by 1 or 2 nucleotides, and a tracrRNA having a nucleotide sequence having at least 90% sequence identity to SEQ ID NO: 5;
b) an RGN polypeptide comprising an amino acid sequence having at least 90% sequence identity to SEQ ID NO: 11 and a guide RNA comprising a crRNA repeat having the nucleotide sequence of SEQ ID NO: 12 or a nucleotide sequence that differs from SEQ ID NO: 12 by 1 or 2 nucleotides, and a tracrRNA having a nucleotide sequence having at least 90% sequence identity to SEQ ID NO: 13 or 120; and
c) an RGN polypeptide comprising an amino acid sequence having at least 90% sequence identity to SEQ ID NO: 15 and a guide RNA comprising a crRNA repeat having the nucleotide sequence of SEQ ID NO: 16 or a nucleotide sequence that differs from SEQ ID NO: 16 by 1 or 2 nucleotides, and a tracrRNA having a nucleotide sequence having at least 90% sequence identity to SEQ ID NO: 17.

283. The method of claim 282, wherein said RGN polypeptide and said guide RNA is selected from the group consisting of:
a) an RGN polypeptide comprising the amino acid sequence of SEQ ID NO: 3 and a guide RNA comprising a crRNA repeat having the nucleotide sequence of SEQ ID NO: 4 and a tracrRNA having the nucleotide sequence of SEQ ID NO: 5;
b) an RGN polypeptide comprising the amino acid sequence of SEQ ID NO: 11 and a guide RNA comprising a crRNA repeat having the nucleotide sequence of SEQ ID NO: 12 and a tracrRNA having the nucleotide sequence of SEQ ID NO: 13 or 120; and
c) an RGN polypeptide comprising the amino acid sequence of SEQ ID NO: 15 and a guide RNA comprising a crRNA repeat having the nucleotide sequence of SEQ ID NO: 16 and a tracrRNA having the nucleotide sequence of SEQ ID NO: 17.

284. The method of claim 282 or 283, wherein said RGN polypeptide and said guide RNA are the RGN polypeptide and guide RNA of claim 282(a) or 283(a), and wherein said guide RNA comprises a spacer having a nucleotide sequence having complementarity with a target sequence of SEQ ID NO: 77 or 78.

285. The method of claim 282 or 283, wherein said RGN polypeptide and said guide RNA are the RGN polypeptide and guide RNA of claim 282(a) or 283(a), and wherein said guide RNA comprises a spacer having the nucleotide sequence of SEQ ID NO: 82 or 83 or a nucleotide sequence that differs from SEQ ID NO: 82 or 83 by 1 or 2 nucleotides.

286. The method of claim 282 or 283, wherein said RGN polypeptide and said guide RNA are the RGN polypeptide and guide RNA of claim 282(a) or 283(a), and wherein said guide RNA comprises a spacer having the nucleotide sequence of SEQ ID NO: 82 or 83.

287. The method of any one of claims 279-286, wherein said guide RNA is a single guide RNA.

288. The method of claim 287, wherein said RGN polypeptide and said guide RNA are the RGN polypeptide and guide RNA of claim 282(a) or 283(a), and wherein said single guide RNA has the nucleotide sequence of SEQ ID NO: 27 or 28.

289. The method of any one of claims 222-288, wherein said PAM is present only on the mutHTT allele and not the wild-type HTT allele.

290. The method of any one of claims 222-289, wherein said nucleic acid molecule encoding said RGN polypeptide is an mRNA.

291. The method of any one of claims 222-289, wherein said nucleic acid molecule encoding said RGN polypeptide and said nucleic acid molecule encoding said guide RNA are in a viral vector.

292. The method of claim 291, wherein said viral vector is a lentiviral vector, a baculoviral vector, or an adeno-associated viral (AAV) vector.

293. The method of claim 292, wherein said AAV vector is AAV5.

294. A method for detecting mutant huntingtin (mutHTT) protein and wild type HTT (wtHTT) protein in a sample, said method comprising:
a) applying a sample that has been denatured to a capillary comprising a sieving medium;
b) applying a voltage differential to said capillary to separate proteins within said sample by molecular weight via electrophoresis;
c) immobilizing said separated proteins within said capillary;
d) applying to said capillary a first antibody or fragment thereof capable of binding to both mutHTT and wtHTT;
e) applying to said capillary a second antibody or fragment thereof capable of binding to said first antibody, wherein said second antibody comprises a detectable label; and
f) detecting said detectable label.

295. The method of claim 294, wherein said sieving medium is a hydrophilic polymer matrix.

296. The method of claim 294 or 295, wherein said sample is a biological sample.

297. The method any one of claims 294-296, wherein said detectable label is a chemiluminescent label or a fluorescent label.

298. The method of any one of claims 294-297, wherein said mutHTT and wtHTT protein in said sample are quantitated by comparison to a standard curve.

299. The method of any one of claims 294-298, wherein said method is capable of resolving the mutHTT protein from the wtHTT.

300. A method of ameliorating or delaying the onset of one or more symptoms of Huntington's disease (HD) in a subject in need thereof, wherein said method comprises delivering to said subject an adeno-associated viral (AAV) 5 vector comprising:
a) a guide RNA having a crRNA of SEQ ID NO: 8 or 106 and a tracrRNA of SEQ ID NO: 9 or 107; or
b) a single guide RNA having the nucleotide sequence of SEQ ID NO: 25 or 26.

301. The method of claim 300, wherein the subject comprises a mutant huntingtin (mutHTT) allele comprising:
a) at least 36 CAG repeats in exon 1; and
b) a first single nucleotide polymorphism (SNP) allele in exon 50, wherein said SNP allele comprises a thymine at a position corresponding to position 151 of SEQ ID NO: 1.

302. The method of claim 300 or 301, wherein said method comprises administering the AAV5 vector by intrastriatal injection.

303. The method of any of claims 300-302, wherein said subject has a decrease in a level of mutHTT protein encoded by said mutHTT allele as compared to a leve of HTT protein of a control subject or a level of wild type HTT protein.

304. The method of claim 303, wherein at least a 40% decrease in the level of mutHTT protein is observed as compared to a level of HTT protein of a control subject or a level of wild type HTT protein.

305. The method of claim 303 or 304, wherein a decrease in the level of mutHTT protein is observed by 4 weeks, 6 weeks, 8 weeks, 10 weeks, or 12 weeks after administration of said vector.

306. The method of any of claims 303-305, wherein a decrease in the level of mutHTT protein is observed in at least 50% of the striatal cells in said subject.

307. The method of any one of claims 300-306, wherein a level of mutHTT mRNA is reduced by at least 40% as compared to a level of HTT mRNA in a control subject or a level of wild type HTT mRNA.

308. The method of any one of claims 300-307, wherein said mutHTT allele has at least 40 CAG repeats in exon 1.

309. The method of any one of claims 300-308, wherein said mutHTT allele has at least 56 CAG repeats in exon 1 and wherein said subject is younger than 18 years of age.

310. The method of any one of claims 300-309, wherein said administering occurs prior to onset of symptoms of Huntington's disease.

311. The method of any one of claims 300-310, wherein said method comprises preventing the onset of one or more symptoms of Huntington's disease.

312. The method of any one of claims 300-311, wherein said subject has at least one symptom of Huntington's disease.

313. The method of any of claims 300-312, wherein only the mutant HTT allele is edited.


SEQUENCE LISTING (158 sequences)

SEQ ID NO: 1 - DNA, 178 bp, Homo sapiens
  source 1..178: mol_type = genomic DNA
gccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccg

SEQ ID NO: 2 - DNA, 178 bp, Homo sapiens
  source 1..178: mol_type = genomic DNA
gccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatccactgtgtgcacttcatcctggaggccg

SEQ ID NO: 3 - protein, 1071 aa, Bacillus sp.
MRELDYRIGLDIGTNSIGWGVIELSWNKDRERYEKVRIVDQGVRMFDRAEMPKTGASLAEPRRIARSSRRRLNRKSQRKKNIRNLLVQHGVITQEELDSLYPLSKKSMDIWGIRLDGLDRLLNHFEWARLLIHLAQRRGFKSNRKSELKDTETGKVLSSIQLNEKRLSLYRTVGEMWMKDPDFSKYDRKRNSPNEYVFSVSRAELEKEIVTLFAAQRRFQSPYASKDLQETYLQIWTHQLPFASGNAILNKVGYCSLLKGKERRIPKATYTFQYFSALDQVNRTRLGPDFQPFTKEQREIILNNMFQRTDYYKKKTIPEVTYYDIRKWLELDETIQFKGLNYDPNEELKKIEKKPFINLKAFYEINKVVANYSERTNETFSTLDYDGIGYALTVYKTDKDIRSYLKSSHNLPKRCYDDQLIEELLSLSYTKFGHLSLKAINHVLSIMQKGNTYKEAVDQLGYDTSGLKKEKRSKFLPPISDEITNPIVKRALTQARKVVNAIIRRHGSPHSVHIELARELSKNHDERTKIVSAQDENYKKNKGAISILSEHGILNPTGYDIVRYKLWKEQGERCAYSLKEIPADTFFNELKKERNGAPILEVDHILPYSQSFIDSYHNKVLVYSDENRKKGNRIPYTYFLETNKDWEAFERYVRSNKFFSKKKREYLLKRAYLPRESELIKERHLNDTRYASTFLKNFIEQNLQFKEAEDNPRKRRVQTVNGVITAHFRKRWGLEKDRQETYLHHAMDAIIVACTDHHMVTRVTEYYQIKESNKSVKKPYFPMPWEGFRDELLSHLASQPIAKKISEELKAGYQSLDYIFVSRMPKRSITGAAHKQTIMRKGGIDKKGKTIIIERLHLKDIKFDENGDFKMVGKEQDMATYEAIKQRYLEHGKNSKKAFETPLYKPSKKGTGNLIKRVKVEGQAKSFVREVNGGVAQNGDLVRVDLFEKDDKYYMVPIYVPDTVCSELPKKVVASSKGYEQWLTLDNSFTFKFSLYPYDLVRLVKGDEDRFLYFGTLDIDSDRLNFKDVNKPSKKNEYRYSLKTIEDLEKYEVGVLGDLRLVRKETRRNFH

SEQ ID NO: 4 - RNA, 21 nt, Bacillus sp.
  source 1..21: mol_type = genomic RNA
gtcatagttccattaaagcca

SEQ ID NO: 5 - RNA, 85 nt, Bacillus sp.
  source 1..85: mol_type = genomic RNA
tggctttgatgtttctatgataagggtttcgacccgtggcgtcggggatcgcctgcccattgaaatgggcttctccccatttatt

SEQ ID NO: 6 - RNA, 135 nt, synthetic construct
  source 1..135: mol_type = other RNA
  misc_difference 1..25: note = a, c, u, g, unknown or other
nnnnnnnnnnnnnnnnnnnnnnnnngtcatagttccattaaagccaaaagtggctttgatgtttctatgataagggtttcgacccgtggcgtcggggatcgcctgcccattgaaatgggcttctccccatttatt

SEQ ID NO: 7 - protein, 1150 aa, Enterococcus sp.
MYSIGLDLGISSVGWSVIDEETGKIVDLGVRLFSAKNSEKNLERRTSRGARRLIRRKTNRLKDAKKLLEAIGFYEDKALKNVCPYQLRVKGLTEGLTKGELYKVVLHIVKKRGISYLDEDDAEAAKESQDYKEQVRKNAQLLTKYTPGQIQLQRLKENNRVKTGINGQGHYQLNVFKVSAYADELATILKTQQALYPNELTDDWIALFVQPGIAENAGLIYRKRPYYHGPGNEANNSPYGRWSDFQKTGQPAANIFDKLIGKDFQGELRASGLSLSAQQYNLLNDLTNLKIDGEVSLSPEQKEFILTELMTKEFARFGVNDIAKLLGVKKEQLSGWRLDKKGKPEIHTLKGYRNWRKIFAEAGIDLATLPTETIDCLAKVLTLNTEREGVENTLAFELPELAEPVKSLVLDHYKELSQSISTQAWHRFSLKTLHLLIPELIKSTSEQNTLLEQFQLKAGVRKRYSDYKKLPTKEVLAEIYNPTVNKTVSQAFKVMDALLEKYGKDQIHYITVEMPRDDNEEEERKRIKELQTKNSQRKNDSQQYFLQKSGWSQEKFQATIHKNRRFLAKLLYYFEQDGVCAYTGNPISPELLVSDSTEIDHIIPISISLDDSINNKVLVLSHANQVKGQQTPYDARMAGAFNKINGKFSNWDEYQKWVESRPFSRKKVNNLLETRNIFDSEQVQKFLSRNLNDTRYASRLVLNTLQSFFENQDTIVRVVNGSFTHTLRKKWGADLDKTRETHHHHAVDATLCAVTPFVKVSRYHYAVNEETGEKFMREIDVETGEILDEIPYREYKKAKHYERKTYQVKWSNFREQLKPITIHPKIKFSHQVDRKANRKLSDATIYSVREKTEVKTLKSGKEKITTDEYTIGKIKDIYTVDGWEAFKKKQDKLLMKEFDEKTYELLVTIAATTPDFQEVEEKNGKVKRVKRSPFAVYCEENGIPAIRKYAKKNNGPVIRSLKYYDGKLNKHINITKDEKGRPVEQTKNGRKVTLQSLKPYRYDIYQDLETKAYYTVQLYYSDLRFVEGEYGITEKEYMKKVAEQTKGQVVRFCFSLQKNDGLEIEWKDSQRYDVRFYNFQSANSINFKGLEQEMIPAENQFKQKPYNNGAINLNIAKYGKEGKKLRKFNTDILGKKHHLSYEKEPKNIIK

SEQ ID NO: 8 - RNA, 19 nt, Enterococcus sp.
  source 1..19: mol_type = genomic RNA
gttattgtactctcaataa

SEQ ID NO: 9 - RNA, 67 nt, Enterococcus sp.
  source 1..67: mol_type = genomic RNA
ttattgagaatctacaataataaggcatcttgccgaatttaccgccctacatatgtagggcggtttt

SEQ ID NO: 10 - RNA, 119 nt, synthetic construct
  source 1..119: mol_type = other RNA
  misc_difference 1..29: note = a, c, u, g, unknown or other
nnnnnnnnnnnnnnnnnnnnnnnnnnnnngttattgtactctcaataaaaagttattgagaatctacaataataaggcatcttgccgaatttaccgccctacatatgtagggcggtttt

SEQ ID NO: 11 - protein, 1052 aa, Staphylococcus sp.
MVTKYILGLDIGITSVGYGIINYEDKTIIDAGVRLFPEANVENNEGRRSKRGARRLKRRRIHRLDRIKQLLSEYNLVDLDNIPQSPSPYEIRVKGLREELSKDELVIALLHIAKRRGIHNVEAVDETQDEGNELSTKEQLAKNNNLLKDKYVCELLLERLKDGKVRGEKNRFKTTDIIKEVKQLLETQKEAHQLDDDFINRYIDLIETRREYFEGPGKGSPFGWGGDLKKWYETLMGHCTYFPNELRSVKYSYSADLFNALNDLNNLVIQREGNSKLEYHEKYHIIENVFKQKKKPTLKQIANEIGVSPDDIKGFRINKSGKETFTEFKLYHDLKKVLIDQSILENVQLLDQIAEILTIYQDKESIINELNQLSEIINEQDKESISNLSGYNGTHRLSLKCINLVIEELWHTSRNQMEIFTYLNIKPKKIDLAKTNKIPKNMIDEFILSPVVKRTFGQAINVINKVIEKYGVPEDIIIELARESNSKDKQKFINSLQKKNETTRKRINEIIGQYGNQNAKRLVEKIRLHDEQEGKCLYSLESIPLEDLINNPQYYEVDHIIPRSVSFDNSYQNKVLVKQTENSKKSNRTPYQYFNSGETKLSYNQFKQHVLNLSKSKDRISKKKKEYLLEERDINKYEVQKEFINRNLVDTRYATRELTNYLKAYFSANDMDVKVKTINGSFTDYLRKVWKFKKERNHGYKHHAEDALIIANADFLFKENKKLKKANAILEQPSLDNGKSDATVENDNEYVETFSIPKQVNDIKEFRDFKFSHRVDKKPNRQLINDTLYSTRKIENHTFIVSPITNIYSKDNDELKKKFNKNPEKFLMYQHDPKTFEKLEVIMKQYANEKNPLAKYHEETGEYLTKYSKKNNGPIVKTIKVLGDKVGKHLDVTHKYKYSNSKIVKKTINPYRFDVYLTDKGYKFITISYLDVLKKDNYYYILKEKYEELKIKKSISDTDQFIGSFYYNDLIKINDQIFKVVGVNNDLLNRIELDLLDISYKEYCKINNIKTNRIIKGITKKITNIEKFSTDVLGNLYKAHSNHPQLIFKQRD

SEQ ID NO: 12 - RNA, 17 nt, Staphylococcus sp.
  source 1..17: mol_type = genomic RNA
gttttagtactctgtaa

SEQ ID NO: 13 - RNA, 64 nt, Staphylococcus sp.
  source 1..64: mol_type = genomic RNA
ttacagaatctactaaaacaagactatatatcgtgtttatcccatcaatttattgatgggattt

SEQ ID NO: 14 - RNA, 114 nt, synthetic construct
  source 1..114: mol_type = other RNA
  misc_difference 1..29: note = a, c, u, g, unknown or other
nnnnnnnnnnnnnnnnnnnnnnnnnnnnngttttagtactctgtaaaaagttacagaatctactaaaacaagactatatatcgtgtttatcccatcaatttattgatgggattt

SEQ ID NO: 15 - protein, 1130 aa, unidentified
MSKVAKNMTRVNLGFDIGIASVGWSVLDNQTGKILETGVSIFPSGSASRNEERRSFRQSRRLIRRKKARICDMDHLLKKNGFPFPGNTGANPYEIRVKGLTEKLSREELAIALHHLVKRRGISYDLKDVEDESAGGTNYQESIAVNQRLLKKETPAEIQLARLTECGKVRGQVKSLGEDNTATTLLNVFPNAAYQEEMVKLLKKQQEFYGEIDDPFMETAIGILSRKREYFIGPGSEKSRTDYGIYRTDGTTLKNLFEILVGKDKIFPDQYRAAGNSYTAQLYNLLNDLNNLEVDATEDGKLTTAHKEQIIEELTTTTGNVNMLKLIAKVAGTSPAGIKKYRVDREGKPEFHSLAIYRRLRKKLGEAGFEINEWPPEFFDDYGPIVTLNTESGELRKWLAEEGSRKYDFLTEPVIEAILANKSAFDSVGKNKWHRFSLKTMQLLIPELLHTFKEQMTILAEMGLLHENKKDYGDQNKVDVKYLTENLYNPVVRKSVKQAMDIFNALFEKYANIDYVVIEMPRDDAEDELEQKKQFQKFQLKNEKEKDASLKEFQELAGVSDLQLEAQLRKRKKLRQKIRMWYQQRGKCPYSGKTIAAVDLFHQDNQFEIDHIIPLSVSFDDGQNNKVLCYSEMNQEKGKQTPYAFMSRGGGQGFSALQAYVKSNNRLENAKKRNLLFTEDINDLEVRKRFIARNLVDTRYASRIVLNELQQFVRSKELDTRVTVIRGKLTSKLRDRWRLNKSRETHHHHAVDAAVIAVSPMLKMWEKNAEIIPLKVDENTVDLKSGEIITDQEYAAQMYELPYARFLEQMPELHKKIKFHHQVDKKMNRKVSDATLYSTRKAKVGTDKKEQEYVIGKIKDIYQFDQYKKFKKLYDGDKSKFLMQRLDPQTFAKLEKIMEDYPAKIDATQPNGTIKLVDISPFELYRREHGPVTKYAKKNNGPAIKSLKFYDSIVGSSVKITPKNAKGKEVILKSLKPWRTDVYYNHEKEQYEIMGIKYADLKFKGDNYGITKARYQEIKEEEGVSEESEFLFSLYRGDRIQVSNGEDKIDLLFLSRSNPAKKGYVELKPIDRNQLNGKEVVSVYGAASGGRLKKQFVKKNHTLHKVNTDILGNPFYIKKESDQPKNILDL

SEQ ID NO: 16 - RNA, 24 nt, unidentified
  source 1..24: mol_type = unassigned RNA
gttattgtactctcaaggaatcag

SEQ ID NO: 17 - RNA, 69 nt, unidentified
  source 1..69: mol_type = unassigned RNA
ctgattccttgagaatctacaataataaggctttatgccgaaattcccactcttaacggagtgggtttt

SEQ ID NO: 18 - RNA, 120 nt, synthetic construct
  source 1..120: mol_type = other RNA
  misc_difference 1..23: note = a, c, u, g, unknown or other
nnnnnnnnnnnnnnnnnnnnnnngttattgtactctcaaggaatcagaaagctgattccttgagaatctacaataataaggctttatgccgaaattcccactcttaacggagtgggtttt

SEQ ID NO: 19 - DNA, 20 bp, synthetic construct
  source 1..20: mol_type = other DNA
aaacgaaggtacacgagtgg

SEQ ID NO: 20 - DNA, 20 bp, synthetic construct
  source 1..20: mol_type = other DNA
gcaaacaggcagcacaaaat

SEQ ID NO: 21 - DNA, 31 bp, synthetic construct
  source 1..31: mol_type = other DNA
agctgatgggcgccttcgagtccctcaagtc

SEQ ID NO: 22 - DNA, 34 bp, synthetic construct
  source 1..34: mol_type = other DNA
tgcactgcaggcgcctccaggatgaagtgcacac

SEQ ID NO: 23 - DNA, 25 bp, synthetic construct
  source 1..25: mol_type = other DNA
cagatcccgctgagtctggatctcc

SEQ ID NO: 24 - DNA, 20 bp, synthetic construct
  source 1..20: mol_type = other DNA
cggctgaggcagcagcggct

SEQ ID NO: 25 - RNA, 115 nt, synthetic construct
  source 1..115: mol_type = other RNA
accggcctccaggatgaagtgcacagttattgtactctcaataaaaagttattgagaatctacaataataaggcatcttgccgaatttaccgccctacatatgtagggcggtttt

SEQ ID NO: 26 - RNA, 101 nt, synthetic construct
  source 1..101: mol_type = other RNA
accggcctccaggatgaagtgcacagttattgtactcaaaggaatctacaataataaggcatcttgccgaatttaccgccctacatatgtagggcggtttt

SEQ ID NO: 27 - RNA, 135 nt, synthetic construct
  source 1..135: mol_type = other RNA
gagtttgtgacccacgcctgctcccgtcatagttccattaaagccaaaagtggctttgatgtttctatgataagggtttcgacccgtggcgtcggggatcgcctgcccattgaaatgggcttctccccatttatt

SEQ ID NO: 28 - RNA, 132 nt, synthetic construct
  source 1..132: mol_type = other RNA
tttgtgacccacgcctgctcccgtcatagttccattaaagccaaaagtggctttgatgtttctatgataagggtttcgacccgtggcgtcggggatcgcctgcccattgaaatgggcttctccccatttatt

SEQ ID NO: 29 - RNA, 110 nt, synthetic construct
  source 1..110: mol_type = other RNA
cggcctccaggatgaagtgcacacagttttagtactctgtaaaaagttacagaatctactaaaacaaggcaaaatgccgtgtttatctcgtcaacttgttggcgagattt

SEQ ID NO: 30 - DNA, 6849 bp, synthetic construct
  source 1..6849: mol_type = other DNA
gtgcacgatatcgggtccccaattgctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgtcgacaatgcacgcgttcgaagtactcatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagtttgtgacccacgcctgctcccgtcatagttccattaaagccaaaagtggctttgatgtttctatgataagggtttcgacccgtggcgtcggggatcgcctgcccattgaaatgggcttctccccatttatttttttgagctcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagcggccgcgccaccatggcccctaagaagaaaagaaaggtgatgagagagctggactacagaattggcctggacatcggcaccaacagcatcggatggggcgtgatcgagctgtcctggaacaaagaccgggagagatacgagaaggtcagaatcgtggatcaaggcgtgagaatgttcgacagagccgagatgcccaagacaggcgccagcttagctgaacccagaagaatcgccagatccagcagacggagactgaatcgcaagtcccagagaaagaaaaacatccggaacctgctggtgcaacacggcgtgatcacacaggaggaactggatagcctgtaccccctgagcaaaaagagcatggacatctggggcattcggctcgacggcctggacagactcctcaatcatttcgagtgggccagactgctgatccacctggctcagagacggggctttaagtccaacagaaagagtgaactgaaagatacagagacaggcaaggtgctgagcagcatccaactgaacgagaaacggctgagcttgtatagaaccgtgggcgagatgtggatgaaggaccccgacttctctaaatacgataggaagagaaatagccccaacgaatacgtgttcagcgtgtctagagccgagctggaaaaggaaatcgtgaccctgttcgccgcccagcggagattccagagcccttacgccagcaaagatctgcaggagacatatctgcagatctggacccaccaactgcctttcgccagcggcaatgccatcctgaacaaggtcggatactgctccctgttgaaaggcaaagaaagaaggattcccaaggctacatacaccttccaatacttctctgctctggaccaggtgaatcggaccagactgggacctgatttccagcccttcaccaaggagcaacgggaaattatcttgaacaacatgttccagaggacagattactacaagaagaaaaccatccccgaggtgacctactatgacatacggaagtggctggaattggacgagacaattcagttcaagggcctgaactacgaccctaacgaggaactgaagaagatcgagaagaagccttttatcaatctgaaggccttctacgagatcaacaaggtggtggccaactacagcgaaagaaccaacgagaccttctccaccctggactacgacggcatcggctacgccctgaccgtgtacaaaaccgacaaggatatccgcagctacctgaagagcagtcacaacctacctaagagatgctacgacgaccaactgatcgaggaactgctgagcctgagctacacaaagttcggccacctgtccctgaaagccatcaaccacgtgctgtctatcatgcagaagggcaatacctacaaggaagccgtggaccaactgggctacgacaccagcggccttaagaaggagaagaggtccaagttcctgccacctatttctgatgaaatcacgaatccaatcgtgaaaagggccctgacccaggccagaaaagtggtgaacgccataattagaagacacggatctcctcactccgtgcacatcgagctggccagagagctgagcaagaaccacgacgagcggacaaagatcgtcagcgcccaggatgaaaactacaagaaaaacaagggcgctatcagcatcctgtctgagcacggcatcctgaaccctacaggctacgacatcgtgagatacaaactgtggaaggagcagggcgaacggtgcgcctacagcctgaaggaaatccctgccgatacatttttcaacgagctgaagaaggaacgcaacggcgcccctatccttgaagtggaccacatcctgccctacagccagtccttcatcgactcctaccacaacaaggtcctggtgtacagcgacgaaaaccggaaaaagggcaacagaatcccttatacctacttcctggaaaccaacaaggattgggaggcctttgagcggtacgtgcggagcaacaaattcttctccaagaaaaagcgagagtaccttctgaagcgggcttatctgcctagagaatctgagctgatcaaagaacgccacctgaacgacaccagatacgcctctaccttcctgaagaacttcatcgagcagaacctgcagttcaaggaagccgaggacaaccccagaaaaagacgggtgcaaaccgtgaacggcgttatcaccgcccacttcagaaagcggtggggcctggagaaggaccggcaggagacatacctccatcacgctatggacgccatcatcgtggcttgtacagaccaccacatggtcaccagagtgaccgagtactatcagatcaaggaaagcaacaagagcgtgaagaagccctattttcctatgccttgggaaggcttccgggacgagctgctgagccacttggcttctcagcctatcgccaagaaaatcagcgaggaactgaaggccggctaccagagcctggactacatcttcgtgtccagaatgcctaagagaagcattacaggcgctgctcataagcagaccatcatgcggaagggaggaattgacaagaagggcaaaacaatcatcatcgaacggctgcacctgaaggatatcaagttcgacgagaacggagatttcaagatggtgggcaaggaacaggacatggccacatacgaagctattaaacagagatacctggagcacggcaagaatagcaagaaggccttcgagacccctctgtacaagcccagcaaaaagggcacaggtaacctgatcaagcgggtgaaggtggaaggacaggccaagagctttgtgagggaagtgaacggcggagtggcccaaaatggcgatctggttagagttgatttgtttgagaaggatgataagtactacatggtccccatctacgtgccagacaccgtgtgtagcgagctgcccaaaaaggtggtcgccagctctaagggctatgagcagtggctgacactggataacagcttcacctttaagttcagcctgtacccttatgatctggtgcggctggtcaagggagatgaggatcggttcctgtactttggcaccctggacatcgacagcgacagacttaacttcaaggacgtgaacaagccaagcaagaagaacgagtaccggtacagcttgaaaaccatcgaggacttggagaagtacgaggtgggcgtgctgggcgatctaagactggtccggaaggaaactcgaagaaacttccacagcggcggaagcaaaagacctgccgctacaaagaaggccggccaggccaagaaaaagaagtaagctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaagcttggtaggaacccctagtgatggagttggccactccctctctgcgcgctcgctcgctcactgaggccgggcgaccaaaggtcgcccgacgcccgggctttgcccgggcggcctcagtgagcgagcgagcgcgcagaggcatttaattaagcaagctgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtcc

SEQ ID NO: 31 - DNA, 6847 bp, synthetic construct
  source 1..6847: mol_type = other DNA
gtgcacgatatcgggtccccaattgctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgtcgacaatgcacgcgttcgaagtactcatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtttgtgacccacgcctgctcccgtcatagttccattaaagccaaaagtggctttgatgtttctatgataagggtttcgacccgtggcgtcggggatcgcctgcccattgaaatgggcttctccccatttatttttttgagctcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagcggccgcgccaccatggcccctaagaagaaaagaaaggtgatgagagagctggactacagaattggcctggacatcggcaccaacagcatcggatggggcgtgatcgagctgtcctggaacaaagaccgggagagatacgagaaggtcagaatcgtggatcaaggcgtgagaatgttcgacagagccgagatgcccaagacaggcgccagcttagctgaacccagaagaatcgccagatccagcagacggagactgaatcgcaagtcccagagaaagaaaaacatccggaacctgctggtgcaacacggcgtgatcacacaggaggaactggatagcctgtaccccctgagcaaaaagagcatggacatctggggcattcggctcgacggcctggacagactcctcaatcatttcgagtgggccagactgctgatccacctggctcagagacggggctttaagtccaacagaaagagtgaactgaaagatacagagacaggcaaggtgctgagcagcatccaactgaacgagaaacggctgagcttgtatagaaccgtgggcgagatgtggatgaaggaccccgacttctctaaatacgataggaagagaaatagccccaacgaatacgtgttcagcgtgtctagagccgagctggaaaaggaaatcgtgaccctgttcgccgcccagcggagattccagagcccttacgccagcaaagatctgcaggagacatatctgcagatctggacccaccaactgcctttcgccagcggcaatgccatcctgaacaaggtcggatactgctccctgttgaaaggcaaagaaagaaggattcccaaggctacatacaccttccaatacttctctgctctggaccaggtgaatcggaccagactgggacctgatttccagcccttcaccaaggagcaacgggaaattatcttgaacaacatgttccagaggacagattactacaagaagaaaaccatccccgaggtgacctactatgacatacggaagtggctggaattggacgagacaattcagttcaagggcctgaactacgaccctaacgaggaactgaagaagatcgagaagaagccttttatcaatctgaaggccttctacgagatcaacaaggtggtggccaactacagcgaaagaaccaacgagaccttctccaccctggactacgacggcatcggctacgccctgaccgtgtacaaaaccgacaaggatatccgcagctacctgaagagcagtcacaacctacctaagagatgctacgacgaccaactgatcgaggaactgctgagcctgagctacacaaagttcggccacctgtccctgaaagccatcaaccacgtgctgtctatcatgcagaagggcaatacctacaaggaagccgtggaccaactgggctacgacaccagcggccttaagaaggagaagaggtccaagttcctgccacctatttctgatgaaatcacgaatccaatcgtgaaaagggccctgacccaggccagaaaagtggtgaacgccataattagaagacacggatctcctcactccgtgcacatcgagctggccagagagctgagcaagaaccacgacgagcggacaaagatcgtcagcgcccaggatgaaaactacaagaaaaacaagggcgctatcagcatcctgtctgagcacggcatcctgaaccctacaggctacgacatcgtgagatacaaactgtggaaggagcagggcgaacggtgcgcctacagcctgaaggaaatccctgccgatacatttttcaacgagctgaagaaggaacgcaacggcgcccctatccttgaagtggaccacatcctgccctacagccagtccttcatcgactcctaccacaacaaggtcctggtgtacagcgacgaaaaccggaaaaagggcaacagaatcccttatacctacttcctggaaaccaacaaggattgggaggcctttgagcggtacgtgcggagcaacaaattcttctccaagaaaaagcgagagtaccttctgaagcgggcttatctgcctagagaatctgagctgatcaaagaacgccacctgaacgacaccagatacgcctctaccttcctgaagaacttcatcgagcagaacctgcagttcaaggaagccgaggacaaccccagaaaaagacgggtgcaaaccgtgaacggcgttatcaccgcccacttcagaaagcggtggggcctggagaaggaccggcaggagacatacctccatcacgctatggacgccatcatcgtggcttgtacagaccaccacatggtcaccagagtgaccgagtactatcagatcaaggaaagcaacaagagcgtgaagaagccctattttcctatgccttgggaaggcttccgggacgagctgctgagccacttggcttctcagcctatcgccaagaaaatcagcgaggaactgaaggccggctaccagagcctggactacatcttcgtgtccagaatgcctaagagaagcattacaggcgctgctcataagcagaccatcatgcggaagggaggaattgacaagaagggcaaaacaatcatcatcgaacggctgcacctgaaggatatcaagttcgacgagaacggagatttcaagatggtgggcaaggaacaggacatggccacatacgaagctattaaacagagatacctggagcacggcaagaatagcaagaaggccttcgagacccctctgtacaagcccagcaaaaagggcacaggtaacctgatcaagcgggtgaaggtggaaggacaggccaagagctttgtgagggaagtgaacggcggagtggcccaaaatggcgatctggttagagttgatttgtttgagaaggatgataagtactacatggtccccatctacgtgccagacaccgtgtgtagcgagctgcccaaaaaggtggtcgccagctctaagggctatgagcagtggctgacactggataacagcttcacctttaagttcagcctgtacccttatgatctggtgcggctggtcaagggagatgaggatcggttcctgtactttggcaccctggacatcgacagcgacagacttaacttcaaggacgtgaacaagccaagcaagaagaacgagtaccggtacagcttgaaaaccatcgaggacttggagaagtacgaggtgggcgtgctgggcgatctaagactggtccggaaggaaactcgaagaaacttccacagcggcggaagcaaaagacctgccgctacaaagaaggccggccaggccaagaaaaagaagtaagctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaagcttggtaggaacccctagtgatggagttggccactccctctctgcgcgctcgctcgctcactgaggccgggcgaccaaaggtcgcccgacgcccgggctttgcccgggcggcctcagtgagcgagcgagcgcgcagaggcatttaattaagcaagctgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtcc

SEQ ID NO: 32 - DNA, 7067 bp, synthetic construct
  source 1..7067: mol_type = other DNA
gtgcacgatatcgggtccccaattgctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgtcgacaatgcacgcgttcgaagtactcatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccggcctccaggatgaagtgcacagttattgtactcaaaggaatctacaataataaggcatcttgccgaatttaccgccctacatatgtagggcggtttttttttgtctagacaagttggagctcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagcggccgcgccaccatggcccctaagaagaaaagaaaggtgatgtacagtatagggttagatttagggataagtagtgttggttggtcagttattgatgaagaaacaggaaaaattgtggacttaggtgttcgcttatttagtgcaaaaaatagtgagaaaaatttagaacgacgaactagtcgtggtgctcgtcgcttaatacgacggaaaacaaatcgcttaaaagatgcgaagaaattacttgaagcgattggcttttatgaagacaaggcattaaaaaatgtttgtccttatcaattaagagtaaaaggtttgactgagggcttaaccaagggagagctttacaaagttgtgcttcatattgtcaaaaaacgtgggattagttatttggatgaggatgatgctgaagccgcaaaagaaagccaagattacaaagaacaagtcagaaaaaatgctcaattgttaacaaagtatacgcctggtcaaattcaattgcagcgactgaaagaaaacaatcgagtgaaaacagggattaatggtcaaggccattatcagttaaatgtctttaaagtttctgcttatgctgatgaattggctaccatcctgaaaactcaacaggccttgtatccaaatgaattaacagatgattggattgcgttatttgtgcaaccaggtattgcagagaatgccgggttaatttatcgcaaacgcccttattatcatggaccaggaaatgaagctaataatagcccttatggtcgttggtctgatttccaaaaaacaggacaaccggcagcaaatatttttgataaattgattggcaaagactttcaaggtgaactacgagcaagcggcttaagtttatctgcacaacaatataatttactgaatgatttaaccaatttaaaaattgatggggaagtttcactttctcctgaacaaaaggaattcattttaacagaactaatgacgaaagaatttgctcgttttggtgtcaacgatatcgctaaattattaggtgtaaaaaaagagcaactcagcggctggcgattggataaaaaaggtaaaccagaaattcatacattaaaaggctatcgcaactggcggaaaatttttgcggaagcagggatcgatttggctactttaccaacagaaacgattgactgtttagcgaaagtgctaacattgaataccgaacgcgaaggggttgaaaacacattagcttttgaacttcctgagttagcagaaccggtgaagtcactggttttggatcattacaaagaattaagtcaaagtattagcacgcaagcatggcaccgtttttctctaaaaacgttacacctattaattccagaactaatcaaatccacatcggaacaaaatacgctcttagaacagtttcagttaaaagcaggtgtcagaaaacgctacagtgattataaaaagttaccaaccaaagaggttctagcagaaatttataatcctacagtgaacaaaacagtcagtcaagccttcaaagttatggatgctttgttggaaaaatatggcaaagaccaaatccattatattaccgtcgaaatgccacgtgatgataacgaagaagaagaacgaaaacggataaaagagctacaaacaaagaatagtcaacgaaaaaatgacagtcaacagtactttctacaaaagtcaggttggagtcaagaaaaatttcaagcaaccattcacaaaaatcgtcgttttttagccaaattgttatactactttgaacaagacggggtatgtgcctatacgggaaatcctatttcacctgaattattagtcagtgattcaacagaaattgaccatattattccaatttcgataagtctagatgattctattaataataaagtcttggttttaagtcacgcgaatcaagtcaaaggacaacaaacaccttatgatgctaggatggctggcgcatttaacaaaattaatgggaaatttagcaactgggatgagtatcaaaagtgggtagaaagtcgtccgttcagtcgtaaaaaagtaaataatttattggaaacacggaatatttttgatagtgaacaagttcaaaagtttctgtcacgaaatcttaacgatacgcgttacgctagtcgtttggttttaaatacgttgcaaagcttttttgaaaatcaagacacaattgttcgagtagtaaatggtagtttcactcataccttacgtaaaaaatggggtgctgacttagataaaactcgtgaaacccatcatcatcatgccgttgatgcgacactttgtgcagtgacaccatttgtgaaagtcagtcgttatcactatgcggtcaacgaagaaaccggtgaaaagttcatgcgtgaaattgatgttgaaactggtgaaattctcgatgaaattccgtatagagaatacaaaaaagcaaaacattatgaacgaaaaacgtaccaagtcaaatggtcaaattttcgggaacaattaaaaccaataacgattcatccaaaaattaaattttctcatcaagtggatcgcaaagctaatcgcaaactcagtgatgcaaccatttactctgttcgtgaaaaaacagaagtcaaaacactaaaaagtggtaaagaaaaaataacgacagatgaatataccatcggaaaaattaaagatatttacacggtagatggttgggaagcatttaagaaaaaacaagacaaactattaatgaaagagtttgatgaaaaaacgtacgaattattagtgaccattgcagcaacaacgccagattttcaagaagtggaagaaaaaaatggcaaggtgaaacgtgtgaagcgctcaccatttgctgtttactgcgaagaaaatgggattccagcgattcgaaaatatgcgaaaaaaaataacggtccagttatccgtagcttaaaatattacgatggcaaattaaacaaacatatcaatattaccaaagacgaaaaaggtcgaccagttgaacaaaccaaaaatggtcgcaaagttactttacaaagtctaaaaccctatcgttacgatatttatcaagatttagaaacgaaggcttactatacagtgcaactatattattctgacttgcgttttgtggaaggtgagtatggaattacagaaaaagaatacatgaagaaagtcgctgagcagacaaagggacaagtcgtacgtttctgcttctctttacaaaaaaatgatggtttggaaatcgaatggaaagatagccaacgctatgatgtgcgtttttataatttccaaagtgctaatagcattaactttaaagggttagaacaagaaatgataccagcagaaaatcaatttaagcaaaaaccttataataatggagccatcaatttaaatattgctaaatatggcaaagaaggcaaaaagttacgaaaattcaatacagatattttaggaaagaaacatcatctatcttatgaaaaagaaccaaagaatattattaaaagcggcggaagcaaaagacctgccgctacaaagaaggccggccaggccaagaaaaagaagtaagctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaagcttggtaggaacccctagtgatggagttggccactccctctctgcgcgctcgctcgctcactgaggccgggcgaccaaaggtcgcccgacgcccgggctttgcccgggcggcctcagtgagcgagcgagcgcgcagaggcatttaattaagcaagctgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtcc

SEQ ID NO: 33 - DNA, 7344 bp, synthetic construct
  source 1..7344: mol_type = other DNA
gtgcacgatatcgggtccccaattgctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgtcgacaatgcacgcgttcgaagtactcatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccggcctccaggatgaagtgcacagttattgtactcaaaggaatctacaataataaggcatcttgccgaatttaccgccctacatatgtagggcggtttttttttgtctagacaagttggagctcagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccccgggtaccggtcgccaccatggcccctaagaagaaaagaaaggtgatgtacagtatagggttagatttagggataagtagtgttggttggtcagttattgatgaagaaacaggaaaaattgtggacttaggtgttcgcttatttagtgcaaaaaatagtgagaaaaatttagaacgacgaactagtcgtggtgctcgtcgcttaatacgacggaaaacaaatcgcttaaaagatgcgaagaaattacttgaagcgattggcttttatgaagacaaggcattaaaaaatgtttgtccttatcaattaagagtaaaaggtttgactgagggcttaaccaagggagagctttacaaagttgtgcttcatattgtcaaaaaacgtgggattagttatttggatgaggatgatgctgaagccgcaaaagaaagccaagattacaaagaacaagtcagaaaaaatgctcaattgttaacaaagtatacgcctggtcaaattcaattgcagcgactgaaagaaaacaatcgagtgaaaacagggattaatggtcaaggccattatcagttaaatgtctttaaagtttctgcttatgctgatgaattggctaccatcctgaaaactcaacaggccttgtatccaaatgaattaacagatgattggattgcgttatttgtgcaaccaggtattgcagagaatgccgggttaatttatcgcaaacgcccttattatcatggaccaggaaatgaagctaataatagcccttatggtcgttggtctgatttccaaaaaacaggacaaccggcagcaaatatttttgataaattgattggcaaagactttcaaggtgaactacgagcaagcggcttaagtttatctgcacaacaatataatttactgaatgatttaaccaatttaaaaattgatggggaagtttcactttctcctgaacaaaaggaattcattttaacagaactaatgacgaaagaatttgctcgttttggtgtcaacgatatcgctaaattattaggtgtaaaaaaagagcaactcagcggctggcgattggataaaaaaggtaaaccagaaattcatacattaaaaggctatcgcaactggcggaaaatttttgcggaagcagggatcgatttggctactttaccaacagaaacgattgactgtttagcgaaagtgctaacattgaataccgaacgcgaaggggttgaaaacacattagcttttgaacttcctgagttagcagaaccggtgaagtcactggttttggatcattacaaagaattaagtcaaagtattagcacgcaagcatggcaccgtttttctctaaaaacgttacacctattaattccagaactaatcaaatccacatcggaacaaaatacgctcttagaacagtttcagttaaaagcaggtgtcagaaaacgctacagtgattataaaaagttaccaaccaaagaggttctagcagaaatttataatcctacagtgaacaaaacagtcagtcaagccttcaaagttatggatgctttgttggaaaaatatggcaaagaccaaatccattatattaccgtcgaaatgccacgtgatgataacgaagaagaagaacgaaaacggataaaagagctacaaacaaagaatagtcaacgaaaaaatgacagtcaacagtactttctacaaaagtcaggttggagtcaagaaaaatttcaagcaaccattcacaaaaatcgtcgttttttagccaaattgttatactactttgaacaagacggggtatgtgcctatacgggaaatcctatttcacctgaattattagtcagtgattcaacagaaattgaccatattattccaatttcgataagtctagatgattctattaataataaagtcttggttttaagtcacgcgaatcaagtcaaaggacaacaaacaccttatgatgctaggatggctggcgcatttaacaaaattaatgggaaatttagcaactgggatgagtatcaaaagtgggtagaaagtcgtccgttcagtcgtaaaaaagtaaataatttattggaaacacggaatatttttgatagtgaacaagttcaaaagtttctgtcacgaaatcttaacgatacgcgttacgctagtcgtttggttttaaatacgttgcaaagcttttttgaaaatcaagacacaattgttcgagtagtaaatggtagtttcactcataccttacgtaaaaaatggggtgctgacttagataaaactcgtgaaacccatcatcatcatgccgttgatgcgacactttgtgcagtgacaccatttgtgaaagtcagtcgttatcactatgcggtcaacgaagaaaccggtgaaaagttcatgcgtgaaattgatgttgaaactggtgaaattctcgatgaaattccgtatagagaatacaaaaaagcaaaacattatgaacgaaaaacgtaccaagtcaaatggtcaaattttcgggaacaattaaaaccaataacgattcatccaaaaattaaattttctcatcaagtggatcgcaaagctaatcgcaaactcagtgatgcaaccatttactctgttcgtgaaaaaacagaagtcaaaacactaaaaagtggtaaagaaaaaataacgacagatgaatataccatcggaaaaattaaagatatttacacggtagatggttgggaagcatttaagaaaaaacaagacaaactattaatgaaagagtttgatgaaaaaacgtacgaattattagtgaccattgcagcaacaacgccagattttcaagaagtggaagaaaaaaatggcaaggtgaaacgtgtgaagcgctcaccatttgctgtttactgcgaagaaaatgggattccagcgattcgaaaatatgcgaaaaaaaataacggtccagttatccgtagcttaaaatattacgatggcaaattaaacaaacatatcaatattaccaaagacgaaaaaggtcgaccagttgaacaaaccaaaaatggtcgcaaagttactttacaaagtctaaaaccctatcgttacgatatttatcaagatttagaaacgaaggcttactatacagtgcaactatattattctgacttgcgttttgtggaaggtgagtatggaattacagaaaaagaatacatgaagaaagtcgctgagcagacaaagggacaagtcgtacgtttctgcttctctttacaaaaaaatgatggtttggaaatcgaatggaaagatagccaacgctatgatgtgcgtttttataatttccaaagtgctaatagcattaactttaaagggttagaacaagaaatgataccagcagaaaatcaatttaagcaaaaaccttataataatggagccatcaatttaaatattgctaaatatggcaaagaaggcaaaaagttacgaaaattcaatacagatattttaggaaagaaacatcatctatcttatgaaaaagaaccaaagaatattattaaaagcggcggaagcaaaagacctgccgctacaaagaaggccggccaggccaagaaaaagaagtaagctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaagcttggtaggaacccctagtgatggagttggccactccctctctgcgcgctcgctcgctcactgaggccgggcgaccaaaggtcgcccgacgcccgggctttgcccgggcggcctcagtgagcgagcgagcgcgcagaggcatttaattaagcaagctgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtcc

SEQ ID NO: 34 - DNA, 7067 bp, synthetic construct
  source 1..7067: mol_type = other DNA
gtgcacgatatcgggtccccaattgctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgtcgacaatgcacgcgttcgaagtactcatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccggcctccaggatgaagtgcacagttattgtactcaaaggaatctacaataataaggcatcttgccgaatttaccgccctacatatgtagggcggtttttttttgtctagacaagttggagctcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagcggccgcgccaccatggcccctaagaagaaaagaaaggtgatgtactctatcggcctcgatctgggcatctctagcgtcggctggtcggtgatcgacgaagagacaggcaaaatcgttgacctgggcgtgcggctgttttctgctaagaacagcgagaagaatctggagagaagaaccagcagaggcgcaagaagactgatcagaagaaagacgaaccgactaaaagacgccaagaaattactggaggccatcggcttctacgaagataaggcccttaaaaatgtctgcccttaccaactgagagtgaagggcctgaccgagggcctgacaaagggcgaactgtacaaggtcgtgctgcacatcgtgaagaaacggggcatcagctacctggacgaggacgacgccgaagctgccaaagaatctcaggactacaaggagcaggtacgcaaaaatgcccaactgctgacaaagtacacccctggacagatccaactgcagagactgaaggagaacaacagagtgaagacaggaatcaacggccagggccactaccaactgaacgtgttcaaggtgagcgcttacgctgacgagctggccaccatcctgaagacccagcaagccctgtatcctaacgaactgaccgacgactggatcgccctgttcgtgcaacctggaattgccgagaatgccggccttatctacagaaagaggccttactaccacggccctggcaacgaggccaacaacagcccttacggaagatggtccgacttccagaaaacaggccagccagccgctaacatcttcgacaagctaatcggaaaggatttccagggcgagctgcgggccagcggcctgagcctgtctgcccagcaatacaacctgctgaacgacctgaccaatctgaaaattgacggagaggtgtccctgtcccccgagcagaaggaattcatcctgaccgagctgatgaccaaggaattcgccagattcggcgtcaacgacattgccaagctgctgggcgtgaaaaaagaacagttgtctggctggcggctggacaagaagggcaagcccgaaatccacaccctgaagggctacagaaactggagaaagatctttgccgaggccggaatcgacctggccacactgccaacagagaccattgactgcctggccaaggttctgaccctgaacaccgagcgggaaggagtggaaaacaccctggcctttgagctgcctgaactggctgagcctgtgaagagcctggtgttggaccattacaaggaactgagccaaagcatctcaacacaggcttggcacagattcagcctgaagacactgcacctgctgatccccgagctgatcaagagcaccagcgagcagaacaccctgctggaacagttccaactgaaggccggcgtgcgcaagcggtactccgactacaagaagctgcctaccaaggaggtgctggccgagatctacaatcctactgtgaacaaaaccgtcagccaggccttcaaggtgatggacgctctgctggagaagtacggcaaggaccagatccactacatcaccgtggaaatgcctagagatgataacgaggaggaagagcgtaaaagaattaaggaactgcaaacgaagaattctcagagaaagaacgatagccagcaatacttcctgcagaaatccggctggagccaggagaagttccaggccacaatccacaagaatagaagattcctggccaagctgctgtactattttgagcaggacggcgtgtgtgcctacaccgggaaccccatcagccctgagcttctggtgtccgacagcaccgagatcgaccacatcatccctatcagcatcagcctggatgacagcatcaacaacaaagtgctggtgctgtctcacgccaaccaggtgaagggccaacagaccccttacgatgccagaatggccggcgccttcaacaagatcaatggaaagttcagcaactgggacgagtatcaaaagtgggtggaaagcagacctttcagccggaaaaaggtgaacaacctcctggagaccagaaacattttcgacagcgagcaggtgcaaaagtttctgagcagaaacctgaatgatacaagatacgccagcagactggtcctgaataccctgcagagctttttcgagaaccaggataccattgtgcgggtggtcaacggcagcttcacccataccctgagaaagaagtggggcgccgatctggacaaaacccgggagacacaccaccaccacgccgtggacgccacactgtgcgcggtgacacccttcgtgaaggtctctagataccactacgccgtgaacgaggagaccggcgagaaattcatgagagaaatcgatgtggagaccggcgaaatcctggacgaaattccatacagagagtacaagaaggccaagcactacgagagaaaaacctaccaggtgaagtggtccaacttccgcgagcagcttaagcccatcacaatccatcctaagatcaagttcagccaccaggttgacagaaaagccaacagaaagctgagcgacgctaccatctactccgttagagagaagacagaggtgaaaacactgaagagcggcaaggagaagatcacgaccgacgaatacaccatcggcaagatcaaggacatctataccgtagacggctgggaagcctttaaaaagaagcaggacaaactgctcatgaaggaattcgatgaaaagacctacgagctgctggtgaccatcgccgccaccacccctgattttcaggaggtggaagaaaagaacggcaaagtgaagagagtgaagcggagccctttcgccgtgtactgtgaggaaaacggcatacccgccatccggaaatacgccaagaagaacaacggccccgtgatccggagcctgaagtactacgacggcaagctgaataagcacatcaacatcaccaaggatgagaagggcaggcccgtggagcagacaaagaacggccggaaagtgactctgcagagcctgaaaccctaccggtatgatatctaccaggacctcgaaaccaaggcctattatacagtgcaactgtactacagcgacctgcgctttgtggagggcgagtacggaatcacagaaaaagagtacatgaagaaggtggccgaacagaccaaaggacaggtggtgcggttctgcttcagcctgcagaagaacgacggcctcgaaatcgagtggaaggattctcaacggtacgacgtgagattctacaatttccaatctgctaacagcatcaacttcaagggcttagaacaggagatgatccctgctgaaaaccagttcaagcagaagccttacaacaatggcgccatcaacctgaacatcgccaagtatggcaaggagggcaagaaactgagaaagttcaacaccgatatcctgggtaaaaagcaccacctgtcttatgagaaagagccgaagaacatcatcaagagcggcggaagcaaaagacctgccgctacaaagaaggccggccaggccaagaaaaagaagtaagctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaagcttggtaggaacccctagtgatggagttggccactccctctctgcgcgctcgctcgctcactgaggccgggcgaccaaaggtcgcccgacgcccgggctttgcccgggcggcctcagtgagcgagcgagcgcgcagaggcatttaattaagcaagctgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtcc

SEQ ID NO: 35 - DNA, 7344 bp, synthetic construct
  source 1..7344: mol_type = other DNA
gtgcacgatatcgggtccccaattgctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgtcgacaatgcacgcgttcgaagtactcatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccggcctccaggatgaagtgcacagttattgtactcaaaggaatctacaataataaggcatcttgccgaatttaccgccctacatatgtagggcggtttttttttgtctagacaagttggagctcagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatccccgggtaccggtcgccaccatggcccctaagaagaaaagaaaggtgatgtactctatcggcctcgatctgggcatctctagcgtcggctggtcggtgatcgacgaagagacaggcaaaatcgttgacctgggcgtgcggctgttttctgctaagaacagcgagaagaatctggagagaagaaccagcagaggcgcaagaagactgatcagaagaaagacgaaccgactaaaagacgccaagaaattactggaggccatcggcttctacgaagataaggcccttaaaaatgtctgcccttaccaactgagagtgaagggcctgaccgagggcctgacaaagggcgaactgtacaaggtcgtgctgcacatcgtgaagaaacggggcatcagctacctggacgaggacgacgccgaagctgccaaagaatctcaggactacaaggagcaggtacgcaaaaatgcccaactgctgacaaagtacacccctggacagatccaactgcagagactgaaggagaacaacagagtgaagacaggaatcaacggccagggccactaccaactgaacgtgttcaaggtgagcgcttacgctgacgagctggccaccatcctgaagacccagcaagccctgtatcctaacgaactgaccgacgactggatcgccctgttcgtgcaacctggaattgccgagaatgccggccttatctacagaaagaggccttactaccacggccctggcaacgaggccaacaacagcccttacggaagatggtccgacttccagaaaacaggccagccagccgctaacatcttcgacaagctaatcggaaaggatttccagggcgagctgcgggccagcggcctgagcctgtctgcccagcaatacaacctgctgaacgacctgaccaatctgaaaattgacggagaggtgtccctgtcccccgagcagaaggaattcatcctgaccgagctgatgaccaaggaattcgccagattcggcgtcaacgacattgccaagctgctgggcgtgaaaaaagaacagttgtctggctggcggctggacaagaagggcaagcccgaaatccacaccctgaagggctacagaaactggagaaagatctttgccgaggccggaatcgacctggccacactgccaacagagaccattgactgcctggccaaggttctgaccctgaacaccgagcgggaaggagtggaaaacaccctggcctttgagctgcctgaactggctgagcctgtgaagagcctggtgttggaccattacaaggaactgagccaaagcatctcaacacaggcttggcacagattcagcctgaagacactgcacctgctgatccccgagctgatcaagagcaccagcgagcagaacaccctgctggaacagttccaactgaaggccggcgtgcgcaagcggtactccgactacaagaagctgcctaccaaggaggtgctggccgagatctacaatcctactgtgaacaaaaccgtcagccaggccttcaaggtgatggacgctctgctggagaagtacggcaaggaccagatccactacatcaccgtggaaatgcctagagatgataacgaggaggaagagcgtaaaagaattaaggaactgcaaacgaagaattctcagagaaagaacgatagccagcaatacttcctgcagaaatccggctggagccaggagaagttccaggccacaatccacaagaatagaagattcctggccaagctgctgtactattttgagcaggacggcgtgtgtgcctacaccgggaaccccatcagccctgagcttctggtgtccgacagcaccgagatcgaccacatcatccctatcagcatcagcctggatgacagcatcaacaacaaagtgctggtgctgtctcacgccaaccaggtgaagggccaacagaccccttacgatgccagaatggccggcgccttcaacaagatcaatggaaagttcagcaactgggacgagtatcaaaagtgggtggaaagcagacctttcagccggaaaaaggtgaacaacctcctggagaccagaaacattttcgacagcgagcaggtgcaaaagtttctgagcagaaacctgaatgatacaagatacgccagcagactggtcctgaataccctgcagagctttttcgagaaccaggataccattgtgcgggtggtcaacggcagcttcacccataccctgagaaagaagtggggcgccgatctggacaaaacccgggagacacaccaccaccacgccgtggacgccacactgtgcgcggtgacacccttcgtgaaggtctctagataccactacgccgtgaacgaggagaccggcgagaaattcatgagagaaatcgatgtggagaccggcgaaatcctggacgaaattccatacagagagtacaagaaggccaagcactacgagagaaaaacctaccaggtgaagtggtccaacttccgcgagcagcttaagcccatcacaatccatcctaagatcaagttcagccaccaggttgacagaaaagccaacagaaagctgagcgacgctaccatctactccgttagagagaagacagaggtgaaaacactgaagagcggcaaggagaagatcacgaccgacgaatacaccatcggcaagatcaaggacatctataccgtagacggctgggaagcctttaaaaagaagcaggacaaactgctcatgaaggaattcgatgaaaagacctacgagctgctggtgaccatcgccgccaccacccctgattttcaggaggtggaagaaaagaacggcaaagtgaagagagtgaagcggagccctttcgccgtgtactgtgaggaaaacggcatacccgccatccggaaatacgccaagaagaacaacggccccgtgatccggagcctgaagtactacgacggcaagctgaataagcacatcaacatcaccaaggatgagaagggcaggcccgtggagcagacaaagaacggccggaaagtgactctgcagagcctgaaaccctaccggtatgatatctaccaggacctcgaaaccaaggcctattatacagtgcaactgtactacagcgacctgcgctttgtggagggcgagtacggaatcacagaaaaagagtacatgaagaaggtggccgaacagaccaaaggacaggtggtgcggttctgcttcagcctgcagaagaacgacggcctcgaaatcgagtggaaggattctcaacggtacgacgtgagattctacaatttccaatctgctaacagcatcaacttcaagggcttagaacaggagatgatccctgctgaaaaccagttcaagcagaagccttacaacaatggcgccatcaacctgaacatcgccaagtatggcaaggagggcaagaaactgagaaagttcaacaccgatatcctgggtaaaaagcaccacctgtcttatgagaaagagccgaagaacatcatcaagagcggcggaagcaaaagacctgccgctacaaagaaggccggccaggccaagaaaaagaagtaagctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaagcttggtaggaacccctagtgatggagttggccactccctctctgcgcgctcgctcgctcactgaggccgggcgaccaaaggtcgcccgacgcccgggctttgcccgggcggcctcagtgagcgagcgagcgcgcagaggcatttaattaagcaagctgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtcc

SEQ ID NO: 36 - DNA, 7102 bp, synthetic construct
  source 1..7102: mol_type = other DNA
gtgcacgatatcgggtccccaattgctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgtcgacaatgcacgcgttcgaagtactcatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccggcctccaggatgaagtgcacagttattgtactcaaaggaatctacaataataaggcatcttgccgaatttaccgccctacatatgtagggcggtttttttttgtctagacaagttggagctcgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctctttgtcgagccaccatggcccctaagaagaaaagaaaggtgatgtactctatcggcctcgatctgggcatctctagcgtcggctggtcggtgatcgacgaagagacaggcaaaatcgttgacctgggcgtgcggctgttttctgctaagaacagcgagaagaatctggagagaagaaccagcagaggcgcaagaagactgatcagaagaaagacgaaccgactaaaagacgccaagaaattactggaggccatcggcttctacgaagataaggcccttaaaaatgtctgcccttaccaactgagagtgaagggcctgaccgagggcctgacaaagggcgaactgtacaaggtcgtgctgcacatcgtgaagaaacggggcatcagctacctggacgaggacgacgccgaagctgccaaagaatctcaggactacaaggagcaggtacgcaaaaatgcccaactgctgacaaagtacacccctggacagatccaactgcagagactgaaggagaacaacagagtgaagacaggaatcaacggccagggccactaccaactgaacgtgttcaaggtgagcgcttacgctgacgagctggccaccatcctgaagacccagcaagccctgtatcctaacgaactgaccgacgactggatcgccctgttcgtgcaacctggaattgccgagaatgccggccttatctacagaaagaggccttactaccacggccctggcaacgaggccaacaacagcccttacggaagatggtccgacttccagaaaacaggccagccagccgctaacatcttcgacaagctaatcggaaaggatttccagggcgagctgcgggccagcggcctgagcctgtctgcccagcaatacaacctgctgaacgacctgaccaatctgaaaattgacggagaggtgtccctgtcccccgagcagaaggaattcatcctgaccgagctgatgaccaaggaattcgccagattcggcgtcaacgacattgccaagctgctgggcgtgaaaaaagaacagttgtctggctggcggctggacaagaagggcaagcccgaaatccacaccctgaagggctacagaaactggagaaagatctttgccgaggccggaatcgacctggccacactgccaacagagaccattgactgcctggccaaggttctgaccctgaacaccgagcgggaaggagtggaaaacaccctggcctttgagctgcctgaactggctgagcctgtgaagagcctggtgttggaccattacaaggaactgagccaaagcatctcaacacaggcttggcacagattcagcctgaagacactgcacctgctgatccccgagctgatcaagagcaccagcgagcagaacaccctgctggaacagttccaactgaaggccggcgtgcgcaagcggtactccgactacaagaagctgcctaccaaggaggtgctggccgagatctacaatcctactgtgaacaaaaccgtcagccaggccttcaaggtgatggacgctctgctggagaagtacggcaaggaccagatccactacatcaccgtggaaatgcctagagatgataacgaggaggaagagcgtaaaagaattaaggaactgcaaacgaagaattctcagagaaagaacgatagccagcaatacttcctgcagaaatccggctggagccaggagaagttccaggccacaatccacaagaatagaagattcctggccaagctgctgtactattttgagcaggacggcgtgtgtgcctacaccgggaaccccatcagccctgagcttctggtgtccgacagcaccgagatcgaccacatcatccctatcagcatcagcctggatgacagcatcaacaacaaagtgctggtgctgtctcacgccaaccaggtgaagggccaacagaccccttacgatgccagaatggccggcgccttcaacaagatcaatggaaagttcagcaactgggacgagtatcaaaagtgggtggaaagcagacctttcagccggaaaaaggtgaacaacctcctggagaccagaaacattttcgacagcgagcaggtgcaaaagtttctgagcagaaacctgaatgatacaagatacgccagcagactggtcctgaataccctgcagagctttttcgagaaccaggataccattgtgcgggtggtcaacggcagcttcacccataccctgagaaagaagtggggcgccgatctggacaaaacccgggagacacaccaccaccacgccgtggacgccacactgtgcgcggtgacacccttcgtgaaggtctctagataccactacgccgtgaacgaggagaccggcgagaaattcatgagagaaatcgatgtggagaccggcgaaatcctggacgaaattccatacagagagtacaagaaggccaagcactacgagagaaaaacctaccaggtgaagtggtccaacttccgcgagcagcttaagcccatcacaatccatcctaagatcaagttcagccaccaggttgacagaaaagccaacagaaagctgagcgacgctaccatctactccgttagagagaagacagaggtgaaaacactgaagagcggcaaggagaagatcacgaccgacgaatacaccatcggcaagatcaaggacatctataccgtagacggctgggaagcctttaaaaagaagcaggacaaactgctcatgaaggaattcgatgaaaagacctacgagctgctggtgaccatcgccgccaccacccctgattttcaggaggtggaagaaaagaacggcaaagtgaagagagtgaagcggagccctttcgccgtgtactgtgaggaaaacggcatacccgccatccggaaatacgccaagaagaacaacggccccgtgatccggagcctgaagtactacgacggcaagctgaataagcacatcaacatcaccaaggatgagaagggcaggcccgtggagcagacaaagaacggccggaaagtgactctgcagagcctgaaaccctaccggtatgatatctaccaggacctcgaaaccaaggcctattatacagtgcaactgtactacagcgacctgcgctttgtggagggcgagtacggaatcacagaaaaagagtacatgaagaaggtggccgaacagaccaaaggacaggtggtgcggttctgcttcagcctgcagaagaacgacggcctcgaaatcgagtggaaggattctcaacggtacgacgtgagattctacaatttccaatctgctaacagcatcaacttcaagggcttagaacaggagatgatccctgctgaaaaccagttcaagcagaagccttacaacaatggcgccatcaacctgaacatcgccaagtatggcaaggagggcaagaaactgagaaagttcaacaccgatatcctgggtaaaaagcaccacctgtcttatgagaaagagccgaagaacatcatcaagagcggcggaagcaaaagacctgccgctacaaagaaggccggccaggccaagaaaaagaagtaagctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaagcttggtaggaacccctagtgatggagttggccactccctctctgcgcgctcgctcgctcactgaggccgggcgaccaaaggtcgcccgacgcccgggctttgcccgggcggcctcagtgagcgagcgagcgcgcagaggcatttaattaagcaagctgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtcc

SEQ ID NO: 37 - DNA, 7144 bp, synthetic construct
  source 1..7144: mol_type = other DNA
gtgcacgatatcgggtccccaattgctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgtcgacaatgcacgcgttcgaagtactcatgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccggcctccaggatgaagtgcacagttattgtactcaaaggaatctacaataataaggcatcttgccgaatttaccgccctacatatgtagggcggtttttttttgtctagacaagttggagctcgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccgccaccatggcccctaagaagaaaagaaaggtgatgtactctatcggcctcgatctgggcatctctagcgtcggctggtcggtgatcgacgaagagacaggcaaaatcgttgacctgggcgtgcggctgttttctgctaagaacagcgagaagaatctggagagaagaaccagcagaggcgcaagaagactgatcagaagaaagacgaaccgactaaaagacgccaagaaattactggaggccatcggcttctacgaagataaggcccttaaaaatgtctgcccttaccaactgagagtgaagggcctgaccgagggcctgacaaagggcgaactgtacaaggtcgtgctgcacatcgtgaagaaacggggcatcagctacctggacgaggacgacgccgaagctgccaaagaatctcaggactacaaggagcaggtacgcaaaaatgcccaactgctgacaaagtacacccctggacagatccaactgcagagactgaaggagaacaacagagtgaagacaggaatcaacggccagggccactaccaactgaacgtgttcaaggtgagcgcttacgctgacgagctggccaccatcctgaagacccagcaagccctgtatcctaacgaactgaccgacgactggatcgccctgttcgtgcaacctggaattgccgagaatgccggccttatctacagaaagaggccttactaccacggccctggcaacgaggccaacaacagcccttacggaagatggtccgacttccagaaaacaggccagccagccgctaacatcttcgacaagctaatcggaaaggatttccagggcgagctgcgggccagcggcctgagcctgtctgcccagcaatacaacctgctgaacgacctgaccaatctgaaaattgacggagaggtgtccctgtcccccgagcagaaggaattcatcctgaccgagctgatgaccaaggaattcgccagattcggcgtcaacgacattgccaagctgctgggcgtgaaaaaagaacagttgtctggctggcggctggacaagaagggcaagcccgaaatccacaccctgaagggctacagaaactggagaaagatctttgccgaggccggaatcgacctggccacactgccaacagagaccattgactgcctggccaaggttctgaccctgaacaccgagcgggaaggagtggaaaacaccctggcctttgagctgcctgaactggctgagcctgtgaagagcctggtgttggaccattacaaggaactgagccaaagcatctcaacacaggcttggcacagattcagcctgaagacactgcacctgctgatccccgagctgatcaagagcaccagcgagcagaacaccctgctggaacagttccaactgaaggccggcgtgcgcaagcggtactccgactacaagaagctgcctaccaaggaggtgctggccgagatctacaatcctactgtgaacaaaaccgtcagccaggccttcaaggtgatggacgctctgctggagaagtacggcaaggaccagatccactacatcaccgtggaaatgcctagagatgataacgaggaggaagagcgtaaaagaattaaggaactgcaaacgaagaattctcagagaaagaacgatagccagcaatacttcctgcagaaatccggctggagccaggagaagttccaggccacaatccacaagaatagaagattcctggccaagctgctgtactattttgagcaggacggcgtgtgtgcctacaccgggaaccccatcagccctgagcttctggtgtccgacagcaccgagatcgaccacatcatccctatcagcatcagcctggatgacagcatcaacaacaaagtgctggtgctgtctcacgccaaccaggtgaagggccaacagaccccttacgatgccagaatggccggcgccttcaacaagatcaatggaaagttcagcaactgggacgagtatcaaaagtgggtggaaagcagacctttcagccggaaaaaggtgaacaacctcctggagaccagaaacattttcgacagcgagcaggtgcaaaagtttctgagcagaaacctgaatgatacaagatacgccagcagactggtcctgaataccctgcagagctttttcgagaaccaggataccattgtgcgggtggtcaacggcagcttcacccataccctgagaaagaagtggggcgccgatctggacaaaacccgggagacacaccaccaccacgccgtggacgccacactgtgcgcggtgacacccttcgtgaaggtctctagataccactacgccgtgaacgaggagaccggcgagaaattcatgagagaaatcgatgtggagaccggcgaaatcctggacgaaattccatacagagagtacaagaaggccaagcactacgagagaaaaacctaccaggtgaagtggtccaacttccgcgagcagcttaagcccatcacaatccatcctaagatcaagttcagccaccaggttgacagaaaagccaacagaaagctgagcgacgctaccatctactccgttagagagaagacagaggtgaaaacactgaagagcggcaaggagaagatcacgaccgacgaatacaccatcggcaagatcaaggacatctataccgtagacggctgggaagcctttaaaaagaagcaggacaaactgctcatgaaggaattcgatgaaaagacctacgagctgctggtgaccatcgccgccaccacccctgattttcaggaggtggaagaaaagaacggcaaagtgaagagagtgaagcggagccctttcgccgtgtactgtgaggaaaacggcatacccgccatccggaaatacgccaagaagaacaacggccccgtgatccggagcctgaagtactacgacggcaagctgaataagcacatcaacatcaccaaggatgagaagggcaggcccgtggagcagacaaagaacggccggaaagtgactctgcagagcctgaaaccctaccggtatgatatctaccaggacctcgaaaccaaggcctattatacagtgcaactgtactacagcgacctgcgctttgtggagggcgagtacggaatcacagaaaaagagtacatgaagaaggtggccgaacagaccaaaggacaggtggtgcggttctgcttcagcctgcagaagaacgacggcctcgaaatcgagtggaaggattctcaacggtacgacgtgagattctacaatttccaatctgctaacagcatcaacttcaagggcttagaacaggagatgatccctgctgaaaaccagttcaagcagaagccttacaacaatggcgccatcaacctgaacatcgccaagtatggcaaggagggcaagaaactgagaaagttcaacaccgatatcctgggtaaaaagcaccacctgtcttatgagaaagagccgaagaacatcatcaagagcggcggaagcaaaagacctgccgctacaaagaaggccggccaggccaagaaaaagaagtaagctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaagcttggtaggaacccctagtgatggagttggccactccctctctgcgcgctcgctcgctcactgaggccgggcgaccaaaggtcgcccgacgcccgggctttgcccgggcggcctcagtgagcgagcgagcgcgcagaggcatttaattaagcaagctgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtcc

SEQ ID NO: 38 - DNA, 7079 bp, synthetic construct
  source 1..7079: mol_type = other DNA
gtgcacgatatcgggtccccaattgctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgtcgacaatgcacgcgttcgaagtactcagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccggcctccaggatgaagtgcacagttattgtactcaaaggaatctacaataataaggcatcttgccgaatttaccgccctacatatgtagggcggtttttttttgtctagacaagttggagctcgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctctttgtcgagccaccatggcccctaagaagaaaagaaaggtgatgtactctatcggcctcgatctgggcatctctagcgtcggctggtcggtgatcgacgaagagacaggcaaaatcgttgacctgggcgtgcggctgttttctgctaagaacagcgagaagaatctggagagaagaaccagcagaggcgcaagaagactgatcagaagaaagacgaaccgactaaaagacgccaagaaattactggaggccatcggcttctacgaagataaggcccttaaaaatgtctgcccttaccaactgagagtgaagggcctgaccgagggcctgacaaagggcgaactgtacaaggtcgtgctgcacatcgtgaagaaacggggcatcagctacctggacgaggacgacgccgaagctgccaaagaatctcaggactacaaggagcaggtacgcaaaaatgcccaactgctgacaaagtacacccctggacagatccaactgcagagactgaaggagaacaacagagtgaagacaggaatcaacggccagggccactaccaactgaacgtgttcaaggtgagcgcttacgctgacgagctggccaccatcctgaagacccagcaagccctgtatcctaacgaactgaccgacgactggatcgccctgttcgtgcaacctggaattgccgagaatgccggccttatctacagaaagaggccttactaccacggccctggcaacgaggccaacaacagcccttacggaagatggtccgacttccagaaaacaggccagccagccgctaacatcttcgacaagctaatcggaaaggatttccagggcgagctgcgggccagcggcctgagcctgtctgcccagcaatacaacctgctgaacgacctgaccaatctgaaaattgacggagaggtgtccctgtcccccgagcagaaggaattcatcctgaccgagctgatgaccaaggaattcgccagattcggcgtcaacgacattgccaagctgctgggcgtgaaaaaagaacagttgtctggctggcggctggacaagaagggcaagcccgaaatccacaccctgaagggctacagaaactggagaaagatctttgccgaggccggaatcgacctggccacactgccaacagagaccattgactgcctggccaaggttctgaccctgaacaccgagcgggaaggagtggaaaacaccctggcctttgagctgcctgaactggctgagcctgtgaagagcctggtgttggaccattacaaggaactgagccaaagcatctcaacacaggcttggcacagattcagcctgaagacactgcacctgctgatccccgagctgatcaagagcaccagcgagcagaacaccctgctggaacagttccaactgaaggccggcgtgcgcaagcggtactccgactacaagaagctgcctaccaaggaggtgctggccgagatctacaatcctactgtgaacaaaaccgtcagccaggccttcaaggtgatggacgctctgctggagaagtacggcaaggaccagatccactacatcaccgtggaaatgcctagagatgataacgaggaggaagagcgtaaaagaattaaggaactgcaaacgaagaattctcagagaaagaacgatagccagcaatacttcctgcagaaatccggctggagccaggagaagttccaggccacaatccacaagaatagaagattcctggccaagctgctgtactattttgagcaggacggcgtgtgtgcctacaccgggaaccccatcagccctgagcttctggtgtccgacagcaccgagatcgaccacatcatccctatcagcatcagcctggatgacagcatcaacaacaaagtgctggtgctgtctcacgccaaccaggtgaagggccaacagaccccttacgatgccagaatggccggcgccttcaacaagatcaatggaaagttcagcaactgggacgagtatcaaaagtgggtggaaagcagacctttcagccggaaaaaggtgaacaacctcctggagaccagaaacattttcgacagcgagcaggtgcaaaagtttctgagcagaaacctgaatgatacaagatacgccagcagactggtcctgaataccctgcagagctttttcgagaaccaggataccattgtgcgggtggtcaacggcagcttcacccataccctgagaaagaagtggggcgccgatctggacaaaacccgggagacacaccaccaccacgccgtggacgccacactgtgcgcggtgacacccttcgtgaaggtctctagataccactacgccgtgaacgaggagaccggcgagaaattcatgagagaaatcgatgtggagaccggcgaaatcctggacgaaattccatacagagagtacaagaaggccaagcactacgagagaaaaacctaccaggtgaagtggtccaacttccgcgagcagcttaagcccatcacaatccatcctaagatcaagttcagccaccaggttgacagaaaagccaacagaaagctgagcgacgctaccatctactccgttagagagaagacagaggtgaaaacactgaagagcggcaaggagaagatcacgaccgacgaatacaccatcggcaagatcaaggacatctataccgtagacggctgggaagcctttaaaaagaagcaggacaaactgctcatgaaggaattcgatgaaaagacctacgagctgctggtgaccatcgccgccaccacccctgattttcaggaggtggaagaaaagaacggcaaagtgaagagagtgaagcggagccctttcgccgtgtactgtgaggaaaacggcatacccgccatccggaaatacgccaagaagaacaacggccccgtgatccggagcctgaagtactacgacggcaagctgaataagcacatcaacatcaccaaggatgagaagggcaggcccgtggagcagacaaagaacggccggaaagtgactctgcagagcctgaaaccctaccggtatgatatctaccaggacctcgaaaccaaggcctattatacagtgcaactgtactacagcgacctgcgctttgtggagggcgagtacggaatcacagaaaaagagtacatgaagaaggtggccgaacagaccaaaggacaggtggtgcggttctgcttcagcctgcagaagaacgacggcctcgaaatcgagtggaaggattctcaacggtacgacgtgagattctacaatttccaatctgctaacagcatcaacttcaagggcttagaacaggagatgatccctgctgaaaaccagttcaagcagaagccttacaacaatggcgccatcaacctgaacatcgccaagtatggcaaggagggcaagaaactgagaaagttcaacaccgatatcctgggtaaaaagcaccacctgtcttatgagaaagagccgaagaacatcatcaagagcggcggaagcaaaagacctgccgctacaaagaaggccggccaggccaagaaaaagaagtaagctagcttgactgactgagatacagcgtaccttcagctcacagacatgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttttaaagcaagtaaaagcttggtaggaacccctagtgatggagttggccactccctctctgcgcgctcgctcgctcactgaggccgggcgaccaaaggtcgcccgacgcccgggctttgcccgggcggcctcagtgagcgagcgagcgcgcagaggcatttaattaagcaagctgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtcc

SEQ ID NO: 39 - DNA, 7121 bp, synthetic construct
  source 1..7121: mol_type = other DNA
gtgcacgatatcgggtccccaattgctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgtcgacaatgcacgcgttcgaagtactcagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccggcctccaggatgaagtgcacagttattgtactcaaaggaatctacaataataaggcatcttgccgaatttaccgccctacatatgtagggcggtttttttttgtctagacaagttggagctcgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccgccaccatggcccctaagaagaaaagaaaggtgatgtactctatcggcctcgatctgggcatctctagcgtcggctggtcggtgatcgacgaagagacaggcaaaatcgttgacctgggcgtgcggctgttttctgctaagaacagcgagaagaatctggagagaagaaccagcagaggcgcaagaagactgatcagaagaaagacgaaccgactaaaagacgccaagaaattactggaggccatcggcttctacgaagataaggcccttaaaaatgtctgcccttaccaactgagagtgaagggcctgaccgagggcctgacaaagggcgaactgtacaaggtcgtgctgcacatcgtgaagaaacggggcatcagctacctggacgaggacgacgccgaagctgccaaagaatctcaggactacaaggagcaggtacgcaaaaatgcccaactgctgacaaagtacacccctggacagatccaactgcagagactgaaggagaacaacagagtgaagacaggaatcaacggccagggccactaccaactgaacgtgttcaaggtgagcgcttacgctgacgagctggccaccatcctgaagacccagcaagccctgtatcctaacgaactgaccgacgactggatcgccctgttcgtgcaacctggaattgccgagaatgccggccttatctacagaaagaggccttactaccacggccctggcaacgaggccaacaacagcccttacggaagatggtccgacttccagaaaacaggccagccagccgctaacatcttcgacaagctaatcggaaaggatttccagggcgagctgcgggccagcggcctgagcctgtctgcccagcaatacaacctgctgaacgacctgaccaatctgaaaattgacggagaggtgtccctgtcccccgagcagaaggaattcatcctgaccgagctgatgaccaaggaattcgccagattcggcgtcaacgacattgccaagctgctgggcgtgaaaaaagaacagttgtctggctggcggctggacaagaagggcaagcccgaaatccacaccctgaagggctacagaaactggagaaagatctttgccgaggccggaatcgacctggccacactgccaacagagaccattgactgcctggccaaggttctgaccctgaacaccgagcgggaaggagtggaaaacaccctggcctttgagctgcctgaactggctgagcctgtgaagagcctggtgttggaccattacaaggaactgagccaaagcatctcaacacaggcttggcacagattcagcctgaagacactgcacctgctgatccccgagctgatcaagagcaccagcgagcagaacaccctgctggaacagttccaactgaaggccggcgtgcgcaagcggtactccgactacaagaagctgcctaccaaggaggtgctggccgagatctacaatcctactgtgaacaaaaccgtcagccaggccttcaaggtgatggacgctctgctggagaagtacggcaaggaccagatccactacatcaccgtggaaatgcctagagatgataacgaggaggaagagcgtaaaagaattaaggaactgcaaacgaagaattctcagagaaagaacgatagccagcaatacttcctgcagaaatccggctggagccaggagaagttccaggccacaatccacaagaatagaagattcctggccaagctgctgtactattttgagcaggacggcgtgtgtgcctacaccgggaaccccatcagccctgagcttctggtgtccgacagcaccgagatcgaccacatcatccctatcagcatcagcctggatgacagcatcaacaacaaagtgctggtgctgtctcacgccaaccaggtgaagggccaacagaccccttacgatgccagaatggccggcgccttcaacaagatcaatggaaagttcagcaactgggacgagtatcaaaagtgggtggaaagcagacctttcagccggaaaaaggtgaacaacctcctggagaccagaaacattttcgacagcgagcaggtgcaaaagtttctgagcagaaacctgaatgatacaagatacgccagcagactggtcctgaataccctgcagagctttttcgagaaccaggataccattgtgcgggtggtcaacggcagcttcacccataccctgagaaagaagtggggcgccgatctggacaaaacccgggagacacaccaccaccacgccgtggacgccacactgtgcgcggtgacacccttcgtgaaggtctctagataccactacgccgtgaacgaggagaccggcgagaaattcatgagagaaatcgatgtggagaccggcgaaatcctggacgaaattccatacagagagtacaagaaggccaagcactacgagagaaaaacctaccaggtgaagtggtccaacttccgcgagcagcttaagcccatcacaatccatcctaagatcaagttcagccaccaggttgacagaaaagccaacagaaagctgagcgacgctaccatctactccgttagagagaagacagaggtgaaaacactgaagagcggcaaggagaagatcacgaccgacgaatacaccatcggcaagatcaaggacatctataccgtagacggctgggaagcctttaaaaagaagcaggacaaactgctcatgaaggaattcgatgaaaagacctacgagctgctggtgaccatcgccgccaccacccctgattttcaggaggtggaagaaaagaacggcaaagtgaagagagtgaagcggagccctttcgccgtgtactgtgaggaaaacggcatacccgccatccggaaatacgccaagaagaacaacggccccgtgatccggagcctgaagtactacgacggcaagctgaataagcacatcaacatcaccaaggatgagaagggcaggcccgtggagcagacaaagaacggccggaaagtgactctgcagagcctgaaaccctaccggtatgatatctaccaggacctcgaaaccaaggcctattatacagtgcaactgtactacagcgacctgcgctttgtggagggcgagtacggaatcacagaaaaagagtacatgaagaaggtggccgaacagaccaaaggacaggtggtgcggttctgcttcagcctgcagaagaacgacggcctcgaaatcgagtggaaggattctcaacggtacgacgtgagattctacaatttccaatctgctaacagcatcaacttcaagggcttagaacaggagatgatccctgctgaaaaccagttcaagcagaagccttacaacaatggcgccatcaacctgaacatcgccaagtatggcaaggagggcaagaaactgagaaagttcaacaccgatatcctgggtaaaaagcaccacctgtcttatgagaaagagccgaagaacatcatcaagagcggcggaagcaaaagacctgccgctacaaagaaggccggccaggccaagaaaaagaagtaagctagcttgactgactgagatacagcgtaccttcagctcacagacatgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttttaaagcaagtaaaagcttggtaggaacccctagtgatggagttggccactccctctctgcgcgctcgctcgctcactgaggccgggcgaccaaaggtcgcccgacgcccgggctttgcccgggcggcctcagtgagcgagcgagcgcgcagaggcatttaattaagcaagctgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtcc

SEQ ID NO: 40 - DNA, 25 bp, Homo sapiens
  source 1..25: mol_type = genomic DNA
cccagcctccaggatgcagtgttca

SEQ ID NO: 41 - DNA, 20 bp, synthetic construct
  source 1..20: mol_type = other DNA
tgatgcaagctaccaccagg

SEQ ID NO: 42 - DNA, 20 bp, synthetic construct
  source 1..20: mol_type = other DNA
ttcaggaagctgagccactg

SEQ ID NO: 43 - DNA, 25 bp, Homo sapiens
  source 1..25: mol_type = genomic DNA
tctgtcatccaggttgaagtgcaca

SEQ ID NO: 44 - DNA, 20 bp, synthetic construct
  source 1..20: mol_type = other DNA
catggcaagtccccgtctct

SEQ ID NO: 45 - DNA, 23 bp, synthetic construct
  source 1..23: mol_type = other DNA
tcactctcacacccaagattcca

SEQ ID NO: 46 - DNA, 24 bp, Homo sapiens
  source 1..24: mol_type = genomic DNA
cccgtcccccaggctgaagtgcaa

SEQ ID NO: 47 - DNA, 20 bp, synthetic construct
  source 1..20: mol_type = other DNA
gtcctagctactggggcagc

SEQ ID NO: 48 - DNA, 21 bp, synthetic construct
  source 1..21: mol_type = other DNA
gtcccaggggtacaccaatgt

SEQ ID NO: 49 - DNA, 24 bp, Homo sapiens
  source 1..24: mol_type = genomic DNA
tctgtcctccaggctgaagtgcaa

SEQ ID NO: 50 - DNA, 20 bp, synthetic construct
  source 1..20: mol_type = other DNA
ggacaggagctggtcgttga

SEQ ID NO: 51 - DNA, 20 bp, synthetic construct
  source 1..20: mol_type = other DNA
atgtagctgggcatggtggt

SEQ ID NO: 52 - DNA, 24 bp, Homo sapiens
  source 1..24: mol_type = genomic DNA
tctgtcctccaggctgaagtgcaa

SEQ ID NO: 53 - DNA, 20 bp, synthetic construct
  source 1..20: mol_type = other DNA
ttgcttaccttccccactgg

SEQ ID NO: 54 - DNA, 16 bp, synthetic construct
  source 1..16: mol_type = other DNA
agctaggcgtggtggc

SEQ ID NO: 55 - DNA, 24 bp, Homo sapiens
  source 1..24: mol_type = genomic DNA
tccgtcgtccaggatggagtgcaa

SEQ ID NO: 56 - DNA, 20 bp, synthetic construct
  source 1..20: mol_type = other DNA
ccacgcatggcctccatttc

SEQ ID NO: 57 - DNA, 20 bp, synthetic construct
  source 1..20: mol_type = other DNA
atggaaattagggccgggcg

SEQ ID NO: 58 - DNA, 26 bp, Homo sapiens
  source 1..26: mol_type = genomic DNA
acaggcccccggtatgaagtgcagca

SEQ ID NO: 59 - DNA, 20 bp, synthetic construct
  source 1..20: mol_type = other DNA
ccgaaatcaggaaggccctt

SEQ ID NO: 60 - DNA, 20 bp, synthetic construct
  source 1..20: mol_type = other DNA
cttctatggtggcaccggtt

SEQ ID NO: 61 - DNA, 24 bp, Homo sapiens
  source 1..24: mol_type = genomic DNA
tctgtcctccaggatgaagtgcaa

SEQ ID NO: 62 - DNA, 20 bp, synthetic construct
  source 1..20: mol_type = other DNA
cgtgctctgttccatccctt

SEQ ID NO: 63 - DNA, 20 bp, synthetic construct
  source 1..20: mol_type = other DNA
agtcccagctactcaggagg

SEQ ID NO: 64 - DNA, 25 bp, Homo sapiens
  source 1..25: mol_type = genomic DNA
actctgcctccaggctgaagtgcag

SEQ ID NO: 65 - DNA, 18 bp, synthetic construct
  source 1..18: mol_type = other DNA
tagctgggcgtggttgca

SEQ ID NO: 66 - DNA, 20 bp, synthetic construct
  source 1..20: mol_type = other DNA
cactgtgctcccaacttgcc

SEQ ID NO: 67 - DNA, 20 bp, synthetic construct
  source 1..20: mol_type = other DNA
cattacaggcgctgctcata

SEQ ID NO: 68 - DNA, 20 bp, synthetic construct
  source 1..20: mol_type = other DNA
ttgcccttcttgtcaattcc

SEQ ID NO: 69 - DNA, 23 bp, synthetic construct
  source 1..23: mol_type = other DNA
tcccttccgcatgatggtctgct

SEQ ID NO: 70 - DNA, 53 bp, synthetic construct
  source 1..53: mol_type = other DNA
tcgtcggcagcgtcagatgtgtataagagacagaaacgaaggtacacgagtgg

SEQ ID NO: 71 - DNA, 54 bp, synthetic construct
  source 1..54: mol_type = other DNA
gtctcgtgggctcggagatgtgtataagagacaggcaaacaggcagcacaaaat

SEQ ID NO: 72 - DNA, 20 bp, synthetic construct
  source 1..20: mol_type = other DNA
cattacaggcgctgctcata

SEQ ID NO: 73 - DNA, 20 bp, synthetic construct
  source 1..20: mol_type = other DNA
ttgcccttcttgtcaattcc

SEQ ID NO: 74 - DNA, 23 bp, synthetic construct
  source 1..23: mol_type = other DNA
tcccttccgcatgatggtctgct

SEQ ID NO: 75 - DNA, 25 bp, Homo sapiens
  source 1..25: mol_type = genomic DNA
accggcctccaggatgaagtgcaca

SEQ ID NO: 76 - DNA, 25 bp, Homo sapiens
  source 1..25: mol_type = genomic DNA
accggcctccaggatgaagtgcaca

SEQ ID NO: 77 - DNA, 25 bp, Homo sapiens
  source 1..25: mol_type = genomic DNA
gagtttgtgacccacgcctgctccc

SEQ ID NO: 78 - DNA, 22 bp, Homo sapiens
  source 1..22: mol_type = genomic DNA
tttgtgacccacgcctgctccc

SEQ ID NO: 79 - DNA, 25 bp, Homo sapiens
  source 1..25: mol_type = genomic DNA
cggcctccaggatgaagtgcacaca

SEQ ID NO: 80 - RNA, 25 nt, synthetic construct
  source 1..25: mol_type = other RNA
accggcctccaggatgaagtgcaca

SEQ ID NO: 81 - RNA, 25 nt, synthetic construct
  source 1..25: mol_type = other RNA
accggcctccaggatgaagtgcaca

SEQ ID NO: 82 - RNA, 25 nt, synthetic construct
  source 1..25: mol_type = other RNA
gagtttgtgacccacgcctgctccc

SEQ ID NO: 83 - RNA, 22 nt, synthetic construct
  source 1..22: mol_type = other RNA
tttgtgacccacgcctgctccc

SEQ ID NO: 84 - RNA, 25 nt, synthetic construct
  source 1..25: mol_type = other RNA
cggcctccaggatgaagtgcacaca

SEQ ID NO: 85 - protein, 3142 aa, Homo sapiens
MATLEKLMKAFESLKSFQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 86 - protein, 7 aa, synthetic construct
PKKKRKV

SEQ ID NO: 87 - protein, 16 aa, synthetic construct
KRPAATKKAGQAKKKK

SEQ ID NO: 88 - DNA, 3450 bp, synthetic construct
  source 1..3450: mol_type = other DNA
atgtactctatcggcctcgatctgggcatctctagcgtcggctggtcggtgatcgacgaagagacaggcaaaatcgttgacctgggcgtgcggctgttttctgctaagaacagcgagaagaatctggagagaagaaccagcagaggcgcaagaagactgatcagaagaaagacgaaccgactaaaagacgccaagaaattactggaggccatcggcttctacgaagataaggcccttaaaaatgtctgcccttaccaactgagagtgaagggcctgaccgagggcctgacaaagggcgaactgtacaaggtcgtgctgcacatcgtgaagaaacggggcatcagctacctggacgaggacgacgccgaagctgccaaagaatctcaggactacaaggagcaggtacgcaaaaatgcccaactgctgacaaagtacacccctggacagatccaactgcagagactgaaggagaacaacagagtgaagacaggaatcaacggccagggccactaccaactgaacgtgttcaaggtgagcgcttacgctgacgagctggccaccatcctgaagacccagcaagccctgtatcctaacgaactgaccgacgactggatcgccctgttcgtgcaacctggaattgccgagaatgccggccttatctacagaaagaggccttactaccacggccctggcaacgaggccaacaacagcccttacggaagatggtccgacttccagaaaacaggccagccagccgctaacatcttcgacaagctaatcggaaaggatttccagggcgagctgcgggccagcggcctgagcctgtctgcccagcaatacaacctgctgaacgacctgaccaatctgaaaattgacggagaggtgtccctgtcccccgagcagaaggaattcatcctgaccgagctgatgaccaaggaattcgccagattcggcgtcaacgacattgccaagctgctgggcgtgaaaaaagaacagttgtctggctggcggctggacaagaagggcaagcccgaaatccacaccctgaagggctacagaaactggagaaagatctttgccgaggccggaatcgacctggccacactgccaacagagaccattgactgcctggccaaggttctgaccctgaacaccgagcgggaaggagtggaaaacaccctggcctttgagctgcctgaactggctgagcctgtgaagagcctggtgttggaccattacaaggaactgagccaaagcatctcaacacaggcttggcacagattcagcctgaagacactgcacctgctgatccccgagctgatcaagagcaccagcgagcagaacaccctgctggaacagttccaactgaaggccggcgtgcgcaagcggtactccgactacaagaagctgcctaccaaggaggtgctggccgagatctacaatcctactgtgaacaaaaccgtcagccaggccttcaaggtgatggacgctctgctggagaagtacggcaaggaccagatccactacatcaccgtggaaatgcctagagatgataacgaggaggaagagcgtaaaagaattaaggaactgcaaacgaagaattctcagagaaagaacgatagccagcaatacttcctgcagaaatccggctggagccaggagaagttccaggccacaatccacaagaatagaagattcctggccaagctgctgtactattttgagcaggacggcgtgtgtgcctacaccgggaaccccatcagccctgagcttctggtgtccgacagcaccgagatcgaccacatcatccctatcagcatcagcctggatgacagcatcaacaacaaagtgctggtgctgtctcacgccaaccaggtgaagggccaacagaccccttacgatgccagaatggccggcgccttcaacaagatcaatggaaagttcagcaactgggacgagtatcaaaagtgggtggaaagcagacctttcagccggaaaaaggtgaacaacctcctggagaccagaaacattttcgacagcgagcaggtgcaaaagtttctgagcagaaacctgaatgatacaagatacgccagcagactggtcctgaataccctgcagagctttttcgagaaccaggataccattgtgcgggtggtcaacggcagcttcacccataccctgagaaagaagtggggcgccgatctggacaaaacccgggagacacaccaccaccacgccgtggacgccacactgtgcgcggtgacacccttcgtgaaggtctctagataccactacgccgtgaacgaggagaccggcgagaaattcatgagagaaatcgatgtggagaccggcgaaatcctggacgaaattccatacagagagtacaagaaggccaagcactacgagagaaaaacctaccaggtgaagtggtccaacttccgcgagcagcttaagcccatcacaatccatcctaagatcaagttcagccaccaggttgacagaaaagccaacagaaagctgagcgacgctaccatctactccgttagagagaagacagaggtgaaaacactgaagagcggcaaggagaagatcacgaccgacgaatacaccatcggcaagatcaaggacatctataccgtagacggctgggaagcctttaaaaagaagcaggacaaactgctcatgaaggaattcgatgaaaagacctacgagctgctggtgaccatcgccgccaccacccctgattttcaggaggtggaagaaaagaacggcaaagtgaagagagtgaagcggagccctttcgccgtgtactgtgaggaaaacggcatacccgccatccggaaatacgccaagaagaacaacggccccgtgatccggagcctgaagtactacgacggcaagctgaataagcacatcaacatcaccaaggatgagaagggcaggcccgtggagcagacaaagaacggccggaaagtgactctgcagagcctgaaaccctaccggtatgatatctaccaggacctcgaaaccaaggcctattatacagtgcaactgtactacagcgacctgcgctttgtggagggcgagtacggaatcacagaaaaagagtacatgaagaaggtggccgaacagaccaaaggacaggtggtgcggttctgcttcagcctgcagaagaacgacggcctcgaaatcgagtggaaggattctcaacggtacgacgtgagattctacaatttccaatctgctaacagcatcaacttcaagggcttagaacaggagatgatccctgctgaaaaccagttcaagcagaagccttacaacaatggcgccatcaacctgaacatcgccaagtatggcaaggagggcaagaaactgagaaagttcaacaccgatatcctgggtaaaaagcaccacctgtcttatgagaaagagccgaagaacatcatcaag

SEQ ID NO: 89 - DNA, 318 bp, synthetic construct
  source 1..318: mol_type = other DNA
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 90 - DNA, 225 bp, synthetic construct
  source 1..225: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatc

SEQ ID NO: 91 - DNA, 280 bp, synthetic construct
  source 1..280: mol_type = other DNA
gaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccacc

SEQ ID NO: 92 - DNA, 195 bp, synthetic construct
  source 1..195: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgaca

SEQ ID NO: 93 - DNA, 448 bp, synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 94 - DNA, 179 bp, synthetic construct
  source 1..179: mol_type = other DNA
taagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtt

SEQ ID NO: 95 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 96 - DNA, 300 bp, synthetic construct
  source 1..300: mol_type = other DNA
agctccacggcaagagaattcaaagccgcgggcctgggttccacgcggggccccttacccaaggtgccccgggcgctcatttgcatgtcccacccaacaggtaaacctgacaggtcagtcgcggccgggtacggcctggcggtcagagcaccaaacgtacgagccttgtgatgcggttccattgcatgaaattctcctaaaggccccaagatgaacgggaaagcgcgcggttcgctcaccgtaactaaaacaggtgagagactcccgtgccttataaggcctgtggacggaggcagttgc

SEQ ID NO: 97 - DNA, 300 bp, synthetic construct
  source 1..300: mol_type = other DNA
tgttctccgaacagtacttgtaatatacaggttccccgatccaccaccgtctgcgggtggcggtacagcctctcccagtgtgctttgcgctcatttgcatagagcacggcaccgaacgcagccactgtcccccgcgtccgctgtcccccgtgcaggccaaacccgggcgcggccccgccgacggtgggtacagagagtggtcggctcggcaccggctgctgcgcggcaccggcactcaccctgtccttatggagccctaactccatggctataaatatctaaggggaggaagggtagatc

SEQ ID NO: 98 - DNA, 300 bp, synthetic construct
  source 1..300: mol_type = other DNA
agcgctccgcggagacttctgggacgcggcggctccgactccgccccgcttccggcttatttgcatacagcaattcctagcaggccctgtgctgaatttagtcggaaaactaccatgttcagtcgaaaaagcaaatacttttgtcagatatggccaaaaacttcacttgacttagccgtgtttcatgtaaagcattaaaaggatggagtgattgttcaaatttcataagaagaattcaccttcagtttaaggtggttcgctttctgcacttcaaataccgcggtggacagaccctgtttc

SEQ ID NO: 99 - DNA, 300 bp, synthetic construct
  source 1..300: mol_type = other DNA
gcctgagtcgcgccgccgcctcccaaagacttctgggaaggcggtgcggctcaggctccgccccgcttccggggatatttgcatacgagcattcccagtaattcccagcagccactgtagctatatttggtagaataacgagcactttctgaactccagttaataactgcgttagttgcgttacacattggactaaaacaaatagaagttgaatctctagagcagtggagataagtcgccgtatgtgtacagaaattgcttccgggggctataaatagctggtagtggggctagaacgtc

SEQ ID NO: 100 - DNA, 300 bp, synthetic construct
  source 1..300: mol_type = other DNA
cgcccgcgcagcgcagccgcttcccagagacttctgggacggcagcggctgcggctccgcccctcttccagtataatttgcatgcgaccatggattcccagcagccacctgagtcatatttggtggaacaaaaaccactttctcaatttcagtgaatgaccccatttggttaaggtattgttgcacaaatatcataaaagaagggacgaatggctgaaccggtttttaatggagttcgcccttagcgtaaaagagtttattctatgccctctaaatagttctgggatcaaccgtactact

SEQ ID NO: 101 - DNA, 111 bp, synthetic construct
  source 1..111: mol_type = other DNA
ccccttacccaaggtgccccgggcgctcatttgcatgtcccaccgctcaccgtaactaaaacaggtgagagactcccgtgccttataaggcctgtggacggaggcagttgc

SEQ ID NO: 102 - DNA, 111 bp, synthetic construct
  source 1..111: mol_type = other DNA
acagcctctcccagtgtgctttgcgctcatttgcatagagcacgactcaccctgtccttatggagccctaactccatggctataaatatctaaggggaggaagggtagatc

SEQ ID NO: 103 - DNA, 111 bp, synthetic construct
  source 1..111: mol_type = other DNA
ggctccgactccgccccgcttccggcttatttgcatacagcaatattcaccttcagtttaaggtggttcgctttctgcacttcaaataccgcggtggacagaccctgtttc

SEQ ID NO: 104 - DNA, 111 bp, synthetic construct
  source 1..111: mol_type = other DNA
gctcaggctccgccccgcttccggggatatttgcatacgagcatagtcgccgtatgtgtacagaaattgcttccgggggctataaatagctggtagtggggctagaacgtc

SEQ ID NO: 105 - DNA, 111 bp, synthetic construct
  source 1..111: mol_type = other DNA
gctgcggctccgcccctcttccagtataatttgcatgcgaccatgttcgcccttagcgtaaaagagtttattctatgccctctaaatagttctgggatcaaccgtactact

SEQ ID NO: 106 - RNA, 12 nt, synthetic construct
  source 1..12: mol_type = other RNA
gttattgtactc

SEQ ID NO: 107 - RNA, 60 nt, synthetic construct
  source 1..60: mol_type = other RNA
gaatctacaataataaggcatcttgccgaatttaccgccctacatatgtagggcggtttt

SEQ ID NO: 108 - DNA, 20 bp, synthetic construct
  source 1..20: mol_type = other DNA
ggctaccatcctgaaaactc

SEQ ID NO: 109 - DNA, 20 bp, synthetic construct
  source 1..20: mol_type = other DNA
ggtccatgataataagggcg

SEQ ID NO: 110 - DNA, 22 bp, synthetic construct
  source 1..22: mol_type = other DNA
tgcgttatttgtgcaaccaggt

SEQ ID NO: 111 - DNA, 20 bp, synthetic construct
  source 1..20: mol_type = other DNA
ggctaccatcctgaaaactc

SEQ ID NO: 112 - DNA, 20 bp, synthetic construct
  source 1..20: mol_type = other DNA
ggtccatgataataagggcg

SEQ ID NO: 113 - DNA, 22 bp, synthetic construct
  source 1..22: mol_type = other DNA
tgcgttatttgtgcaaccaggt

SEQ ID NO: 114 - DNA, 20 bp, synthetic construct
  source 1..20: mol_type = other DNA
gccgagatctacaatcctac

SEQ ID NO: 115 - DNA, 20 bp, synthetic construct
  source 1..20: mol_type = other DNA
gtgatgtagtggatctggtc

SEQ ID NO: 116 - DNA, 20 bp, synthetic construct
  source 1..20: mol_type = other DNA
ccttcaaggtgatggacgct

SEQ ID NO: 117 - DNA, 20 bp, synthetic construct
  source 1..20: mol_type = other DNA
gccgagatctacaatcctac

SEQ ID NO: 118 - DNA, 20 bp, synthetic construct
  source 1..20: mol_type = other DNA
gtgatgtagtggatctggtc

SEQ ID NO: 119 - DNA, 20 bp, synthetic construct
  source 1..20: mol_type = other DNA
ccttcaaggtgatggacgct

SEQ ID NO: 120 - RNA, 64 nt, synthetic construct
  source 1..64: mol_type = other RNA
ttacagaatctactaaaacaagactatatgtcgtgtttatcccatcaatttattgatgggattt

SEQ ID NO: 121 - DNA, 7004 bp, synthetic construct
  source 1..7004: mol_type = other DNA
gtgcacgatatcgggtccccaattgctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgtcgtgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccggcctccaggatgaagtgcacagttattgtactcaaaggaatctacaataataaggcatcttgccgaatttaccgccctacatatgtagggcggtttttttttgtgatatcctcgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctctttgtcgagccaccatggcccctaagaagaaaagaaaggtgatgtactctatcggcctcgatctgggcatctctagcgtcggctggtcggtgatcgacgaagagacaggcaaaatcgttgacctgggcgtgcggctgttttctgctaagaacagcgagaagaatctggagagaagaaccagcagaggcgcaagaagactgatcagaagaaagacgaaccgactaaaagacgccaagaaattactggaggccatcggcttctacgaagataaggcccttaaaaatgtctgcccttaccaactgagagtgaagggcctgaccgagggcctgacaaagggcgaactgtacaaggtcgtgctgcacatcgtgaagaaacggggcatcagctacctggacgaggacgacgccgaagctgccaaagaatctcaggactacaaggagcaggtacgcaaaaatgcccaactgctgacaaagtacacccctggacagatccaactgcagagactgaaggagaacaacagagtgaagacaggaatcaacggccagggccactaccaactgaacgtgttcaaggtgagcgcttacgctgacgagctggccaccatcctgaagacccagcaagccctgtatcctaacgaactgaccgacgactggatcgccctgttcgtgcaacctggaattgccgagaatgccggccttatctacagaaagaggccttactaccacggccctggcaacgaggccaacaacagcccttacggaagatggtccgacttccagaaaacaggccagccagccgctaacatcttcgacaagctaatcggaaaggatttccagggcgagctgcgggccagcggcctgagcctgtctgcccagcaatacaacctgctgaacgacctgaccaatctgaaaattgacggagaggtgtccctgtcccccgagcagaaggaattcatcctgaccgagctgatgaccaaggaattcgccagattcggcgtcaacgacattgccaagctgctgggcgtgaaaaaagaacagttgtctggctggcggctggacaagaagggcaagcccgaaatccacaccctgaagggctacagaaactggagaaagatctttgccgaggccggaatcgacctggccacactgccaacagagaccattgactgcctggccaaggttctgaccctgaacaccgagcgggaaggagtggaaaacaccctggcctttgagctgcctgaactggctgagcctgtgaagagcctggtgttggaccattacaaggaactgagccaaagcatctcaacacaggcttggcacagattcagcctgaagacactgcacctgctgatccccgagctgatcaagagcaccagcgagcagaacaccctgctggaacagttccaactgaaggccggcgtgcgcaagcggtactccgactacaagaagctgcctaccaaggaggtgctggccgagatctacaatcctactgtgaacaaaaccgtcagccaggccttcaaggtgatggacgctctgctggagaagtacggcaaggaccagatccactacatcaccgtggaaatgcctagagatgataacgaggaggaagagcgtaaaagaattaaggaactgcaaacgaagaattctcagagaaagaacgatagccagcaatacttcctgcagaaatccggctggagccaggagaagttccaggccacaatccacaagaatagaagattcctggccaagctgctgtactattttgagcaggacggcgtgtgtgcctacaccgggaaccccatcagccctgagcttctggtgtccgacagcaccgagatcgaccacatcatccctatcagcatcagcctggatgacagcatcaacaacaaagtgctggtgctgtctcacgccaaccaggtgaagggccaacagaccccttacgatgccagaatggccggcgccttcaacaagatcaatggaaagttcagcaactgggacgagtatcaaaagtgggtggaaagcagacctttcagccggaaaaaggtgaacaacctcctggagaccagaaacattttcgacagcgagcaggtgcaaaagtttctgagcagaaacctgaatgatacaagatacgccagcagactggtcctgaataccctgcagagctttttcgagaaccaggataccattgtgcgggtggtcaacggcagcttcacccataccctgagaaagaagtggggcgccgatctggacaaaacccgggagacacaccaccaccacgccgtggacgccacactgtgcgcggtgacacccttcgtgaaggtctctagataccactacgccgtgaacgaggagaccggcgagaaattcatgagagaaatcgatgtggagaccggcgaaatcctggacgaaattccatacagagagtacaagaaggccaagcactacgagagaaaaacctaccaggtgaagtggtccaacttccgcgagcagcttaagcccatcacaatccatcctaagatcaagttcagccaccaggttgacagaaaagccaacagaaagctgagcgacgctaccatctactccgttagagagaagacagaggtgaaaacactgaagagcggcaaggagaagatcacgaccgacgaatacaccatcggcaagatcaaggacatctataccgtagacggctgggaagcctttaaaaagaagcaggacaaactgctcatgaaggaattcgatgaaaagacctacgagctgctggtgaccatcgccgccaccacccctgattttcaggaggtggaagaaaagaacggcaaagtgaagagagtgaagcggagccctttcgccgtgtactgtgaggaaaacggcatacccgccatccggaaatacgccaagaagaacaacggccccgtgatccggagcctgaagtactacgacggcaagctgaataagcacatcaacatcaccaaggatgagaagggcaggcccgtggagcagacaaagaacggccggaaagtgactctgcagagcctgaaaccctaccggtatgatatctaccaggacctcgaaaccaaggcctattatacagtgcaactgtactacagcgacctgcgctttgtggagggcgagtacggaatcacagaaaaagagtacatgaagaaggtggccgaacagaccaaaggacaggtggtgcggttctgcttcagcctgcagaagaacgacggcctcgaaatcgagtggaaggattctcaacggtacgacgtgagattctacaatttccaatctgctaacagcatcaacttcaagggcttagaacaggagatgatccctgctgaaaaccagttcaagcagaagccttacaacaatggcgccatcaacctgaacatcgccaagtatggcaaggagggcaagaaactgagaaagttcaacaccgatatcctgggtaaaaagcaccacctgtcttatgagaaagagccgaagaacatcatcaagagcggcggaagcaaaagacctgccgctacaaagaaggccggccaggccaagaaaaagaagtaatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaaggaacccctagtgatggagttggccactccctctctgcgcgctcgctcgctcactgaggccgggcgaccaaaggtcgcccgacgcccgggctttgcccgggcggcctcagtgagcgagcgagcgcgcagaggcatttaattaagcaagctgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtcc

SEQ ID NO: 122 - DNA, 6935 bp, synthetic construct
  source 1..6935: mol_type = other DNA
gtgcacgatatcgggtccccaattgctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgtcggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccggcctccaggatgaagtgcacagttattgtactcaaaggaatctacaataataaggcatcttgccgaatttaccgccctacatatgtagggcggtttttttttgtgatatcctcgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctctttgtcgagccaccatggcccctaagaagaaaagaaaggtgatgtactctatcggcctcgatctgggcatctctagcgtcggctggtcggtgatcgacgaagagacaggcaaaatcgttgacctgggcgtgcggctgttttctgctaagaacagcgagaagaatctggagagaagaaccagcagaggcgcaagaagactgatcagaagaaagacgaaccgactaaaagacgccaagaaattactggaggccatcggcttctacgaagataaggcccttaaaaatgtctgcccttaccaactgagagtgaagggcctgaccgagggcctgacaaagggcgaactgtacaaggtcgtgctgcacatcgtgaagaaacggggcatcagctacctggacgaggacgacgccgaagctgccaaagaatctcaggactacaaggagcaggtacgcaaaaatgcccaactgctgacaaagtacacccctggacagatccaactgcagagactgaaggagaacaacagagtgaagacaggaatcaacggccagggccactaccaactgaacgtgttcaaggtgagcgcttacgctgacgagctggccaccatcctgaagacccagcaagccctgtatcctaacgaactgaccgacgactggatcgccctgttcgtgcaacctggaattgccgagaatgccggccttatctacagaaagaggccttactaccacggccctggcaacgaggccaacaacagcccttacggaagatggtccgacttccagaaaacaggccagccagccgctaacatcttcgacaagctaatcggaaaggatttccagggcgagctgcgggccagcggcctgagcctgtctgcccagcaatacaacctgctgaacgacctgaccaatctgaaaattgacggagaggtgtccctgtcccccgagcagaaggaattcatcctgaccgagctgatgaccaaggaattcgccagattcggcgtcaacgacattgccaagctgctgggcgtgaaaaaagaacagttgtctggctggcggctggacaagaagggcaagcccgaaatccacaccctgaagggctacagaaactggagaaagatctttgccgaggccggaatcgacctggccacactgccaacagagaccattgactgcctggccaaggttctgaccctgaacaccgagcgggaaggagtggaaaacaccctggcctttgagctgcctgaactggctgagcctgtgaagagcctggtgttggaccattacaaggaactgagccaaagcatctcaacacaggcttggcacagattcagcctgaagacactgcacctgctgatccccgagctgatcaagagcaccagcgagcagaacaccctgctggaacagttccaactgaaggccggcgtgcgcaagcggtactccgactacaagaagctgcctaccaaggaggtgctggccgagatctacaatcctactgtgaacaaaaccgtcagccaggccttcaaggtgatggacgctctgctggagaagtacggcaaggaccagatccactacatcaccgtggaaatgcctagagatgataacgaggaggaagagcgtaaaagaattaaggaactgcaaacgaagaattctcagagaaagaacgatagccagcaatacttcctgcagaaatccggctggagccaggagaagttccaggccacaatccacaagaatagaagattcctggccaagctgctgtactattttgagcaggacggcgtgtgtgcctacaccgggaaccccatcagccctgagcttctggtgtccgacagcaccgagatcgaccacatcatccctatcagcatcagcctggatgacagcatcaacaacaaagtgctggtgctgtctcacgccaaccaggtgaagggccaacagaccccttacgatgccagaatggccggcgccttcaacaagatcaatggaaagttcagcaactgggacgagtatcaaaagtgggtggaaagcagacctttcagccggaaaaaggtgaacaacctcctggagaccagaaacattttcgacagcgagcaggtgcaaaagtttctgagcagaaacctgaatgatacaagatacgccagcagactggtcctgaataccctgcagagctttttcgagaaccaggataccattgtgcgggtggtcaacggcagcttcacccataccctgagaaagaagtggggcgccgatctggacaaaacccgggagacacaccaccaccacgccgtggacgccacactgtgcgcggtgacacccttcgtgaaggtctctagataccactacgccgtgaacgaggagaccggcgagaaattcatgagagaaatcgatgtggagaccggcgaaatcctggacgaaattccatacagagagtacaagaaggccaagcactacgagagaaaaacctaccaggtgaagtggtccaacttccgcgagcagcttaagcccatcacaatccatcctaagatcaagttcagccaccaggttgacagaaaagccaacagaaagctgagcgacgctaccatctactccgttagagagaagacagaggtgaaaacactgaagagcggcaaggagaagatcacgaccgacgaatacaccatcggcaagatcaaggacatctataccgtagacggctgggaagcctttaaaaagaagcaggacaaactgctcatgaaggaattcgatgaaaagacctacgagctgctggtgaccatcgccgccaccacccctgattttcaggaggtggaagaaaagaacggcaaagtgaagagagtgaagcggagccctttcgccgtgtactgtgaggaaaacggcatacccgccatccggaaatacgccaagaagaacaacggccccgtgatccggagcctgaagtactacgacggcaagctgaataagcacatcaacatcaccaaggatgagaagggcaggcccgtggagcagacaaagaacggccggaaagtgactctgcagagcctgaaaccctaccggtatgatatctaccaggacctcgaaaccaaggcctattatacagtgcaactgtactacagcgacctgcgctttgtggagggcgagtacggaatcacagaaaaagagtacatgaagaaggtggccgaacagaccaaaggacaggtggtgcggttctgcttcagcctgcagaagaacgacggcctcgaaatcgagtggaaggattctcaacggtacgacgtgagattctacaatttccaatctgctaacagcatcaacttcaagggcttagaacaggagatgatccctgctgaaaaccagttcaagcagaagccttacaacaatggcgccatcaacctgaacatcgccaagtatggcaaggagggcaagaaactgagaaagttcaacaccgatatcctgggtaaaaagcaccacctgtcttatgagaaagagccgaagaacatcatcaagagcggcggaagcaaaagacctgccgctacaaagaaggccggccaggccaagaaaaagaagtaatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaaggaacccctagtgatggagttggccactccctctctgcgcgctcgctcgctcactgaggccgggcgaccaaaggtcgcccgacgcccgggctttgcccgggcggcctcagtgagcgagcgagcgcgcagaggcatttaattaagcaagctgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtcc

SEQ ID NO: 123 - DNA, 6956 bp, synthetic construct
  source 1..6956: mol_type = other DNA
gtgcacgatatcgggtccccaattgctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgtcggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccggcctccaggatgaagtgcacagttattgtactcaaaggaatctacaataataaggcatcttgccgaatttaccgccctacatatgtagggcggtttttttttgtgatatcctcgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctctttgtcgagccaccatggcccctgctgccaagcgcgtgaagctggacggcagctctggcggaagttccggaatgtactctatcggcctcgatctgggcatctctagcgtcggctggtcggtgatcgacgaagagacaggcaaaatcgttgacctgggcgtgcggctgttttctgctaagaacagcgagaagaatctggagagaagaaccagcagaggcgcaagaagactgatcagaagaaagacgaaccgactaaaagacgccaagaaattactggaggccatcggcttctacgaagataaggcccttaaaaatgtctgcccttaccaactgagagtgaagggcctgaccgagggcctgacaaagggcgaactgtacaaggtcgtgctgcacatcgtgaagaaacggggcatcagctacctggacgaggacgacgccgaagctgccaaagaatctcaggactacaaggagcaggtacgcaaaaatgcccaactgctgacaaagtacacccctggacagatccaactgcagagactgaaggagaacaacagagtgaagacaggaatcaacggccagggccactaccaactgaacgtgttcaaggtgagcgcttacgctgacgagctggccaccatcctgaagacccagcaagccctgtatcctaacgaactgaccgacgactggatcgccctgttcgtgcaacctggaattgccgagaatgccggccttatctacagaaagaggccttactaccacggccctggcaacgaggccaacaacagcccttacggaagatggtccgacttccagaaaacaggccagccagccgctaacatcttcgacaagctaatcggaaaggatttccagggcgagctgcgggccagcggcctgagcctgtctgcccagcaatacaacctgctgaacgacctgaccaatctgaaaattgacggagaggtgtccctgtcccccgagcagaaggaattcatcctgaccgagctgatgaccaaggaattcgccagattcggcgtcaacgacattgccaagctgctgggcgtgaaaaaagaacagttgtctggctggcggctggacaagaagggcaagcccgaaatccacaccctgaagggctacagaaactggagaaagatctttgccgaggccggaatcgacctggccacactgccaacagagaccattgactgcctggccaaggttctgaccctgaacaccgagcgggaaggagtggaaaacaccctggcctttgagctgcctgaactggctgagcctgtgaagagcctggtgttggaccattacaaggaactgagccaaagcatctcaacacaggcttggcacagattcagcctgaagacactgcacctgctgatccccgagctgatcaagagcaccagcgagcagaacaccctgctggaacagttccaactgaaggccggcgtgcgcaagcggtactccgactacaagaagctgcctaccaaggaggtgctggccgagatctacaatcctactgtgaacaaaaccgtcagccaggccttcaaggtgatggacgctctgctggagaagtacggcaaggaccagatccactacatcaccgtggaaatgcctagagatgataacgaggaggaagagcgtaaaagaattaaggaactgcaaacgaagaattctcagagaaagaacgatagccagcaatacttcctgcagaaatccggctggagccaggagaagttccaggccacaatccacaagaatagaagattcctggccaagctgctgtactattttgagcaggacggcgtgtgtgcctacaccgggaaccccatcagccctgagcttctggtgtccgacagcaccgagatcgaccacatcatccctatcagcatcagcctggatgacagcatcaacaacaaagtgctggtgctgtctcacgccaaccaggtgaagggccaacagaccccttacgatgccagaatggccggcgccttcaacaagatcaatggaaagttcagcaactgggacgagtatcaaaagtgggtggaaagcagacctttcagccggaaaaaggtgaacaacctcctggagaccagaaacattttcgacagcgagcaggtgcaaaagtttctgagcagaaacctgaatgatacaagatacgccagcagactggtcctgaataccctgcagagctttttcgagaaccaggataccattgtgcgggtggtcaacggcagcttcacccataccctgagaaagaagtggggcgccgatctggacaaaacccgggagacacaccaccaccacgccgtggacgccacactgtgcgcggtgacacccttcgtgaaggtctctagataccactacgccgtgaacgaggagaccggcgagaaattcatgagagaaatcgatgtggagaccggcgaaatcctggacgaaattccatacagagagtacaagaaggccaagcactacgagagaaaaacctaccaggtgaagtggtccaacttccgcgagcagcttaagcccatcacaatccatcctaagatcaagttcagccaccaggttgacagaaaagccaacagaaagctgagcgacgctaccatctactccgttagagagaagacagaggtgaaaacactgaagagcggcaaggagaagatcacgaccgacgaatacaccatcggcaagatcaaggacatctataccgtagacggctgggaagcctttaaaaagaagcaggacaaactgctcatgaaggaattcgatgaaaagacctacgagctgctggtgaccatcgccgccaccacccctgattttcaggaggtggaagaaaagaacggcaaagtgaagagagtgaagcggagccctttcgccgtgtactgtgaggaaaacggcatacccgccatccggaaatacgccaagaagaacaacggccccgtgatccggagcctgaagtactacgacggcaagctgaataagcacatcaacatcaccaaggatgagaagggcaggcccgtggagcagacaaagaacggccggaaagtgactctgcagagcctgaaaccctaccggtatgatatctaccaggacctcgaaaccaaggcctattatacagtgcaactgtactacagcgacctgcgctttgtggagggcgagtacggaatcacagaaaaagagtacatgaagaaggtggccgaacagaccaaaggacaggtggtgcggttctgcttcagcctgcagaagaacgacggcctcgaaatcgagtggaaggattctcaacggtacgacgtgagattctacaatttccaatctgctaacagcatcaacttcaagggcttagaacaggagatgatccctgctgaaaaccagttcaagcagaagccttacaacaatggcgccatcaacctgaacatcgccaagtatggcaaggagggcaagaaactgagaaagttcaacaccgatatcctgggtaaaaagcaccacctgtcttatgagaaagagccgaagaacatcatcaagggcagctctggcggaagttccggacctgctgccaagcgcgtgaagctggactaatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaaggaacccctagtgatggagttggccactccctctctgcgcgctcgctcgctcactgaggccgggcgaccaaaggtcgcccgacgcccgggctttgcccgggcggcctcagtgagcgagcgagcgcgcagaggcatttaattaagcaagctgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcacgatatcgggtcc

SEQ ID NO: 124 - DNA, 27 bp, synthetic construct
  source 1..27: mol_type = other DNA
cctgctgccaagcgcgtgaagctggac

SEQ ID NO: 125 - protein, 9 aa, synthetic construct
PAAKRVKLD

SEQ ID NO: 126 - DNA, 24 bp, synthetic construct
  source 1..24: mol_type = other DNA
ggcagctctggcggaagttccgga

SEQ ID NO: 127 - protein, 8 aa, synthetic construct
GSSGGSSG

SEQ ID NO: 128 - DNA, 249 bp, synthetic construct
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 129 - RNA, 101 nt, synthetic construct
  source 1..101: mol_type = other RNA
gagtttgtgacccacgcctgctcccgttattgtactcaaaggaatctacaataataaggcatcttgccgaatttaccgccctacatatgtagggcggtttt

SEQ ID NO: 130 - DNA, 25 bp, Homo sapiens
  source 1..25: mol_type = genomic DNA
gagtttgtgacccacgcctgctccc

SEQ ID NO: 131 - RNA, 101 nt, synthetic construct
  source 1..101: mol_type = other RNA
aggaggcagcagagaactcccagaagttattgtactcaaaggaatctacaataataaggcatcttgccgaatttaccgccctacatatgtagggcggtttt

SEQ ID NO: 132 - DNA, 25 bp, synthetic construct
  source 1..25: mol_type = other DNA
aggaggcagcagagaactcccagaa

SEQ ID NO: 133 - protein, 133 aa, Bacillus sp.
GDLVRVDLFEKDDKYYMVPIYVPDTVCSELPKKVVASSKGYEQWLTLDNSFTFKFSLYPYDLVRLVKGDEDRFLYFGTLDIDSDRLNFKDVNKPSKKNEYRYSLKTIEDLEKYEVGVLGDLRLVRKETRRNFH

SEQ ID NO: 134 - protein, 152 aa, Enterococcus sp.
PYRYDIYQDLETKAYYTVQLYYSDLRFVEGEYGITEKEYMKKVAEQTKGQVVRFCFSLQKNDGLEIEWKDSQRYDVRFYNFQSANSINFKGLEQEMIPAENQFKQKPYNNGAINLNIAKYGKEGKKLRKFNTDILGKKHHLSYEKEPKNIIK

SEQ ID NO: 135 - protein, 143 aa, Staphylococcus sp.
PYRFDVYLTDKGYKFITISYLDVLKKDNYYYILKEKYEELKIKKSISDTDQFIGSFYYNDLIKINDQIFKVVGVNNDLLNRIELDLLDISYKEYCKINNIKTNRIIKGITKKITNIEKFSTDVLGNLYKAHSNHPQLIFKQRD

SEQ ID NO: 136 - protein, 154 aa, unidentified
PWRTDVYYNHEKEQYEIMGIKYADLKFKGDNYGITKARYQEIKEEEGVSEESEFLFSLYRGDRIQVSNGEDKIDLLFLSRSNPAKKGYVELKPIDRNQLNGKEVVSVYGAASGGRLKKQFVKKNHTLHKVNTDILGNPFYIKKESDQPKNILDL

SEQ ID NO: 137 - DNA, 33 bp, synthetic construct
  source 1..33: mol_type = other DNA
tcgtcggcagcgtcagatgtgtataagagacag

SEQ ID NO: 138 - DNA, 34 bp, synthetic construct
  source 1..34: mol_type = other DNA
gtctcgtgggctcggagatgtgtataagagacag

SEQ ID NO: 139 - protein, 9 aa, synthetic construct
LAGLIDADG

SEQ ID NO: 140 - RNA, 90 nt, synthetic construct
  source 1..90: mol_type = other RNA
gttattgtactctcaataaaaagttattgagaatctacaataataaggcatcttgccgaatttaccgccctacatatgtagggcggtttt

SEQ ID NO: 141 - RNA, 76 nt, synthetic construct
  source 1..76: mol_type = other RNA
gttattgtactcaaaggaatctacaataataaggcatcttgccgaatttaccgccctacatatgtagggcggtttt

SEQ ID NO: 142 - RNA, 110 nt, synthetic construct
  source 1..110: mol_type = other RNA
gtcatagttccattaaagccaaaagtggctttgatgtttctatgataagggtttcgacccgtggcgtcggggatcgcctgcccattgaaatgggcttctccccatttatt

SEQ ID NO: 143 - protein, 54 aa, Bacillus sp.
MRELDYRIGLDIGTNSIGWGVIELSWNKDRERYEKVRIVDQGVRMFDRAEMPKT

SEQ ID NO: 144 - protein, 59 aa, Bacillus sp.
DTSGLKKEKRSKFLPPISDEITNPIVKRALTQARKVVNAIIRRHGSPHSVHIELARELS

SEQ ID NO: 145 - protein, 120 aa, Bacillus sp.
ILNPTGYDIVRYKLWKEQGERCAYSLKEIPADTFFNELKKERNGAPILEVDHILPYSQSFIDSYHNKVLVYSDENRKKGNRIPYTYFLETNKDWEAFERYVRSNKFFSKKKREYLLKRAY

SEQ ID NO: 146 - protein, 148 aa, Bacillus sp.
NDTRYASTFLKNFIEQNLQFKEAEDNPRKRRVQTVNGVITAHFRKRWGLEKDRQETYLHHAMDAIIVACTDHHMVTRVTEYYQIKESNKSVKKPYFPMPWEGFRDELLSHLASQPIAKKISEELKAGYQSLDYIFVSRMPKRSITGAA

SEQ ID NO: 147 - protein, 33 aa, Enterococcus sp.
MYSIGLDLGISSVGWSVIDEETGKIVDLGVRLF

SEQ ID NO: 148 - protein, 49 aa, Enterococcus sp.
KLPTKEVLAEIYNPTVNKTVSQAFKVMDALLEKYGKDQIHYITVEMPRD

SEQ ID NO: 149 - protein, 120 aa, Enterococcus sp.
QEKFQATIHKNRRFLAKLLYYFEQDGVCAYTGNPISPELLVSDSTEIDHIIPISISLDDSINNKVLVLSHANQVKGQQTPYDARMAGAFNKINGKFSNWDEYQKWVESRPFSRKKVNNLL

SEQ ID NO: 150 - protein, 150 aa, Enterococcus sp.
SRNLNDTRYASRLVLNTLQSFFENQDTIVRVVNGSFTHTLRKKWGADLDKTRETHHHHAVDATLCAVTPFVKVSRYHYAVNEETGEKFMREIDVETGEILDEIPYREYKKAKHYERKTYQVKWSNFREQLKPITIHPKIKFSHQVDRKAN

SEQ ID NO: 151 - protein, 40 aa, Staphylococcus sp.
MVTKYILGLDIGITSVGYGIINYEDKTIIDAGVRLFPEAN

SEQ ID NO: 152 - protein, 53 aa, Staphylococcus sp.
DLAKTNKIPKNMIDEFILSPVVKRTFGQAINVINKVIEKYGVPEDIIIELARE

SEQ ID NO: 153 - protein, 115 aa, Staphylococcus sp.
QNAKRLVEKIRLHDEQEGKCLYSLESIPLEDLINNPQYYEVDHIIPRSVSFDNSYQNKVLVKQTENSKKSNRTPYQYFNSGETKLSYNQFKQHVLNLSKSKDRISKKKKEYLLEE

SEQ ID NO: 154 - protein, 124 aa, Staphylococcus sp.
RYATRELTNYLKAYFSANDMDVKVKTINGSFTDYLRKVWKFKKERNHGYKHHAEDALIIANADFLFKENKKLKKANAILEQPSLDNGKSDATVENDNEYVETFSIPKQVNDIKEFRDFKFSHRV

SEQ ID NO: 155 - protein, 42 aa, unidentified
MSKVAKNMTRVNLGFDIGIASVGWSVLDNQTGKILETGVSIF

SEQ ID NO: 156 - protein, 48 aa, unidentified
KVDVKYLTENLYNPVVRKSVKQAMDIFNALFEKYANIDYVVIEMPRDD

SEQ ID NO: 157 - protein, 116 aa, unidentified
DLQLEAQLRKRKKLRQKIRMWYQQRGKCPYSGKTIAAVDLFHQDNQFEIDHIIPLSVSFDDGQNNKVLCYSEMNQEKGKQTPYAFMSRGGGQGFSALQAYVKSNNRLENAKKRNLL

SEQ ID NO: 158 - protein, 138 aa, unidentified
IARNLVDTRYASRIVLNELQQFVRSKELDTRVTVIRGKLTSKLRDRWRLNKSRETHHHHAVDAAVIAVSPMLKMWEKNAEIIPLKVDENTVDLKSGEIITDQEYAAQMYELPYARFLEQMPELHKKIKFHHQVDKKMN